(19) United States
(12) Patent Application Publication
(10) Pub. No.: US 2011/0071141 A1
(43) Pub. Date: Mar. 24, 2011

(54) Pesticidial Condensed-Ring Aryl Compounds

(75) Inventors: Tetsuya Murata, Tochigi (JP); Yasushi Yoneta, Saitama (JP); Jun Mihara, Tochigi (JP); Kei Domon, Tochigi (JP); Mamoru Hatazawa, Ibaraki (JP); Koichi Araki, Ibaraki (JP); Eiichi Shimojo, Tochigi (JP); Katsuhiko Shibuya, Tochigi (JP); Teruyuki Ichihara, Tochigi (JP); Ulrich Görgens, Ratingen (DE); Arnd Voerste, Koln (DE); Angela Becker, Dusseldorf (DE); Eva-Maria Franken, Limonest (FR); Klaus-Helmut Müller, Dusseldorf (DE)

(73) Assignee: Bayer CropScience AG

(21) Appl. No.: 12/922,501

(22) PCT Filed: Mar. 13, 2009
(86) PCT No.: PCT/EP09/01841

§ 371 Date: Dec. 1, 2010

(30) Foreign Application Priority Data
Mar. 14, 2008 (JP) 2008-65647
Apr. 30, 2008 (JP) 2008-118338
Aug. 13, 2008 (JP) 2008-208394

(51) Int. Cl. A01N 43/80; A01N 43/88; A01N 43/78; A01N 43/36; C07D 419/10; C07D 413/14; C07D 417/14; C07D 413/10; C07D 207/20; A01N 43/86; A01P 5/00; C07D 261/04; A61K 31/422; A61K 31/4439; A61K 31/4709; A61K 31/40; A61K 31/506; A61K 31/427; A61K 31/502; A61K 31/536; A61K 31/5415; A61P 33/00; A01P 7/04; A01P 7/02

(52) U.S. Cl. 514/222.8; 514/230.5; 514/248; 514/256; 514/275; 514/314; 514/339; 514/370; 514/378; 514/429; 544/2; 544/63; 544/90; 544/92; 544/237; 544/331; 544/333; 546/167; 546/272.1; 548/181; 548/240; 548/565

(57) ABSTRACT

Condensed-ring aryl compounds of formula (I) and use of the same as a agrochemical for controlling noxious organisms wherein (X)ₘQ, A, R¹, (Y)ₙ and the grouping —W¹—W²—W³—W⁴— are as defined herein.

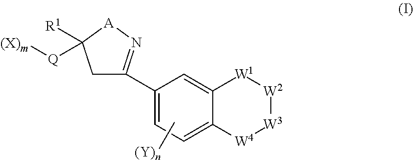

The present invention relates to novel condensed-ring aryl compounds and use of the same as a agrochemical for controlling noxious organisms.

From Japanese Patent Application Laid-open No. 2007-91708 it known that dihydroazole-substituted benzamide compounds may be used as noxious organism control agent. The same is true for the 5-membered heterocyclic compounds as described in WO2007/12 3853 and for the pyrazoline compounds as described in WO2007/12 3855.

It is also known that certain isoxazoline derivatives can be used as noxious organisms control agent (e.g. WO2005/085216, WO2007/026965, WO2007/074789, WO2007/070606, WO2007/075459, WO2007/079162, WO2007/105814, WO2007/12 5984, Japanese Patent Application Laid-open No. 2007-16017, Japanese Patent Application Laid-open No. 2007-106756, and Japanese Patent Application Laid-open No. 2007-308471, WO2007/026965, and WO2007/105814).

WO2005/085216 and its English equivalent EP-A-1 731 512 discloses certain arylisoxazoline compounds having condensed rings and which are supposed to exhibit insecticidal action.

Inventors of the present invention intensively studied to develop a novel pesticidal compound which exhibits a excellent pesticidal effect and has a broad spectrum. As a result, the inventors found novel condensed-ring aryl compounds, which have high activity, a broad spectrum and safety and also are effective against pests that are resistant to an organic phosphorous agent or a carbamate agent.

Thus, this invention is directed to condensed-ring aryl compounds of formula (I)

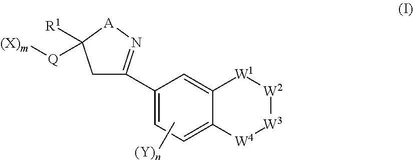

wherein
X represents halogen; nitro; cyano; hydroxy; thiol; amino; C₁₋₁₂ alkyl, C₁₋₁₂ haloalkyl, C₁₋₁₂ alkoxy, C₁₋₁₂ haloalkoxy, C₁₋₁₂ alkylsulfenyl, C₁₋₁₂ alkylsulfinyl, C₁₋₁₂ alkyl-sulfonyl, C₁₋₁₂haloalkylsulfenyl, C₁₋₁₂ haloalkylsulfinyl, C₁₋₁₂haloalkylsulfonyl, C₁₋₁₂ alkylamino, C₂₋₂₄ dialkylamino, C₁₋₁₂ acylamino, C₁₋₁₂ alkoxy-carbonylamino, C₁₋₁₂ haloalkoxy-carbonylamino, C₁₋₁₂ alkylsulfonylamino, or C₁₋₁₂ haloalkylsulfonylamino; preferably chloro, bromo, iodo, fluoro; nitro; cyano; hydroxy; thiol; amino; C₁₋₆ alkyl, C₁₋₆haloalkyl, C₁₋₆ alkoxy, C₁₋₆ haloalkoxy, C₁₋₆ alkylsulfenyl, C₁₋₆alkylsulfinyl, C₁₋₆ alkylsulfonyl, C₁₋₆ haloalkylsulfenyl, C₁₋₆ haloalkylsulfinyl, C₁₋₆haloalkylsulfonyl, C₁₋₆ alkylamino, C₂₋₁₂ (total carbon number) dialkylamino, C₁₋₆ acylamino, C₁₋₆alkoxy-carbonylamino, C₁₋₆haloalkoxy-carbonylamino, C₁₋₆ alkylsulfonylamino, or C₁₋₆ haloalkylsulfonylamino;
Q represents substitutable phenyl, substitutable naphtyl or a substitutable 5- or 6-membered heterocyclic group; preferably represents a optionally substituted group selected among Q-1 to Q-54

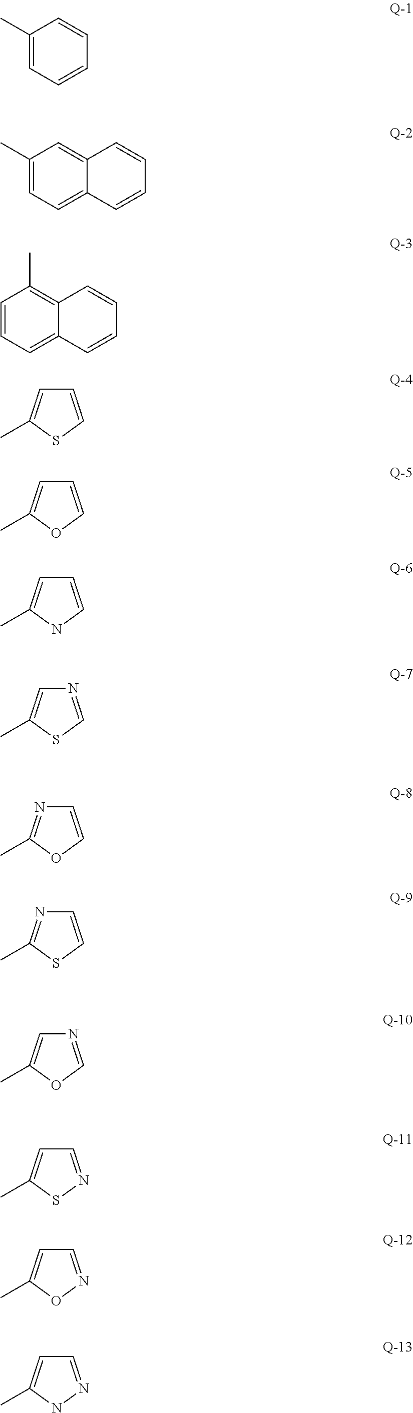

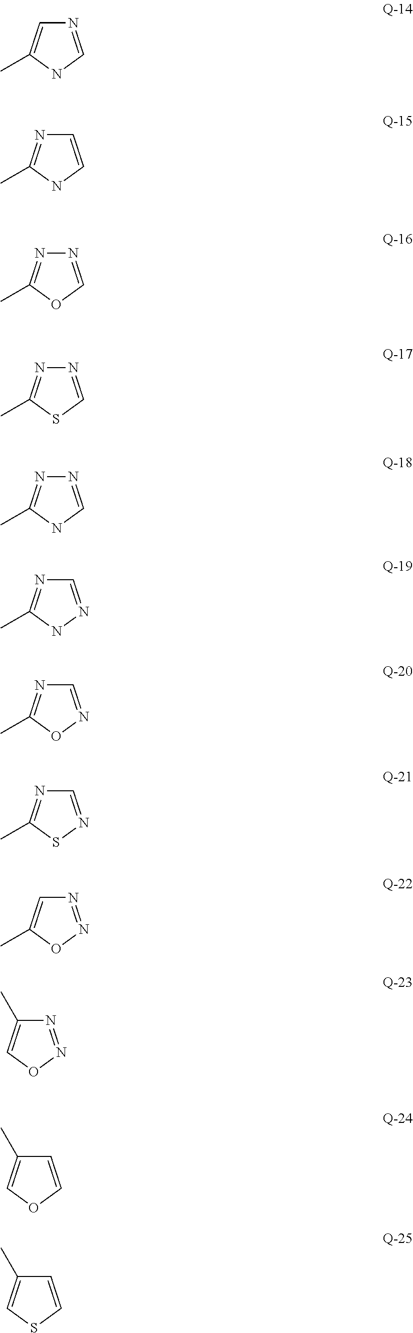

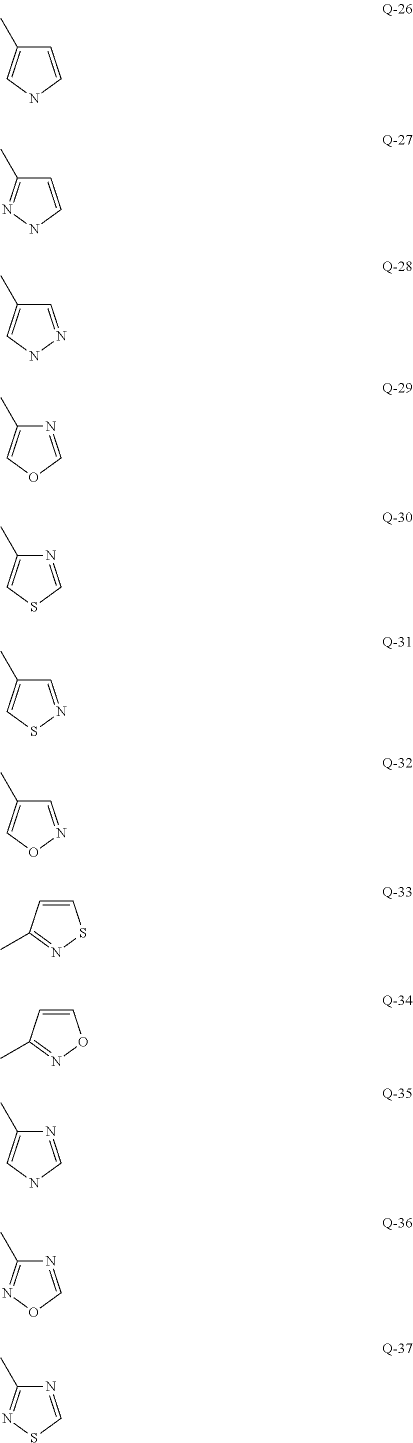

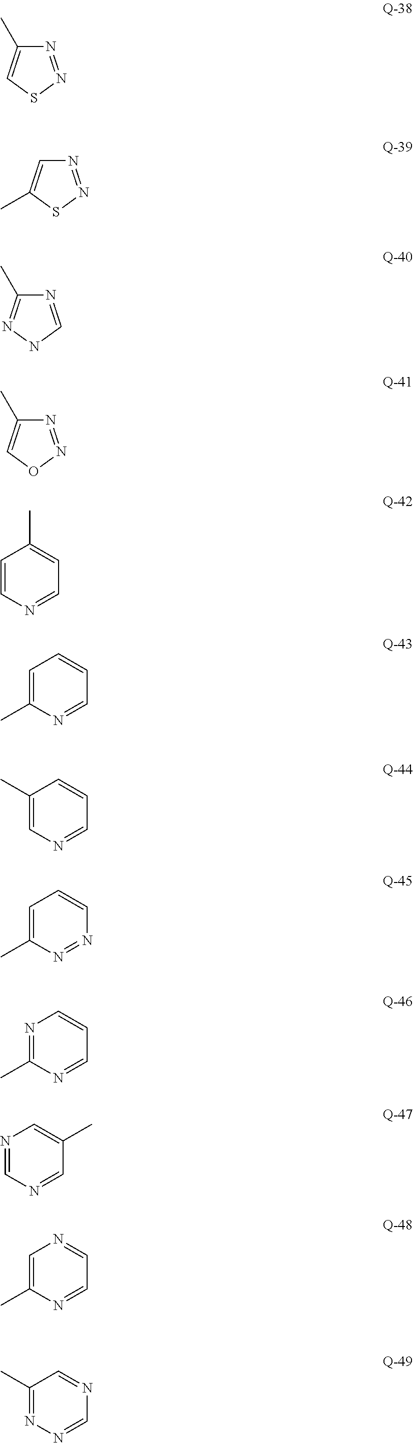

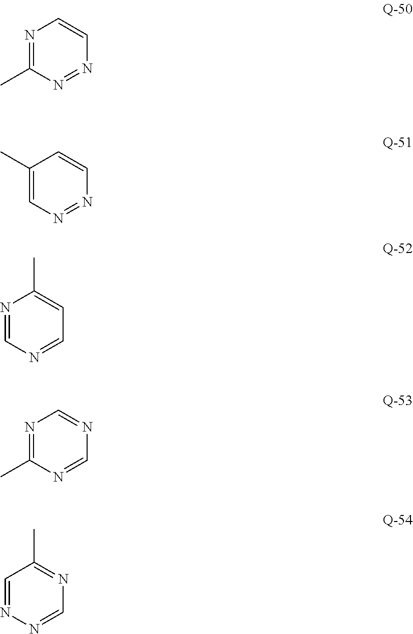

Y represents halogen; nitro; cyano; hydroxy; thiol; amino; C₁₋₁₂ alkyl, C₁₋₁₂ haloalkyl, C₃₋₈ cycloalkyl, C₃₋₈ cyclohaloalkyl, C₁₋₁₂ alkenyl, C₂₋₁₂ haloalkenyl, C₁₋₁₂ alkoxy, C₁₋₁₂ haloalkoxy, C₁₋₁₂ alkylsulfenyl, C₁₋₁₂ alkylsulfinyl, C₁₋₁₂ alkylsulfonyl, C₁₋₁₂ haloalkylsulfenyl, C₁₋₁₂ haloalkylsulfinyl, C₁₋₁₂ haloalkylsulfonyl, C₁₋₁₂ alkylamino, C₂₋₂₄ (total carbon number) dialkyl-amino, C₁₋₁₂ aminocarbonyl, C₁₋₁₂ alkylamino-carbonyl, C₂₋₂₄ (total carbon number) dialkylamino-1-carbonyl, C₁₋₁₂ acylamino, C₁₋₁₂ alkoxy-carbonylamino, benzyloxy-carbonylamino, C₁₋₁₂ (haloalkoxy)-carbonylamino, C₁₋₁₂ alkylsulfonylamino, C₁₋₁₂ haloalkylsulfonylamino, or C₃₋₃₆ (total carbon number) trialkylsilyl; preferably represents chloro, bromo, iodo, fluoro; nitro; cyano; hydroxy; thiol; amino; C₁₋₆ alkyl, C₁₋₆ haloalkyl, C₃₋₆ cycloalkyl, C₃₋₆ cyclohaloalkyl, C₁₋₆ alkenyl, haloalkenyl, C₁₋₆ alkoxy, C₁₋₆ haloalkoxy, C₁₋₆ alkylsulfenyl, C₁₋₆ alkylsulfinyl, C₁₋₆ alkylsulfonyl, C₁₋₆ haloalkylsulfenyl, C₁₋₆ haloalkylsulfinyl, C₁₋₆ haloalkylsulfonyl, C₁₋₆ alkyl-amino, C₂₋₁₂ (total carbon number) dialkylamino, aminocarbonyl, C₁₋₆ alkylamino-carbonyl, C₂₋₁₂ (total carbon number) dialkylamino-carbonyl, C₁₋₆ acylamino, C₁₋₆ alkoxy-carbonylamino, benzyloxy-carbonylamino, C₁₋₆ (haloalkoxy)-carbonylamino, C₁₋₆ alkylsulfonylamino, C₁₋₆ haloalkylsulfonylamino, or C₃₋₁₈ (total carbon number) trialkylsilyl; more preferably represents chloro, bromo, iodo, fluoro; nitro; cyano; hydroxy; thiol; amino; C₁₋₆ alkyl, C₁₋₆ haloalkyl, C₃₋₆ cycloalkyl, C₃₋₆ cyclohaloalkyl, C₁₋₆ alkenyl, haloalkenyl, C₁₋₆ alkoxy, C₁₋₆ haloalkoxy, C₁₋₆ alkylsulfenyl, C₁₋₆ alkylsulfinyl, C₁₋₆ alkylsulfonyl, C₁₋₆ haloalkylsulfenyl, C₁₋₆ haloalkylsulfinyl, C₁₋₆ haloalkylsulfonyl, C₁₋₆ alkylamino, C₂₋₁₂ (total carbon number) dialkylamino; most preferably represents chloro, bromo, iodo, fluoro, cyano, amino, C₁₋₁₂ alkylamino, C₁₋₁₂ haloalkylsulfenyl, C₁₋₁₂ haloalkyl;
R¹ represents cyano; C₁₋₁₂ alkyl, C₃₋₈ cycloalkyl, C₄₋₂₀ (total carbon number) alkyl-cycloalkyl, C₄₋₂₀ (total carbon number) cycloalkylalkyl, C₂₋₁₂ alkenyl, C₂₋₁₂ alkynyl, C₁₋₁₂ haloalkyl, or C₃₋₈ halocycloalkyl; preferably cyano; C₁₋₆ alkyl, C₃₋₆ cycloalkyl, C₄₋₁₂ (total carbon number) alkylcycloalkyl, C₄₋₁₂ (total carbon number) cycloalkylalkyl, C₂₋₆ alkenyl, C₂₋₆ alkynyl, C₁₋₆ haloalkyl, or C₃₋₆ halocycloalkyl; preferably represents C₁₋₆ haloalkyl, most preferably CF₃;
m represents 0, 1, 2, 3, 4 or 5;
n represents 0, 1, 2 or 3;
A represents O, S, CH₂ or N—R²; preferably O;
R² represents hydrogen; cyano; formyl; C₁₋₁₂ alkyl, C₂₋₁₂ alkenyl, C₂₋₁₂ alkynyl, C₃₋₈ cycloalkyl, C₄₋₂₀ (total carbon number) alkylcycloalkyl, C₄₋₂₀ (total carbon number) cycloalkylalkyl, C₁₋₁₂ haloalkyl, C₁₋₁₂ alkylsulfonyl, C₁₋₁₂ haloalkylsulfonyl, phenyl, C₁₋₁₂ alkyl-carbonyl, C₁₋₁₂ alkoxy-carbonyl, C₁₋₁₂ alkylamino-carbonyl, or C₂₋₂₄ dialkylamino-carbonyl; preferably represents hydrogen; cyano; formyl; C₁₋₆ alkyl, C₂₋₆ alkenyl, C₂₋₆ alkynyl, C₃₋₆ cycloalkyl, C₄₋₁₂ (total carbon number) alkylcycloalkyl, C₄₋₁₂ (total curbon number) cycloalkylalkyl, C₁₋₆ haloalkyl, C₁₋₆ alkylsulfonyl, C₁₋₆ haloalkylsulfonyl, phenyl, C₁₋₆ alkyl-carbonyl, C₁₋₆ alkoxy-carbonyl, C₁₋₆ alkylamino-carbonyl, or C₂₋₁₂ (total carbon number) dialkylamino-carbonyl;
W¹, W², W³ and W⁴ each independently may be omitted to yield a 4- or 5-membered ring, and/or each independently represents a single bond, CH₂, CH, N, —N⁺(O⁻)—, —S(O)—, —S(O)₂—, —O—S(O)—, O, S, C(R³)—R³, C—R³, C—R⁴, C(R³)—R⁴, C(R⁴)—R⁴, C—N(R³)—R³, C(R³)—N(R³)—N(R³)—R³, C—N(R³)—N(R³)—R³, C(R³)—N(R⁴)—N(R³)—R³, C—N(R⁴)—N(R³)—R³, C(R³)—N(R³)—OR³, C—N(R³)—OR³, C(R³)—OR³, C—OR³, C(R³)—SR³, C—SR³, C—N₃, N—R³, N—OR³, N—N(R³)—R³, N—R⁴, or C═U under the prerequisite that (i) not more than two of W¹, W², W³ and W⁴ are simultaneously omitted, and/or (ii) not more than two of W¹, W², W³ and W⁴ represent O, S, N—R³ or N—R⁴, C—N(R³)—R³, C—N(R³)—N(R³)—R³, C—N(R⁴)—N(R³)—R³, C(R³)—N(R³)—OR³, C—N(R³)—OR³, C—SR³, N—R³, N—OR³ or N—N(R³)—R³ at the same time; and/or (iii) not more than two of W¹, W², W³ and W⁴ represent C═U at the same time, and/or (iv) if two of W¹, W², W³ and W⁴ represent O and/or S than least one carbon atom is present between them, and/or (v) when one of W¹, W², W³ and W⁴ represent CH, N, C—R³ or C—R⁴, C—N(R³)—R³, C—N(R³)—N(R³)—R³, C—N(R⁴)—N(R³)—R³, C—N(R³)—OR³, C—OR³, C—SR³, N—R³, N—OR³, N—N(R³)—R³ a double bond is formed within the condensed ring; and
U represents CH₂, O, S, or N—R³ or N—R⁴;
R³ each independently represents hydrogen; hydroxy; thiol; amino; cyano; formyl; halogen; nitro; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₁₂ alkynyl, C₃₋₈ cycloalkyl, C₄₋₁₂ (total carbon number) alkylcyclo-alkyl, C₄₋₁₂ (total carbon number) cycloalkylalkyl, C₁₋₆haloalkyl, C₁₋₆ alkylcarbonyl, C₁₋₆ alkyl-carbonyl-C₁₋₆ alkyl, C₁₋₆ alkylcarbonyl-C₁₋₆ alkylcarbonyl, C₁₋₆haloalkylcarbonyl, C₁₋₆ alkoxy-carbonyl, C₁₋₆ alkylsulfenylcarbonyl, C₁₋₆haloalkylsulfenylcarbonyl, aminocarbonyl, C₁₋₆ alkyl-aminocarbonyl, C₁₋₆haloalkylaminocarbonyl, C₁₋₆ hydroxyalkylaminocarbonyl, C₂₋₁₂ (total carbon number) dialkylamino-carbonyl, C₂₋₆ (total carbon number) di(haloalkyl)aminocarbonyl, C₂₋₆ alkenylaminocarbonyl, C₂₋₆ alkynylaminocarbonyl, C₁₋₆ alkyl-thiocarbonyl, C₃₋₆ cycloalkyl-carbonyl, C₄₋₁₂ (total carbon number) cycloalkylalkyl-carbonyl, C₃₋₆ cycloalkyl-thiocarbonyl, C₄₋₁₂ (total carbon number) cycloalkylalkyl-thiocarbonyl, C₁₋₆ haloalkyl-thiocarbonyl, C₁₋₆ alkyl-amino-thiocarbonyl, C₃₋₆ cycloalkylamino-carbonyl, C₄₋₁₂ (total carbon number) cycloalkylalkyl-amino-carbonyl, C₃₋₆ cycloalkylamino-thiocarbonyl, C₄₋₁₂ (total carbon number) cycloalkylalkyl-aminothiocarbonyl, C₁₋₆haloalkylamino-thiocarbonyl, C₂₋₁₂ (total carbon number) dialkylamino-1-thiocarbonyl, C₃₋₆ cycloalkyloxy-carbonyl, C₄₋₁₂ (total carbon number) cycloalkylalkyl-oxy-carbonyl, C₁₋₆haloalkoxy-carbonyl, C₁₋₆ alkylsulfonyl, C₁₋₆ haloalkylsulfonyl, phenylsulfonyl, R⁴—C₁₋₆ alkyl, R⁴-carbonyl, R⁴-thiocarbonyl, R⁴—C₁₋₆ alkylcarbonyl, R⁴—C₁₋₆alkyl-thiocarbonyl, R⁴-oxycarbonyl, R⁴—C₁₋₆ alkyloxy-carbonyl, R⁴-aminocarbonyl, R⁴-amino-thiocarbonyl, R⁴—C₁₋₆ alkylamino-carbonyl, or R⁴—C₁₋₆alkylamino-thiocarbonyl; and
R⁴ represents phenyl or 5- or 6-membered saturated or unsaturated heterocyclic ring, preferably is selected among the groups R⁴-1 to R⁴-83

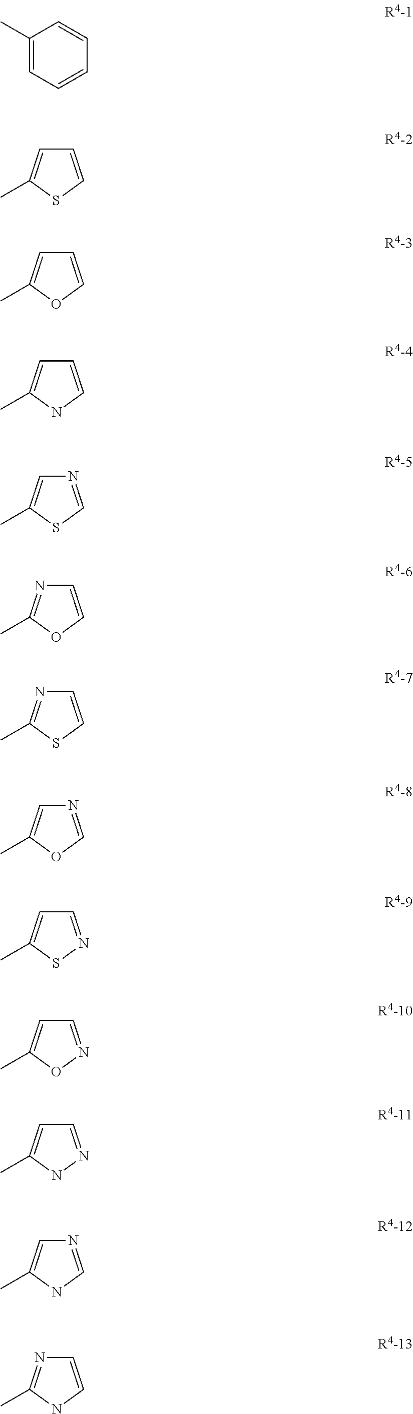

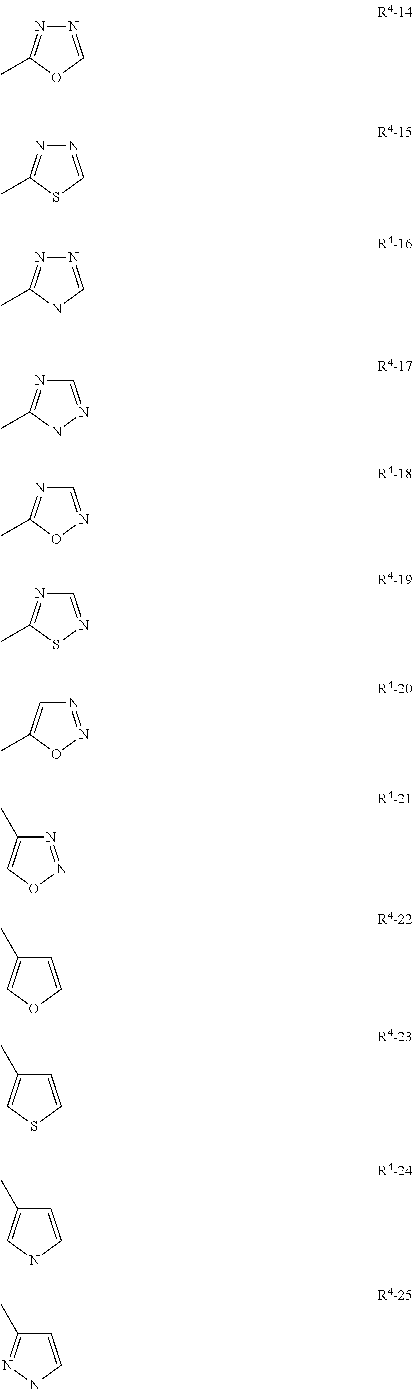

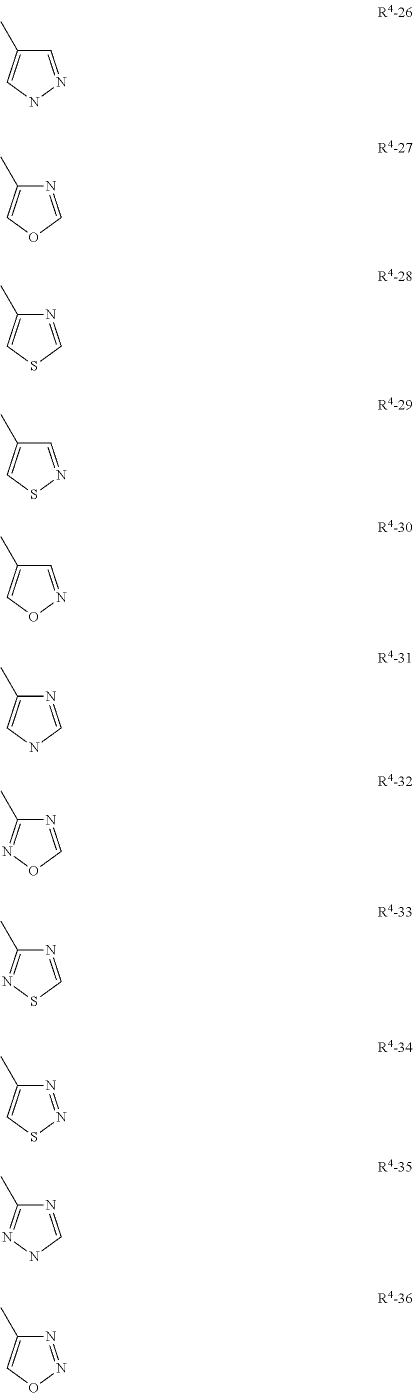

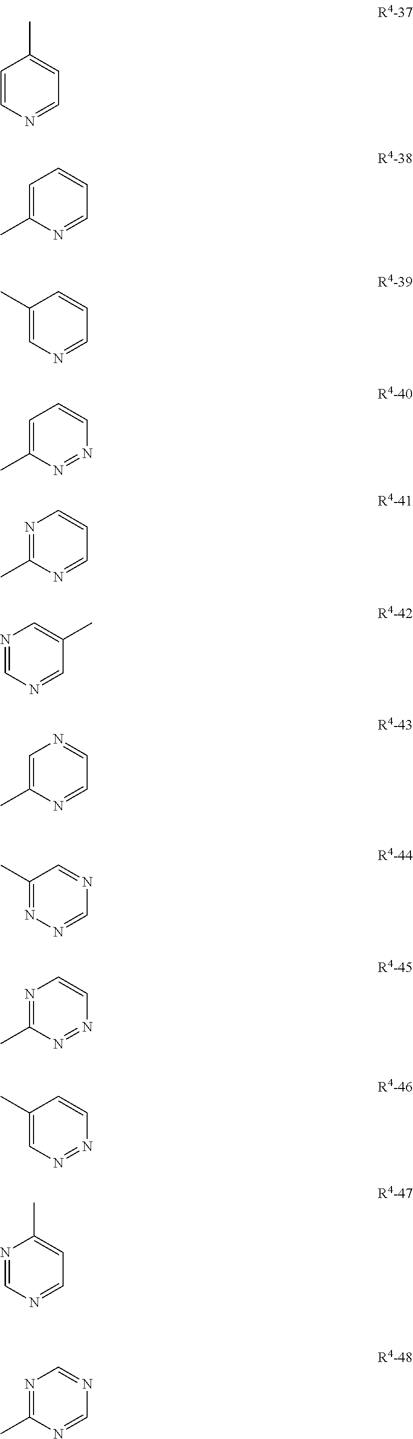

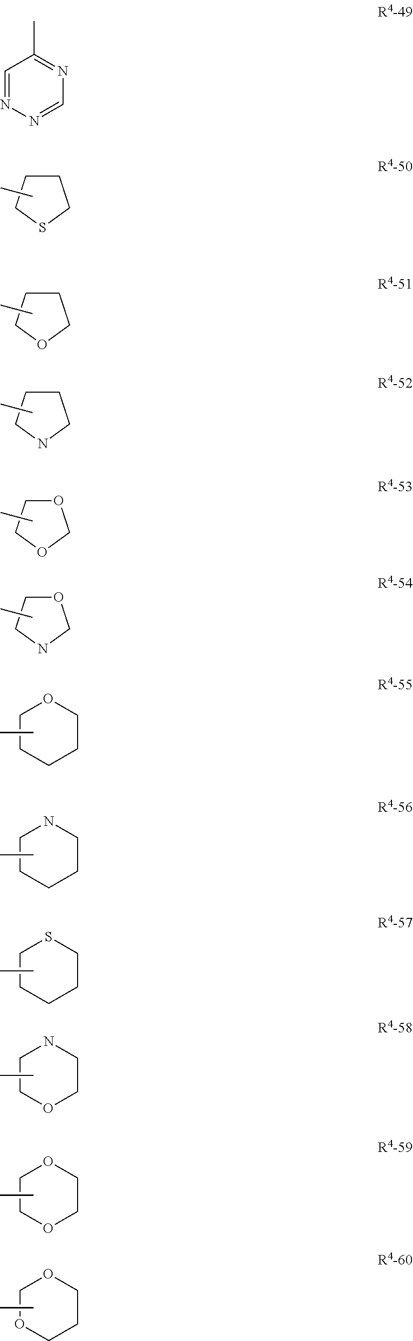

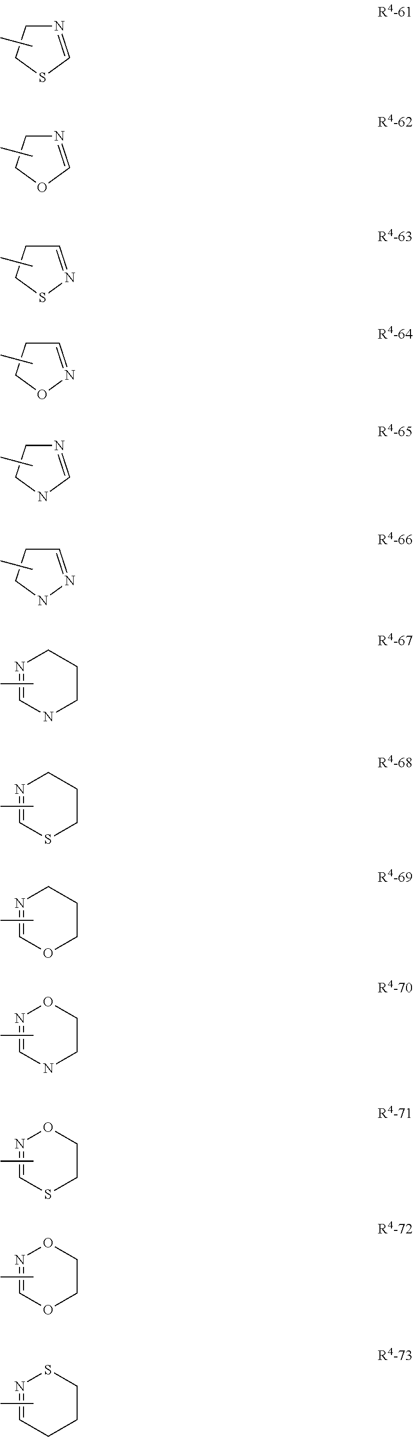

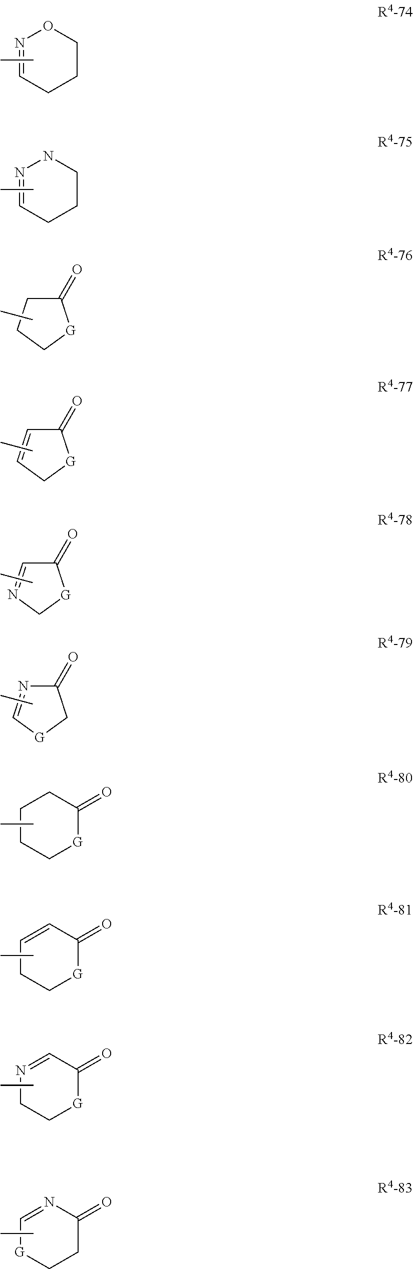

wherein
G represents O, S or N, and wherein each group R⁴-1 to R⁴-83 may be substituted with at least one group selected among hydrogen; halogen; cyano, nitro; C₁₋₆ alkyl, C₁₋₆ haloalkyl, C₃₋₆ cycloalkyl, C₃₋₆ halocycloalkyl, C₁₋₆ alkoxy, C₁₋₆ haloalkoxy, C₁₋₆ alkylsulfenyl, C₁₋₆ haloalkyl-sulfenyl, C₁₋₆ alkylsulfinyl, C₁₋₆ haloalkylsulfinyl, C₁₋₆ alkylsulfonyl, C₁₋₆ haloalkylsulfonyl, C₁₋₆ alkylamino, C₁₋₆ haloalkylamino, aminocarbonyl, C₁₋₆ alkylamino-carbonyl, C₂₋₁₂ dialkylamino-carbonyl, C₁₋₆ alkoxycarbonyl, phenyl, or pyridyl;
   preferably the group —W¹—W²—W³—W⁴— is selected among the 4-5- or 6-membered groups W-1 to W-580

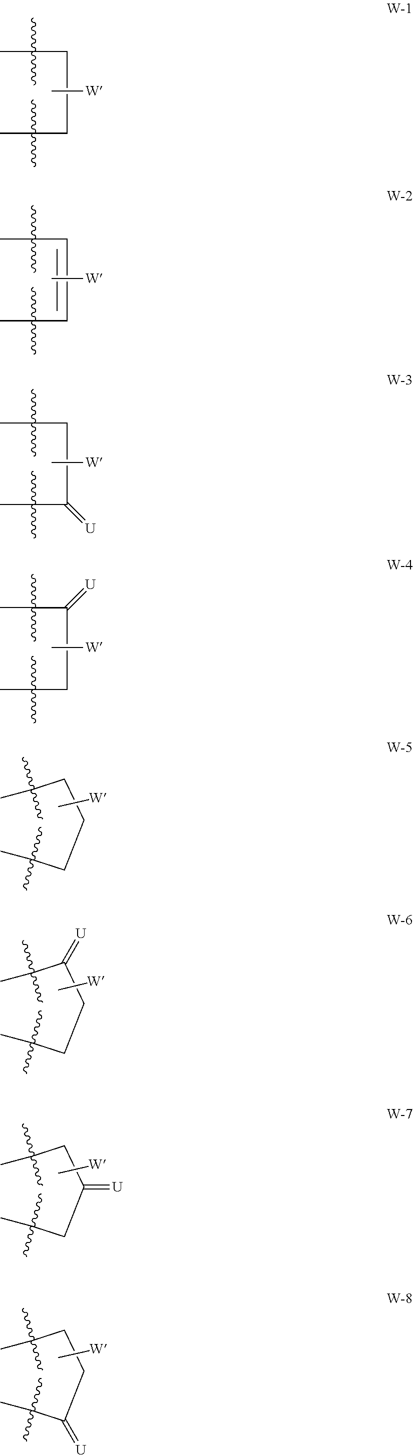

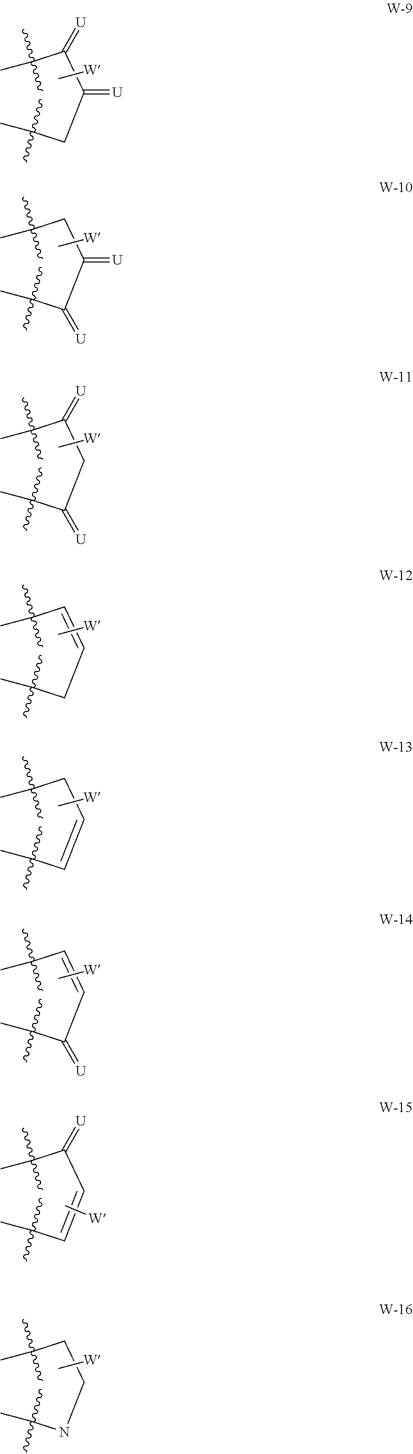

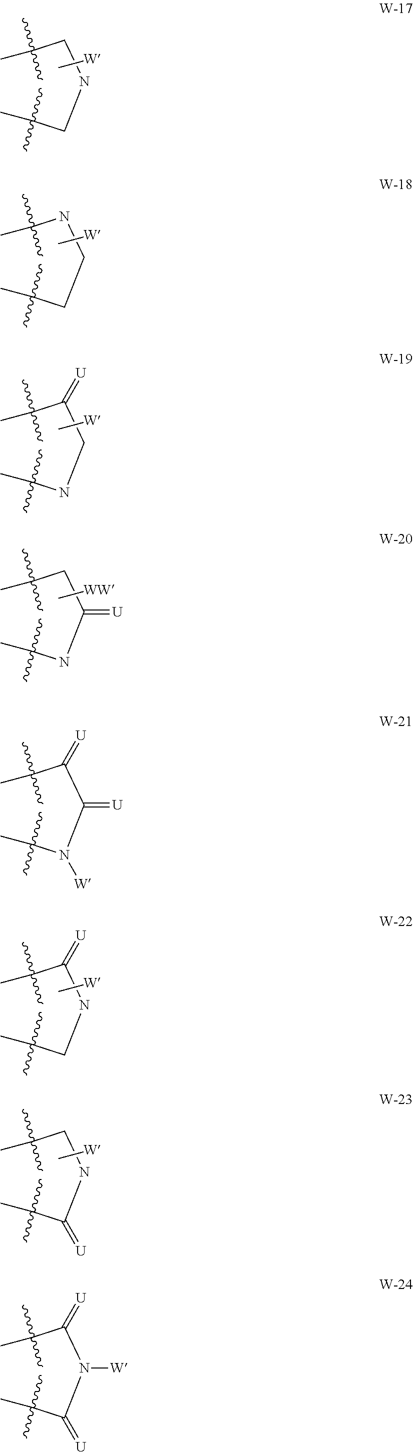

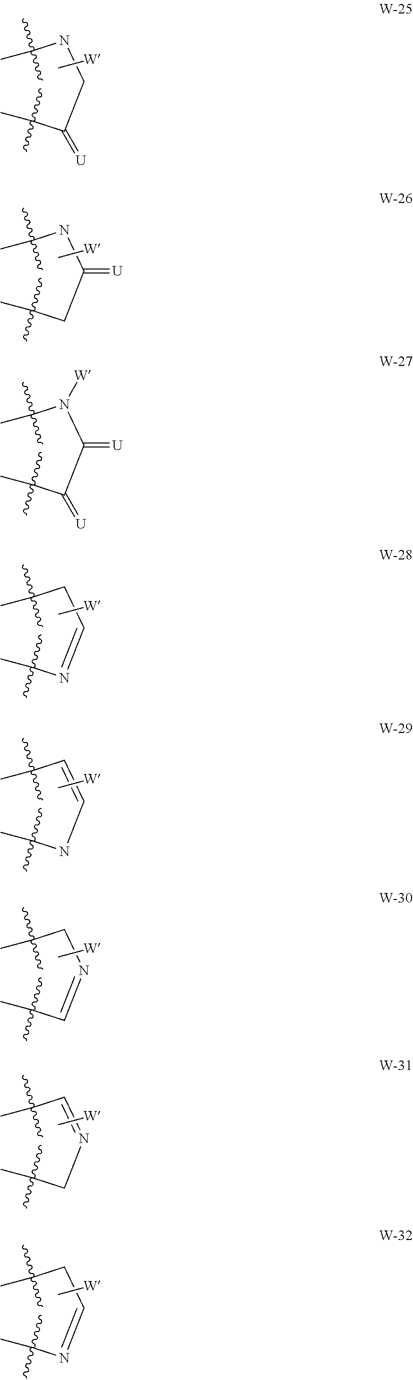

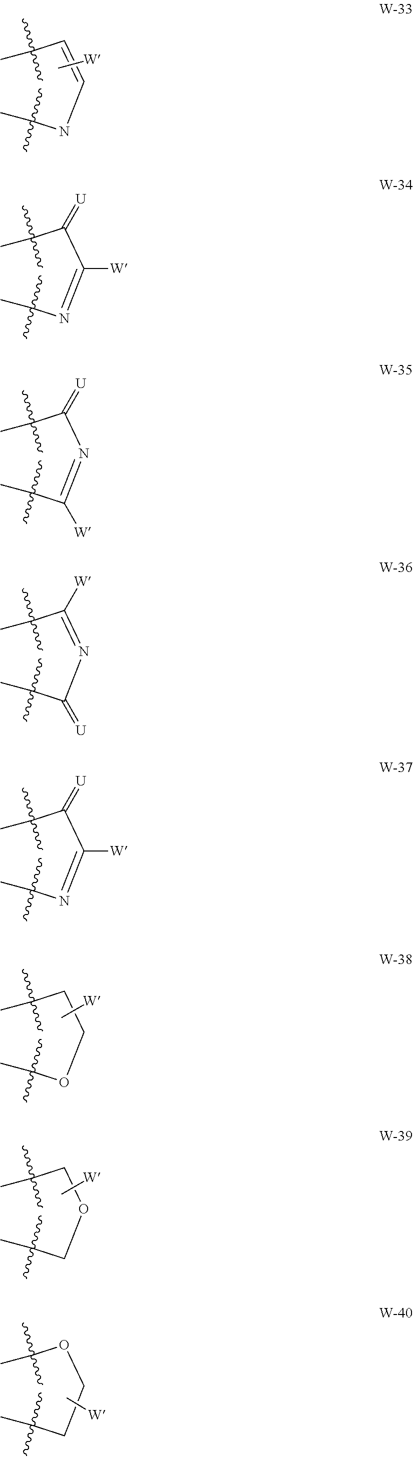

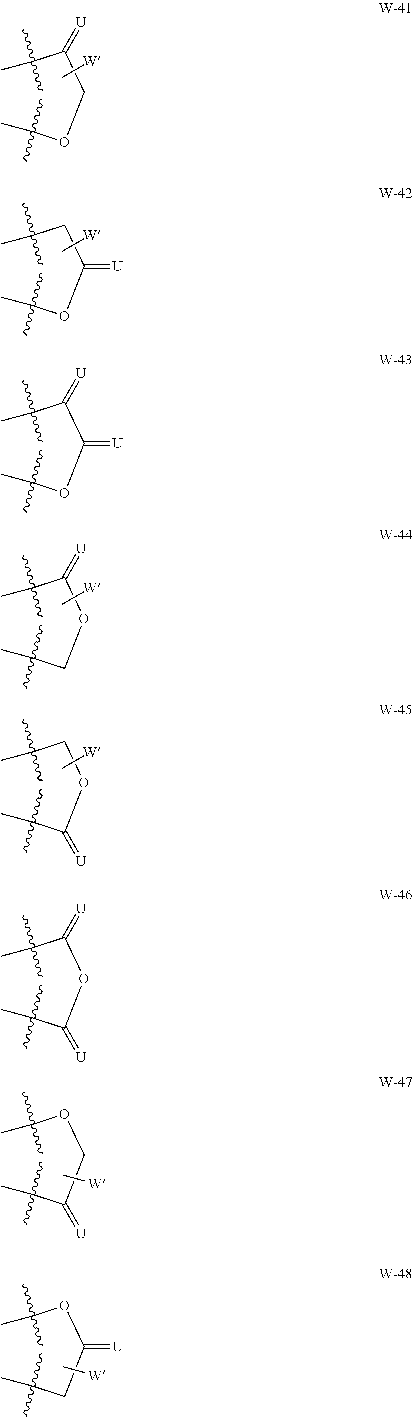

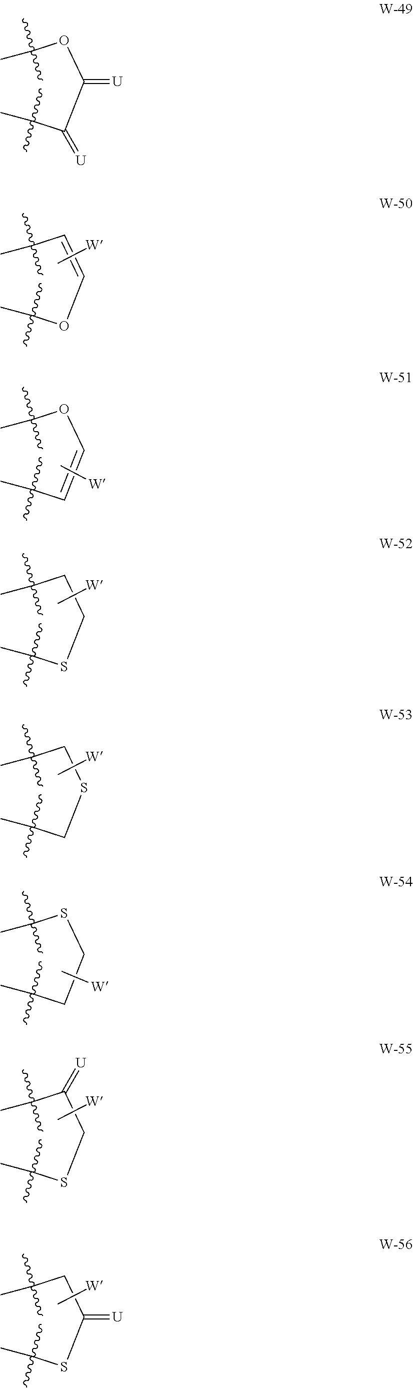

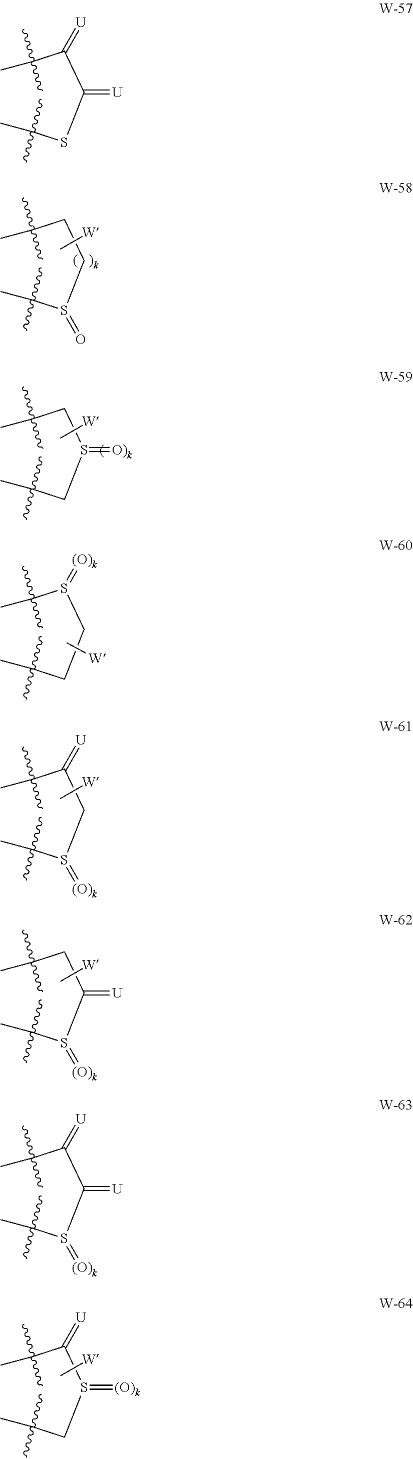

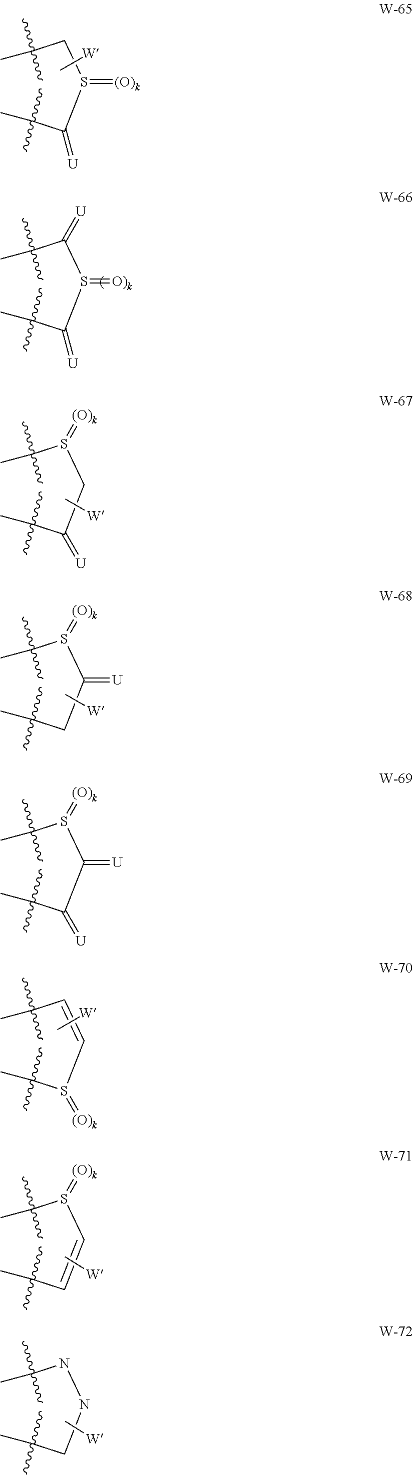

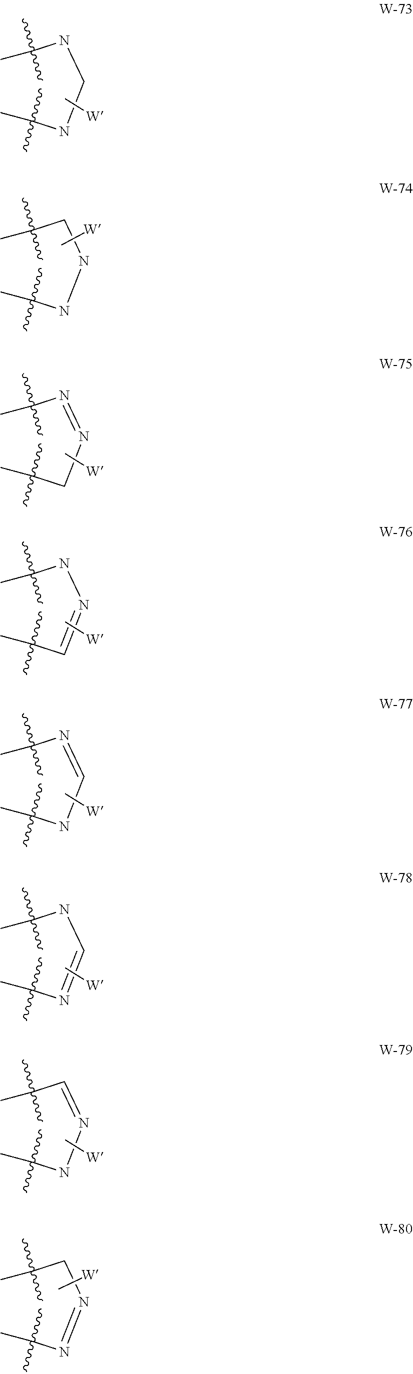

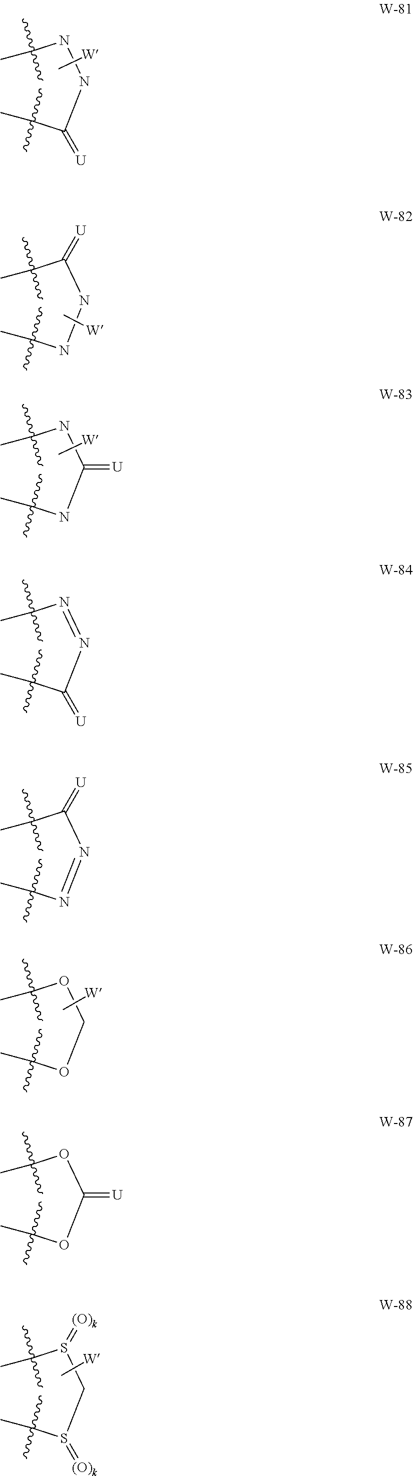

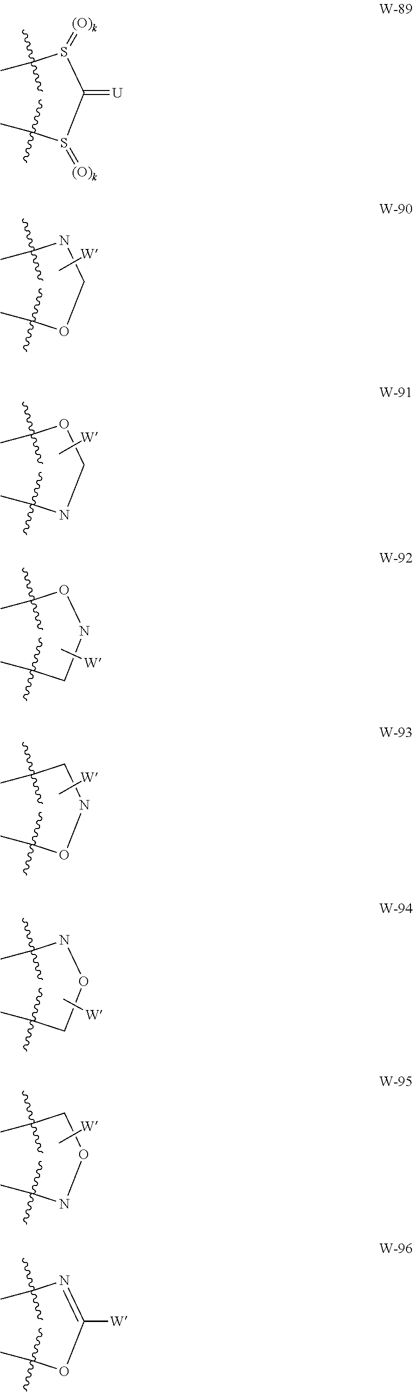

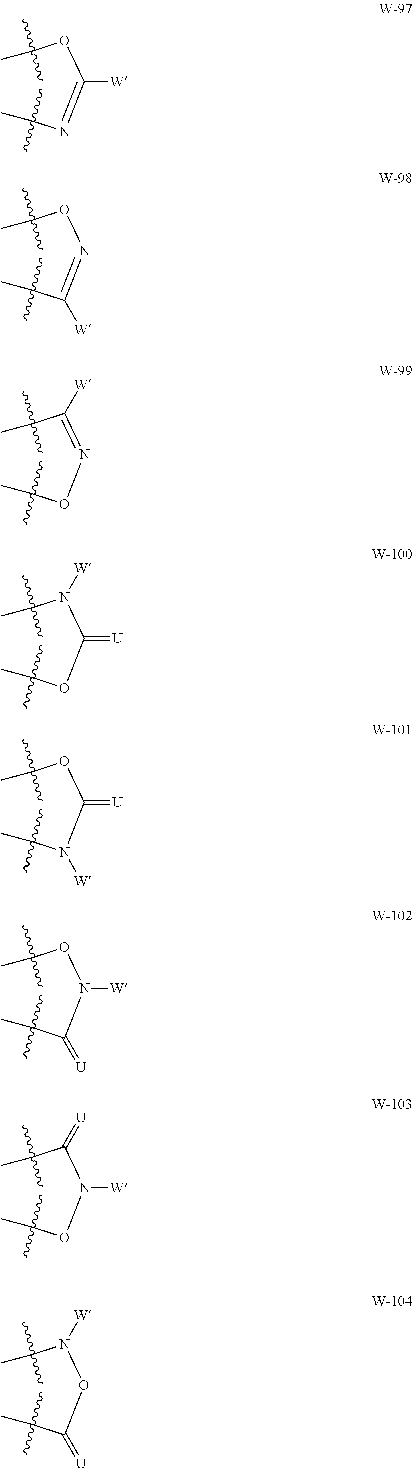

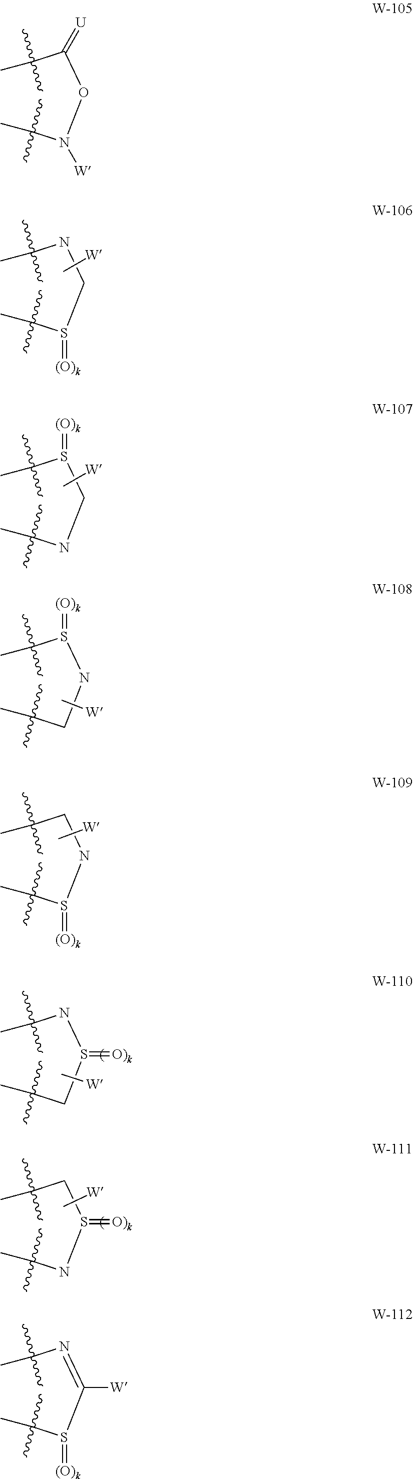

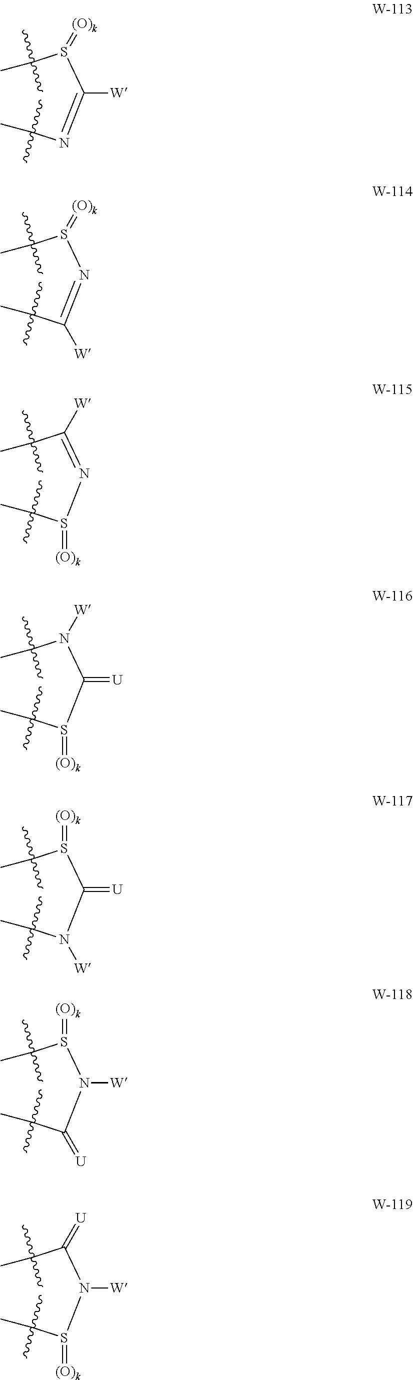

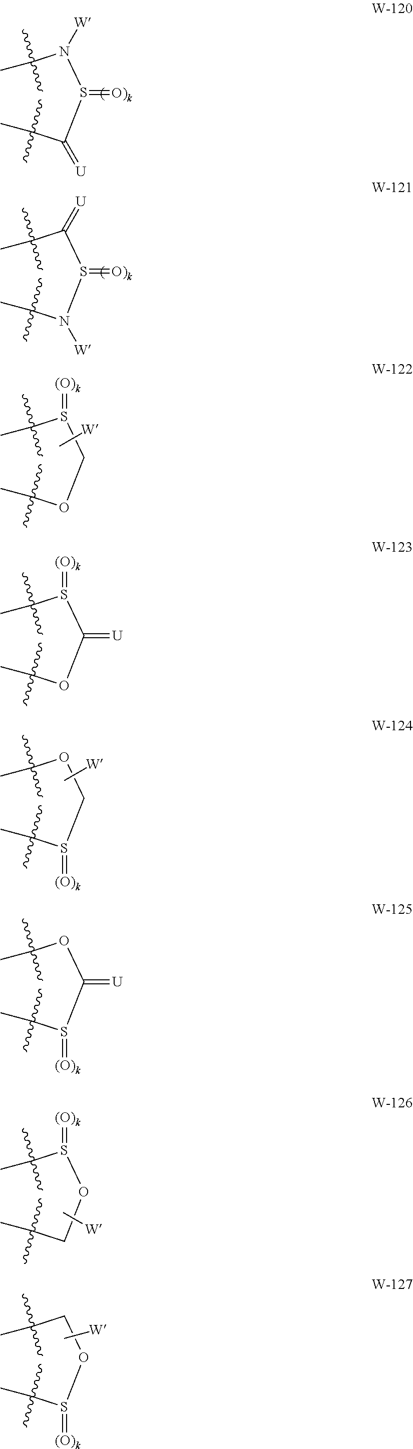

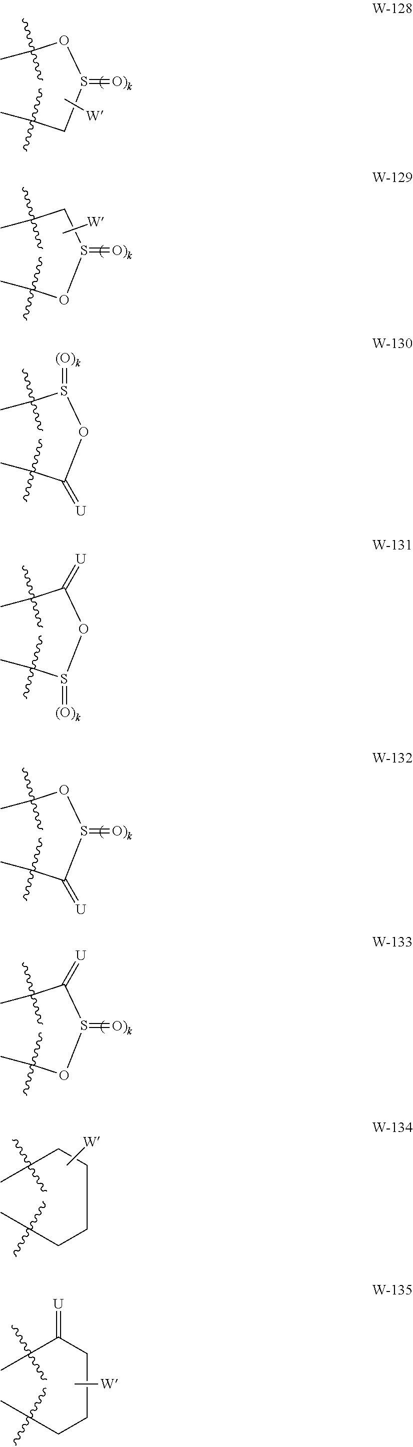

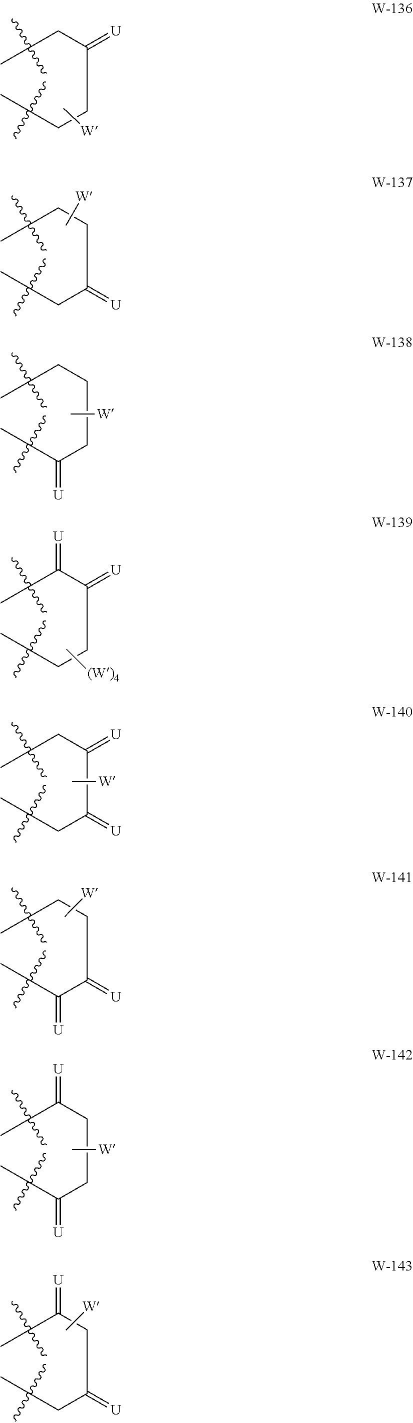

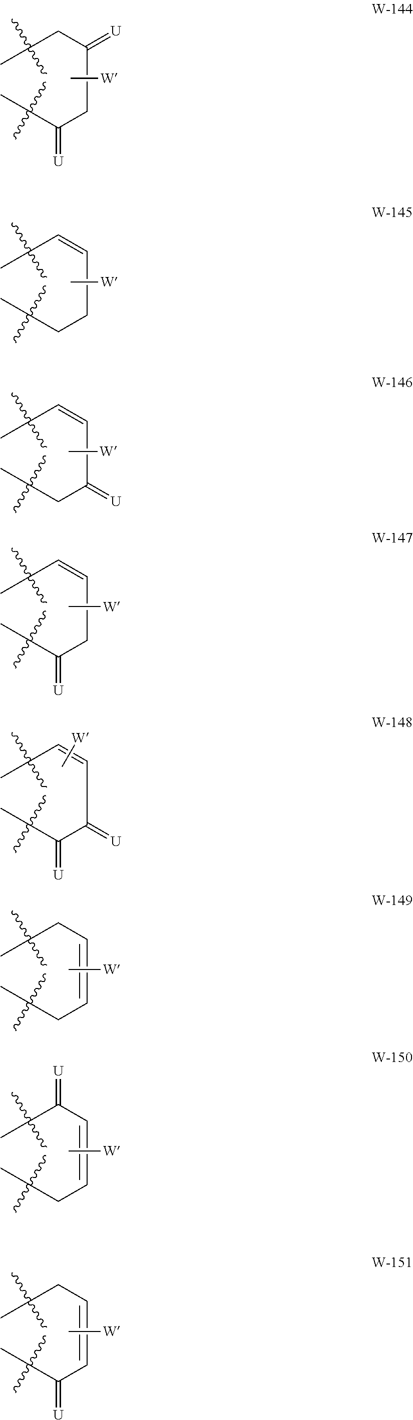

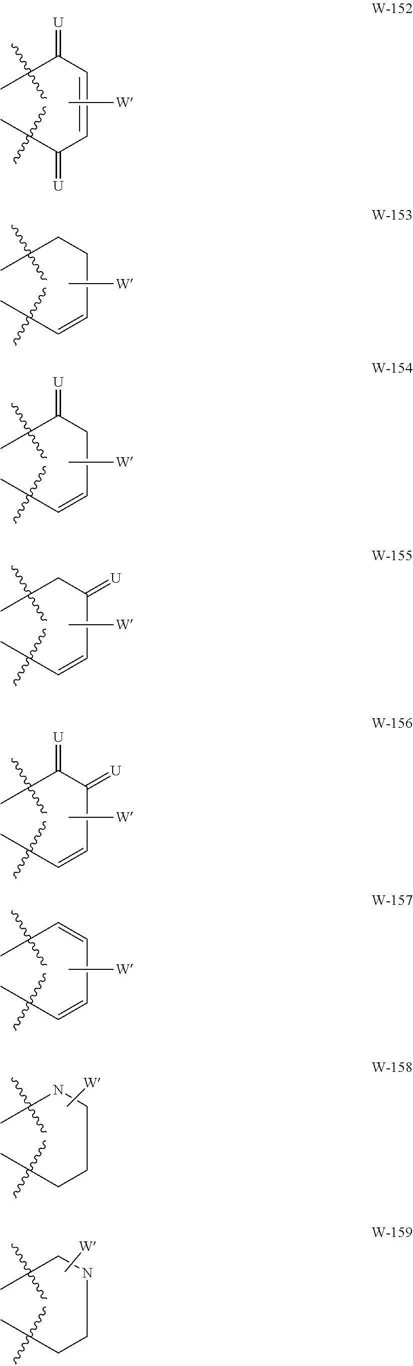

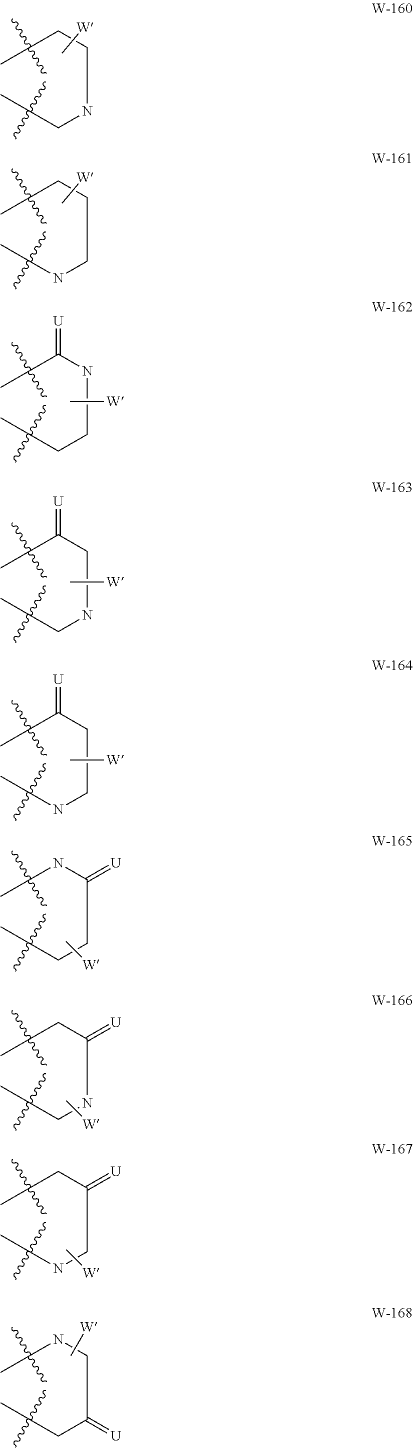

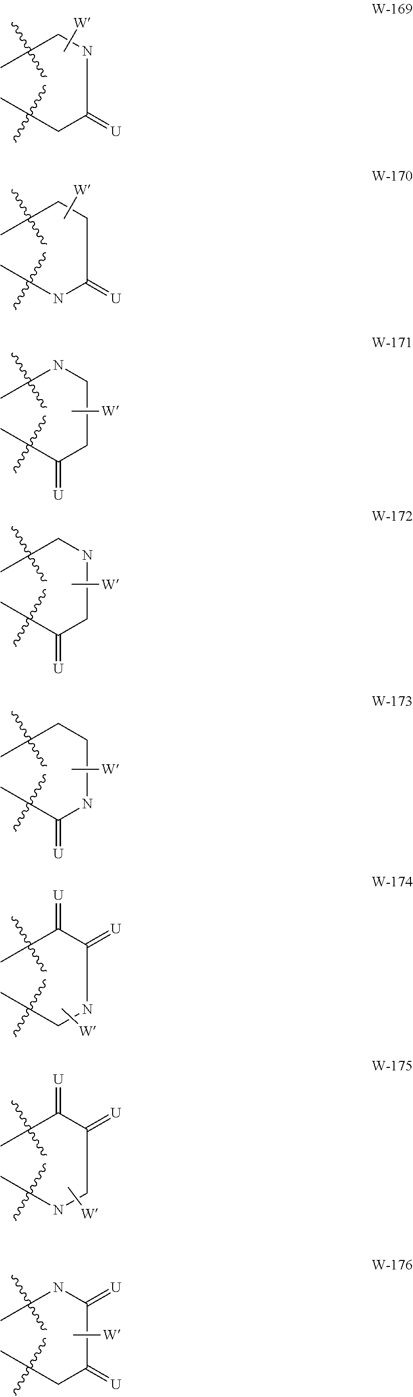

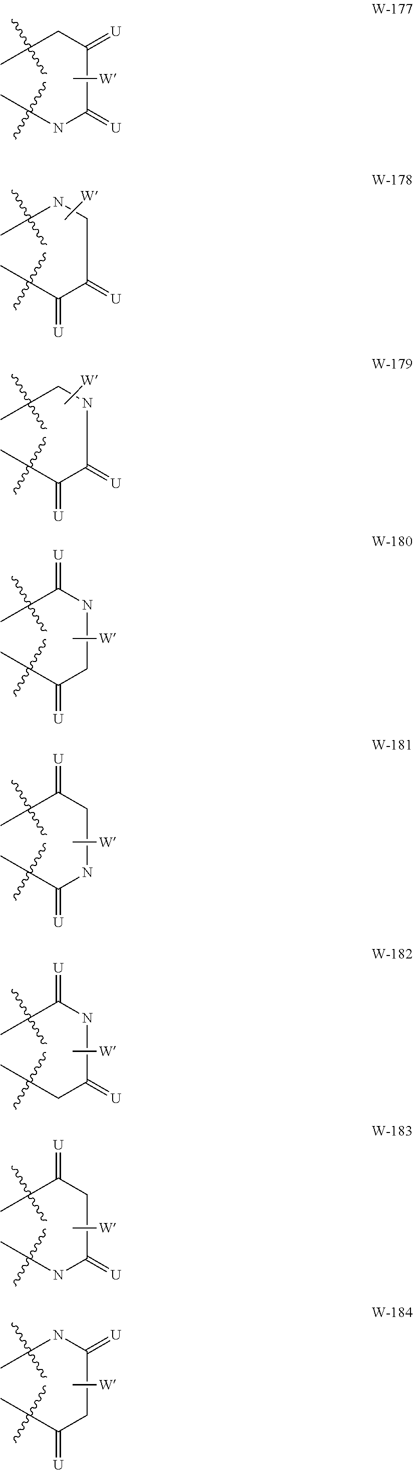

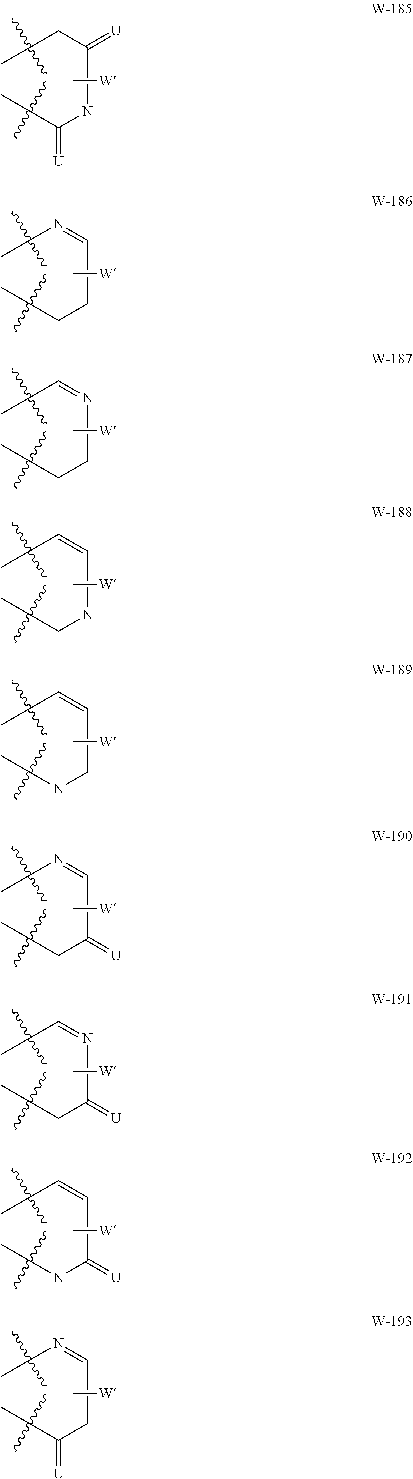

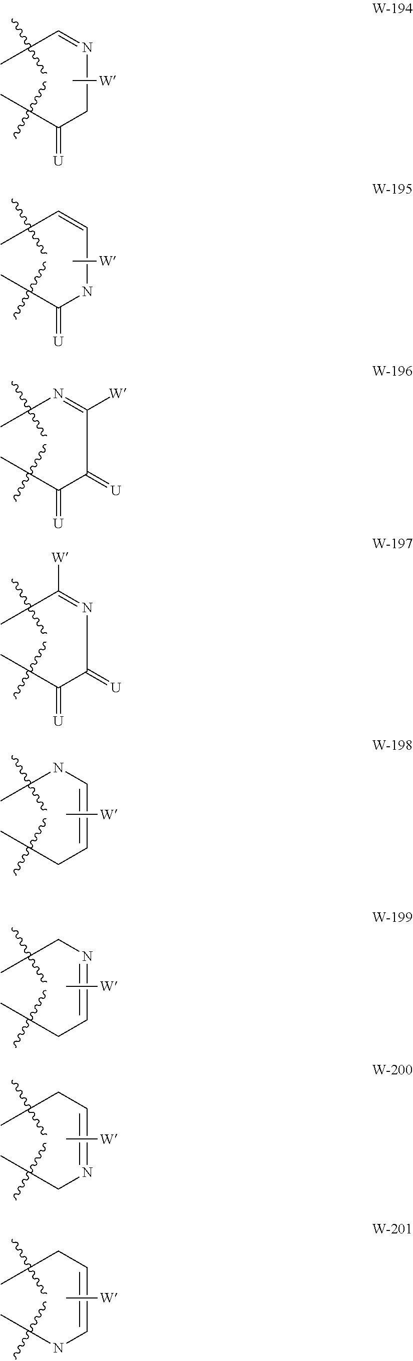

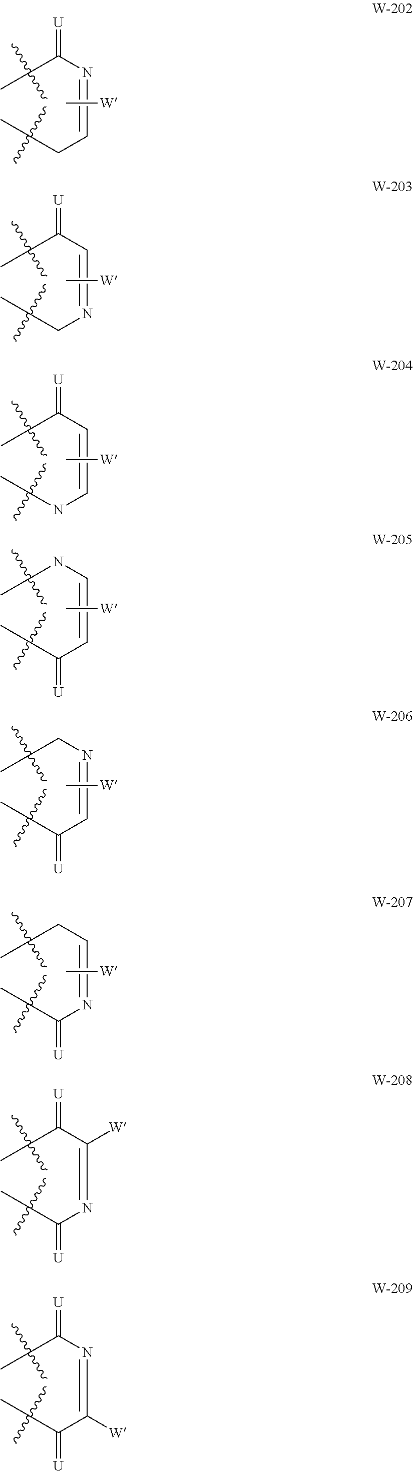

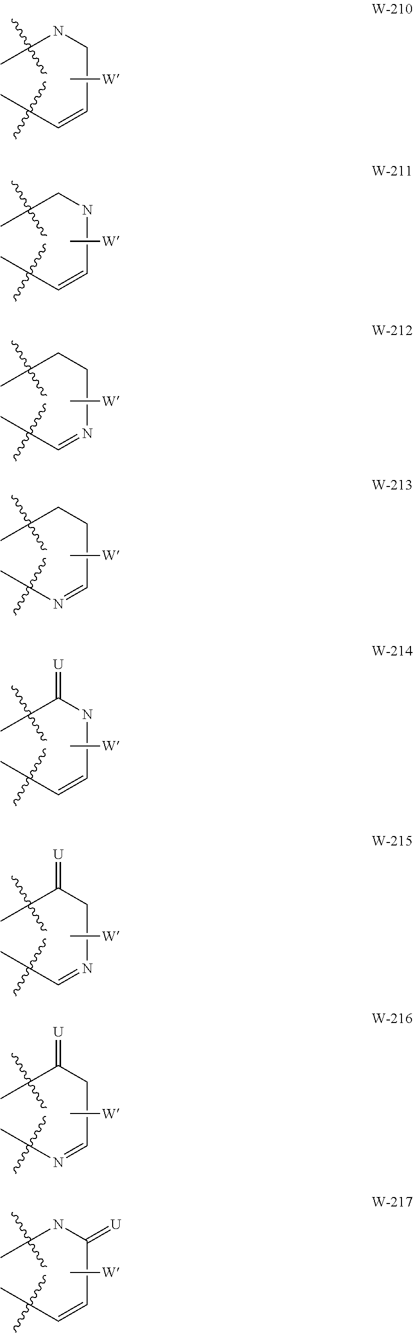

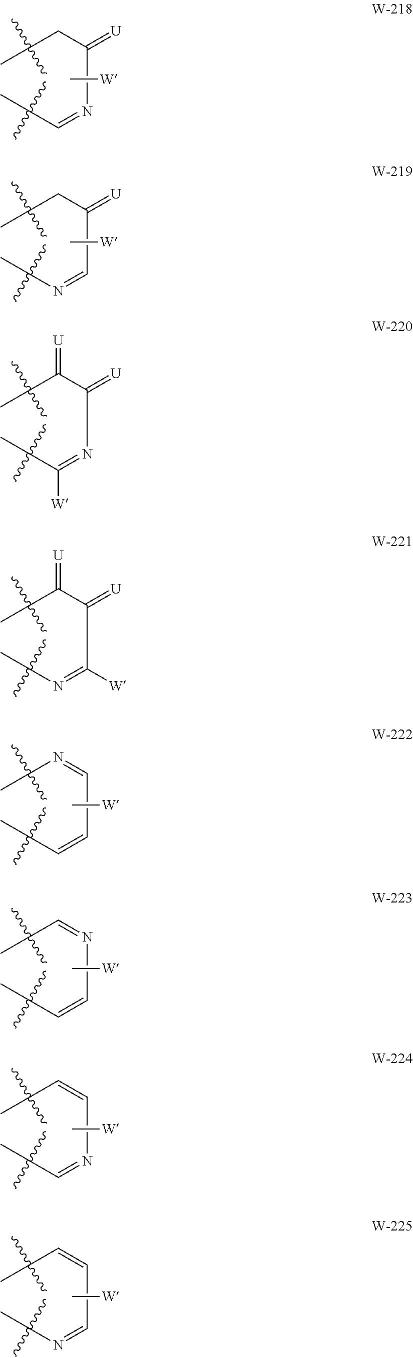

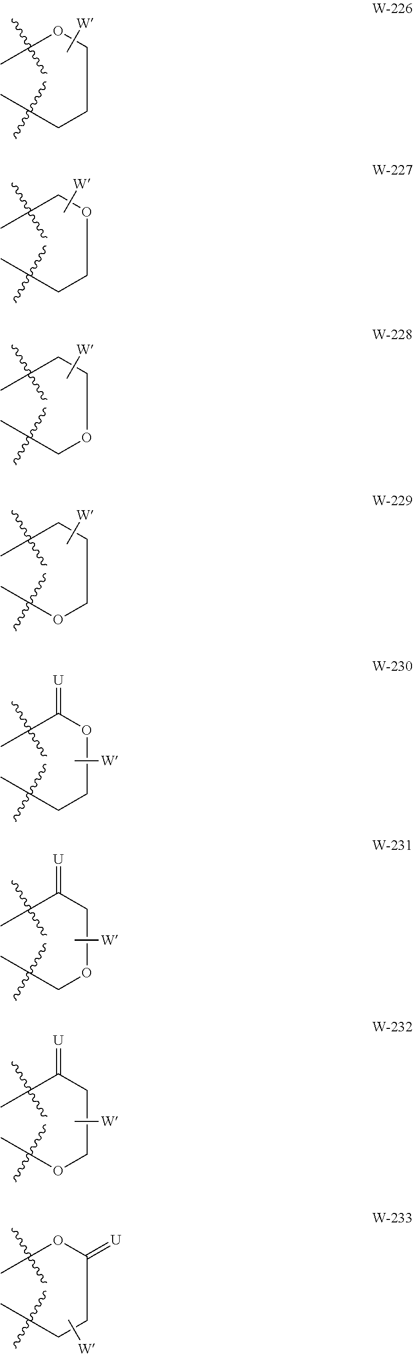

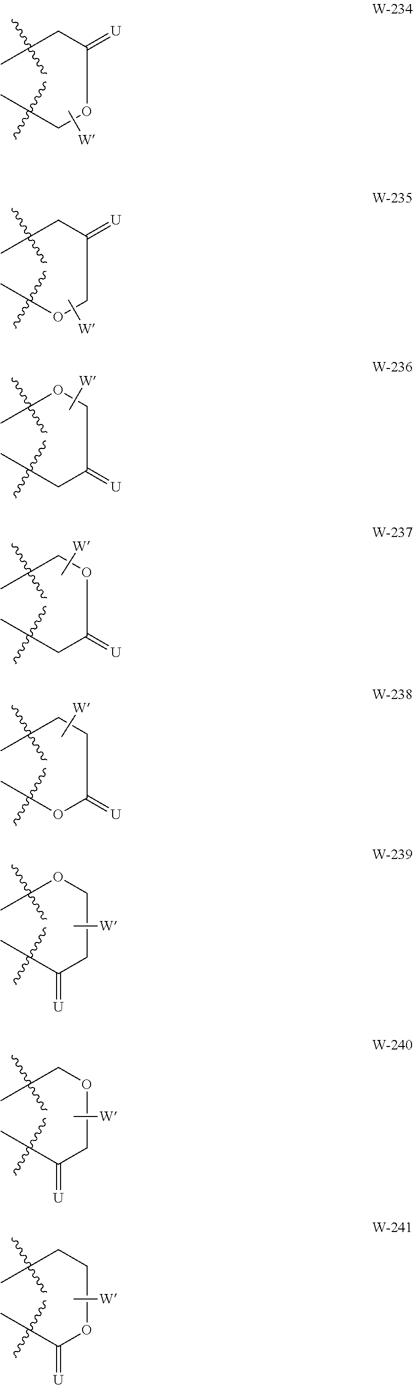

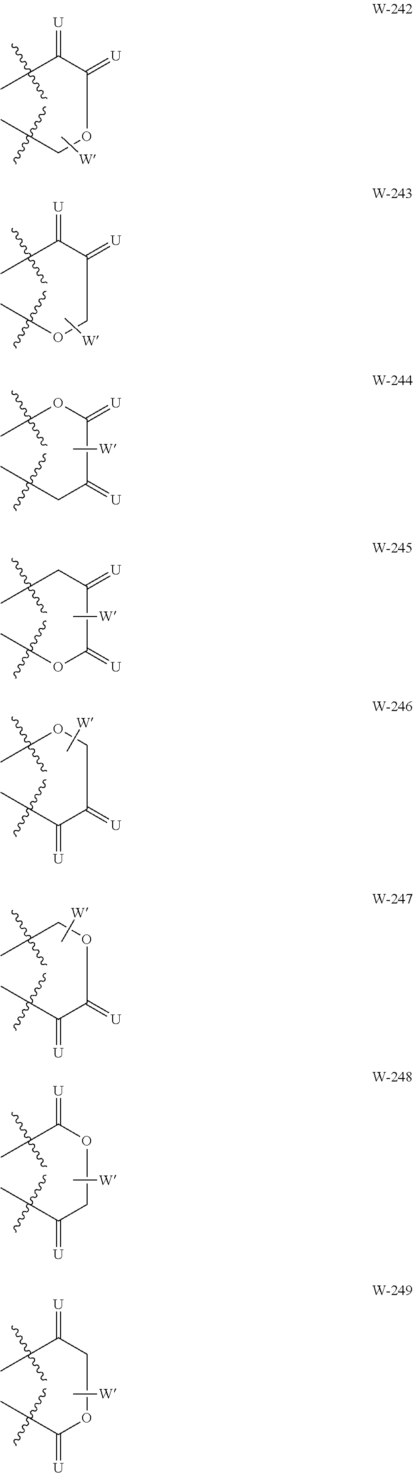

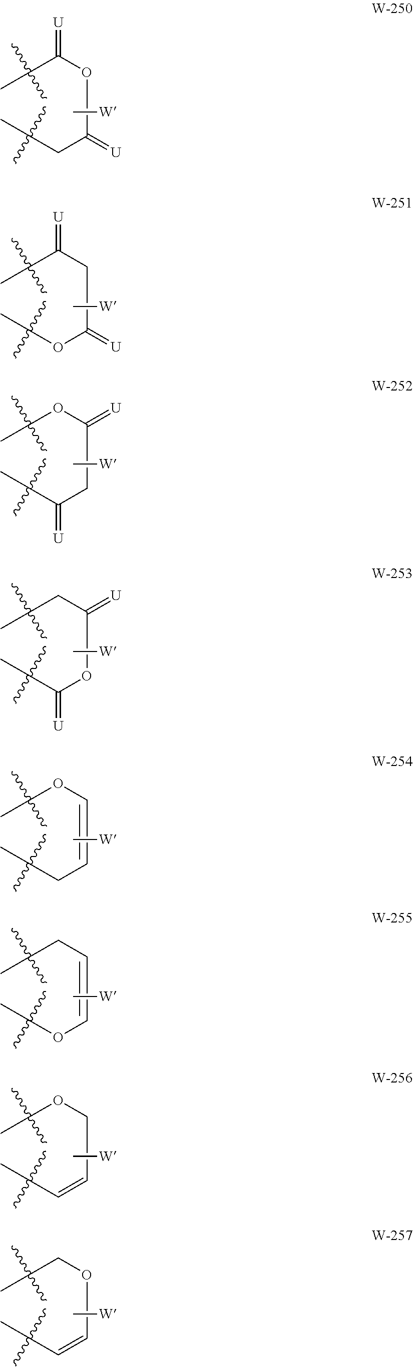

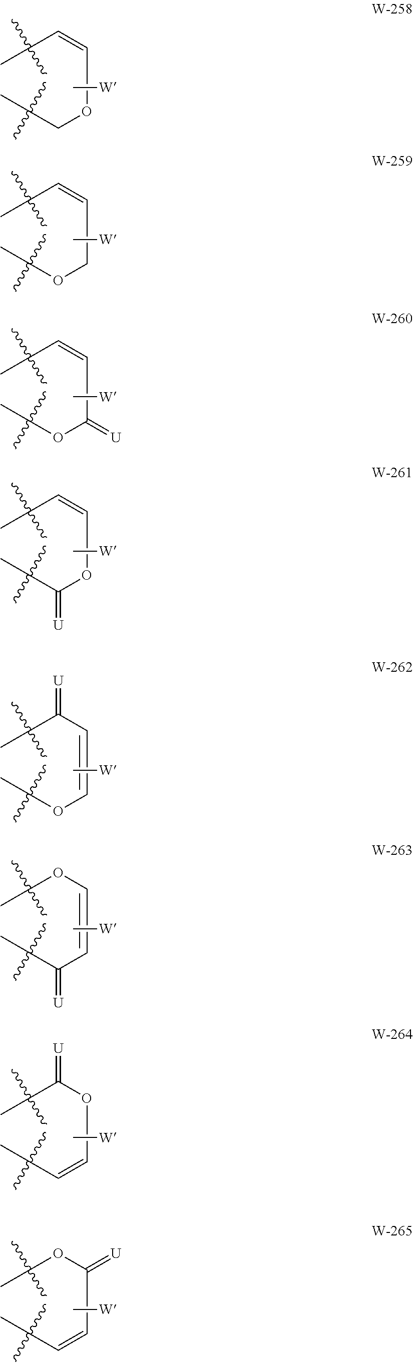

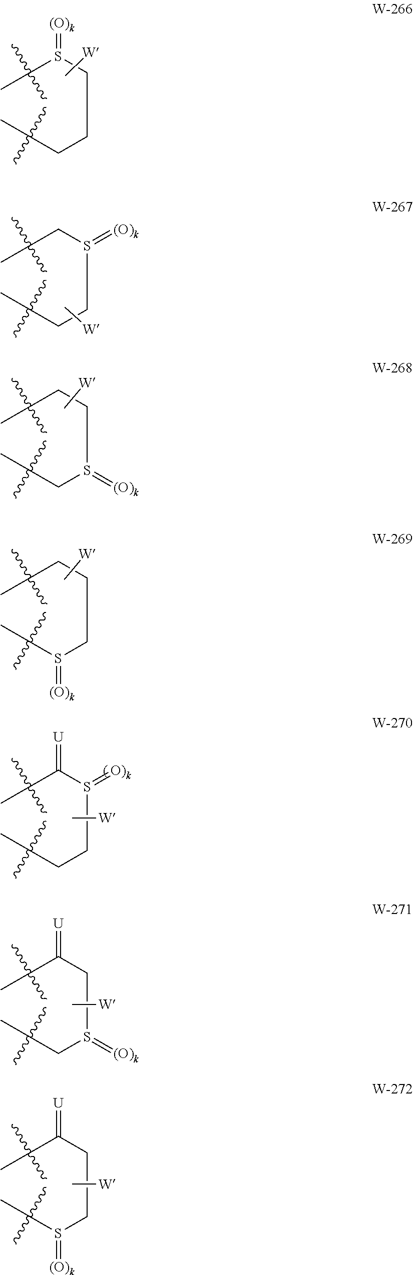

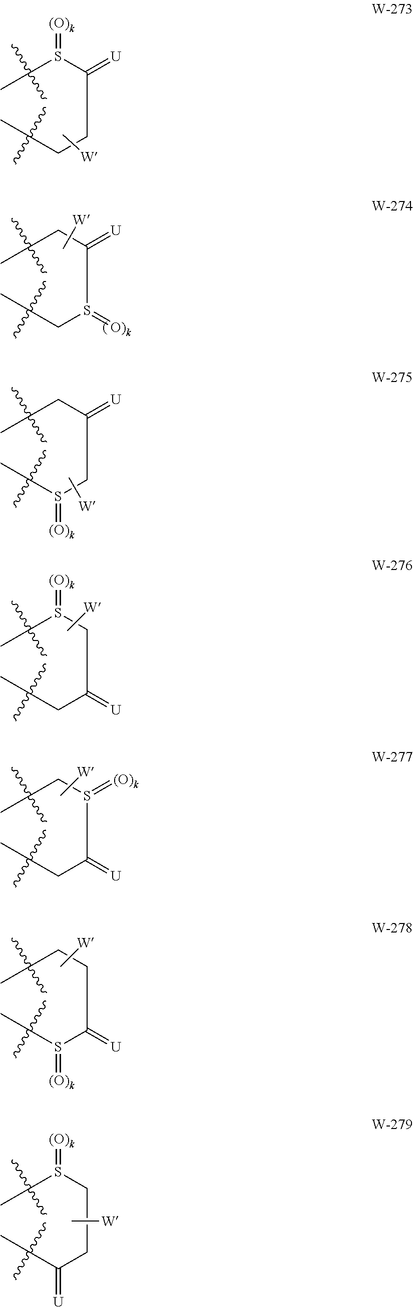

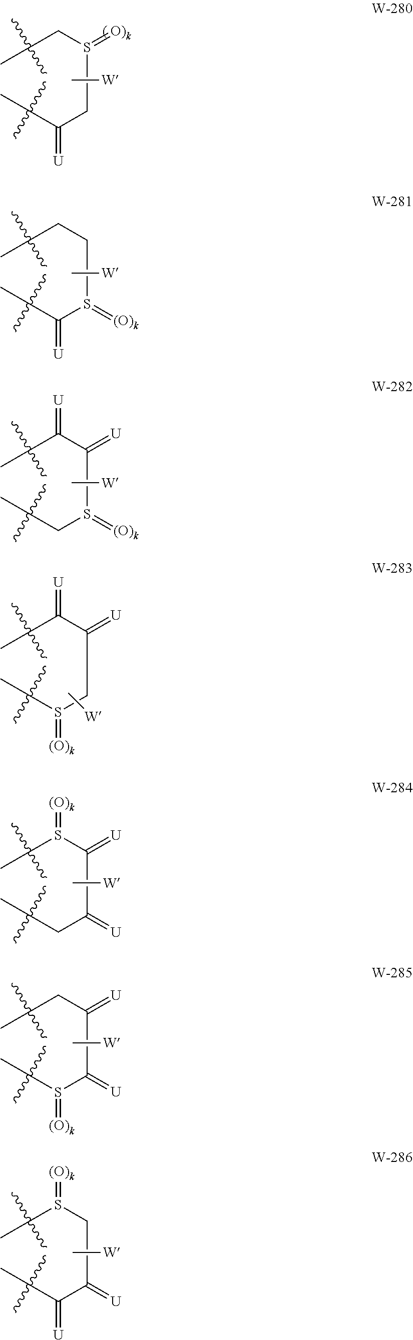

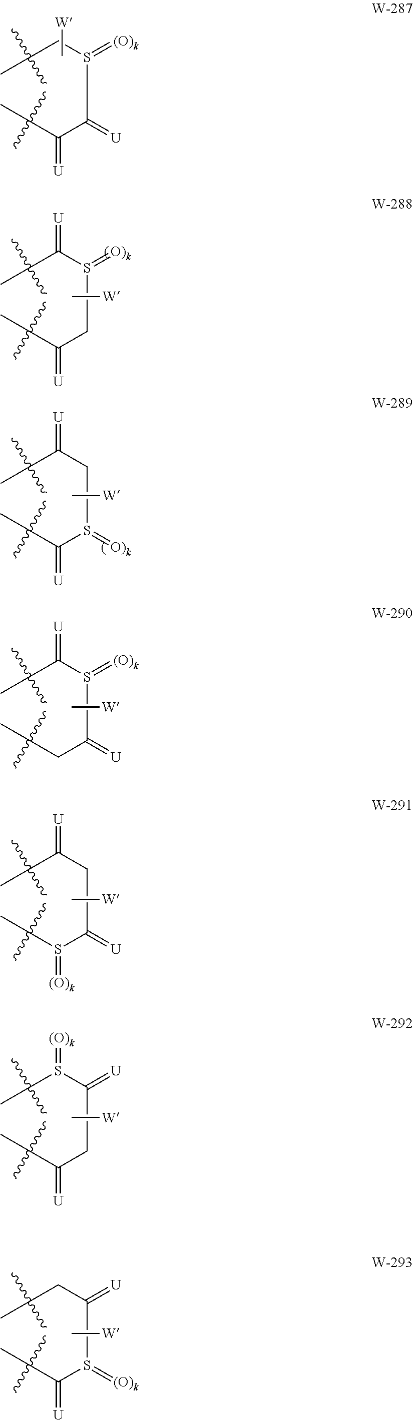

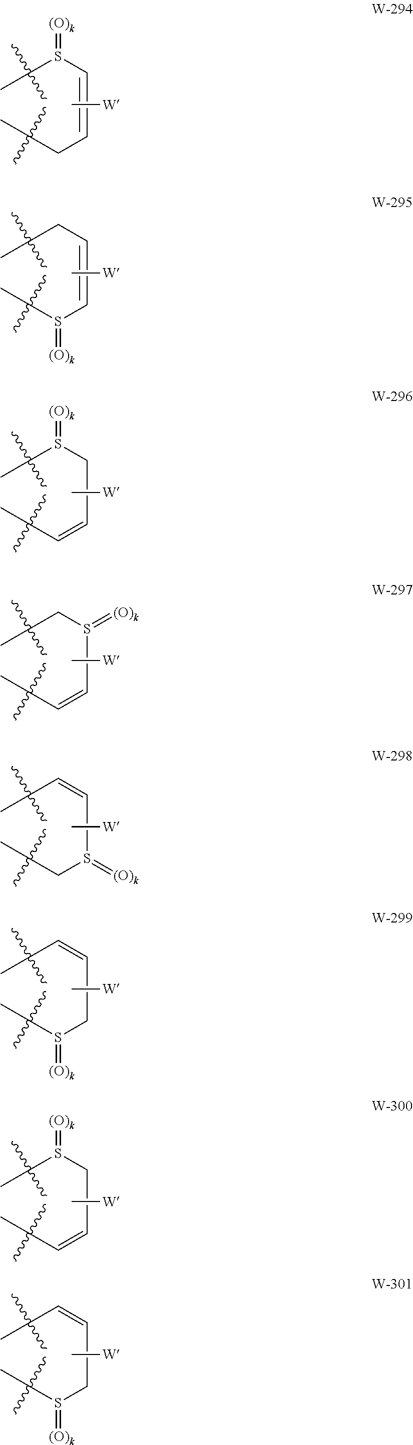

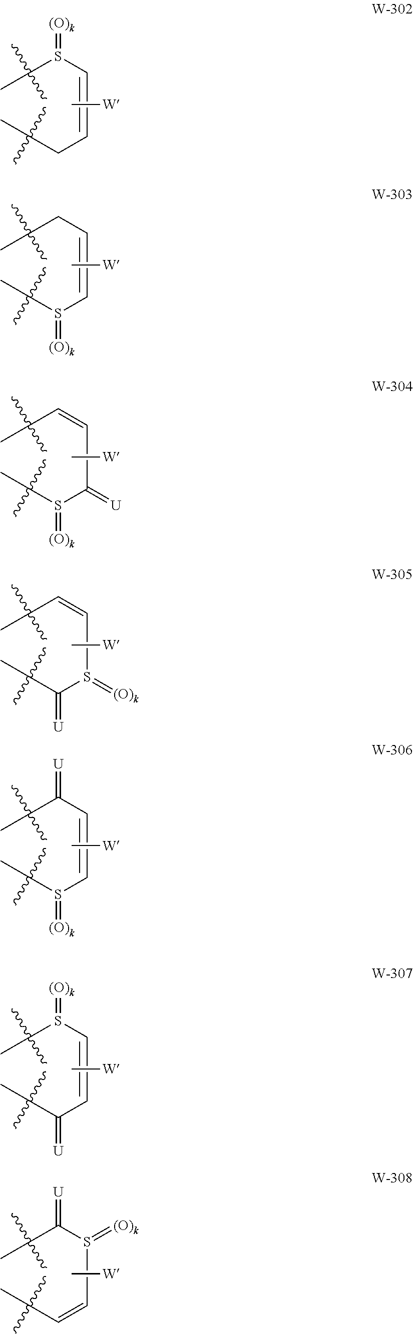

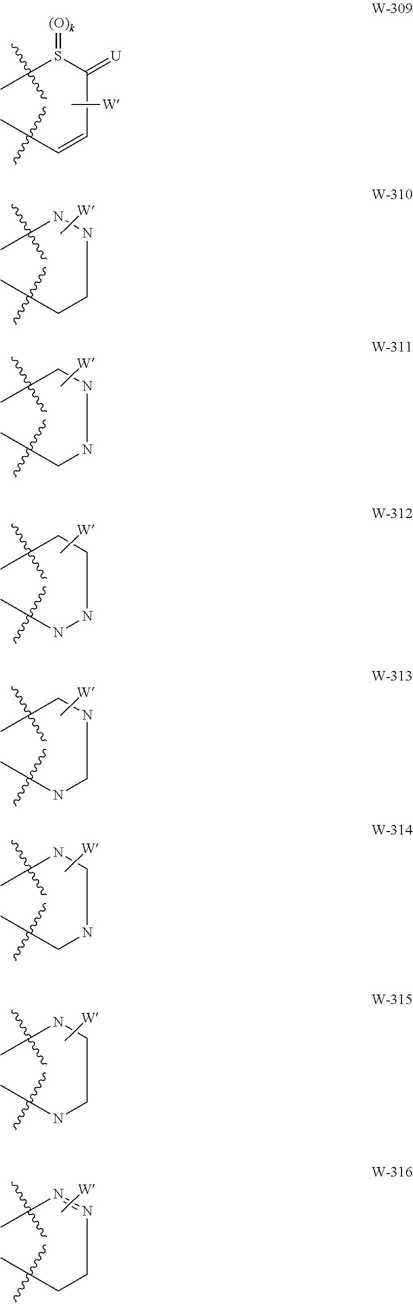

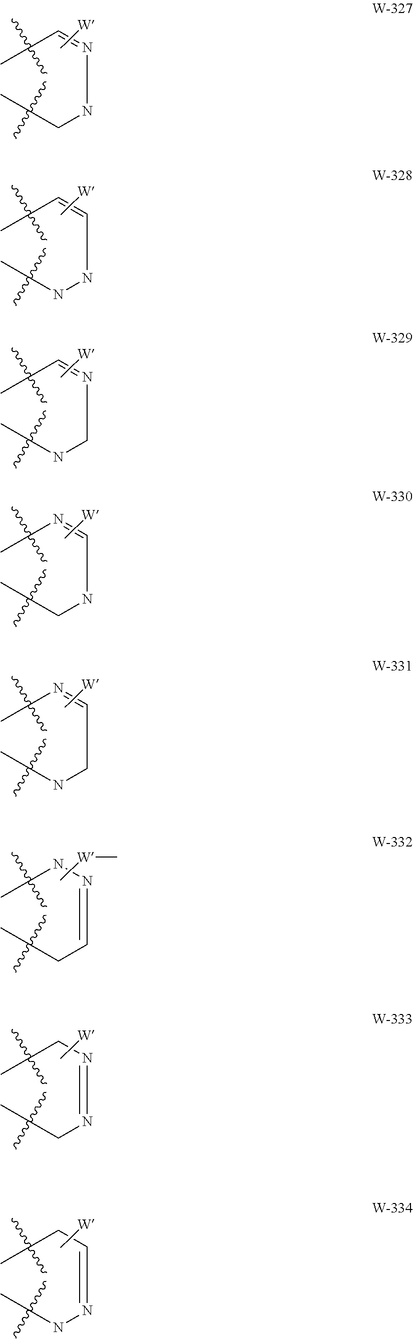

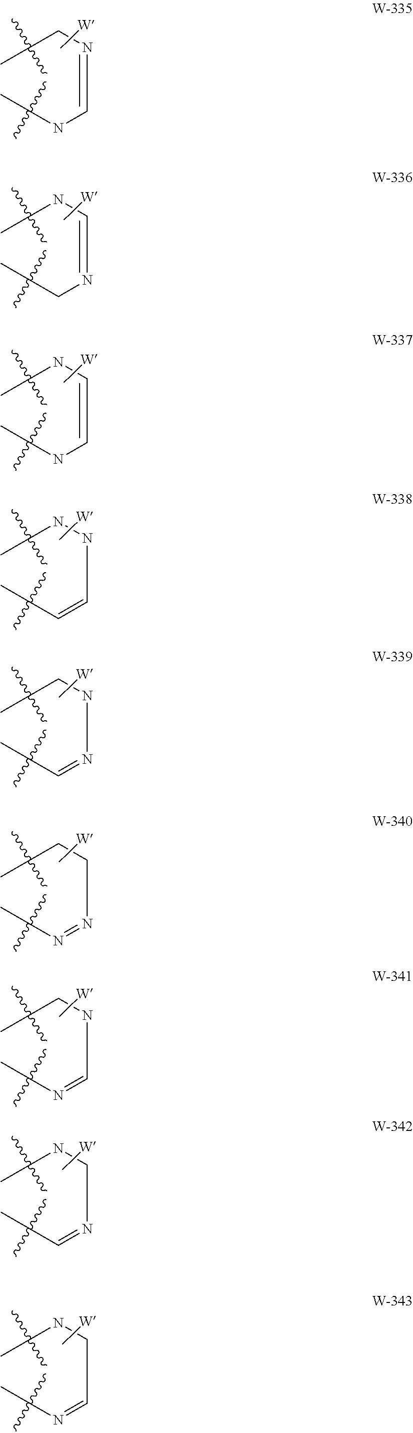

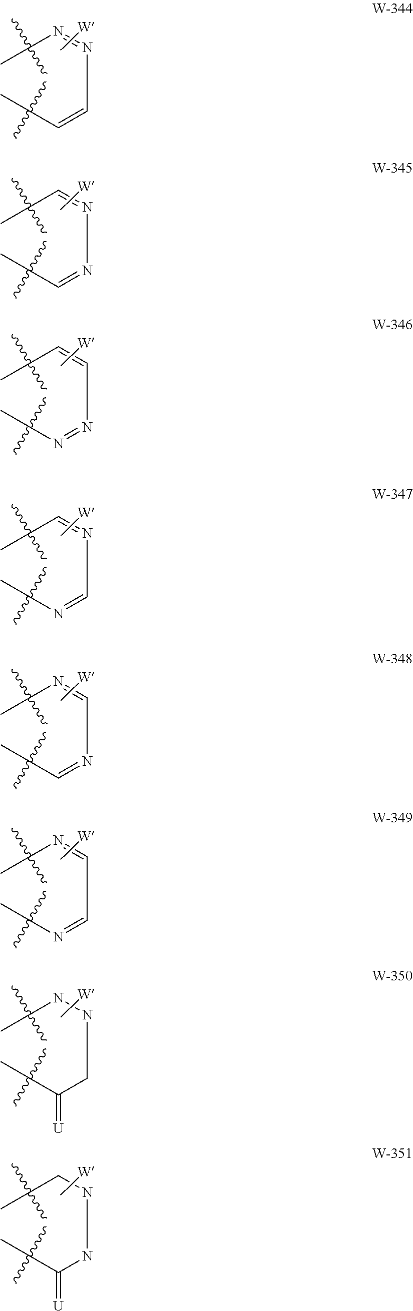

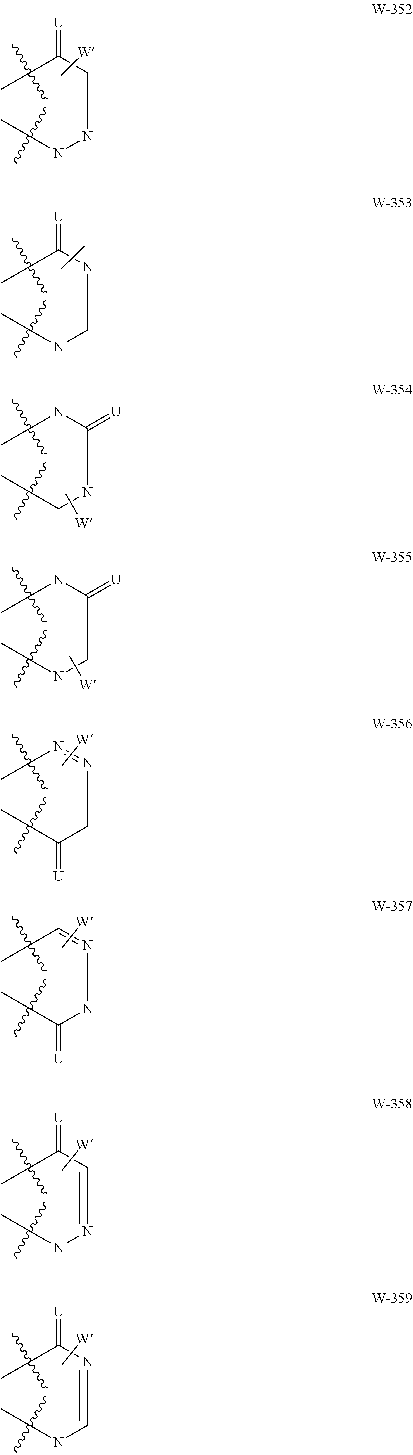

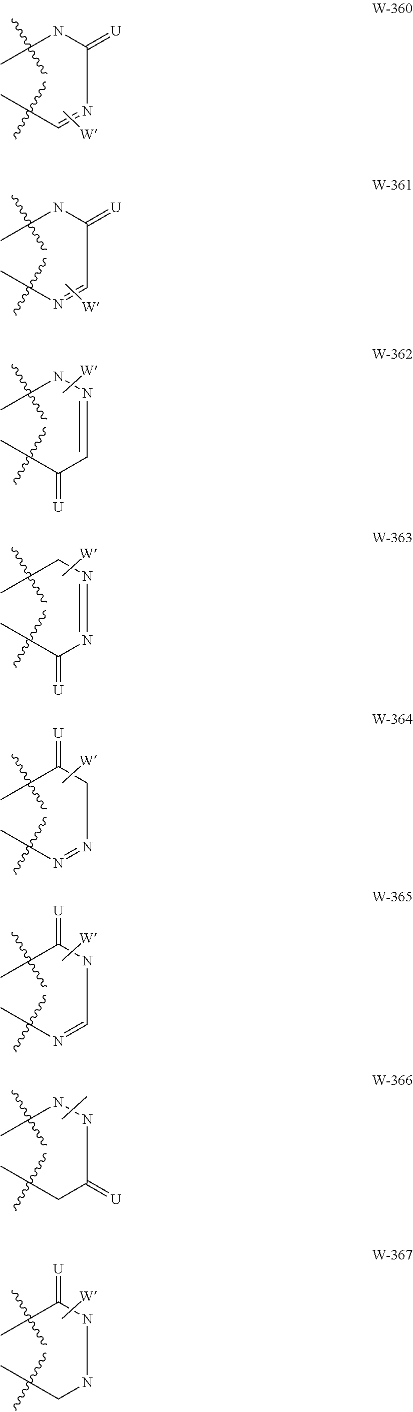

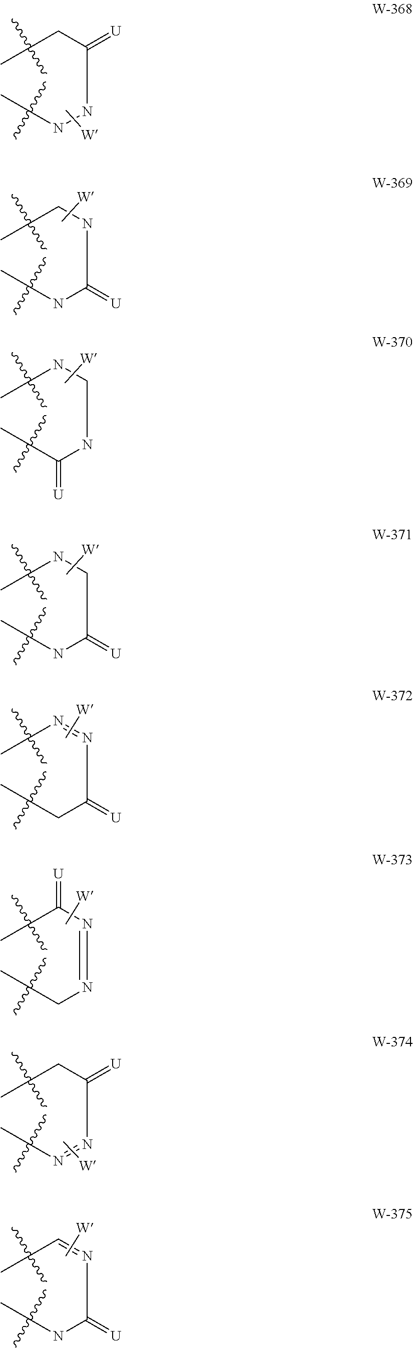

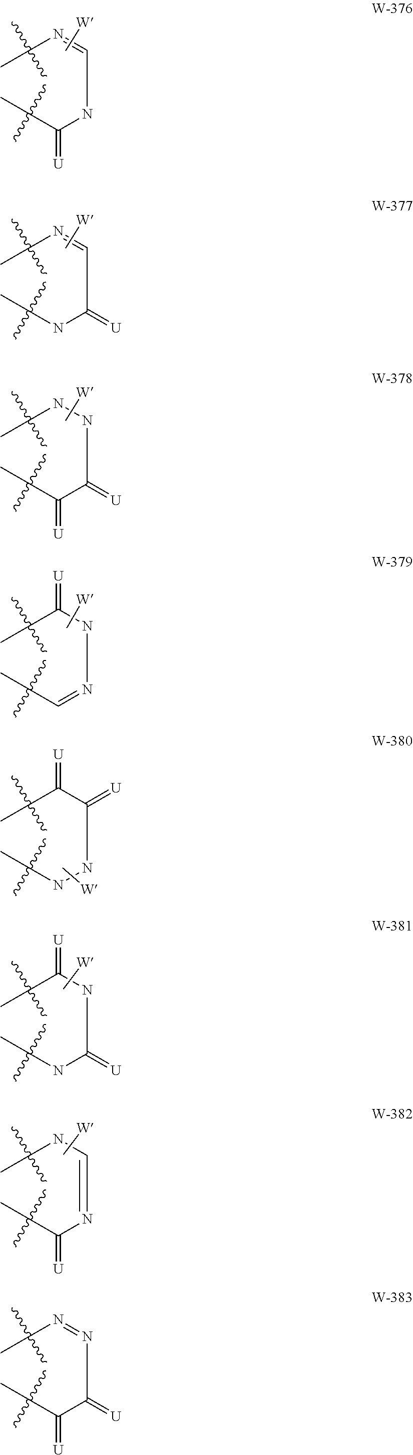

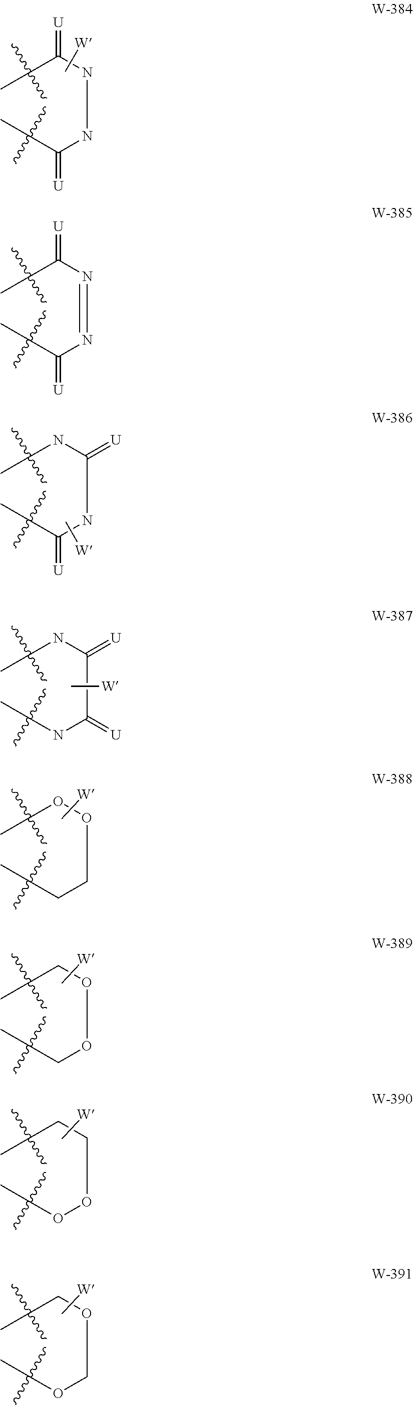

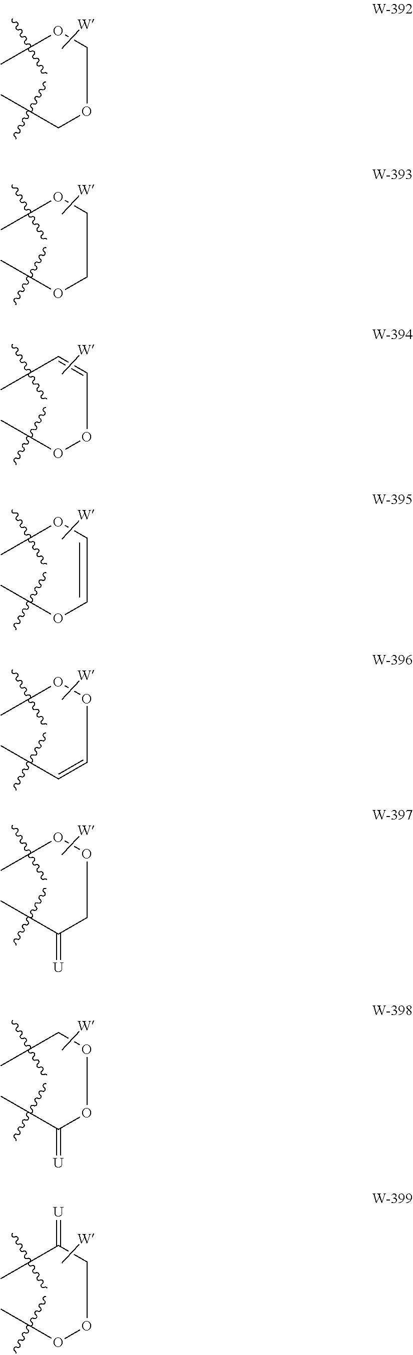

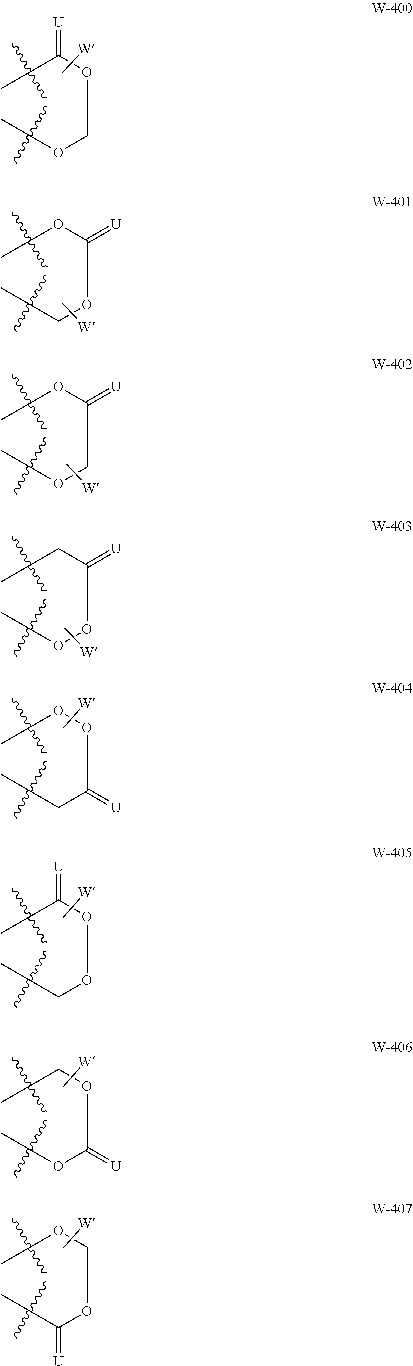

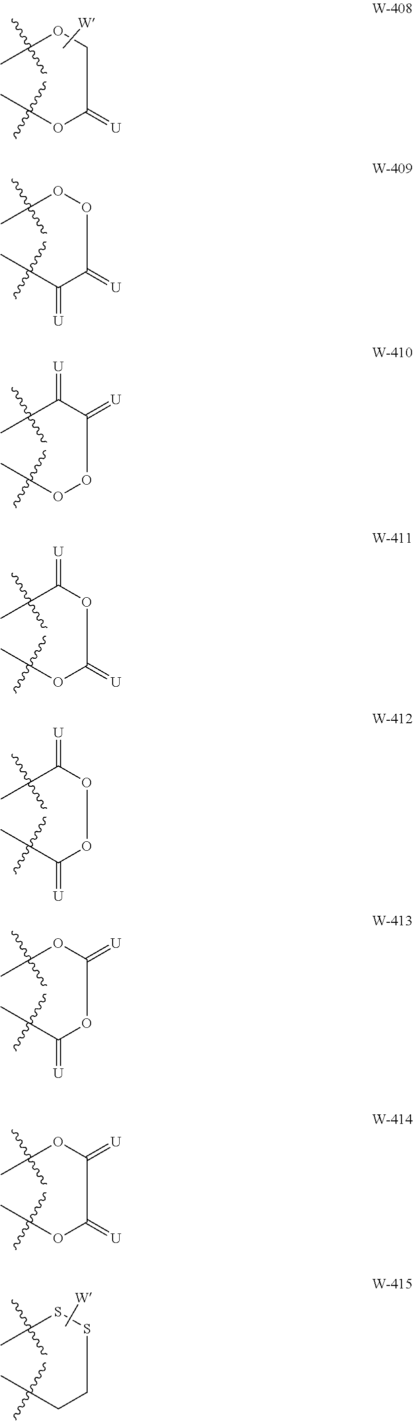

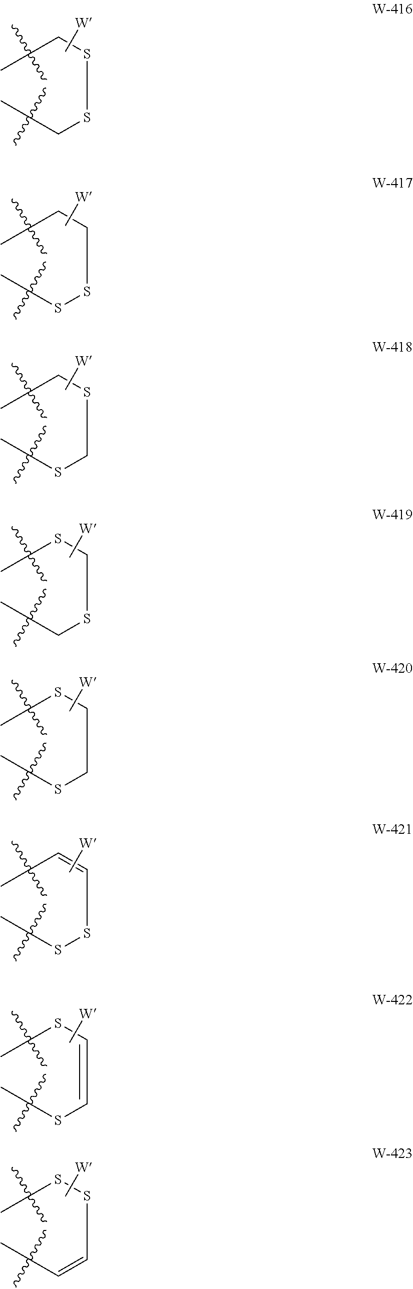

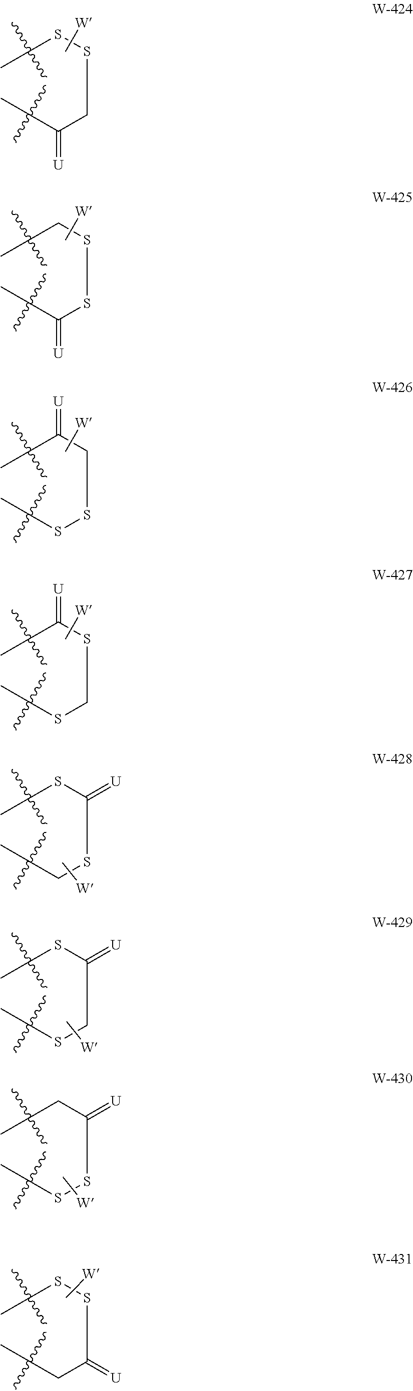

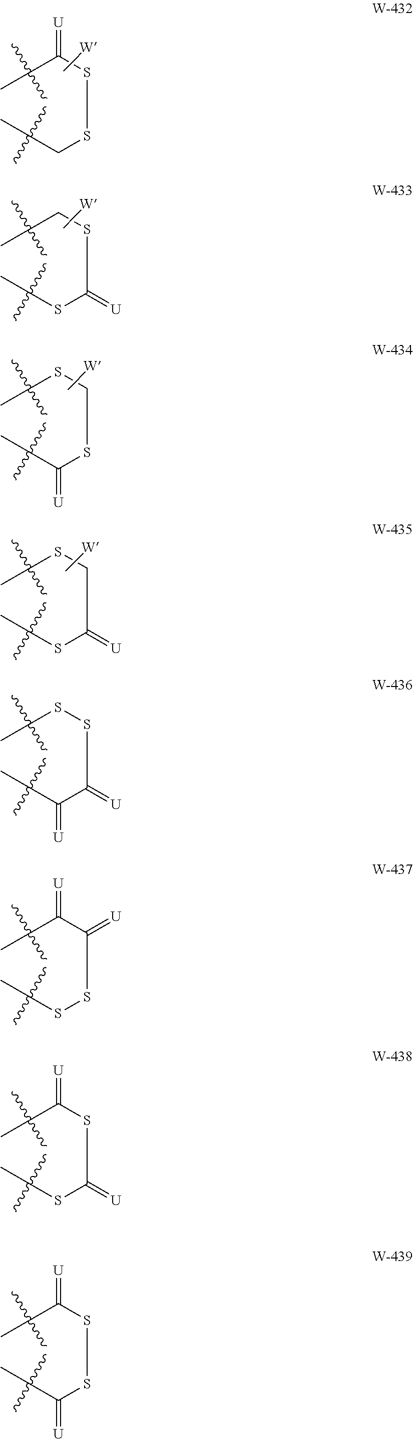

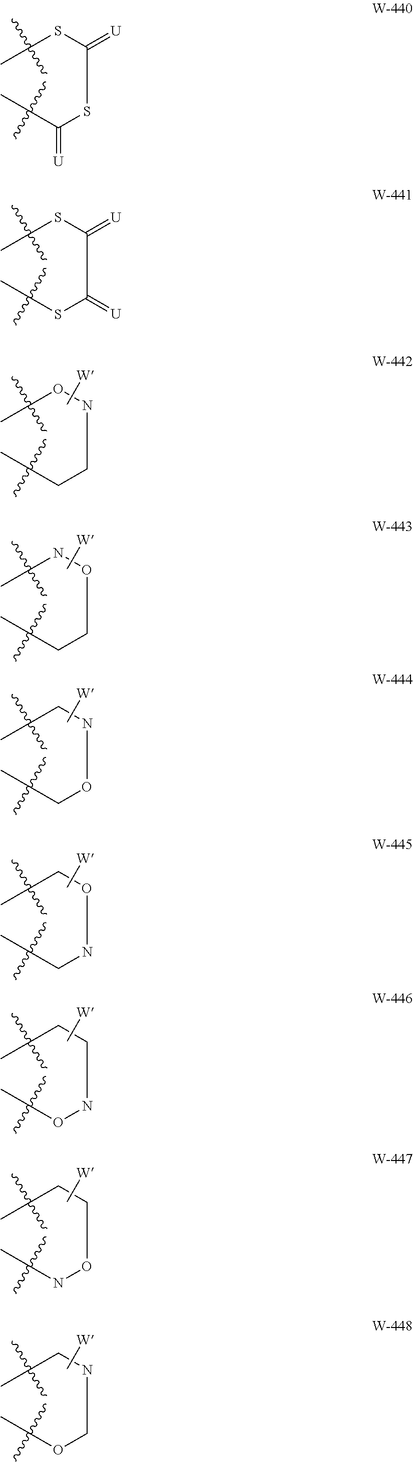

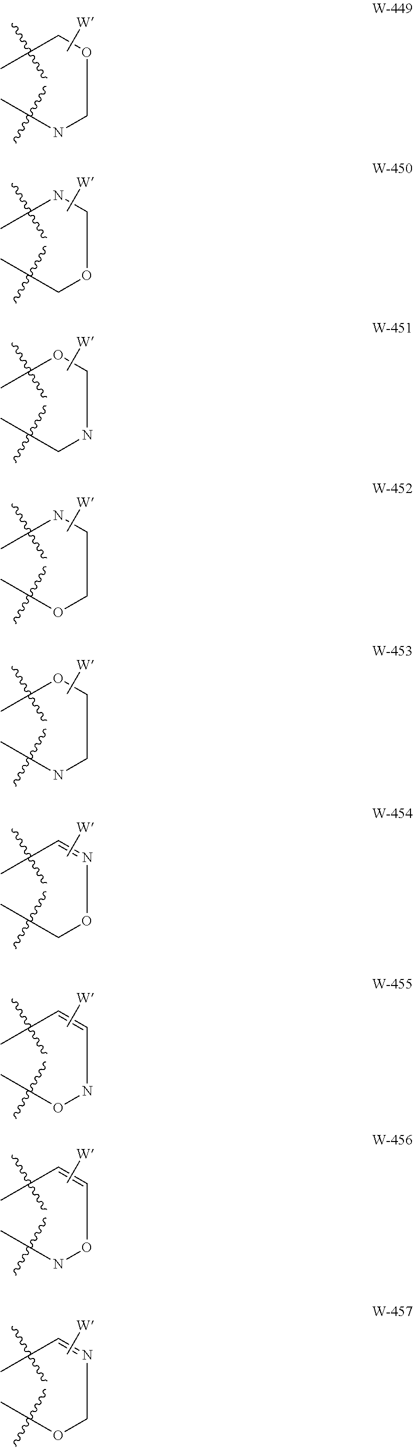

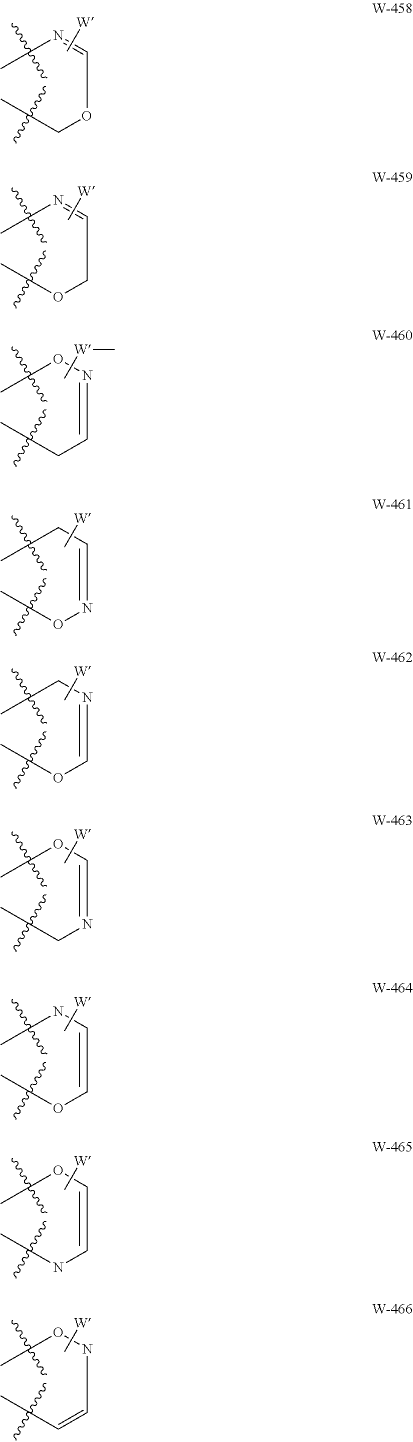

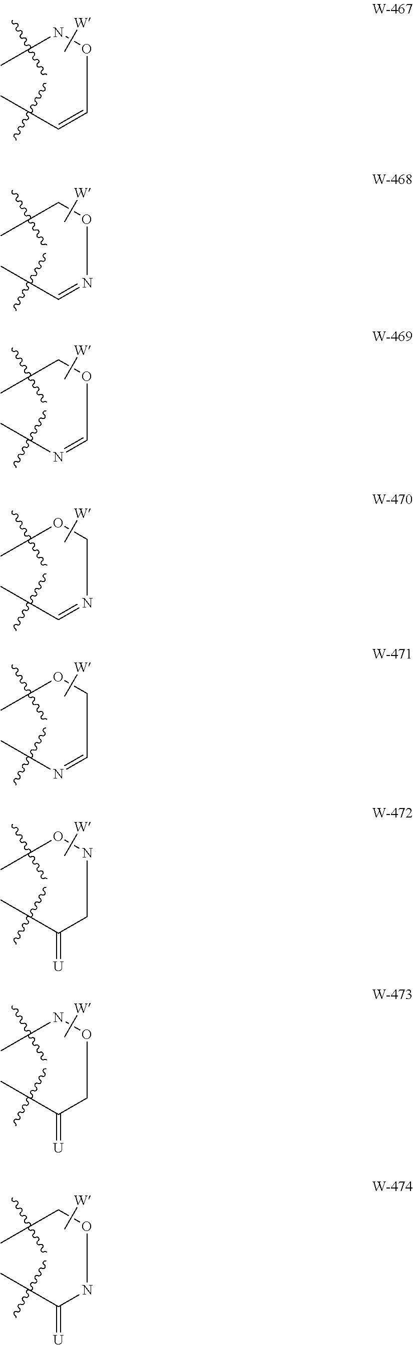

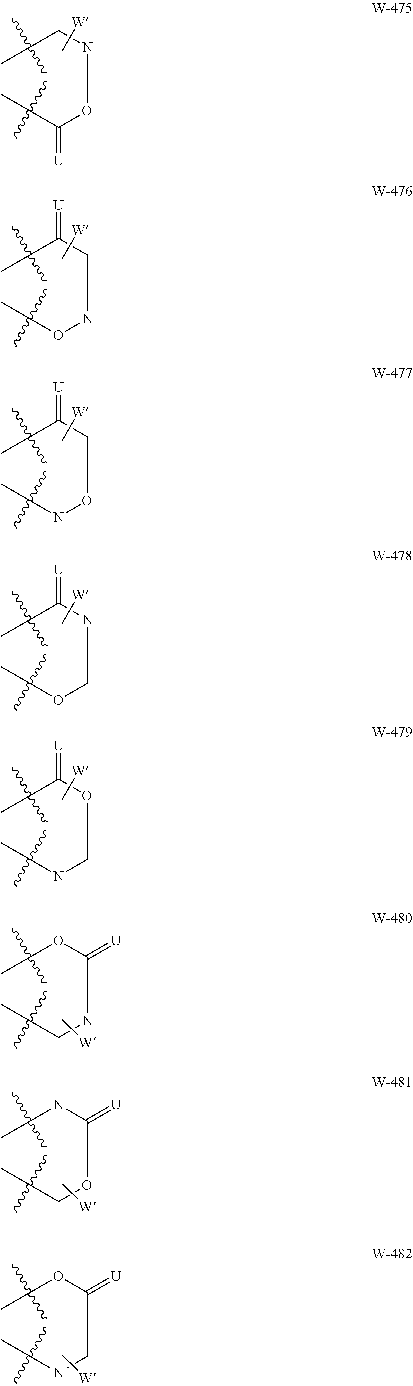

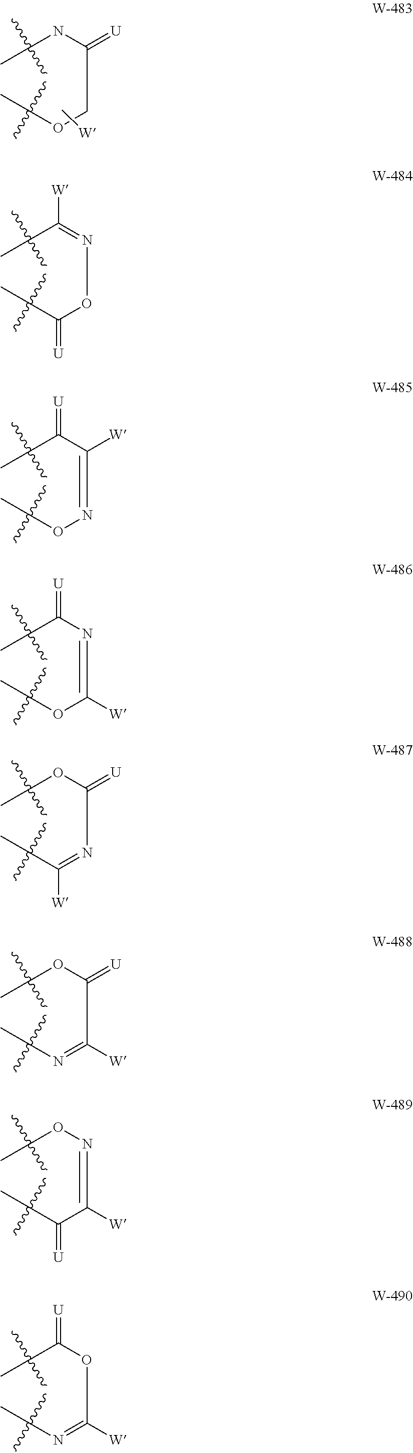

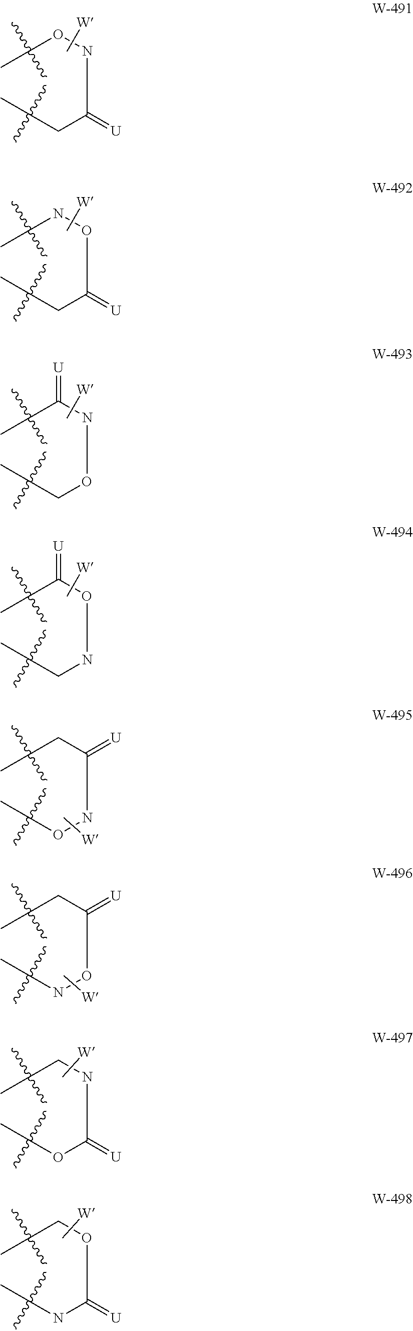

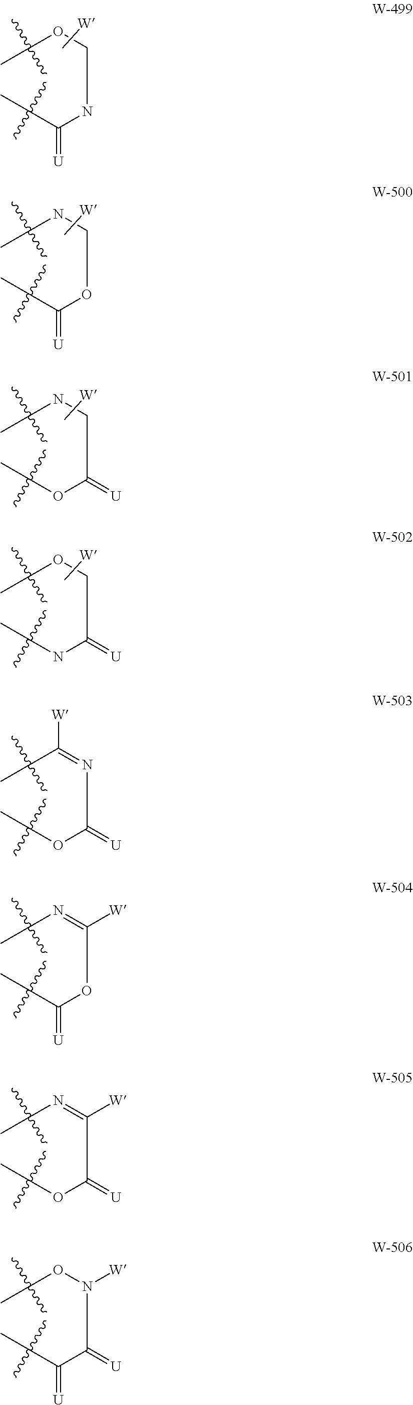

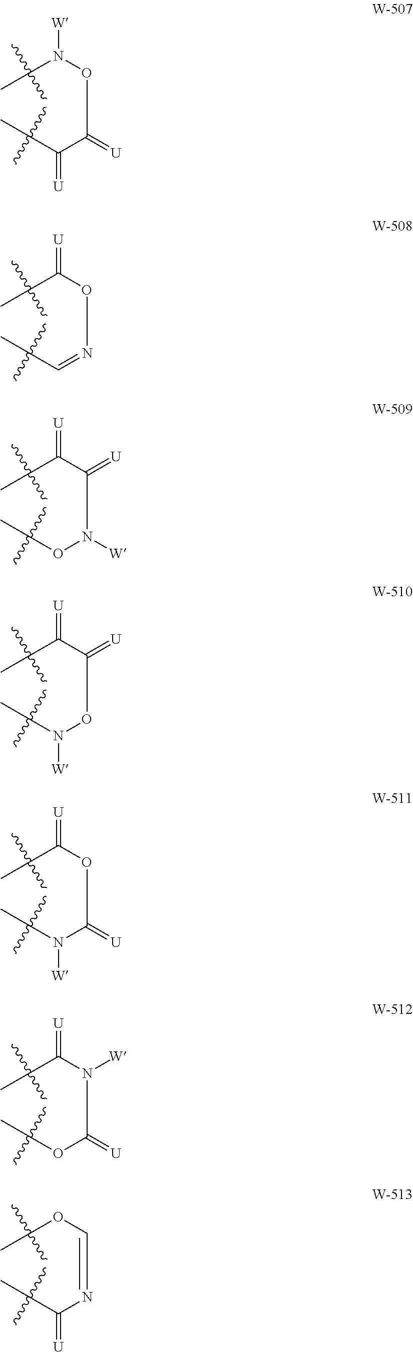

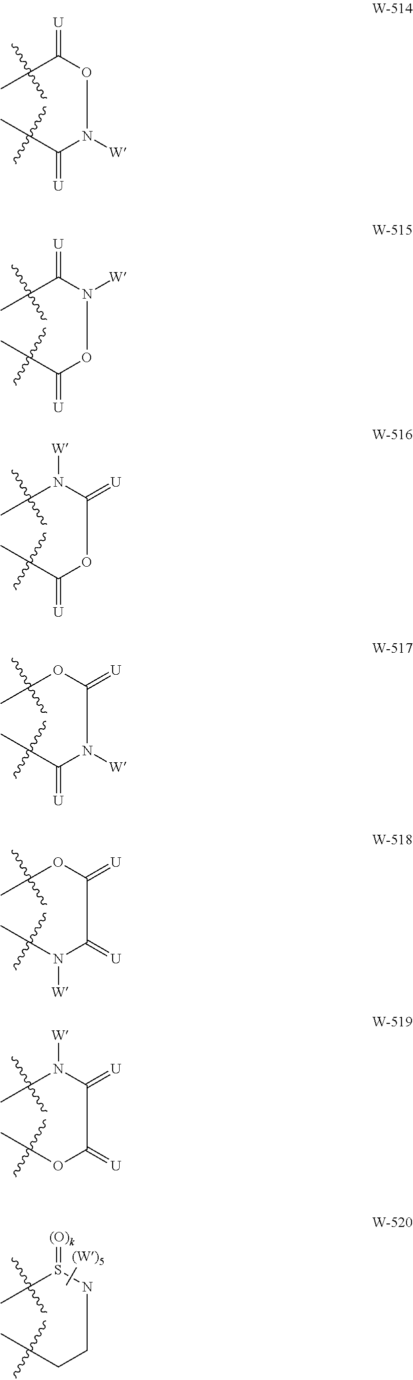

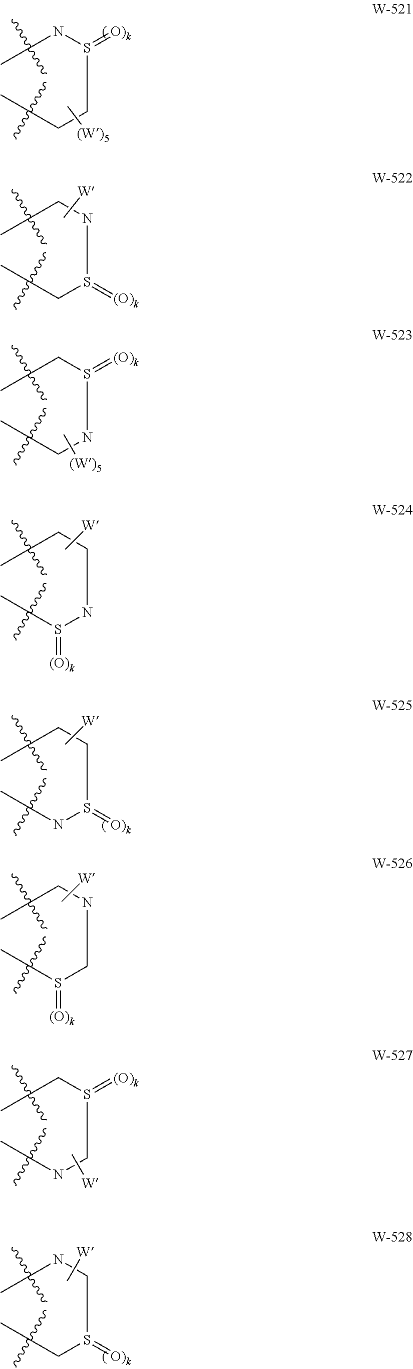

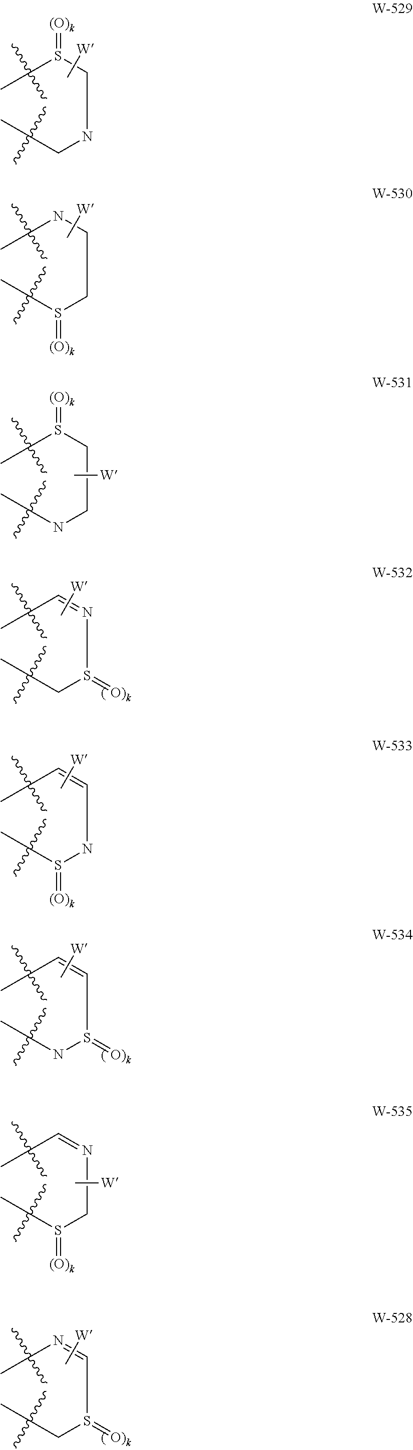

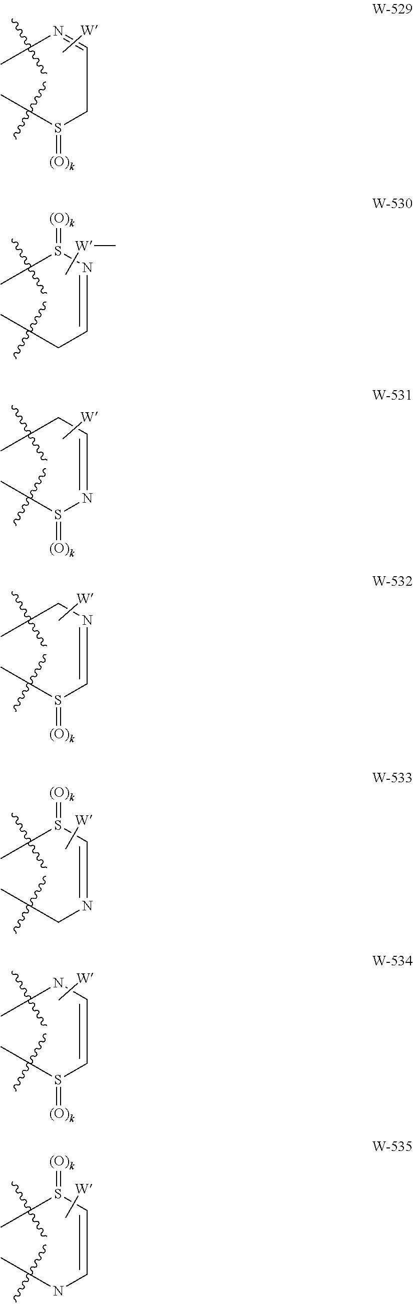

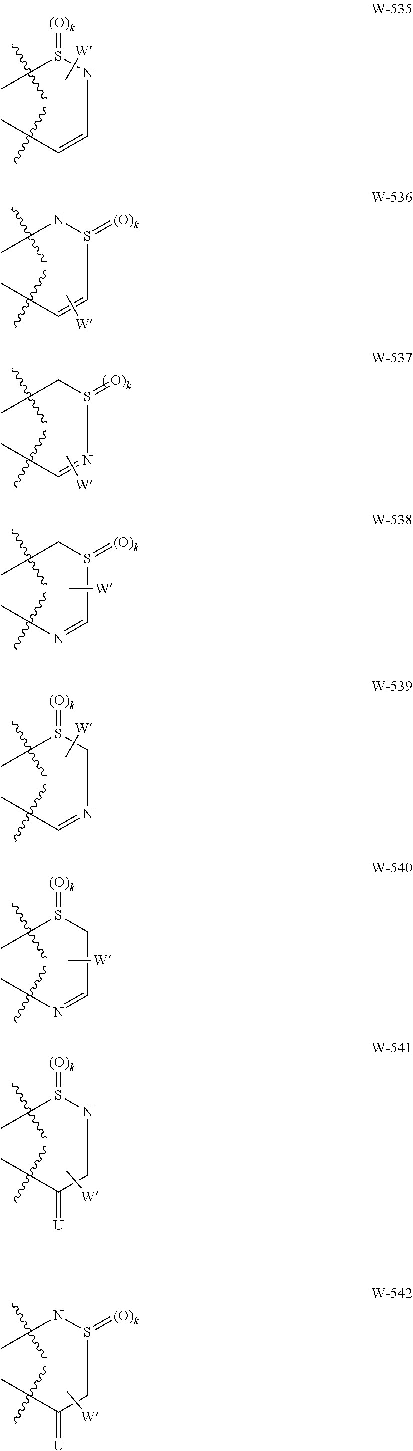

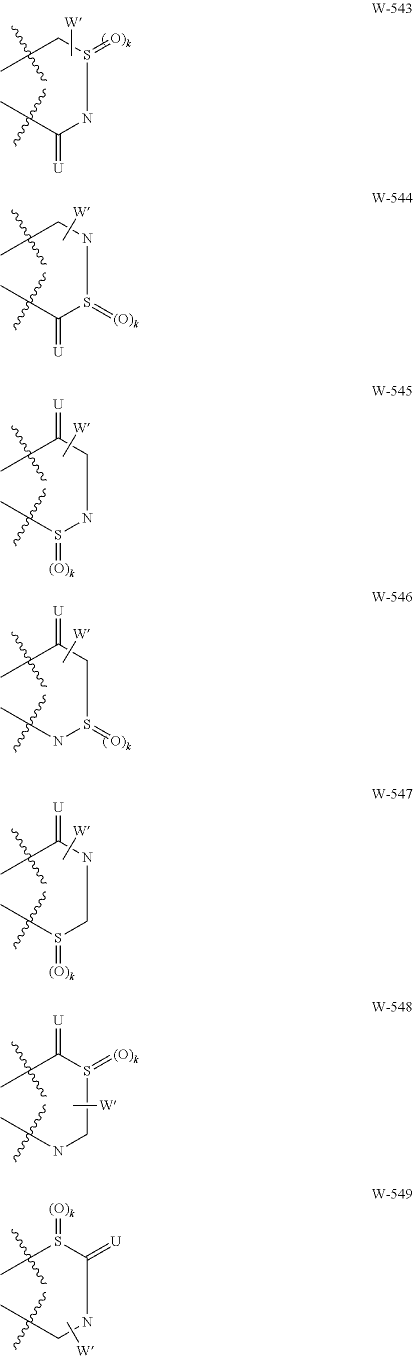

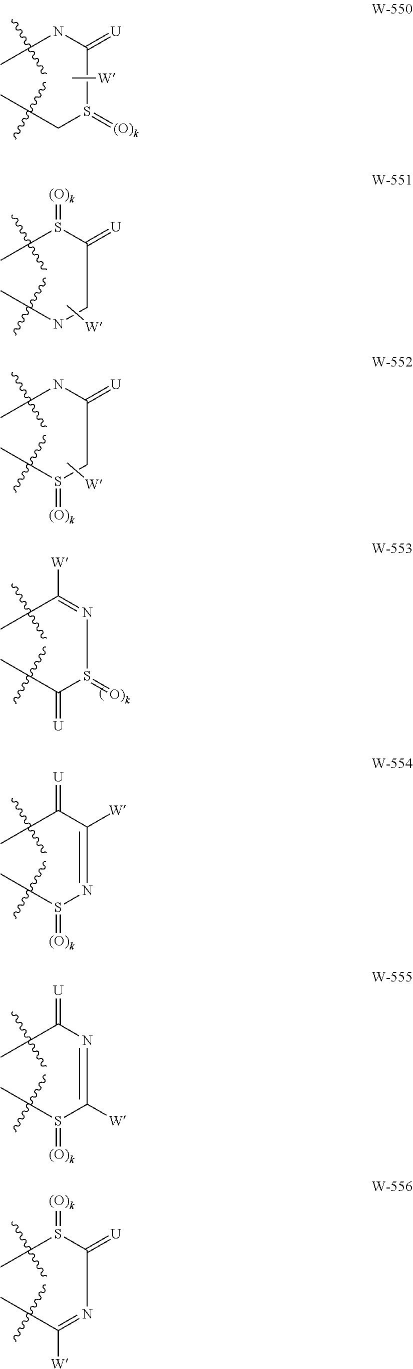

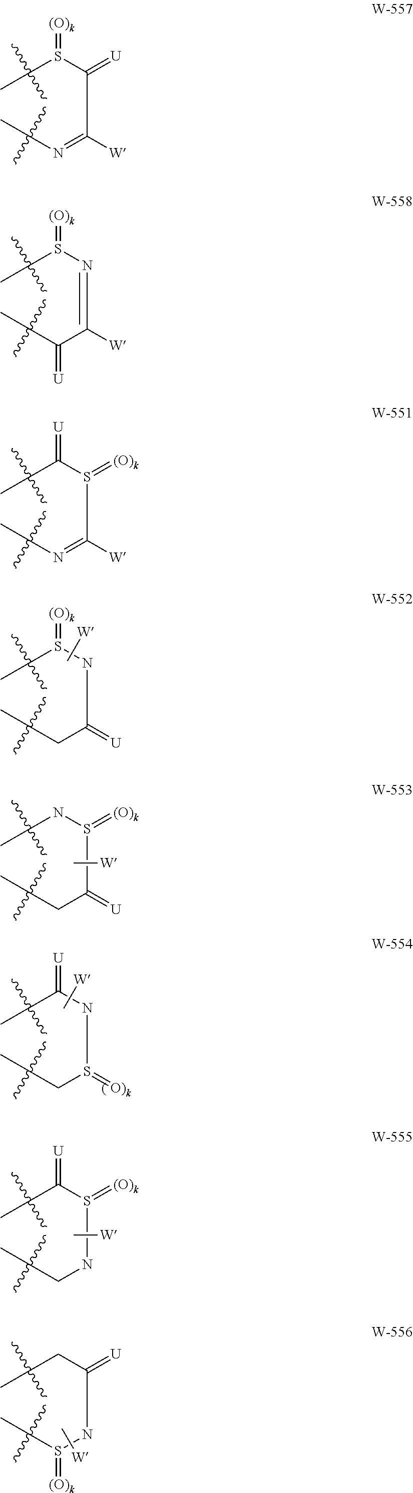

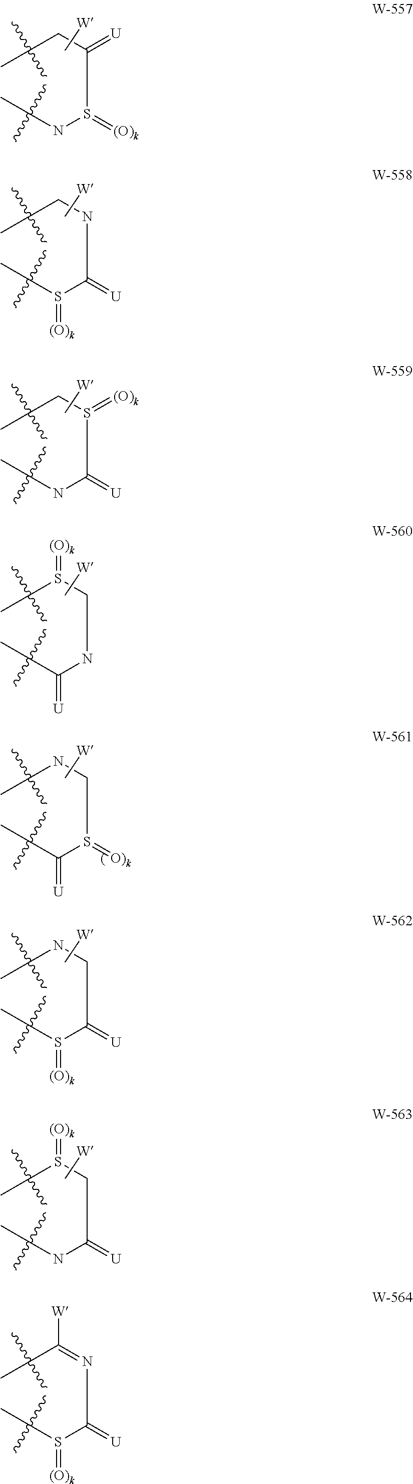

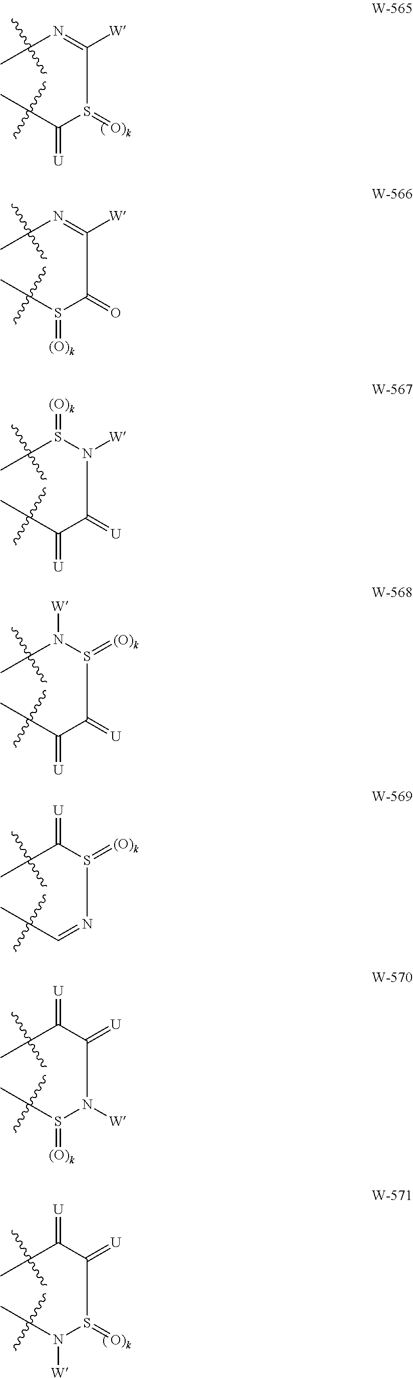

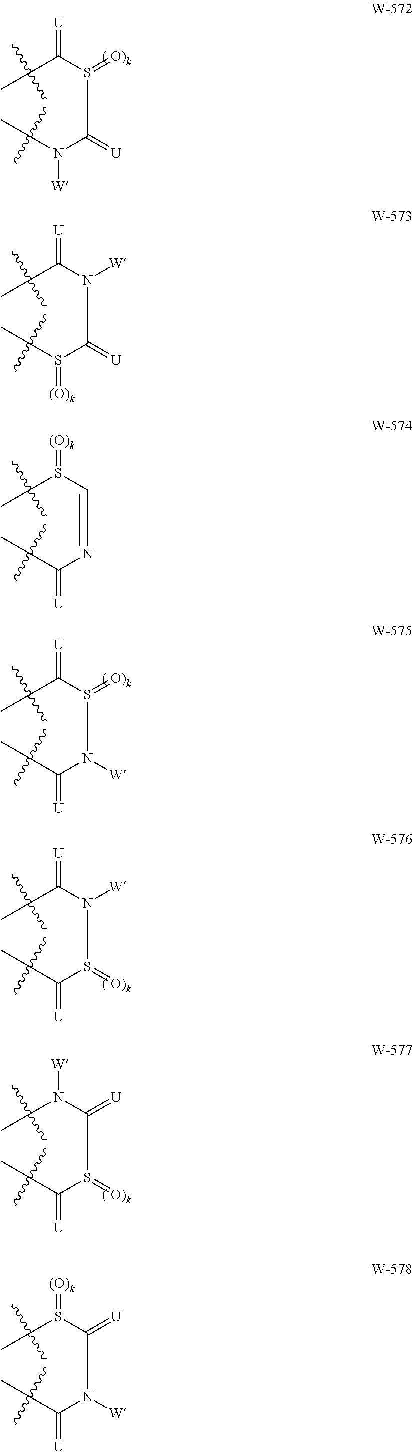

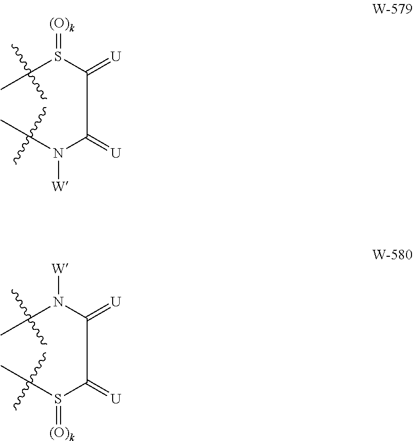

wherein
U represents CH₂, O, S, N—R³ or N—R⁴, preferably represents CH₂, O, S, N—R³, N—R⁴, wherein R³ and R⁴ are selected among hydroxy, C₁₋₆ alkoxy, C₁₋₆haloalkoxy, C₁₋₆haloalkyl, alkyl C₁₋₆ alkyl, C₁₋₆ alkylamino, C₂₋₁₂ dialkylamino, pyridin-2-yl-C₁₋₆alkyl, pyridin-2-yl-C₁₋₆alkoxy, C₁₋₆alkylcarbonyl, C₁₋₆ alkyl-carbonylamino and C₁₋₆haloalkylamino.
k stands for 0, 1 or 2,
W′ represents O⁻, R³, OR³, SR³, NHR³, N(R³)₂, N(R³)N(R³)R³, N(R⁴)N(R³)R³, N(R³)OR³, R⁴, NR⁴, or N₃;
W′ preferably represents O⁻, R³, OR³, SR³, NHR³, N(R³)₂, N(R³)N(R³)R³, N(R⁴)N(R³)R³, N(R³)OR³, R⁴, NR⁴, or N₃ wherein R³ and R⁴ are selected among hydrogen, formyl, C₁₋₆ alkyl, C₂₋₁₂ alkoxyalkyl, C₂₋₁₂ haloalkoxyalkyl, C₂₋₆alkenyl, C₂₋₆alkynyl, C₃₋₈ cycloalkyl, C₄₋₁₂ alkylcycloalkyl, C₄₋₁₂ cycloalkylalkyl, C₁₋₆haloalkyl, C₁₋₆ alklylimino, C₁₋₆haloalkylimino, C₁₋₆alkenyl-carbonyl, C₁₋₆alkynyl-carbonyl, C₁₋₆ haloalkyl-carbonyl, C₁₋₆ hydroxyalkyl-carbonyl, C₁₋₆alkoxy-carbonyl, C₁₋₆haloalkoxy-carbonyl, aminocarbonyl, C₁₋₆alkylamino-carbonyl, C₁₋₆haloalkylamino-carbonyl, C₁₋₆ hydroxyalkylamino-carbonyl, C₂₋₁₂ dialkylamino-carbonyl, C₂₋₆ di(haloalkyl)aminocarbonyl, C₂₋₆alkenylamino-carbonyl, C₂₋₆alkynylamino-carbonyl, C₃₋₆ cycloalkyl-carbonyl, C₄₋₁₂ cycloalkylalkyl-carbonyl, C₃₋₆ cycloalkylamino-carbonyl, C₄₋₁₂ cyclo-alkylalkylamino-carbonyl, C₃₋₆cycloalkyloxy-carbonyl, C₄₋₁₂ cycloalkylalkyloxy-carbonyl, C₁₋₆ haloalkoxy-carbonyl, phenyl-C₁₋₆alkyl, 2-pyridyl-C₁₋₆alkyl, 3-pyridyl-C₁₋₆alkyl, 4-pyridyl-1-C₁₋₆alkyl, phenylcarbonyl, 2-pyridylcarbonyl, 3-pyridylcarbonyl, 4-pyridylcarbonyl, phenyl-1-C₁₋₆alkylcarbonyl, 2-pyridyl-C₁₋₆alkylcarbonyl, 3-pyridyl-C₁₋₆alkylcarbonyl, 4-pyridyl-C₁₋₆alkyl-carbonyl, phenyloxy-carbonyl, 2-pyridyloxy-carbonyl, 3-pyridyloxy-carbonyl, 4-pyridyloxy-carbonyl, phenyl-C₁₋₆ alkyloxy-carbonyl, 2-pyridyl-C₁₋₆alkyloxy-carbonyl, 3-pyridyl-1-C₁₋₆alkyloxy-carbonyl, 4-pyridyl-C₁₋₆ alkyloxy-carbonyl, phenyl-aminocarbonyl, 2-pyridyl-amino-carbonyl, 3-pyridyl-aminocarbonyl, 4-pyridyl-aminocarbonyl, phenyl-C₁₋₆ alkylamino-carbonyl, 2-pyridyl-C₁₋₆alkylamino-carbonyl, 3-pyridyl-C₁₋₆ alkylamino-carbonyl or 4-pyridyl-C₁₋₆alkyl-amino-carbonylcarbonyl, C₁₋₆alkylcarbonyl, C₁₋₆haloalkylcarbonyl, C₁₋₆alkenylcarbonyl, C₁₋₆ haloalkenylcarbonyl, C₁₋₆ alkynylcarbonyl, C₁₋₆haloalkynylcarbonyl, C₁₋₆alkoxycarbonyl, aminocarbonyl, C₁₋₆alkylaminocarbonyl, C₁₋₆haloalkylaminocarbonyl, C₁₋₆ hydroxyalkylamino-carbonyl, C₂₋₁₂ dialkylamino-carbonyl, C₂₋₆ di(haloalkyl)aminocarbonyl, C₂₋₆alkenylaminocarbonyl, C₂₋₆ alkynylaminocarbonyl, C₁₋₆ alkyl-thiocarbonyl, C₃₋₆ cycloalkylcarbonyl, C₄₋₁₂ cycloalkylalkyl-1-carbonyl, C₃₋₆ cycloalkyl-thiocarbonyl, C₄₋₁₂ (total carbon number) cycloalkylalkyl-thiocarbonyl, C₁₋₆ haloalkyl-thiocarbonyl, C₁₋₆ alkylamino-thiocarbonyl, C₃₋₆ cycloalkylamino-carbonyl, C₄₋₁₂ cycloalkylalkylamino-carbonyl, C₃₋₆ cycloalkylamino-thiocarbonyl, C₄₋₁₂ cycloalkylalkyl-aminothiocarbonyl, C₁₋₆ haloalkylamino-thiocarbonyl, C₂₋₁₂ dialkylamino-thiocarbonyl, C₃₋₆ cyclo-alkyloxy-carbonyl, C₄₋₁₂ cycloalkylalkyloxy-carbonyl, C₁₋₆ haloalkoxy-carbonyl, C₁₋₆alkylsulfonyl, C₁₋₆ haloalkylsulfonyl, phenylsulfonyl, phenyl-C₁₋₆alkyl, 2-pyridyl-C₁₋₆alkyl, 3-pyridyl-C₁₋₆alkyl, 4-pyridyl-C₁₋₆alkyl, phenylcarbonyl, 2-pyridylcarbonyl, 3-pyridylcarbonyl, 4-pyridylcarbonyl, phenyl-thiocarbonyl, 2-pyridyl-thiocarbonyl, 3-pyridyl-thiocarbonyl, 4-pyridyl-thiocarbonyl, phenyl-C₁₋₆alkylcarbonyl, 2-pyridyl-C₁₋₆alkylcarbonyl, 3-pyridyl-C_{1-6alkyl} carbonyl, 4-pyridyl-1-C₁₋₆alkylcarbony, phenyl-C₁₋₆ alkyl-thiocarbonyl, 2-pyridyl-C₁₋₆ alkyl-thiocarbonyl, 3-pyridyl-1-C₁₋₆alkyl-thiocarbonyl, 4-pyridyl-C₁₋₆alkyl-thiocarbonyl, phenyl-oxycarbonyl, 2-pyridyloxycarbonyl, 3-pyridyl-oxycarbonyl, 4-pyridyloxycarbonyl, phenyl-C₁₋₆ alkyl-oxy-carbonyl, 2-pyridyl-C₁₋₆alkyloxy-carbonyl, 3-pyridyl-C₁₋₆alkyloxy-carbonyl, 4-pyridyl-1-C₁₋₆alkyloxy-carbonyl, phenyl-aminocarbonyl, 2-pyridyl-aminocarbonyl, 3-pyridyl-aminocarbonyl, 4-pyridyl-aminocarbonyl, phenyl-amino-thiocarbonyl, 2-pyridylamino-thiocarbonyl, 3-pyridyl-amino-thiocarbonyl, 4-pyridylamino-thiocarbonyl, phenyl-C₁₋₆alkylamino-carbonyl, 2-pyridyl-1-C₁₋₆alkylamino-carbonyl, 3-pyridyl-C₁₋₆ alkylamino-carbonyl, 4-pyridyl-C₁₋₆alkylamino-carbonyl, phenyl-C₁₋₆ alkylaminothiocarbonyl, 2-pyridyl-C₁₋₆ alkylamino-thiocarbonyl, 3-pyridyl-C₁₋₆alkyl-amino-thiocarbonyl or 4-pyridyl-C₁₋₆alkylamino-thiocarbonyl, preferably R³ and R⁴ are selected among hydrogen, formyl, C₁₋₆ hydroxyalkyl-carbonyl, C₁₋₆alkoxy-carbonyl, C₁₋₆alkylamino-1-carbonyl, C₁₋₆haloalkylamino-carbonyl, C₂₋₁₂ dialkylamino-carbonyl, C₁₋₆alkylcarbonyl, C₁₋₆haloalkylcarbonyl, C₁₋₆ alkyl-thiocarbonyl, C₁₋₆ alkylamino-thiocarbonyl, C₁₋₆ alkylsulfonyl, pyrimidinyl, and pyridyl.

The present invention does not include the following compounds, which are known from WO 2005/085216, namely

(i) compounds of formula (I), wherein the group (X)ₘ-Q stands for 3,5-dichlorophenyl, n stands for 0, A is oxygen, R¹ stands for CF₃, and the group —W¹—W²—W³—W⁴— stands for the group W-376 with U standing for oxygen and wherein W′ which is bound to the nitrogen atom adjacent to the carbonyl group either represents hydrogen or CH₂-2-pyridyl; and
(ii) compounds of formula (I), wherein the group (X)ₘ-Q stands for 3,5-dichlorophenyl, n stands for 0, A is oxygen, R¹ stands for CF₃, and the group —W¹—W²—W³—W⁴— stands for the group W-23 with U standing for oxygen and wherein W which is bound to the nitrogen atom is either hydrogen, methyl or CH₂-2-pyridyl; and
(iii) compounds of formula (I), wherein the group (X)ₘ-Q stands for 3,5-dichlorophenyl, n stands for 0, A is oxygen, R¹ stands for CF₃, and wherein W¹ is omitted, W² stands for a group C═CH₂, W³ stands for N—CH₂—CF₃ and W⁴ stands for a group C═O.

In another aspect, the invention is directed to compounds of formula (I) wherein
X represents halogen, C₁₋₁₂ alkyl, C₁₋₁₂ haloalkyl, nitro, cyano, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkylthio, C₁₋₁₂ haloalkylsulfinyl, C₁₋₁₂ haloalkylsulfonyl, hydroxy, mercapto, amino, C₁₋₁₂ alkylamino, C₂₋₂₄ (total carbon number) dialkylamino, C₁₋₁₂ (total carbon number) acylamino, C₁₋₁₂ alkoxy-carbonyl-amino, C₁₋₁₂ haloalkoxy-carbonylamino, C₁₋₁₂ alkyl-sulfonylamino group, or C₁₋₁₂ haloalkyl-1-sulfonylamino;
Q represents a group selected among Q-1 to Q-54;
Y represents halogen, C₁₋₁₂ alkyl, C₁₋₁₂ haloalkyl, C₃₋₁₂ cycloalkyl, C₃₋₁₂ cyclo-haloalkyl, nitro, cyano, C₂₋₁₂ alkenyl, C₂₋₁₂ haloalkenyl, C₁₋₁₂ alkoxy, C₁₋₁₂ haloalkoxy, C₁₋₁₂alkylthio, C₁₋₁₂ alkylsulfinyl, C₁₋₁₂alkylsulfonyl, C₁₋₁₂ haloalkylthio, C₁₋₁₂ haloalkylsulfinyl, C₁₋₁₂ haloalkyl-sulfonyl, hydroxy, mercapto, amino, C₁₋₁₂ alkylamino, C₂₋₂₄ (total carbon number) dialkylamino, aminocarbonyl, C₁₋₁₂ alkylaminocarbonyl, C₂₋₂₄ (total carbon number) dialkylaminocarbonyl, C₂₋₁₂ (total carbon number) acylamino, C₁₋₁₂ alkoxy-carbonylamino benzyloxy-carbonylamino C₁₋₁₂haloalkoxy-carbonylamino, C₁₋₁₂alkylsulfonylamino, C₁₋₁₂ haloalkylsulfonylamino or C₃₋₂₆ (total carbon number) trialkylsilyl;
R¹ represents C₁₋₁₂ alkyl, C₃₋₁₂ (total carbon number) cycloalkyl, C₄₋₁₂ (total carbon number) alkylcycloalkyl, C₄₋₁₂ (total carbon number) cycloalkylalkyl, C₂₋₁₂ alkenyl, C₂₋₁₂ alkynyl, C₁₋₁₂ haloalkyl, C₃₋₁₂ (total carbon number) halocycloalkyl, or cyano;
m represents 0, 1, 2, 3, 4 or 5;
n represents 0, 1, 2 or 3;
A represents O, CH₂ or N—R^{x},
R^{x} independently represents hydrogen, cyano, formyl, C₁₋₁₂ alkyl, C₂₋₁₂ alkenyl, C₂₋₁₂ alkynyl, C₃₋₁₂ (total carbon number) cycloalkyl, C₄₋₁₂ (total carbon number) alkylcycloalkyl, C₄₋₁₂ (total carbon number) cycloalkylalkyl, C₁₋₁₂ haloalkyl, phenyl, C₁₋₁₂ alkyl-carbonyl, C₁₋₁₂ alkoxy-carbonyl, C₁₋₁₂ alkyl-aminocarbonyl, C₂₋₂₄ (total carbon number) dialkyl-amino-carbonyl;
W¹, W², W³ and W⁴ independently represents a single bond, CH₂, CH, N, CH—R^{x}, C(R^{x})₂, C═U, O, S, N—R^{y} or N—R^{z} under the prerequisite that a) two or more of W¹, W², W³ and W⁴ are not simultaneously a single bond, b) three or more of W¹, W², W³ and W⁴ are not simultaneously O, S or N—R^{y}, c) three or more of W¹, W², W³ and W⁴ are not simultaneously C=U, d) when two of W¹, W², W³ and W⁴ simultaneously represent O and/or S, at least one carbon atom is present between them, and, e) when at least one of W¹, W², W³ and W⁴ is CH or N, at least one of adjacent W¹, W², W³ and W⁴ is CH or N and forms a double bond.
   U represents CH₂, O, S, N—R^{y} or N—R^{z};
R^{y} independently represents hydrogen, hydroxy, amino, C₁₋₁₂ alkyl, C₂₋₁₂ alkenyl, C₂₋₁₂ alkenyloxy, C₂₋₁₂ alkynyl, C₂₋₁₂ alkynyloxy, C₃₋₁₂ cycloalkyl, C₄₋₁₂ (total carbon number) alkyl-cycloalkyl, C₄₋₁₂ (total carbon number) cycloalkylalkyl, C₁₋₁₂ haloalkyl, C₁₋₁₂ alkylamino, C₂₋₂₄ (total carbon number) dialkyl-amino, C₁₋₁₂ alkoxy, C₁₋₁₂ haloalkoxy, formyl, C₁₋₁₂ alkyl-carbonyl, C₁₋₁₂ haloalkyl-carbonyl, C₁₋₁₂ alkyl-carbonylamino, C₁₋₁₂ haloalkyl-carbonylamino, C₁₋₁₂ alkyl-sulfonyl, C₁₋₁₂ haloalkyl-sulfonyl, phenylsulfonyl, and said substituents other than hydrogen and a formyl group may be substituted with R^{z};
R^{z} is selected among the groups R⁴-1 to R⁴-83 wherein G represents O, S or N, and which may be substituted with at least one group selected among hydrogen, halogen, C₁₋₁₂ alkyl C₁₋₁₂ haloalkyl, C₃₋₁₂ cycloalkyl, C₃₋₁₂ halocycloalkyl, C₁₋₁₂ alkoxy, C₁₋₁₂ haloalkoxy, C₁₋₁₂ alkylthio, C₁₋₁₂ haloalkylthio, C₁₋₁₂ alkylsulfinyl, C₁₋₁₂ haloalkylsulfinyl, C₁₋₁₂ alkylsulfonyl, C₁₋₁₂ haloalkyl-sulfonyl, C₁₋₁₂ alkylamino, C₁₋₁₂ haloalkylamino, cyano, nitro, aminocarbonyl, C₁₋₁₂ alkyl-amino-carbonyl, C₂₋₂₄ (total carbon number) dialkyl-aminocarbonyl, C₁₋₁₂ alkoxy-carbonyl phenyl or pyridyl;

In a further aspect, the invention is also directed to compounds of formula (I) wherein
X independently represents halogen, C₁₋₁₂ alkyl, C₁₋₁₂ haloalkyl, nitro, cyano, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkyl-thio, C₁₋₁₂ haloalkylsulfinyl, C₁₋₁₂ haloalkylsulfonyl, hydroxy, mercapto, amino, C₁₋₁₂ alkylamino, C₂₋₂₄ (total carbon number), dialkylamino, C₁₋₁₂ (total carbon number) acylamino, C₂₋₁₃ (total carbon number) alkoxy-carbonylamino, C₂₋₁₃ (total carbon number) haloalkoxy-carbonylamino, C₁₋₁₂ alkyl-sulfonylamino, a C₁₋₁₂ haloalkylsulfonylamino;
Q is selected among Q-1 to Q-54;
Y independently represents halogen, C₁₋₁₂ alkyl, C₁₋₁₂ haloalkyl, C₃₋₁₂ cycloalkyl, C₃₋₁₂ cyclohaloalkyl, nitro, cyano, C₂₋₁₂ alkenyl, C₂₋₁₂ haloalkenyl, C₁₋₁₂ alkoxy, C₁₋₁₂ haloalkoxy, C₁₋₁₂ alkylthio, C₁₋₁₂ alkylsulfinyl, C₁₋₁₂alkylsulfonyl, C₁₋₁₂ haloalkylthio, C₁₋₁₂ haloalkylsulfinyl, C₁₋₁₂ haloalkylsulfonyl, hydroxy, mercapto, amino, C₁₋₁₂ alkylamino, C₂₋₂₄ (total carbon number) dialkylamino, aminocarbonyl, C₂₋₁₃ (total carbon number) alkylaminocarbonyl, C₃₋₂₅ (total carbon number) dialkylaminocarbonyl, C₁₋₁₂ (total carbon number) acylamino, C₂₋₁₃ (total carbon number) alkoxy-carbonylamino, benzyloxy-carbonylamino, C₂₋₁₃ haloalkoxy-carbonylamino, C₁₋₁₂ alkyl-sulfonylamino, C₁₋₁₂ haloalkylsulfonylamino, a C₃₋₂₆ (total carbon number) trialkylsilyl;
R¹ represents C₁₋₁₂ alkyl, C₃₋₁₂ (total carbon number) cycloalkyl, C₄₋₂₄ (total carbon number) alkylcycloalkyl, C₄₋₂₄ (total carbon number) cycloalkylalkyl, C₂₋₁₂ alkenyl, C₂₋₁₂ alkynyl, C₁₋₁₂ haloalkyl, C₃₋₁₂ (total carbon number) halocycloalkyl, cyano;
m represents 0, 1, 2, 3, 4 or 5,
n represents 0, 1, 2 or 3,
A represents O, CH₂ or N—R^{x};
R^{x} independently represents hydrogen, cyano, formyl, C₁₋₁₂ alkyl, C₂₋₁₂ alkenyl, C₂₋₁₂ alkynyl, C₃₋₁₂ (total carbon number) cycloalkyl, C₄₋₁₂ (total carbon number) alkylcycloalkyl, C₄₋₂₄ (total carbon number) cycloalkylalkyl, C₁₋₁₂ haloalkyl, phenyl, C₂₋₁₃ (total carbon number) alkylcarbonyl, C₂₋₁₃ (total carbon number) alkoxycarbonyl, C₂₋₁₃ (total carbon number) alkylamino-carbonyl or C₃₋₂₅ (total carbon number) dialkylaminocarbonyl,
W¹, W², W³ and W⁴ independently represent a single bond, CH₂, CH, N, O, S, CH—R^{y}, C—R^{y}, C(R^{y})₂, N—R^{z}, CH—R^{xx}, C—R^{xx} or C═U; under the prerequisite that a) two or more of W¹, W², W³ and W⁴ are not simultaneously a single bond, b) three or more of W¹, W², W³ and W⁴ are not simultaneously O, S, N—R^{z} or N—R^{xx}, c) three or more of W¹, W², W³ and W⁴ are not simultaneously C═U, d) when two of W¹, W², W³ and W⁴ simultaneously represent O and/or S, at least one carbon atom is present between them, and, e) when one of W¹, W², W³ and W⁴ is CH, N, C—R^{y} or C—R^{xx}, a double bond is formed within the condensed ring;
R^{Y} independently represents hydroxy, amino, cyano, formyl, halogen, nitro, C₁₋₁₂ alkyl, C₂₋₁₂ alkenyl, C₂₋₁₂ alkynyl, C₃₋₁₂ cycloalkyl, C₄₋₂₄ alkylcycloalkyl, C₄₋₂₄ cycloalkylalkyl, C₁₋₁₂ haloalkyl, C₂₋₁₃ (total carbon number) alkylcarbonyl, C₂₋₁₃ (total carbon number) alkoxycarbonyl, C₂₋₁₃ (total carbon number) alkylaminocarbonyl, C₃₋₁₃ (total carbon number) dialkylaminocarbonyl, C₂₋₁₃ (total carbon number) alkyl-carbonylamino, C₂₋₁₃ (total carbon number) alkylthiocarbonylamino, C₄₋₁₃ (total carbon number) cycloalkyl-carbonylamino, C₅₋₂₅ (total carbon number) cycloalkylalkyl-carbonylamino, C₄₋₁₃ (total carbon number) cycloalkyl-thiocarbonylamino, C₅₋₂₅ (total carbon number) cycloalkylalkylthiocarbonylamino, C₂₋₁₃ (total carbon number) haloalkyl-carbonylamino, C₂₋₁₃ (total carbon number) haloalkylthiocarbonylamino, C₂₋₁₃ (total carbon number) alkylamino-carbonylamino, C₂₋₁₃ (total carbon number) alkylamino-thiocarbonylamino, C₄₋₁₃ (total carbon number) cycloalkylamino-carbonylamino, C₅₋₂₅ (total carbon number) cycloalkylalkylamino-carbonylamino, C₄₋₁₃ (total carbon number) cycloalkylamino-thiocarbonylamino, C₅₋₂₅ (total carbon number) cycloalkylalkylaminothiocarbonylamino, C₂₋₁₃ (total carbon number) haloalkylamino-carbonylamino, C₂₋₁₃ (total carbon number) haloalkylamino-thiocarbonylamino, C₃₋₂₅ (total carbon number) dialkylamino-carbonylamino, C₂₋₂₅ (total carbon number) dialkylaminothiocarbonylamino, C₂₋₁₃ (total carbon number) alkylaminocarbonyloxy, C₂₋₁₃ (total carbon number) alkylaminocarbonylthio, C₄₋₁₃ (total carbon number) cycloalkylamino-carbonyloxy, C₅₋₂₅ (total carbon number) cycloalkylalkylaminocarbonyloxy, C₄₋₁₃ (total carbon number) cycloalkylaminocarbonylthio, C₅₋₂₅ (total carbon number) cycloalkylalkylaminocarbonyl-thio, C₂₋₁₃ (total carbon number) haloalkylaminocarbonyloxy, C₂₋₁₃ (total carbon number) haloalkyl-aminocarbonylthio, C₃₋₂₅ (total carbon number) dialkylaminocarbonyloxy, C₃₋₂₅ (total carbon number) dialkylaminocarbonylthio, C₂₋₁₃ (total carbon number) alkoxy-carbonylamino, C₄₋₁₃ (total carbon number) cycloalkyloxy-carbonylamino, C₅₋₂₅ (total carbon number) cycloalkylalkyl-oxy-carbonylamino, C₂₋₁₃ (total carbon number) haloalkoxy-carbonylamino, C₁₋₁₂ alkyl-sulfonylamino, C₁₋₁₂haloalkylsulfonylamino, and said substituents other than cyano, formyl, halogen and nitro may be substituted with R^{xx};
U represents CH₂, O, S, N—R^{z} or N—R^{xx};
R^{z} represents hydrogen, hydroxy, amino, C₁₋₁₂ alkyl, C₁₋₁₂ haloalkyl, C₂₋₁₂ alkenyl, C₂₋₁₂ alkenyloxy, C₂₋₁₂ alkynyl, C₂₋₁₂ alkynyloxy, C₃₋₁₂ cycloalkyl, C₄₋₂₄ alkylcycloalkyl, C₄₋₂₄ cyclo-alkylalkyl, C₁₋₁₂ alkylamino, C₂₋₂₄ (total carbon number) dialkylamino, C₁₋₁₂ alkoxy, C₁₋₁₂ haloalkoxy, formyl group, a C₂₋₁₃ (total carbon number) alkylcarbonyl, C₂₋₁₃ (total carbon number) haloalkylcarbonyl, C₂₋₁₃ (total carbon number) alkoxycarbonyl, C₂₋₁₃ (total carbon number) alkylaminocarbonyl, C₂₋₁₃ (total carbon number) alkyl-carbonylamino, C₂₋₁₃ (total carbon number) haloalkyl-carbonylamino, C₂₋₁₃ (total carbon number) alkoxy-carbonylamino, C₂₋₁₃ (total carbon number) alkylamino-carbonylamino, C₁₋₁₂ (total carbon number) alkylsulfonyl, C₁₋₁₂ (total carbon number) haloalkylsulfonyl, phenylsulfonyl, C₂₋₁₃ (total carbon number) alkylaminocarbonyl group which may be substituted or a C₂₋₁₃ (total carbon number) haloalkylaminocarbonyl, and said substituents other than hydrogen and formyl may be substituted with R^{xx};
R^{xx} is selected among the groups R⁴-1 to R⁴-83 wherein G represents O, S or N, and which may be substituted with at least one group selected among hydrogen, halogen, C₁₋₁₂ alkyl, C₁₋₁₂ haloalkyl, C₃₋₁₂ cycloalkyl, C₃₋₁₂ halocycloalkyl, C₁₋₁₂ alkoxy, C₁₋₁₂ haloalkoxy, C₁₋₁₂ alkylthio, C₁₋₁₂ haloalkylthio, C₁₋₁₂ alkylsulfinyl, C₁₋₁₂ haloalkylsulfinyl, C₁₋₁₂ alkylsulfonyl, C₁₋₁₂ haloalkyl-sulfonyl, C₁₋₁₂ alkylamino, C₁₋₁₂ haloalkylamino, cyano, nitro, aminocarbonyl, C₁₋₁₂ alkyl-amino-carbonyl, C₂₋₂₄ (total carbon number) dialkyl-aminocarbonyl, C₁₋₁₂ alkoxy-carbonyl phenyl or pyridyl.

The compounds according to the invention have asymmetric carbons, and thus the compounds also include optically active species. Moreover, the present invention also includes N-oxides and salts of the compounds according to the invention.

The present application is further directed to the following embodiments:

Embodiment A: Compounds having the structure (I-a-1) or (I-a-2), wherein the chemical groups A, R¹, W¹, W², W³ and W⁴ are as defined herein, and wherein each X₁, X₂ and X₃ are as defined herein for X, and wherein each Y₁, Y₂ and Y₃ are as defined herein for Y.

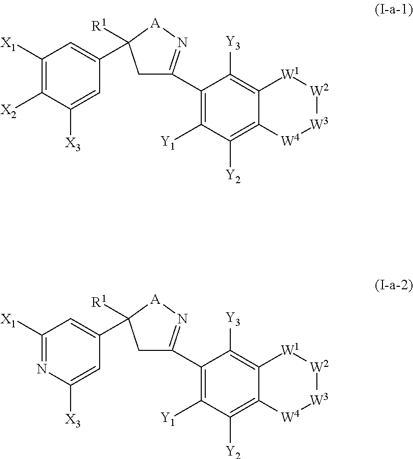

Embodiment B: Compounds as defined in embodiment A, wherein the group —W¹—W²—W³—W⁴—is selected among W-5, W-8, W-12, W-16, W-17, W-18, W-20, W-22, W-23, W-24, W-30, W-31, W-33, W-38, W-39, W-40, W-41, W-42, W-44, W-45, W-46, W-53, W-54, W-64, W-76, W-79, W-86, W-98, W-99, W-114, W-115, W-134, W-157, W-161, W-173, W-223, W-224, W-225, W-241, W-315, W-337, W-339, W-344, W-345, W-348, W-351 and W-357, preferably W-5, W-7, W-11, W-16, W-17, W-20, W-39, W-44, W-45, W-134, W-138, W-158, W-161, W-222, W-225, W-315, W-337, W-340, W-351 and W-357, more preferably W-5 and W-134, or W-16 and W-39.

Embodiment C: Compounds as defined in embodiment A or B, wherein A represents oxygen or CH₂, R¹ represents CF₃, and/or X₁, X₂, X₃ are independently selected among hydrogen, halogen, amino, C₁₋₆ alkylamino, C₂₋₁₂ dialkylamino, nitro, C₁₋₆alkoxy, and C₁₋₆haloalkyl and Y₁, Y₂, Y₃ is H.

Embodiment D: Compounds having the structure (I-d-1), wherein the chemical groups A, R¹, Q, X, Y, m, n and W′ are as defined herein and wherein W″ is selected among hydrogen, halogen, hydroxy, thiol, cyano, C₁₋₆ alkyl, C₁₋₆haloalkyl, C₁₋₆ alkoxy, C₁₋₆alkylsulfenyl, C₁₋₆ alkylsulfinyl, C₁₋₆ alkylsulfonyl.

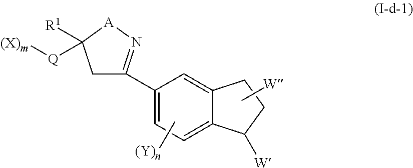

Embodiment D1: Compounds as defined in embodiment D, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆haloalkyl.

Embodiment D2: Compounds as defined in embodiment D or D1, wherein A represents oxygen or CH₂, R¹ represents CF₃, and Y is H.

In these embodiments, W′ preferably represents amino, hydroxy, carbonylamino C₁₋₆ alkyl-carbonylamino, C₁₋₆alkylcarbonyl-C₁₋₆alkyl-carbonylamino, C₁₋₆ alkylsulfenyl-C₁₋₆ alkyl-carbonylamino, C₁₋₆alkylsulfenyl-C₁₋₆alkyl-carbonylamino, C₁₋₆ alkylsulfinyl-C₁₋₆alkyl-carbonyl-amino, C₁₋₆ alkylsulfonyl-C₁₋₆alkyl-carbonylamino, C₁₋₆haloalkyl-carbonylamino, cyano-C₁₋₆ alkyl-carbonylamino C₁₋₆ alkenyl-carbonylamino, C₁₋₆haloalkenyl-carbonylamino, C₁₋₆ alkynyl-carbonylamino, C₁₋₆haloalkynyl-carbonylamino, C₁₋₆ alkoxy-carbonylamino, C₁₋₆haloalkoxy-carbonylamino, cyano-C₁₋₆ alkoxy-carbonylamino, C₁₋₆alkoxy-C₁₋₆alkylamino-carbonylamino, C₁₋₆ alkoxy-C₁₋₆ alkoxy-carbonylamino, amino-carbonylamino, C₁₋₆ alkylamino-carbonylamino, C₁₋₆haloalkylamino-carbonylamino, hydroxyC₁₋₆alkylamino-carbonylamino, cyano-C₁₋₆ alkyl-amino-carbonylamino, C₂₋₁₂ dialkylamino-carbonylamino, C₂₋₁₂ dialkylamino-C₁₋₆ alkyl-carbonyl-amino, C₂₋₆ di(haloalkyl)amino-carbonylamino, C₂₋₆ alkenylamino-carbonylamino, C₂₋₆ alkynyl-amino-carbonylamino, C₁₋₆ alkoxy-amino-carbonylamino, C₁₋₆ alkylsulfonylamino, C₁₋₆ alkenyl-oxy-amino-carbonylamino, C₁₋₆alkyl-thiocarbonylamino, C₃₋₆ cycloalkyl-carbonylamino, C₃₋₆ halocycloalkyl-carbonylamino, C₄₋₁₂ cycloalkylalkyl-carbonylamino, C₃₋₆ cycloalkyl-thiocarbonylamino, C₄₋₁₂ cycloalkylalkyl-thiocarbonylamino, C₁₋₆haloalkyl-thiocarbonylamino, C₁₋₆ alkylamino-thiocarbonylamino, C₃₋₆ cycloalkylamino-carbonylamino, C₃₋₆ halocycloalkyl-amino-carbonylamino, C₄₋₁₂ cycloalkylalkylamino-carbonylamino, C₃₋₆ cycloalkylamino-thio-carbonylamino, C₄₋₁₂ cycloalkylalkylaminothiocarbonylamino, C₁₋₆ haloalkylamino-thiocarbonyl-amino, C₂₋₁₂ dialkylamino-thiocarbonylamino, C₃₋₆ cycloalkyloxy-carbonylamino, C₄₋₁₂ cycloalkyl-alkyloxy-carbonylamino, C₁₋₆ haloalkoxy-carbonylamino, C₁₋₆ alkylsulfonylamino, C₁₋₆haloalkyl-sulfonylamino, C₁₋₆alkylsulfonylamino, phenylsulfonylamino, phenyl-C₁₋₆alkylamino, 2-pyridyl-C₁₋₆ alkylamino, 3-pyridyl-C₁₋₆ alkylamino, 4-pyridyl-C₁₋₆ alkylamino, phenyl-carbonylamino, halo-phenyl-carbonylamino, azetidine-1-yl-carbonylamino, pyrrolidine-1-yl-carbonylamino, N-methyl-pyrrolidine-2-yl-carbonylamino, furan-2-yl-carbonylamino, trifluoromethylphenyl-carbonylamino, phenylamino-carbonylamino, halophenylamino-carbonylamino, trifluoromethyl-phenylamino-carbonylamino, 2-pyridyl-carbonylamino, 3-pyridyl-carbonylamino, 4-pyridyl-carbonylamino, phenyl-thiocarbonylamino, 2-pyridyl-thiocarbonylamino, 3-pyridyl-thiocarbonyl-amino, 4-pyridyl-thiocarbonylamino, phenyl-C₁₋₆ alkyl-carbonylamino, 2-pyridyl-C₁₋₆ alkyl-carbonylamino, 3-pyridyl-C₁₋₆alkylcarbonyl, 4-pyridyl-C₁₋₆alkylcarbony, phenyl-C₁₋₆ alkyl-thiocarbonyl, 2-pyridyl-C₁₋₆ alkyl-thiocarbonyl, 3-pyridyl-C₁₋₆alkyl-thiocarbonylamino, 4-pyridyl-C₁₋₆ alkyl-thiocarbonylamino, phenyl-oxy-carbonylamino, 2-pyridyloxy-carbonylamino, 3-pyridyloxy-carbonylamino, 4-pyridyloxy-carbonylamino, phenyl-C₁₋₆ alkyloxy-carbonylamino, 2-pyridyl-C₁₋₆alkyloxy-carbonylamino, 3-pyridyl-C₁₋₆ alkyloxy-carbonylamino, 4-pyridyl-C₁₋₆ alkyloxy-carbonylamino, phenyl-amino-carbonylamino, 2-pyridyl-amino-carbonylamino, 3-pyridyl-amino-carbonylamino, 4-pyridyl-amino-carbonylamino, phenyl-amino-thiocarbonyl-amino, 2-pyridylamino-thiocarbonylamino, 3-pyridylamino-thiocarbonylamino, 4-pyridylamino-thiocarbonylamino, phenyl-C₁₋₆ alkylamino-carbonylamino, 2-pyridyl-C₁₋₆alkylamino-carbonyl-amino, 3-pydidyl-C₁₋₆alkylamino-carbonylamino, 4-pyridyl-C₁₋₆alkylamino-carbonylamino, phenyl-C₁₋₆ alkylamino-thiocarbonylamino, 2-pyridyl-C₁₋₆alkylamino-thiocarbonylamino, 3-pydidyl-C₁₋₆alkylamino-thiocarbonylamino or 4-pyridyl-C₁₋₆alkylamino-thiocarbonylamino, 1,2,3-triazole-4-trimethylsilyl-1-yl, 1,2,3-triazole-1-yl, succinimide-1-yl, —N₃, phthalimide-2-yl, C₁₋₆ alkylcarbonyloxy, more preferably represents amino, hydroxy, carbonylamino, C₁₋₆ alkyl-sulfonylamino, C₁₋₆ alkylamino-carbonylamino, C₁₋₆ alkylthiocarbonylamino, C₁₋₆ alkyl-carbonyl-amino, C₁₋₆ alkylcarbonyl-C₁₋₆ alkyl-carbonylamino, C₁₋₆ alkylsulfenyl-C₁₋₆ alkyl-carbonylamino, C₁₋₆ alkylsulfenyl-C₁₋₆ alkyl-carbonylamino, C₁₋₆ alkylsulfinyl-C₁₋₆ alkyl-carbonylamino, C₁₋₆ alkyl-sulfonyl-C₁₋₆ alkyl-carbonylamino, C₁₋₆haloalkyl-carbonylamino, cyano-C₁₋₆alkyl-carbonylamino C₁₋₆ alkenyl-carbonylamino, C₁₋₆ alkynyl-carbonylamino, C₁₋₆ alkoxy-carbonylamino, C₁₋₆ haloalkoxy-carbonylamino, cyano-C₁₋₆alkoxy-carbonylamino, C₁₋₆ alkoxy-C₁₋₆ alkylamino-carbonylamino, C₁₋₆ alkoxy-C₁₋₆ alkoxy-carbonylamino, amino-carbonylamino, C₁₋₆ alkylamino-carbonylamino, C₁₋₆ haloalkylamino-carbonylamino, hydroxyC₁₋₆alkylamino-carbonylamino, cyano-C₁₋₆ alkylamino-carbonylamino, C₂₋₁₂ dialkylamino-carbonylamino, C₂₋₁₂ dialkylamino-C₁₋₆ alkyl-carbonylamino, C₂₋₆ alkynylamino-carbonylamino, C₂₋₆ alkenylamino-carbonylamino, C₁₋₆ alkoxy-amino-carbonylamino, C₁₋₆ alkenyloxy-amino-carbonylamino, C₃₋₆ cycloalkyl-carbonyl-amino, C₃₋₆halocycloalkyl-carbonylamino, C₄₋₁₂ cycloalkylalkyl-carbonylamino, C₁₋₆ alkylamino-thiocarbonylamino, C₃₋₆ cycloalkylamino-carbonylamino, C₃₋₆halocycloalkylamino-carbonyl-amino, C₄₋₁₂ cycloalkylalkylamino-carbonylamino, C₁₋₆ haloalkylsulfonylamino, phenyl-carbonyl-amino, halo-phenyl-carbonylamino, azetidine-1-yl-carbonylamino, pyrrolidine-1-yl-carbonylamino, N-methyl-pyrrolidine-2-yl-carbonylamino, furan-2-yl-carbonylamino, trifluoromethylphenyl-carbonylamino, phenylamino-carbonylamino, halophenylamino-carbonylamino, trifluoromethyl-phenylamino-carbonylamino, 1,2,3-triazole-4-trimethylsilyl-1-yl, 1,2,3-triazole-1-yl, succinimide-1-yl, —N₃, phthalimide-2-yl, C₁₋₆ alkylcarbonyloxy, most preferably represents C₁₋₆ haloalkyl-carbonylamino, C₁₋₆ alkylamino-carbonylamino, C₁₋₆ haloalkylamino-carbonylamino, C₁₋₆ alkylamino-carbonylamino, C₁₋₆ alkyl-carbonylamino, and C₁₋₆ haloalkylamino-carbonylamino.

Embodiment E: Compounds having the structure (I-e-1), wherein the chemical groups A, R¹, Q, X, Y, m, n, and W′ are as defined herein and wherein W″ is selected among hydrogen, halogen, hydroxy, thiol, cyano, C₁₋₆ alkyl, C₁₋₆haloalkyl, C₁₋₆ alkoxy, C₁₋₆alkylsulfenyl, C₁₋₆ alkylsulfinyl, C₁₋₆alkylsulfonyl.

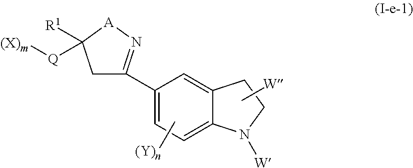

Embodiment E1: Compounds as defined in embodiment E, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment E2: Compounds as defined in embodiment E or E1, wherein A represents oxygen or CH₂, R¹ represents CF₃, and Y is H.

In these embodiments, W′ preferably represents hydrogen, C₁₋₆ alky, C₁₋₆haloalky, C₁₋₆ alkyl-carbonyl, C₁₋₆ haloalkylcarbonyl, C₁₋₆alkenylcarbonyl, C₁₋₆ haloalkenylcarbonyl, C₁₋₆ alkynyl-carbonyl, C₁₋₆haloalkynylcarbonyl, C₁₋₆alkoxycarbonyl, aminocarbonyl, C₁₋₆ alkylaminocarbonyl, C₁₋₆haloalkylaminocarbonyl, C₁₋₆ hydroxyalkylaminocarbonyl, C₂₋₁₂ dialkylamino-carbonyl, C₂₋₆ di(haloalkyl)aminocarbonyl, C₂₋₆ alkenylaminocarbonyl, C₂₋₆ alkynylaminocarbonyl, phenyaminocarbonyl, halophenylaminocarbonyl, C₁₋₆ alkyl-thiocarbonyl, C₃₋₆ cycloalkylcarbonyl, C₄₋₁₂ cycloalkylalkyl-carbonyl, C₃₋₆ cycloalkyl-thiocarbonyl, C₄₋₁₂ cycloalkylalkyl-thiocarbonyl, C₁₋₆haloalkyl-thiocarbonyl, C₁₋₆ alkylamino-thiocarbonyl, C₃₋₆ cycloalkylamino-carbonyl, C₄₋₁₂ cycloalkylalkylamino-carbonyl, C₃₋₆ cycloalkylamino-thiocarbonyl, C₄₋₁₂ cycloalkylalkyl-aminothiocarbonyl, C₁₋₆haloalkylamino-thiocarbonyl, C₂₋₁₂ dialkylamino-thiocarbonyl, C₃₋₆ cyclo-alkyloxy-carbonyl, C₄₋₁₂ cycloalkylalkyloxy-carbonyl, C₁₋₆haloalkoxy-carbonyl, C₁₋₆alkylsulfonyl, C₁₋₆ haloalkylsulfonyl, phenylsulfonyl, phenyl-C₁₋₆ alkyl, pyridin-2-yl-C₁₋₆ alkyl, 3-pyridyl-C₁₋₆ alkyl, 4-pyridyl-C₁₋₆ alkyl, phenylcarbonyl, 2-pyridylcarbonyl, 3-pyridylcarbonyl, 4-pyridyl-carbonyl, phenyl-thiocarbonyl, 2-pyridyl-thiocarbonyl, 3-pyridyl-thiocarbonyl, 4-pyridyl-thiocarbonyl, phenyl-C₁₋₆ alkylcarbonyl, 2-pyridyl-C₁₋₆alkylcarbonyl, 3-pyridyl-C₁₋₆ alkylcarbonyl, 4-pyridyl-C₁₋₆ alkylcarbonyl, phenyl-C₁₋₆alkyl-thiocarbonyl, 2-pyridyl-C₁₋₆alkyl-thiocarbonyl, 3-pyridyl-C₁₋₆alkyl-thiocarbonyl, 4-pyridyl-C₁₋₆alkyl-thiocarbonyl, phenyl-oxycarbonyl, 2-pyridyloxycarbonyl, 3-pyridyl-oxycarbonyl, 4-pyridyloxycarbonyl, phenyl-C₁₋₆ alkyl-oxy-carbonyl, 2-pyridyl-C₁₋₆ alkyloxy-carbonyl, 3-pyridyl-C₁₋₆alkyloxy-carbonyl, 4-pyridyl-C₁₋₆ alkyloxy-carbonyl, phenyl-aminocarbonyl, 2-pyridyl-aminocarbonyl, 3-pyridyl-amino-carbonyl, 4-pyridyl-aminocarbonyl, phenyl-amino-thiocarbonyl, 2-pyridylamino-thiocarbonyl, 3-pyridylamino-thiocarbonyl, 4-pyridylamino-thiocarbonyl, phenyl-C₁₋₆ alkylamino-carbonyl, halophenyl-C₁₋₆alkylamino-carbonyl, pyridin-2-yl-C₁₋₆alkylamino-carbonyl, 3-pyridyl-C₁₋₆ alkyl-amino-carbonyl, 4-pyridyl-C₁₋₆ alkylamino-carbonyl, phenyl-C₁₋₆alkylamino-thiocarbonyl, 2-pyridyl-C₁₋₆alkylamino-thiocarbonyl, 3-pyridyl-C₁₋₆alkylamino-thiocarbonyl or 4-pyridyl-C₁₋₆ alkylamino-thiocarbonyl, more preferably represents hydrogen, C₁₋₆ alkyl, C₁₋₆ haloalkyl, C₁₋₆ alkylcarbonyl, C₁₋₆haloalkylcarbonyl, C₁₋₆alkoxycarbonylC₁₋₆alkylaminocarbonyl, C₁₋₆ haloalkylaminocarbonyl, C₂₋₁₂ dialkylaminocarbonyl, C₂₋₆ alkenylaminocarbonyl, C₂₋₆ alkynyl-aminocarbonyl, phenylaminocarbonyl, halophenylaminocarbonyl, C₁₋₆ alkylamino-thiocarbonyl, C₃₋₆ cycloalkylamino-carbonyl, C₄₋₁₂ cycloalkylalkylamino-carbonyl, phenyl-C₁₋₆ alkyl, pyridin-2-yl-C₁₋₆ alkyl, phenyl-C₁₋₆ alkylamino-carbonyl, halophenyl-C₁₋₆ alkylamino-carbonyl, pyridin-2-yl-C₁₋₆alkylamino-carbonyl.

Embodiment F: Compounds having the structure (I-f-1), wherein the chemical groups A, R¹, Q, X, Y, m, n, and W′ are as defined herein and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆ alkylsulfinyl, C₁₋₆alkylsulfonyl.

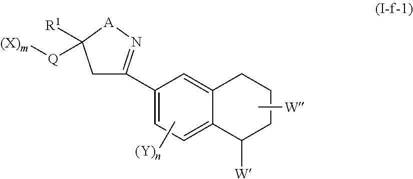

Embodiment F1: Compounds as defined in embodiment F, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment F2: Compounds as defined in embodiment F or F1, wherein A represents oxygen or CH₂, R¹ represents CF₃, and/or Y is H.

In these embodiments, W′ preferably represents amino, hydroxy, carbonylamino, C₁₋₆ alkyl-carbonylamino, C₁₋₆ alkylcarbonyl-C₁₋₆ alkyl-carbonylamino, C₁₋₆ alkylsulfenyl-C₁₋₆alkyl-carbonyl-amino, C₁₋₆alkylsulfenyl-C₁₋₆alkyl-carbonylamino, C₁₋₆ alkylsulfinyl-C₁₋₆alkyl-carbonylamino, C₁₋₆ alkylsulfonyl-C₁₋₆ alkyl-carbonylamino, C₁₋₆ haloalkyl-carbonylamino, cyano-C₁₋₆ alkyl-carbonylamino C₁₋₆ alkenyl-carbonylamino, C₁₋₆haloalkenyl-carbonylamino, C₁₋₆ alkynyl-carbonylamino, C₁₋₆ haloalkynyl-carbonylamino, C₁₋₆ alkoxy-carbonylamino, C₁₋₆haloalkoxy-carbonylamino, cyano-C₁₋₆ alkoxy-carbonylamino, C₁₋₆ alkoxy-C₁₋₆ alkylamino-carbonylamino, C₁₋₆ alkoxy-C₁₋₆ alkoxy-carbonylamino, amino-carbonylamino, C₁₋₆ alkylamino-carbonylamino, C₁₋₆ haloalkylamino-carbonylamino, hydroxyC₁₋₆alkylamino-carbonylamino, cyano-C₁₋₆ alkylamino-carbonylamino, C₂₋₁₂ dialkylamino-carbonylamino, C₂₋₁₂ dialkylamino-C₁₋₆alkyl-carbonylamino, C₂₋₆ di(haloalkyl)amino-carbonylamino, C₂₋₆ alkenylamino-carbonyl-amino, C₂₋₆ alkynylamino-carbonylamino, C₁₋₆ alkoxy-amino-carbonylamino, C₁₋₆ alkyl-sulfonylamino, C₁₋₆alkenyloxy-amino-carbonylamino, C₁₋₆alkyl-thiocarbonylamino, C₃₋₆ cyclo-alkyl-carbonylamino, C₃₋₆ halocycloalkyl-carbonylamino, C₄₋₁₂ cycloalkylalkyl-carbonylamino, C₃₋₆ cycloalkyl-thiocarbonylamino, C₄₋₁₂ cycloalkylalkyl-thiocarbonylamino, C₁₋₆haloalkyl-thiocarbonylamino, C₁₋₆alkylamino-thiocarbonylamino, C₃₋₆ cycloalkylamino-carbonylamino, C₃₋₆ halocycloalkylamino-carbonylamino, C₄₋₁₂ cycloalkylalkylamino-carbonylamino, C₃₋₆ cyclo-alkylamino-thiocarbonylamino, C₄₋₁₂ cycloalkylalkylaminothiocarbonylamino, C₁₋₆ haloalkylamino-thiocarbonylamino, C₂₋₁₂ dialkylamino-thiocarbonylamino, C₃₋₆cycloalkyloxy-carbonylamino, C₄₋₁₂ cycloalkylalkyloxy-carbonylamino, C₁₋₆ haloalkoxy-carbonylamino, C₁₋₆ alkylsulfonylamino, C₁₋₆ haloalkylsulfonylamino, C₁₋₆ alkylsulfonylamino, phenylsulfonylamino, phenyl-C₁₋₆ alkylamino, 2-pyridyl-C₁₋₆ alkylamino, 3-pyridyl-C₁₋₆ alkylamino, 4-pyridyl-C₁₋₆ alkylamino, phenyl-carbonyl-amino, halo-phenyl-carbonylamino, azetidine-1-yl-carbonylamino, pyrrolidine-1-yl-carbonylamino, N-methyl-pyrrolidine-2-yl-carbonylamino, furan-2-yl-carbonylamino, trifluoromethylphenyl-carbonylamino, phenylamino-carbonylamino, halophenylamino-carbonylamino, trifluoromethylphenylamino-carbonylamino, 2-pyridyl-carbonylamino, 3-pyridyl-carbonylamino, 4-pyridyl-carbonylamino, phenyl-thiocarbonylamino, 2-pyridyl-thiocarbonylamino, 3-pyridyl-thiocarbonylamino, 4-pyridyl-thiocarbonylamino, phenyl-C₁₋₆ alkyl-carbonylamino, 2-pyridyl-C₁₋₆alkyl-carbonylamino, 3-pyridyl-C₁₋₆alkylcarbonyl, 4-pyridyl-C₁₋₆ alkylcarbony, phenyl-C₁₋₆ alkyl-thiocarbonyl, 2-pyridyl-C₁₋₆ alkyl-thiocarbonyl, 3-pyridyl-C₁₋₆alkyl-thiocarbonylamino, 4-pyridyl-C₁₋₆ alkyl-thiocarbonylamino, phenyl-oxy-carbonylamino, 2-pyridyloxy-carbonylamino, 3-pyridyloxy-carbonylamino, 4-pyridyloxy-carbonylamino, phenyl-C₁₋₆alkyloxy-carbonylamino, 2-pyridyl-C₁₋₆alkyloxy-carbonylamino, 3-pyridyl-C₁₋₆ alkyloxy-carbonylamino, 4-pyridyl-C₁₋₆ alkyloxy-carbonylamino, phenyl-amino-carbonylamino, 2-pyridyl-amino-carbonylamino, 3-pyridyl-amino-carbonylamino, 4-pyridyl-amino-carbonylamino, phenyl-amino-thiocarbonyl-amino, 2-pyridylamino-thiocarbonylamino, 3-pyridylamino-thiocarbonylamino, 4-pyridylamino-thiocarbonylamino, phenyl-C₁₋₆ alkylamino-carbonylamino, 2-pyridyl-C₁₋₆ alkylamino-carbonyl-amino, 3-pyridyl-C₁₋₆ alkylamino-carbonylamino, 4-pyridyl-C₁₋₆ alkylamino-carbonylamino, phenyl-C₁₋₆ alkylamino-thiocarbonylamino, 2-pyridyl-C₁₋₆alkylamino-thiocarbonylamino, 3-pyridyl-C₁₋₆ alkylamino-thiocarbonylamino or 4-pyridyl-C₁₋₆ alkylamino-thiocarbonylamino, 1,2,3-triazole-4-trimethylsilyl-1-yl, 1,2,3-triazole-1-yl, succinimide-1-yl, —N₃, phthalimide-2-yl, C₁₋₆ alkylcarbonyloxy, more preferably represents amino, hydroxy, carbonylamino, C₁₋₆ alkyl-sulfonylamino, C₁₋₆ alkylamino-carbonylamino, C₁₋₆ alkyl-thiocarbonylamino, C₁₋₆ alkyl-carbonyl-amino, C₁₋₆ alkylcarbonyl-C₁₋₆ alkyl-carbonylamino, alkylsulfenyl-C₁₋₆ alkyl-carbonylamino, C₁₋₆ alkylsulfenyl-C₁₋₆alkyl-carbonylamino, C₁₋₆ alkylsulfinyl-C₁₋₆ alkyl-carbonylamino, C₁₋₆ alkyl-sulfonyl-C₁₋₆alkyl-carbonylamino, C₁₋₆ haloalkyl-carbonylamino, cyano-C₁₋₆ alkyl-carbonylamino, C₁₋₆ alkenyl-carbonylamino, C₁₋₆ alkynyl-carbonylamino, C₁₋₆ alkoxy-carbonylamino, C₁₋₆haloalkoxy-carbonylamino, cyano-C₁₋₆alkoxy-carbonylamino, C₁₋₆ alkoxy C₁₋₆ alkylamino-carbonylamino, C₁₋₆ alkoxy-C₁₋₆ alkoxy-carbonylamino, amino-carbonylamino, C₁₋₆ alkylamino-carbonylamino, C₁₋₆haloalkylamino-carbonylamino, hydroxy-C₁₋₆-alkylamino-carbonylamino, cyano-C₁₋₆ alkylamino-carbonylamino, C₂₋₁₂ dialkylamino-carbonylamino, C₂₋₁₂ dialkylamino-C₁₋₆ alkyl-carbonylamino, C₂₋₆ alkynylamino-carbonylamino, C₁₋₆ alkoxy-amino-carbonylamino, C₁₋₆ alkenyloxy-amino-carbonylamino, C₃₋₆ cycloalkyl-carbonylamino, C₃₋₆ halocycloalkyl-carbonylamino, C₄₋₁₂ cycloalkylalkyl-carbonylamino, C₁₋₆ alkylamino-thiocarbonylamino, C₃₋₆ cycloalkylamino-carbonylamino, C₃₋₆ halocycloalkylamino-carbonylamino, C₄₋₁₂ cycloalkyl-alkylamino-carbonylamino, C₁₋₆haloalkylsulfonylamino, phenyl-carbonylamino, halo-phenyl-carbonylamino, azetidine-1-yl-carbonylamino, pyrrolidine-1-yl-carbonylamino, N-methyl-pyrrolidine-2-yl-carbonylamino, furan-2-yl-carbonylamino, tirfluoromethylphenyl-carbonylamino, phenylamino-carbonylamino, halophenylamino-carbonylamino, trifluoromethylphenylamino-carbonylamino, 1,2,3-triazole-4-trimethylsilyl-1-yl, 1,2,3-triazole-1-yl, succinimide-1-yl, —N₃, phthalimide-2-yl, C₁₋₆ alkylcarbonyloxy, most preferably represents C₁₋₆ haloalkyl-carbonylamino, C₁₋₆ alkylamino-carbonylamino, C₁₋₆ haloalkylamino-carbonylamino, C₁₋₆ alkylamino-carbonyl-amino and C₁₋₆haloalkylamino-carbonylamino.

Embodiment G: Compounds having the structure (I-g-1) or (I-g-2), wherein the chemical groups A, R¹, Q, X, Y, m, n, and W′ are as defined herein and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆ alkyl-sulfinyl, C₁₋₆alkylsulfonyl

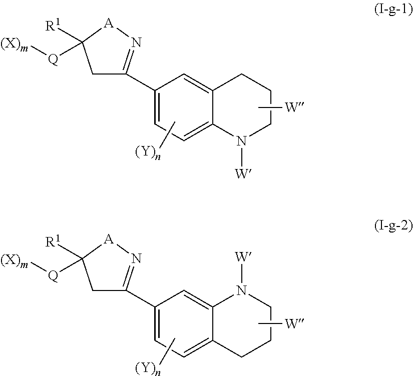

Embodiment G1: Compounds as defined in embodiment G, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆alkoxy, and C₁₋₆haloalkyl.

Embodiment G2: Compounds as defined in embodiment G or G1, wherein A represents oxygen or CH₂, R¹ represents CF₃, and Y is H.

In these embodiments, W′ preferably represents hydrogen, C₁₋₆ alkyl, C₁₋₆ alkylcarbonyl, C₁₋₆haloalkylcarbonyl, C₁₋₆alkenylcarbonyl, C₁₋₆haloalkenylcarbonyl, C₁₋₆ alkynylcarbonyl, C₁₋₆haloalkynylcarbonyl, C₁₋₆alkoxycarbonyl, aminocarbonyl, C₁₋₆ alkylaminocarbonyl, C₁₋₆haloalkylaminocarbonyl, C₁₋₆ hydroxyalkylaminocarbonyl, C₂₋₁₂ dialkylamino-carbonyl, C₂₋₆ di(haloalkyl)aminocarbonyl, C₂₋₆alkenylaminocarbonyl, C₂₋₆ alkynylaminocarbonyl, C₁₋₆ alkyl-thiocarbonyl, C₃₋₆ cycloalkylcarbonyl, C₄₋₁₂ cycloalkylalkyl-carbonyl, C₃₋₆ cycloalkyl-thiocarbonyl, C₄₋₁₂ cycloalkylalkyl-thiocarbonyl, C₁₋₆haloalkyl-thiocarbonyl, C₁₋₆ alkylamino-thiocarbonyl, C₂₋₆cycloalkylamino-carbonyl, C₄₋₁₂ cycloalkylalkylamino-carbonyl, C₃₋₆ cycloalkylamino-thiocarbonyl, C₄₋₁₂ cycloalkylalkylaminothiocarbonyl, C₁₋₆haloalkylamino-thiocarbonyl, C₂₋₁₂ dialkylamino-thiocarbonyl, C₂₋₆cycloalkyloxy-carbonyl, C₄₋₁₂cycloalkylalkyloxy-carbonyl, C₁₋₆haloalkoxy-carbonyl, C₁₋₆ alkylsulfonyl, C₁₋₆haloalkylsulfonyl, phenylsulfonyl, phenyl-C₁₋₆ alkyl, 2-pyridyl-C₁₋₆ alkyl, 3-pyridyl-C₁₋₆alkyl, 4-pyridyl-C₁₋₆ alkyl, phenylcarbonyl, 2-pyridylcarbonyl, 3-pyridylcarbonyl, 4-pyridylcarbonyl, phenyl-thiocarbonyl, 2-pyridyl-thiocarbonyl, 3-pyridyl-thiocarbonyl, 4-pyridyl-thiocarbonyl, phenyl-C₁₋₆ alkylcarbonyl, 2-pyridyl-C₁₋₆alkylcarbonyl, 3-pyridyl-C₁₋₆alkylcarbonyl, 4-pyridyl-C₁₋₆ alkylcarbony, phenyl-C₁₋₆alkyl-thiocarbonyl, 2-pyridyl-C₁₋₆alkyl-thiocarbonyl, 3-pyridyl-C₁₋₆alkyl-thiocarbonyl, 4-pyridyl-C₁₋₆alkyl-thiocarbonyl, phenyl-oxycarbonyl, 2-pyridyloxycarbonyl, 3-pyridyl-oxycarbonyl, 4-pyridyloxycarbonyl, phenyl-C₁₋₆ alkyloxy-carbonyl, 2-pyridyl-C₁₋₆alkyloxy-carbonyl, 3-pyridyl-C₁₋₆alkyloxy-carbonyl, 4-pyridyl-C₁₋₆ alkyloxy-carbonyl, phenyl-aminocarbonyl, 2-pyridyl-amino-carbonyl, 3-pyridyl-aminocarbonyl, 4-pyridyl-aminocarbonyl, phenyl-amino-thiocarbonyl, 2-pyridylamino-thiocarbonyl, 3-pyridylamino-thiocarbonyl, 4-pyridylamino-thiocarbonyl, phenyl-C₁₋₆ alkylamino-carbonyl, 2-pyridyl-C₁₋₆ alkylamino-carbonyl, 3-pyridyl-C₁₋₆alkylamino-carbonyl, 4-pyridyl-C₁₋₆ alkylamino-carbonyl, phenyl-C₁₋₆alkylamino-thiocarbonyl, 2-pyridyl-C₁₋₆ alkyl-amino-thiocarbonyl, 3-pyridyl-C₁₋₆ alkylamino-thiocarbonyl or 4-pyridyl-C₁₋₆alkylamino-thiocarbonyl, more preferably represents hydrogen, C₁₋₆ alkyl, C₁₋₆alkylcarbonyl, C₁₋₆ alkylamino-carbonyl, C₁₋₆ haloalkylaminocarbonyl, C₂₋₆ alkenylaminocarbonyl, C₂₋₆ alkynylaminocarbonyl, C₃₋₆ cycloalkylamino-carbonyl, C₄₋₁₂ cycloalkylalkylamino-carbonyl.

Embodiment H: Compounds having the following structure (I-h-1) or (I-h-2), wherein the chemical groups A, R¹, Q, X, Y, m, n, W′ and U are as defined herein, and wherein W″ is selected among hydrogen, halogen, hydroxy, thiol, cyano, C₁₋₆ alkyl, C₁₋₆ haloalkyl, C₁₋₆ alkoxy, C₁₋₆ alkyl-sulfenyl, C₁₋₆alkylsulfinyl, C₁₋₆alkylsulfonyl.

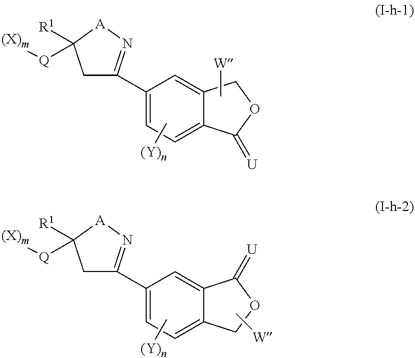

Embodiment H1: Compounds as defined in embodiment H, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment H2: Compounds as defined in embodiment H or H1, wherein A represents oxygen or CH₂, R¹ represents CF₃, and Y is H and/or U stands for O, C₁₋₆ alkyl-N, pyridin-2-yl-C₁₋₆ alkyl-N or H—N.

Embodiment I: Compounds having the structure (I-i-1) or (I-i-2) wherein the chemical groups A, R¹, Q, X, Y, m, n, W′ and U are as defined herein, and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆alkyl-sulfinyl, C₁₋₆alkylsulfonyl.

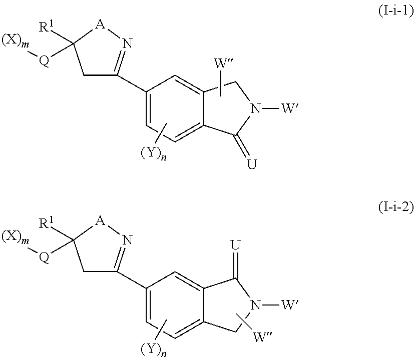

Embodiment I1: Compounds as defined in embodiment I, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment I2: Compounds as defined in embodiment I or I1, wherein A represents oxygen or CH₂, R¹ represents CF₃, and Y is H and/or U stands for O or C₁₋₆ alkyl-carbonyl-N.

In these embodiments, W′ preferably represents hydrogen; formyl; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₆ alkynyl, C₃₋₈ cycloalkyl, C₄₋₁₂ (total carbon number) alkylcycloalkyl, C₄₋₁₂ (total carbon number) cycloalkylalkyl, phenyl, halophenyl, pyrimidin-2-yl, C₁₋₆ haloalkyl, C₁₋₆ alkylimino, C₁₋₆ haloalkylimino, C₁₋₆ alkyl-carbonyl, C₁₋₆ alkenyl-carbonyl, C₁₋₆ alkynyl-carbonyl, C₁₋₆ haloalkyl-carbonyl, C₁₋₆ alkoxy-carbonyl, C₁₋₆ haloalkoxy-carbonyl, aminocarbonyl, C₁₋₆ alkylamino-carbonyl, C₁₋₆ haloalkylamino-carbonyl, C₁₋₆ hydroxyalkylamino-carbonyl, C₂₋₁₂ (total carbon number) dialkyl-amino-carbonyl, C₂₋₆ (total carbon number) di(haloalkyl)aminocarbonyl, C₂₋₆ alkenylamino-carbonyl, C₂₋₆ alkynylamino-carbonyl, C₃₋₆ cycloalkyl-carbonyl, C₄₋₁₂ (total carbon number) cyclo-alkylalkyl-carbonyl, C₃₋₆ cycloalkylamino-carbonyl, C₄₋₁₂ (total carbon number) cycloalkylalkyl-amino-carbonyl, C₃₋₆cycloalkyloxy-carbonyl, C₄₋₁₂ (total carbon number) cycloalkylalkyl-oxy-carbonyl, C₁₋₆ haloalkoxy-carbonyl, phenyl-C₁₋₆ alkyl, halophenyl-C₁₋₆ alkyl, 2-pyridyl-C₁₋₆ alkyl, 3-pyridyl-C₁₋₆ alkyl, 4-pyridyl-C₁₋₆ alkyl, phenylcarbonyl, 2-pyridylcarbonyl, 3-pyridyl-carbonyl, 4-pyridylcarbonyl, phenyl-C₁₋₆alkylcarbonyl, 2-pyridyl-C₁₋₆ alkylcarbonyl, 3-pyridyl-C₁₋₆ alkylcarbonyl, 4-pyridyl-C₁₋₆alkylcarbony, phenyloxy-carbonyl, 2-pyridyloxy-carbonyl, 3-pyridyloxy-carbonyl, 4-pyridyloxy-carbonyl, phenyl-C₁₋₆ alkyloxy-carbonyl, 2-pyridyl-C₁₋₆ alkyl-oxy-carbonyl, 3-pyridyl-C₁₋₆alkyloxy-carbonyl, 4-pyridyl-C₁₋₆alkyloxy-carbonyl, phenyl-amino-carbonyl, 2-pyridyl-aminocarbonyl, 3-pyridyl-aminocarbonyl, 4-pyridyl-aminocarbonyl, phenyl-C₁₋₆alkylamino-carbonyl, 2-pyridyl-C₁₋₆ alkylamino-carbonyl, 3-pyridyl-C₁₋₆alkylamino-carbonyl or 4-pyridyl-C₁₋₆alkylamino-carbonyl, more preferably represents hydrogen, C₁₋₆ alkyl, C₂₋₁₂ (total carbon number) alkoxyalkyl, C₂₋₆ alkenyl, C₃₋₈ cycloalkyl, phenyl, halophenyl, pyrimidin-2-yl, C₁₋₆haloalkyl, C₁₋₆ alkyl-carbonyl, phenyl-C₁₋₆ alkyl, halophenyl-C₁₋₆ alkyl, 2-pyridyl-C₁₋₆alkyl.

These embodiments do not include compounds nos. 1-50, 1-52, and 1-52 as defined herein.

Embodiment J: Compounds having the structure (I-j-1), wherein the chemical groups A, R¹, Q, X, Y, m, n, W′ and U are as defined herein, and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆alkylsulfinyl, C₁₋₆alkylsulfonyl.

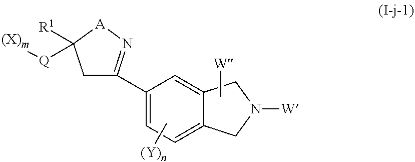

Embodiment J1: Compounds as defined in embodiment J, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment J2: Compounds as defined in embodiment J or J1, wherein A represents oxygen or CH₂, R¹ represents CF₃, and Y is H.

In these embodiments, W′ preferably represents hydrogen; formyl; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₆ alkynyl, C₃₋₈ cycloalkyl, C₄₋₁₂ (total carbon number) alkylcycloalkyl, C₄₋₁₂ (total carbon number) cycloalkylalkyl, C₁₋₆haloalkyl, C₁₋₆alkylimino, C₁₋₆haloalkylimino, C₁₋₆ alkyl-carbonyl, C₁₋₆ alkenyl-carbonyl, C₁₋₆ alkynyl-carbonyl, C₁₋₆ haloalkyl-carbonyl, C₁₋₆ alkoxy-carbonyl, C₁₋₆ haloalkoxy-carbonyl, aminocarbonyl, C₁₋₆ alkylamino-carbonyl, C₁₋₆haloalkylamino-carbonyl, C₁₋₆hydroxyalkylamino-carbonyl, C₂₋₁₂ (total carbon number) dialkylamino-carbonyl, C₂₋₆ (total carbon number) di(haloalkyl)aminocarbonyl, C₂₋₆ alkenylamino-carbonyl, C₂₋₆ alkynylamino-carbonyl, C₃₋₆ cyclo-alkyl-carbonyl, C₄₋₁₂ (total carbon number) cycloalkylalkyl-carbonyl, C₃₋₆ cycloalkylamino-carbonyl, C₄₋₁₂ (total carbon number) cycloalkylalkylamino-carbonyl, C₃₋₆ cycloalkyloxy-carbonyl, C₄₋₁₂ (total carbon number) cycloalkylalkyloxy-carbonyl, C₁₋₆ haloalkoxy-carbonyl, phenyl-C₁₋₆alkyl, 2-pyridyl-C₁₋₆ alkyl, 3-pyridyl-C₁₋₆ alkyl, 4-pyridyl-C₁₋₆alkyl, phenylcarbonyl, 2-pyridylcarbonyl, 3-pyridylcarbonyl, 4-pyridylcarbonyl, phenyl-C₁₋₆ alkylcarbonyl, 2-pyridyl-C₁₋₆ alkylcarbonyl, 3-pyridyl-C₁₋₆alkylcarbonyl, 4-pyridyl-C₁₋₆alkylcarbony, phenyloxy-carbonyl, 2-pyridyloxy-carbonyl, 3-pyridyloxy-carbonyl, 4-pyridyloxy-carbonyl, phenyl-C₁₋₆ alkyl-oxy-carbonyl, 2-pyridyl-C₁₋₆alkyloxy-carbonyl, 3-pyridyl-C₁₋₆alkyloxy-carbonyl, 4-pyridyl-C₁₋₆ alkyloxy-carbonyl, phenyl-aminocarbonyl, 2-pyridyl-aminocarbonyl, 3-pyridyl-amino-carbonyl, 4-pyridyl-aminocarbonyl, phenyl-C₁₋₆ alkylamino-carbonyl, 2-pyridyl-C₁₋₆alkylamino-carbonyl, 3-pyridyl-C₁₋₆ alkylamino-carbonyl or 4-pyridyl-C₁₋₆alkylamino-carbonyl, more preferably represents hydrogen; C₁₋₆ alkyl-carbonyl, phenyl-C₁₋₆ alkyl, or 2-pyridyl-C₁₋₆ alkyl.

Embodiment K: Compounds having the structure (I-k-1), wherein the chemical groups A, R¹, Q, X, Y, m, n, W′ and U are as defined herein, and wherein W″ is selected among hydrogen, halogen, hydroxy, thiol, cyano, C₁₋₆ alkyl, C₁₋₆haloalkyl, C₁₋₆ alkoxy, C₁₋₆ alkylsulfenyl, C₁₋₆alkylsulfinyl, C₁₋₆alkylsulfonyl.

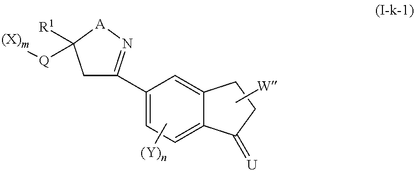

Embodiment K1: Compounds as defined in embodiment K, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆haloalkyl.

Embodiment K2: Compounds as defined in embodiment K or K1, wherein A represents oxygen or CH₂, R¹ represents CF₃, and Y is H and/or U stands for O, hydroxy-N, C₁₋₆ alkoxy-N, C₁₋₆ haloalkoxy-N, C₁₋₆ alkylamino-N, C₂₋₁₂ di(alkyl)amino-N, 2-pyridyl-C₁₋₆ alkoxy-N, C₁₋₆ alkyl-carbonylamino-N, or C₁₋₆haloalkylamino-N.

Embodiment L: Compounds having the structure (I-1-1), wherein the chemical groups A, R¹, Q, X, Y, m, n, W′ and U are as defined herein, and wherein W″ is selected among hydrogen, halogen, hydroxy, thiol, cyano, C₁₋₆alkyl, C₁₋₆haloalkyl, C₁₋₆alkoxy, C₁₋₆alkylsulfenyl, C₁₋₆ alkylsulfinyl, C₁₋₆alkylsulfonyl.

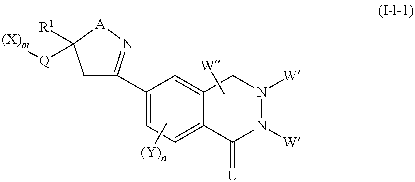

Embodiment L1: Compounds as defined in embodiment L, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆alkoxy, and C₁₋₆haloalkyl.

Embodiment L2: Compounds as defined in embodiment L or L1, wherein A represents oxygen or CH₂, R¹ represents CF₃, Y is H and/or U stands for O.

In these embodiments, W′ preferably represents hydrogen; formyl; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₆ alkynyl, C₃₋₈ cycloalkyl, C₄₋₁₂ (total carbon number) alkylcycloalkyl, C₄₋₁₂ (total carbon number) cycloalkylalkyl, C₁₋₆haloalkyl, C₁₋₆ alkylimino, C₁₋₆haloalkylimino, C₁₋₆ alkenyl-carbonyl, C₁₋₆ alkynyl-carbonyl, C₁₋₆haloalkyl-carbonyl, C₁₋₆ alkoxy-carbonyl, C₁₋₆ haloalkoxy-carbonyl, aminocarbonyl, C₁₋₆ alkyl-amino-carbonyl, C₁₋₆ haloalkylamino-carbonyl, C₁₋₆ hydroxyalkylamino-carbonyl, C₂₋₁₂ (total carbon number) dialkylamino-carbonyl, C₂₋₆ (total carbon number) di(haloalkyl)aminocarbonyl, C₂₋₆ alkenylamino-carbonyl, C₂₋₆ alkynylamino-carbonyl, C₃₋₆ cycloalkyl-carbonyl, C₄₋₁₂ (total carbon number) cycloalkylalkyl-carbonyl, C₃₋₆ cycloalkylamino-carbonyl, C₄₋₁₂ (total carbon number) cycloalkylalkylamino-carbonyl, C₃₋₆ cycloalkyloxy-carbonyl, C₄₋₁₂ (total carbon number) cycloalkylalkyloxy-carbonyl, C₁₋₆haloalkoxy-carbonyl, phenyl-C₁₋₆ alkyl, 2-pyridyl-C₁₋₆ alkyl, 3-pyridyl-C₁₋₆ alkyl, 4-pyridyl-C₁₋₆ alkyl, phenylcarbonyl, 2-pyridylcarbonyl, 3-pyridylcarbonyl, 4-pyridylcarbonyl, phenyl-C₁₋₆alkylcarbonyl, 2-pyridyl-C₁₋₆alkylcarbonyl, 3-pyridyl-C₁₋₆ alkyl-carbonyl, 4-pyridyl-C₁₋₆alkylcarbony, phenyloxy-carbonyl, 2-pyridyloxy-carbonyl, 3-pyridyloxy-carbonyl, 4-pyridyloxy-carbonyl, phenyl-C₁₋₆alkyloxy-carbonyl, 2-pyridyl-C₁₋₆ alkyl-oxy-carbonyl, 3-pyridyl-C₁₋₆ alkyloxy-carbonyl, 4-pyridyl-C₁₋₆alkyloxy-carbonyl, phenyl-amino-carbonyl, 2-pyridyl-aminocarbonyl, 3-pyridyl-aminocarbonyl, 4-pyridyl-aminocarbonyl, phenyl-C₁₋₆ alkylamino-carbonyl, 2-pyridyl-C₁₋₆alkylamino-carbonyl, 3-pyridyl-C₁₋₆alkylamino-carbonyl or 4-pyridyl-C₁₋₆alkylamino-carbonyl, more preferably represents hydrogen or C₁₋₆alkyl

Embodiment M: Compounds having the following structure (I-m-1), wherein the chemical groups A, R¹, Q, X, Y, m, n, W and U are as defined herein, and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆alkyl-sulfinyl, C₁₋₆ alkylsulfonyl.

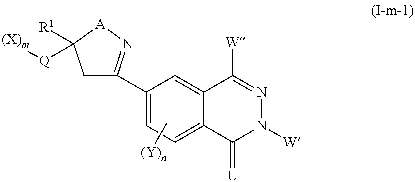

Embodiment M1: Compounds as defined in embodiment M, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment M2: Compounds as defined in embodiment M or M1, wherein A represents oxygen or CH₂, R¹ represents CF₃, Y is H and/or U stands for O.

In these embodiments, W′ preferably represents hydrogen; formyl; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₆ alkynyl, C₃₋₈ cycloalkyl, C₄₋₁₂ (total carbon number) alkylcycloalkyl, C₄₋₁₂ (total carbon number) cycloalkylalkyl, C₁₋₆ haloalkyl, C₁₋₆ alkylimino, C₁₋₆haloalkylimino, C₁₋₆ alkenyl-carbonyl, C₁₋₆ alkynyl-carbonyl, C₁₋₆ haloalkyl-carbonyl, C₁₋₆ alkoxy-carbonyl, C₁₋₆haloalkoxy-carbonyl, aminocarbonyl, C₁₋₆ alkyl-amino-carbonyl, C₁₋₆ haloalkylamino-carbonyl, C₁₋₆ hydroxyalkylamino-carbonyl, C₂₋₁₂ (total carbon number) dialkylamino-carbonyl, C₂₋₆ (total carbon number) di(haloalkyl)aminocarbonyl, C₂₋₆alkenylamino-carbonyl, C₂₋₆ alkynylamino-carbonyl, C₃₋₆ cycloalkyl-carbonyl, C₄₋₁₂ (total carbon number) cycloalkylalkyl-carbonyl, C₃₋₆ cycloalkylamino-carbonyl, C₄₋₁₂ (total carbon number) cycloalkylalkylamino-carbonyl, C₃₋₆ cycloalkyloxy-carbonyl, C₄₋₁₂ (total carbon number) cycloalkylalkyloxy-carbonyl, C₁₋₆ haloalkoxy-carbonyl, phenyl-C₁₋₆ alkyl, 2-pyridyl-C₁₋₆ alkyl, 3-pyridyl-C₁₋₆alkyl, 4-pyridyl-C₁₋₆alkyl, phenylcarbonyl, 2-pyridylcarbonyl, 3-pyridylcarbonyl, 4-pyridylcarbonyl, phenyl-C₁₋₆alkylcarbonyl, 2-pyridyl-C₁₋₆alkylcarbonyl, 3-pyridyl-C₁₋₆ alkyl-carbonyl, 4-pyridyl-C₁₋₆ alkylcarbony, phenyloxy-carbonyl, 2-pyridyloxy-carbonyl, 3-pyridyloxy-carbonyl, 4-pyridyloxy-carbonyl, phenyl-C₁₋₆ alkyloxy-carbonyl, 2-pyridyl-C₁₋₆alkyl-oxy-carbonyl, 3-pyridyl-C₁₋₆ alkyloxy-carbonyl, 4-pyridyl-C₁₋₆ alkyloxy-carbonyl, phenyl-amino-carbonyl, 2-pyridyl-aminocarbonyl, 3-pyridyl-aminocarbonyl, 4-pyridyl-aminocarbonyl, phenyl-C₁₋₆alkylamino-carbonyl, 2-pyridyl-C₁₋₆alkylamino-carbonyl, 3-pyridyl-C₁₋₆alkylamino-carbonyl or 4-pyridyl-C₁₋₆alkylamino-carbonyl.

Embodiment N: Compounds having the structure (I-n-1), wherein the chemical groups A, R¹, Q, X, Y, m, n and W′ are as defined herein.

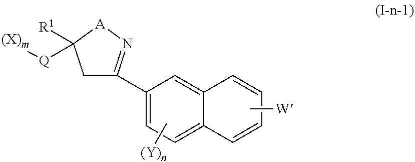

Embodiment N1: Compounds as defined in embodiment N, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆alkoxy, and C₁₋₆haloalkyl.

Embodiment N2: Compounds as defined in embodiment N or N1, wherein A represents oxygen or CH₂, R¹ represents CF₃, and Y is H.

In these embodiments, W′ preferably represents amino, hydroxy, C₁₋₆ alkyl-carbonylamino, C₁₋₆haloalkyl-carbonylamino, C₁₋₆alkenyl-carbonylamino, C₁₋₆haloalkenyl-carbonylamino, C₁₋₆ alkynyl-carbonylamino, C₁₋₆ haloalkynyl-carbonylamino, C₁₋₆ alkoxy-carbonylamino, amino-carbonylamino, C₁₋₆ alkylamino-carbonylamino, C₁₋₆ haloalkylamino-carbonylamino, C₁₋₆ hydroxyalkylamino-carbonylamino, C₂₋₁₂ dialkylamino-carbonylamino, C₂₋₆ di(haloalkyl)-amino-carbonylamino, C₂₋₆ alkenylamino-carbonylamino, C₂₋₆alkynylamino-carbonylamino, C₁₋₆alkyl-thiocarbonylamino, C₃₋₆ cycloalkyl-carbonylamino, C₄₋₁₂ cycloalkylalkyl-carbonylamino, C₃₋₆ cycloalkyl-thiocarbonylamino, C₄₋₁₂ cycloalkylalkyl-thiocarbonylamino, C₁₋₆ haloalkyl-thiocarbonylamino, C₁₋₆alkylamino-thiocarbonylamino, C₃₋₆ cycloalkylamino-carbonylamino, C₄₋₁₂ cycloalkylalkylamino-carbonylamino, C₃₋₆ cycloalkylamino-thiocarbonylamino, C₄₋₁₂ cyclo-alkylalkylaminothiocarbonylamino, C₁₋₆haloalkylamino-thiocarbonylamino, C₂₋₁₂ dialkylamino-thiocarbonylamino, C₃₋₆ cycloalkyloxy-carbonylamino, C₄₋₁₂ cycloalkylalkyloxy-carbonylamino, C₁₋₆haloalkoxy-carbonylamino, C₁₋₆alkylsulfonylamino, C₁₋₆haloalkylsulfonylamino, phenyl-sulfonylamino, phenyl-C₁₋₆ alkylamino, 2-pyridyl-C₁₋₆alkylamino, 3-pyridyl-C₁₋₆ allylamino, 4-pyridyl-C₁₋₆alkylamino, phenyl-carbonylamino, 2-pyridyl-carbonylamino, 3-pyridyl-carbonyl-amino, 4-pyridyl-carbonylamino, phenyl-thiocarbonylamino, 2-pyridyl-thiocarbonylamino, 3-pyridyl-thiocarbonylamino, 4-pyridyl-thiocarbonylamino, phenyl-C₁₋₆alkyl-carbonylamino, 2-pyridyl-C₁₋₆alkyl-carbonylamino, 3-pyridyl-C₁₋₆alkylcarbonyl, 4-pyridyl-C₁₋₆ alkylcarbony, phenyl-C₁₋₆alkyl-thiocarbonyl, 2-pyridyl-C₁₋₆alkyl-thiocarbonyl, 3-pyridyl-C₁₋₆alkyl-thiocarbonyl-amino, 4-pyridyl-C₁₋₆alkyl-thiocarbonylamino, phenyl-oxy-carbonylamino, 2-pyridyloxy-carbonyl-amino, 3-pyridyloxy-carbonylamino, 4-pyridyloxy-carbonylamino, phenyl-C₁₋₆ alkyloxy-carbonyl-amino, 2-pyridyl-C₁₋₆alkyloxy-carbonylamino, 3-pyridyl-C₁₋₆ alkyloxy-carbonylamino, 4-pyridyl-C₁₋₆ alkyloxy-carbonylamino, phenyl-amino-carbonylamino, 2-pyridyl-amino-carbonylamino, 3-pyridyl-amino-carbonylamino, 4-pyridyl-amino-carbonylamino, phenyl-amino-thiocarbonyl-amino, 2-pyridylamino-thiocarbonylamino, 3-pyridylamino-thiocarbonylamino, 4-pyridylamino-thiocarbonylamino, phenyl-C₁₋₆ alkylamino-carbonylamino, 2-pyridyl-C₁₋₆alkylamino-carbonyl-amino, 3-pydidyl-C₁₋₆alkylamino-carbonylamino, 4-pyridyl-C₁₋₆ alkylamino-carbonylamino, phenyl-C₁₋₆alkyl amino-thiocarbonylamino, 2-pyridyl-C₁₋₆alkylamino-thiocarbonylamino, 3-pydidyl-C₁₋₆alkylamino-thiocarbonylamino or 4-pyridyl-C₁₋₆alkylamino-thiocarbonylamino, more preferably represents amino, C₁₋₆alkyl-carbonylamino, C₁₋₆ alkoxy-carbonylaminoC₃₋₆cyclo-alkyl-carbonylamino.

Embodiment O: Compounds having the structure (I-o-1), wherein the chemical groups A, R¹, Q, X, Y, m, W′ and n are as defined herein, and wherein W″ is selected among hydrogen, halogen, hydroxy, thiol, cyano, C₁₋₆ alkyl, C₁₋₆ haloalkyl, C₁₋₆ alkoxy, C₁₋₆ alkylsulfenyl, C₁₋₆alkylsulfinyl, C₁₋₆alkylsulfonyl.

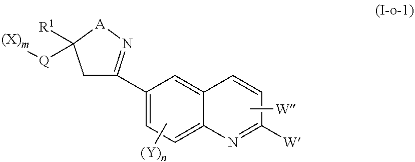

Embodiment O1: Compounds as defined in embodiment 0, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆ haloalkyl.

Embodiment O2: Compounds as defined in embodiment O or O1, wherein A represents oxygen or CH₂, R¹ represents CF₃, and Y is H.

In these embodiments, W′ preferably represents hydrogen; cyano, halogen. formyl; C₁₋₆ alkyl, C₂₋₁₂ (total carbon number) alkoxyalkyl, C₂₋₁₂ (total carbon number) haloalkoxyalkyl, C₂₋₆ alkenyl, C₂₋₆ alkynyl, C₃₋₈ cycloalkyl, C₄₋₁₂ (total carbon number) alkylcycloalkyl, C₄₋₁₂ (total carbon number) cycloalkylalkyl, C₁₋₆ haloalkyl, C₁₋₆ alkylimino, C₁₋₆haloalkylimino, C₁₋₆ alkenyl-carbonyl, C₁₋₆ alkynyl-carbonyl, C₁₋₆haloalkyl-carbonyl, C₁₋₆ alkoxy-carbonyl, C₁₋₆haloalkoxy-carbonyl, aminocarbonyl, C₁₋₆ alkylamino-carbonyl, C₁₋₆ haloalkylamino-carbonyl, C₁₋₆ hydroxyalkylamino-carbonyl, C₂₋₁₂ (total carbon number) dialkylamino-carbonyl, C₂₋₆ (total carbon number) di(haloalkyl)aminocarbonyl, C₂₋₆ alkenylamino-carbonyl, C₂₋₆ alkynylamino-carbonyl, C₃₋₆ cyclo-alkyl-carbonyl, C₄₋₁₂ (total carbon number) cycloalkylalkyl-carbonyl, C₃₋₆ cycloalkylamino-carbonyl, C₄₋₁₂ (total carbon number) cycloalkylalkylamino-carbonyl, C₃₋₆ cycloalkyloxy-carbonyl, C₄₋₁₂ (total carbon number) cycloalkylalkyloxy-carbonyl, C₁₋₆haloalkoxy-carbonyl, phenyl-C₁₋₆ alkyl, 2-pyridyl-C₁₋₆ alkyl, 3-pyridyl-C₁₋₆ alkyl, 4-pyridyl-C₁₋₆ alkyl, phenylcarbonyl, 2-pyridylcarbonyl, 3-pyridylcarbonyl, 4-pyridylcarbonyl, phenyl-C₁₋₆ alkylcarbonyl, 2-pyridyl-C₁₋₆alkylcarbonyl, 3-pyridyl-C₁₋₆ alkylcarbonyl, 4-pyridyl-C₁₋₆ alkylcarbony, phenyloxy-carbonyl, 2-pyridyloxy-carbonyl, 3-pyridyloxy-carbonyl, 4-pyridyloxy-carbonyl, phenyl-C₁₋₆ allyl-oxy-carbonyl, 2-pyridyl-C₁₋₆alkyloxy-carbonyl, 3-pyridyl-C₁₋₆alkyloxy-carbonyl, 4-pyridyl-C₁₋₆alkyloxy-carbonyl, phenyl-aminocarbonyl, 2-pyridyl-aminocarbonyl, 3-pyridyl-amino-carbonyl, 4-pyridyl-aminocarbonyl, phenyl-C₁₋₆ alkylamino-carbonyl, 2-pyridyl-C₁₋₆alkylamino-carbonyl, 3-pyridyl-C₁₋₆ alkylamino-carbonyl or 4-pyridyl-C₁₋₆ alkylamino-carbonyl.

Embodiment V: Compounds having the structure (I-v-1), wherein the chemical groups A, R¹, Q, X, Y, m, n, W′, and U are as defined herein, and wherein W″ is selected among hydrogen, halogen, hydroxy, thiol, cyano, C₁₋₆alkyl, C₁₋₆haloalkyl, C₁₋₆alkoxy, C₁₋₆alkylsulfenyl, C₁₋₆alkylsulfinyl, C₁₋₆ alkylsulfonyl.

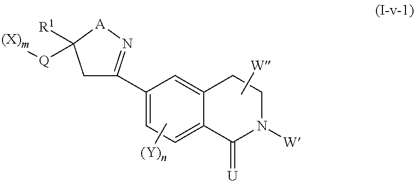

Embodiment V1: Compounds as defined in embodiment V, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆ alkoxy, and C₁₋₆haloalkyl.

Embodiment V2: Compounds as defined in embodiment V or V1, wherein A represents oxygen or CH₂, R¹ represents CF₃, and Y is H.

W′ preferably represents hydrogen.

Embodiment Z: Compounds having the structure (I-z-1), wherein the chemical groups A, R¹, Q, X, Y, m, n, W′, and U are as defined herein, and wherein W″ is selected among hydrogen, halogen, hydroxy, thiol, cyano, C₁₋₆alkyl, C₁₋₆haloalkyl, C₁₋₆alkoxy, C₁₋₆alkylsulfenyl, C₁₋₆alkylsulfinyl, C₁₋₆ alkylsulfonyl.

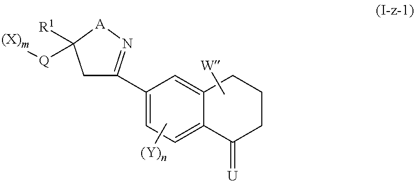

Embodiment Z1: Compounds as defined in embodiment Z, wherein the group Q is selected among Q-1, Q-2, Q-42 to Q-54 and X is independently selected among hydrogen, halogen, amino, C₁₋₆ alkylamino, C₂₋₁₂ dialkylamino, nitro, C₁₋₆alkoxy, and C₁₋₆haloalkyl.

Embodiment Z2: Compounds as defined in embodiment Z or Z1, wherein A represents oxygen or CH₂, R¹ represents CF₃, and Y is H and/or U stands for O, hydroxy-N, C₁₋₆ alkoxy-N, C₁₋₆ haloalkoxy-N.

As used herein, the term “alkyl” refers to linear or branched C₁₋₁₂ alkyl including, for example, ethyl, methyl, n- or iso-propyl, n-, iso-, sec-, or tert-butyl, n-pentyl, n-hexyl, n-heptyl, n-octyl, n-nonyl, n-decyl, n-undecyl and n-dodecyl, and preferably refers to C₁₋₆ alkyl. The alkyl moiety in a group having alkyl as a part of its formula may have the same meaning as described for the aforementioned “alkyl”. The alkyl group can be unsubstituted or substituted with at least one suitable substituent.

The term “halogen” and a halogen moiety in a group substituted with halogen represents fluorine, chlorine, bromine and iodine. Preferred halogens are fluorine, chlorine and bromine.

The term “haloalkyl” used alone or combined with other terms refers to alkyl groups which are partially or fully substituted with halogen atoms which may be the same or different. Examples of “haloalkyl” includes for example CF₃, CH₂F, CHF₂, CCl₃, CH₂Cl, CHCl₂, CF₂CF₃, CHFCF₃, The haloalkyl group can additionally be unsubstituted or substituted with at least one suitable substituent.

The term “cycloalkyl” refers to C₃₋₈ cycloalkyl including, for example, cyclopropyl, cyclobutyl, cyclopentyl, cyclohexyl, cycloheptyl and cyclooctyl, and preferably refers to C₃₋₇ cycloalkyl. The cycloalkyl group can be unsubstituted or substituted with at least one suitable substituent.

The term “alkenyl” refers to C₂₋₅ alkenyl including, for example, vinyl, allyl, 1-propenyl, 1-(or 2- or 3-) butenyl and 1-pentenyl, and preferably refers to C₂₋₄ alkenyl. The alkenyl group can be unsubstituted or substituted with at least one suitable substituent.

The term “alkynyl” used either alone or combined with other terms preferably stands for alkynyl having 2 to 6 or 2 to 5 carbon atoms. Examples include ethynyl, propargyl, 1-propynyl, but-3-ynyl or pent-4-ynyl. More preferred it stands for alkynyl having 2 to 4 carbon atoms. The alkinyl group can be unsubstituted or substituted with at least one suitable substituent.

A “heterocyclic group” preferably refers to a 5- or 6-membered heterocyclic group containing at least one of hetero atoms selected from N, O or S, and said heterocyclic group also refers to a condensed heterocyclic group which may be benzo-condensed. Typically a heterocyclic group contains no more than 4 nitrogens, 2 oxygens and 2 sulfur atoms. The cyclic group can be saturated, unsaturated or partially saturated. If not mentioned otherwise, then a heterocyclic group can be can be attached through any available carbon or heteroatom. Heterocyclic group include for example furyl, thienyl, pyrrolyl, isoxazolyl, pyrazolyl, oxazolyl, oxathiazolyl, imidazolyl, triazolyl, oxadiazolyl, thiadiazolyl, tetrazolyl, pyridyl, pyrimidinyl, pyridazinyl, pyrazinyl, triazinyl, indolyl, benzoxazolyl or quinolyl. The heterocyclic group can be unsubstituted or substituted with at least one suitable substituent.

The term “acylamino” refers to, for example, alkyl-carbonylamino, cycloalkyl-carbonylamino and benzoylamino, wherein the alkyl moiety may the same meaning as described for the aforementioned “alkyl”, and cycloalkyl moiety may have the same meaning as described below. The acylamino group can be unsubstituted or substituted with at least one suitable substituent.

Suitable substituents include for example the following chemical groups, namely amino, hydroxy, halogen, nitro, cyano, isocyano, mercapto, isothiocyanato, carboxy, carbonamide, SF5, amino-sulfonyl, alkyl, cycloalkyl, alkenyl, cycloalkenyl, alkinyl, monoalkyl-amino, dialkyl-amino, N-alkanoyl-amino, alkoxy, alkenyloxy, alkinyloxy, cycloalkoxy, cycloalkenyloxy, alkoxy-carbonyl, alkenyloxy-carbonyl, alkinyloxy-carbonyl, aryloxycarbonyl, alkanoyl, alkenyl-carbonyl, alkinyl-carbonyl, aryl-carbonyl, alkylthio, cycloalkylthio, alkenylthio, cycloalkenylthio, alkinylthio, alkylsulfenyl, alkylsulfinyl, including both enantiomeric forms of alkylsulfinyl, alkyl-sulfonyl, monoalkyl-aminosulfonyl, dialkyl-aminosulfonyl, alkylphosphinyl, alkylphosphonyl, including both enantiomeric forms of alkylphosphinyl and alkylphosphonyl, respectively, N-alkyl-aminocarbonyl, N,N-dialkyl-aminocarbonyl, N-alkanoyl-amino-carbonyl, N-alkanoyl-N-alkyl-aminocarbonyl, aryl, aryloxy, benzyl, benzyloxy, benzylthio, arylthio, arylamino, benzylamino, heterocyclyl and trialkylsilyl. Substituents which are further substituted, like for example alkoxyalkyl, alkylthioalkyl, alkylthioalkoxy, alkoxyalkoxy, phenethyl, benzyloxy, haloalkyl, haloalkoxy, haloalkylthio, haloalkanoyl, haloalkylcarbonyl, haloalkoxycarbonyl, haloalkoxyalkoxy, haloalkoxyalkylthio, haloalkoxyalkanoyl, haloalkoxyalkyl are also included. Preferred substituents are chloro, fluoro, bromo, iodo, NH₂, NO₂, CN, OH, SH and COOH.

Compounds according to the invention may be prepared by using generally known methods or by combining known methods with the preparation methods described herein.

For example, preparation method (a-1) and (a-2) can be carried out according to the method described in WO2007/021308.

Preparation Method (a-1)

Compounds of formula (I-i-1) as defined in embodiment I, wherein U represents oxygen and W′ stands for a group T¹ can be prepared by preparation methods (a-1), namely by reacting a compound of the formula (II-a)

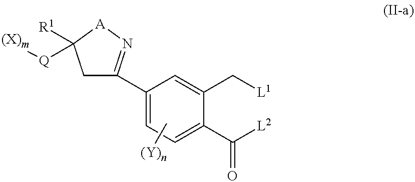

wherein L¹ stands for halogen, alkylsulfonyloxy (e.g. methanesulfonyloxy), or arylsulfonyloxy (e.g. para-toluenesulfonyloxy), and L² stands for alkoxy (e.g. methoxy) or aryloxy (e.g. phenoxy), and wherein (X)ₘ, (Y)ₙ, A, R¹ and Q have the same meaning as described herein, particulary as described for embodiment I, I1 or I2, with a compound of the formula (III):

L³-T¹ (III)

wherein L³ stands for hydroxy, thiol or amino, and T¹ stands for either R³ or R⁴,
if appriopriate, in the presence of a diluent and/or a base to yield compounds of formula (I-i-1) as defined in embodiment I, wherein U represents oxygen and W′ stands for a group T¹.

In carrying out preparation method (a-1), 1 mole of a compound of formula (II-a) can be reacted with 1 to 2 moles of a compound of formula (III) in a diluent, such as toluene, and in the presence of a base, such as potassium carbonate, to obtain the desired compound.

Known compounds of formula (III) include for example water, hydrogen sulfide, ammonia, methylamine, ethylamine, benzylamine, 2-pyridinomethylamine, acetamide. Compounds of formula (I-i-2) as defined in embodiment I, wherein U represents oxygen and W′ stands for a group T¹ canalso be prepared by preparation method (a-1), namely by reacting a compound of the formula (II-b)

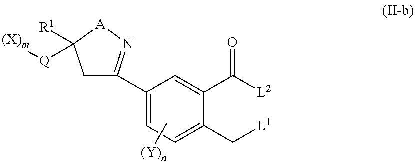

L¹ stands for halogen, alkylsulfonyloxy or arylsulfonyloxy, and L² stands for alkoxy (e.g. methoxy) or aryloxy (e.g. phenoxy), and wherein (X)ₘ, (Y)ₙ, A, R¹ and Q have the same meaning as described herein, particulary as described for embodiment I, I1 or I2, with a compound of formula (III).

Preparation Method (a-2)

Compounds of formula (I-l-1) as defined in embodiment L, wherein U represents oxygen and the two W′ stand for a group T¹ and H, can be prepared by preparation method (a-2), namely by reacting a compound of the formula (II-a) wherein L¹ stands for halogen, alkylsulfonyloxy or arylsulfonyloxy, and L² stands for alkoxy or aryloxy, and wherein (X)ₘ, (Y)ₙ, A, R¹ and Q have the same meaning as described herein, particulary as described for embodiment L, L1 or L2, with a compound of the formula (IV):

H₂NHN-T¹ (IV)

wherein T¹ stand for either R³ or R⁴, and include, among other, methylhydrazine or ethylhydrazine, if appriopriate, in the presence of a diluent and/or a base to yield compounds of formula (I-l-1), wherein U represents oxygen and the one of the two W′ stands for a group T¹ and the other W′ stands for H.

In carrying out Preparation method (a-2), 1 mole of a compound of formula (II-a) can be reacted with 1 to 2 moles of a compound of formula (IV) in a diluent, such as toluene, and in the presence of a base, such as potassium carbonate to obtain the desired compound.

### Preparation Method (b)

Compounds of formula (I-i-1) or (I-i-2) as defined in embodiment I, wherein U represents oxygen and the W′ stands for H can be prepared by preparation method (b), namely by reacting a compound of the formula (V-a)

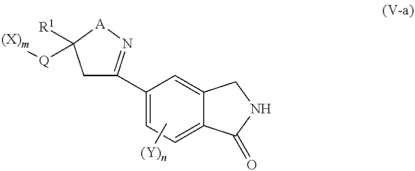

wherein (X)ₘ, (Y)ₙ, A, R¹ and Q have the same meaning as described herein, particulary as described for embodiment I, I1 or I2, with
(i) a compound of the formula (VI-a):

L⁴-T¹ (VI-a)

wherein L⁴ stands for halogen, alkylsulfonyloxy, arylsulfonyloxy or alkylcarbonyloxy, and T¹ stand for either R³ or R⁴; or with
(ii) a compound of the formula (VI-b)

T¹-O-T¹ (VI-b)

wherein T¹ stands for R³ or R⁴; in case T¹ stands for a carbonyl-containing moiety, formula (VI-b) is an acid anhydride;
if appriopriate, in the presence of a diluent and/or a base to yield compounds of formula (I-i-1), wherein U represents oxygen and W′ stands for a group T¹.

Similarly, compounds of formula (I-i-2) can be prepared by reacting a compound of formula (V-b):

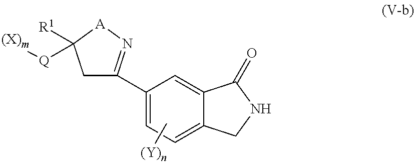

wherein (X)ₘ, (Y)ₙ, A, R¹ and Q have the same meaning as described herein, particulary as described for embodiment I, I1 or I2, with compounds of the formula (VI-a) or (VI-b).

In carrying out preparation method (b), 1 mole of a compound of formula (V-a) or (V-b) can be reacted with 1 to 10 moles of a compound of formula (VI-a) or (VI-b) in a diluent, for example toluene, in the presence of base to obtain the desired compound.

Compounds of formula (VI-a) and (VI-b) include, for example, acetic acid anhydride, acetyl chloride, methyl iodide, and benzyl bromide.

Similar preparation methods are described in WO99/05055, which methods are hereby incorporated.

### Preparation Method (c)

Compounds of formula (I-h-1) as defined in embodiment H, wherein U represents N-T¹ can be prepared by preparation method (c), namely by

reacting a compound of the formula (VII-a)

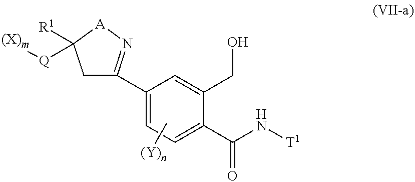

wherein (X)ₘ, (Y)ₙ, A, R¹ and Q have the same meaning as described herein, particulary as described for embodiment H, H1 or H2, and wherein T¹ stands for either R³ or R⁴, with alkyl-sulfonyl chloride or phenylsulfonyl chloride, if appropriate, in the presence of a base, and/or in the presence of a diluent.

Compounds of formula (I-h-2) as defined in embodiment H, wherein U represents N-T¹, wherein T¹ is as defined herein, can be prepared by preparation method (c) by using compounds of the formula (VII-b) as a starting material

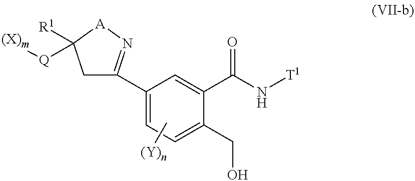

wherein (X)ₘ, (Y)ₙ, A, R¹ and Q have the same meaning as described herein, particulary as described for embodiment H, H1 or H2, and wherein T¹ stands for either R³ or R⁴.

Preparation method (c) can be carried out according to the method described in Synlett (2006), 801-803 which is hereby incorporated by reference.

In carrying out Preparation method (c), 1 mole of a compound of formula (VII-a) can be reacted with 1 to 2 moles of methane sulfonyl chloride in a diluent, for example tetrahydrofuran, in the presence of a base to obtain the desired compound.

### Preparation Method (d)

Compounds of formula (I-i-1) as defined in embodiment I, wherein U represents N—H and W′ stands for T¹ can be prepared by preparation method (d), namely by reacting a compound of formula (VIII-a)

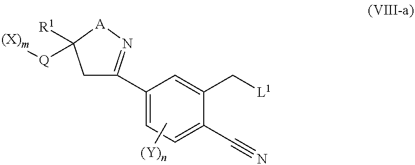

wherein (X)ₘ, (Y)ₙ, A, R¹ and Q have the same meaning as described herein, particulary as described for embodiment I, I1 or I2,
with a compound of formula (IX)

H₂N-T¹ (IX)

wherein T¹ stands for either R³ or R⁴, if appropriate, in the presence of a base, and/or in the presence of a diluent.

Compounds of formula (I-i-2) as defined in embodiment I, wherein U represents N—H and W′ stands for T¹ can be prepared by preparation method (d), using a compound of formula (VIII-b)

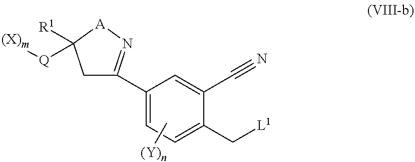

wherein (X)ₘ, (Y)ₙ, A, R¹, Q and L¹ have the same meaning as described herein as starting material.

Preparation method (d) can be carried out according to the methods described in U.S. Pat. No. 6,376,530, and which preparation methods are hereby incorporated by reference.

In carrying out preparation method (d), 1 to 2 moles of a compound of formula (VIII-a) can be reacted with 1-2 moles of a compound of formula (IX) in a diluent, for example acetonitrile, in the presence of potassium carbonate to obtain the desired compound.

### Preparation Method (e)

Compounds of formula (I-i-1) as defined in embodiment I, wherein U represents N-T¹ and W′ stands for T¹ can be prepared by preparation method (e) namely by reacting a compound of the formula (X-a)

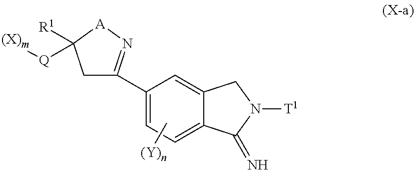

wherein (X)ₘ, (Y)ₙ, A, R¹ and Q have the same meaning as described herein, particulary as described for embodiment I, I1 or I2, with a compound of formula (VI-a) or (VI-b), if appropriate, in the presence of a base, and/or in the presence of a diluent.

Compounds of formula (I-i-2) as defined in embodiment I, wherein U represents N-T¹ and W′ stands for T¹ can be prepared by preparation method (e) using compounds of the formula (X-b):

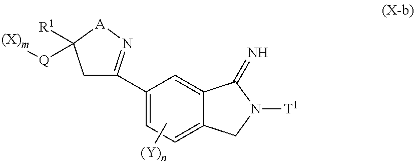

wherein (X)ₘ, (Y)ₙ, A, R¹ and Q have the same meaning as described herein.

Compounds of formula (X-a) or (X-b) can be synthesized according to preparation method (d).

Compounds of formula (VI-a) or (VI-b) include for example acetic acid anhydride, acetyl chloride, methyl iodide, and benzyl bromide.

Preparation method (e) is a known organic synthetic reaction. In the presence of an appropriate base a substitution reaction can occur.

In carrying out preparation method (e), 1 mole of a compound of formula (X) can be reacted with 1 to 1.5 moles of a compound of formula (VI) in a diluent, for example tetrahydrofuran, in the presence of pyridine to obtain a desired compound.

### Preparation Method (f)

Compounds of formula (I-j-1) as defined in embodiment J, wherein W′ stands for T¹ can be prepared by preparation method (f), namely by reacting a compound of the formula (XI)

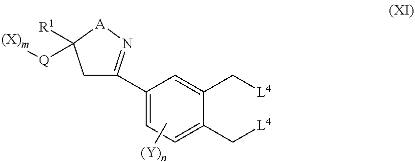

wherein (X)ₘ, (Y)ₙ, A, R¹ and Q have the same meaning as described herein, particulary as described for embodiment J, J1 or J2, with a compound of formula (III), if appropriate, in the presence of a base, and/or in the presence of a diluent.

Compounds aof formula (III) are for example methylamine, ethylamine, benzylamine, 2-pyridinomethylamine or acetamide.

Preparation method (f) can be carried out according to the methods disclosed in Tetrahedron Lett., 2005, 5927-5930 which are hereby incorporated by reference.

In carrying out preparation method (f), 1 mole of a compound of formula (XI) can be reacted with 1 to 2 moles of a compound of formula (III) in a diluent, for example tetrahydrofuran, in the presence of 1 to 2 moles of sodium hydride as the base, to obtain the desired product.

### Preparation Method (g)

Compounds according to the invention wherein A is oxygen can be prepared according to preparation methods (g) and (h), namely by

reacting a compound of the formula (XII)

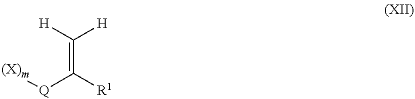

wherein (X)ₘ, R¹ and Q have the same meaning as described herein, particularly as described in the embodiments A, B, C or L, L1 or L2,
with a compound of the following formula (XIII):

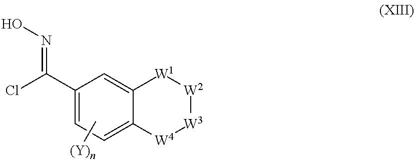

wherein W¹ to W⁴ and (Y)ₙ have the same meaning as described herein, in the presence of inert diluents, and, if appropriate in the presence of a base.

Preparation method (g) can be carried out according to the methods disclosed in WO2004/018410, WO2005/085216, Tetrahedron, 2000, Vol 56, 1057-1064.

In carrying out preparation method (g), 1 mole of a compound of formula (XIII) can be reacted with 1 to 2 moles of a compound of formula (XII) and with 1 mole to a slightly excessive amount of base in a diluent, for example DMF, to obtain the desired compound.

### Preparation Method (h)

Compounds according to the invention wherein A is nitrogen or oxygen can be prepared according to preparation method (h), namely by

reacting a compound of the formula (XIV)

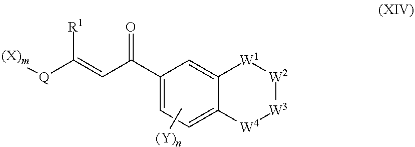

wherein (X)ₘ, (Y)ₙ, A, R¹ and Q have the same meaning as described herein, particularly as described in the embodiments A, B, C, H1, H2, and H3, and wherein W¹ to W⁴ and (Y)ₙ have the same meaning as described herein, with
(i) a compound of the formula (XV):

H₂NHN—R² (XV)

or a hydrochloride acid salt thereof, wherein R² has the same meaning as described herein, to form a pyrazoline ring at the appropriate position; or
(ii) a hydroxyamine or a hydrochloride acid salt thereof to form an isoxazoline ring at the appropriate position;
if appropriate in the presence of a base and optionally in the presence of a diluent.

Examples of the compound of formula (XV) include hydrazine, methylhydrazine and ethylhydrazine.

Compounds according to the invention wherein A is CH₂ can be prepared according to preparation methods (i) and (j).

### Preparation Method (i)

Shifting an imino double bond in compounds of formula (XVI):

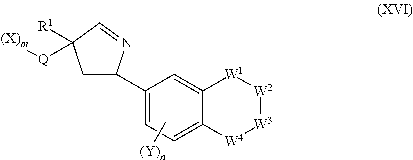

wherein (X)ₘ, A, R¹ and Q have the same meaning as described herein, particularly as described in the embodiments A, B, C, H1, H2, and H3, and wherein W¹ to W⁴ and (Y)ₙ have the same meaning as described herein,
if appropriate, in the presence of a base, and optionally in the presence of a diluent.

Preparation method (i) can be carried out according to the methods disclosed in Japanese Patent Application Laid-Open 2007-91708 and Chem. Lett., 1985, 1601-1604 which are hereby incorporated by reference.

In carrying out preparation method (i), 1 mole to a slightly excessive amount of base can be reacted with 1 mole of a compound of formula (XVI) in a diluent, for example tetrahydrofuran, to obtain a desired compound of formula (I).

### Preparation Method (j)

Reacting a compound of formula (XII) as defined above with a compound of the following formula (XXX):

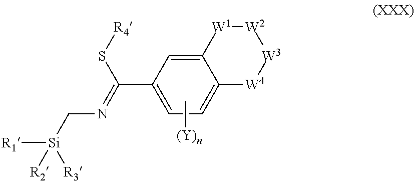

wherein R₁′, R₂′ and R₃′ each independently represent C₁₋₁₂ alkyl, or phenyl; R₄′ represents hydrogen; or is selected among C₁₋₁₂ alkyl, C₁₋₁₂ alkenyl, C₁₋₁₂ alkynyl and benzyl; and
W¹ to W⁴ and (Y)ₙ have the same meaning as described herein, if appropriate, in the presence of a fluoride reagent, such as potassium fluoride, tetramethylammonium fluoride, tetraethylammonium fluoride, or tetrabutylammonium fluoride.

For carrying out preparation method (j) 1 mole of a compound of formula (XII) relative to 1 mole of compound of formula (XXX) can be reacted with 0.1 mole of a fluoride reagent in a diluent, for example THF, to obtain a desired compound of formula (I).

Preparation method (j) can be carried out according to the methods described in J. Org. Chem., Vol. 52, 1027-1035, 1987 which is hereby incorporated by reference.

The preparation methods can be illustrated by the following reaction schemes, by way of examples.

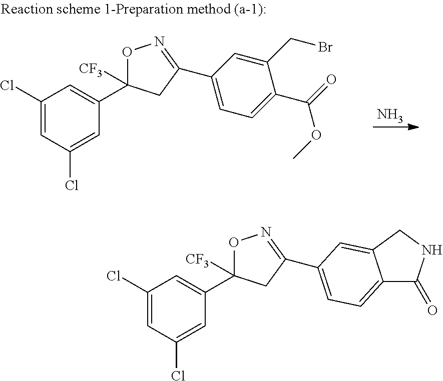

Here, methyl 2-(bromomethyl)-4-[5-(3,5-dichlorophenyl)-5-(trifluoromethyl)-4,5-dihydro-isoxazol-3-yl]benzoate and ammonia are used as starting material.

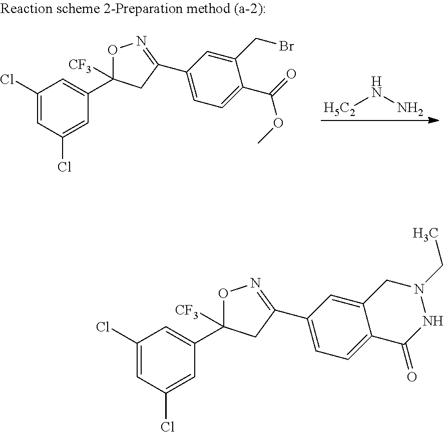

Here, methyl 2-(bromomethyl)-4-[5-(3,5-dichlorophenyl)-5-(trifluoromethyl)-4,5-dihydro-isoxazol-3-yl]benzoate and ethyl hydrazine are used as starting material.

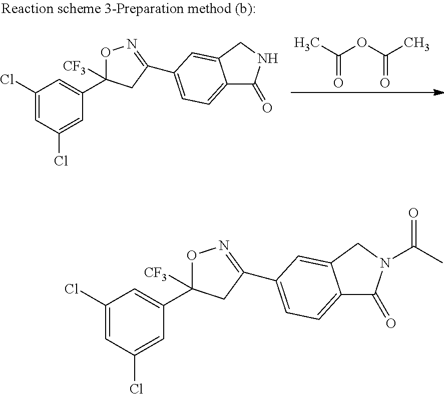

Here, 5-[5-(3,5-dichlorophenyl)-5-(trifluoromethyl)-4,5-dihydro-isoxazol-3-yl]-2,3-dihydro-1H-isoindol-1-one and an acetic anhydride are used as starting material.

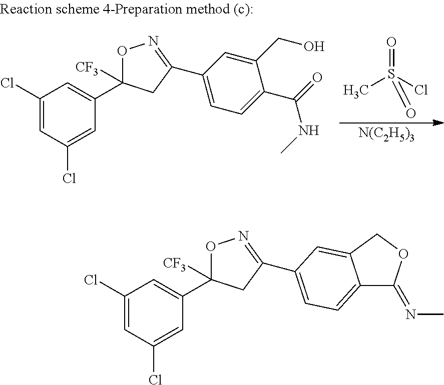

Here, 4-[5-(3,5-dichlorophenyl)-5-(trifluoromethyl)-4,5-dihydroisoxazol-3-yl]-2-(hydroxymethyl)-N-methylbenzamide, methanesulfonyl chloride and triethylamine are used as starting material.

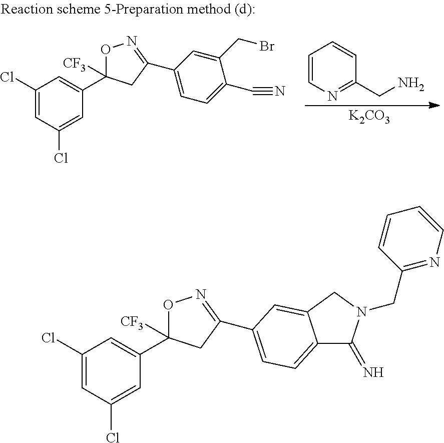

Here, 2-(bromomethyl)-4-[5-(3,5-dichlorophenyl)-5-(trifluoromethyl)-4,5-dihydro-isoxazol-3-yl]benzonitrile and 1-(pyridin-2-yl)methaneamine are used sarting material.

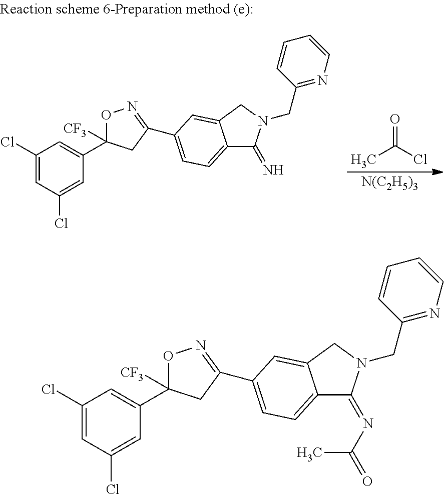

Here, 5-[5-(3,5-dichlorophenyl)-5-(trifluoromethyl)-4,5-dihydro-isoxazol-3-yl]-2-(pyridin-2-ylmethyl)-2,3-dihydro-1H-isoindol-1-imine, acetyl chloride and tiretylamine are used as starting material.

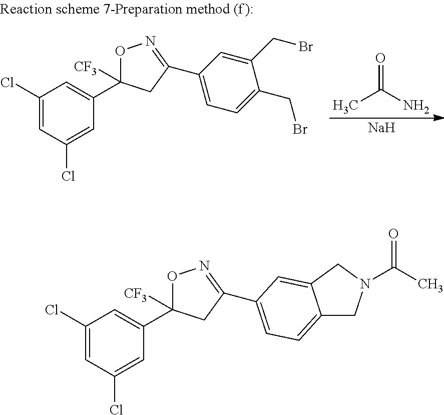

Here, 3-[3,4-bis(bromomethyl)phenyl]-5-(3,5-dichlorophenyl)-5-(trifluoromethyl)-4,5-dihydro-isoxazole, acetamide and sodium hydride are used as starting material.

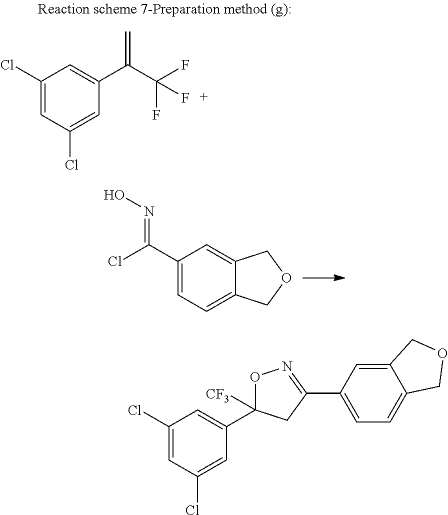

Here, 1,3-dichloro-5-(3,3,3-trifluoroprop-1-en-2-yl)benzene and N-hydroxy-1,3-dihydro-2-benzofuran-5-carboxylmidylchloride are used as starting material.

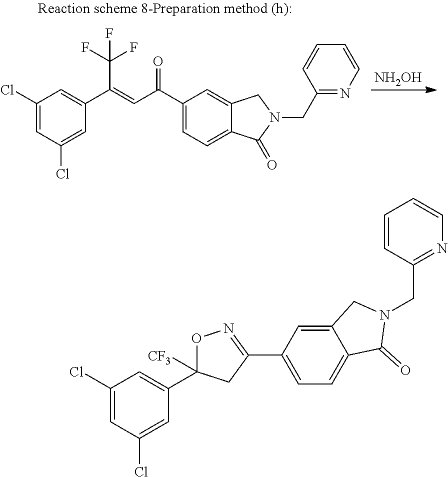

Here, 5-[3-(3,5-dichlorophenyl)-4,4,4-trifluorobut-2-enoyl]-2-(pyridin-2-ylmethyl)-2,3-dihydro-1H-isoindol-1-one and hydroxyamine are used as starting material.

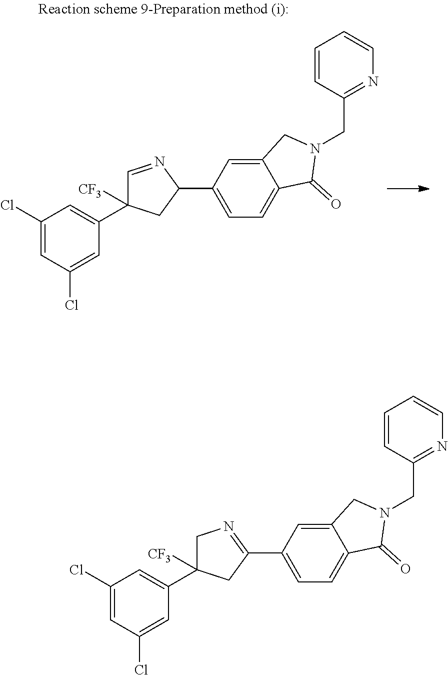

Here, 5-[4-(3,5-dichlorophenyl)-4-(trifluoromethyl)-3,4-dihydro-2H-pyrrol-2-yl]-2-(pyridin-2-ylmethyl)-2,3-dihydro-1H-isoindol-1-one is used as starting material.

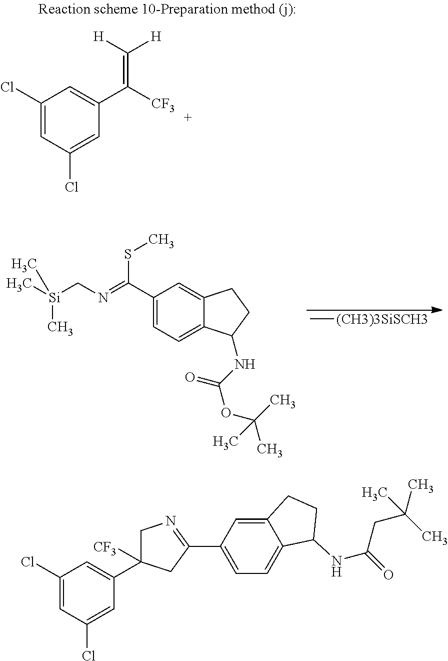

Here, 1,3-dichloro-5-(3,3,3-trifluoroprop-1-en-2-yl)benzene and methyl 1-[(tert-butoxycarbonyl-) amino]-N-[(trimethylsilyl)methyl]-2,3-dihydro-1H-inden-5-carboimide thioate are used as starting material.

Known compounds of formulae (II-a) and (II-b) can be obtained by reacting compounds of formula (XVII-a) or (XVII-b)

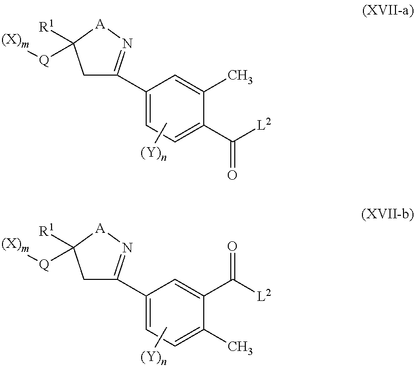

wherein (X)ₘ, (Y)ₙ, A, R¹, Q and L² have the same meaning as described herein, with a halogenating agent, such as chlorine, bromine, iodine, N-chlorosuccinimide, N-bromosuccinimide, N-iodosuccinimide, 1,3-dichloro-5,5-hydantoin, 1,3-dibromo-5,5-dimethylhydantoin, benzyl trimethyl ammonium tetrachloroiodate and sodium hypochlorite, if appropriate, in the presence of a catalyst (e.g. AIBN (2,2′-azabisisobutyronitrile) or benzoyl peroxide).

Compounds of formulae (II-a) or (II-b) are for example methyl 2-(bromomethyl)-4-[5-(3,5-dichlorophenyl)-5-(trifluoromethyl)-4,5-dihydroisoxazol-3-yl]benzoate, methyl 2-(bromomethyl)-5-[5-(3,5-dichlorophenyl)-5-(trifluoromethyl)-4,5-dihydroisoxazol-3-yl]benzoate, methyl 2-(bromomethyl)-4-[5-(3,5-dichlorophenyl)-1-methyl-5-(trifluoromethyl)-4,5-di-hydro-1H-pyrazol-3-yl]benzoate, and methyl 2-(bromomethyl)-5-[5-(3,5-dichlorophenyl)-1-methyl-5-(trifluoromethyl)-4,5-dihydro-1H-pyrazol-3-yl]benzoate.

Compounds of formula (V-a) or (V-b) can be obtained through preparation method (a-1) and include 5-[5-(3,5-dichlorophenyl)-5-(trifluoromethyl)-4,5-dihydroisoxazol-3-yl]-2,3-dihydro-1H-isoindol-1-one, 6-[5-(3,5-dichlorophenyl)-5-(trifluoromethyl)-4,5-dihydro-isoxazol-3-yl]-2,3-dihydro-1H-isoindol-1-one, 5-[5-(3,5-dichlorophenyl)-1-methyl-5-(trifluoromethyl)-4,5-dihydro-1H-pyrazol-3-yl]-2,3 dihydro-1H-isoindol-1-one, and 6-[5-(3,5-dichlorophenyl)-1-methyl-5-(trifluoromethyl)-4,5-dihydro-1H-pyrazol-3-yl]-2,3-dihydro-1H-isoindol-1-one.

Compounds of formula (VII-a) or (VII-b) include for example 4-[5-(3,5-dichlorophenyl)-5-(trifluoromethyl)-4,5-dihydroisoxazol-3-yl]-2-(hydroxymethyl)-N-methylbenzamide, 5-[5-(3,5-dichlorophenyl)-5-(trifluoromethyl)-4,5-dihydroisoxazol-3-yl]-2-(hydroxymethyl)-N-methylbenzamide, 4-[5-(3,5-dichlorophenyl)-1-methyl-5-(trifluoromethyl)-4,5-dihydro-1H-pyrazol-3-yl]-2-(hydroxymethyl)-N-methylbenzamide, and 5-[5-(3,5-dichloro-phenyl)-1-methyl-5-(trifluoromethyl)-4,5-dihydro-1H-pyrazol-3-yl]-2-(hydroxymethyl)-N-methyl-benzamide.

Compounds of formula (VII-a) or (VII-b) can be obtained by reacting a compound of formula (XIX-a) or (XIX-b), which can be obtained by using preparation method (a),

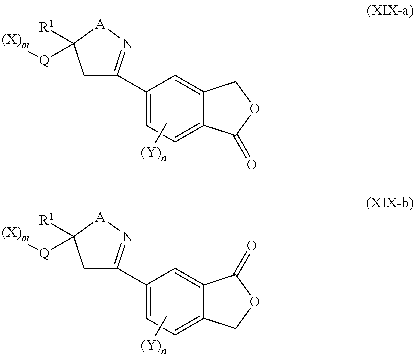

wherein (X)ₘ, (Y)ₙ, A, R¹ and Q have the same meaning as described herein, with a compound of formula (IX), if appropriate, in the presence of a base.

Compounds of formula (XIX-a) and (XIX-b) can be obtained according to preparation method (a) and according to the method disclosed in EP-A-1 362 856. For example by reacting compounds of formula (II-a) or (II-b) with sodium acetate to give the compounds of the formula (XX-a) and (XX-b), respectively

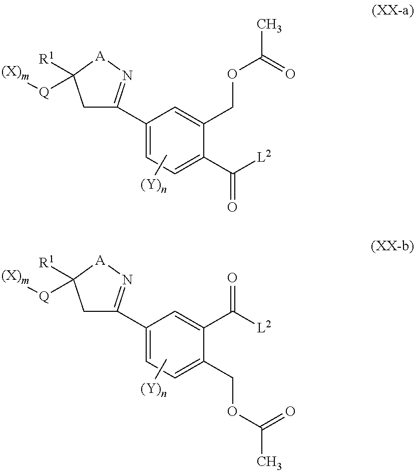

wherein (X)ₘ, (Y)ₙ, A, R¹, Q and L² have the same meaning as described herein, following reation with a base in alcohol.

Compounds of formula (XIX-a) or (XIX-b) include for example 5-[5-(3,5-dichlorophenyl)-5-(trifluoromethyl)-4,5-dihydroisoxazol-3-yl]-2-benzofuran-1(3H)-one, 6-[5-(3,5-dichlorophenyl)-5-(trifluoromethyl)-4,5-dihydroisoxazol-3-yl]-2-benzofuran-1(3H)-one, 5-[5-(3,5-dichlorophenyl)-5-(trifluoromethyl)-4,5-dihydroisoxazol-3-yl]-2-benzofuran-1(3H)-one, 6-[5-(3,5-dichlorophenyl)-5-(trifluoromethyl)-4,5-dihydroisoxazol-3-yl]-2-benzofuran-1(3H)-one.

Compounds of formula (ω-a) and (XX-b) include for example Methyl 2-[(acetyloxy)methyl]-4-[5-(3,5-dichlorophenyl)-5-(trifluoromethyl)-4,5-dihydro-isoxazol-3-yl]benzoate, Methyl 2-[(acetyloxy)methyl]-5-[5-(3,5-dichlorophenyl)-5-(trifluoromethyl)-4,5-dihydroisoxazol-3-yl]benzoate, Methyl 2-[(acetyloxy)methyl]-4-[5-(3,5-dichlorophenyl)-1-methyl-5-(trifluoromethyl)-4,5-dihydro-1H-pyrazol-3-yl]benzoate, Methyl 2-[(acetyloxy)methyl]-5-[5-(3,5-dichlorophenyl)-1-methyl-5-(trifluoromethyl)-4,5-dihydro-1H-pyrazol-3-yl]benzoate.

Compounds of formula (VIII-a) or (VIII-b) can be obtained according to the preparation method described herein for the preparation of compounds of formula (II-a).

Compounds of formula (VIII-a) or (VIII-b) include for example 2-(bromomethyl)-4-[5-(3,5-dichlorophenyl)-5-(trifluoromethyl)-4,5-dihydroisoxazol-3-yl]benzonitrile, 2-(bromomethyl)-5-[5-(3,5-dichlorophenyl)-5-(trifluoromethyl)-4,5-dihydro-soxazol-3-yl]benzonitrile, 2-(bromomethyl)-4-[5-(3,5-dichlorophenyl)-1-methyl-5-(trifluoromethyl)-4,5-dihydro-1H-pyrazol-3-yl]benzonitrile, and 2-(bromomethyl)-5-[5-(3,5-dichlorophenyl)-1-methyl-5-(trifluoromethyl)-4,5-dihydro-1H-pyrazol-3-yl]benzonitrile.

Compounds of formula (IX) include for example methylamine, ethylamine, benzylamine, 2-pyridinomethylamine, and acetamide.

Compounds of formula (X-a) or (X-b) include for example 5-[5-(3,5-dichlorophenyl)-5-(trifluoromethyl)-4,5-dihydroisoxazol-3-yl]-2-(pyridin-2-ylmethyl)-2,3-dihydro-1H-isoindol-1-imine,
6-[5-(3,5-dichlorophenyl)-5-(trifluoromethyl)-4,5-dihydroisoxazol-3-yl]-2-(pyridin-2-ylmethyl)-2,3-dihydro-1H-isoindol-1-imine,
5-[5-(3,5-dichlorophenyl)-1-methyl-5-(trifluoromethyl)-4,5-dihydro-1H-pyrazol-3-yl]-2-(pyridin-2-ylmethyl)-2,3-dihydro-1H-isoindol-1-imine, and
6-[5-(3,5-dichlorophenyl)-1-methyl-5-(trifluoromethyl)-4,5-dihydro-1H-pyrazol-3-yl]-2-(pyridin-2-ylmethyl)-2,3-dihydro-1H-isoindol-1-imine.

Compounds of formula (XVII-a) and (XVII-b) can be synthesized according to known methods (cf. WO2004/018410, WO2005/085216, Tetrahedron, 2000, Vol 56, 1057-1064, or WO2007/074789) and/or via preparation method (h) by using the compounds of formula (XVIII-a) or (XVIII-b) as starting material

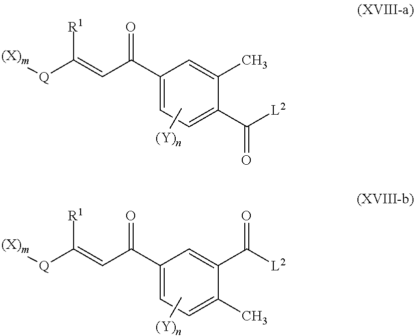

wherein (X)ₘ, (Y)ₙ, R¹ and Q have the same meaning as described herein, and reacting these compounds with a compound of formula (XV) or the hydrochloride acid salt therefrom, or, alternatively, with hydroxyamine or the hydrochloride acid salt therefrom, if appropriate, in the presence of a base.

Compounds of formula (XVII-a) or (XVII-b) are for example methyl 2-methyl-4-[5-(3,5-dichloro-phenyl)-5-(trifluoromethyl)-4,5-dihydroisoxazol-3-yl]benzoate, methyl 2-methyl-5-[5-(3,5-dichlorophenyl)-5-(trifluoromethyl)-4,5-dihydroisoxazol-3-yl]benzoate, methyl 2-methyl-4-[5-(3,5-dichlorophenyl)-1-methyl-5-(trifluoromethyl)-4,5-dihydro-1H-pyrazol-3-yl]benzoate, and methyl 2-methyl-5-[5-(3,5-dichlorophenyl)-1-methyl-5-(trifluoromethyl)-4,5-dihydro-1H-pyrazol-3-yl]benzoate.

Compounds of formula (XI) can be synthesized by diluting a compound of the formula (XXI)

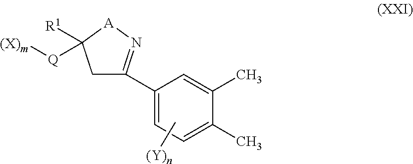

wherein (X)ₘ, (Y)ₙ, A, R¹ and Q have the same meaning as described herein, with an appropriate diluent, for example dichloroethane, and then treating the mixture with a halogenating agent such as N-bromosuccinimide.

Compounds of formula (XI) include for example 3-[3,4-bis(bromomethyl)phenyl]-5-(3,5-dichlorophenyl)-5-(trifluoromethyl)-4,5-dihydroisoxazole, and 3-[3,4-bis(bromomethyl)phenyl]-5-(3,5-dichlorophenyl)-1-methyl-5-(trifluoromethyl)-4,5-dihydro-1H-pyrazole.

Compounds of formula (XXI) can be obtained based on the synthesizing route described for compounds of formula (XVII-a) or (XVII-b). Compounds of formula (XXI) include for example 5(3,5-dichlorophenyl)-3-(3,4-dimethylphenyl)-5-(trifluoromethyl)-4,5-dihydroisoxazole, 5-(3,5-dichlorophenyl)-3-(3,4-dimethylphenyl)-1-methyl-5-(trifluoromethyl)-4,5-dihydro-1H-pyrazole.

Compounds of formula (XII) are also described in The Journal of Organic Chemistry, 1991, Vol 56, 7336-7340, ibid, 1994, Vol 59, 2898-2901, Journal of Fluorine Chemistry, 1999, Vol 95, 167-170, or WO2005/05085216.

Compounds of formula (XII) include for example [1-(trifluoromethyl)vinyl]benzene, 1,3-difluoro-5-[1-(trifluoromethyl)vinyl]benzene, 1-chloro-3-[1-(trifluoromethyl)vinyl]benzene, 1,3-dichloro-5-[1-(trifluoromethyl)vinyl]benzene, 1-trifluoromethyl-3-[1-(trifluoromethyl)vinyl]benzene, 1-trifluoromethyl-4-[1-(trifluoromethyl)vinyl]benzene, 1,3-bis(trifluoromethyl)-5-[1-(trifluoro-methyl)vinyl]benzene, 1,3-dibromo-5-[1-(trifluoromethyl)vinyl]benzene, and 1,2,3-trichloro-5-[1-(trifluoromethyl)vinyl]benzene, 1-fluoro-2-(trifluoromethyl)-4-[1-(trifluoromethyl)vinyl]benzene.

Compounds of formula (XIII) can be obtained by reacting compounds of formula (XXII)

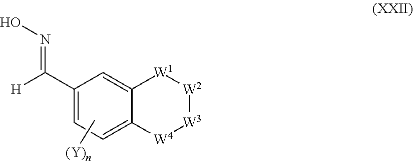

wherein W¹ to W⁴ and (Y), have the same meaning as described herein, with a halogenating agent.

Compounds of formula (XXII) can be obtained by reacting compounds of the formula (XXIII)

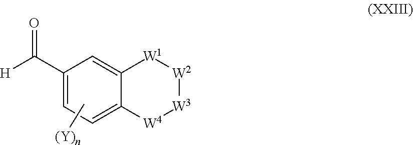

wherein W¹ to W⁴ and (Y)ₙ have the same meaning as described herein, with hydroxyamine or a salt thereof.

Compounds of formula (XXIII) include for example 1H-indol-5-carbaldehyde, t-butyl 5-formyl-1H-indol-1-carboxyate, 1H-indol-6-carbaldehyde, t-butyl 6-formyl-1H-indol-1-carboxyate, 2,3-dihydro-1H-indol-5-carbaldehyde, t-butyl 5-formyl-2,3-dihydro-1H-indol-1-arboxyate, 1-oxo-2,3-dihydro-1H-inden-5-carbaldehyde, 5-oxo-5,6,7,8-tetrahydronaphthalen-2-carbaldehyde, 2,3-dihydro-1-benzofuran-5-carbaldehyde, 1,3-benzdioxol-5-carbaldehyde, 1,4-benzodioxan-6-carbaldehyde, 1-oxo-1,3-dihydro-2-benzo-furan-5-carbaldehyde, and 1-oxo-2-(pyridin-2-ylmethyl)-2,3-dihydro-1H-isoindol-5-carbaldehyde.

### Reaction Scheme 11 and 12—Methods for the Preparation of Compounds of Formula (XXIII):

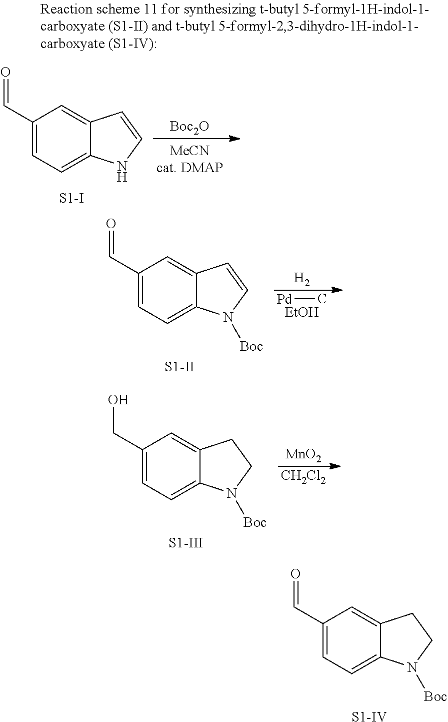

(wherein, Boc represents a t-butoxycarbonyl group, MeCN represents acetonitrile, cat.DMAP represents a catalytic amount of 4-dimethylaminopyridine, Pd—C represents palladium charcoal and ETOH represents ethanol).

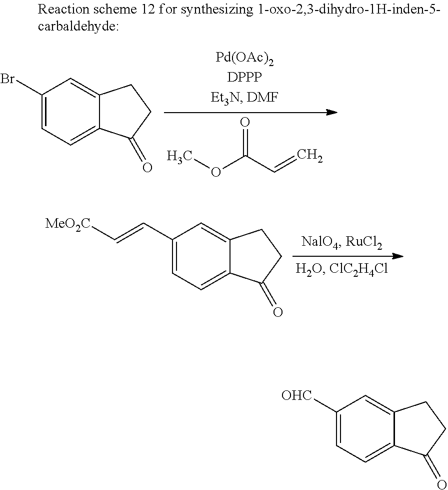

(wherein, Ac represents an acetyl group, DPPP represents 1,3-bis(diphenylphosphino)propane, Et3N represents triethylamine, DMF represents dimethylformamide, and Me represents a methyl group).

5-Oxo-5,6,7,8-tetrahydronaphthalen-2-carbaldehyde can be synthesized in the same manner as the method of Scheme 12, by using 6-bromo-3,4-dihydronaphthalen-1(2H)-one as starting material.

Halogenating agents which are suitable for the preparation of compounds of the formula (XIII) include chlorine, bromine, iodine, N-chlorosuccinimide, N-bromosuccinimide, N-iodosuccinimide, 1,3-dichloro-5,5-hydantoin, 1,3-dibromo-5,5-dimethylhydantoin, benzyl trimethyl ammonium tetrachloroiodate, and sodium hypochlorite.

Compounds of formula (XXIII) include for example N-hydroxy-1H-indol-5-carboxyImidoyl chloride, t-butyl 5-[chloro(hydroxyimino)methyl]-1H-indol-1-carboxyate, t-butyl-5-[chloro-(hydroxyimino)methyl]-2,3-dihydro-1H-indol-1-carboxyate, N-hydroxy-1H-indol-6-carboxy-imidoyl chloride, N-hydroxy-2,3-dihydro-1-benzofuran-5-carboxyImidoyl chloride, N-hydroxy-1,3-benzodioxol-5-carboxyImidoyl chloride, N-hydroxy-2,3-dihydro-1,4-benzo-dioxin-6-carboxyImidoyl chloride, N-hydroxy-1-oxo-1,3-dihydro-2-benzofuran-5-carboxyImidoyl chloride, N-hydroxy-1-oxo-2-(pyridin-2-ylmethyl)-2,3-dihydro-1H-isoindol-5-carboxyImidoyl chloride and N-hydroxy-1-oxo-2,3-dihydro-1H-inden-5-carboxylmidyl chloride.

With respect to preparation method (g), once an isoxazoline ring is constructed, substitution with various substituents can be made. Reaction scheme 13 and Reaction scheme 14 show synthetic method examples to introduce substituents.

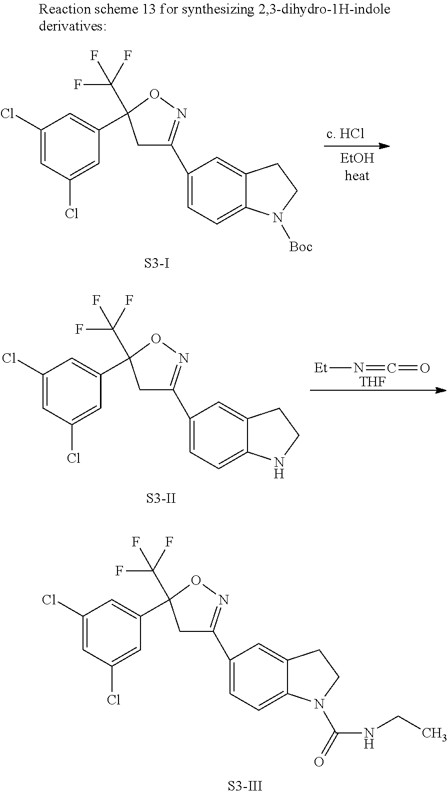

(wherein, c.HCl represents concentrated hydrochloric acid, Et represents an ethyl group, heat represents a heating treatment and THF represents tetrahydrofuran).

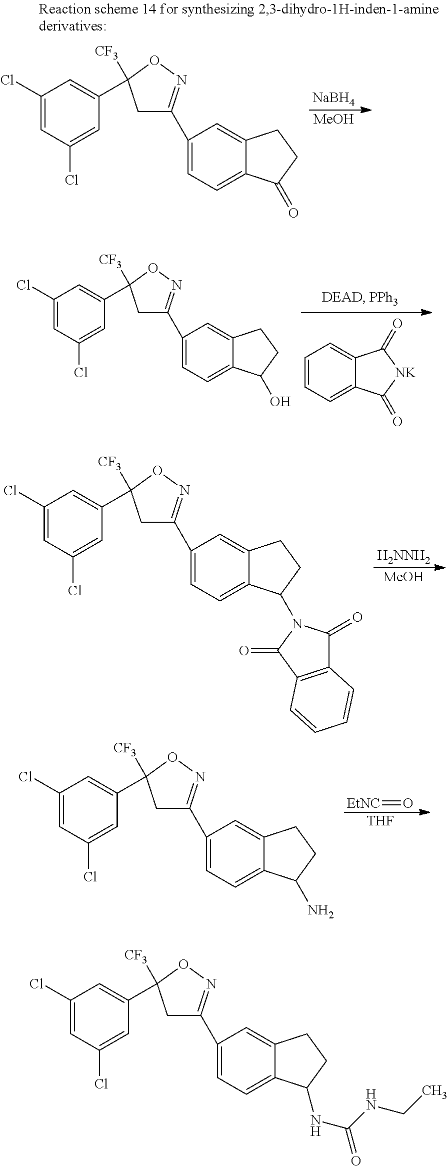

(wherein, Me represents a methyl group, DEAD represents diethyl azodicarboxyate, Ph represents a phenyl group, Et represents an ethyl group, and THF represents tetrahydrofuran).

Preparation method (g) can be applied to the synthetic method comprising reacting the compounds of formula (XXXI) which fall under the general formula (XXXI-a):

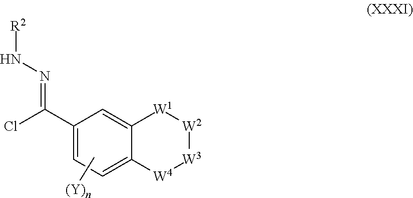

wherein R², W¹ to W⁴ and (Y)ₙ have the same meaning as described herein, with the compounds of formula (XII) described herein.

The invention is further directed to useful intermediate compounds of formula (XXXI-a) for the preparation of compounds according the invention

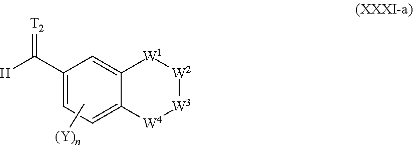

wherein T² stands for O, N—OH and N—NH—R², and W¹ to W⁴ and (Y)ₙ have the same meaning as defined herein.

Compounds of the formula (XXII), (XXIII) and (XXXII), which are useful intermediates for the preparation of compounds according to the invention, are represented by the formula (XXXI-a).

Compounds of formula (XXXI) can be prepared using the same preparation methods as described for compounds of formula (XIII), however, starting with (XXXII).

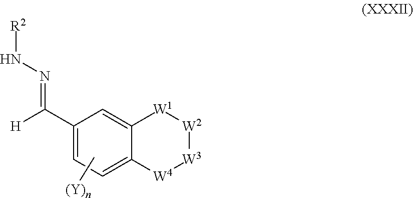

The compounds of formula (XIV), which are useful intermediates for the preparation of compounds according to the invention, can be obtained by reacting compounds of formula (XXIV)

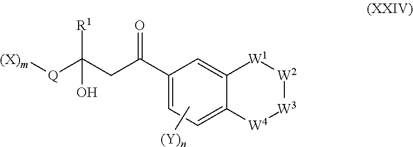

wherein W¹ to W⁴, (X)ₘ, (Y)ₙ, R¹ and Q have the same meaning as described herein with thionyl chloride.

Compounds of formula (XXIV) are for example 3-(3,5-dichlorophenyl)-4,4,4-trifluoro-3-hydroxy-1-(1H-indol-5-yl)butan-1-one, 5-[3-(3,5-dichlorophenyl)-4,4,4-trifluoro-3-hydroxybutanoyl]-2-benzofuran-1(3H)-one, 5-[3-(3,5-dichlorophenyl)-4,4,4-trifluoro-3-hydroxybutanoyl]-2-(pyridin-2-ylmethyl)-2,3-dihydro-1H-isoindol-1-one, 3-(3,4,5-trichloro-phenyl)-4,4,4-trifluoro-3-hydroxy-1-(1H-indol-5-yl)butan-1-one, 5-[3-(3,4,5-trichlorophenyl)-4,4,4-trifluoro-3-hydroxybutanoyl]-2-benzofuran-1(3H)-one, 5-[3-(3,4,5-trichlorophenyl)-4,4,4-trifluoro-3-hydroxybutanoyl]-2-(pyridin-2-ylmethyl)-2,3-dihydro-1H-isoindol-1-one, 3-[3,5-bis(trifluoromethyl)phenyl]-4,4,4-trifluoro-3-hydroxy-1-(1H-indol-5-yl)butan-1-one, 5-{3-[3,5-bis(trifluoromethyl)phenyl]-4,4,4-trifluoro-3-hydroxybutanoyl}-2-benzofuran-1(3H)-one, 5-{3-[3,5-bis(trifluoromethyl)phenyl]-4,4,4-trifluoro-3-hydroxybutanoyl}-2-(pyridin-2-ylmethyl)-2,3-dihydro-1H-isoindol-1-one.

Compounds of formula (XXIV) can be synthesized according to a method disclosed in Zhurnal Organicheskoi Khimii, Vol 28 (No. 3), 518-526, namely by reacting the compound represented by the formula (XXV)

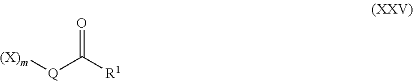

(wherein (X)ₘ, Q and R¹ have the same meaning as described herein) with the compound represented by the following formula (XXVI):

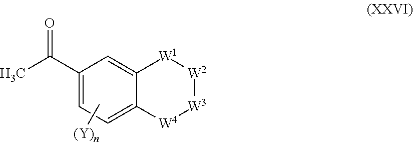

(wherein W¹ to W⁴ and (Y)ₙ have the same meaning as described herein).

Compounds of formula (XXV) include for example trifluoroacetophenone, 3′,5′-dichloro-2,2,2-trifluoroacetophenone, 3′,4′-dichloro-2,2,2-trifluoroacetophenone, 3′,4′,5′-trichloro-2,2,2-trifluoroacetophenone, 3′-fluoro-2,2,2-trifluoroacetophenone, 3′-chloro-2,2,2-trifluoroacetophenone, 3′-bromo-2,2,2-trifluoroacetophenone, 3′-iodo-2,2,2-trifluoroaceto-phenone, 3′-nitro-2,2,2-trifluoroacetophenone, 3′-cyano-2,2,2-trifluoroacetophenone, 3′-(trifluoro-methyl)-2,2,2-trifluoroacetophenone, and 3′,5′-bis(trifluoromethyl)-2,2,2-trifluoroacetophenone.

Compounds of formula (XXVI) include for example 5-acetylindane, 6-acetyltetraline, 3′,4′-(methylenedioxy)acetophenone, 1,4-benzodioxane-6-ylmethyl ketone, 5-acetyl-2,3-dihydro-1-benzofuran, 5-acetyl-1H-indole, 5-acetyl-2-benzofuran-1(3H)-one, and 5-acetyl-2-(pyridin-2-yl-methyl)-2,3-dihydro-1H-isoindol-1-one.

Compounds of formula (XIV) include for example 3-(3,5-dichlorophenyl)-4,4,4-trifluoro-1-(1H-indol-5-yl)but-2-ene-1-one, 5-[3-(3,5-dichlorophenyl)-4,4,4-trifluoro-2-enoyl]-2-benzofuran-1(3H)-one, 5-[3-(3,5-dichlorophenyl)-4,4,4-trifluorobut-2-enoyl]-2-(pyridin-2-ylmethyl)-2,3-dihydro-1H-isoindol-1-one, 3-(3,4,5-trichlorophenyl)-4,4,4-trifluoro-1-(1H-indol-5-yl)but-2-ene-1-one, 5-[3-(3,4,5-trichlorophenyl)-4,4,4-trifluoro-2-enoyl]-2-benzofuran-1(3H)-one, 5-[3-(3,4,5-trichlorophenyl)-4,4,4-trifluorobut-2-enoyl]-2-(pyridin-2-ylmethyl)-2,3-dihydro-1H-isoindol-1-one, 3-[3,5-bis(trifluoromethyl)phenyl]-4,4,4-trifluoro-1-(1H-indol-5-yl)but-2-ene-1-one, 5-{3-[3,5-bis(trifluoromethyl)phenyl]-4,4,4-trifluoro-2-enoyl}-2-benzofuran-1(3H)one, and 5-{3-[3,5-bis(trifluoromethyl)phenyl]-4,4,4-trifluorobut-2-enoyl}-2-(pyridin-2-yl-methyl)-2,3-dihydro-1H-isoindol-1-one.

Preparation method (h) can also be used to prepare compounds of formula (XVII-a) and (XVII-b) from the compounds of formula (XVIII-a) and (XVIII-b).

Compounds of formula (XVI) can be synthesized according to the method disclosed in EP-A-1 538 138, namely by reacting the compound represented by the formula (XXVII):

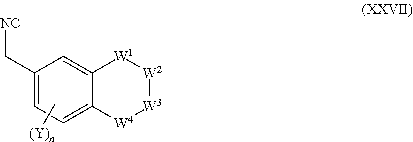

(wherein W¹ to W⁴ and (Y), have the same meaning as described herein).
with the compound of formula (XII), if appropriate, in the presence of a metal catalyst (e.g. copper(II) oxide).

Representative examples of the compound of formula (XVI) include 5-[4-(3,5-dichlorophenyl)-4-(trifluoromethyl)-3,4-dihydro-2H-pyrrol-2-yl]-2-(pyridin-2-ylmethyl)-2,3-dihydro-1H-isoindol-1-one, N-[5-[4-(3,5-dichlorophenyl)-4-(trifluoromethyl)-3,4-dihydro-2H-pyrrol-2-yl]-2-(pyridin-2-ylmethyl)-2,3-dihydro-1H-isoindol-1-yliden]acetamide, and 4-(3,5-dichlorophenyl)-2-(1,3-dihydro-2-benzofuran-5-yl)-4-(trifluoromethyl)-3,4-dihydro-2H-pyrrole.

The compound of formula (XXVII) can be synthesized according to the method disclosed in Chem. Lett., 1977, 697-698, or by reacting compounds of the formula (XXVIII)

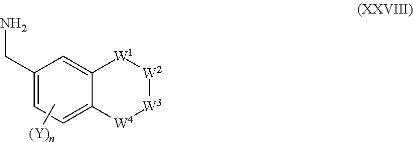

(wherein W¹ to W⁴ and (Y)ₙ have the same meaning as described herein) with ethyl formate, to obtain the compound of the formula (XXIX):

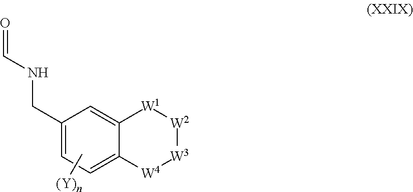

followed by steps of halogenation and dehydrohalogenation.

Compounds of formula (XXVI) include for example 5-(isocyanomethyl)-2-(pyridin-2-ylmethyl)-2,3-dihydro-1H-isoindol-1-one, N-[5-(isocyanomethyl)-2-(pyridin-2-ylmethyl)-2,3-dihydro-1H-isoindol-1-yliden]acetamide, and 1,3-dihydro-2-benzofuran-5-ylmethylisocyanide.

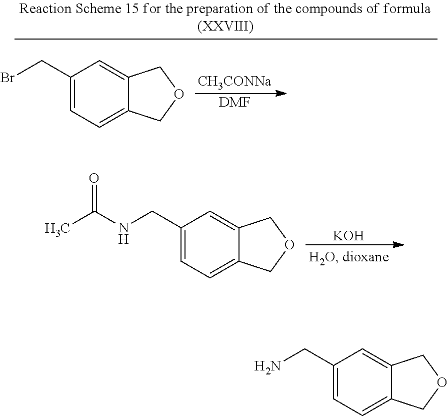

(wherein DMF represents N,N-dimethylformamide and dioxane represents 1,4-dioxane).

Compounds of formula (XXIX) include for example N-{[1-oxo-2-(pyridin-2-ylmethyl)-2,3-dihydro-1H-isoindol-5-yl]methyl}formamide, N-[5-[(formylamino)methyl]-2-(pyridin-2-ylmethyl)-2,3-dihydro-1H-isoindol-1-yliden]acetamide.

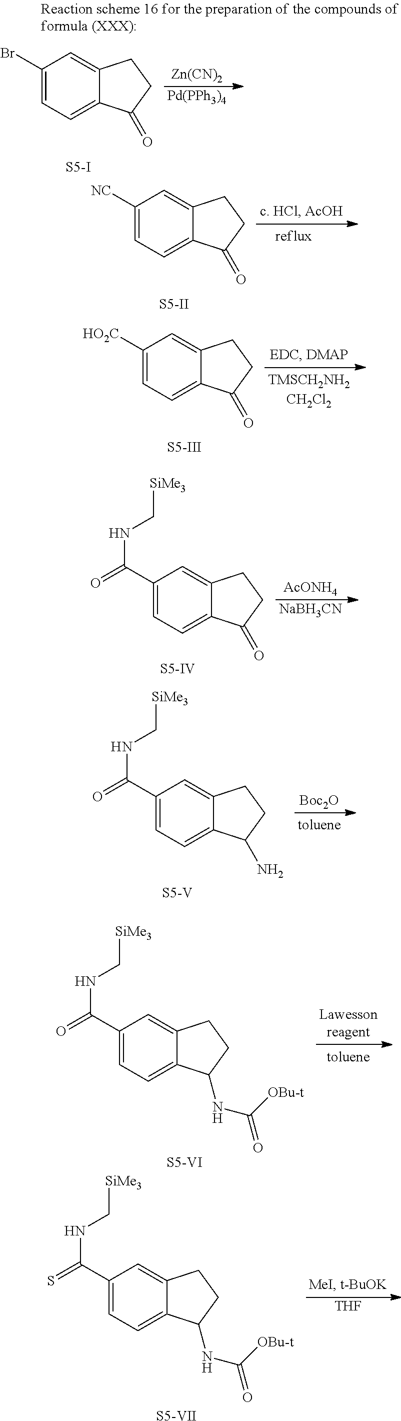

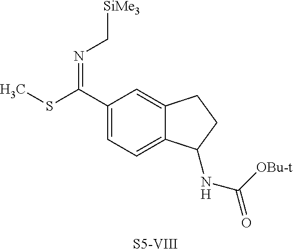

(wherein, Me stands for methyl, (Ph₃P)₄Pd stands for tetrakistriphenylphosphine palladium, c.HCl stands for concentrated hydrochloric acid, Ac stands for an acetyl group, reflux stands for a heating treatment, EDC stands for 1-ethyl-3-(3-dimethylaminopropyl)-carbodiimide, DMAP stands for dimethylaminopyridine, TMS stands for a trimethylsilyl group, Boc rep stands for a t-butoxycarbonyl group, toluene stands for toluene, Lawesson reagent stands for 2,4-bis(4-methoxyphenyl)-1,3-dithia-2,4-diphosphetane-2,4-disulfide, BuO-t represents a t-butoxy group, THF stands for tetrahydrofuran, and t-BuOK stands for t-butoxy potassium).

Compounds of formula (XXX) include for example methyl 1-[(t-butoxycarbonyl)amino-]-N-[(trimethylsilyl)methyl]-2,3-dihydro-1H-inden-5-carboimide thioate, methyl 5-[(t-butoxycarbonyl)amino]-N-[(trimethylsilyl)methyl]-5,6,7,8-tetrahydro-naphthalen-2-carboimide thioate, methyl 5-[(t-butoxycarbonyl)amino]-N-[(trimethylsilyl)-methyl]naphthalen-2-carboimide thioate, methyl 6-[(t-butoxycarbonypamino]-N-[(trimethylsilyl)-methyl]naphthalen-2-carboimide thioate.

Methyl 5-[(t-butoxycarbonyl)amino]-N-[(trimethylsilyl)methyl]-5,6,7,8-tetrahydronaphthalen-2-carboimide thioate can be synthesized according to the method shown in reaction scheme 16, by using 6-bromo-3,4-dihydronaphthalen-1(2H)-one as a starting material.

The compounds of formula (XXX) are intermediates for the preparation of compounds according to the invention. Namely, as compounds according to the invention can be prepared by reacting a compound of formula (XII) with compounds of formula (XXX) in the presence of a fluorine reagent if R₄′ does not stands for H. In case that R₄′ stands for H, the reaction can be carried out in a one pot reaction by firstly adding an alkylating reagent, such as methyl iodide and then adding an appropriate fluorine reagent.

With respect to preparation method (j), once a pyrroline ring part is constructed, substitution with various substituents can be made.

### Reaction Scheme 17 Shows a Synthetic Method Example for Introducing Various Substituents on Condensed-Ring Moiety.

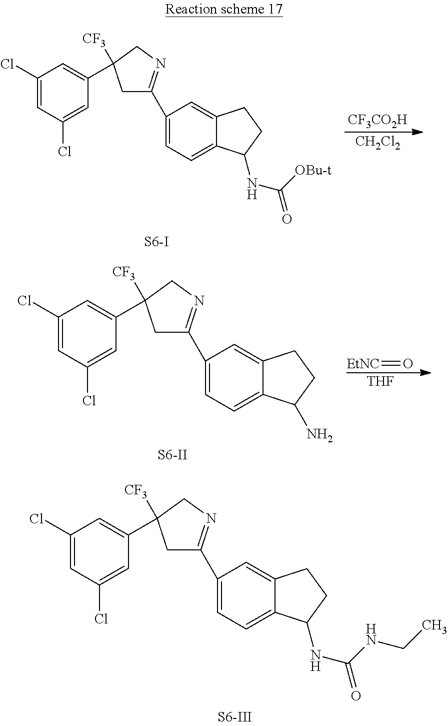

(wherein, Bu-t represents a t-butyl group, Et represents an ethyl group and THF represents tetrahydrofuran).

Appropriate diluents for the preparation methods disclosed herein, particularly for preparation methods (a-1), (a-2), (b), (c), (d), (e), (f), (g), (i) include aliphatic, alicyclic, and aromatic hydrocarbons (which may be chlorinated in some cases), for example, pentane, hexane, cyclo-hexane, petroleum ether, ligroin, benzene, toluene, xylene, chlorobenzene, dichlorobenzene and the like; ethers, for example, ethyl ether, methyl ethyl ether, isopropyl ether, butyl ether, dioxane, dimethoxyethane (DME), tetrahydrofuran (THF), diethylene glycol dimethyl ether (DGM) and the like; ketones, for example, acetone, methyl ethyl ketone (MEK), methyl isopropyl ketone, methyl isobutyl ketone (MIBK) and the like; nitriles, for example, acetonitrile, propionitrile and the like; esters, for example, ethyl acetate, amyl acetate and the like; acid amides, for example, dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethylsulfoxide (DMSO), sulforane and the like; and bases, for example, pyridine and the like.

Appropriate diluents for the preparation method (j) include for example aliphatic, alicyclic, and aromatic hydrocarbons (which may be chlorinated in some cases), for example, pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers, for example, ethyl ether, methyl ethyl ether, isopropyl ether, butyl ether, dioxane, dimethoxyethane (DME), tetrahydrofuran (THF), diethylene glycol dimethyl ether (DGM) and the like; ketones, for example, acetone, methyl ethyl ketone (MEK), methyl isopropyl ketone, methyl isobutyl ketone (MIBK) and the like; nitriles, for example, acetonitrile, propionitrile, acrylonitrile and the like; esters, for example, ethyl acetate, amyl acetate and the like.

Appropriate bases for the preparation methods disclosed herein, particularly for preparation methods (a-1), (a-2), (b), (c), (d), (e), (f), (g) include inorganic bases such as hydrides, hydroxides, carbonates and bicarbonates of alkaline metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide and the like; and organic bases such as alcoholate, tertiary amines, dialkyl-aminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), and 1,8-diazabicyclo[5,4,0]undec-7-ene (DBU) and the like.

Further appropriate bases, particularly for the preparation methods (i) and (h) are alkaline metal bases, such as for example, sodium carbonate, potassium carbonate, sodium hydrogen carbonate, potassium hydrogen carbonate, sodium acetate, potassium acetate, sodium methoxide, sodium ethoxide, potassium-tert-butoxide and the like; and an organic base such as triethylamine, diisopropylethylamine, tributylamine, N-methylmorpholine, N,N-dimethylaniline, N,N-diethylaniline, 4-tert-butyl-N,N-dimethylaniline, pyridine, picoline, lutidine, diazabicyclo-undecene, diazabicyclooctane, imidazole and the like.

The preparation methods disclosed herein, particularly the preparation methods (a-1), (a-2), (b), (c), (d), (e), (f), (g), (i) can be carried out within a substantially broad range of temperatures, i.e. in range of −78° C. to about 200° C. Generally, it can be carried out at a temperature in the range of about 10 to about 150° C., preferably in the range of about 30 to about 120° C. They can be carried out at any pressure, i.e. at a pressure of about 1013 mbar, at a pressure lower than 1013 mbar and a pressure higher than 1013 mbar. Reaction time may vary from about 0.1 hours to about 72 hours, preferably from 1 to 24 hours.

Preparation method (j) can be carried out within a substantially broad range of temperatures. generally, it can be carried out at a temperature in a range of about −78 and about 100° C., preferably in a range of about −10 and about 50° C. In addition, although the above-described reaction is preferably carried out at normal pressure, it can also be carried out under increased or reduced pressure. Reaction time is from 0.1 to 10 hours, and preferably from 1 to 5 hours.

The compounds according to the present invention show a potent insecticidal action and can therefore be used as an insecticide. Furthermore, the compounds according to the present invention exhibit a strong control effect against harmful insects, without imposing any harmful side effects of drug to cultivated plants.

The compounds of the present invention can thus be used for the control of a wide range of pest species, for example, harmful sucking insects, chewing insects, as well as other plant parasitic pests, storage insects, hygiene pests and the like, and can be applied for the purpose of disinfestations and extermination thereof. Harmful animal pest are for example:

As for insects, coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, galleria mellonella, Plutella maculipennis, Heliothis virescens, Phyllocnistis citrella; hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.; thysanopterans, for example, Thrips palmi, Frankinella occidental; orthopterans, for example, Blatella germanica, Periplaneta americana, gryllotalpa Africana, Locusta migratoria migratoriodes; isopterans, for example, Reticulitermes speratus, Coptotermes formosanus; dipterans, for example, Musca domestica, Aedes aegypti, Hylemia platura, Culex pipiens, Anopheles sinensis, Culex tritaeniorhynchus, Liriomyza trifolii.

As for acari, for example, Tetranychus cinnabarinus, Tetranychus urticae, Panonychus citri, Aculops pelekassi, Tarsonemus spp.

As for nematodes, for example, Meloidogyne incognita, Bursaphelenchus lignicolus Mamiya et Kiyohara, Aphelenchoides besseyi, Heterodera glycines, Pratylenchus spp.

Additionally, the compounds according to the present invention show a good plant tolerance and favourable toxicity to warm-blooded animals and being tolerated well by the environment, and thus are suitable for protecting plants and plant parts.

Application of the compounds of the invention may result in increasing the harvest yields, improving the quality of the harvested material. Additionally, the compounds can be used for controlling animal pests, in particular insects, arachnids, helminths, nematodes and molluscs, which are encountered in agriculture, in horticulture, the field of veterinary medicine, in forests, in gardens and leisure facilities, in the protection of stored products and of materials, and in the hygiene sector. They may be preferably employed as plant protection agents. They are active against normally sensitive and resistant species and against all or some stages of development.

These pests include inter alia:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogo-derma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the gastropoda, for example, Arion spp., Biomphalaria spp., Bulinus spp., Deroceras spp., galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Franldiniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof, are treated. The terms “parts”, “parts of plants” and “plant parts” have been explained above.

Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive “synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are the increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD gARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds according to the invention at a suitable concentration.

Furthermore, in the field of veterinary medicine, the novel compounds of the present invention can be effectively used against various harmful animal parasitic pests (endoparasites and ectoparasites), for example, insects and helminthes. Examples of such animal parasitic pests include the pests as described below. Examples of the insects include gasterophilus spp., Stomoxys spp., Trichodectes spp., Rhodnius spp., Ctenocephalides canis, Cimx lecturius, Ctenocephalides felis, Lucilia cuprina, and the like. Examples of acari include Ornithodoros spp., Ixodes spp., Boophilus spp., and the like.

In the veterinary fields, i.e. in the field of veterinary medicine, the active compounds according to the present invention are active against animal parasites, in particular ectoparasites or endoparasites.

The term endoparasites includes in particular helminths, such as cestodes, nematodes or trematodes, and protozoae, such as coccidia. Ectoparasites are typically and preferably arthropods, in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like.

These parasites include:

From the order of the Anoplurida, for example Haematopinus spp., Linognathus spp., Pediculus spp., Phtirus spp., Solenopotes spp.; particular examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;

from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; particular examples are: Bovicola bovis, Bovicola ovis, Bovicola limbata, Damalina bovis, Trichodectes canis, Felicola subrostratus, Bovicola caprae, Lepikentron ovis, Werneckiella equi;

from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., glossina spp., Calliphora spp., Lucilia spp., Chrysomyia spp., Wohlfahrtia spp., Sarcophaga spp., Oestrus spp., Hypoderma spp., gasterophilus spp., Hippobosca spp., Lipoptena spp., Melophagus spp., Rhinoestrus spp., Tipula spp.; particular examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, gasterophilus intestinalis, gasterophilus haemorroidalis, gasterophilus inermis, gasterophilus nasalis, gasterophilus nigricornis, gasterophilus pecorum, Braula coeca;

from the order of the Siphonapterida, for example Pulex spp., Ctenocephalides spp., Tunga spp., Xenopsylla spp., Ceratophyllus spp.; particular examples are: Ctenocephalides canis, Ctenocephalides felis, Pulex irritans, Tunga penetrans, Xenopsylla cheopis;

from the order of the Heteropterida, for example Cimex spp., Triatoma spp., Rhodnius spp., Panstrongylus spp.

From the order of the Blattarida, for example Blatta orientalis, Periplaneta americana, Blattela germanica, Supella spp. (e.g. Suppella longipalpa);

From the subclass of the Acari (Acarina) and the orders of the Meta- and Mesostigmata, for example Argas spp., Ornithodorus spp., Otobius spp., Ixodes spp., Amblyomma spp., Rhipicephalus (Boophilus) spp Dermacentor spp., Haemophysalis spp., Hyalomma spp., Dermanyssus spp., Rhipicephalus spp. (the original genus of multi host ticks) Ornithonyssus spp., Pneumonyssus spp., Raillietia spp., Pneumonyssus spp., Sternostoma spp., Varroa spp., Acarapis spp.; particular examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni;

from the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particular examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neoschongastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds according to the invention are also suitable for controlling arthropods, helminths and protozoae, which attack animals. Animals include agricultural livestock such as, for example, cattle, sheep, goats, horses, pigs, donkeys, camels, buffaloes, rabbits, chickens, turkeys, ducks, geese, cultured fish, honeybees. Moreover, animals include domestic animals—also referred to as companion animals—such as, for example, dogs, cats, cage birds, aquarium fish and what are known as experimental animals such as, for example, hamsters, guinea pigs, rats and mice.

By controlling these arthropods, helminths and/or protozoae, it is intended to reduce deaths and improve performance (in the case of meat, milk, wool, hides, eggs, honey and the like) and health of the host animal, so that more economical and simpler animal keeping is made possible by the use of the active compounds according to the invention.

For example, it is desirable to prevent or interrupt the uptake of blood by the parasites from the hosts (when applicable). Also, controlling the parasites may help to prevent the transmittance of infectious agents.

The term “controlling” as used herein with regard to the veterinary field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

According to a preferred embodiment the compounds of the present invention are used in the control of ectoparasitic arthropods (as mentioned above) on animals, namely on agricultural livestock or on domestic animals.

Generally, when used for the treatment of animals the active compounds according to the invention can be applied directly. Preferably they are applied as pharmaceutical compositions which may contain pharmaceutically acceptable excipients and/or auxiliaries which are known in the art.

In the veterinary field and in animal keeping, the active compounds are applied (=administered) in the known manner by enteral administration in the form of, for example, tablets, capsules, drinks, drenches, granules, pastes, boluses, the feed-through method, suppositories; by parenteral administration, such as, for example, by injections (intramuscular, subcutaneous, intravenous, intraperitoneal and the like), implants, by nasal application, by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like. The active compounds may be formulated as shampoo or as suitable formulations usable in aerosols, unpressurized sprays, for example pump sprays and atomizer sprays.

When used for livestock, poultry, domestic animals and the like, the active compounds according to the invention can be applied as formulations (for example powders, wettable powders [“WP”], emulsions, emulsifiable concentrates [“EC”], flowables, homogeneous solutions, and suspension concentrates [“SC”]) which comprise the active compounds in an amount of from 1 to 80% by weight, either directly or after dilution (e.g. 100- to 10 000-fold dilution), or else as a chemical bath.

When used in the veterinary field the active compounds according to the invention may be used in combination with suitable synergists or other active compounds, such as for example, acaricides, insecticides, anthelmintics, anti-protozoal drugs.

In the present invention, a substance having an insecticidal action against pests including all of these is referred to as an insecticide.

An active compound of the present invention can be prepared in conventional formulation forms, when used as an insecticide. Examples of the formulation forms include solutions, emulsions, wettable powders, water dispersible granules, suspensions, powders, foams, pastes, tablets, granules, aerosols, active compound-infiltrated natural and synthetic materials, microcapsules, seed coating agents, formulations used with a combustion apparatus (for example, fumigation and smoking cartridges, cans, coils or the like as the combustion apparatus), ULV (cold mist, warm mist), and the like.

These formulations can be produced by methods that are known per se. For example, a formulation can be produced by mixing the active compound with a developer, that is, a liquid diluent or carrier; a liquefied gas diluent or carrier; a solid diluent or carrier, and optionally with a surfactant, that is, an emulsifier and/or dispersant and/or foaming agent.

In the case where water is used as the developer, for example, an organic solvent can also be used as an auxiliary solvent.

Examples of the liquid diluent or carrier include aromatic hydrocarbons (for example, xylene, toluene, alkylnaphthalene and the like), chlorinated aromatic or chlorinated aliphatic hydrocarbons (for example, chlorobenzenes, ethylene chlorides, methylene chlorides), aliphatic hydrocarbons (for example, cyclohexanes), paraffins (for example, mineral oil fractions), alcohols (for example, butanol, glycols and their ethers, esters and the like), ketones (for example, acetone, methyl ethyl ketone, methyl isobutyl ketone, cyclohexanone and the like), strongly polar solvents (for example, dimethylformamide, dimethylsulfoxide and the like), water and the like.

The liquefied gas diluent or carrier may be those which are gaseous at normal temperature and normal pressure, for example, aerosol propellants such as butane, propane, nitrogen gas, carbon dioxide and halogenated hydrocarbons.

Examples of the solid diluent include pulverized natural minerals (for example, kaolin, clay, talc, chalk, quartz, attapulgite, montmorillonite, diatomaceous earth, and the like), pulverized synthetic minerals (for example, highly dispersed silicic acid, alumina, silicates and the like), and the like.

Examples of the solid carrier for granules include pulverized and screened rocks (for example, calcite, marble, pumice, sepiolite, dolomite and the like), synthetic granules of inorganic and organic powder, fine particles of organic materials (for example, sawdust, coconut shells, maize cobs, tobacco stalk and the like), and the like.

Examples of the emulsifier and/or foaming agent include nonionic and anionic emulsifiers [for example, polyoxyethylene fatty acid esters, polyoxyethylene fatty acid alcohol ethers (for example, alkylaryl polyglycol ether), alkylsulfonates, alkylsulfates, arylsulfonates and the like], albumin hydrolyzate, and the like.

Examples of the dispersant include lignin sulfite waste liquor and methylcellulose.

Fixing agents can also be used in the formulations (powders, granules, emulsions), and examples of the fixing agent include carboxymethylcellulose, natural and synthetic polymers (for example, gum arabic, polyvinyl alcohol, polyvinyl acetate, and the like) and the like.

Colorants can also be used, and examples of the colorants include inorganic pigments (for example, iron oxide, titanium oxide, Prussian Blue and the like), organic dyes such as alizarin dyes, azo dyes or metal phthalocyanine dyes, and in addition, trace elements such as the salts of iron, manganese, boron, copper, cobalt, molybdenum and zinc.

The formulations in general can contain the active ingredient in an amount ranging from 0.1 to 95% by weight, and preferably 0.5 to 90% by weight.

The compound according to the present invention can also exist as an admixture with other active compounds, for example, insecticides, poisonous baits, bactericides, miticides, nematicides, fungicides, growth regulators, herbicides and the like, in the form of their commercially useful formulation forms and in the application forms prepared from those formulations.

The content of the compound according to the present invention in a commercially useful application form can be varied within a wide range.

The concentration of the active compound according to the present invention in actual usage can be, for example, in the range of 0.0000001 to 100% by weight, and preferably 0.00001 to 1% by weight.

The compounds according to the present invention can be used through conventional methods that are appropriate for the usage form.

The active compound of the present invention have, when used against hygiene pests and pests associated with stored products, stability effective against alkali on lime materials, and also shows excellent residual effectiveness on wood and soil.

Next, the present invention is exemplified by way of the following examples, but the invention is not intended to be limited thereto. References to room temperature means temperatures of about 18 to about 30° C.

### A: Synthesis of 2-acetyl-5-[5-(3,5-dichlorophenyl)-5-(trifluoromethyl)-4,5-dihydroisoxazol-3-yl]-2,3-dihydro-1H-isoindol-1-one (Compound No. 1-221)

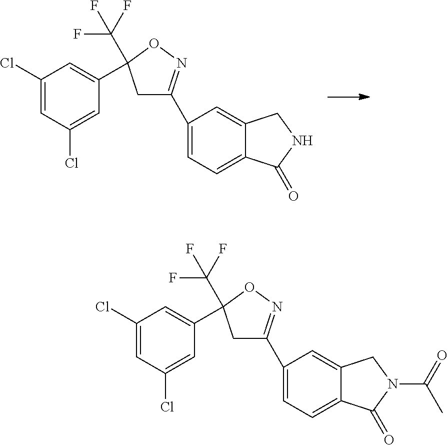

5-[5-(3,5-dichlorophenyl)-5-(trifluoromethyl)-4,5-dihydroisoxazol-3-yl]-2,3-dihydro-1H-isoindol-1-one (100 mg) was heated and refluxed in anhydrous acetic acid (246 mg) for 2 hours. After removing anhydrous acetic acid by distillation under a reduced pressure, the mixture was diluted with an appropriate amount of t-butyl methyl ether, washed with water and a saturated saline solution, and dried over magnesium sulfate. After separation by filtration, the solvent was distilled off under a reduced pressure and the resulting residue was purified with silica gel chromatography to obtain 2-acetyl-5-[5-(3,5-dichlorophenyl)-5-(trifluoromethyl)-4,5-dihydroisoxazol-3-yl]-2,3-dihydro-1H-isoindol-1-one (70 mg) in 63% yield.

¹H-NMR (CDCl₃) δ: 2.70 (3H, s), 3.76 (1H, d, J=17.0 Hz), 4.14 (1H, d, J=17.4 Hz), 4.85 (2H, s), 7.44-7.45 (1H, m), 7.51-7.52 (2H, m), 7.81-7.83 (2H, m), 7.96-7.99 (1H, m).

### B: Synthesis of 5-[5-(3,5-dichlorophenyl)-5-(trifluoromethyl)-4,5-dihydroisoxazol-3-yl]-2-benzofuran-1(3H)-one (Compound No. 1-2)

### Step 1. Synthesis of methyl 2-[(acetyloxy)methyl]-4-[5-(3,5-dichlorophenyl)-5-(trifluoromethyl)-4,5-dihydroisoxazol-3-yl]benzoate

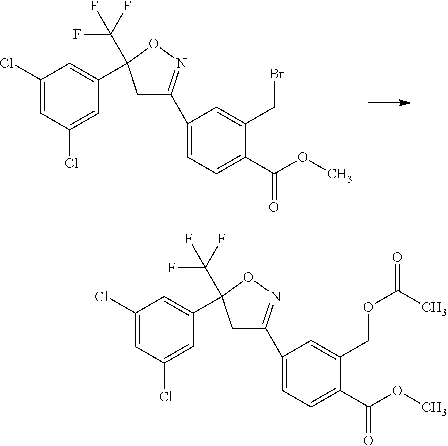

Methyl 2-(bromomethyl)-4-[5-(3,5-dichlorophenyl)-5-(trifluoromethyl)-4,5-dihydro-oxazol-3-yl]benzoate (887 mg) and sodium acetate (285 mg) were dissolved in DMF (10 ml) and then stirred at 70° C. for 5 hours. After cooled to room temperature, the mixture was diluted with an appropriate amount of t-butyla methyl ether, washed with water three times and with a saturated saline solution, and dried over magnesium sulfate. After separation by filtration, the solvent was distilled off under a reduced pressure to obtain methyl 2-[(acetyloxy)methyl]-4-[5-(3,5-dichlorophenyl)-5-(trifluoromethyl)-4,5-dihydroisoxazol-3-yl]-benzoate (850 mg) in 99% crude yield.

¹H-NMR (CDCl₃) δ: 2.17 (3H, s), 3.73 (1H, d), 3.93 (3H, s), 4.12 (1H, d), 5.52 (2H, s), 7.43-7.44 (1H, m), 7.52-7.52 (2H, m), 7.63-7.65 (1H, m), 7.83-7.83 (1H, m), 8.02-8.05 (1H, m).

### Step 2. Synthesis of 5-[5-(3,5-dichlorophenyl)-5-(trifluoromethyl)-4,5-dihydroisoxazol-3-yl]-2-benzofuran-1(3H)-one (Compound No. 1-2)

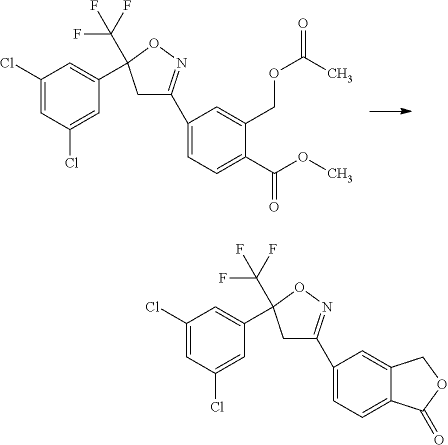

Methyl 2-[(acetyloxy)methyl]-4-[5-(3,5-dichlorophenyl)-5-(trifluoromethyl)-4,5-dihydro-isoxazol-3-yl]benzoate (851 mg) and sodium methoxide (9 mg) were stirred in methanol (10 ml) at room temperature for 30 min. Under a reduced pressure, the solvent was removed by distillation. Then, the residue was diluted with an appropriate amount of t-butyl methyl ether, washed with water and a saturated saline solution, and dried over magnesium sulfate. After separation by filtration, the solvent was distilled off under a reduced pressure and the resulting residue was purified with silica gel chromatography to obtain 5-[5-(3,5-dichlorophenyl)-5-(trifluoromethyl)-4,5-dihydroisoxazol-3-yl]-2-benzofuran-1(3H)-one (140 mg) in 19% yield.

¹H-NMR (CDCl₃) δ: 3.94 (2H, m), 5.36 (2H, s), 7.44-7.51 (3H, m), 7.78-8.04 (3H, m).

### C: Synthesis of N-[(1E)-5-[5-(3,5-dichlorophenyl)-5-(trifluoromethyl)-4,5-dihydroisoxazol-3-yl]-2-(pyridin-2-ylmethyl)-2,3-dihydro-1H-isoindol-1-ylinden]acetamide (Compound No. 1-235)

### Step 1. Synthesis of 4-[5-(3,5-dichlorophenyl)-5-(trifluoromethyl)-4,5-dihydroisoxazol-3-yl]-2-methylbenzonitrile

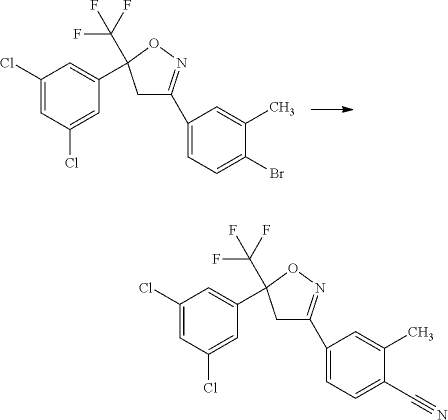

3-(4-bromo-3-methylphenyl)-5-(3,5-dichlorophenyl)-5-(trifluoromethyl)-4,5-dihydro-isoxazole (5.10 g) was dissolved in DMF (10 ml), and under an argon atmosphere zinc cyanide (0.93 g) and tetrakis(triphenylphosphine)palladium (1.30 g) were added thereto. The mixture was then stirred at 80° C. for 4 hours. After cooled to room temperature, the mixture was diluted with an appropriate amount of t-butyl methyl ether, washed with water and dried over magnesium sulfate. After separation by filtration, the solvent was distilled off under a reduced pressure and the resulting crystal was washed with hexane to obtain 4-[5-(3,5-dichlorophenyl)-5-(trifluoromethyl)-4,5-dihydroisoxazol-3-yl]-2-methylbenzonitrile (3.0 g) in 66% yield.

¹H-NMR (CDCl₃) δ: 2.59 (3H, s), 3.69 (1H, d), 4.07 (1H, d), 7.43-7.44 (1H, m), 7.50-7.50 (2H, m), 7.56-7.58 (1H, m), 7.63-7.67 (2H, m).

### Step 2. Synthesis of 2-(bromomethyl)-4-[5-(3,5-dichlorophenyl)-5-(trifluoromethyl)-4,5-dihydro-isoxazol-3-yl]benzonitrile

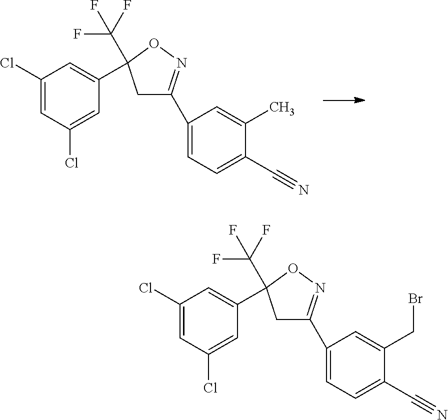

A dichloroethane solution (38 ml) of 4-[5-(3,5-dichlorophenyl)-5-(trifluoromethyl)-4,5-dihydroisoxazol-3-yl]-2-methylbenzonitrile (1.0 g), N-bromosuccinimide (0.62 g) and a catalytic amount of 2,2′-azobisisobutyronitrile (MEIN) was heated and refluxed for 3 hours. After the solution was cooled to room temperature, insoluble matters were filtered out and the solvent was distilled off under a reduced pressure. The residue was dissolved in t-butyl methyl ether, then washed with water, and dried over magnesium sulfate. After separation by filtration again, the solvent was distilled off under a reduced pressure to obtain 2-(bromomethyl)-4-[5-(3,5-dichlorophenyl)-5-(trifluoromethyl)-4,5-dihydroisoxazol-3-yl]benzonitrile as a crude product. The crude product thus obtained was used for a further reaction without any purification.

¹H-NMR (CDCl₃) δ: 3.71 (1H, d), 4.09 (1H, d), 4.64 (2H, s), 7.45-7.48 (3H, m), 7.72-7.73 (2H, m), 7.83-7.86 (1H, m).

### Step 3. Synthesis of 5-[5-(3,5-dichlorophenyl)-5-(trifluoromethyl)-4,5-dihydroisoxazol-3-yl]-2-(pyridin-2-ylmethyl)-2,3-dihydro-1H-isoindol-1-imine

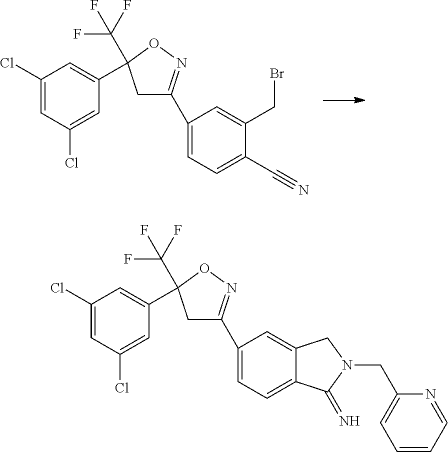

2-(bromomethyl)-4-[5-(3,5-dichlorophenyl)-5-(trifluoromethyl)-4,5-dihydro-isoxazol-3-yl]benzonitrile (500 mg), 2-pyridylmethylamine (113 mg) and potassium carbonate (289 mg) in acetonitrile (10 ml) were heated and refluxed for 3 hours. After cooled to room temperature, the mixture was diluted with an appropriate amount of t-butyl methyl ether, washed with water and a saturated saline solution, and dried over magnesium sulfate. After separation by filtration, the solvent was distilled off under a reduced pressure and the resulting residue was purified with silica gel chromatography to obtain 5-[5-(3,5-dichlorophenyl)-5-(trifluoromethyl)-4,5-dihydro-isoxazol-3-yl]-2-(pyridin-2-ylmethyl)-2,3-dihydro-1H-isoindol-1-imine (400 mg) in 75% yield.

### Step 4. Synthesis of N-[(1E)-5-[5-(3,5-dichlorophenyl)-5-(trifluoromethyl)-4,5-dihydro-isoxazol-3-yl]-2-(pyridin-2-ylmethyl)-2,3-dihydro-1H-isoindol-1-ylinden]acetamide (Compound No. 1-235)

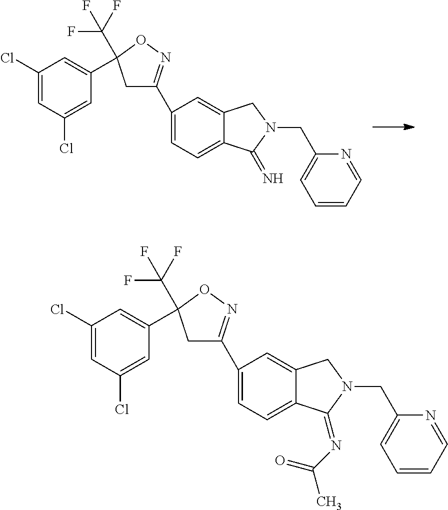

Acetyl chloride (94 mg) and pyridine (119 mg) were added to 5-[5-(3,5-dichlorophenyl)-5-(trifluoromethyl)-4,5-dihydroisoxazol-3-yl]-2-(pyridin-2-ylmethyl)-2,3-dihydro-1H-isoindol-1-imine (505 mg) in tetrahydrofuran (5 ml) and the mixture was stirred at room temperature for 1 hour. Then, the resulting mixture was diluted with an appropriate amount of t-butyl methyl ether, washed with water and a saturated saline solution, and dried over magnesium sulfate. After separation by filtration, the solvent was distilled off under a reduced pressure and the resulting residue was purified with silica gel chromatography to obtain N-[(1E)—5-[5-(3,5-dichlorophenyl)-5-(trifluoromethyl)-4,5-dihydroisoxazol-3-yl]-2-(pyridin-2-ylmethyl)-2,3-dihydro-1H-isoindol-1-ylinden]acetamide (80 mg) in 15% yield.

¹H-NMR (CDCl₃) δ: 2.12-2.24 (3H, m), 3.60-3.68 (1H, m), 4.02-4.08 (1H, m), 4.73-4.82 (4H, m), 7.13-7.73 (9H, m), 8.44-8.61 (1H, m).

### D: Synthesis of 1-{5-[5-(3,5-dichlorophenyl)-5-(trifluoromethyl)-4,5-dihydroisoxazol-3-yl]-1,3-dihydro-2H-isoindol-2-yl}ethanone (Compound No. 1-151)

### Step 1. Synthesis of 3-[3,4-bis(bromomethyl)phenyl]-5-(3,5-dichlorophenyl)-5-(trifluoromethyl)-4,5-dihydroisoxazole

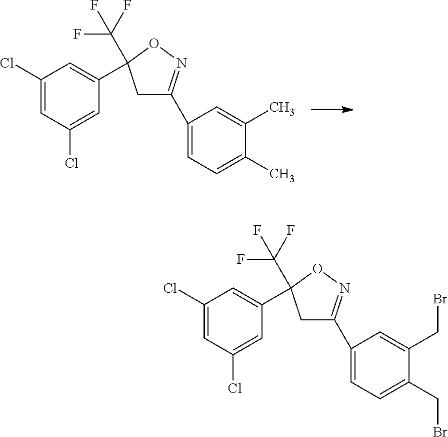

A dichloroethane solution (38 ml) of 5-(3,5-dichlorophenyl)-3-(3,4-dimethylphenyl)-5-(trifluoromethyl)-4,5-dihydroisoxazole (1.0 g), N-bromosuccinimide (1.1 g) and a catalytic amount of 2,2′-azobisisobutyronitrile (AIBN) was heated and refluxed for 3 hours. After the solution was cooled to room temperature, insoluble matters were filtered out and the solvent was distilled off under a reduced pressure. The residue was dissolved in t-butyl methyl ether, then washed with water, and dried over magnesium sulfate. After separation by filtration again, the solvent was distilled off under a reduced pressure to obtain 3-[3,4-bis(bromomethyl)phenyl]-5-(3,5-dichlorophenyl)-5-(trifluoromethyl)-4,5-dihydroisoxazole as a crude product. The crude product thus obtained was used for a further reaction without any purification.

¹H-NMR (CDCl₃) δ: 3.66-3.71 (1H, m), 4.04-4.11 (1H, m), 4.64 (2H, s), 7.41-7.67 (6H, m).

### Step 2. Synthesis of 1-{5-[5-(3,5-dichlorophenyl)-5-(trifluoromethyl)-4,5-dihydroisoxazol-3-yl]-1,3-dihydro-2H-isoindol-2-yl}ethanone (Compound No. 1-151).

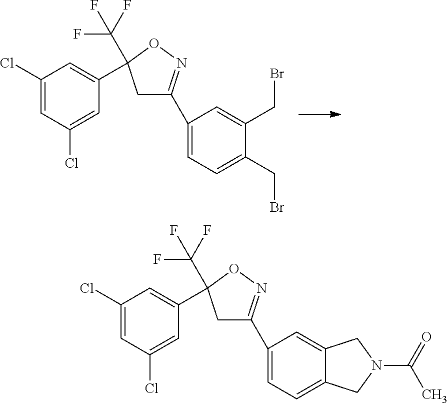

3-[3,4-bis(bromomethyl)phenyl]-5-(3,5-dichlorophenyl)-5-(trifluoromethyl)-4,5-dihydro-isoxazole (1.41 g), acetamide (0.15 g) and sodium hydride (0.10 g) were heated and refluxed in tetrahydrofuran solution (30 ml) for 3 hours. After cooled to room temperature, the mixture was diluted with an appropriate amount of t-butyl methyl ether, washed with water and a saturated saline solution, and dried over magnesium sulfate. After separation by filtration, the solvent was distilled off under a reduced pressure and the resulting residue was purified with silica gel chromatography to obtain 1-{5-[5-(3,5-dichlorophenyl)-5-(trifluoromethyl)-4,5-dihydro-isoxazol-3-yl]-1,3-dihydro-2H-isoindol-2-yl}ethanone (100 mg) in 8.8% yield.

¹H-NMR (CDCl₃) δ: 2.18 (3H, s), 3.71 (1H, d), 4.10 (1H, d), 4.82-4.85 (4H, m), 7.32-7.39 (1H, m), 7.43-7.43 (1H, m), 7.52-7.65 (4H, m).

### E: 5-(3,5-dichlorophenyl)-3-(1,3-dihydro-2-benzofuran-5-yl)-5-(trifluoromethyl)-4,5-dihydroisoxazole (Compound No. 1-131)

### Step 1. Synthesis of 2,5-dimethylbenzyl acetate

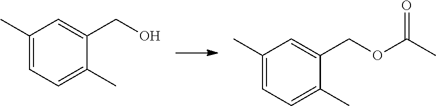

Acetyl chloride (1.5 g) was added to a tetrahydrofuran solution (30 ml) including 2,5-dimethylbenzyl alcohol and triethylamine (2.4 g) under ice cooling. After stirring at room temperature for 1 hour, the reaction solution was diluted with t-butyl methyl ether. The solution was then washed with water and a saturated saline solution, and the organic layer was dried over anhydrous magnesium sulfate. The solvent was distilled off under a reduced pressure to obtain 2,5-dimethylbenzyl acetate (2.8 g).

¹H-NMR (CDCl₃) δ: 2.09 (3H, s), 2.31 (6H, d), 5.09 (2H, s), 7.08-7.12 (3H, m).

### Step 2. Synthesis of 5-(bromomethyl)-1,3-dihydro-2-benzofuran

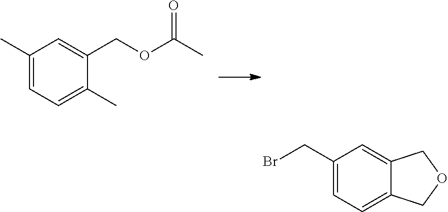

A dichloroethane solution (30 ml) of 2,5-dimethylbenzyl acetate (3 g), 2,2′-azobisisobutyronitrile (AIBN)(0.2 g) and N-bromosuccinimide (6.8 g) was stirred at 90° C. for 3 hours. Under a reduced pressure, the reaction solution was concentrated, t-butyl methyl ether was added to the residue, and separation was carried out by filtration. The filtered solution was washed with water and a saturated saline solution, and the organic layer was dried over magnesium sulfate. The solvent was distilled off under a reduced pressure and the resulting residue was purified with silica gel chromatography to obtain 2,5-bis(bromomethyl)benzyl acetate (2.0 g), to which ethanol (10 ml) and water (5 ml) were added followed by the addition of sodium hydroxide (0.5 g). The mixture was stirred at room temperature for 1 hour. The reaction solution was then diluted with t-butyl methyl ether, washed with water and a saturated saline solution, and then the organic layer was dried over anhydrous magnesium sulfate. The solvent was distilled off under a reduced pressure and the resulting residue was purified with silica gel chromatography to obtain 5-(bromomethyl)-1,3-dihydro-2-benzofuran (0.65 g).

¹H-NMR (CDCl₃) δ: 4.52 (2H, s), 5.10 (4H, s), 7.19-7.31 (3H, m).

### Step 3. Synthesis of 1,3-dihydro-2-benzofuran-5-carbaldehyde

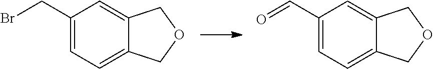

A N,N-dimethylformamide solution (10 ml) of 5-(bromomethyl)-1,3-dihydro-2-benzofuran (0.7 g) and sodium acetate (0.54 g) was stirred at 70° C. for 3 hours. The reaction solution was diluted with t-butyl methyl ether, then washed with water and a saturated saline solution, and the organic layer was dried over anhydrous magnesium sulfate. The solvent was distilled off under a reduced pressure to obtain 1,3-dihydro-2-benzofuran-5-ylmethyl acetate as a crude product. The crude product thus obtained was dissolved in methanol (10 ml), sodium methoxide (0.05 g) was added thereto and the mixture was stirred for 1 hour at room temperature. The reaction solution was diluted with t-butyl methyl ether, then washed with water and a saturated saline solution, and the organic layer was dried over magnesium sulfate. The solvent was distilled off under a reduced pressure to obtain 1,3-dihydro-2-benzofuran-5-ylmethanol as a crude product. The crude product thus obtained was dissolved in methylene chloride (20 ml), activated manganese (IV) oxide (2.3 g) was added thereto and the mixture was heated and refluxed for 5 hours. The reaction solution was filtered using Celite and the filtered solution was concentrated under a reduced pressure. The resulting residue was purified with silica gel chromatography to obtain 1,3-dihydro-2-benzofuran-5-carbaldehyde (0.35 g).

¹H-NMR (CDCl₃) δ: 5.16 (4H, s), 7.39-7.41 (1H, m), 7.78-7.80 (2H, m), 10.02 (1H, s).

### Step 4. Synthesis of 5-(3,5-dichlorophenyl)-3-(1,3-dihydro-2-benzofuran-5-yl)-5-(trifluoromethyl)-4,5-dihydroisoxazole (Compound No. 1-131)

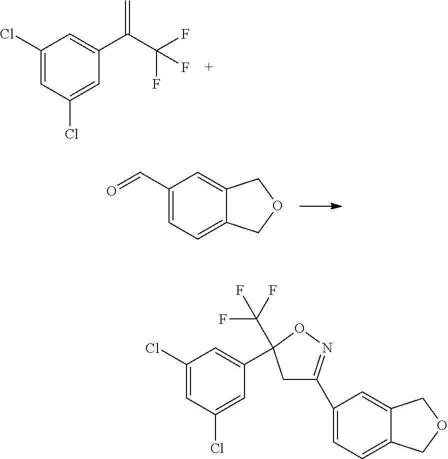

Ethanol (10 ml) and water (6 ml) solution including 1,3-dihydro-2-benzofuran-5-carbaldehyde (0.4 g), hydroxyamine hydrochloride (0.28 g) and sodium acetate (0.45 g) was stirred at room temperature for 1 hour. The reaction solution was diluted with t-butyl methyl ether, then washed with water and a saturated saline solution, and then the organic layer was dried over anhydrous magnesium sulfate. The solvent was distilled off under a reduced pressure to obtain 1-(1,3-dihydro-2-benzofuran-5-yl)-N-hydroxymethanimine (0.2 g) as a crude product. The crude product thus obtained was dissolved in N,N-dimethylformamide (10 ml), N-chlorosuccinimide (0.18 g) was added thereto, and the mixture was stirred at room temperature for 2 hours. To the reaction solution, 1,3-dichloro-5-[1-(trifluoromethyl)vinyl]benzene (0.2 g) was added. After cooled to 0° C., potassium hydrogen carbonate (0.1 g) was added thereto and the mixture was stirred at room temperature for 8 hours. After adding water, extraction was carried out using t-butyl methyl ether. The organic layer was washed with a saturated saline solution, and then dried over magnesium sulfate. The solvent was distilled off under a reduced pressure and the resulting residue was purified with silica gel chromatography to obtain 5-(3,5-dichlorophenyl)-3-(1,3-dihydro-2-benzofuran-5-yl)-5-(trifluoromethyl)-4,5-dihydroisoxazole (0.14 g).

¹H-NMR (CDCl₃) δ: 3.72 (1H, d), 4.11 (1H, d), 5.11 (4H, s), 7.27-7.30 (1H, m), 7.41-7.41 (1H, m), 7.51-7.57 (4H, m).

### F: Synthesis of 5-[5-(3,5-dichlorophenyl)-5-(trifluoromethyl)-4,5-dihydroisoxazol-3-yl]-2-benzofuran-1(3H)-imine (Compound No. 1-232)

### Step 1. Synthesis of 2-cyano-5-[5-(3,5-dichlorophenyl)-5-(trifluoromethyl)-4,5-dihydro-isoxazol-3-yl]benzyl acetate

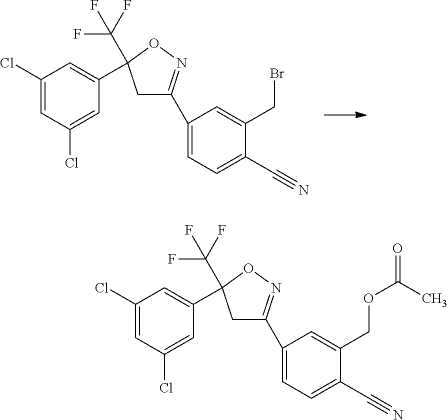

2-(bromomethyl)-4-[5-(3,5-dichlorophenyl)-5-(trifluoromethyl)-4,5-dihydro-isoxazol-3-yl]benzonitrile (900 mg) and sodium acetate (300 mg) were dissolved in N,N-dimethylformamide (10 ml) and the mixture was stirred at 70° C. for 5 hours. After cooled to room temperature, the mixture was diluted with an appropriate amount of t-butyl methyl ether, washed with water three times and with a saturated saline solution, and dried over magnesium sulfate. After separation by filtration, the solvent was distilled off under a reduced pressure and the resulting residue was purified with silica gel chromatography to obtain 2-cyano-5-[5-(3,5-dichlorophenyl)-5-(trifluoromethyl)-4,5-dihydroisoxazol-3-yl]benzyl acetate (250 mg) in 30% yield.

¹H-NMR (CDCl₃) δ: 2.16 (3H, s), 3.72 (1H, d), 4.10 (1H, d), 5.30 (2H, s), 7.44-7.44 (1H, m), 7.48-7.52 (2H, m), 7.72-7.78 (2H, m), 7.81-7.84 (1H, m).

### Step 2. Synthesis of 5-[5-(3,5-dichlorophenyl)-5-(trifluoromethyl)-4,5-dihydroisoxazol-3-yl]-2-benzofuran-1(3H)-imine (Compound No. 1-232)

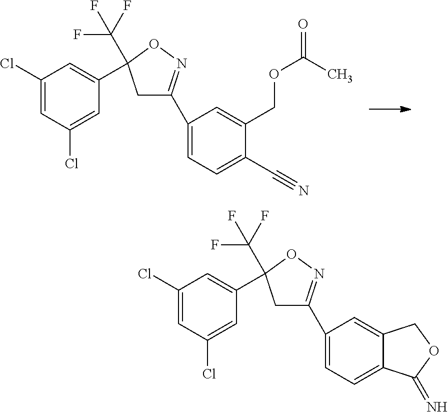

2-cyano-5-[5-(3,5-dichlorophenyl)-5-(trifluoromethyl)-4,5-dihydroisoxazol-3-yl]benzyl acetate (300 mg) and sodium methoxide (10 mg) in methanol (10 ml) was stirred at room temperature for 30 min. After removing the solvent by distillation under a reduced pressure, the mixture was diluted with an appropriate amount of t-butyl methyl ether, washed with water and a saturated saline solution, and dried over magnesium sulfate. After separation by filtration, the solvent was distilled off under a reduced pressure to obtain 5-[5-(3,5-dichlorophenyl)-5-(trifluoromethyl)-4,5-dihydroisoxazol-3-yl]-2-benzofuran-1(3H)-imine (200 mg) in 74% yield.

¹H-NMR (CDCl₃) δ: 3.74 (1H, d), 4.12 (1H, d), 5.34 (2H, s), 7.44-7.44 (1H, m), 7.49-7.52 (2H, m), 7.71-7.73 (1H, m), 7.76-7.79 (1H, m), 7.92-7.95 (1H, m).

### G: Synthesis of N-{5-[5-(3,5-dichlorophenyl)-5-(trifluoromethyl)-4,5-dihydroisoxazol-3-yl]-2,3-dihydro-1H-inden-1-yl}acetamide (No. 3-3)

### Step 1. Synthesis of methyl (2E)-3-(1-oxo-2,3-dihydro-1H-inden-5-yl)prop-2-enoate

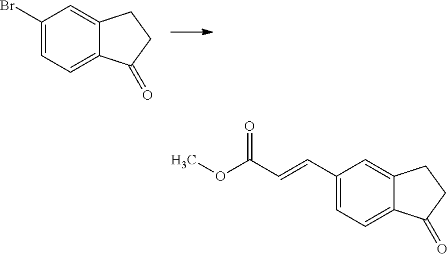

5-bromoindanone (10 g), methyl acrylate (8.56 ml), and 1,3-bis(diphenyl-phosphino)propane (1.17 g) were dissolved in triethylamine (100 ml)-acetonitrile (100 ml), and palladium acetic acid (0.53 g) was added thereto under argon atmosphere. The reaction solution was heated at 80° C. for 8 hours. After the cooling, methyl acrylate (4.28 ml) was further added to the mixture. It was again heated at 80° C. for 8 hours, and the solvent was distilled off under reduced pressure. Dilute hydrochloric acid and methylene chloride were added for the extraction. The organic layer was dried over magnesium sulfate and the solvent was removed by distillation. After the purification with silica gel chromatography, the title compound was obtained (5.36 g, 52%).

¹H-NMR (CDCl₃) δ: 2.73 (t, 2H), 3.17 (t, 2H), 3.83 (s, 3H), 6.55 (d, 1H), 7.50-7.82 (m, 4H).

### Step 2. Synthesis of 1-oxo-2,3-dihydro-1H-inden-5-carbaldehyde

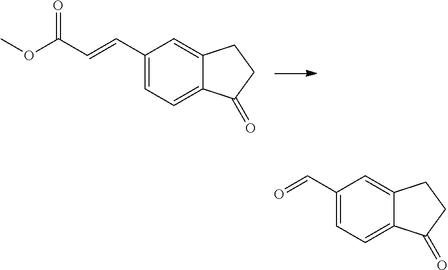

Methyl (2E)-3-(I-oxo-2,3-dihydro-1H-inden-5-yl)prop-2-enoate was dissolved in 1,2-dichloroethane (70 ml)-water (70 ml), and sodium periodate (13.25 g) and ruthenium trichloride hydrates (0.18 g) were added thereto at room temperature. The reaction solution was stirred for 4 hours. Sodium thiosulfate and ethyl acetate were added for the extraction. The organic layer was dried over magnesium sulfate and the solvent was removed by distillation. After the purification with silica gel chromatography, the title compound was obtained (1.92 g, 50%).

¹H-NMR (CDCl₃) δ: 2.78 (t, 2H), 3.25 (t, 2H), 7.90 (s, 2H), 8.00 (s, 1H), 10.14 (s, 1H).

### Step 3. Synthesis of 5[(E)-(hydroxyimino)methyl]-2,3-dihydro-1H-inden-1-one

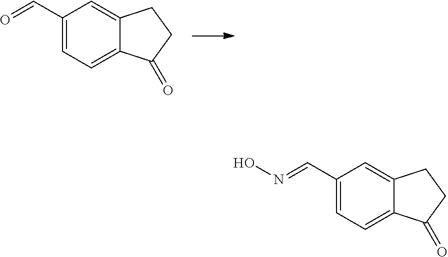

1-oxo-2,3-dihydro-1H-inden-5-carbaldehyde (2.00 g) and sodium hydrocarbonate (1.36 g) were suspended in ethanol, and hydroxyamine hydrochloride (0.87 g) was added thereto at 0° C. After stirring for 1 hour, the solvent was removed by distillation. Water and ethyl acetate were added for the extraction. The organic layer was dried over magnesium sulfate and the solvent was removed by distillation. The title compound was obtained as a crude product (1.92 g).

¹H-NMR (CDCl₃) δ: 2.73 (t, 2H), 3.17 (t, 2H), 7.61 (d, 1H), 7.68 (s, 1H), 7.77 (d, 1H), 8.19 (s, 1H).

### Step 4. Synthesis of 5-[5-(3,5-dichlorophenyl)-5-(trifluoromethyl)-4,5-dihydroisoxazol-3-yl]-2,3-dihydro-1H-inden-1-one

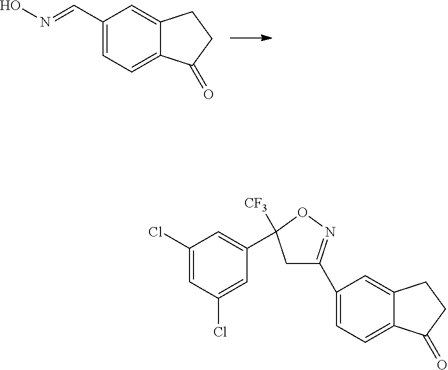

5-[(E)-(hydroxyimino)methyl]-2,3-dihydro-1H-inden-1-one (1.92 g) was dissolved in N,N-dimethylformamide (40 ml), and then N-chlorosuccinimide (1.47 g) was added thereto at 0° C. The reaction solution was stirred for 4 hours. After cooling down the mixture to −10° C., 1,3-dichloro-5-(3,3,3-trifluoroprop-1-en-2-yl)benzene (2.91 g) and potassium hydrocarbonate (1.32 g) were added thereto. The reaction solution was stirred for 4 hours. Water and ethyl acetate were added for the extraction. The organic layer was dried over magnesium sulfate and the solvent was removed by distillation. After the purification with silica gel chromatography, the title compound was obtained (2.85 g, 63%).

1H-NMR (CDCl3) δ: ¹H-NMR (CDCl₃) δ: 2.75 (t, 2H), 3.19 (t, 2H), 3.74 (d, 1H), 4.12 (d, 1H), 7.44 (s, 1H), 7.51 (s, 2H), 7.67 (d, 1H), 7.77-7.83 (m, 2H).

### Step 5. Synthesis of 5-[5-(3,5-dichlorophenyl)-5-(trifluoromethyl)-4,5-dihydroisoxazol-3-yl]-2,3-dihydro-1H-inden-1-ol

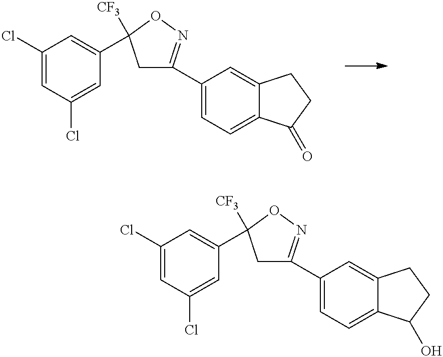

5-[5-(3,5-dichlorophenyl)-5-(trifluoromethyl)-4,5-dihydro-isoxazol-3-yl]-2,3-dihydro-1H-inden-1-ol (0.83 g) was dissolved in methanol (10 ml) and sodium borohydride (0.11 g) was added thereto at room temperature. After stirring the reaction solution overnight, the solvent was removed by distillation. Water and ethyl acetate were added for the extraction. The organic layer was dried over magnesium sulfate and the solvent was removed by distillation. The title compound was obtained as a crude product (0.77 g).

1H-NMR (CDCl3) δ: 1.90-2.03 (m, 1H), 2.48-2.60 (m, 1H), 2.48-2.60 (m, 1H), 2.77-2.90 (m, 1H), 3.00-3.13 (m, 1H), 3.70 (d, 1H), 4.09 (d, 1H), 5.27 (t, 1H), 7.40-7.59 (m, 6H).

### Step 6. Synthesis of 2-{5-[5-(3,5-dichlorophenyl)-5-(trifluoromethyl)-4,5-dihydroisoxazol-3-yl]-2,3-dihydro-1H-inden-1-yl}-1H-isoindol-1,3(2H)-dione

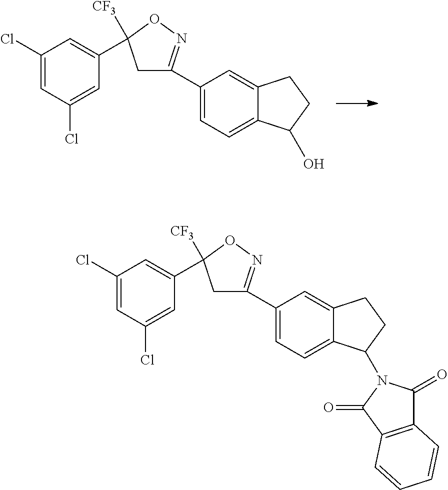

5-[5-(3,5-dichlorophenyl)-5-(trifluoromethyl)-4,5-dihydroisoxazol-3-yl]-2,3-dihydro-1H-inden-1-ol (0.80 g), phthalimide (0.31 g) and triphenylphosphine (0.71 g) were dissolved in tetrahydrofuran (10 ml), and then diethyl azodicarboxylc acid (40%/toluene, 1.32 ml) was added thereto at room temperature. The reaction solution was stirred for 3 hours. The solvent was removed by distillation. After the purification with silica gel chromatography, the title compound was obtained (0.58 g, 55%).

1H-NMR (CDCl3) δ: 2.42-2.66 (m, 2H), 2.95-3.09 (m, 1H), 3.31-3.43 (m, 1H), 3.68 (d, 1H), 4.07 (m, 1H), 5.89 (t, 1H), 7.15 (s, 1H), 7.42 (s, 1H), 7.45 (d, 1H), 7.50 (s, 2H), 7.61 (d, 1H), 7.70-7.78 (m, 2H), 7.80-7.90 (m, 2H).

### Step 7. Synthesis of 5-[5-(3,5-dichlorophenyl)-5-(trifluoromethyl)-4,5-dihydroisoxazol-3-yl]-2,3-dihydro-1H-inden-1-amine

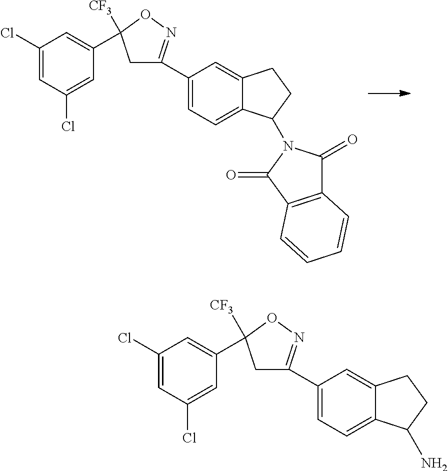

2-{5-[5-(3,5-dichlorophenyl)-5-(trifluoromethyl)-4,5-dihydro-isoxazol-3-yl]-2,3-dihydro-1H-inden-1-yl}-1H-isoindol-1,3(2H-dione (0.58 g) was dissolved in ethanol (10 ml), and then hydrazine hydrate (0.10 ml) was added thereto. The reaction solution was heated at 80° C. for 5 hours. The solvent was removed by distillation and ethyl acetate was added. Precipitates were removed by filtration, and the filtrate was concentrated. The title compound was obtained as a crude product (0.39 g).

1H-NMR (CDCl3) δ: 1.64-1.80 (m, 1H), 2.10 (bs, 2H), 2.47-2.61 (m, 1H), 2.75-2.90 (m, 1H), 2.90-3.05 (m, 1H), 3.69 (d, 1H), 4.08 (d, 1H), 4.38 (t, 1H), 7.35-7.44 (m, 2H), 7.46-7.57 (m, 4H).

### Step 8. Synthesis of N-{5-[5-(3,5-dichlorophenyl)-5-(trifluoromethyl)-4,5-dihydroisoxazol-3-yl]-2,3-dihydro-1H-inden-1-yl}acetamide

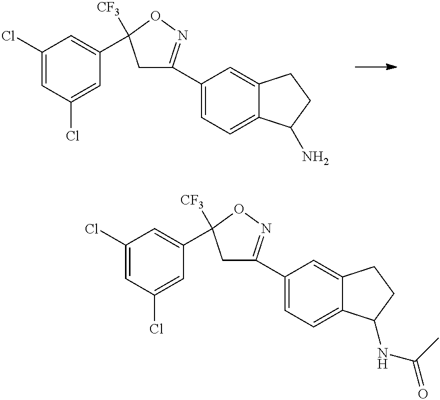

5-[5-(3,5-dichlorophenyl)-5-(trifluoromethyl)-4,5-dihydro-isoxazol-3-yl]-2,3-dihydro-1H-inden-1-amine (0.10 g) was dissolved in tetrahydrofuran (2 ml), and then anhydrous acetic acid (0.028 ml) was added thereto at room temperature. After stirring overnight, the solvent was removed by distillation. After the purification with silica gel chromatography, the title compound was obtained (0.086 g, 75%).

1H-NMR (CDCl3) δ: 1.74-1.91 (m, 1H), 2.06 (s, 3H), 2.57-2.70 (m, 1H), 2.82-3.07 (m, 2H), 3.69 (d, 1H), 4.07 (d, 1H), 5.52 (q, 1H), 5.60-5.67 (d, 1H), 7.34 (d, 1H), 7.40-7.61 (m, 5H).

### H: Synthesis of 6-[5-(3,5-dichlorophenyl)-5-(trifluoromethyl)-4,5-dihydroisoxazol-3-yl]-1,2,3,4-tetrahydronaphthalen-1-amine (No. 3-211).

### Step 1. Synthesis of 5-oxo-5,6,7,8-tetrahydronaphthalen-2-yl trifluoromethane sulfonate

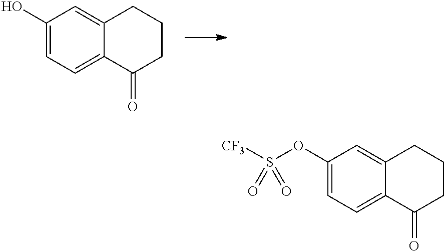

6-hydroxy-3,4-dihydro-1(2H)-naphthalenone (10.30 g) and 2,6-lutidine (14.80 ml) were dissolved in methylene chloride (150 ml), and anhydrous trifluoromethane sulfonic acid (25 g) was added thereto at 0° C. The reaction solution was stirred overnight at room temperature. Dilute hydrochloric acid was added for the extraction. The organic layer was dried over magnesium sulfate and the solvent was removed by distillation. After the purification with silica gel chromatography, the title compound was obtained (17.00 g, 91%).

1H-NMR (CDCl3) δ: 2.12-2.24 (m, 2H), 2.69 (t, 2H), 3.02 (t, 2H), 7.18-7.23 (m, 2H), 8.14 (d, 1H).

### Step 2. Synthesis of methyl (2E)-3-(5-oxo-5,6,7,8-tetrahydronaphthalen-2-yl)prop-2-enoate

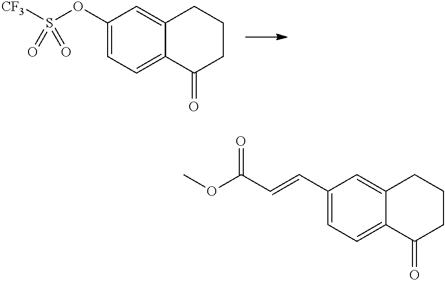

5-oxo-5,6,7,8-tetrahydronaphthalen-2-yl trifluoromethane sulfonate (7.10 g), methyl acrylate (13.0 ml), 1,3-bis(diphenylphosphino)propane (0.60 g) and triethylamine (10.1 ml) were dissolved in N,N-dimethylformamide (80 ml), and the resulting mixture was stirred under argon atmosphere for 10 min. To the reaction solution, palladium acetic acid (0.27 g) was added and the reaction solution was heated at 80° C. for 10 hours. Water and ethyl acetate were added for the extraction. The organic layer was dried over magnesium sulfate and the solvent was removed by distillation. After the purification with silica gel chromatography, the title compound was obtained (4.10 g, 74%).

1H-NMR (CDCl3) δ: 2.09-2.21 (m, 1H), 2.67 (t, 1H), 2.98 (t, 1H), 3.82 (s, 3H), 6.51 (d, 1H), 7.39 (s, 1H), 7.46 (d, 1H), 7.68 (d, 1H), 8.04 (d, 1H).

### Step 3. Synthesis of 5-oxo-5,6,7,8-tetrahydronaphthalen-2-carbaldehyde

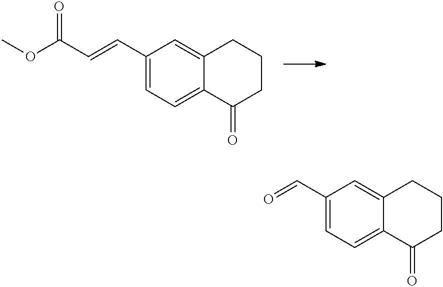

Methyl (2E)-3-(5-oxo-5,6,7,8-tetrahydronaphthalen-2-yl)prop-2-enoate was dissolved in 1,2-dichloroethane (70 ml)-water (70 ml), and sodium periodate (13.25 g) and ruthenium trichloride hydrate (0.18 g) were added thereto at room temperature. The reaction solution was stirred for 4 hours. Sodium thiosulfate and ethyl acetate were added for the extraction. The organic layer was dried over magnesium sulfate and the solvent was removed by distillation. After the purification with silica gel chromatography, the title compound was obtained (1.92 g, 50%).

1H-NMR (CDCl3) δ: 2.10-2.25 (m, 2H), 2.72 (t, 2H), 3.07 (t, 2H), 7.72-7.83 (m, 2H), 8.18 (d, 1H), 10.08 (s, 1H).

### Step 4. Synthesis of 6-[(E)-(hydroxyimino)methyl]-3,4-dihydronaphthalen-1(2H)-one

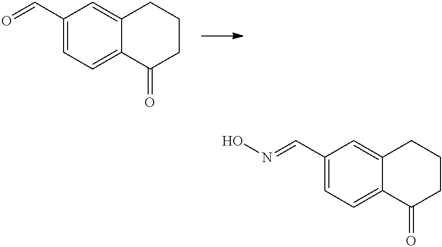

1-oxo-2,3-dihydro-1H-inden-5-carbaldehyde (2.46 g) and sodium hydrocarbonate (1.42 g) were suspended in ethanol (70 ml), and hydroxyamine hydrochloride (0.98 g) was added thereto at 0° C. After stirring for 1 hour, the solvent was removed by distillation. Water and ethyl acetate were added for the extraction. The organic layer was dried over magnesium sulfate and the solvent was removed by distillation. The title compound was obtained as a crude product (2.60 g).

1H-NMR (CDCl3) δ: 2.10-2.21 (m, 2H), 2.68 (t, 2H), 2.99 (t, 2H), 7.47 (s, 1H), 7.52 (d, 1H), 7.75 (bs, 1H), 8.05 (d, 1H), 8.14 (s, 1H).

### Step 5. Synthesis of 6-[5-(3,5-dichlorophenyl)-5-(trifluoromethyl)-4,5-dihydroisoxazol-3-yl]-3,4-dihydronaphthalen-1(2H)-one

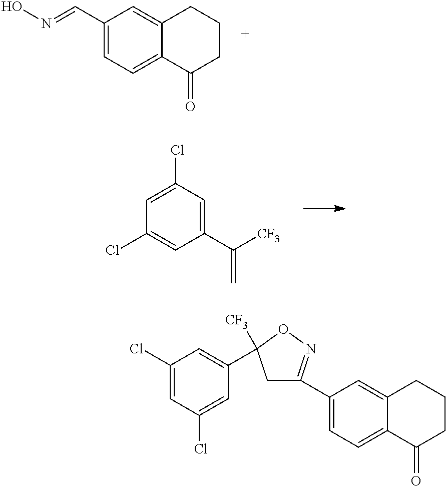

6-[(E)-(hydroxyimino)methyl]-3,4-dihydronaphthalen-1(2H)-one (2.67 g) was dissolved in N,N-dimethylformamide (50 ml), and then N-chlorosuccinimide (1.89 g) was added thereto at 0° C. The reaction solution was stirred for 4 hours. While keeping the temperature of the reaction solution at 0° C., 1,3-dichloro-5-(3,3,3-trifluoroprop-1-en-2-yl)benzene (3.74 g) and potassium hydrocarbonate (1.70 g) were added thereto. The reaction solution was stirred for 3 hours. Water and ethyl acetate were added for the extraction. The organic layer was dried over magnesium sulfate and the solvent was removed by distillation. After the purification with silica gel chromatography, the title compound was obtained (3.60 g, 60%).

1H-NMR (CDCl3) δ: 2.11-2.22 (m, 2H), 2.69 (t, 2H), 3.00 (t, 2H), 3.71 (d, 1H), 4.10 (d, 1H), 7.41-7.59 (m, 5H), 8.08 (d, 1H).

### Step 6. Synthesis of 6-[5-(3,5-dichlorophenyl)-5-(trifluoromethyl)-4,5-dihydroisoxazol-3-yl]-1,2,3,4-tetrahydronaphthalen-1-amine

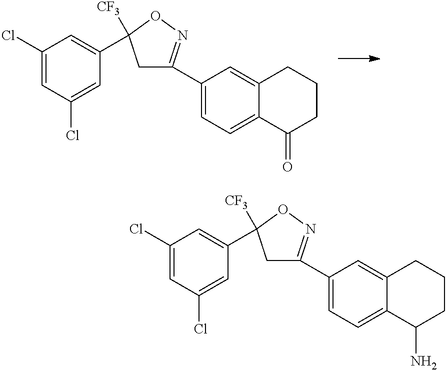

6-[5-(3,5-dichlorophenyl)-5-(trifluoromethyl)-4,5-dihydro-isoxazol-3-yl]-3,4-dihydronaphthalen-1(2H)-one (1.56 g) and ammonium acetate (4.21 g) were dissolved in methanol (40 ml), and then sodium cyanotrihydro borate (0.69 g) was added thereto at room temperature. The reaction solution was heated at 70° C. for 8 hours. The solvent was removed by distillation, and water and tert-butylmethyl ether were added thereto. The aqueous layer was separated off and the organic layer was extracted with concentrated hydrochloric acid. The acidic aqueous layer was neutralized with sodium carbonate and extracted with tert-butylmethyl ether. The organic layer was dried over magnesium sulfate and the solvent was removed by distillation. The title compound was obtained as a crude product (1.15 g).

1H-NMR (CDCl3) δ: 1.47-2.11 (m, 6H), 2.68-2.90 (m, 2H), 3.68 (d, 1H), 3.98 (t, 1H), 4.07 (d, 1H), 7.37 (s, 1H), 7.42 (t, 1H), 7.48 (s, 2H), 7.51 (s, 2H).

### I: Synthesis of 5-[5-(3,5-dichlorophenyl)-5-(trifluoromethyl)-4,5-dihydroisoxazol-3-yl]-N-ethyl-2,3-dihydro-1H-indol-1-carboxamide (No. 3-638)

### Step 1. Synthesis of tert-butyl 5-formyl-1H-indol-1-carboxyate

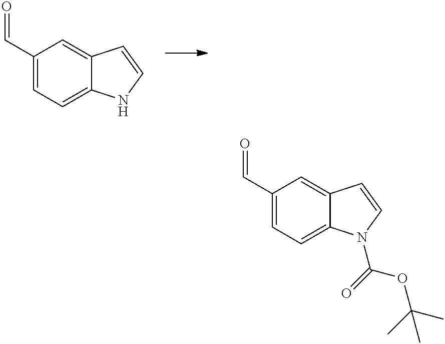

Indol-5-carboxyaldehyde (8.00 g) and 4-dimethylaminopyridine (0.67 g) were dissolved in acetonitrile (200 ml), and di-tert-butyl bicarbonate (15.6 g) was added thereto at room temperature. After stirring overnight, the solvent was removed by distillation. After the dilution with ethyl acetate, the mixture was washed with dilute hydrochloric acid and an aqueous solution of sodium carbonate. After drying over magnesium sulfate, the solvent was removed by distillation. The title compound was obtained as a crude product (13.5 g).

1H-NMR (CDCl3) δ: 1.69 (s, 9H), 6.69 (d, 1H), 7.69 (d, 1H), 7.86 (d, 1H), 8.10 (s, 1H), 8.29 (d, 1H), 10.07 (s, 1H).

### Step 2. Synthesis of tert-butyl 5-(hydroxymethyl)-2,3-dihydro-1H-indol-1-carboxyate

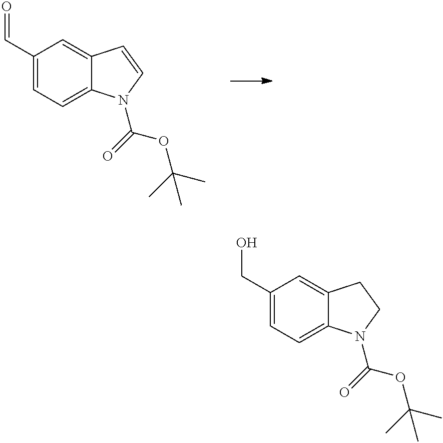

Tert-butyl 5-formyl-1H-indol-1-carboxyate (13.5 g) and triethylamine (7.67 ml) were dissolved in ethanol (150 ml), and then palladium-activated charcoal (10%, 1.33 g) was added thereto at room temperature. Hydrogen gas was introduced thereto using a balloon and the mixture was stirred for 4 days. The catalyst was removed by filtration, and the filtrate was concentrated under reduced pressure. After the purification with silica gel chromatography, the title compound was obtained (2.91 g, 52%, 21%).

1H-NMR (CDCl3) δ: 1.52-1.62 (m, 10H), 3.08 (t, 2H), 3.98 (t, 2H), 4.61 (d, 2H), 7.10-7.25 (m, 2H), 7.66-7.92 (m, 1H).

### Step 3. Synthesis of tert-butyl 5-formyl-2,3-dihydro-1H-indol-1-carboxyate

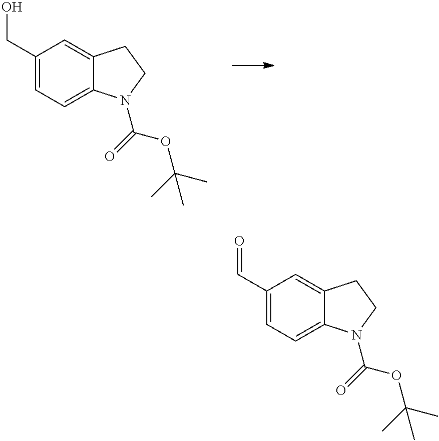

Tert-butyl 5-(hydroxymethyl)-2,3-dihydro-1H-indol-1-carboxyate (2.91 g) was dissolved in methylene chloride (50 ml), and then activated manganese dioxide (10.1 g) was added thereto at room temperature. The mixture was stirred overnight. The solids were removed by filtration, and the filtrate was concentrated under reduced pressure. As a result, the title compound was obtained as a crude product (2.73 g).

1H-NMR (CDCl3) δ: 1.58 (s, 9H), 3.15 (t, 2H), 4.05 (t, 2H), 7.62-7.73 (m, 2H), 7.80-8.01 (m, 1H), 9.86 (s, 1H).

### Step 4. Synthesis of tert-butyl 5-[(E)-(hydroxyimino)methyl]-2,3-dihydro-1H-indol-1-carboxyate

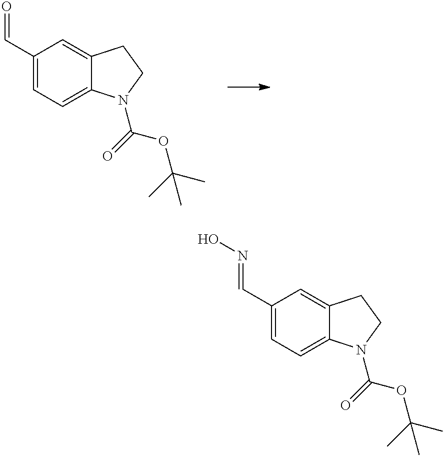

Tert-butyl 5-formyl-2,3-dihydro-1H-indol-1-carboxyate (2.73 g) and sodium hydrocarbonate (1.39 g) were suspended in ethanol, and then hydroxyamine hydrochloride salt (0.92 g) was added thereto at room temperature. The reaction solution was stirred overnight and the solvent was removed by distillation. Water and ethyl acetate were added for the extraction. The organic layer was dried over magnesium sulfate and the solvent was removed by distillation. As a result, the title compound was obtained as a crude product (2.50 g).

1H-NMR (CDCl3) δ: 1.57 (s, 9H), 3.10 (t, 2H), 4.00 (t, 2H), 7.24-7.48 (m, 2H), 7.62-7.92 (m, 1H), 8.07 (s, 1H).

### Step 5. Synthesis of tert-butyl 5-[5-(3,5-dichlorophenyl)-5-(trifluoromethyl)-4,5-dihydro-isoxazol-3-yl]-2,3-dihydro-1H-indol-1-carboxyate

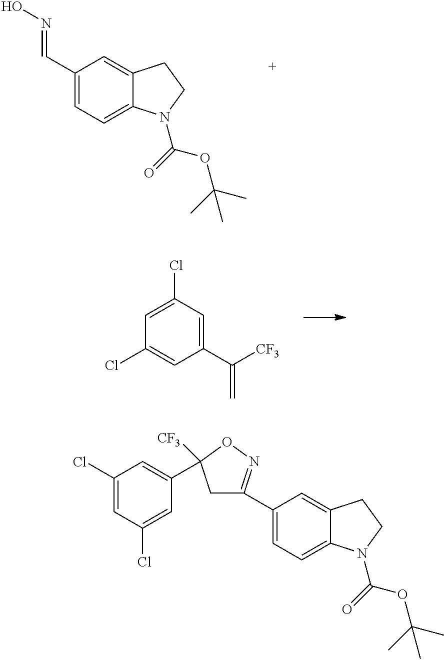

Tert-butyl 5-[(E)-(hydroxyimino)methyl]-2,3-dihydro-1H-indol-1-carboxyate (2.50 g) was dissolved in N,N-dimethylformamide (50 ml), and then N-chlorosuccinimide (1.27 g) was added thereto at room temperature. The reaction solution was then stirred for 4 hours. After cooling the solution to 0° C., 1,3-dichloro-5-(3,3,3-trifluoroprop-1-ene-2-yl)benzene (2.53 g) and potassium hydrocarbonate (1.15 g) were added thereto. The reaction solution was stirred overnight at room temperature. Water and ethyl acetate were added for the extraction. The organic layer was dried over magnesium sulfate and the solvent was removed by distillation. As a result, the title compound was obtained as a crude product (4.78 g).

1H-NMR (CDCl3) δ: 1.57 (s, 9H), 3.11 (t, 2H), 3.67 (d, 1H), 3.94-4.06 (m, 3H), 7.30-7.59 (m, 6H).

### Step 6. Synthesis of 5-[5-(3,5-dichlorophenyl)-5-(trifluoromethyl)-4,5-dihydroisoxazol-yl]-2,3-dihydro-1H-indole

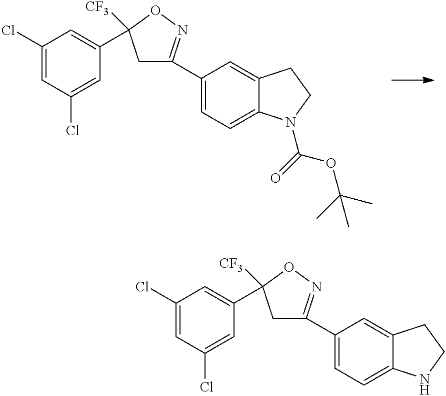

Tert-butyl 5-[5-(3,5-dichlorophenyl)-5-(trifluoromethyl)-4,5-dihydro-isoxazol-3-yl]-2,3-dihydro-1H-indol-1-carboxyate (4.78 g), concentrated hydrochloric acid (20 ml) and ethanol (75 ml) were mixed and the resulting mixture was heated at 80° C. for 8 hours. Solvent was removed by distillation, and water and ethyl acetate were added. The resulting mixture was neutralized by adding sodium carbonate, and then extracted. The organic layer was dried over magnesium sulfate and the solvent was removed by distillation. After the purification with silica gel chromatography, the title compound was obtained (3.37 g, 88%).

1H-NMR (CDCl3) δ: 1.58 (bs, 1H), 3.05 (t, 2H), 3.57-3.69 (m, 3H), 4.04 (d, 1H), 6.56 (d, 1H), 7.23 (d, 1H), 7.40 (s, 1H), 7.47 (s, 1H), 7.51 (s, 2H).

### Step 7. Synthesis of 5-[5-(3,5-dichlorophenyl)-5-(trifluoromethyl)-4,5-dihydroisoxazol-3-yl]-N-ethyl-2,3-dihydro-1H-indol-1-carboxamide

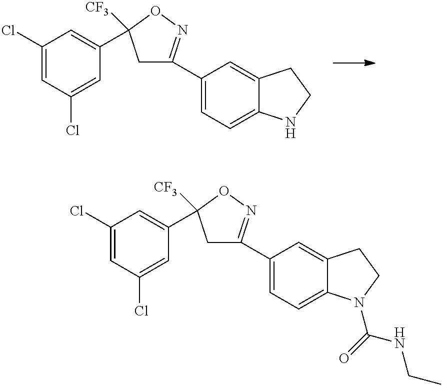

5-[5-(3,5-dichlorophenyl)-5-(trifluoromethyl)-4,5-dihydro-isoxazol-3-yl]-2,3-dihydro-1H-indole (0.12 g) and triethylamine (0.042 ml) were dissolved in tetrahydrofuran (2 ml), and ethyl isocyanate (0.047 ml) was added thereto at room temperature. After stirring the mixture overnight, the solvent was removed by distillation. After the purification with silica gel chromatography, the title compound was obtained (0.104 g, 74%).

1H-NMR (CDCl3) δ: 1.22 (t, 3H), 3.20 (t, 2H), 3.39 (quintet, 2H), 3.68 (d, 1H), 3.93 (t, 2H), 4.07 (d, 1H), 4.57-4.66 (m, 1H), 7.31 (d, 1H), 7.41 (s, 1H), 7.51 (s, 2H), 7.58 (s, 1H), 7.98 (d, 1H).

### J: Synthesis of 6-[5-(3,5-dichlorophenyl)-5-(trifluoromethyl)-4,5-dihydroisoxazol-3-yl]-N-ethyl-3,4-dihydroquinolin-1(2H)-carboxamide (No. 3-638).

### Step 1. Synthesis of (E)-N-hydroxyl-(quinolin-6-yl)methaneimine

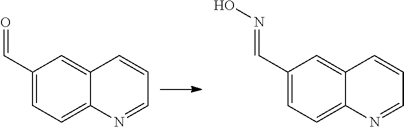

Quinolin-6-carbaldehyde (1.01 g) and triethylamine (1.34 ml) were dissolved in ethanol (70 ml), and hydroxyamine hydrochloride (0.54 g) was added thereto at room temperature. After stirring the mixture overnight, the solvent was removed by distillation. Water and ethyl acetate were added thereto for extraction. The organic layer was dried over magnesium sulfate and the solvent was removed by distillation. The title compound was obtained as a crude product (1.00 g).

1H-NMR (CDCl3) δ: 1.57 (bs, 1H), 7.41-7.49 (m, 1H), 7.89 (s, 1H), 8.06-8.23 (m, 3H), 8.32 (s, 1H), 8.90-8.97 (m, 1H).

### Step 2. Synthesis of 6-[5-(3,5-dichlorophenyl)-5-(trifluoromethyl)-4,5-dihydroisoxazol-3-yl]quinoline

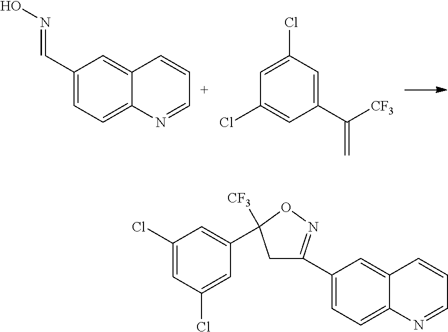

(E)-N-hydroxyl-(quinolin-6-yl)methaneimine (0.98 g) was dissolved in N,N-dimethylformamide (50 ml), and then N-chlorosuccinimide (0.83 g) was added thereto at room temperature. The reaction solution was stirred for 1 hour at 50° C. After cooling the reaction solution to 0° C., 1,3-dichloro-5-(3,3,3-trifluoroprop-1-ene-2-yl)benzene (1.50 g) and potassium hydrocarbonate (0.68 g) were added thereto. The reaction solution was stirred overnight at room temperature. Water and ethyl acetate were added for the extraction. The organic layer was dried over magnesium sulfate and the solvent was removed by distillation. After washing with a small amount of hexane for purification, the title compound was obtained (0.88 g, 38%).

1H-NMR (CDCl3) δ: 3.83 (d, 1H), 4.23 (d, 1H), 7.41-7.59 (m, 4H), 7.95 (s, 1H), 8.13-8.22 (m, 3H), 8.95-9.00 (m, 1H).

### Step 3. Synthesis of 6-[5-(3,5-dichlorophenyl)-5-(trifluoromethyl)-4,5-dihydroisoxazol-3-yl]-1,2,3,4-tetrahydroquinoline

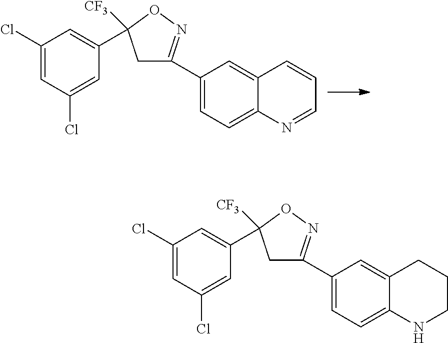

6-[5-(3,5-dichlorophenyl)-5-(trifluoromethyl)-4,5-dihydroisoxazol-3-yl]quinoline (0.76 g) and sodium cyanotrihydroborate (0.23 g) were dissolved in methanol (20 ml), and then boron trifluoro diethyl ether complex (0.58 ml) was added thereto. The reaction solution was refluxed for 6 hours under heating. The solvent was removed by distillation. After the purification with silica gel chromatography, the title compound was obtained (0.52 g).

¹H-NMR (CDCl3) δ: 1.91-1.93 (2H, m), 2.73-2.75 (2H, m), 3.33-3.35 (2H, m), 3.62 (1H, d), 4.02 (1H), 4.21 (1H, s), 6.41 (1H, d), 7.24-7.25 (2H, m), 7.39 (1H, t), 7.51 (2H, s).

### Step 4. Synthesis of 6-[5-(3,5-dichlorophenyl)-5-(trifluoromethyl)-4,5-dihydroisoxazol-3-yl]-N-ethyl-3,4-dihydroquinolin-1(2H)-carboxamide

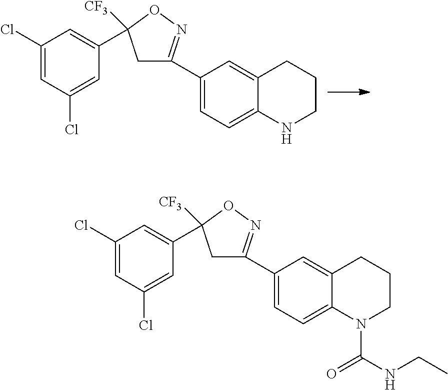

To the ethyl acetate solution (5 ml) of 6-[5-(3,5-dichlorophenyl)-5-(trifluoromethyl)-4,5-dihydroisoxazol-3-yl]-1,2,3,4-tetrahydroquinoline (0.163 g), diisopropylethylamine (82 mg) and bis(trichloromethyl)carbonate (64 mg) were added under ice cooling. The reaction solution was stirred for 90 min under ice cooling. After adding an aqueous solution of 70% ethylamine (0.16 ml) to the reaction solution under ice cooling, the reaction solution was stirred at room temperature for 2 hours. Ethyl acetate and water were added to the reaction solution to separate the organic layer, which was then washed with brine and dried over magnesium sulfate. After the filtration, the solution was concentrated under reduced pressure. Residues were purified by silica gel chromatography using a solvent of n-hexane/ethyl acetate (1:2) to obtain the desired compound of 6-[5-(3,5-dichlorophenyl)-5-(trifluoromethyl)-4,5-dihydro-isoxazol-3-yl]-N-ethyl-3,4-dihydroquinolin-1(2H)-carboxamide (150 mg).

1H-NMR (CDCl3) δ: 1.15 (3H, t), 1.89-1.98 (2H, m), 2.76 (2H, t), 3.28-3.38 (2H, m), 3.64-3.75 (3H, m), 4.06 (1H, d), 5.02 (1H, t), 7.44-7.48 (7H, m).

### K: Synthesis of 1-{5-[3-(3,5-dichlorophenyl)-3-(trifluoromethyl)-3,4-dihydro-2H-pyrrol-5-yl]-2,3-dihydro-1H-inden-1-yl}-3-ethylurea (No. 3-112)

### Step 1. Synthesis of 1-oxo-2,3-dihydro-1H-inden-5-carbonitrile

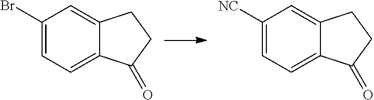

Under the argon atmosphere, N,N-dimethylformamide solution (50 ml) of 5-bromo-1-indane (5 g), zinc cyanide (1.9 g), and palladium tetrakistriphenylphosphine (2.7 g) was stirred for 1 hour at 85° C. After cooling, the reaction solution was diluted with tert-butylmethylether and washed twice with water. The organic layer was dried over anhydrous magnesium sulfate and then the solvent was removed by distillation under reduced pressure. Thus obtained crystals were washed with tert-butylmethylether to obtain 1-oxo-2,3-dihydro-1H-inden-5-carbonitrile (3.0 g).

1H-NMR (CDCl₃) δ: 2.76-2.80 (2H, m), 3.21-3.24 (2H, m), 7.65-7.67 (1H, m), 7.82-7.85 (2H, m).

### Step 2. Synthesis of 1-oxo-2,3-dihydro-1H-inden-5-carboxylc acid

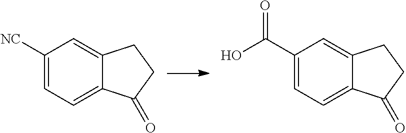

1-oxo-2,3-dihydro-1H-inden-5-carbonitrile (1.0 g) was suspended in a solution of concentrated hydrochloric acid (10 ml) and acetic acid (20 ml), and stirred at 120° C. for 16 hours. After cooling, the reaction solution was concentrated under reduced pressure, tert-butyl methyl ether and water was added and then stirred. The organic layer was dried over anhydrous magnesium sulfate. After removing the solvent by distillation under reduced pressure, 1-oxo-2,3-dihydro-1H-inden-5-carboxylc acid (0.7 g) was obtained.

1H-NMR (acetone-d₆) δ: 2.69-2.75 (3H, m), 3.23-3.25 (3H, m), 7.74 (1H, d), 8.04 (1H, d), 8.19 (1H, s).

### Step 3. Synthesis of 1-oxo-N-[(trimethylsilylmethyl]-2,3-dihydro-1Hinden-5-carboxyamide

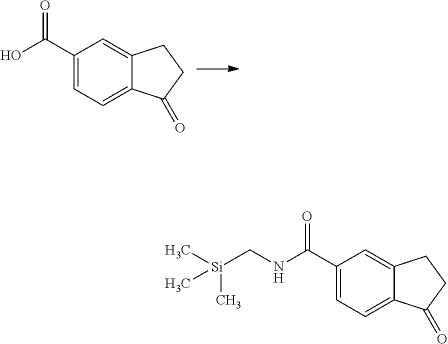

1-oxo-2,3-dihydro-1H-inden-5-carboxylc acid (0.1 g), (trimethylsilyl)methylamine (0.06 g), N,N-dimethylaminopyridine (0.01 g) and N-(3-dimethylaminopropyl)-N′-ethylcarbodiimide hydrochloride (0.12 g) were dissolved in methylene chloride (5 ml), and the resulting mixture was stirred for 5 hours at room temperature. After adding water, the reaction solution was stirred and the organic layer was dried over anhydrous magnesium sulfate. After removing the solvent by distillation under reduced pressure, and the resulting residues were purified with silica gel chromatography, 1-oxo-N-[(trimethylsilylmethyl]-2,3-dihydro-1Hinden-5-carboxyamide was obtained (0.11 g).

¹H-NMR (CDCl₃) δ: 0.15 (9H, s), 2.72-2.76 (2H, m), 2.99 (2H, d, J=5.9 Hz), 3.18-3.20 (2H, m), 6.05 (1H, br s), 7.66 (1H, d), 7.79 (1H, d), 7.87 (1H, s).

### Step 4. Synthesis of tert-butyl(5-{[(trimethylsilyl)methyl]carbamoyl}-2,3-dihydro-1H-inden-1-yl)carbamate

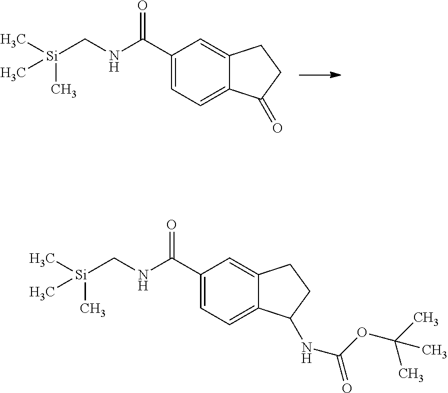

1-oxo-N-[(trimethylsilylmethyl]-2,3-dihydro-1Hinden-5-carboxyamide (1.0 g), ammonium acetate (3.2 g) and sodium cyanoborohydride (0.4 g) were dissolved in methanol (30 ml), refluxed for 6 hours under the argon atmosphere, and stirred further at room temperature for 5 hours. The solvent was removed by distillation under reduced pressure, and tert-butyl methyl ether and water were added to the residues and stirred. The organic layer was dried over anhydrous magnesium sulfate and the solvent was removed by distillation under reduced pressure. The residues were dissolved in toluene (30 ml), di-tert-butyl bicarbonate (1.0 g) was added thereto, and the resulting mixture was heated under stirring for 30 min at 100° C. After cooling, the solvent was removed by distillation and the residues were purified by silica gel chromatography to obtain tert-butyl(5-{[(trimethylsilyl)methyl]carbamoyl}-2,3-dihydro-1H-inden-1-yl)carbamate (0.25 g).

¹H-NMR (CDCl₃) δ: 0.13 (9H,$), 1.49 (9H, s), 1.78-1.84 (1H, m), 2.56-2.66 (1H, m), 2.85-2.98 (4H, m), 4.74-4.77 (1H, m), 5.18-5.21 (1H, m), 5.93-5.96 (1H, m), 7.35 (1H, d), 7.55-7.58 (2H, m).

### Step 5. Synthesis of tert-butyl(5-{[(trimethylsilyl)methyl]carbamothioyl}-2,3-dihydro-1H-inden-1-yl)carbamate

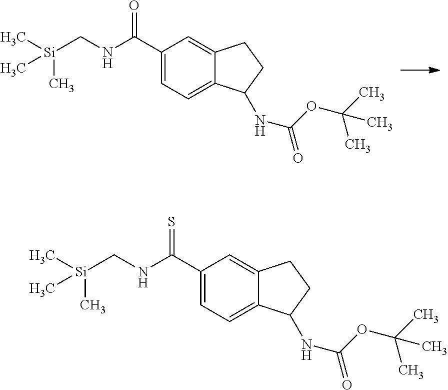

Tert-butyl(5-{[(trimethylsilyl)methyl]carbamoyl}-2,3-dihydro-1H-inden-1-yl)carbamate (0.25 g) and Lawesson's reagent (0.2 g) were suspended in toluene (10 ml), and the resulting mixture was refluxed under heating for 1 hour. After cooling down to room temperature, the reaction solution was washed with water and saturated brine. The organic layer was dried over anhydrous magnesium sulfate. After removing the solvent under reduced pressure, and the residues were purified by silica gel chromatography, tert-butyl(5-{[(trimethylsilyl)methyl]carbamothioyl}-2,3-dihydro-1H-inden-1-yl)carbamate was obtained (0.25 g).

¹H-NMR (CDCl₃) δ: 0.18 (9H, s), 1.46 (9H, s), 1.69-1.77 (1H, m), 2.46-2.56 (1H, m), 2.71-2.97 (2H, m), 3.52 (2H, d), 4.76-4.79 (1H, m), 4.98-5.01 (1H, m), 7.23 (1H, d), 7.45 (1H, d), 7.57 (1H, s), 7.96 (1H, br s).

### Step 6. Synthesis of methyl 1-[(tert-butoxycarbonyl)amino]-N-[(trimethylsilyl)methyl]-2,3-dihydro-1H-inden-5-carboimide thioate

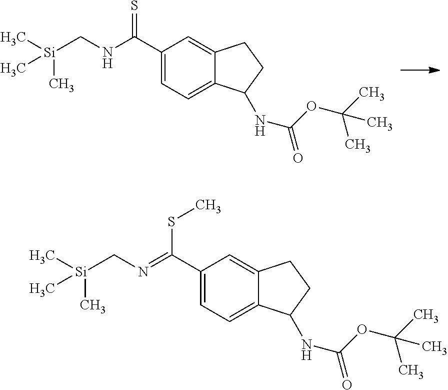

Tetrahydrofuran (10 ml) solution of methyl iodide (0.1 g), potassium tert-butoxide (0.09 g) and tert-butyl(5-{[(trimethylsilyl)methyl]carbamothioyl}-2,3-dihydro-1H-inden-1-yl)carbamate (0.25 g) was stirred for 2 hours at room temperature. The reaction solution was diluted by adding t-butyl methyl ether, and then washed with water and saturated brine. The organic layer was dried over anhydrous magnesium sulfate. After removing the solvent by distillation, residues were simply purified by silica gel chromatography to obtain methyl 1-[(tert-butoxycarbonyl)amino-]-N-[(trimethylsilyl)methyl]-2,3-dihydro-1H-inden-5-carboimide thioate (0.25 g).

### Step 7. Synthesis of tert-butyl {5-[3-(3,5-dichlorophenyl)-3-(trifluoromethyl)-3,4-dihydro-2H-pyrrol-5-yl]-2,3-dihydro-1H-inden-1-yl}carbamate

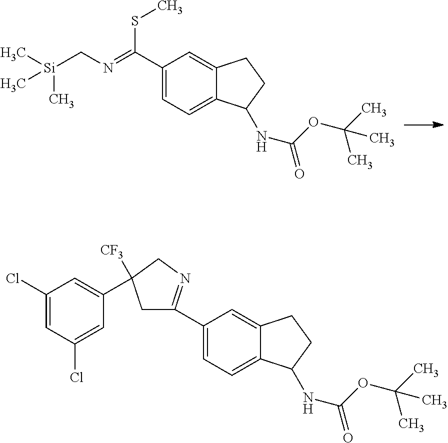

Under the argon atmosphere, tetrahydrofuran solution (10 ml) of methyl 1-[(tert-butoxycarbonyl)amino]-N-[(trimethylsilyl)methyl]-2,3-dihydro-1H-inden-5-carboimide thioate (0.25 g) and 1,3-dichloro-5-(3,3,3-trifluoropro-1-pene-2-yl)benzene (0.15 g) was cooled to −5° C., and then 1M tetrahydrofuran solution of tetrabutylammonium fluoride (0.2 ml) was slowly added dropwise thereto. The reaction solution was stirred at room temperature for 20 hours, and then diluted with tert-butyl methyl ether and washed with water and saturated brine. The organic layer was dried over anhydrous magnesium sulfate and the solvent was removed by distillation under reduced pressure. The residues were purified by silica gel chromatography to obtain tert-butyl {5-[3-(3,5-dichlorophenyl)-3-(trifluoromethyl)-3,4-dihydro-2H-pyrrol-5-yl]-2,3-dihydro-1H-inden-1-yl}carbamate (0.23 g).

¹H-NMR (CDCl₃) δ: 1.49 (9H, s), 1.78-1.88 (1H, m), 2.59-2.62 (1H, m), 2.80-3.02 (2H, m), 3.44 (1H, d), 3.79 (1H, d), 4.41 (1H, d), 4.83-4.89 (2H, m), 5.19-5.22 (1H, m), 7.26-7.29 (2H, m), 7.36-7.39 (2H, m), 7.66-7.73 (2H, m).

### Step 8. Synthesis of 5-[3-(3,5-dichlorophenyl)-3-(trifluoromethyl)-3,4-dihydro-2H-pyrrol-5-yl]-2,3-dihydro-1H-inden-1-amine

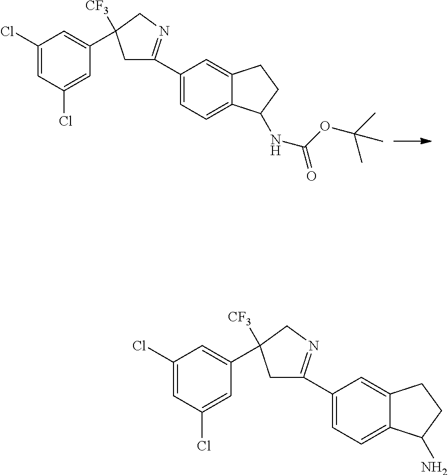

To the methylenechloride solution (10 ml) of tert-butyl {5-[3-(3,5-dichlorophenyl)-3-(trifluoromethyl)-3,4-dihydro-2H-pyrrol-5-yl]-2,3-dihydro-1H-inden-1-yl}carbamate (0.23 g), trifluoroacetic acid (0.5 g) was added and then the resulting mixture was stirred at room temperature for 2 hours. Under reduced pressure, the solvent was removed by distillation, and tert-butyl methyl ether and a saturated aqueous solution of sodium hydrocarbonate were added to the residues and stirred. The organic layer was dried over anhydrous magnesium sulfate and the solvent was removed by distillation under reduced pressure to obtain 5-[3-(3,5-dichlorophenyl)-3-(trifluoromethyl)-3,4-dihydro-2H-pyrrol-5-yl]-2,3-dihydro-1H-inden-1-amine (0.17 g).

¹H-NMR (CDCl₃) δ: 1.85-1.95 (1H, m), 2.51-2.55 (1H, m), 2.82-3.46 (5H, m), 3.77 (1H, d), 4.36-4.52 (2H, m), 4.80-4.85 (1H, m), 7.24-7.28 (2H, m), 7.36-7.37 (1H, m), 7.45 (1H, d, J=7.9 Hz), 7.69-7.72 (2H, m).

### Step 9. Synthesis of 1-{5-[3-(3,5-dichlorophenyl)-3-(trifluoromethyl)-3,4-dihydro-2H-pyrrol-5-yl]-2,3-dihydro-1H-inden-1-yl}-3-ethylurea

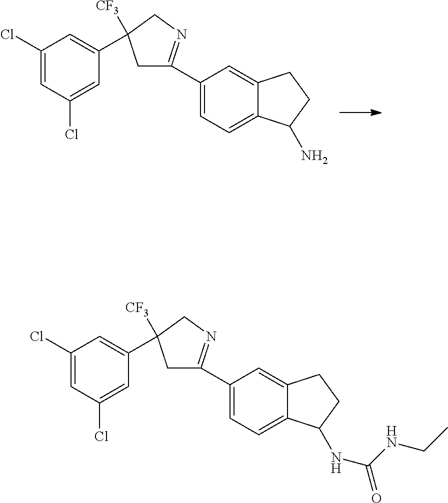

After stirring the tetrahydrofuran solution (5 ml) of 5-[3-(3,5-dichlorophenyl)-3-(trifluoromethyl)-3,4-dihydro-2H-pyrrol-5-yl]-2,3-dihydro-1H-inden-1-amine (0.09 g) and ethyl isocyanate (0.02 g) for 16 hours, the solvent was removed by distillation. With the purification based on silica gel chromatography, 1-{5-[3-(3,5-dichlorophenyl)-3-(trifluoromethyl)-3,4-dihydro-2H-pyrrol-5-yl]-2,3-dihydro-1H-inden-1-yl}-3-ethylurea was obtained (0.06 g).

¹H-NMR (CDCl₃) δ: 1.11 (3H, t), 1.65-1.82 (1H, m), 2.51-2.56 (1H, m), 2.77-2.89 (2H, m), 3.17-3.22 (2H, m), 3.42 (1H, d), 3.77 (1H, d), 4.40 (1H, d), 4.81-4.86 (2H, m), 4.98 (1H, d), 5.25-5.28 (1H, m), 7.27-7.37 (4H, m), 7.62-7.68 (2H, m).

### L: Synthesis of 6-[5-(3,5-dichlorophenyl)-5-(trifluoromethyl)-4,5-dihydroisoxazol quinolin-2-carbonitrile (No. 1-255).

### Step 1. Synthesis of 6-[5-(3,5-dichlorophenyl)-5-(trifluoromethyl)-4,5-dihydroisoxazol-3-yl]quinoline 1-oxide

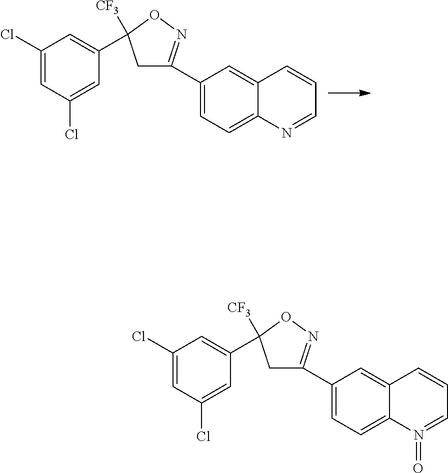

6-[5-(3,5-dichlorophenyl)-5-(trifluoromethyl)-4,5-dihydroisoxazol-3-yl]quinoline (0.30 g) was dissolved in methylene chloride (15 ml), and then 3-chloroperbenzoic acid (0.21 g) was added thereto at room temperature. After stirring the mixture for 5 hours, an aqueous solution of sodium thiosulfate was added thereto. The organic layer was separated and washed with an aqueous solution of sodium carbonate. After drying over magnesium sulfate, the title compound was obtained as a crude product (0.24 g).

1H-NMR (CDCl3) δ: 3.82 (1H, d), 4.21 (1H, d), 7.37 (1H, dd), 7.44-7.45 (1H, m), 7.53-7.54 (2H, m), 7.74 (1H, d), 8.02-8.02 (1H, m), 8.15 (1H, dd), 8.55 (1H, d), 8.79 (1H, d).

### Step 2. Synthesis of 6-[5-(3,5-dichlorophenyl)-5-(trifluoromethyl)-4,5-dihydroisoxazol-3-yl]quinolin-2-carbonitrile

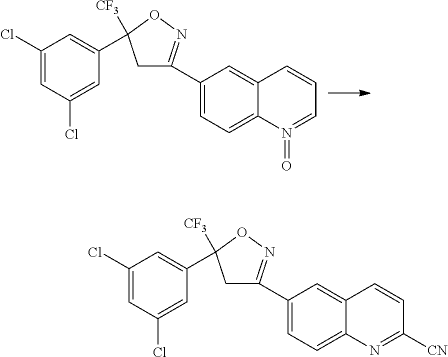

Trimethylsilylcyanide (0.30 g) and triethylamine (0.20 g) were added to acetonitrile (10 ml) solution of 6-[5-(3,5-dichlorophenyl)-5-(trifluoromethyl)-4,5-dihydroisoxazol-3-yl]quinoline 1-oxide, and then the reaction solution was refluxed for 6 hours. After adding ethyl acetate and water to the reaction solution, the organic layer was separated and washed with brine. The organic layer was dried over magnesium sulfate. After the filtration, it was concentrated under reduced pressure. The residues were purified by silica gel chromatography using the solvent of n-hexane/ethyl acetate (3:1) to obtain the title compound of 6-[5-(3,5-dichlorophenyl)-5-(trifluoromethyl)-4,5-dihydroisoxazol-3-yl]quinolin-2-carbonitrile (0.30 g).

¹H-NMR (CDCl₃) δ: 3.84 (1H, d), 4.22 (1H, d), 7.44 (1H, t), 7.54-7.54 (2H, m), 7.77 (1H, d), 8.00 (1H, d), 8.21 (1H, d Hz), 8.29-8.33 (21-I, m).

### M: Synthesis of 2-chloro-6-[5-(3,5-dichlorophenyl)-5-(trifluoromethyl)-4,5-dihydroisoxazol-3-yl]quinoline (No. 3-777) and 4-chloro-6-[5-(3,5-dichlorophenyl)-5-(trifluoromethyl)-4,5-dihydroisoxazol-3-yl]quinoline

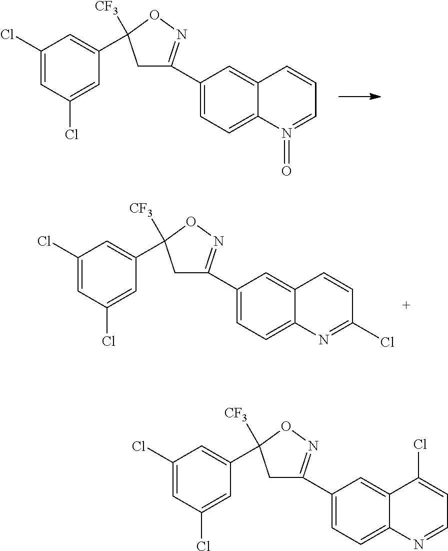

6-[5-(3,5-dichlorophenyl)-5-(trifluoromethyl)-4,5-dihydroisoxazol-3-yl]quinoline 1-oxide (0.24 g) was dissolved in phosphorus oxychloride (0.60 ml) at room temperature, and stirred for 9 hours. Ethyl acetate and water were added to the reaction solution for extraction. The organic layer was dried over magnesium sulfate, and the solvent was removed by distillation. With the purification based on silica gel chromatography, 4-chloro-6-[5-(3,5-dichlorophenyl)-5-(trifluoromethyl)-4,5-dihydroisoxazol-3-yl]quinoline was obtained (0.083 g, yield 22%), and also 2-chloro-6-[5-(3,5-dichlorophenyl)-5-(trifluoromethyl)-4,5-dihydroisoxazol-3-yl]quinoline was obtained (0.12 g, yield 30%).

1H-NMR (CDCl3) δ: 3.81 (d, 1H), 4.20 (d, 1H), 7.40-7.49 (m, 2H), 7.54 (s, 2H), 7.96 (s, 1H), 8.01-8.22 (m, 3H) of 2-chloro-6-[5-(3,5-dichlorophenyl)-5-(trifluoromethyl)-4,5-dihydro-isoxazol-3-yl]quinoline.

1H-NMR (CDCl3) δ: 3.86 (d, 1H), 4.25 (d, 1H), 7.45 (s, 1H), 7.53-7.60 (m, 3H) 8.16 (d, 1H), 8.24-8.31 (m, 2H), 8.84 (d, 1H) of 4-chloro-6-[5-(3,5-dichlorophenyl)-5-(trifluoromethyl)-4,5-dihydroisoxazol-3-yl]quinoline.

The compounds of the present invention as well as useful intermediates for the manufacturing of the compounds are described in the following tables. In the tables, abbreviations are as follows. Pr: propyl, Bu: butyl, Ph: phenyl, py: pyridyl, n-: normal, iso-: iso, tert-: tertiary, cyc-: cyclo, dio: dioxolan, pyrim: pyrimidine, pyra: pyrazole, tria: triazole, thia: thiazole. In addition, symbol (−) described in columns for W1 to W4 indicates a single bond, ie. that W is omitted.

TABLE 1

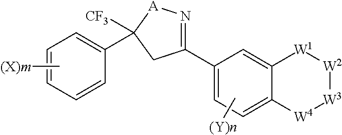

| Example | (X)m | A | (Y)n | W¹ | W² | W³ | W⁴ |
|---|---|---|---|---|---|---|---|
| 1-1 | 3,5-F2 | O | H | — | CH2 | O | C═O |
| 1-2 | 3,5-Cl2 | O | H | — | CH2 | O | C═O |
| 1-3 | 3,5-Br2 | O | H | — | CH2 | O | C═O |
| 1-4 | 3,5-I2 | O | H | — | CH2 | O | C═O |
| 1-5 | 3-F | O | H | — | CH2 | O | C═O |
| 1-6 | 3-Cl | O | H | — | CH2 | O | C═O |
| 1-7 | 3-Br | O | H | — | CH2 | O | C═O |
| 1-8 | 3-CF3 | O | H | — | CH2 | O | C═O |
| 1-9 | 4-CF3 | O | H | — | CH2 | O | C═O |
| 1-10 | 3,5-(CF3)2 | O | H | — | CH2 | O | C═O |
| 1-11 | 3-NO2 | O | H | — | CH2 | O | C═O |
| 1-12 | 3-CH3 | O | H | — | CH2 | O | C═O |
| 1-13 | 3-CH3O | O | H | — | CH2 | O | C═O |
| 1-14 | 3-CN | O | H | — | CH2 | O | C═O |
| 1-15 | 3-CF3O | O | H | — | CH2 | O | C═O |
| 1-16 | 3-CH3S | O | H | — | CH2 | O | C═O |
| 1-17 | 3-CH3S(O) | O | H | — | CH2 | O | C═O |
| 1-18 | 3-CH3S(O)2 | O | H | — | CH2 | O | C═O |
| 1-19 | 3-CF3S | O | H | — | CH2 | O | C═O |
| 1-20 | 3-CF3S(O) | O | H | — | CH2 | O | C═O |
| 1-21 | 3-CF3S(O)2 | O | H | — | CH2 | O | C═O |
| 1-22 | 3-OH | O | H | — | CH2 | O | C═O |
| 1-23 | 3-SH | O | H | — | CH2 | O | C═O |
| 1-24 | 3-NH2 | O | H | — | CH2 | O | C═O |
| 1-25 | 3-NHCOCH3 | O | H | — | CH2 | O | C═O |
| 1-26 | 3-NHCOCF3 | O | H | — | CH2 | O | C═O |
| 1-27 | 3-NHCO2CH3 | O | H | — | CH2 | O | C═O |
| 1-28 | 3-NHCO2-CH2CCl3 | O | H | — | CH2 | O | C═O |
| 1-29 | 3-NHSO2CH3 | O | H | — | CH2 | O | C═O |
| 1-30 | 3-NHSO2CF3 | O | H | — | CH2 | O | C═O |
| 1-31 | 3-NHCH3 | O | H | — | CH2 | O | C═O |
| 1-32 | 3-N(CH3)2 | O | H | — | CH2 | O | C═O |
| 1-33 | 3-NHCOBu-t | O | H | — | CH2 | O | C═O |
| 1-34 | 3-Cl, 4-CF3 | O | H | — | CH2 | O | C═O |
| 1-35 | 3-Cl, 5-CF3 | O | H | — | CH2 | O | C═O |
| 1-36 | 3-Cl, 5-SCF3 | O | H | — | CH2 | O | C═O |
| 1-37 | 3,4,5-F3 | O | H | — | CH2 | O | C═O |
| 1-38 | 3,4,5-Cl3 | O | H | — | CH2 | O | C═O |
| 1-39 | 3,4,5-Br3 | O | H | — | CH2 | O | C═O |
| 1-40 | 3,5-Cl2, 4-Br | O | H | — | CH2 | O | C═O |
| 1-41 | 3,5-Br2, 4-Cl | O | H | — | CH2 | O | C═O |
| 1-42 | 3,5-Cl2, 4-NH2 | O | H | — | CH2 | O | C═O |
| 1-43 | 3,5-Br2, 4-NH2 | O | H | — | CH2 | O | C═O |
| 1-44 | 3,4-Cl2, 5-CF3 | O | H | — | CH2 | O | C═O |
| 1-45 | 3,5-Cl2, 4-CF3 | O | H | — | CH2 | O | C═O |
| 1-46 | 3,5-Cl2 | O | H | — | CH2 | S | C═O |
| 1-47 | 3,4,5-Cl3 | O | H | — | CH2 | S | C═O |
| 1-48 | 3,5-Cl2 | O | H | — | CH2 | O | C═O |
| 1-49 | 3,4,5-Cl3 | O | H | — | CH2 | O | C═O |
| 1-50 | 3,5-Cl2 | O | H | — | CH2 | NH | C═O |
| 1-51 | 3,4,5-Cl3 | O | H | — | CH2 | NH | C═O |
| 1-52 | 3,5-Cl2 | O | H | — | CH2 | NCH3 | C═O |
| 1-53 | 3,4,5-Cl3 | O | H | — | CH2 | NCH3 | C═O |
| 1-54 | 3,5-Cl2 | O | H | — | CH2 | NC2H5 | C═O |
| 1-55 | 3,4,5-Cl3 | O | H | — | CH2 | NC2H5 | C═O |
| 1-56 | 3,5-Cl2 | O | H | — | CH2 | NCH2CF3 | C═O |
| 1-57 | 3,4,5-Cl3 | O | H | — | CH2 | NCH2CF3 | C═O |
| 1-58 | 3,5-Cl2 | O | H | — | CH2 | NCH2-(pyridin-2-yl) | C═O |
| 1-59 | 3,4,5-Cl3 | O | H | — | CH2 | NCH2-(pyridin-2-yl) | C═O |
| 1-60 | 3,5-Cl2 | O | H | — | CH2 | NC3H7-n | C═O |
| 1-61 | 3,5-Cl2 | O | H | — | CH2 | NC3H7-iso | C═O |
| 1-62 | 3,5-Cl2 | O | H | — | CH2 | NC3H7-cyclo | C═O |
| 1-63 | 3,5-Cl2 | O | H | — | CH2 | NC4H9-n | C═O |
| 1-64 | 3,5-Cl2 | O | H | — | CH2 | NC4H9-iso | C═O |
| 1-65 | 3,5-Cl2 | O | H | — | CH2 | NC4H9-tert | C═O |
| 1-66 | 3,5-Cl2 | O | H | — | CH2 | NCH2—(C3H7-cyclo) | C═O |
| 1-67 | 3,5-Cl2 | O | H | — | CH2 | NCH2CH═CH2 | C═O |
| 1-68 | 3,5-Cl2 | O | H | — | CH2 | NCH2CCH | C═O |
| 1-69 | 3,5-Cl2 | O | H | — | CH2 | NC2H4OCH3 | C═O |
| 1-70 | 3,5-Cl2 | O | H | — | CH2 | NCH2CH—(OCH3)2 | C═O |
| 1-71 | 3,5-Cl2 | O | H | — | CH2 | NCH2-(pyridin-3-yl) | C═O |
| 1-72 | 3,5-Cl2 | O | H | — | CH2 | NCH2-(pyridin-4-yl) | C═O |
| 1-73 | 3,5-Cl2 | O | H | — | CH2 | NC2H4Cl | C═O |
| 1-74 | 3,5-Cl2 | O | H | — | CH2 | NCH2CO2CH3 | C═O |
| 1-75 | 3,5-Cl2 | O | H | — | CH2 | NCH2CONH2 | C═O |
| 1-76 | 3,5-Cl2 | O | H | — | CH2 | NCH2CONHCH3 | C═O |
| 1-77 | 3,5-Cl2 | O | H | — | CH2 | NCH2(1,3-dioxolane)-2 | C═O |
| 1-78 | 3,5-Cl2 | O | H | — | CH2 | NCH2(6-chloro-pyridin-2-yl) | C═O |
| 1-79 | 3,5-Cl2 | O | H | — | CH2 | NCH2Ph | C═O |
| 1-80 | 3,5-Cl2 | O | H | — | CH2 | NCH2-(pyrimidin-2-yl) | C═O |
| 1-81 | 3,5-Cl2 | O | H | — | CH2 | NPh | C═O |
| 1-82 | 3,5-Cl2 | O | H | — | CH2 | NPh-2-Cl | C═O |
| 1-83 | 3,5-Cl2 | O | H | — | CH2 | NPh-3-Cl | C═O |
| 1-84 | 3,5-Cl2 | O | H | — | CH2 | NPh-4-Cl | C═O |
| 1-85 | 3,5-Cl2 | O | H | — | CH2 | NPh-2-CF3 | C═O |
| 1-86 | 3,5-Cl2 | O | H | — | CH2 | NPh-3-CF3 | C═O |
| 1-87 | 3,5-Cl2 | O | H | — | CH2 | NPh-4-CF3 | C═O |
| 1-88 | 3,5-Cl2 | O | H | — | CH2 | NPh-2,4-F2 | C═O |
| 1-89 | 3,5-Cl2 | O | H | — | CH2 | N(pyrimidin-2-yl) | C═O |
| 1-90 | 3,5-Cl2 | O | H | — | CH2 | N-(6-chloro-pyrimidin-2-yl) | C═O |
| 1-91 | 3,5-Cl2 | O | H | — | CH2 | N(1H-pyrazol-3) | C═O |
| 1-92 | 3,5-Cl2 | O | H | — | CH2 | N(1H-1,2,4-triazol-3) | C═O |
| 1-93 | 3,5-Cl2 | O | H | — | CH2 | N(1,3-thiazol-2-yl) | C═O |
| 1-94 | 3,5-Cl2 | O | H | — | CH2 | NCOH | C═O |
| 1-95 | 3,5-Cl2 | O | H | — | CH2 | NCOCH3 | C═O |
| 1-96 | 3,5-Cl2 | O | H | — | CH2 | NCOC2H5 | C═O |
| 1-97 | 3,5-Cl2 | O | H | — | CH2 | NCOCF3 | C═O |
| 1-98 | 3,5-Cl2 | O | H | — | CH2 | NCOPh | C═O |
| 1-99 | 3,5-Cl2 | O | H | — | CH2 | NCO2CH3 | C═O |
| 1-100 | 3,5-Cl2 | O | H | — | CH2 | NCO2C2H5 | C═O |
| 1-101 | 3,5-Cl2 | O | H | — | CH2 | NCO2C4H9-tert | C═O |
| 1-102 | 3,5-Cl2 | O | H | — | CH2 | NCONH2 | C═O |
| 1-103 | 3,5-Cl2 | O | H | — | CH2 | NCONHPh | C═O |
| 1-104 | 3,5-Cl2 | O | H | — | CH2 | NNH2 | C═O |
| 1-105 | 3,5-Cl2 | O | H | — | CH2 | NNHCH3 | C═O |
| 1-106 | 3,5-Cl2 | O | H | — | CH2 | NN(CH3)2 | C═O |
| 1-107 | 3,5-Cl2 | O | H | — | CH2 | NNCOCH3 | C═O |
| 1-108 | 3,5-Cl2 | O | H | — | CH2 | NNCOCF3 | C═O |
| 1-109 | 3,5-Cl2 | O | H | — | CH2 | NN═CH(CH3)2 | C═O |
| 1-110 | 3,5-Cl2 | O | H | — | CH2 | NOH | C═O |
| 1-111 | 3,5-Cl2 | O | H | — | CH2 | NOCH3 | C═O |
| 1-112 | 3,5-Cl2 | O | H | — | CH2 | NOCH2Ph | C═O |
| 1-113 | 3,5-Cl2 | O | H | — | CH2 | NOCOCH3 | C═O |
| 1-114 | 3,5-Cl2 | O | H | — | CH2 | O | C═NCH3 |
| 1-115 | 3,5-Cl2 | O | H | — | CH2 | O | C═NCH2(py2-yl) |
| 1-116 | 3,5-Cl2 | O | H | C═O | O | CH2 | — |
| 1-117 | 3,5-Cl2 | O | H | C═O | O | C═O | — |
| 1-118 | 3,5-Cl2 | O | H | C═O | S | CH2 | — |
| 1-119 | 3,5-Cl2 | O | H | CH2 | CH2 | O | C═O |
| 1-120 | 3,5-Cl2 | O | H | — | C═O | NCH3 | C═O |
| 1-121 | 3,5-Cl2 | O | H | — | CH2 | C═O | NH |
| 1-122 | 3,5-Cl2 | O | H | — | CHSCH3 | C═O | NH |
| 1-123 | 3,5-Cl2 | O | H | — | CHCH3 | C═O | NH |
| 1-124 | 3,5-Cl2 | O | H | — | C═O | C═O | NH |
| 1-125 | 3,5-Cl2 | O | H | CH2 | NH | NH | C═O |
| 1-126 | 3,5-Cl2 | O | H | CH2 | NCH3 | NH | C═O |
| 1-127 | 3,5-Cl2 | O | H | CH2 | NCH3 | NCH3 | C═O |
| 1-128 | 3,5-Cl2 | O | H | CH2 | NCH3 | NCOCH3 | C═O |
| 1-129 | 3,5-Cl2 | O | H | CH | N | NH | C═O |
| 1-130 | 3,5-Cl2 | O | H | CH | O | NH | C═O |
| 1-131 | 3,5-Cl2 | O | H | CH2 | O | CH2 | — |
| 1-132 | 3,4,5-Cl3 | O | H | CH2 | O | CH2 | — |
| 1-133 | 3,5-Cl2 | O | H | CH2 | S | CH2 | — |
| 1-134 | 3,5-Cl2 | O | H | O | CH2 | O | — |
| 1-135 | 3,5-Cl2 | O | 7-Cl | O | CH2 | O | — |
| 1-136 | 3,5-Cl2 | O | H | — | CH2 | CH2 | O |
| 1-137 | 3,5-Cl2 | O | H | — | CH | CH | O |
| 1-138 | 3,5-Cl2 | O | H | O | CH | CH | — |
| 1-139 | 3,5-Cl2 | O | H | — | CH | CH | S |
| 1-140 | 3,5-Cl2 | O | H | S | CH | CH | — |
| 1-141 | 3,5-Cl2 | O | H | — | CH2 | CH2 | NH |
| 1-142 | 3,5-Cl2 | O | H | — | CH2 | CH2 | NCH3 |
| 1-143 | 3,5-Cl2 | O | H | — | CH2 | CH2 | NCH2Ph |
| 1-144 | 3,5-Cl2 | O | H | — | CH2 | CH2 | NPh |
| 1-145 | 3,5-Cl2 | O | H | — | CH2 | CH2 | NCOCH3 |
| 1-146 | 3,5-Cl2 | O | H | — | CH2 | CH2 | NCOCF3 |
| 1-147 | 3,5-Cl2 | O | H | — | CH2 | CH2 | NCO2CH3 |
| 1-148 | 3,5-Cl2 | O | H | — | CH2 | NH | CH2 |
| 1-149 | 3,5-Cl2 | O | H | — | CH2 | NCH2Ph | CH2 |
| 1-150 | 3,5-Cl2 | O | H | — | CH2 | NCH2(py-2-yl) | CH2 |
| 1-151 | 3,5-Cl2 | O | H | — | CH2 | NCOCH3 | CH2 |
| 1-152 | 3,5-Cl2 | O | H | NH | CH2 | CH2 | — |
| 1-153 | 3,5-Cl2 | O | H | NCH3 | CH2 | CH2 | — |
| 1-154 | 3,5-Cl2 | O | H | NCO2CH3 | CH2 | CH2 | — |
| 1-155 | 3,5-Cl2 | O | H | — | CH | CH | NH |
| 1-156 | 3,5-Cl2 | O | H | — | CH | CH | NCH3 |
| 1-157 | 3,5-Cl2 | O | H | — | CH | CH | NCO2C4H9-tert |
| 1-158 | 3,5-Cl2 | O | H | — | CH | N | NH |
| 1-159 | 3,5-Cl2 | O | H | — | CH | N | NCH3 |
| 1-160 | 3,5-Cl2 | O | H | — | CH | N | NPh |
| 1-161 | 3,5-Cl2 | O | H | — | CH | N | NCO2—C4H9-tert |
| 1-162 | 3,5-Cl2 | O | H | NH | N | CH | — |
| 1-163 | 3,5-Cl2 | O | H | N | CH | CH | CH |
| 1-164 | 3,5-Cl2 | O | H | CH | CH | CH | N |
| 1-165 | 3,5-Cl2 | O | H | NH | CH2 | CH2 | CH2 |
| 1-166 | 3,5-Cl2 | O | H | NCH3 | CH2 | CH2 | CH2 |
| 1-167 | 3,5-Cl2 | O | H | NCOCH3 | CH2 | CH2 | CH2 |
| 1-168 | 3,5-Cl2 | O | H | NCOCF3 | CH2 | CH2 | CH2 |
| 1-169 | 3,5-Cl2 | O | H | CH2 | CH2 | CH2 | NH |
| 1-170 | 3,5-Cl2 | O | H | CH2 | CH2 | CH2 | NCH3 |
| 1-171 | 3,5-Cl2 | O | H | CH2 | CH2 | CH2 | NCOCH3 |
| 1-172 | 3,5-Cl2 | O | H | CH2 | CH2 | CH2 | NCOCF3 |
| 1-173 | 3,5-Cl2 | O | H | CH2 | CH2 | CH2 | NCO2CH3 |
| 1-174 | 3,5-Cl2 | O | H | CH2 | CH2 | CH2 | NCONH2 |
| 1-175 | 3,5-Cl2 | O | H | N | CH | CH | N |
| 1-176 | 3,5-Cl2 | O | H | NH | CH2 | CH2 | NH |
| 1-177 | 3,5-Cl2 | O | H | NCH3 | CH2 | CH2 | NCH3 |
| 1-178 | 3,5-Cl2 | O | H | NCOCH3 | CH | CH | NCOCH3 |
| 1-179 | 3,5-Cl2 | O | H | — | N | CH | NH |
| 1-180 | 3,5-Cl2 | O | H | — | N | N | NH |
| 1-181 | 3,5-Cl2 | O | H | — | NH | CH2 | O |
| 1-182 | 3,5-Cl2 | O | H | — | N | CH | O |
| 1-183 | 3,5-Cl2 | O | H | — | N | CCH3 | O |
| 1-184 | 3,5-Cl2 | O | H | — | NH | C═O | O |
| 1-185 | 3,5-Cl2 | O | H | — | NH | C═S | O |
| 1-186 | 3,5-Cl2 | O | H | — | NH | S═O | O |
| 1-187 | 3,5-Cl2 | O | H | — | NH | S(═O)2 | O |
| 1-188 | 3,5-Cl2 | O | H | — | O | CH2 | NH |
| 1-189 | 3,5-Cl2 | O | H | — | O | CH | N |
| 1-190 | 3,5-Cl2 | O | H | — | O | CCH3 | N |
| 1-191 | 3,5-Cl2 | O | H | — | O | C═O | NH |
| 1-192 | 3,5-Cl2 | O | H | — | O | C═S | NH |
| 1-193 | 3,5-Cl2 | O | H | — | O | S═O | NH |
| 1-194 | 3,5-Cl2 | O | H | — | O | S(═O)2 | NH |
| 1-195 | 3,5-Cl2 | O | H | CH2 | O | CH2 | NH |
| 1-196 | 3,5-Cl2 | O | H | CH2 | O | CH | N |
| 1-197 | 3,5-Cl2 | O | H | CH2 | O | CCH3 | N |
| 1-198 | 3,5-Cl2 | O | H | CH2 | O | C═O | NH |
| 1-199 | 3,5-Cl2 | O | H | CH2 | O | C═S | NH |
| 1-200 | 3,5-Cl2 | O | H | CH2 | O | S═O | NH |
| 1-201 | 3,5-Cl2 | O | H | CH2 | O | S(═O)2 | NH |
| 1-202 | 3,5-Cl2 | O | H | — | N | CH | S |
| 1-203 | 3,5-Cl2 | O | H | — | N | CCH3 | S |
| 1-204 | 3,5-Cl2 | O | H | — | NH | C═O | S |
| 1-205 | 3,5-Cl2 | O | H | — | NCH3 | C═O | S |
| 1-206 | 3,5-Cl2 | O | H | — | NH | C═O | NH |
| 1-207 | 3,5-Cl2 | O | H | — | NCH3 | C═O | NCH3 |
| 1-208 | 3,5-Cl2 | O | H | — | N | CH | NH |
| 1-209 | 3,5-Cl2 | O | H | — | CH2 | CH2 | C═O |
| 1-210 | 3,4,5-Cl3 | O | H | — | CH2 | CH2 | C═O |
| 1-211 | 3,5-Cl2 | O | H | — | CH2 | CH2 | C═NOH |
| 1-212 | 3,4,5-Cl3 | O | H | — | CH2 | CH2 | C═NOH |
| 1-213 | 3,5-Cl2 | O | H | — | CH2 | CH2 | C═NOCH3 |
| 1-214 | 3,4,5-Cl3 | O | H | — | CH2 | CH2 | C═NOCH3 |
| 1-215 | 3,5-Cl2 | O | H | — | CH2 | CH2 | C═NNH2 |
| 1-216 | 3,4,5-Cl3 | O | H | — | CH2 | CH2 | C═NNH2 |
| 1-217 | 3,5-Cl2 | O | H | — | CH2 | CH2 | C═NNH—CH3 |
| 1-218 | 3,4,5-Cl3 | O | H | — | CH2 | CH2 | C═NNH—CH3 |
| 1-219 | 3,5-Cl2 | O | H | — | CH2 | CH2 | C═NN—(CH3)2 |
| 1-220 | 3,4,5-Cl3 | O | H | — | CH2 | CH2 | C═NN—(CH3)2 |
| 1-221 | 3,5-Cl2 | O | H | — | CH2 | N—COCH3 | C═O |
| 1-222 | 3,5-Cl2 | O | H | CH2 | CH2 | CH2 | C═O |
| 1-223 | 3,5-Cl2 | O | H | CH2 | CH2 | CH2 | C═NOH |
| 1-224 | 3,5-Cl2 | O | H | CH2 | CH2 | CH2 | C═NOCH3 |
| 1-225 | 3,5-Cl2 | O | H | CH2 | CH2 | CH2 | C═NNH2 |
| 1-226 | 3,5-Cl2 | O | H | CH2 | CH2 | CH2 | C═NNH—CH3 |
| 1-227 | 3,5-Cl2 | O | H | CH2 | CH2 | CH2 | C═NN—(CH3)2 |
| 1-228 | 3,5-Cl2 | CH2 | H | — | CH2 | O | C═O |
| 1-229 | 3,4,5-Cl3 | CH2 | H | — | CH2 | O | C═O |
| 1-230 | 3,5-Cl2 | NCH3 | H | — | CH2 | O | C═O |
| 1-231 | 3,4,5-Cl3 | NCH3 | H | — | CH2 | O | C═O |
| 1-232 | 3,5-Cl2 | O | H | — | CH2 | O | C═NH |
| 1-233 | 3,4,5-Cl3 | NCH3 | H | — | CH2 | O | C═NH |
| 1-234 | 3,5-Cl2 | O | H | — | CH2 | O | C═NHCO—CH3 |
| 1-235 | 3,5-Cl2 | O | H | — | CH2 | NCH2-(py-2-yl) | C═NHCO—CH3 |
| 1-236 | 3,5-Cl2 | O | H | — | CH2 | O | C═CH2 |
| 1-237 | 3,4,5-Cl3 | O | H | — | CH2 | O | C═CH2 |
| 1-238 | 3,5-Cl2 | O | H | — | CH2 | O | CH—OH |
| 1-239 | 3,4,5-Cl3 | O | H | — | CH2 | O | CH—OH |
| 1-240 | 3,5-Cl2 | O | H | — | CH2 | CH2 | NCH2(py-2-yl) |
| 1-241 | 3,5-Cl2 | O | H | — | CH2 | CH2 | C═NOCH2(py-2-yl |
| 1-242 | 3,5-Cl2 | O | H | — | CH2 | CH2 | C═NNHCOCH3 |
| 1-243 | 3,5-Cl2 | O | H | — | CH2 | CH2 | C═NNHCH2CF3 |
| 1-244 | 3,5-Cl2 | O | H | — | CH2 | CH2 | C═NOCH2CF3 |
| 1-245 | 3,5-Cl2 | O | H | — | CH2 | O | C═NH |
| 1-246 | 3,5-Cl2 | O | H | — | CH2 | CHCl | C═O |
| 1-247 | 3,5-Cl2 | O | H | — | CCl | CCl | NH |
| 1-248 | 3,5-Cl2 | O | H | — | NH | C═O | CH2 |
| 1-249 | 3,5-Cl2 | O | H | — | C═O | NN═CH(CH3)2 | CH2 |
| 1-250 | 3,5-Cl2 | O | H | CH2 | CH2 | NH | C═O |
| 1-251 | 3,5-Cl2 | O | H | CH2 | CH2 | C═O | NH |
| 1-252 | 3,5-Cl2 | O | H | CH2 | CH2 | CH2 | C═NOCH2—CF3 |
| 1-253 | 3,5-Cl2 | O | H | O | CH2 | CH2 | O |
| 1-254 | 3,4,5-Cl3 | O | H | N | CH2 | CH2 | CH2 |
| 1-255 | 3,5-Cl2 | O | H | CH | CH | CCN | N |
| 1-256 | 3,5-Cl2 | O | H | CCl | CH | CH | N |
| 1-257 | 3,5-Cl2 | O | H | CH | CH | CH | N—O |
| 1-258 | 3,5-Cl2 | O | H | CH | CH | N | NCH2-(py-2-yl) |
| 1-259 | 3,5-Cl2 | O | H | CH | N | NCOCH3 | C═O |
| 1-260 | 3,5-Cl2 | O | H | CH | N | NCH2(py-2-yl) | C═O |
| 1-261 | 3,5-Cl2 | O | H | — | C═O | NCH2(py-2-yl) | CH2 |
| 1-262 | 3,4,5-Cl3 | O | H | — | CH2 | NCH2(py-2-yl) | CH2 |
| 1-263 | 3,5-Cl2 | O | H | — | C═NCOCH3 | NCH3 | CH2 |
| 1-264 | 3,5-Cl2 | O | H | — | CH2 | NCH2(py-2-yl) | C═NCO—CH3 |
| 1-265 | 3,5-Cl2 | O | H | — | C═O | NCH3 | CH2 |
| 1-266 | 3,5-Cl2 | O | H | — | C═NCH3 | O | CH2 |
| 1-267 | 3,5-Cl2 | O | H | — | C═NH | NCH2(py-2-yl) | CH2 |
| 1-268 | 3,4,5-Cl3 | O | H | — | CH2 | NCH2CF3 | C═O |
| 1-269 | 3,5-Cl2 | O | H | — | CH2 | N(Ph-4-CF3) | C═O |
| 1-270 | 3,5-Cl2 | O | H | — | CH2 | N(Ph-3-CF3) | C═O |
| 1-271 | 3,5-Cl2 | O | H | — | CH2 | NCH2CH2F | C═O |
| 1-272 | 3,5-Cl2 | O | H | — | CH2 | NCH2CF2CF3 | C═O |
| 1-273 | 3,4,5-Cl3 | O | H | CH | N | NH | C═O |
| 1-274 | 3,4,5-Cl3 | O | H | CH | N | NCOCH3 | C═O |
| 1-275 | 3,4,5-Cl3 | O | H | CH | N | NCOC2H5 | C═O |
| 1-276 | 3,4,5-Cl3 | O | H | CH | N | NCOCH2CF3 | C═O |
| 1-277 | 3,4,5-Cl3 | O | H | CH | N | NCOPr-c | C═O |
| 1-278 | 3,4,5-Cl3 | O | H | CH | N | NCOCF3 | C═O |
| 1-279 | 3,4,5-Cl3 | O | H | CH2 | NH | NH | C═O |
| 1-280 | 3,4,5-Cl3 | O | H | CH2 | NCH3 | NH | C═O |
| 1-281 | 3,4,5-Cl3 | O | H | CH2 | NCH3 | NCH3 | C═O |
| 1-282 | 3,4,5-Cl3 | O | H | CH2 | NCH3 | NCOCH3 | C═O |
| 1-283 | 3,5-(CF3)2 | O | H | CH | N | NH | C═O |
| 1-284 | 3,5-(CF3)2 | O | H | CH | N | NCOCH3 | C═O |
| 1-285 | 3,5-Br2 | O | H | CH2 | NH | NH | C═O |
| 1-286 | 3-Cl, 5-CF3 | O | H | CH2 | NCH3 | NH | C═O |
| 1-287 | 3-CF3 | O | H | CH2 | NCH3 | NCH3 | C═O |
| 1-288 | 3,5-Cl2 | O | H | CH2 | O | NH | C═O |
| 1-289 | 3,4,5-Cl3 | O | H | CH2 | O | NCOCH3 | C═O |
| 1-290 | 3,5-Cl2 | O | H | CH2 | NCO2-C4H9-t | O | C═O |
| 1-291 | 3,4-Cl2, 5-CF3 | O | H | CH2 | N | O | C═O |
| 1-292 | 3,4,5-Cl3 | O | H | CH2 | N | O | C═O |
| 1-293 | 3,4,5-Cl3 | O | H | CH | CH | CBr | N |
| 1-294 | 3,4,5-Cl3 | O | H | CH | CH | CCN | N |
| 1-295 | 3,5-Cl2 | O | H | CH | CH | CCl | N |

TABLE 2

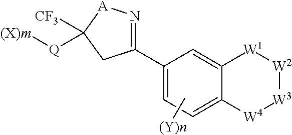

| Example | (X)m-Q | A | (Y)n | W¹ | W² | W³ | W⁴ |
|---|---|---|---|---|---|---|---|
| 2-1 | Q-2 | O | H | — | CH2 | O | C═O |
| 2-2 | Q-3 | O | H | — | CH2 | O | C═O |
| 2-3 | Q-4 | O | H | — | CH2 | O | C═O |
| 2-4 | Q-5 | O | H | — | CH2 | O | C═O |
| 2-5 | Q-6 | O | H | — | CH2 | O | C═O |
| 2-6 | Q-7 | O | H | — | CH2 | O | C═O |
| 2-7 | Q-8 | O | H | — | CH2 | O | C═O |
| 2-8 | Q-9 | O | H | — | CH2 | O | C═O |
| 2-9 | Q-10 | O | H | — | CH2 | O | C═O |
| 2-10 | Q-11 | O | H | — | CH2 | O | C═O |
| 2-11 | Q-12 | O | H | — | CH2 | O | C═O |
| 2-12 | Q-13 | O | H | — | CH2 | O | C═O |
| 2-13 | Q-14 | O | H | — | CH2 | O | C═O |
| 2-14 | Q-15 | O | H | — | CH2 | O | C═O |
| 2-15 | Q-16 | O | H | — | CH2 | O | C═O |
| 2-16 | Q-17 | O | H | — | CH2 | O | C═O |
| 2-17 | Q-18 | O | H | — | CH2 | O | C═O |
| 2-18 | Q-19 | O | H | — | CH2 | O | C═O |
| 2-19 | Q-20 | O | H | — | CH2 | O | C═O |
| 2-20 | Q-21 | O | H | — | CH2 | O | C═O |
| 2-21 | Q-22 | O | H | — | CH2 | O | C═O |
| 2-22 | Q-23 | O | H | — | CH2 | O | C═O |
| 2-23 | Q-24 | O | H | — | CH2 | O | C═O |
| 2-24 | Q-25 | O | H | — | CH2 | O | C═O |
| 2-25 | Q-26 | O | H | — | CH2 | O | C═O |
| 2-26 | Q-27 | O | H | — | CH2 | O | C═O |
| 2-27 | Q-28 | O | H | — | CH2 | O | C═O |
| 2-28 | Q-29 | O | H | — | CH2 | O | C═O |
| 2-29 | Q-30 | O | H | — | CH2 | O | C═O |
| 2-30 | Q-31 | O | H | — | CH2 | O | C═O |
| 2-31 | Q-32 | O | H | — | CH2 | O | C═O |
| 2-32 | Q-33 | O | H | — | CH2 | O | C═O |
| 2-33 | Q-34 | O | H | — | CH2 | O | C═O |
| 2-34 | Q-35 | O | H | — | CH2 | O | C═O |
| 2-35 | Q-36 | O | H | — | CH2 | O | C═O |
| 2-36 | Q-37 | O | H | — | CH2 | O | C═O |
| 2-37 | Q-38 | O | H | — | CH2 | O | C═O |
| 2-38 | Q-39 | O | H | — | CH2 | O | C═O |
| 2-39 | Q-40 | O | H | — | CH2 | O | C═O |
| 2-40 | Q-41 | O | H | — | CH2 | O | C═O |
| 2-41 | Q-42 | O | H | — | CH2 | O | C═O |
| 2-42 | 2-Cl-(Q-42)-4-yl | O | H | — | CH2 | O | C═O |
| 2-43 | 2,6-2Cl-(Q-42)-4-yl | O | H | — | CH2 | O | C═O |
| 2-44 | 2-CF3-(Q-42)-4-yl | O | H | — | CH2 | O | C═O |
| 2-45 | 2-C2F5-(Q-42)-4-yl | O | H | — | CH2 | O | C═O |
| 2-46 | Q-44 | O | H | — | CH2 | O | C═O |
| 2-47 | 2-Cl-(Q-44)-5-yl | O | H | — | CH2 | O | C═O |
| 2-48 | Q-43 | O | H | — | CH2 | O | C═O |
| 2-49 | 2-Cl-(Q-43)-6-yl | O | H | — | CH2 | O | C═O |
| 2-50 | Q-45 | O | H | — | CH2 | O | C═O |
| 2-51 | 3-Cl-(Q-45)-6-yl | O | H | — | CH2 | O | C═O |
| 2-52 | Q-47 | O | H | — | CH2 | O | C═O |
| 2-53 | 2-Cl-(Q-47)-5-yl | O | H | — | CH2 | O | C═O |
| 2-54 | Q-46 | O | H | — | CH2 | O | C═O |
| 2-55 | 4,6-2Cl-(Q-46)-2-yl | O | H | — | CH2 | O | C═O |
| 2-56 | Q-48 | O | H | — | CH2 | O | C═O |
| 2-57 | Q-49 | O | H | — | CH2 | O | C═O |
| 2-58 | Q-50 | O | H | — | CH2 | O | C═O |
| 2-59 | Q-51 | O | H | — | CH2 | O | C═O |
| 2-60 | Q-52 | O | H | — | CH2 | O | C═O |
| 2-61 | Q-53 | O | H | — | CH2 | O | C═O |
| 2-62 | Q-54 | O | H | — | CH2 | O | C═O |
| 2-63 | Q-2 | O | H | — | CH2 | NCH2(py-2-yl) | C═O |
| 2-64 | Q-3 | O | H | — | CH2 | NCH2(py-2-yl) | C═O |
| 2-65 | Q-4 | O | H | — | CH2 | NCH2(py-2-yl) | C═O |
| 2-66 | Q-5 | O | H | — | CH2 | NCH2(py-2-yl) | C═O |
| 2-67 | Q-6 | O | H | — | CH2 | NCH2(py-2-yl) | C═O |
| 2-68 | Q-7 | O | H | — | CH2 | NCH2(py-2-yl) | C═O |
| 2-69 | Q-8 | O | H | — | CH2 | NCH2(py-2-yl) | C═O |
| 2-70 | Q-9 | O | H | — | CH2 | NCH2(py-2-yl) | C═O |
| 2-71 | Q-10 | O | H | — | CH2 | NCH2(py-2-yl) | C═O |
| 2-72 | Q-11 | O | H | — | CH2 | NCH2(py-2-yl) | C═O |
| 2-73 | Q-12 | O | H | — | CH2 | NCH2(py-2-yl) | C═O |
| 2-74 | Q-13 | O | H | — | CH2 | NCH2(py-2-yl) | C═O |
| 2-75 | Q-14 | O | H | — | CH2 | NCH2(py-2-yl) | C═O |
| 2-76 | Q-15 | O | H | — | CH2 | NCH2(py-2-yl) | C═O |
| 2-77 | Q-16 | O | H | — | CH2 | NCH2(py-2-yl) | C═O |
| 2-78 | Q-17 | O | H | — | CH2 | NCH2(py-2-yl) | C═O |
| 2-79 | Q-18 | O | H | — | CH2 | NCH2(py-2-yl) | C═O |
| 2-80 | Q-19 | O | H | — | CH2 | NCH2(py-2-yl) | C═O |
| 2-81 | Q-20 | O | H | — | CH2 | NCH2(py-2-yl) | C═O |
| 2-82 | Q-21 | O | H | — | CH2 | NCH2(py-2-yl) | C═O |
| 2-83 | Q-22 | O | H | — | CH2 | NCH2(py-2-yl) | C═O |
| 2-84 | Q-23 | O | H | — | CH2 | NCH2(py-2-yl) | C═O |
| 2-85 | Q-24 | O | H | — | CH2 | NCH2(py-2-yl) | C═O |
| 2-86 | Q-25 | O | H | — | CH2 | NCH2(py-2-yl) | C═O |
| 2-87 | Q-26 | O | H | — | CH2 | NCH2(py-2-yl) | C═O |
| 2-88 | Q-27 | O | H | — | CH2 | NCH2(py-2-yl) | C═O |
| 2-89 | Q-28 | O | H | — | CH2 | NCH2(py-2-yl) | C═O |
| 2-90 | Q-29 | O | H | — | CH2 | NCH2(py-2-yl) | C═O |
| 2-91 | Q-30 | O | H | — | CH2 | NCH2(py-2-yl) | C═O |
| 2-92 | Q-31 | O | H | — | CH2 | NCH2(py-2-yl) | C═O |
| 2-93 | Q-32 | O | H | — | CH2 | NCH2(py-2-yl) | C═O |
| 2-94 | Q-33 | O | H | — | CH2 | NCH2(py-2-yl) | C═O |
| 2-95 | Q-34 | O | H | — | CH2 | NCH2(py-2-yl) | C═O |
| 2-96 | Q-35 | O | H | — | CH2 | NCH2(py-2-yl) | C═O |
| 2-97 | Q-36 | O | H | — | CH2 | NCH2(py-2-yl) | C═O |
| 2-98 | Q-37 | O | H | — | CH2 | NCH2(py-2-yl) | C═O |
| 2-99 | Q-38 | O | H | — | CH2 | NCH2(py-2-yl) | C═O |
| 2-100 | Q-39 | O | H | — | CH2 | NCH2(py-2-yl) | C═O |
| 2-101 | Q-40 | O | H | — | CH2 | NCH2(py-2-yl) | C═O |
| 2-102 | Q-41 | O | H | — | CH2 | NCH2(py-2-yl) | C═O |
| 2-103 | Q-42 | O | H | — | CH2 | NCH2(py-2-yl) | C═O |
| 2-104 | 2-Cl-(Q-42)-4-yl | O | H | — | CH2 | NCH2(py-2-yl) | C═O |
| 2-105 | 2,6-2Cl-(Q-42)-4-yl | O | H | — | CH2 | NCH2(py-2-yl) | C═O |
| 2-106 | 2-CF3-(Q-42)-4-yl | O | H | — | CH2 | NCH2(py-2-yl) | C═O |
| 2-107 | 2-C2F5-(Q-42)-4-yl | O | H | — | CH2 | NCH2(py-2-yl) | C═O |
| 2-108 | Q-44 | O | H | — | CH2 | NCH2(py-2-yl) | C═O |
| 2-109 | 2-Cl-(Q-44)-5-yl | O | H | — | CH2 | NCH2(py-2-yl) | C═O |
| 2-110 | Q-43 | O | H | — | CH2 | NCH2(py-2-yl) | C═O |
| 2-111 | 2-Cl-(Q-43)-6-yl | O | H | — | CH2 | NCH2(py-2-yl) | C═O |
| 2-112 | Q-45 | O | H | — | CH2 | NCH2(py-2-yl) | C═O |
| 2-113 | 3-Cl-(Q-45)-6-yl | O | H | — | CH2 | NCH2(py-2-yl) | C═O |
| 2-114 | Q-47 | O | H | — | CH2 | NCH2(py-2-yl) | C═O |
| 2-115 | 2-Cl-(Q-47)-5-yl | O | H | — | CH2 | NCH2(py-2-yl) | C═O |
| 2-116 | Q-46 | O | H | — | CH2 | NCH2(py-2-yl) | C═O |
| 2-117 | 4,6-2Cl-(Q-46)-2-yl | O | H | — | CH2 | NCH2(py-2-yl) | C═O |
| 2-118 | Q-48 | O | H | — | CH2 | NCH2(py-2-yl) | C═O |
| 2-119 | Q-49 | O | H | — | CH2 | NCH2(py-2-yl) | C═O |
| 2-120 | Q-50 | O | H | — | CH2 | NCH2(py-2-yl) | C═O |
| 2-121 | Q-51 | O | H | — | CH2 | NCH2(py-2-yl) | C═O |
| 2-122 | Q-52 | O | H | — | CH2 | NCH2(py-2-yl) | C═O |
| 2-123 | Q-53 | O | H | — | CH2 | NCH2(py-2-yl) | C═O |
| 2-124 | Q-54 | O | H | — | CH2 | NCH2(py-2-yl) | C═O |
| 2-125 | 2,6-2Cl-(Q-42)-4-yl | O | H | — | CH2 | CH2 | CHNH2 |
| 2-126 | 2,6-2Cl-(Q-42)-4-yl | O | H | — | CH2 | CH2 | CHNH(C═O)CH3 |
| 2-127 | 2,6-2Cl-(Q-42)-4-yl | O | H | — | CH2 | CH2 | CHNH(C═O)C2H5 |
| 2-128 | 2,6-2Cl-(Q-42)-4-yl | O | H | — | CH2 | CH2 | CHNH(C═O)CH2CF3 |
| 2-129 | 2,6-2Cl-(Q-42)-4-yl | O | H | — | CH2 | CH2 | CHNH(C═O)Pr-c |
| 2-130 | 2,6-2Cl-(Q-42)-4-yl | O | H | — | CH2 | CH2 | CHNH(C═O)Pr-i |
| 2-131 | 2,6-2Cl-(Q-42)-4-yl | O | H | — | CH2 | CH2 | CHNH(C═O)CH2Pr-c |
| 2-132 | 2,6-2Cl-(Q-42)-4-yl | O | H | — | CH2 | CH2 | CHNH(C═O)CH2SMe |
| 2-133 | 2,6-2Cl-(Q-42)-4-yl | O | H | — | CH2 | CH2 | CHNH(C═O)CH2S(═O)Me |
| 2-134 | 2,6-2Cl-(Q-42)-4-yl | O | H | — | CH2 | CH2 | CHNH(C═O)CH2S(═O)2Me |
| 2-135 | 2,6-2Cl-(Q-42)-4-yl | O | H | — | CH2 | CH2 | CHNH(C═O)CF3 |
| 2-136 | 2,6-2Cl-(Q-42)-4-yl | O | H | — | CH2 | CH2 | CHNH(C═O)NHCH3 |
| 2-137 | 2,6-2Cl-(Q-42)-4-yl | O | H | — | CH2 | CH2 | CHNH(C═O)NHC2H5 |
| 2-138 | 2,6-2Cl-(Q-42)-4-yl | O | H | — | CH2 | CH2 | CHNH(C═O)NHCH2CCH |
| 2-139 | 2,6-2Cl-(Q-42)-4-yl | O | H | — | CH2 | CH2 | CHNH(C═S)C2H5 |
| 2-140 | 2,6-2Cl-(Q-42)-4-yl | O | H | — | CH2 | CH2 | CHNH(C═S)NHCH3 |
| 2-141 | 2,6-2Cl-(Q-42)-4-yl | O | H | — | CH2 | CH2 | CHNH(C═O)OCH3 |
| 2-142 | 2,6-2Cl-(Q-42)-4-yl | O | H | — | CH2 | CH2 | CHNH(C═O)OC4H9-tert |
| 2-143 | 2,6-2Cl-(Q-42)-4-yl | CH2 | H | — | CH2 | CH2 | CHNH2 |
| 2-144 | 2,6-2Cl-(Q-42)-4-yl | CH2 | H | — | CH2 | CH2 | CHNH(C═O)CH3 |
| 2-145 | 2,6-2Cl-(Q-42)-4-yl | CH2 | H | — | CH2 | CH2 | CHNH(C═O)C2H5 |
| 2-146 | 2,6-2Cl-(Q-42)-4-yl | CH2 | H | — | CH2 | CH2 | CHNH(C═O)CH2CF3 |
| 2-147 | 2,6-2Cl-(Q-42)-4-yl | CH2 | H | — | CH2 | CH2 | CHNH(C═O)Pr-c |
| 2-148 | 2,6-2Cl-(Q-42)-4-yl | CH2 | H | — | CH2 | CH2 | CHNH(C═O)Pr-i |
| 2-149 | 2,6-2Cl-(Q-42)-4-yl | CH2 | H | — | CH2 | CH2 | CHNH(C═O)CH2Pr-c |
| 2-150 | 2,6-2Cl-(Q-42)-4-yl | CH2 | H | — | CH2 | CH2 | CHNH(C═O)CH2SMe |
| 2-151 | 2,6-2Cl-(Q-42)-4-yl | CH2 | H | — | CH2 | CH2 | CHNH(C═O)CH2S(═O)Me |
| 2-152 | 2,6-2Cl-(Q-42)-4-yl | CH2 | H | — | CH2 | CH2 | CHNH(C═O)CH2S(═O)2Me |
| 2-153 | 2,6-2Cl-(Q-42)-4-yl | CH2 | H | — | CH2 | CH2 | CHNH(C═O)CF3 |
| 2-154 | 2,6-2Cl-(Q-42)-4-yl | CH2 | H | — | CH2 | CH2 | CHNH(C═O)NHCH3 |
| 2-155 | 2,6-2Cl-(Q-42)-4-yl | CH2 | H | — | CH2 | CH2 | CHNH(C═O)NHC2H5 |
| 2-156 | 2,6-2Cl-(Q-42)-4-yl | CH2 | H | — | CH2 | CH2 | CHNH(C═O)NHCH2CCH |
| 2-157 | 2,6-2Cl-(Q-42)-4-yl | CH2 | H | — | CH2 | CH2 | CHNH(C═S)C2H5 |
| 2-158 | 2,6-2Cl-(Q-42)-4-yl | CH2 | H | — | CH2 | CH2 | CHNH(C═S)NHCH3 |
| 2-159 | 2,6-2Cl-(Q-42)-4-yl | CH2 | H | — | CH2 | CH2 | CHNH(C═O)OCH3 |
| 2-160 | 2,6-2Cl-(Q-42)-4-yl | CH2 | H | — | CH2 | CH2 | CHNH(C═O)OC4H9-tert |
| 2-161 | 2,6-2Cl-(Q-42)-4-yl | O | H | CH2 | CH2 | CH2 | CHNH2 |
| 2-162 | 2,6-2Cl-(Q-42)-4-yl | O | H | CH2 | CH2 | CH2 | CHNH(C═O)CH3 |
| 2-163 | 2,6-2Cl-(Q-42)-4-yl | O | H | CH2 | CH2 | CH2 | CHNH(C═O)C2H5 |
| 2-164 | 2,6-2Cl-(Q-42)-4-yl | O | H | CH2 | CH2 | CH2 | CHNH(C═O)CH2CF3 |
| 2-165 | 2,6-2Cl-(Q-42)-4-yl | O | H | CH2 | CH2 | CH2 | CHNH(C═O)Pr-c |
| 2-166 | 2,6-2Cl-(Q-42)-4-yl | O | H | CH2 | CH2 | CH2 | CHNH(C═O)CH2S(═O)2Me |
| 2-167 | 2,6-2Cl-(Q-42)-4-yl | O | H | CH2 | CH2 | CH2 | CHNH(C═O)NHC2H5 |
| 2-168 | 2,6-2Cl-(Q-42)-4-yl | O | H | CH2 | CH2 | CH2 | CHNH(C═O)OC4H9-tert |
| 2-169 | 2-CF3-(Q-42)-4-yl | O | H | CH2 | CH2 | CH2 | CHNH2 |
| 2-170 | 2-CF3-(Q-42)-4-yl | O | H | CH2 | CH2 | CH2 | CHNH(C═O)CH3 |
| 2-171 | 2-CF3-(Q-42)-4-yl | O | H | CH2 | CH2 | CH2 | CHNH(C═O)C2H5 |
| 2-172 | 2-CF3-(Q-42)-4-yl | O | H | CH2 | CH2 | CH2 | CHNH(C═O)CH2CF3 |
| 2-173 | 2-CF3-(Q-42)-4-yl | O | H | CH2 | CH2 | CH2 | CHNH(C═O)Pr-c |
| 2-174 | 2-CF3-(Q-42)-4-yl | O | H | CH2 | CH2 | CH2 | CHNH(C═O)CH2S(═O)2Me |
| 2-175 | 2-CF3-(Q-42)-4-yl | O | H | CH2 | CH2 | CH2 | CHNH(C═O)NHC2H5 |
| 2-176 | 2-CF3-(Q-42)-4-yl | O | H | CH2 | CH2 | CH2 | CHNH(C═O)OC4H9-tert |
| 2-177 | 2-C2F5-(Q-42)-4-yl | O | H | CH2 | CH2 | CH2 | CHNH2 |
| 2-178 | 2-C2F5-(Q-42)-4-yl | O | H | CH2 | CH2 | CH2 | CHNH(C═O)CH3 |
| 2-179 | 2-C2F5-(Q-42)-4-yl | O | H | CH2 | CH2 | CH2 | CHNH(C═O)C2H5 |
| 2-180 | 2-C2F5-(Q-42)-4-yl | O | H | CH2 | CH2 | CH2 | CHNH(C═O)CH2CF3 |
| 2-181 | 2-C2F5-(Q-42)-4-yl | O | H | CH2 | CH2 | CH2 | CHNH(C═O)Pr-c |
| 2-182 | 2-C2F5-(Q-42)-4-yl | O | H | CH2 | CH2 | CH2 | CHNH(C═O)CH2S(═O)2Me |
| 2-183 | 2-C2F5-(Q-42)-4-yl | O | H | CH2 | CH2 | CH2 | CHNH(C═O)NHC2H5 |
| 2-184 | 2-C2F5-(Q-42)-4-yl | O | H | CH2 | CH2 | CH2 | CHNH(C═O)OC4H9-tert |

TABLE 3

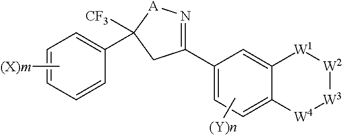

| Example | (X)m | A | (Y)n | W¹ | W² | W³ | W⁴ |
|---|---|---|---|---|---|---|---|
| 3-1 | 3,5-Cl2 | O | H | — | CH2 | CH2 | CHNH2 |
| 3-2 | 3,5-Cl2 | O | H | — | CH2 | CH2 | CHNH(C═O)H |
| 3-3 | 3,5-Cl2 | O | H | — | CH2 | CH2 | CHNH(C═O)CH3 |
| 3-4 | 3,5-Cl2 | O | H | — | CH2 | CH2 | CHNH(C═O)C2H5 |
| 3-5 | 3,5-Cl2 | O | H | — | CH2 | CH2 | CHNH(C═O)CF3 |
| 3-6 | 3,5-Cl2 | O | H | — | CH2 | CH2 | CHNH(C═O)CH2CF3 |
| 3-7 | 3,5-Cl2 | O | H | — | CH2 | CH2 | CHNH(C═O)Pr-c |
| 3-8 | 3,5-Cl2 | O | H | — | CH2 | CH2 | CHNH(C═O)NHCH3 |
| 3-9 | 3,5-Cl2 | O | H | — | CH2 | CH2 | CHNH(C═O)NHC2H5 |
| 3-10 | 3,5-Cl2 | O | H | — | CH2 | CH2 | CHNH(C═O)NHPr-n |
| 3-11 | 3,5-Cl2 | O | H | — | CH2 | CH2 | CHNH(C═O)NHPr-i |
| 3-12 | 3,5-Cl2 | O | H | — | CH2 | CH2 | CHNH(C═O)NHPr-c |
| 3-13 | 3,5-Cl2 | O | H | — | CH2 | CH2 | CHNH(C═O)NHBu-n |
| 3-14 | 3,5-Cl2 | O | H | — | CH2 | CH2 | CHNH(C═O)NHBu-i |
| 3-15 | 3,5-Cl2 | O | H | — | CH2 | CH2 | CHNH(C═O)NHBu-s |
| 3-16 | 3,5-Cl2 | O | H | — | CH2 | CH2 | CHNH(C═O)NHBu-t |
| 3-17 | 3,5-Cl2 | O | H | — | CH2 | CH2 | CHNH(C═O)NHBu-c |
| 3-18 | 3,5-Cl2 | O | H | — | CH2 | CH2 | CHNH(C═O)NHC2H4Cl |
| 3-19 | 3,5-Cl2 | O | H | — | CH2 | CH2 | CHNH(C═O)NHCH2CF3 |
| 3-20 | 3,5-Cl2 | O | H | — | CH2 | CH2 | CHNH(C═O)NHCH2CHCH2 |
| 3-21 | 3,5-Cl2 | O | H | — | CH2 | CH2 | CHNH(C═O)NHCH2CCH |
| 3-22 | 3,5-Cl2 | O | H | — | CH2 | CH2 | CHNH(C═S)CH3 |
| 3-23 | 3,5-Cl2 | O | H | — | CH2 | CH2 | CHNH(C═S)NHCH3 |
| 3-24 | 3,5-Cl2 | O | H | — | CH2 | CH2 | CHNH(C═S)NHC2H5 |
| 3-25 | 3,5-Cl2 | O | H | — | CH2 | CH2 | CHNH(C═O)N(CH3)2 |
| 3-26 | 3,5-Cl2 | O | H | — | CH2 | CH2 | CHNHCO2CH3 |
| 3-27 | 3,5-Cl2 | O | H | — | CH2 | CH2 | CHNHCO2C4H9-tert |
| 3-28 | 3,5-Cl2 | O | H | — | CH2 | CH2 | CHNHSO2CH3 |
| 3-29 | 3,5-Cl2 | O | H | — | CH2 | CH2 | CHN(CH3)(C═O)CH3 |
| 3-30 | 3,5-Cl2 | O | H | — | CH2 | CH2 | CHN(2-Py)(C═O)NHCH3 |
| 3-31 | 3,5-Cl2 | O | H | — | CH2 | CH2 | CHN(CH3)(C═O)NHCH3 |
| 3-32 | 3,5-Cl2 | O | H | — | CH2 | CH2 | CHNH(C═O)NHC2H4OH |
| 3-33 | 3,5-Cl2 | O | H | — | CH2 | CH2 | CH(2-phthalimide) |
| 3-34 | 3,5-Cl2 | O | H | — | CH2 | CH2 | CHOH |
| 3-35 | 3,5-Cl2 | O | H | — | CH2 | CH2 | CHO(C═O)CH3 |
| 3-36 | 3,5-Cl2 | O | H | — | CH2 | CH2 | CHO(C═O)NHC2H5 |
| 3-37 | 3,4,5-Cl3 | O | H | — | CH2 | CH2 | CHNH2 |
| 3-38 | 3,4,5-Cl3 | O | H | — | CH2 | CH2 | CHNH(C═O)H |
| 3-39 | 3,4,5-Cl3 | O | H | — | CH2 | CH2 | CHNH(C═O)CH3 |
| 3-40 | 3,4,5-Cl3 | O | H | — | CH2 | CH2 | CHNH(C═O)C2H5 |
| 3-41 | 3,4,5-Cl3 | O | H | — | CH2 | CH2 | CHNH(C═O)CF3 |
| 3-42 | 3,4,5-Cl3 | O | H | — | CH2 | CH2 | CHNH(C═O)CH2CF3 |
| 3-43 | 3,4,5-Cl3 | O | H | — | CH2 | CH2 | CHNH(C═O)Pr-c |
| 3-44 | 3,4,5-Cl3 | O | H | — | CH2 | CH2 | CHNH(C═O)NHCH3 |
| 3-45 | 3,4,5-Cl3 | O | H | — | CH2 | CH2 | CHNH(C═O)NHC2H5 |
| 3-46 | 3,4,5-Cl3 | O | H | — | CH2 | CH2 | CHNH(C═O)NHPr-n |
| 3-47 | 3,4,5-Cl3 | O | H | — | CH2 | CH2 | CHNH(C═O)NHPr-i |
| 3-48 | 3,4,5-Cl3 | O | H | — | CH2 | CH2 | CHNH(C═O)NHPr-c |
| 3-49 | 3,4,5-Cl3 | O | H | — | CH2 | CH2 | CHNH(C═O)NHBu-n |
| 3-50 | 3,4,5-Cl3 | O | H | — | CH2 | CH2 | CHNH(C═O)NHBu-i |
| 3-51 | 3,4,5-Cl3 | O | H | — | CH2 | CH2 | CHNH(C═O)NHBu-s |
| 3-52 | 3,4,5-Cl3 | O | H | — | CH2 | CH2 | CHNH(C═O)NHBu-t |
| 3-53 | 3,4,5-Cl3 | O | H | — | CH2 | CH2 | CHNH(C═O)NHBu-c |
| 3-54 | 3,4,5-Cl3 | O | H | — | CH2 | CH2 | CHNH(C═O)NHC2H4Cl |
| 3-55 | 3,4,5-Cl3 | O | H | — | CH2 | CH2 | CHNH(C═O)NHCH2CF3 |
| 3-56 | 3,4,5-Cl3 | O | H | — | CH2 | CH2 | CHNH(C═O)NHCH2CHCH2 |
| 3-57 | 3,4,5-Cl3 | O | H | — | CH2 | CH2 | CHNH(C═O)NHCH2CCH |
| 3-58 | 3,4,5-Cl3 | O | H | — | CH2 | CH2 | CHNH(C═S)CH3 |
| 3-59 | 3,4,5-Cl3 | O | H | — | CH2 | CH2 | CHNH(C═S)NHCH3 |
| 3-60 | 3,4,5-Cl3 | O | H | — | CH2 | CH2 | CHNH(C═S)NHC2H5 |
| 3-61 | 3,4,5-Cl3 | O | H | — | CH2 | CH2 | CHNH(C═O)N(CH3)2 |
| 3-62 | 3,4,5-Cl3 | O | H | — | CH2 | CH2 | CHNHCO2CH3 |
| 3-63 | 3,4,5-Cl3 | O | H | — | CH2 | CH2 | CHNHCO2C4H9-tert |
| 3-64 | 3,4,5-Cl3 | O | H | — | CH2 | CH2 | CHNHSO2CH3 |
| 3-65 | 3,4,5-Cl3 | O | H | — | CH2 | CH2 | CHN(CH3)(C═O)CH3 |
| 3-66 | 3,4,5-Cl3 | O | H | — | CH2 | CH2 | CHN(2-Py)(C═O)NHCH3 |
| 3-67 | 3,4,5-Cl3 | O | H | — | CH2 | CH2 | CHN(CH3)(C═O)NHCH3 |
| 3-68 | 3,4,5-Cl3 | O | H | — | CH2 | CH2 | CH(2-phthalimide) |
| 3-69 | 3,4,5-Cl3 | O | H | — | CH2 | CH2 | CHOH |
| 3-70 | 3,4,5-Cl3 | O | H | — | CH2 | CH2 | CHO(C═O)CH3 |
| 3-71 | 3,4,5-Cl3 | O | H | — | CH2 | CH2 | CHO(C═O)NHC2H5 |
| 3-72 | 3,4-Cl2, 5-CF3 | O | H | — | CH2 | CH2 | CHNH(C═O)CH3 |
| 3-73 | 3,4-Cl2, 5-CF3 | O | H | — | CH2 | CH2 | CHNH(C═O)C2H5 |
| 3-74 | 3,4-Cl2, 5-CF3 | O | H | — | CH2 | CH2 | CHNH(C═O)CH2CF3 |
| 3-75 | 3,4-Cl2, 5-CF3 | O | H | — | CH2 | CH2 | CHNH(C═O)Pr-c |
| 3-76 | 3,4-Cl2, 5-CF3 | O | H | — | CH2 | CH2 | CHNH(C═O)NHCH3 |
| 3-77 | 3,4-Cl2, 5-CF3 | O | H | — | CH2 | CH2 | CHNH(C═O)NHC2H5 |
| 3-78 | 3,4-Cl2, 5-CF3 | O | H | — | CH2 | CH2 | CHNH(C═O)NHCH2CCH |
| 3-79 | 3,4-Cl2, 5-CF3 | O | H | — | CH2 | CH2 | CHNH(C═S)NHCH3 |
| 3-80 | 3,5-Br2 | O | H | — | CH2 | CH2 | CHNH(C═O)CH3 |
| 3-81 | 3,5-Br2 | O | H | — | CH2 | CH2 | CHNH(C═O)C2H5 |
| 3-82 | 3,5-Br2 | O | H | — | CH2 | CH2 | CHNH(C═O)CH2CF3 |
| 3-83 | 3,5-Br2 | O | H | — | CH2 | CH2 | CHNH(C═O)Pr-c |
| 3-84 | 3,5-Br2 | O | H | — | CH2 | CH2 | CHNH(C═O)NHCH3 |
| 3-85 | 3,5-Br2 | O | H | — | CH2 | CH2 | CHNH(C═O)NHC2H5 |
| 3-86 | 3,5-Br2 | O | H | — | CH2 | CH2 | CHNH(C═O)NHCH2CCH |
| 3-87 | 3,5-Br2 | O | H | — | CH2 | CH2 | CHNH(C═S)NHCH3 |
| 3-88 | 3,5-(CF3)2 | O | H | — | CH2 | CH2 | CHNH(C═O)CH3 |
| 3-89 | 3,5-(CF3)2 | O | H | — | CH2 | CH2 | CHNH(C═O)C2H5 |
| 3-90 | 3,5-(CF3)2 | O | H | — | CH2 | CH2 | CHNH(C═O)CH2CF3 |
| 3-91 | 3,5-(CF3)2 | O | H | — | CH2 | CH2 | CHNH(C═O)Pr-c |
| 3-92 | 3,5-(CF3)2 | O | H | — | CH2 | CH2 | CHNH(C═O)NHCH3 |
| 3-93 | 3,5-(CF3)2 | O | H | — | CH2 | CH2 | CHNH(C═O)NHC2H5 |
| 3-94 | 3,5-(CF3)2 | O | H | — | CH2 | CH2 | CHNH(C═O)NHCH2CCH |
| 3-95 | 3,5-(CF3)2 | O | H | — | CH2 | CH2 | CHNH(C═S)NHCH3 |
| 3-96 | 3-Cl, 5-CF3 | O | H | — | CH2 | CH2 | CHNH(C═O)CH3 |
| 3-97 | 3-Cl, 5-CF3 | O | H | — | CH2 | CH2 | CHNH(C═O)C2H5 |
| 3-98 | 3-Cl, 5-CF3 | O | H | — | CH2 | CH2 | CHNH(C═O)CH2CF3 |
| 3-99 | 3-Cl, 5-CF3 | O | H | — | CH2 | CH2 | CHNH(C═O)Pr-c |
| 3-100 | 3-Cl, 5-CF3 | O | H | — | CH2 | CH2 | CHNH(C═O)NHCH3 |
| 3-101 | 3-Cl, 5-CF3 | O | H | — | CH2 | CH2 | CHNH(C═O)NHC2H5 |
| 3-102 | 3-Cl, 5-CF3 | O | H | — | CH2 | CH2 | CHNH(C═O)NHCH2CCH |
| 3-103 | 3-Cl, 5-CF3 | O | H | — | CH2 | CH2 | CHNH(C═S)NHCH3 |
| 3-104 | 3,5-Cl2 | CH2 | H | — | CH2 | CH2 | CHNH2 |
| 3-105 | 3,5-Cl2 | CH2 | H | — | CH2 | CH2 | CHNH(C═O)H |
| 3-106 | 3,5-Cl2 | CH2 | H | — | CH2 | CH2 | CHNH(C═O)CH3 |
| 3-107 | 3,5-Cl2 | CH2 | H | — | CH2 | CH2 | CHNH(C═O)C2H5 |
| 3-108 | 3,5-Cl2 | CH2 | H | — | CH2 | CH2 | CHNH(C═O)CF3 |
| 3-109 | 3,5-Cl2 | CH2 | H | — | CH2 | CH2 | CHNH(C═O)CH2CF3 |
| 3-110 | 3,5-Cl2 | CH2 | H | — | CH2 | CH2 | CHNH(C═O)Pr-c |
| 3-111 | 3,5-Cl2 | CH2 | H | — | CH2 | CH2 | CHNH(C═O)NHCH3 |
| 3-112 | 3,5-Cl2 | CH2 | H | — | CH2 | CH2 | CHNH(C═O)NHC2H5 |
| 3-113 | 3,5-Cl2 | CH2 | H | — | CH2 | CH2 | CHNH(C═O)NHPr-n |
| 3-114 | 3,5-Cl2 | CH2 | H | — | CH2 | CH2 | CHNH(C═O)NHPr-i |
| 3-115 | 3,5-Cl2 | CH2 | H | — | CH2 | CH2 | CHNH(C═O)NHPr-c |
| 3-116 | 3,5-Cl2 | CH2 | H | — | CH2 | CH2 | CHNH(C═O)NHBu-n |
| 3-117 | 3,5-Cl2 | CH2 | H | — | CH2 | CH2 | CHNH(C═O)NHBu-i |
| 3-118 | 3,5-Cl2 | CH2 | H | — | CH2 | CH2 | CHNH(C═O)NHBu-s |
| 3-119 | 3,5-Cl2 | CH2 | H | — | CH2 | CH2 | CHNH(C═O)NHBu-t |
| 3-120 | 3,5-Cl2 | CH2 | H | — | CH2 | CH2 | CHNH(C═O)NHBu-c |
| 3-121 | 3,5-Cl2 | CH2 | H | — | CH2 | CH2 | CHNH(C═O)NHC2H4Cl |
| 3-122 | 3,5-Cl2 | CH2 | H | — | CH2 | CH2 | CHNH(C═O)NHCH2CF3 |
| 3-123 | 3,5-Cl2 | CH2 | H | — | CH2 | CH2 | CHNH(C═O)NHCH2CHCH2 |
| 3-124 | 3,5-Cl2 | CH2 | H | — | CH2 | CH2 | CHNH(C═O)NHCH2CCH |
| 3-125 | 3,5-Cl2 | CH2 | H | — | CH2 | CH2 | CHNH(C═S)CH3 |
| 3-126 | 3,5-Cl2 | CH2 | H | — | CH2 | CH2 | CHNH(C═S)NHCH3 |
| 3-127 | 3,5-Cl2 | CH2 | H | — | CH2 | CH2 | CHNH(C═S)NHC2H5 |
| 3-128 | 3,5-Cl2 | CH2 | H | — | CH2 | CH2 | CHNH(C═O)N(CH3)2 |
| 3-129 | 3,5-Cl2 | CH2 | H | — | CH2 | CH2 | CHNHCO2CH3 |
| 3-130 | 3,5-Cl2 | CH2 | H | — | CH2 | CH2 | CHNHCO2C4H9-tert |
| 3-131 | 3,5-Cl2 | CH2 | H | — | CH2 | CH2 | CHNHSO2CH3 |
| 3-132 | 3,5-Cl2 | CH2 | H | — | CH2 | CH2 | CHN(CH3)(C═O)CH3 |
| 3-133 | 3,5-Cl2 | CH2 | H | — | CH2 | CH2 | CHN(2-Py)(C═O)NHCH3 |
| 3-134 | 3,5-Cl2 | CH2 | H | — | CH2 | CH2 | CHN(CH3)(C═O)NHCH3 |
| 3-135 | 3,5-Cl2 | CH2 | H | — | CH2 | CH2 | CHNH(C═O)NHC2H4OH |
| 3-136 | 3,5-Cl2 | CH2 | H | — | CH2 | CH2 | CH(2-phthalimide) |
| 3-137 | 3,5-Cl2 | CH2 | H | — | CH2 | CH2 | CHOH |
| 3-138 | 3,5-Cl2 | CH2 | H | — | CH2 | CH2 | CHO(C═O)CH3 |
| 3-139 | 3,5-Cl2 | CH2 | H | — | CH2 | CH2 | CHO(C═O)NHC2H5 |
| 3-140 | 3,4,5-Cl3 | CH2 | H | — | CH2 | CH2 | CHNH2 |
| 3-141 | 3,4,5-Cl3 | CH2 | H | — | CH2 | CH2 | CHNH(C═O)H |
| 3-142 | 3,4,5-Cl3 | CH2 | H | — | CH2 | CH2 | CHNH(C═O)CH3 |
| 3-143 | 3,4,5-Cl3 | CH2 | H | — | CH2 | CH2 | CHNH(C═O)C2H5 |
| 3-144 | 3,4,5-Cl3 | CH2 | H | — | CH2 | CH2 | CHNH(C═O)CF3 |
| 3-145 | 3,4,5-Cl3 | CH2 | H | — | CH2 | CH2 | CHNH(C═O)CH2CF3 |
| 3-146 | 3,4,5-Cl3 | CH2 | H | — | CH2 | CH2 | CHNH(C═O)Pr-c |
| 3-147 | 3,4,5-Cl3 | CH2 | H | — | CH2 | CH2 | CHNH(C═O)NHCH3 |
| 3-148 | 3,4,5-Cl3 | CH2 | H | — | CH2 | CH2 | CHNH(C═O)NHC2H5 |
| 3-149 | 3,4,5-Cl3 | CH2 | H | — | CH2 | CH2 | CHNH(C═O)NHPr-n |
| 3-150 | 3,4,5-Cl3 | CH2 | H | — | CH2 | CH2 | CHNH(C═O)NHPr-i |
| 3-151 | 3,4,5-Cl3 | CH2 | H | — | CH2 | CH2 | CHNH(C═O)NHPr-c |
| 3-152 | 3,4,5-Cl3 | CH2 | H | — | CH2 | CH2 | CHNH(C═O)NHBu-n |
| 3-153 | 3,4,5-Cl3 | CH2 | H | — | CH2 | CH2 | CHNH(C═O)NHBu-i |
| 3-154 | 3,4,5-Cl3 | CH2 | H | — | CH2 | CH2 | CHNH(C═O)NHBu-s |
| 3-155 | 3,4,5-Cl3 | CH2 | H | — | CH2 | CH2 | CHNH(C═O)NHBu-t |
| 3-156 | 3,4,5-Cl3 | CH2 | H | — | CH2 | CH2 | CHNH(C═O)NHBu-c |
| 3-157 | 3,4,5-Cl3 | CH2 | H | — | CH2 | CH2 | CHNH(C═O)NHC2H4Cl |
| 3-158 | 3,4,5-Cl3 | CH2 | H | — | CH2 | CH2 | CHNH(C═O)NHCH2CF3 |
| 3-159 | 3,4,5-Cl3 | CH2 | H | — | CH2 | CH2 | CHNH(C═O)NHCH2CHCH2 |
| 3-160 | 3,4,5-Cl3 | CH2 | H | — | CH2 | CH2 | CHNH(C═O)NHCH2CCH |
| 3-161 | 3,4,5-Cl3 | CH2 | H | — | CH2 | CH2 | CHNH(C═S)CH3 |
| 3-162 | 3,4,5-Cl3 | CH2 | H | — | CH2 | CH2 | CHNH(C═S)NHCH3 |
| 3-163 | 3,4,5-Cl3 | CH2 | H | — | CH2 | CH2 | CHNH(C═S)NHC2H5 |
| 3-164 | 3,4,5-Cl3 | CH2 | H | — | CH2 | CH2 | CHNH(C═O)N(CH3)2 |
| 3-165 | 3,4,5-Cl3 | CH2 | H | — | CH2 | CH2 | CHNHCO2CH3 |
| 3-166 | 3,4,5-Cl3 | CH2 | H | — | CH2 | CH2 | CHNHCO2C4H9-tert |
| 3-167 | 3,4,5-Cl3 | CH2 | H | — | CH2 | CH2 | CHNHSO2CH3 |
| 3-168 | 3,4,5-Cl3 | CH2 | H | — | CH2 | CH2 | CHN(CH3)(C═O)CH3 |
| 3-169 | 3,4,5-Cl3 | CH2 | H | — | CH2 | CH2 | CHN(2-Py)(C═O)NHCH3 |
| 3-170 | 3,4,5-Cl3 | CH2 | H | — | CH2 | CH2 | CHN(CH3)(C═O)NHCH3 |
| 3-171 | 3,4,5-Cl3 | CH2 | H | — | CH2 | CH2 | CH(2-phthalimide) |
| 3-172 | 3,4-Cl2, 5-CF3 | CH2 | H | — | CH2 | CH2 | CHNH(C═O)CH3 |
| 3-173 | 3,4-Cl2, 5-CF3 | CH2 | H | — | CH2 | CH2 | CHNH(C═O)C2H5 |
| 3-174 | 3,4-Cl2, 5-CF3 | CH2 | H | — | CH2 | CH2 | CHNH(C═O)CH2CF3 |
| 3-175 | 3,4-Cl2, 5-CF3 | CH2 | H | — | CH2 | CH2 | CHNH(C═O)Pr-c |
| 3-176 | 3,4-Cl2, 5-CF3 | CH2 | H | — | CH2 | CH2 | CHNH(C═O)NHCH3 |
| 3-177 | 3,4-Cl2, 5-CF3 | CH2 | H | — | CH2 | CH2 | CHNH(C═O)NHC2H5 |
| 3-178 | 3,4-Cl2, 5-CF3 | CH2 | H | — | CH2 | CH2 | CHNH(C═O)NHCH2CCH |
| 3-179 | 3,4-Cl2, 5-CF3 | CH2 | H | — | CH2 | CH2 | CHNH(C═S)NHCH3 |
| 3-180 | 3,5-Br2 | CH2 | H | — | CH2 | CH2 | CHNH(C═O)CH3 |
| 3-181 | 3,5-Br2 | CH2 | H | — | CH2 | CH2 | CHNH(C═O)C2H5 |
| 3-182 | 3,5-Br2 | CH2 | H | — | CH2 | CH2 | CHNH(C═O)CH2CF3 |
| 3-183 | 3,5-Br2 | CH2 | H | — | CH2 | CH2 | CHNH(C═O)Pr-c |
| 3-184 | 3,5-Br2 | CH2 | H | — | CH2 | CH2 | CHNH(C═O)NHCH3 |
| 3-185 | 3,5-Br2 | CH2 | H | — | CH2 | CH2 | CHNH(C═O)NHC2H5 |
| 3-186 | 3,5-Br2 | CH2 | H | — | CH2 | CH2 | CHNH(C═O)NHCH2CCH |
| 3-187 | 3,5-Br2 | CH2 | H | — | CH2 | CH2 | CHNH(C═S)NHCH3 |
| 3-188 | 3,5-(CF3)2 | CH2 | H | — | CH2 | CH2 | CHNH(C═O)CH3 |
| 3-189 | 3,5-(CF3)2 | CH2 | H | — | CH2 | CH2 | CHNH(C═O)C2H5 |
| 3-190 | 3,5-(CF3)2 | CH2 | H | — | CH2 | CH2 | CHNH(C═O)CH2CF3 |
| 3-191 | 3,5-(CF3)2 | CH2 | H | — | CH2 | CH2 | CHNH(C═O)Pr-c |
| 3-192 | 3,5-(CF3)2 | CH2 | H | — | CH2 | CH2 | CHNH(C═O)NHCH3 |
| 3-193 | 3,5-(CF3)2 | CH2 | H | — | CH2 | CH2 | CHNH(C═O)NHC2H5 |
| 3-194 | 3,5-(CF3)2 | CH2 | H | — | CH2 | CH2 | CHNH(C═S)NHCH3 |
| 3-195 | 3-Cl, 5-CF3 | CH2 | H | — | CH2 | CH2 | CHNH(C═O)CH3 |
| 3-196 | 3-Cl, 5-CF3 | CH2 | H | — | CH2 | CH2 | CHNH(C═O)C2H5 |
| 3-197 | 3-Cl, 5-CF3 | CH2 | H | — | CH2 | CH2 | CHNH(C═O)CH2CF3 |
| 3-198 | 3-Cl, 5-CF3 | CH2 | H | — | CH2 | CH2 | CHNH(C═O)Pr-c |
| 3-199 | 3-Cl, 5-CF3 | CH2 | H | — | CH2 | CH2 | CHNH(C═O)NHCH3 |
| 3-200 | 3-Cl, 5-CF3 | CH2 | H | — | CH2 | CH2 | CHNH(C═O)NHC2H5 |
| 3-201 | 3-Cl, 5-CF3 | CH2 | H | — | CH2 | CH2 | CHNH(C═O)NHCH2CCH |
| 3-202 | 3-Cl, 5-CF3 | CH2 | H | — | CH2 | CH2 | CHNH(C═S)NHCH3 |
| 3-203 | 3,5-Cl2 | NCH3 | H | — | CH2 | CH2 | CHNH(C═O)CH3 |
| 3-204 | 3,5-Cl2 | NCH3 | H | — | CH2 | CH2 | CHNH(C═O)C2H5 |
| 3-205 | 3,5-Cl2 | NCH3 | H | — | CH2 | CH2 | CHNH(C═O)CH2CF3 |
| 3-206 | 3,5-Cl2 | NCH3 | H | — | CH2 | CH2 | CHNH(C═O)Pr-c |
| 3-207 | 3,5-Cl2 | NCH3 | H | — | CH2 | CH2 | CHNH(C═O)NHCH3 |
| 3-208 | 3,5-Cl2 | NCH3 | H | — | CH2 | CH2 | CHNH(C═O)NHC2H5 |
| 3-209 | 3,5-Cl2 | NCH3 | H | — | CH2 | CH2 | CHNH(C═O)NHCH2CCH |
| 3-210 | 3,5-Cl2 | NCH3 | H | — | CH2 | CH2 | CHNH(C═S)NHCH3 |
| 3-211 | 3,5-Cl2 | O | H | CH2 | CH2 | CH2 | CHNH2 |
| 3-212 | 3,5-Cl2 | O | H | CH2 | CH2 | CH2 | CHNH(C═O)H |
| 3-213 | 3,5-Cl2 | O | H | CH2 | CH2 | CH2 | CHNH(C═O)CH3 |
| 3-214 | 3,5-Cl2 | O | H | CH2 | CH2 | CH2 | CHNH(C═O)C2H5 |
| 3-215 | 3,5-Cl2 | O | H | CH2 | CH2 | CH2 | CHNH(C═O)CF3 |
| 3-216 | 3,5-Cl2 | O | H | CH2 | CH2 | CH2 | CHNH(C═O)CH2Cl |
| 3-217 | 3,5-Cl2 | O | H | CH2 | CH2 | CH2 | CHNH(C═O)CH2CF3 |
| 3-218 | 3,5-Cl2 | O | H | CH2 | CH2 | CH2 | CHNH(C═O)Pr-c |
| 3-219 | 3,5-Cl2 | O | H | CH2 | CH2 | CH2 | CHNH(C═O)Ph |
| 3-220 | 3,5-Cl2 | O | H | CH2 | CH2 | CH2 | CHNH(C═O)NHCH3 |
| 3-221 | 3,5-Cl2 | O | H | CH2 | CH2 | CH2 | CHNH(C═O)NHC2H5 |
| 3-222 | 3,5-Cl2 | O | H | CH2 | CH2 | CH2 | CHNH(C═O)NHPr-n |
| 3-223 | 3,5-Cl2 | O | H | CH2 | CH2 | CH2 | CHNH(C═O)NHPr-i |
| 3-224 | 3,5-Cl2 | O | H | CH2 | CH2 | CH2 | CHNH(C═O)NHPr-c |
| 3-225 | 3,5-Cl2 | O | H | CH2 | CH2 | CH2 | CHNH(C═O)NHBu-n |
| 3-226 | 3,5-Cl2 | O | H | CH2 | CH2 | CH2 | CHNH(C═O)NHBu-i |
| 3-227 | 3,5-Cl2 | O | H | CH2 | CH2 | CH2 | CHNH(C═O)NHBu-s |
| 3-228 | 3,5-Cl2 | O | H | CH2 | CH2 | CH2 | CHNH(C═O)NHBu-t |
| 3-229 | 3,5-Cl2 | O | H | CH2 | CH2 | CH2 | CHNH(C═O)NHBu-c |
| 3-230 | 3,5-Cl2 | O | H | CH2 | CH2 | CH2 | CHNH(C═O)NHC2H4Cl |
| 3-231 | 3,5-Cl2 | O | H | CH2 | CH2 | CH2 | CHNH(C═O)NHCH2CF3 |
| 3-232 | 3,5-Cl2 | O | H | CH2 | CH2 | CH2 | CHNH(C═O)NHCH2CHCH2 |
| 3-233 | 3,5-Cl2 | O | H | CH2 | CH2 | CH2 | CHNH(C═O)NHCH2CCH |
| 3-234 | 3,5-Cl2 | O | H | CH2 | CH2 | CH2 | CHNH(C═S)CH3 |
| 3-235 | 3,5-Cl2 | O | H | CH2 | CH2 | CH2 | CHNH(C═S)NHPr-c |
| 3-236 | 3,5-Cl2 | O | H | CH2 | CH2 | CH2 | CHNH(C═S)NHCH3 |
| 3-237 | 3,5-Cl2 | O | H | CH2 | CH2 | CH2 | CHNH(C═S)NHC2H5 |
| 3-238 | 3,5-Cl2 | O | H | CH2 | CH2 | CH2 | CHNH(C═O)N(CH3)2 |
| 3-239 | 3,5-Cl2 | O | H | CH2 | CH2 | CH2 | CHNHCO2CH3 |
| 3-240 | 3,5-Cl2 | O | H | CH2 | CH2 | CH2 | CHNHCO2C4H9-tert |
| 3-241 | 3,5-Cl2 | O | H | CH2 | CH2 | CH2 | CHNHSO2CH3 |
| 3-242 | 3,5-Cl2 | O | H | CH2 | CH2 | CH2 | CHN(CH3)(C═O)CH3 |
| 3-243 | 3,5-Cl2 | O | H | CH2 | CH2 | CH2 | CHN(2-Py)(C═O)NHCH3 |
| 3-244 | 3,5-Cl2 | O | H | CH2 | CH2 | CH2 | CHN(CH3)(C═O)NHCH3 |
| 3-245 | 3,5-Cl2 | O | H | CH2 | CH2 | CH2 | CHNH(C═O)NHC2H4OH |
| 3-246 | 3,5-Cl2 | O | H | CH2 | CH2 | CH2 | CHNH(2-oxazolyl) |
| 3-247 | 3,5-Cl2 | O | H | CH2 | CH2 | CH2 | CH(2-phthalimide) |
| 3-248 | 3,5-Cl2 | O | H | CH2 | CH2 | CH2 | CHOH |
| 3-249 | 3,5-Cl2 | O | H | CH2 | CH2 | CH2 | CHO(C═O)CH3 |
| 3-250 | 3,5-Cl2 | O | H | CH2 | CH2 | CH2 | CHO(C═O)NHC2H5 |
| 3-251 | 3,4,5-Cl3 | O | H | CH2 | CH2 | CH2 | CHNH2 |
| 3-252 | 3,4,5-Cl3 | O | H | CH2 | CH2 | CH2 | CHNH(C═O)H |
| 3-253 | 3,4,5-Cl3 | O | H | CH2 | CH2 | CH2 | CHNH(C═O)CH3 |
| 3-254 | 3,4,5-Cl3 | O | H | CH2 | CH2 | CH2 | CHNH(C═O)C2H5 |
| 3-255 | 3,4,5-Cl3 | O | H | CH2 | CH2 | CH2 | CHNH(C═O)CF3 |
| 3-256 | 3,4,5-Cl3 | O | H | CH2 | CH2 | CH2 | CHNH(C═O)CH2CF3 |
| 3-257 | 3,4,5-Cl3 | O | H | CH2 | CH2 | CH2 | CHNH(C═O)Pr-c |
| 3-258 | 3,4,5-Cl3 | O | H | CH2 | CH2 | CH2 | CHNH(C═O)NHCH3 |
| 3-259 | 3,4,5-Cl3 | O | H | CH2 | CH2 | CH2 | CHNH(C═O)NHC2H5 |
| 3-260 | 3,4,5-Cl3 | O | H | CH2 | CH2 | CH2 | CHNH(C═O)NHPr-n |
| 3-261 | 3,4,5-Cl3 | O | H | CH2 | CH2 | CH2 | CHNH(C═O)NHPr-i |
| 3-262 | 3,4,5-Cl3 | O | H | CH2 | CH2 | CH2 | CHNH(C═O)NHPr-c |
| 3-263 | 3,4,5-Cl3 | O | H | CH2 | CH2 | CH2 | CHNH(C═O)NHBu-n |
| 3-264 | 3,4,5-Cl3 | O | H | CH2 | CH2 | CH2 | CHNH(C═O)NHBu-i |
| 3-265 | 3,4,5-Cl3 | O | H | CH2 | CH2 | CH2 | CHNH(C═O)NHBu-s |
| 3-266 | 3,4,5-Cl3 | O | H | CH2 | CH2 | CH2 | CHNH(C═O)NHBu-t |
| 3-267 | 3,4,5-Cl3 | O | H | CH2 | CH2 | CH2 | CHNH(C═O)NHBu-c |
| 3-268 | 3,4,5-Cl3 | O | H | CH2 | CH2 | CH2 | CHNH(C═O)NHC2H4Cl |
| 3-269 | 3,4,5-Cl3 | O | H | CH2 | CH2 | CH2 | CHNH(C═O)NHCH2CF3 |
| 3-270 | 3,4,5-Cl3 | O | H | CH2 | CH2 | CH2 | CHNH(C═O)NHCH2CHCH2 |
| 3-271 | 3,4,5-Cl3 | O | H | CH2 | CH2 | CH2 | CHNH(C═O)NHCH2CCH |
| 3-272 | 3,4,5-Cl3 | O | H | CH2 | CH2 | CH2 | CHNH(C═S)CH3 |
| 3-273 | 3,4,5-Cl3 | O | H | CH2 | CH2 | CH2 | CHNH(C═S)NHCH3 |
| 3-274 | 3,4,5-Cl3 | O | H | CH2 | CH2 | CH2 | CHNH(C═S)NHC2H5 |
| 3-275 | 3,4,5-Cl3 | O | H | CH2 | CH2 | CH2 | CHNH(C═O)N(CH3)2 |
| 3-276 | 3,4,5-Cl3 | O | H | CH2 | CH2 | CH2 | CHNHCO2CH3 |
| 3-277 | 3,4,5-Cl3 | O | H | CH2 | CH2 | CH2 | CHNHCO2C4H9-tert |
| 3-278 | 3,4,5-Cl3 | O | H | CH2 | CH2 | CH2 | CHNHSO2CH3 |
| 3-279 | 3,4,5-Cl3 | O | H | CH2 | CH2 | CH2 | CHN(CH3)(C═O)CH3 |
| 3-280 | 3,4,5-Cl3 | O | H | CH2 | CH2 | CH2 | CHN(2-Py)(C═O)NHCH3 |
| 3-281 | 3,4,5-Cl3 | O | H | CH2 | CH2 | CH2 | CHN(CH3)(C═O)NHCH3 |
| 3-282 | 3,4,5-Cl3 | O | H | CH2 | CH2 | CH2 | CH(2-phthalimide) |
| 3-283 | 3,4,5-Cl3 | O | H | CH2 | CH2 | CH2 | CHOH |
| 3-284 | 3,4,5-Cl3 | O | H | CH2 | CH2 | CH2 | CHO(C═O)CH3 |
| 3-285 | 3,4,5-Cl3 | O | H | CH2 | CH2 | CH2 | CHO(C═O)NHC2H5 |
| 3-286 | 3,4-Cl2, 5-CF3 | O | H | CH2 | CH2 | CH2 | CHNH(C═O)CH3 |
| 3-287 | 3,4-Cl2, 5-CF3 | O | H | CH2 | CH2 | CH2 | CHNH(C═O)C2H5 |
| 3-288 | 3,4-Cl2, 5-CF3 | O | H | CH2 | CH2 | CH2 | CHNH(C═O)CH2CF3 |
| 3-289 | 3,4-Cl2, 5-CF3 | O | H | CH2 | CH2 | CH2 | CHNH(C═O)Pr-c |
| 3-290 | 3,4-Cl2, 5-CF3 | O | H | CH2 | CH2 | CH2 | CHNH(C═O)NHCH3 |
| 3-291 | 3,4-Cl2, 5-CF3 | O | H | CH2 | CH2 | CH2 | CHNH(C═O)NHC2H5 |
| 3-292 | 3,4-Cl2, 5-CF3 | O | H | CH2 | CH2 | CH2 | CHNH(C═O)NHCH2CCH |
| 3-293 | 3,4-Cl2, 5-CF3 | O | H | CH2 | CH2 | CH2 | CHNH(C═S)NHCH3 |
| 3-294 | 3,5-Br2 | O | H | CH2 | CH2 | CH2 | CHNH(C═O)CH3 |
| 3-295 | 3,5-Br2 | O | H | CH2 | CH2 | CH2 | CHNH(C═O)C2H5 |
| 3-296 | 3,5-Br2 | O | H | CH2 | CH2 | CH2 | CHNH(C═O)CH2CF3 |
| 3-297 | 3,5-Br2 | O | H | CH2 | CH2 | CH2 | CHNH(C═O)Pr-c |
| 3-298 | 3,5-Br2 | O | H | CH2 | CH2 | CH2 | CHNH(C═O)NHCH3 |
| 3-299 | 3,5-Br2 | O | H | CH2 | CH2 | CH2 | CHNH(C═O)NHC2H5 |
| 3-300 | 3,5-Br2 | O | H | CH2 | CH2 | CH2 | CHNH(C═O)NHCH2CCH |
| 3-301 | 3,5-Br2 | O | H | CH2 | CH2 | CH2 | CHNH(C═S)NHCH3 |
| 3-302 | 3,5-(CF3)2 | O | H | CH2 | CH2 | CH2 | CHNH(C═O)CH3 |
| 3-303 | 3,5-(CF3)2 | O | H | CH2 | CH2 | CH2 | CHNH(C═O)C2H5 |
| 3-304 | 3,5-(CF3)2 | O | H | CH2 | CH2 | CH2 | CHNH(C═O)CH2CF3 |
| 3-305 | 3,5-(CF3)2 | O | H | CH2 | CH2 | CH2 | CHNH(C═O)Pr-c |
| 3-306 | 3,5-(CF3)2 | O | H | CH2 | CH2 | CH2 | CHNH(C═O)NHCH3 |
| 3-307 | 3,5-(CF3)2 | O | H | CH2 | CH2 | CH2 | CHNH(C═O)NHC2H5 |
| 3-308 | 3,5-(CF3)2 | O | H | CH2 | CH2 | CH2 | CHNH(C═O)NHCH2CCH |
| 3-309 | 3,5-(CF3)2 | O | H | CH2 | CH2 | CH2 | CHNH(C═S)NHCH3 |
| 3-310 | 3-Cl, 5-CF3 | O | H | CH2 | CH2 | CH2 | CHNH(C═O)CH3 |
| 3-311 | 3-Cl, 5-CF3 | O | H | CH2 | CH2 | CH2 | CHNH(C═O)C2H5 |
| 3-312 | 3-Cl, 5-CF3 | O | H | CH2 | CH2 | CH2 | CHNH(C═O)CH2CF3 |
| 3-313 | 3-Cl, 5-CF3 | O | H | CH2 | CH2 | CH2 | CHNH(C═O)Pr-c |
| 3-314 | 3-Cl, 5-CF3 | O | H | CH2 | CH2 | CH2 | CHNH(C═O)NHCH3 |
| 3-315 | 3-Cl, 5-CF3 | O | H | CH2 | CH2 | CH2 | CHNH(C═O)NHC2H5 |
| 3-316 | 3-Cl, 5-CF3 | O | H | CH2 | CH2 | CH2 | CHNH(C═O)NHCH2CCH |
| 3-317 | 3-Cl, 5-CF3 | O | H | CH2 | CH2 | CH2 | CHNH(C═S)NHCH3 |
| 3-318 | 3,5-Cl2 | CH2 | H | CH2 | CH2 | CH2 | CHNH2 |
| 3-319 | 3,5-Cl2 | CH2 | H | CH2 | CH2 | CH2 | CHNH(C═O)H |
| 3-320 | 3,5-Cl2 | CH2 | H | CH2 | CH2 | CH2 | CHNH(C═O)CH3 |
| 3-321 | 3,5-Cl2 | CH2 | H | CH2 | CH2 | CH2 | CHNH(C═O)C2H5 |
| 3-322 | 3,5-Cl2 | CH2 | H | CH2 | CH2 | CH2 | CHNH(C═O)CF3 |
| 3-323 | 3,5-Cl2 | CH2 | H | CH2 | CH2 | CH2 | CHNH(C═O)CH2CF3 |
| 3-324 | 3,5-Cl2 | CH2 | H | CH2 | CH2 | CH2 | CHNH(C═O)Pr-c |
| 3-325 | 3,5-Cl2 | CH2 | H | CH2 | CH2 | CH2 | CHNH(C═O)NHCH3 |
| 3-326 | 3,5-Cl2 | CH2 | H | CH2 | CH2 | CH2 | CHNH(C═O)NHC2H5 |
| 3-327 | 3,5-Cl2 | CH2 | H | CH2 | CH2 | CH2 | CHNH(C═O)NHPr-n |
| 3-328 | 3,5-Cl2 | CH2 | H | CH2 | CH2 | CH2 | CHNH(C═O)NHPr-i |
| 3-329 | 3,5-Cl2 | CH2 | H | CH2 | CH2 | CH2 | CHNH(C═O)NHPr-c |
| 3-330 | 3,5-Cl2 | CH2 | H | CH2 | CH2 | CH2 | CHNH(C═O)NHBu-n |
| 3-331 | 3,5-Cl2 | CH2 | H | CH2 | CH2 | CH2 | CHNH(C═O)NHBu-i |
| 3-332 | 3,5-Cl2 | CH2 | H | CH2 | CH2 | CH2 | CHNH(C═O)NHBu-s |
| 3-333 | 3,5-Cl2 | CH2 | H | CH2 | CH2 | CH2 | CHNH(C═O)NHBu-t |
| 3-334 | 3,5-Cl2 | CH2 | H | CH2 | CH2 | CH2 | CHNH(C═O)NHBu-c |
| 3-335 | 3,5-Cl2 | CH2 | H | CH2 | CH2 | CH2 | CHNH(C═O)NHC2H4Cl |
| 3-336 | 3,5-Cl2 | CH2 | H | CH2 | CH2 | CH2 | CHNH(C═O)NHCH2CF3 |
| 3-337 | 3,5-Cl2 | CH2 | H | CH2 | CH2 | CH2 | CHNH(C═O)NHCH2CHCH2 |
| 3-338 | 3,5-Cl2 | CH2 | H | CH2 | CH2 | CH2 | CHNH(C═O)NHCH2CCH |
| 3-339 | 3,5-Cl2 | CH2 | H | CH2 | CH2 | CH2 | CHNH(C═S)CH3 |
| 3-340 | 3,5-Cl2 | CH2 | H | CH2 | CH2 | CH2 | CHNH(C═S)NHCH3 |
| 3-341 | 3,5-Cl2 | CH2 | H | CH2 | CH2 | CH2 | CHNH(C═S)NHC2H5 |
| 3-342 | 3,5-Cl2 | CH2 | H | CH2 | CH2 | CH2 | CHNH(C═O)N(CH3)2 |
| 3-343 | 3,5-Cl2 | CH2 | H | CH2 | CH2 | CH2 | CHNHCO2CH3 |
| 3-344 | 3,5-Cl2 | CH2 | H | CH2 | CH2 | CH2 | CHNHCO2C4H9-tert |
| 3-345 | 3,5-Cl2 | CH2 | H | CH2 | CH2 | CH2 | CHNHSO2CH3 |
| 3-346 | 3,5-Cl2 | CH2 | H | CH2 | CH2 | CH2 | CHN(CH3)(C═O)CH3 |
| 3-347 | 3,5-Cl2 | CH2 | H | CH2 | CH2 | CH2 | CHN(2-Py)(C═O)NHCH3 |
| 3-348 | 3,5-Cl2 | CH2 | H | CH2 | CH2 | CH2 | CHN(CH3)(C═O)NHCH3 |
| 3-349 | 3,5-Cl2 | CH2 | H | CH2 | CH2 | CH2 | CH(2-phthalimide) |
| 3-350 | 3,5-Cl2 | CH2 | H | CH2 | CH2 | CH2 | CHOH |
| 3-351 | 3,5-Cl2 | CH2 | H | CH2 | CH2 | CH2 | CHO(C═O)CH3 |
| 3-352 | 3,5-Cl2 | CH2 | H | CH2 | CH2 | CH2 | CHO(C═O)NHC2H5 |
| 3-353 | 3,4,5-Cl3 | CH2 | H | CH2 | CH2 | CH2 | CHNH2 |
| 3-354 | 3,4,5-Cl3 | CH2 | H | CH2 | CH2 | CH2 | CHNH(C═O)H |
| 3-355 | 3,4,5-Cl3 | CH2 | H | CH2 | CH2 | CH2 | CHNH(C═O)CH3 |
| 3-356 | 3,4,5-Cl3 | CH2 | H | CH2 | CH2 | CH2 | CHNH(C═O)C2H5 |
| 3-357 | 3,4,5-Cl3 | CH2 | H | CH2 | CH2 | CH2 | CHNH(C═O)CF3 |
| 3-358 | 3,4,5-Cl3 | CH2 | H | CH2 | CH2 | CH2 | CHNH(C═O)CH2CF3 |
| 3-359 | 3,4,5-Cl3 | CH2 | H | CH2 | CH2 | CH2 | CHNH(C═O)Pr-c |
| 3-360 | 3,4,5-Cl3 | CH2 | H | CH2 | CH2 | CH2 | CHNH(C═O)NHCH3 |
| 3-361 | 3,4,5-Cl3 | CH2 | H | CH2 | CH2 | CH2 | CHNH(C═O)NHC2H5 |
| 3-362 | 3,4,5-Cl3 | CH2 | H | CH2 | CH2 | CH2 | CHNH(C═O)NHPr-n |
| 3-363 | 3,4,5-Cl3 | CH2 | H | CH2 | CH2 | CH2 | CHNH(C═O)NHPr-i |
| 3-364 | 3,4,5-Cl3 | CH2 | H | CH2 | CH2 | CH2 | CHNH(C═O)NHPr-c |
| 3-365 | 3,4,5-Cl3 | CH2 | H | CH2 | CH2 | CH2 | CHNH(C═O)NHBu-n |
| 3-366 | 3,4,5-Cl3 | CH2 | H | CH2 | CH2 | CH2 | CHNH(C═O)NHBu-i |
| 3-367 | 3,4,5-Cl3 | CH2 | H | CH2 | CH2 | CH2 | CHNH(C═O)NHBu-s |
| 3-368 | 3,4,5-Cl3 | CH2 | H | CH2 | CH2 | CH2 | CHNH(C═O)NHBu-t |
| 3-369 | 3,4,5-Cl3 | CH2 | H | CH2 | CH2 | CH2 | CHNH(C═O)NHBu-c |
| 3-370 | 3,4,5-Cl3 | CH2 | H | CH2 | CH2 | CH2 | CHNH(C═O)NHC2H4Cl |
| 3-371 | 3,4,5-Cl3 | CH2 | H | CH2 | CH2 | CH2 | CHNH(C═O)NHCH2CF3 |
| 3-372 | 3,4,5-Cl3 | CH2 | H | CH2 | CH2 | CH2 | CHNH(C═O)NHCH2CHCH2 |
| 3-373 | 3,4,5-Cl3 | CH2 | H | CH2 | CH2 | CH2 | CHNH(C═O)NHCH2CCH |
| 3-374 | 3,4,5-Cl3 | CH2 | H | CH2 | CH2 | CH2 | CHNH(C═S)CH3 |
| 3-375 | 3,4,5-Cl3 | CH2 | H | CH2 | CH2 | CH2 | CHNH(C═S)NHCH3 |
| 3-376 | 3,4,5-Cl3 | CH2 | H | CH2 | CH2 | CH2 | CHNH(C═S)NHC2H5 |
| 3-377 | 3,4,5-Cl3 | CH2 | H | CH2 | CH2 | CH2 | CHNH(C═O)N(CH3)2 |
| 3-378 | 3,4,5-Cl3 | CH2 | H | CH2 | CH2 | CH2 | CHNHCO2CH3 |
| 3-379 | 3,4,5-Cl3 | CH2 | H | CH2 | CH2 | CH2 | CHNHCO2C4H9-tert |
| 3-380 | 3,4,5-Cl3 | CH2 | H | CH2 | CH2 | CH2 | CHNHSO2CH3 |
| 3-381 | 3,4,5-Cl3 | CH2 | H | CH2 | CH2 | CH2 | CHN(CH3)(C═O)CH3 |
| 3-382 | 3,4,5-Cl3 | CH2 | H | CH2 | CH2 | CH2 | CHN(2-Py)(C═O)NHCH3 |
| 3-383 | 3,4,5-Cl3 | CH2 | H | CH2 | CH2 | CH2 | CHN(CH3)(C═O)NHCH3 |
| 3-384 | 3,4,5-Cl3 | CH2 | H | CH2 | CH2 | CH2 | CH(2-phthalimide) |
| 3-385 | 3,4,5-Cl3 | CH2 | H | CH2 | CH2 | CH2 | CHOH |
| 3-386 | 3,4,5-Cl3 | CH2 | H | CH2 | CH2 | CH2 | CHO(C═O)CH3 |
| 3-387 | 3,4,5-Cl3 | CH2 | H | CH2 | CH2 | CH2 | CHO(C═O)NHC2H5 |
| 3-388 | 3,4-Cl2, 5-CF3 | CH2 | H | CH2 | CH2 | CH2 | CHNH(C═O)CH3 |
| 3-389 | 3,4-Cl2, 5-CF3 | CH2 | H | CH2 | CH2 | CH2 | CHNH(C═O)C2H5 |
| 3-390 | 3,4-Cl2, 5-CF3 | CH2 | H | CH2 | CH2 | CH2 | CHNH(C═O)CH2CF3 |
| 3-391 | 3,4-Cl2, 5-CF3 | CH2 | H | CH2 | CH2 | CH2 | CHNH(C═O)Pr-c |
| 3-392 | 3,4-Cl2, 5-CF3 | CH2 | H | CH2 | CH2 | CH2 | CHNH(C═O)NHCH3 |
| 3-393 | 3,4-Cl2, 5-CF3 | CH2 | H | CH2 | CH2 | CH2 | CHNH(C═O)NHC2H5 |
| 3-394 | 3,4-Cl2, 5-CF3 | CH2 | H | CH2 | CH2 | CH2 | CHNH(C═O)NHCH2CCH |
| 3-395 | 3,4-Cl2, 5-CF3 | CH2 | H | CH2 | CH2 | CH2 | CHNH(C═S)NHCH3 |
| 3-396 | 3,5-Br2 | CH2 | H | CH2 | CH2 | CH2 | CHNH(C═O)CH3 |
| 3-397 | 3,5-Br2 | CH2 | H | CH2 | CH2 | CH2 | CHNH(C═O)C2H5 |
| 3-398 | 3,5-Br2 | CH2 | H | CH2 | CH2 | CH2 | CHNH(C═O)CH2CF3 |
| 3-399 | 3,5-Br2 | CH2 | H | CH2 | CH2 | CH2 | CHNH(C═O)Pr-c |
| 3-400 | 3,5-Br2 | CH2 | H | CH2 | CH2 | CH2 | CHNH(C═O)NHCH3 |
| 3-401 | 3,5-Br2 | CH2 | H | CH2 | CH2 | CH2 | CHNH(C═O)NHC2H5 |
| 3-402 | 3,5-Br2 | CH2 | H | CH2 | CH2 | CH2 | CHNH(C═O)NHCH2CCH |
| 3-403 | 3,5-Br2 | CH2 | H | CH2 | CH2 | CH2 | CHNH(C═S)NHCH3 |
| 3-404 | 3,5-(CF3)2 | CH2 | H | CH2 | CH2 | CH2 | CHNH(C═O)CH3 |
| 3-405 | 3,5-(CF3)2 | CH2 | H | CH2 | CH2 | CH2 | CHNH(C═O)C2H5 |
| 3-406 | 3,5-(CF3)2 | CH2 | H | CH2 | CH2 | CH2 | CHNH(C═O)CH2CF3 |
| 3-407 | 3,5-(CF3)2 | CH2 | H | CH2 | CH2 | CH2 | CHNH(C═O)Pr-c |
| 3-408 | 3,5-(CF3)2 | CH2 | H | CH2 | CH2 | CH2 | CHNH(C═O)NHCH3 |
| 3-409 | 3,5-(CF3)2 | CH2 | H | CH2 | CH2 | CH2 | CHNH(C═O)NHC2H5 |
| 3-410 | 3,5-(CF3)2 | CH2 | H | CH2 | CH2 | CH2 | CHNH(C═O)NHCH2CCH |
| 3-411 | 3,5-(CF3)2 | CH2 | H | CH2 | CH2 | CH2 | CHNH(C═S)NHCH3 |
| 3-412 | 3-Cl, 5-CF3 | CH2 | H | CH2 | CH2 | CH2 | CHNH(C═O)CH3 |
| 3-413 | 3-Cl, 5-CF3 | CH2 | H | CH2 | CH2 | CH2 | CHNH(C═O)C2H5 |
| 3-414 | 3-Cl, 5-CF3 | CH2 | H | CH2 | CH2 | CH2 | CHNH(C═O)CH2CF3 |
| 3-415 | 3-Cl, 5-CF3 | CH2 | H | CH2 | CH2 | CH2 | CHNH(C═O)Pr-c |
| 3-416 | 3-Cl, 5-CF3 | CH2 | H | CH2 | CH2 | CH2 | CHNH(C═O)NHCH3 |
| 3-417 | 3-Cl, 5-CF3 | CH2 | H | CH2 | CH2 | CH2 | CHNH(C═O)NHC2H5 |
| 3-418 | 3-Cl, 5-CF3 | CH2 | H | CH2 | CH2 | CH2 | CHNH(C═O)NHCH2CCH |
| 3-419 | 3-Cl, 5-CF3 | CH2 | H | CH2 | CH2 | CH2 | CHNH(C═S)NHCH3 |
| 3-420 | 3,5-Cl2 | NCH3 | H | CH2 | CH2 | CH2 | CHNH(C═O)CH3 |
| 3-421 | 3,5-Cl2 | NCH3 | H | CH2 | CH2 | CH2 | CHNH(C═O)C2H5 |
| 3-422 | 3,5-Cl2 | NCH3 | H | CH2 | CH2 | CH2 | CHNH(C═O)CH2CF3 |
| 3-423 | 3,5-Cl2 | NCH3 | H | CH2 | CH2 | CH2 | CHNH(C═O)Pr-c |
| 3-424 | 3,5-Cl2 | NCH3 | H | CH2 | CH2 | CH2 | CHNH(C═O)NHCH3 |
| 3-425 | 3,5-Cl2 | NCH3 | H | CH2 | CH2 | CH2 | CHNH(C═O)NHC2H5 |
| 3-426 | 3,5-Cl2 | NCH3 | H | CH2 | CH2 | CH2 | CHNH(C═O)NHCH2CCH |
| 3-427 | 3,5-Cl2 | NCH3 | H | CH2 | CH2 | CH2 | CHNH(C═S)NHCH3 |
| 3-428 | 3,5-Cl2 | O | H | — | CH2 | CHNH2 | CH2 |
| 3-429 | 3,5-Cl2 | O | H | — | CH2 | CHNH(C═O)CH3 | CH2 |
| 3-430 | 3,5-Cl2 | O | H | — | CH2 | CHNH(C═O)C2H5 | CH2 |
| 3-431 | 3,5-Cl2 | O | H | — | CH2 | CHNH(C═O)CH2CF3 | CH2 |
| 3-432 | 3,5-Cl2 | O | H | — | CH2 | CHNH(C═O)Pr-c | CH2 |
| 3-433 | 3,5-Cl2 | O | H | — | CH2 | CHNH(C═O)NHCH3 | CH2 |
| 3-434 | 3,5-Cl2 | O | H | — | CH2 | CHNH(C═O)NHC2H5 | CH2 |
| 3-435 | 3,5-Cl2 | O | H | — | CH2 | CHNH(C═O)NHPr-c | CH2 |
| 3-436 | 3,5-Cl2 | O | H | — | CH2 | CHNH(C═O)NHCH2CCH | CH2 |
| 3-437 | 3,5-Cl2 | O | H | CH2 | CH2 | CHNH2 | CH2 |
| 3-438 | 3,5-Cl2 | O | H | CH2 | CH2 | CHNH(C═O)CH3 | CH2 |
| 3-439 | 3,5-Cl2 | O | H | CH2 | CH2 | CHNH(C═O)C2H5 | CH2 |
| 3-440 | 3,5-Cl2 | O | H | CH2 | CH2 | CHNH(C═O)CH2CF3 | CH2 |
| 3-441 | 3,5-Cl2 | O | H | CH2 | CH2 | CHNH(C═O)Pr-c | CH2 |
| 3-442 | 3,5-Cl2 | O | H | CH2 | CH2 | CHNH(C═O)NHCH3 | CH2 |
| 3-443 | 3,5-Cl2 | O | H | CH2 | CH2 | CHNH(C═O)NHC2H5 | CH2 |
| 3-444 | 3,5-Cl2 | O | H | CH2 | CH2 | CHNH(C═O)NHPr-c | CH2 |
| 3-445 | 3,5-Cl2 | O | H | CH2 | CH2 | CHNH(C═O)NHCH2CCH | CH2 |
| 3-446 | 3,5-Cl2 | CH2 | H | — | CH2 | CHNH2 | CH2 |
| 3-447 | 3,5-Cl2 | CH2 | H | — | CH2 | CHNH(C═O)CH3 | CH2 |
| 3-448 | 3,5-Cl2 | CH2 | H | — | CH2 | CHNH(C═O)C2H5 | CH2 |
| 3-449 | 3,5-Cl2 | CH2 | H | — | CH2 | CHNH(C═O)CH2CF3 | CH2 |
| 3-450 | 3,5-Cl2 | CH2 | H | — | CH2 | CHNH(C═O)Pr-c | CH2 |
| 3-451 | 3,5-Cl2 | CH2 | H | — | CH2 | CHNH(C═O)NHCH3 | CH2 |
| 3-452 | 3,5-Cl2 | CH2 | H | — | CH2 | CHNH(C═O)NHC2H5 | CH2 |
| 3-453 | 3,5-Cl2 | CH2 | H | — | CH2 | CHNH(C═O)NHPr-c | CH2 |
| 3-454 | 3,5-Cl2 | CH2 | H | — | CH2 | CHNH(C═O)NHCH2CCH | CH2 |
| 3-455 | 3,5-Cl2 | CH2 | H | CH2 | CH2 | CHNH2 | CH2 |
| 3-456 | 3,5-Cl2 | CH2 | H | CH2 | CH2 | CHNH(C═O)CH3 | CH2 |
| 3-457 | 3,5-Cl2 | CH2 | H | CH2 | CH2 | CHNH(C═O)C2H5 | CH2 |
| 3-458 | 3,5-Cl2 | CH2 | H | CH2 | CH2 | CHNH(C═O)CH2CF3 | CH2 |
| 3-459 | 3,5-Cl2 | CH2 | H | CH2 | CH2 | CHNH(C═O)Pr-c | CH2 |
| 3-460 | 3,5-Cl2 | CH2 | H | CH2 | CH2 | CHNH(C═O)NHCH3 | CH2 |
| 3-461 | 3,5-Cl2 | CH2 | H | CH2 | CH2 | CHNH(C═O)NHC2H5 | CH2 |
| 3-462 | 3,5-Cl2 | CH2 | H | CH2 | CH2 | CHNH(C═O)NHPr-c | CH2 |
| 3-463 | 3,5-Cl2 | CH2 | H | CH2 | CH2 | CHNH(C═O)NHCH2CCH | CH2 |
| 3-464 | 3,4,5-Cl3 | O | H | — | CH2 | CHNH2 | CH2 |
| 3-465 | 3,4,5-Cl3 | O | H | — | CH2 | CHNH(C═O)CH3 | CH2 |
| 3-466 | 3,4,5-Cl3 | O | H | — | CH2 | CHNH(C═O)C2H5 | CH2 |
| 3-467 | 3,4,5-Cl3 | O | H | — | CH2 | CHNH(C═O)CH2CF3 | CH2 |
| 3-468 | 3,4,5-Cl3 | O | H | — | CH2 | CHNH(C═O)Pr-c | CH2 |
| 3-469 | 3,4,5-Cl3 | O | H | — | CH2 | CHNH(C═O)NHCH3 | CH2 |
| 3-470 | 3,4,5-Cl3 | O | H | — | CH2 | CHNH(C═O)NHC2H5 | CH2 |
| 3-471 | 3,4,5-Cl3 | O | H | — | CH2 | CHNH(C═O)NHPr-c | CH2 |
| 3-472 | 3,4,5-Cl3 | O | H | — | CH2 | CHNH(C═O)NHCH2CCH | CH2 |
| 3-473 | 3,5-(CF3)2 | O | H | CH2 | CH2 | CHNH2 | CH2 |
| 3-474 | 3,5-(CF3)2 | O | H | CH2 | CH2 | CHNH(C═O)CH3 | CH2 |
| 3-475 | 3,5-(CF3)2 | O | H | CH2 | CH2 | CHNH(C═O)C2H5 | CH2 |
| 3-476 | 3,5-(CF3)2 | O | H | CH2 | CH2 | CHNH(C═O)CH2CF3 | CH2 |
| 3-477 | 3,5-(CF3)2 | O | H | CH2 | CH2 | CHNH(C═O)Pr-c | CH2 |
| 3-478 | 3,5-(CF3)2 | O | H | CH2 | CH2 | CHNH(C═O)NHCH3 | CH2 |
| 3-479 | 3,5-(CF3)2 | O | H | CH2 | CH2 | CHNH(C═O)NHC2H5 | CH2 |
| 3-480 | 3,5-(CF3)2 | O | H | CH2 | CH2 | CHNH(C═O)NHPr-c | CH2 |
| 3-481 | 3,5-(CF3)2 | O | H | CH2 | CH2 | CHNH(C═O)NHCH2CCH | CH2 |
| 3-482 | 3,4,5-Cl3 | CH2 | H | — | CH2 | CHNH2 | CH2 |
| 3-483 | 3,4,5-Cl3 | CH2 | H | — | CH2 | CHNH(C═O)CH3 | CH2 |
| 3-484 | 3,4,5-Cl3 | CH2 | H | — | CH2 | CHNH(C═O)C2H5 | CH2 |
| 3-485 | 3,4,5-Cl3 | CH2 | H | — | CH2 | CHNH(C═O)CH2CF3 | CH2 |
| 3-486 | 3,4,5-Cl3 | CH2 | H | — | CH2 | CHNH(C═O)Pr-c | CH2 |
| 3-487 | 3,4,5-Cl3 | CH2 | H | — | CH2 | CHNH(C═O)NHCH3 | CH2 |
| 3-488 | 3,4,5-Cl3 | CH2 | H | — | CH2 | CHNH(C═O)NHC2H5 | CH2 |
| 3-489 | 3,4,5-Cl3 | CH2 | H | — | CH2 | CHNH(C═O)NHPr-c | CH2 |
| 3-490 | 3,4,5-Cl3 | CH2 | H | — | CH2 | CHNH(C═O)NHCH2CCH | CH2 |
| 3-491 | 3,5-(CF3)2 | CH2 | H | CH2 | CH2 | CHNH2 | CH2 |
| 3-492 | 3,5-(CF3)2 | CH2 | H | CH2 | CH2 | CHNH(C═O)CH3 | CH2 |
| 3-493 | 3,5-(CF3)2 | CH2 | H | CH2 | CH2 | CHNH(C═O)C2H5 | CH2 |
| 3-494 | 3,5-(CF3)2 | CH2 | H | CH2 | CH2 | CHNH(C═O)CH2CF3 | CH2 |
| 3-495 | 3,5-(CF3)2 | CH2 | H | CH2 | CH2 | CHNH(C═O)Pr-c | CH2 |
| 3-496 | 3,5-(CF3)2 | CH2 | H | CH2 | CH2 | CHNH(C═O)NHCH3 | CH2 |
| 3-497 | 3,5-(CF3)2 | CH2 | H | CH2 | CH2 | CHNH(C═O)NHC2H5 | CH2 |
| 3-498 | 3,5-(CF3)2 | CH2 | H | CH2 | CH2 | CHNH(C═O)NHPr-c | CH2 |
| 3-499 | 3,5-(CF3)2 | CH2 | H | CH2 | CH2 | CHNH(C═O)NHCH2CCH | CH2 |
| 3-500 | 3,5-Cl2 | O | H | CH | CH | CH | CNH2 |
| 3-501 | 3,5-Cl2 | O | H | CH | CH | CH | CNH(C═O)CH3 |
| 3-502 | 3,5-Cl2 | O | H | CH | CH | CH | CNH(C═O)C2H5 |
| 3-503 | 3,5-Cl2 | O | H | CH | CH | CH | CNH(C═O)CH2CF3 |
| 3-504 | 3,5-Cl2 | O | H | CH | CH | CH | CNH(C═O)Pr-c |
| 3-505 | 3,5-Cl2 | O | H | CH | CH | CH | CNH(C═O)NHCH3 |
| 3-506 | 3,5-Cl2 | O | H | CH | CH | CH | CNH(C═O)NHC2H5 |
| 3-507 | 3,5-Cl2 | O | H | CH | CH | CH | CNH(C═O)NHPr-c |
| 3-508 | 3,5-Cl2 | O | H | CH | CH | CH | CNH(C═O)NHCH2CCH |
| 3-509 | 3,5-(CF3)2 | O | H | CH | CH | CH | CNH2 |
| 3-509 | 3,5-(CF3)2 | O | H | CH | CH | CH | CNH(C═O)CH3 |
| 3-511 | 3,5-(CF3)2 | O | H | CH | CH | CH | CNH(C═O)C2H5 |
| 3-512 | 3,5-(CF3)2 | O | H | CH | CH | CH | CNH(C═O)CH2CF3 |
| 3-513 | 3,5-(CF3)2 | O | H | CH | CH | CH | CNH(C═O)Pr-c |
| 3-514 | 3,5-(CF3)2 | O | H | CH | CH | CH | CNH(C═O)NHCH3 |
| 3-515 | 3,5-(CF3)2 | O | H | CH | CH | CH | CNH(C═O)NHC2H5 |
| 3-516 | 3,5-(CF3)2 | O | H | CH | CH | CH | CNH(C═O)NHPr-c |
| 3-517 | 3,5-(CF3)2 | O | H | CH | CH | CH | CNH(C═O)NHCH2CCH |
| 3-518 | 3,4,5-Cl3 | O | H | CH | CH | CH | CNH2 |
| 3-519 | 3,4,5-Cl3 | O | H | CH | CH | CH | CNH(C═O)CH3 |
| 3-520 | 3,4,5-Cl3 | O | H | CH | CH | CH | CNH(C═O)C2H5 |
| 3-521 | 3,4,5-Cl3 | O | H | CH | CH | CH | CNH(C═O)CH2CF3 |
| 3-522 | 3,4,5-Cl3 | O | H | CH | CH | CH | CNH(C═O)Pr-c |
| 3-523 | 3,4,5-Cl3 | O | H | CH | CH | CH | CNH(C═O)NHCH3 |
| 3-524 | 3,4,5-Cl3 | O | H | CH | CH | CH | CNH(C═O)NHC2H5 |
| 3-525 | 3,4,5-Cl3 | O | H | CH | CH | CH | CNH(C═O)NHPr-c |
| 3-526 | 3,4,5-Cl3 | O | H | CH | CH | CH | CNH(C═O)NHCH2CCH |
| 3-527 | 3,5-Cl2 | CH2 | H | CH | CH | CH | CNH2 |
| 3-528 | 3,5-Cl2 | CH2 | H | CH | CH | CH | CNH(C═O)CH3 |
| 3-529 | 3,5-Cl2 | CH2 | H | CH | CH | CH | CNH(C═O)C2H5 |
| 3-530 | 3,5-Cl2 | CH2 | H | CH | CH | CH | CNH(C═O)CH2CF3 |
| 3-531 | 3,5-Cl2 | CH2 | H | CH | CH | CH | CNH(C═O)Pr-c |
| 3-532 | 3,5-Cl2 | CH2 | H | CH | CH | CH | CNH(C═O)NHCH3 |
| 3-533 | 3,5-Cl2 | CH2 | H | CH | CH | CH | CNH(C═O)NHC2H5 |
| 3-534 | 3,5-Cl2 | CH2 | H | CH | CH | CH | CNH(C═O)NHPr-c |
| 3-535 | 3,5-Cl2 | CH2 | H | CH | CH | CH | CNH(C═O)NHCH2CCH |
| 3-536 | 3,5-(CF3)2 | CH2 | H | CH | CH | CH | CNH2 |
| 3-537 | 3,5-(CF3)2 | CH2 | H | CH | CH | CH | CNH(C═O)CH3 |
| 3-538 | 3,5-(CF3)2 | CH2 | H | CH | CH | CH | CNH(C═O)C2H5 |
| 3-539 | 3,5-(CF3)2 | CH2 | H | CH | CH | CH | CNH(C═O)CH2CF3 |
| 3-540 | 3,5-(CF3)2 | CH2 | H | CH | CH | CH | CNH(C═O)Pr-c |
| 3-541 | 3,5-(CF3)2 | CH2 | H | CH | CH | CH | CNH(C═O)NHCH3 |
| 3-542 | 3,5-(CF3)2 | CH2 | H | CH | CH | CH | CNH(C═O)NHC2H5 |
| 3-543 | 3,5-(CF3)2 | CH2 | H | CH | CH | CH | CNH(C═O)NHPr-c |
| 3-544 | 3,5-(CF3)2 | CH2 | H | CH | CH | CH | CNH(C═O)NHCH2CCH |
| 3-545 | 3,4,5-Cl3 | CH2 | H | CH | CH | CH | CNH2 |
| 3-546 | 3,4,5-Cl3 | CH2 | H | CH | CH | CH | CNH(C═O)CH3 |
| 3-547 | 3,4,5-Cl3 | CH2 | H | CH | CH | CH | CNH(C═O)C2H5 |
| 3-548 | 3,4,5-Cl3 | CH2 | H | CH | CH | CH | CNH(C═O)CH2CF3 |
| 3-549 | 3,4,5-Cl3 | CH2 | H | CH | CH | CH | CNH(C═O)Pr-c |
| 3-550 | 3,4,5-Cl3 | CH2 | H | CH | CH | CH | CNH(C═O)NHCH3 |
| 3-551 | 3,4,5-Cl3 | CH2 | H | CH | CH | CH | CNH(C═O)NHC2H5 |
| 3-552 | 3,4,5-Cl3 | CH2 | H | CH | CH | CH | CNH(C═O)NHPr-c |
| 3-553 | 3,4,5-Cl3 | CH2 | H | CH | CH | CH | CNH(C═O)NHCH2CCH |
| 3-554 | 3,5-Cl2 | CH2 | H | CH | CH | CNH2 | CH |
| 3-555 | 3,5-Cl2 | CH2 | H | CH | CH | CNH(C═O)CH3 | CH |
| 3-556 | 3,5-Cl2 | CH2 | H | CH | CH | CNH(C═O)C2H5 | CH |
| 3-557 | 3,5-Cl2 | CH2 | H | CH | CH | CNH(C═O)CH2CF3 | CH |
| 3-558 | 3,5-Cl2 | CH2 | H | CH | CH | CNH(C═O)Pr-c | CH |
| 3-559 | 3,5-Cl2 | CH2 | H | CH | CH | CNH(C═O)NHCH3 | CH |
| 3-560 | 3,5-Cl2 | CH2 | H | CH | CH | CNH(C═O)NHC2H5 | CH |
| 3-561 | 3,5-Cl2 | CH2 | H | CH | CH | CNH(C═O)NHPr-c | CH |
| 3-562 | 3,5-Cl2 | CH2 | H | CH | CH | CNH(C═O)NHCH2CCH | CH |
| 3-563 | 3,5-Cl2 | O | H | — | CH | CH | CHNH2 |
| 3-564 | 3,5-Cl2 | O | H | — | CH | CH | CHNH(C═O)CH3 |
| 3-565 | 3,5-Cl2 | O | H | — | CH | CH | CHNH(C═O)C2H5 |
| 3-566 | 3,5-Cl2 | O | H | — | CH | CH | CHNH(C═O)CH2CF3 |
| 3-567 | 3,5-Cl2 | O | H | — | CH | CH | CHNH(C═O)Pr-c |
| 3-568 | 3,5-Cl2 | O | H | — | CH | CH | CHNH(C═O)NHCH3 |
| 3-569 | 3,5-Cl2 | O | H | — | CH | CH | CHNH(C═O)NHC2H5 |
| 3-570 | 3,5-Cl2 | O | H | — | CH | CH | CHNH(C═O)NHPr-c |
| 3-571 | 3,5-Cl2 | O | H | — | CH | CH | CHNH(C═O)NHCH2CCH |
| 3-572 | 3,5-Cl2 | CH2 | H | — | CH | CH | CHNH2 |
| 3-573 | 3,5-Cl2 | CH2 | H | — | CH | CH | CHNH(C═O)CH3 |
| 3-574 | 3,5-Cl2 | CH2 | H | — | CH | CH | CHNH(C═O)C2H5 |
| 3-575 | 3,5-Cl2 | CH2 | H | — | CH | CH | CHNH(C═O)CH2CF3 |
| 3-576 | 3,5-Cl2 | CH2 | H | — | CH | CH | CHNH(C═O)Pr-c |
| 3-577 | 3,5-Cl2 | CH2 | H | — | CH | CH | CHNH(C═O)NHCH3 |
| 3-578 | 3,5-Cl2 | CH2 | H | — | CH | CH | CHNH(C═O)NHC2H5 |
| 3-579 | 3,5-Cl2 | CH2 | H | — | CH | CH | CHNH(C═O)NHPr-c |
| 3-580 | 3,5-Cl2 | CH2 | H | — | CH | CH | CHNH(C═O)NHCH2CCH |
| 3-581 | 3,5-(CF3)2 | O | H | — | CH | CH | CHNH2 |
| 3-582 | 3,5-(CF3)2 | O | H | — | CH | CH | CHNH(C═O)CH3 |
| 3-583 | 3,5-(CF3)2 | O | H | — | CH | CH | CHNH(C═O)C2H5 |
| 3-584 | 3,5-(CF3)2 | O | H | — | CH | CH | CHNH(C═O)CH2CF3 |
| 3-585 | 3,5-(CF3)2 | O | H | — | CH | CH | CHNH(C═O)Pr-c |
| 3-586 | 3,5-(CF3)2 | O | H | — | CH | CH | CHNH(C═O)NHCH3 |
| 3-587 | 3,5-(CF3)2 | O | H | — | CH | CH | CHNH(C═O)NHC2H5 |
| 3-588 | 3,5-(CF3)2 | O | H | — | CH | CH | CHNH(C═O)NHPr-c |
| 3-589 | 3,5-(CF3)2 | O | H | — | CH | CH | CHNH(C═O)NHCH2CCH |
| 3-590 | 3,5-Cl2 | O | H | — | CH2 | N | CNH2 |
| 3-591 | 3,5-Cl2 | O | H | — | CH2 | N | CNH(C═O)CH3 |
| 3-592 | 3,5-Cl2 | O | H | — | CH2 | N | CNH(C═O)C2H5 |
| 3-593 | 3,5-Cl2 | O | H | — | CH2 | N | CNH(C═O)CH2CF3 |
| 3-594 | 3,5-Cl2 | O | H | — | CH2 | N | CNH(C═O)Pr-c |
| 3-595 | 3,5-Cl2 | O | H | — | CH2 | N | CNH(C═O)NHCH3 |
| 3-596 | 3,5-Cl2 | O | H | — | CH2 | N | CNH(C═O)NHC2H5 |
| 3-597 | 3,5-Cl2 | O | H | — | CH2 | N | CNH(C═O)NHPr-c |
| 3-598 | 3,5-Cl2 | O | H | — | CH2 | N | CNH(C═O)NHCH2CCH |
| 3-599 | 3,5-Cl2 | O | H | CH2 | CH2 | N | CNH2 |
| 3-600 | 3,5-Cl2 | O | H | CH2 | CH2 | N | CNH(C═O)CH3 |
| 3-601 | 3,5-Cl2 | O | H | CH2 | CH2 | N | CNH(C═O)C2H5 |
| 3-602 | 3,5-Cl2 | O | H | CH2 | CH2 | N | CNH(C═O)CH2CF3 |
| 3-603 | 3,5-Cl2 | O | H | CH2 | CH2 | N | CNH(C═O)Pr-c |
| 3-604 | 3,5-Cl2 | O | H | CH2 | CH2 | N | CNH(C═O)NHCH3 |
| 3-605 | 3,5-Cl2 | O | H | CH2 | CH2 | N | CNH(C═O)NHC2H5 |
| 3-606 | 3,5-Cl2 | O | H | CH2 | CH2 | N | CNH(C═O)NHPr-c |
| 3-607 | 3,5-Cl2 | O | H | CH2 | CH2 | N | CNH(C═O)NHCH2CCH |
| 3-608 | 3,5-Cl2 | O | H | CH | CH | N | CNH2 |
| 3-609 | 3,5-Cl2 | O | H | CH | CH | N | CNH(C═O)CH3 |
| 3-610 | 3,5-Cl2 | O | H | CH | CH | N | CNH(C═O)C2H5 |
| 3-611 | 3,5-Cl2 | O | H | CH | CH | N | CNH(C═O)CH2CF3 |
| 3-612 | 3,5-Cl2 | O | H | CH | CH | N | CNH(C═O)Pr-c |
| 3-613 | 3,5-Cl2 | O | H | CH | CH | N | CNH(C═O)NHCH3 |
| 3-614 | 3,5-Cl2 | O | H | CH | CH | N | CNH(C═O)NHC2H5 |
| 3-615 | 3,5-Cl2 | O | H | CH | CH | N | CNH(C═O)NHPr-c |
| 3-616 | 3,5-Cl2 | O | H | CH | CH | N | CNH(C═O)NHCH2CCH |
| 3-617 | 3,5-Cl2 | O | H | CH | N | N | CNH2 |
| 3-618 | 3,5-Cl2 | O | H | CH | N | N | CNH(C═O)CH3 |
| 3-619 | 3,5-Cl2 | O | H | CH | N | N | CNH(C═O)C2H5 |
| 3-620 | 3,5-Cl2 | O | H | CH | N | N | CNH(C═O)CH2CF3 |
| 3-621 | 3,5-Cl2 | O | H | CH | N | N | CNH(C═O)Pr-c |
| 3-622 | 3,5-Cl2 | O | H | CH | N | N | CNH(C═O)NHCH3 |
| 3-623 | 3,5-Cl2 | O | H | CH | N | N | CNH(C═O)NHC2H5 |
| 3-624 | 3,5-Cl2 | O | H | CH | N | N | CNH(C═O)NHPr-c |
| 3-625 | 3,5-Cl2 | O | H | CH | N | N | CNH(C═O)NHCH2CCH |
| 3-626 | 3,5-Cl2 | O | H | CH | CH | CNH2 | N |
| 3-627 | 3,5-Cl2 | O | H | CH | CH | CNH(C═O)CH3 | N |
| 3-628 | 3,5-Cl2 | O | H | CH | CH | CNH(C═O)C2H5 | N |
| 3-629 | 3,5-Cl2 | O | H | CH | CH | CNH(C═O)CH2CF3 | N |
| 3-630 | 3,5-Cl2 | O | H | CH | CH | CNH(C═O)Pr-c | N |
| 3-631 | 3,5-Cl2 | O | H | CH | CH | CNH(C═O)NHCH3 | N |
| 3-632 | 3,5-Cl2 | O | H | CH | CH | CNH(C═O)NHC2H5 | N |
| 3-633 | 3,5-Cl2 | O | H | CH | CH | CNH(C═O)NHPr-c | N |
| 3-634 | 3,5-Cl2 | O | H | CH | CH | CNH(C═O)NHCH2CCH | N |
| 3-635 | 3,5-Cl2 | O | H | — | CH2 | CH2 | NCO2CH3 |
| 3-636 | 3,5-Cl2 | O | H | — | CH2 | CH2 | NCO2C4H9-tert |
| 3-637 | 3,5-Cl2 | O | H | — | CH2 | CH2 | N(C═O)NHCH3 |
| 3-638 | 3,5-Cl2 | O | H | — | CH2 | CH2 | N(C═O)NHC2H5 |
| 3-639 | 3,5-Cl2 | O | H | — | CH2 | CH2 | N(C═O)NHPr-n |
| 3-640 | 3,5-Cl2 | O | H | — | CH2 | CH2 | N(C═O)NHPr-i |
| 3-641 | 3,5-Cl2 | O | H | — | CH2 | CH2 | N(C═O)NHPr-c |
| 3-642 | 3,5-Cl2 | O | H | — | CH2 | CH2 | N(C═O)NHCH2Pr-c |
| 3-643 | 3,5-Cl2 | O | H | — | CH2 | CH2 | N(C═O)NHCH2CF3 |
| 3-644 | 3,5-Cl2 | O | H | — | CH2 | CH2 | N(C═O)NHCH2(py-2-yl) |
| 3-645 | 3,5-Cl2 | O | H | — | CH2 | CH2 | N(C═O)NHCH2CHCH2 |
| 3-646 | 3,5-Cl2 | O | H | — | CH2 | CH2 | N(C═O)NHCH2CCH |
| 3-647 | 3,5-Cl2 | O | H | — | CH2 | CH2 | N(C═O)NH(2-Cl—Ph) |
| 3-648 | 3,5-Cl2 | O | H | — | CH2 | CH2 | N(C═O)NH(3-Cl—Ph) |
| 3-649 | 3,5-Cl2 | O | H | — | CH2 | CH2 | N(C═O)NH(4-Cl—Ph) |
| 3-650 | 3,5-Cl2 | O | H | — | CH2 | CH2 | N(C═O)NHCH2(2-Cl—Ph) |
| 3-651 | 3,5-Cl2 | O | H | — | CH2 | CH2 | N(C═O)N(CH3)2 |
| 3-652 | 3,5-Cl2 | O | H | — | CH2 | CH2 | N(C═S)NHCH3 |
| 3-653 | 3,5-Cl2 | O | H | CH2 | CH2 | CH2 | NCO2CH3 |
| 3-654 | 3,5-Cl2 | O | H | CH2 | CH2 | CH2 | NCO2C4H9-tert |
| 3-655 | 3,5-Cl2 | O | H | CH2 | CH2 | CH2 | N(C═O)NHCH3 |
| 3-656 | 3,5-Cl2 | O | H | CH2 | CH2 | CH2 | N(C═O)NHC2H5 |
| 3-657 | 3,5-Cl2 | O | H | CH2 | CH2 | CH2 | N(C═O)NHPr-n |
| 3-658 | 3,5-Cl2 | O | H | CH2 | CH2 | CH2 | N(C═O)NHPr-i |
| 3-659 | 3,5-Cl2 | O | H | CH2 | CH2 | CH2 | N(C═O)NHPr-c |
| 3-660 | 3,5-Cl2 | O | H | CH2 | CH2 | CH2 | N(C═O)NHCH2Pr-c |
| 3-661A | 3,5-Cl2 | O | H | CH2 | CH2 | CH2 | N(C═O)NHCH2CF3 |
| 3-661B | 3,5-Cl2 | O | H | CH2 | CH2 | CH2 | N(C═O)NHBu-c |
| 3-662 | 3,5-Cl2 | O | H | CH2 | CH2 | CH2 | N(C═O)NHCH2(py-2-yl) |
| 3-663 | 3,5-Cl2 | O | H | CH2 | CH2 | CH2 | N(C═O)NHCH2CHCH2 |
| 3-664 | 3,5-Cl2 | O | H | CH2 | CH2 | CH2 | N(C═O)NHCH2CCH |
| 3-665 | 3,5-Cl2 | O | H | CH2 | CH2 | CH2 | N(C═O)NH(2-Cl—Ph) |
| 3-666 | 3,5-Cl2 | O | H | CH2 | CH2 | CH2 | N(C═O)NH(3-Cl—Ph) |
| 3-667 | 3,5-Cl2 | O | H | CH2 | CH2 | CH2 | N(C═O)NH(4-Cl—Ph) |
| 3-668 | 3,5-Cl2 | O | H | CH2 | CH2 | CH2 | N(C═O)NHCH2(2-Cl—Ph) |
| 3-669 | 3,5-Cl2 | O | H | CH2 | CH2 | CH2 | N(C═O)N(CH3)2 |
| 3-670 | 3,5-Cl2 | O | H | CH2 | CH2 | CH2 | N(C═S)NHCH3 |
| 3-671 | 3,5-(CF3)2 | O | H | — | CH2 | CH2 | NCO2CH3 |
| 3-672 | 3,5-(CF3)2 | O | H | — | CH2 | CH2 | NCO2C4H9-tert |
| 3-673 | 3,5-(CF3)2 | O | H | — | CH2 | CH2 | N(C═O)NHCH3 |
| 3-674 | 3,5-(CF3)2 | O | H | — | CH2 | CH2 | N(C═O)NHC2H5 |
| 3-675 | 3,5-(CF3)2 | O | H | — | CH2 | CH2 | N(C═O)NHPr-n |
| 3-676 | 3,5-(CF3)2 | O | H | — | CH2 | CH2 | N(C═O)NHPr-i |
| 3-677 | 3,5-(CF3)2 | O | H | — | CH2 | CH2 | N(C═O)NHPr-c |
| 3-678 | 3,5-(CF3)2 | O | H | — | CH2 | CH2 | N(C═O)NHCH2Pr-c |
| 3-679 | 3,5-(CF3)2 | O | H | — | CH2 | CH2 | N(C═O)NHCH2CF3 |
| 3-680 | 3,5-(CF3)2 | O | H | — | CH2 | CH2 | N(C═O)NHCH2(py-2-yl) |
| 3-681 | 3,5-(CF3)2 | O | H | — | CH2 | CH2 | N(C═O)NHCH2CHCH2 |
| 3-682 | 3,5-(CF3)2 | O | H | — | CH2 | CH2 | N(C═O)NHCH2CCH |
| 3-683 | 3,5-(CF3)2 | O | H | — | CH2 | CH2 | N(C═O)NH(2-Cl—Ph) |
| 3-684 | 3,5-(CF3)2 | O | H | — | CH2 | CH2 | N(C═O)NH(3-Cl—Ph) |
| 3-685 | 3,5-(CF3)2 | O | H | — | CH2 | CH2 | N(C═O)NH(4-Cl—Ph) |
| 3-686 | 3,5-(CF3)2 | O | H | — | CH2 | CH2 | N(C═O)NHCH2(2-Cl—Ph) |
| 3-687 | 3,5-(CF3)2 | O | H | — | CH2 | CH2 | N(C═O)N(CH3)2 |
| 3-688 | 3,5-(CF3)2 | O | H | — | CH2 | CH2 | N(C═S)NHCH3 |
| 3-689 | 3,4,5-Cl3 | O | H | — | CH2 | CH2 | NCO2CH3 |
| 3-690 | 3,4,5-Cl3 | O | H | — | CH2 | CH2 | NCO2C4H9-tert |
| 3-691 | 3,4,5-Cl3 | O | H | — | CH2 | CH2 | N(C═O)NHCH3 |
| 3-692 | 3,4,5-Cl3 | O | H | — | CH2 | CH2 | N(C═O)NHC2H5 |
| 3-693 | 3,4,5-Cl3 | O | H | — | CH2 | CH2 | N(C═O)NHPr-n |
| 3-694 | 3,4,5-Cl3 | O | H | — | CH2 | CH2 | N(C═O)NHPr-i |
| 3-695 | 3,4,5-Cl3 | O | H | — | CH2 | CH2 | N(C═O)NHPr-c |
| 3-696 | 3,4,5-Cl3 | O | H | — | CH2 | CH2 | N(C═O)NHCH2Pr-c |
| 3-697 | 3,4,5-Cl3 | O | H | — | CH2 | CH2 | N(C═O)NHCH2CF3 |
| 3-698 | 3,4,5-Cl3 | O | H | — | CH2 | CH2 | N(C═O)NHCH2(py-2-yl) |
| 3-699 | 3,4,5-Cl3 | O | H | — | CH2 | CH2 | N(C═O)NHCH2CHCH2 |
| 3-700 | 3,4,5-Cl3 | O | H | — | CH2 | CH2 | N(C═O)NHCH2CCH |
| 3-701 | 3,4,5-Cl3 | O | H | — | CH2 | CH2 | N(C═O)NH(2-Cl—Ph) |
| 3-702 | 3,4,5-Cl3 | O | H | — | CH2 | CH2 | N(C═O)NH(3-Cl—Ph) |
| 3-703 | 3,4,5-Cl3 | O | H | — | CH2 | CH2 | N(C═O)NH(4-Cl—Ph) |
| 3-704 | 3,4,5-Cl3 | O | H | — | CH2 | CH2 | N(C═O)NHCH2(2-Cl—Ph) |
| 3-705 | 3,4,5-Cl3 | O | H | — | CH2 | CH2 | N(C═O)N(CH3)2 |
| 3-706 | 3,4,5-Cl3 | O | H | — | CH2 | CH2 | N(C═S)NHCH3 |
| 3-707 | 3,5-Cl2 | CH2 | H | — | CH2 | CH2 | NCO2CH3 |
| 3-708 | 3,5-Cl2 | CH2 | H | — | CH2 | CH2 | NCO2C4H9-tert |
| 3-709 | 3,5-Cl2 | CH2 | H | — | CH2 | CH2 | N(C═O)NHCH3 |
| 3-710 | 3,5-Cl2 | CH2 | H | — | CH2 | CH2 | N(C═O)NHC2H5 |
| 3-711 | 3,5-Cl2 | CH2 | H | — | CH2 | CH2 | N(C═O)NHPr-n |
| 3-712 | 3,5-Cl2 | CH2 | H | — | CH2 | CH2 | N(C═O)NHPr-i |
| 3-713 | 3,5-Cl2 | CH2 | H | — | CH2 | CH2 | N(C═O)NHPr-c |
| 3-714 | 3,5-Cl2 | CH2 | H | — | CH2 | CH2 | N(C═O)NHCH2Pr-c |
| 3-715 | 3,5-Cl2 | CH2 | H | — | CH2 | CH2 | N(C═O)NHCH2CF3 |
| 3-716 | 3,5-Cl2 | CH2 | H | — | CH2 | CH2 | N(C═O)NHCH2(py-2-yl) |
| 3-717 | 3,5-Cl2 | CH2 | H | — | CH2 | CH2 | N(C═O)NHCH2CHCH2 |
| 3-718 | 3,5-Cl2 | CH2 | H | — | CH2 | CH2 | N(C═O)NHCH2CCH |
| 3-719 | 3,5-Cl2 | CH2 | H | — | CH2 | CH2 | N(C═O)NH(2-Cl—Ph) |
| 3-720 | 3,5-Cl2 | CH2 | H | — | CH2 | CH2 | N(C═O)NH(3-Cl—Ph) |
| 3-721 | 3,5-Cl2 | CH2 | H | — | CH2 | CH2 | N(C═O)NH(4-Cl—Ph) |
| 3-722 | 3,5-Cl2 | CH2 | H | — | CH2 | CH2 | N(C═O)NHCH2(2-Cl—Ph) |
| 3-723 | 3,5-Cl2 | CH2 | H | — | CH2 | CH2 | N(C═O)N(CH3)2 |
| 3-724 | 3,5-Cl2 | CH2 | H | — | CH2 | CH2 | N(C═S)NHCH3 |
| 3-725 | 3,5-(CF3)2 | CH2 | H | — | CH2 | CH2 | N(C═O)NHCH3 |
| 3-726 | 3,5-(CF3)2 | CH2 | H | — | CH2 | CH2 | N(C═O)NHC2H5 |
| 3-727 | 3,5-(CF3)2 | CH2 | H | — | CH2 | CH2 | N(C═O)NHPr-c |
| 3-728 | 3,5-(CF3)2 | CH2 | H | — | CH2 | CH2 | N(C═O)NHCH2CF3 |
| 3-729 | 3,5-(CF3)2 | CH2 | H | — | CH2 | CH2 | N(C═O)NHCH2CCH |
| 3-730 | 3,4,5-Cl3 | CH2 | H | — | CH2 | CH2 | N(C═O)NHCH3 |
| 3-731 | 3,4,5-Cl3 | CH2 | H | — | CH2 | CH2 | N(C═O)NHC2H5 |
| 3-732 | 3,4,5-Cl3 | CH2 | H | — | CH2 | CH2 | N(C═O)NHPr-c |
| 3-733 | 3,4,5-Cl3 | CH2 | H | — | CH2 | CH2 | N(C═O)NHCH2CF3 |
| 3-734 | 3,4,5-Cl3 | CH2 | H | — | CH2 | CH2 | N(C═O)NHCH2CCH |
| 3-735 | 3,5-Cl2 | CH2 | H | CH2 | CH2 | CH2 | N(C═O)NHCH3 |
| 3-736 | 3,5-Cl2 | CH2 | H | CH2 | CH2 | CH2 | N(C═O)NHC2H5 |
| 3-737 | 3,5-Cl2 | CH2 | H | CH2 | CH2 | CH2 | N(C═O)NHPr-c |
| 3-738 | 3,5-Cl2 | CH2 | H | CH2 | CH2 | CH2 | N(C═O)NHCH2CF3 |
| 3-739 | 3,5-Cl2 | CH2 | H | CH2 | CH2 | CH2 | N(C═O)NHCH2CCH |
| 3-740 | 3,5-(CF3)2 | CH2 | H | CH2 | CH2 | CH2 | N(C═O)NHCH3 |
| 3-741 | 3,5-(CF3)2 | CH2 | H | CH2 | CH2 | CH2 | N(C═O)NHC2H5 |
| 3-742 | 3,5-(CF3)2 | CH2 | H | CH2 | CH2 | CH2 | N(C═O)NHPr-c |
| 3-743 | 3,5-(CF3)2 | CH2 | H | CH2 | CH2 | CH2 | N(C═O)NHCH2CF3 |
| 3-744 | 3,5-(CF3)2 | CH2 | H | CH2 | CH2 | CH2 | N(C═O)NHCH2CCH |
| 3-745 | 3,4,5-Cl3 | CH2 | H | CH2 | CH2 | CH2 | N(C═O)NHCH3 |
| 3-746 | 3,4,5-Cl3 | CH2 | H | CH2 | CH2 | CH2 | N(C═O)NHC2H5 |
| 3-747 | 3,4,5-Cl3 | CH2 | H | CH2 | CH2 | CH2 | N(C═O)NHPr-c |
| 3-748 | 3,4,5-Cl3 | CH2 | II | CH2 | CH2 | CH2 | N(C═O)NHCH2CF3 |
| 3-749 | 3,4,5-Cl3 | CH2 | H | CH2 | CH2 | CH2 | N(C═O)NHCH2CCH |
| 3-750 | 3,4,5-Cl3 | NCH3 | H | — | CH2 | CH2 | N(C═O)NHCH3 |
| 3-751 | 3,4,5-Cl3 | NCH3 | H | — | CH2 | CH2 | N(C═O)NHC2H5 |
| 3-752 | 3,4,5-Cl3 | NCH3 | H | — | CH2 | CH2 | N(C═O)NHPr-c |
| 3-753 | 3,4,5-Cl3 | NCH3 | H | — | CH2 | CH2 | N(C═O)NHCH2CF3 |
| 3-754 | 3,4,5-Cl3 | NCH3 | H | — | CH2 | CH2 | N(C═O)NHCH2CCH |
| 3-755 | 3,5-(CF3)2 | NCH3 | H | CH2 | CH2 | CH2 | N(C═O)NHCH3 |
| 3-756 | 3,5-(CF3)2 | NCH3 | H | CH2 | CH2 | CH2 | N(C═O)NHC2H5 |
| 3-757 | 3,5-(CF3)2 | NCH3 | H | CH2 | CH2 | CH2 | N(C═O)NHPr-c |
| 3-758 | 3,5-(CF3)2 | NCH3 | H | CH2 | CH2 | CH2 | N(C═O)NHCH2CF3 |
| 3-759 | 3,5-(CF3)2 | NCH3 | H | CH2 | CH2 | CH2 | N(C═O)NHCH2CCH |
| 3-760 | 3,5-Cl2 | O | H | — | CH2 | N | NCO2CH3 |
| 3-761 | 3,5-Cl2 | O | H | — | CH2 | N | N(C═O)NHCH3 |
| 3-762 | 3,5-Cl2 | O | H | — | CH2 | N | N(C═O)NHC2H5 |
| 3-763 | 3,5-Cl2 | O | H | — | CH2 | N | N(C═O)NHPr-n |
| 3-764 | 3,5-Cl2 | O | H | — | CH2 | N | N(C═O)NHPr-i |
| 3-765 | 3,5-Cl2 | O | H | — | CH2 | N | N(C═O)NHPr-c |
| 3-766 | 3,5-Cl2 | O | H | — | CH2 | N | N(C═O)NHCH2Pr-c |
| 3-767 | 3,5-Cl2 | O | H | — | CH2 | N | N(C═O)NHCH2CF3 |
| 3-768 | 3,5-Cl2 | O | H | — | CH2 | N | N(C═O)NHCH2(py-2-yl) |
| 3-769 | 3,5-Cl2 | O | H | — | CH2 | N | N(C═O)NHCH2CHCH2 |
| 3-770 | 3,5-Cl2 | O | H | — | CH2 | N | N(C═O)NHCH2CCH |
| 3-771 | 3,5-Cl2 | O | H | — | CH2 | N | N(C═O)NH(2-Cl—Ph) |
| 3-772 | 3,5-Cl2 | O | H | — | CH2 | N | N(C═O)NH(3-Cl—Ph) |
| 3-773 | 3,5-Cl2 | O | H | — | CH2 | N | N(C═O)NH(4-Cl—Ph) |
| 3-774 | 3,5-Cl2 | O | H | — | CH2 | N | N(C═O)NHCH2(2-Cl—Ph) |
| 3-775 | 3,5-Cl2 | O | H | — | CH2 | N | N(C═O)N(CH3)2 |
| 3-776 | 3,5-Cl2 | O | H | — | CH2 | N | N(C═S)NHCH3 |
| 3-777 | 3,5-Cl2 | O | H | CH | CH | CCl | N |
| 3-778 | 3,5-Cl2 | O | H | C═O | NH | NH | CH2 |
| 3-779 | 3,5-Cl2 | O | H | CH | CH | C═O | NH |
| 3-780 | 3,5-Cl2 | O | H | — | CH2 | CH2 | C═NN═C(CH3)2 |
| 3-781 | 3,5-Cl2 | O | H | — | NH | C═O | CHSCH3 |
| 3-782 | 3,5-Cl2 | O | H | — | O | CF2 | O |
| 3-783 | 3,5-Cl2 | O | H | — | NH | C═O | CHSO2CH3 |
| 3-784 | 3,5-Cl2 | O | H | — | CH2 | C═CHN(CH3)2 | C═O |
| 3-785 | 3,4,5-Cl3 | O | H | — | CH2 | O | CHOH |
| 3-786 | 3,5-Cl2 | O | H | — | CH2 | NH | CHNH(C═O)NHC2H5 |
| 3-787 | 3,5-F2 | O | H | — | CH2 | CH2 | CHNH(C═O)NHC2H5 |
| 3-788 | 3,5-Br2 | O | H | — | CH2 | CH2 | CHNH(C═O)NHC2H5 |
| 3-789 | 3,5-I2 | O | H | — | CH2 | CH2 | CHNH(C═O)NHC2H5 |
| 3-790 | 3-F | O | H | — | CH2 | CH2 | CHNH(C═O)NHC2H5 |
| 3-791 | 3-Cl | O | H | — | CH2 | CH2 | CHNH(C═O)NHC2H5 |
| 3-792 | 3-Br | O | H | — | CH2 | CH2 | CHNH(C═O)NHC2H5 |
| 3-793 | 3-CF3 | O | H | — | CH2 | CH2 | CHNH(C═O)NHC2H5 |
| 3-794 | 4-CF3 | O | H | — | CH2 | CH2 | CHNH(C═O)NHC2H5 |
| 3-795 | 3-NO2 | O | H | — | CH2 | CH2 | CHNH(C═O)NHC2H5 |
| 3-796 | 3-CH3 | O | H | — | CH2 | CH2 | CHNH(C═O)NHC2H5 |
| 3-797 | 3-CH3O | O | H | — | CH2 | CH2 | CHNH(C═O)NHC2H5 |
| 3-798 | 3-CN | O | H | — | CH2 | CH2 | CHNH(C═O)NHC2H5 |
| 3-799 | 3-CF3O | O | H | — | CH2 | CH2 | CHNH(C═O)NHC2H5 |
| 3-800 | 3-CH3S | O | H | — | CH2 | CH2 | CHNH(C═O)NHC2H5 |
| 3-801 | 3-CH3S(O) | O | H | — | CH2 | CH2 | CHNH(C═O)NHC2H5 |
| 3-802 | 3-CH3S(O)2 | O | H | — | CH2 | CH2 | CHNH(C═O)NHC2H5 |
| 3-803 | 3-CF3S | O | H | — | CH2 | CH2 | CHNH(C═O)NHC2H5 |
| 3-804 | 3-CF3S(O) | O | H | — | CH2 | CH2 | CHNH(C═O)NHC2H5 |
| 3-805 | 3-CF3S(O)2 | O | H | — | CH2 | CH2 | CHNH(C═O)NHC2H5 |
| 3-806 | 3-OH | O | H | — | CH2 | CH2 | CHNH(C═O)NHC2H5 |
| 3-807 | 3-SH | O | H | — | CH2 | CH2 | CHNH(C═O)NHC2H5 |
| 3-808 | 3-NH2 | O | H | — | CH2 | CH2 | CHNH(C═O)NHC2H5 |
| 3-809 | 3-NHCOCH3 | O | H | — | CH2 | CH2 | CHNH(C═O)NHC2H5 |
| 3-810 | 3-NHCOCF3 | O | H | — | CH2 | CH2 | CHNH(C═O)NHC2H5 |
| 3-811 | 3-NHCO2CH3 | O | H | — | CH2 | CH2 | CHNH(C═O)NHC2H5 |
| 3-812 | 3-NHCO2CH2CCl3 | O | H | — | CH2 | CH2 | CHNH(C═O)NHC2H5 |
| 3-813 | 3-NHSO2CH3 | O | H | — | CH2 | CH2 | CHNH(C═O)NHC2H5 |
| 3-814 | 3-NHSO2CF3 | O | H | — | CH2 | CH2 | CHNH(C═O)NHC2H5 |
| 3-815 | 3-NHCH3 | O | H | — | CH2 | CH2 | CHNH(C═O)NHC2H5 |
| 3-816 | 3-N(CH3)2 | O | H | — | CH2 | CH2 | CHNH(C═O)NHC2H5 |
| 3-817 | 3-NHCOBu-t | O | H | — | CH2 | CH2 | CHNH(C═O)NHC2H5 |
| 3-818 | 3-Cl, 4-CF3 | O | H | — | CH2 | CH2 | CHNH(C═O)NHC2H5 |
| 3-819 | 3-Cl, 5-CF3 | O | H | — | CH2 | CH2 | CHNH(C═O)NHC2H5 |
| 3-820 | 3-Cl, 5-SCF3 | O | H | — | CH2 | CH2 | CHNH(C═O)NHC2H5 |
| 3-821 | 3,4,5-F3 | O | H | — | CH2 | CH2 | CHNH(C═O)NHC2H5 |
| 3-822 | 3,4,5-Br3 | O | H | — | CH2 | CH2 | CHNH(C═O)NHC2H5 |
| 3-823 | 3,5-Cl2, 4-Br | O | H | — | CH2 | CH2 | CHNH(C═O)NHC2H5 |
| 3-824 | 3,5-Br2, 4-Cl | O | H | — | CH2 | CH2 | CHNH(C═O)NHC2H5 |
| 3-825 | 3,5-Cl2, 4-NH2 | O | H | — | CH2 | CH2 | CHNH(C═O)NHC2H5 |
| 3-826 | 3,5-Br2, 4-NH2 | O | H | — | CH2 | CH2 | CHNH(C═O)NHC2H5 |
| 3-827 | 3,5-Cl2, 4-CF3 | O | H | — | CH2 | CH2 | CHNH(C═O)NHC2H5 |
| 3-828 | 3,5-Cl2 | O | H | CH2 | CH2 | CH2 | N(C═O)NHBu-c |
| 3-829 | 3,5-Cl2 | O | H | CH2 | CH2 | CH2 | CHNH(1,3-oxazolin)-2-yl |
| 3-830 | 3,5-(CF3)2 | O | H | — | CH2 | CH2 | CHO(C═O)CH3 |
| 3-831 | 3,5-(CF3)2 | O | H | — | CH2 | CH2 | CHNH2 |
| 3-832 | 3,5-(CF3)2 | O | H | — | CH2 | CH2 | CHNH(C═O)N(CH3)2 |
| 3-833 | 3,5-(CF3)2 | O | H | — | CH2 | CH2 | CHNHCO(Ph-2-Cl) |
| 3-834 | 3,5-(CF3)2 | O | H | — | CH2 | CH2 | CHNHCO(Ph-3-Cl) |
| 3-835 | 3,5-(CF3)2 | O | H | — | CH2 | CH2 | CHNH(C═O)Bu-c |
| 3-836 | 3,5-(CF3)2 | O | H | — | CH2 | CH2 | CHNH(C═O)NHCH2CF3 |
| 3-837 | 3,5-(CF3)2 | O | H | — | CH2 | CH2 | CHNH(C═O)NH2 |
| 3-838 | 3,5-(CF3)2 | O | H | — | CH2 | CH2 | CHNH(C═O)NHPr-c |
| 3-839 | 3,5-(CF3)2 | O | H | — | CH2 | CH2 | CHNH(C═O)(azetidine-1) |
| 3-840 | 3,5-(CF3)2 | CH2 | H | — | CH2 | CH2 | CHNHCO2C4H9-tert |
| 3-841 | 3,4,5-Cl3 | O | H | — | CH2 | CH2 | CHNH(C═O)NH2 |
| 3-842 | 3,4,5-Cl3 | O | H | — | CH2 | CH2 | CHNHSO2CHF2 |
| 3-843 | 3,4,5-Cl3 | O | H | — | CH2 | CH2 | CHNHSO2CH2CF3 |
| 3-844 | 3,4,5-Cl3 | O | H | — | CH2 | CH2 | CHNH(C═O)(pyrrolidine-1) |
| 3-845 | 3,4,5-Cl3 | O | H | — | CH2 | CH2 | CHNH(C═O)CH2(C═O)CH3 |
| 3-846 | 3,4,5-Cl3 | O | H | — | CH2 | CH2 | CHNH(C═O)CH2CN |
| 3-847 | 3,4,5-Cl3 | O | H | — | CH2 | CH2 | CHNH(C═O)CH═CH2 |
| 3-848 | 3,4,5-Cl3 | O | H | — | CH2 | CH2 | CHNHCO(furan-2) |
| 3-849 | 3,4,5-Cl3 | O | H | — | CH2 | CH2 | CHNH(C═O)NHCH2CN |
| 3-850 | 3,4,5-Cl3 | O | H | — | CH2 | CH2 | CHNH(C═O)CH2N(CH3)2 |
| 3-851 | 3,4,5-Cl3 | O | H | — | CH2 | CH2 | CHNH(C═O)NHCH2Pr-c |
| 3-852 | 3,4,5-Cl3 | O | H | — | CH2 | CH2 | CHNH(C═O)Pr-n |
| 3-853 | 3,4,5-Cl3 | O | H | — | CH2 | CH2 | CHNH(C═O)CH2SCH3 |
| 3-854 | 3,4,5-Cl3 | O | H | — | CH2 | CH2 | CHNH(C═O)CH2S(═O)2CH3 |
| 3-855 | 3,4,5-Cl3 | O | H | — | CH2 | CH2 | CHNHCO(2,2-F2—Pr-c) |
| 3-856 | 3,4,5-Cl3 | O | H | — | CH2 | CH2 | CHNH(C═O)NH(2-F—Pr-c) |
| 3-857 | 3,5-Cl2 | O | H | — | CH2 | CH2 | CH(1,2,3-triazole-4-TMS)-1 |
| 3-858 | 3,5-Cl2 | O | H | — | CH2 | CH2 | CH(1,2,3-triazole)-1 |
| 3-859 | 3,5-Cl2 | O | H | — | CH2 | CH2 | CHNH(C═O)NHPh |
| 3-860 | 3,5-Cl2 | O | H | — | CH2 | CH2 | CHNH(C═O)NH(Ph—Cl-2) |
| 3-861 | 3,5-Cl2 | O | H | — | CH2 | CH2 | CHNH(C═O)NH(Ph—CF3-2) |
| 3-862 | 3,5-Cl2 | O | H | — | CH2 | CH2 | CHNH(C═O)NHOCH3 |
| 3-863 | 3,5-Cl2 | O | H | — | CH2 | CH2 | CHNH(C═O)NHOC2H5 |
| 3-864 | 3,5-Cl2 | O | H | — | CH2 | CH2 | CHNH(C═O)NHOCH2CH═CH2 |
| 3-865 | 3,5-Cl2 | O | H | — | CH2 | CH2 | CHNH(C═O)NCH3OCH3 |
| 3-866 | 3,5-Cl2 | O | H | — | CH2 | CH2 | CHNH(C═O)CH(CH3)(C═O)CH3 |
| 3-867 | 3,5-Cl2 | O | H | — | CH2 | CH2 | CHNH(C═O)(N—CH3-pyrrolidine-2) |
| 3-868 | 3,5-Cl2 | O | H | — | CH2 | CH2 | CHNH(C═O)CH2Pr-c |
| 3-869 | 3,5-Cl2 | O | H | — | CH2 | CH2 | CHNH(C═O)Bu-n |
| 3-870 | 3,5-Cl2 | O | H | — | CH2 | CH2 | CHNH(C═O)NHPr-n |
| 3-871 | 3,5-Cl2 | O | H | — | CH2 | CH2 | CH(succinimide-1) |
| 3-872 | 3,5-Cl2 | O | H | — | CH2 | CH2 | CHNH(C═O)CH2SCH3 |
| 3-873 | 3,5-Cl2 | O | H | — | CH2 | CH2 | CHNH(C═O)CH2S(═O)CH3 |
| 3-874 | 3,5-Cl2 | O | H | — | CH2 | CH2 | CHNH(C═O)CH2S(═O)2CH3 |
| 3-875 | 3,5-Cl2 | O | H | — | CH2 | CH2 | CHNH(C═O)CH2SC2H5 |
| 3-876 | 3,5-Cl2 | O | H | — | CH2 | CH2 | CHNH(C═O)CH2CH═CH2 |
| 3-877 | 3,5-Cl2 | O | H | — | CH2 | CH2 | CHNH(C═O)NHCH2CCH |
| 3-878 | 3,5-Cl2 | O | H | — | CH2 | CH2 | CHNH(C═O)Pr-n |
| 3-879 | 3,5-Cl2 | O | H | — | CH2 | CH2 | CHN3 |
| 3-880 | 3,5-Cl2, 4-CF3 | O | H | — | CH2 | CH2 | CHNH(C═O)CH2CF3 |
| 3-881 | 3-CF3, 4-F | O | H | — | CH2 | CH2 | CHNH(C═O)CH2CF3 |
| 3-882 | 3-Cl | O | H | — | CH2 | CH2 | CHNH(C═O)CH2CF3 |
| 3-883 | 3,5-F2 | O | H | — | CH2 | CH2 | CHNH(C═O)CH2CF3 |
| 3-884 | 4-CF3 | O | H | — | CH2 | CH2 | CHNH(C═O)CH2CF3 |
| 3-885 | 3-NO2 | O | H | — | CH2 | CH2 | CHNH(C═O)CH2CF3 |
| 3-886 | 3-CN | O | H | — | CH2 | CH2 | CHNH(C═O)CH2CF3 |
| 3-887 | 3-CF3 | O | H | — | CH2 | CH2 | CHNH(C═O)CH2CF3 |
| 3-888 | 3-Cl, 5-CF3 | O | H | — | CH2 | CH2 | CHNH(C═O)CH2CF3 |
| 3-889 | 3-SCH3 | O | H | — | CH2 | CH2 | CHNH(C═O)CH2CF3 |
| 3-890 | 3,4-Cl2 | O | H | — | CH2 | CH2 | CHNH(C═O)CH2CF3 |
| 3-891 | 3,4,5-F3 | O | H | — | CH2 | CH2 | CHNH(C═O)CH2CF3 |
| 3-892 | 3-CF3,5-NO2 | O | H | — | CH2 | CH2 | CHNH(C═O)CH2CF3 |
| 3-893 | 3-OCF3 | O | H | — | CH2 | CH2 | CHNH(C═O)CH2CF3 |
| 3-894 | 3-OCH3 | O | H | — | CH2 | CH2 | CHNH(C═O)CH2CF3 |
| 3-895 | 4-NO2 | O | H | — | CH2 | CH2 | CHNH(C═O)C2H5 |
| 3-896 | 3-NO2, 5-CH3 | O | H | — | CH2 | CH2 | CHNH(C═O)C2H5 |
| 3-897 | 3,5-Cl2, 4-CF3 | O | H | — | CH2 | CH2 | CHNH(C═O)C2H5 |
| 3-898 | 3-CF3, 4-F | O | H | — | CH2 | CH2 | CHNH(C═O)C2H5 |
| 3-899 | 3-Cl, 4-CF3 | O | H | — | CH2 | CH2 | CHNH(C═O)C2H5 |
| 3-900 | 3,4-Cl2 | O | H | — | CH2 | CH2 | CHNH(C═O)C2H5 |
| 3-901 | 3-CN | O | H | — | CH2 | CH2 | CHNH(C═O)C2H5 |
| 3-902 | 3-NO2, 4-CH3 | O | H | — | CH2 | CH2 | CHNH(C═O)C2H5 |
| 3-903 | 4-NO2 | O | H | — | CH2 | CH2 | CHNH(C═O)C2H5 |
| 3-904 | 3,5-Cl2 | O | H | — | CH2 | CH2 | CHNH(C═O)CH2CF3 |
| 3-905 | 3,4,5-Cl3 | O | H | — | CH2 | CH2 | CHNH(C═O)CH2CF3 |
| 3-906 | 3,5-(CF3)2 | O | H | — | CH2 | CH2 | CHNH(C═O)CH2CF3 |
| 3-907 | 3,5-Cl2 | CH2 | H | — | CH2 | CH2 | CHNH(C═O)CH2CF3 |
| 3-908 | 3,4,5-Cl3 | CH2 | H | — | CH2 | CH2 | CHNH(C═O)CH2CF3 |
| 3-909 | 3,5-(CF3)2 | CH2 | H | — | CH2 | CH2 | CHNH(C═O)CH2CF3 |
| 3-910 | 3,4,5-Cl3 | CH2 | H | CH | CH | CNHCO2C4H9-tert | CH |
| 3-911 | 3,4,5-Cl3 | CH2 | H | CH | CH | CNH2 | CH |
| 3-912 | 3,4,5-Cl3 | CH2 | H | CH | CH | CNH(C═O)CH3 | CH |
| 3-913 | 3,4,5-Cl3 | CH2 | H | CH | CH | CNH(C═O)C2H5 | CH |
| 3-914 | 3,4,5-Cl3 | CH2 | H | CH | CH | CNH(C═O)Pr-c | CH |
| 3-915 | 3,4,5-Cl3 | CH2 | H | CH | CH | CNH(C═O)CH2CF3 | CH |
| 3-916 | 3,4,5-Cl3 | CH2 | H | CH | CH | CNH(C═O)NHC2H5 | CH |
| 3-917 | 3,4,5-Cl3 | O | H | CH2 | CH2 | CH2 | CHNH(C═O)CH2CN |
| 3-918 | 3,5-(CF3)2 | O | H | CH2 | CH2 | CH2 | CHNH(C═O)CH2CN |
| 3-919 | 3,5-(CF3)2 | O | H | CH2 | CH2 | CH2 | CHNH(C═O)CH2(C═O)CH3 |
| 3-920 | 3,4,5-Cl3 | CH2 | H | CH | CH | CH | CNHCO2C4H9-tert |
| 3-921 | 3,4,5-Cl3 | CH2 | H | CH | CH | CNH2 | CH |
| 3-922 | 3,4,5-Cl3 | CH2 | H | CH2 | CH2 | CH2 | CHNHCO2C4H9-tert |
| 3-923 | 3-CF3 | O | H | — | CH2 | CH2 | CHO(C═O)CH3 |
| 3-924 | 3-CF3 | O | H | — | CH2 | CH2 | CHNH2 |
| 3-925 | 3-CF3 | O | H | — | CH2 | CH2 | CHNH(C═O)CH3 |
| 3-926 | 3-CF3 | O | H | — | CH2 | CH2 | CHNH(C═O)C2H5 |
| 3-927 | 3-CF3 | O | H | — | CH2 | CH2 | CHNH(C═O)Pr-c |
| 3-928 | 3-CF3 | O | H | — | CH2 | CH2 | CHNH(C═O)CH2Pr-c |
| 3-929 | 3-CF3 | O | H | — | CH2 | CH2 | CHNH(C═O)CH2CH═CH2 |
| 3-930 | 3-CF3 | O | H | — | CH2 | CH2 | CHNH(C═O)CH2SCH3 |
| 3-931 | 3-CF3 | O | H | — | CH2 | CH2 | CHNH(C═O)CH2S(═O)CH3 |
| 3-932 | 3-CF3 | O | H | — | CH2 | CH2 | CHNH(C═O)CH2S(═O)2CH3 |
| 3-933 | 3-CF3 | O | H | — | CH2 | CH2 | CHNH(C═O)NHPr-c |
| 3-934 | 3-CF3 | O | H | — | CH2 | CH2 | CHNH(C═O)NHCH2CCH |
| 3-935 | 3-CF3 | CH2 | H | — | CH2 | CH2 | CHO(C═O)CH3 |
| 3-936 | 3-CF3 | CH2 | H | — | CH2 | CH2 | CHNH2 |
| 3-937 | 3-CF3 | CH2 | H | — | CH2 | CH2 | CHNH(C═O)CH3 |
| 3-938 | 3-CF3 | CH2 | H | — | CH2 | CH2 | CHNH(C═O)C2H5 |
| 3-939 | 3-CF3 | CH2 | H | — | CH2 | CH2 | CHNH(C═O)Pr-c |
| 3-940 | 3-CF3 | CH2 | H | — | CH2 | CH2 | CHNH(C═O)CH2Pr-c |
| 3-941 | 3-CF3 | CH2 | H | — | CH2 | CH2 | CHNH(C═O)CH2CH═CH2 |
| 3-942 | 3-CF3 | CH2 | H | — | CH2 | CH2 | CHNH(C═O)CH2SCH3 |
| 3-943 | 3-CF3 | CH2 | H | — | CH2 | CH2 | CHNH(C═O)CH2S(═O)CH3 |
| 3-944 | 3-CF3 | CH2 | H | — | CH2 | CH2 | CHNH(C═O)CH2S(═O)2CH3 |
| 3-945 | 3-CF3 | CH2 | H | — | CH2 | CH2 | CHNH(C═O)NHPr-c |
| 3-946 | 3-CF3 | CH2 | H | — | CH2 | CH2 | CHNH(C═O)NHCH2CCH |

TABLE 4

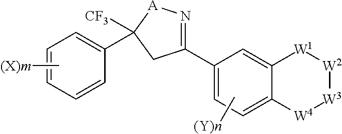

| Example | (X)m | A | (Y)n | W¹ | W² | W³ | W⁴ |
|---|---|---|---|---|---|---|---|
| 4-1 | 3,5-Cl2 | O | H | — | O | N | CNH2 |
| 4-2 | 3,5-Cl2 | O | H | — | O | N | CNH(C═O)CH3 |
| 4-3 | 3,5-Cl2 | O | H | — | O | N | CNH(C═O)C2H5 |
| 4-4 | 3,5-Cl2 | O | H | — | O | N | CNH(C═O)CH2CF3 |
| 4-5 | 3,5-Cl2 | O | H | — | O | N | CNH(C═O)Pr-c |
| 4-6 | 3,5-Cl2 | O | H | — | O | N | CNH(C═O)Pr-i |
| 4-7 | 3,5-Cl2 | O | H | — | O | N | CNH(C═O)CH2Pr-c |
| 4-8 | 3,5-Cl2 | O | H | — | O | N | CNH(C═O)CH2SMe |
| 4-9 | 3,5-Cl2 | O | H | — | O | N | CNH(C═O)CH2SOMe |
| 4-10 | 3,5-Cl2 | O | H | — | O | N | CNH(C═O)CH2SO2Me |
| 4-11 | 3,5-Cl2 | O | H | — | O | N | CNH(C═O)CF3 |
| 4-12 | 3,5-Cl2 | O | H | — | O | N | CNH(C═O)NHCH3 |
| 4-13 | 3,5-Cl2 | O | H | — | O | N | CNH(C═O)NHC2H5 |
| 4-14 | 3,5-Cl2 | O | H | — | O | N | CNH(C═O)NHCH2CCH |
| 4-15 | 3,5-Cl2 | O | H | — | O | N | CNH(C═S)C2H5 |
| 4-16 | 3,5-Cl2 | O | H | — | O | N | CNH(C═S)NHCH3 |
| 4-17 | 3,5-Cl2 | O | H | — | O | N | CNH(C═O)OCH3 |
| 4-18 | 3,5-Cl2 | O | H | — | O | N | CNH(C═O)OC4H9-tert |
| 4-19 | 3,5-Cl2 | O | H | — | O | N | CN((C═O)Pr-i)2 |
| 4-20 | 3,4,5-Cl3 | O | H | — | O | N | CNH2 |
| 4-21 | 3,4,5-Cl3 | O | H | — | O | N | CNH(C═O)CH3 |
| 4-22 | 3,4,5-Cl3 | O | H | — | O | N | CNH(C═O)C2H5 |
| 4-23 | 3,4,5-Cl3 | O | H | — | O | N | CNH(C═O)CH2CF3 |
| 4-24 | 3,4,5-Cl3 | O | H | — | O | N | CNH(C═O)Pr-c |
| 4-25 | 3,4,5-Cl3 | O | H | — | O | N | CNH(C═O)CH2Pr-c |
| 4-26 | 3,4,5-Cl3 | O | H | — | O | N | CNH(C═O)CH2SMe |
| 4-27 | 3,4,5-Cl3 | O | H | — | O | N | CNH(C═O)CH2SOMe |
| 4-28 | 3,4,5-Cl3 | O | H | — | O | N | CNH(C═O)CH2SO2Me |
| 4-29 | 3,4,5-Cl3 | O | H | — | O | N | CNH(C═O)NHCH3 |
| 4-30 | 3,4,5-Cl3 | O | H | — | O | N | CNH(C═O)NHC2H5 |
| 4-31 | 3,4,5-Cl3 | O | H | — | O | N | CNH(C═O)NHCH2CCH |
| 4-32 | 3,4,5-Cl3 | O | H | — | O | N | CNH(C═S)C2H5 |
| 4-33 | 3,4,5-Cl3 | O | H | — | O | N | CNH(C═S)NHCH3 |
| 4-34 | 3,4,5-Cl3 | O | H | — | O | N | CNH(C═O)OC4H9-tert |
| 4-35 | 3,4,5-Cl3 | O | H | — | O | N | CHNH(C═O)NHBu-t |
| 4-36 | 3,4,5-Cl3 | O | H | — | O | N | CHNH(C═O)NHBu-c |
| 4-37 | 3,5-Br2 | O | H | — | O | N | CNH(C═O)C2H5 |
| 4-38 | 3,4-Cl2, 5-CF3 | O | H | — | O | N | CNH(C═O)CH2CF3 |
| 4-39 | 3,5-(CF3)2 | O | H | — | O | N | CNH(C═O)C2H5 |
| 4-40 | 3-CF3 | O | H | — | O | N | CNH(C═O)CH2CF3 |
| 4-41 | 3,5-Cl2 | O | H | CNH2 | N | O | — |
| 4-42 | 3,5-Cl2 | O | H | CNH(C═O)C2H5 | N | O | — |
| 4-43 | 3,5-Cl2 | O | H | CNH(C═O)Pr-i | N | O | — |
| 4-44 | 3,5-Cl2 | O | H | CNH(C═O)CF3 | N | O | — |
| 4-45 | 3,5-Cl2 | O | H | CNH(C═O)CH2CF3 | N | O | — |
| 4-46 | 3,5-Cl2 | O | H | CNH(C═O)NHC2H5 | N | O | — |
| 4-47 | 3,5-Cl2 | O | H | CNH(C═O)NHPr-i | N | O | — |
| 4-48 | 3,5-Cl2 | O | H | CNH(C═O)OCH3 | N | O | — |
| 4-49 | 3,5-Cl2 | O | H | CNH(C═O)OPh | N | O | — |
| 4-50 | 3,5-Cl2 | O | H | — | NCH3 | N | CNH2 |
| 4-51 | 3,5-Cl2 | O | H | — | NCH3 | N | CNH(C═O)C2H5 |
| 4-52 | 3,5-Cl2 | O | H | — | NCH3 | N | CNH(C═O)CH2CF3 |
| 4-53 | 3,5-Cl2 | O | H | — | NCH3 | N | CNH(C═O)NHC2H5 |
| 4-54 | 3,5-Cl2 | O | H | CNH2 | N | NCH3 | — |
| 4-55 | 3,5-Cl2 | O | H | CNH(C═O)C2H5 | N | NCH3 | — |
| 4-56 | 3,5-Cl2 | O | H | CNH(C═O)Pr-i | N | NCH3 | — |
| 4-57 | 3,5-Cl2 | O | H | CNH(C═O)CF3 | N | NCH3 | — |
| 4-58 | 3,5-Cl2 | O | H | CNH(C═O)CH2CF3 | N | NCH3 | — |
| 4-59 | 3,5-Cl2 | O | H | CNH(C═O)NHC2H5 | N | NCH3 | — |
| 4-60 | 3,5-Cl2 | O | H | CNH(C═O)NHPr-i | N | NCH3 | — |
| 4-61 | 3,5-Cl2 | O | H | CNH(C═O)OCH3 | N | NCH3 | — |
| 4-62 | 3,5-Cl2 | O | H | CNH(C═O)OPh | N | NCH3 | — |
| 4-63 | 3,5-Cl2 | O | H | — | SO2 | N | CNH2 |
| 4-64 | 3,5-Cl2 | O | H | — | SO2 | N | CNH(C═O)C2H5 |
| 4-65 | 3,5-Cl2 | O | H | — | SO2 | N | CNH(C═O)CH2CF3 |
| 4-66 | 3,5-Cl2 | O | H | — | SO2 | N | CNH(C═O)NHC2H5 |
| 4-67 | 3,5-Cl2 | O | H | CNH2 | N | SO2 | — |
| 4-68 | 3,5-Cl2 | O | H | CNH(C═O)C2H5 | N | SO2 | — |
| 4-69 | 3,5-Cl2 | O | H | CNH(C═O)CH2CF3 | N | SO2 | — |
| 4-70 | 3,5-Cl2 | O | H | CNH(C═O)NHC2H5 | N | SO2 | — |
| 4-71 | 3,4,5-Cl3 | O | H | — | NCH3 | N | CNH2 |
| 4-72 | 3,4,5-Cl3 | O | H | — | NCH3 | N | CNH(C═O)C2H5 |
| 4-73 | 3,4,5-Cl3 | O | H | — | NCH3 | N | CNH(C═O)CH2CF3 |
| 4-74 | 3,4,5-Cl3 | O | H | — | NCH3 | N | CNH(C═O)NHC2H5 |
| 4-75 | 3,5-Br2 | O | H | — | NCH3 | N | CNH(C═O)C2H5 |
| 4-76 | 3,4-Cl2, 5-CF3 | O | H | — | NCH3 | N | CNH(C═O)C2H5 |
| 4-77 | 3,5-(CF3)2 | O | H | — | NCH3 | N | CNH(C═O)C2H5 |
| 4-78 | 3-CF3 | O | H | — | NCH3 | N | CNH(C═O)C2H5 |
| 4-79 | 3,4,5-Cl3 | O | H | — | SO2 | N | CNH(C═O)C2H5 |
| 4-80 | 3,5-(CF3)2 | O | H | — | SO2 | N | CNH(C═O)C2H5 |
| 4-81 | 3,4-Cl2, 5-CF3 | O | H | — | SO2 | N | CNH(C═O)C2H5 |
| 4-82 | 3,5-Cl2 | O | H | — | CH2 | N | CNH2 |
| 4-83 | 3,5-Cl2 | O | H | — | CH2 | N | CNH(C═O)CH3 |
| 4-84 | 3,5-Cl2 | O | H | — | CH2 | N | CNH(C═O)C2H5 |
| 4-85 | 3,5-Cl2 | O | H | — | CH2 | N | CNH(C═O)CH2CF3 |
| 4-86 | 3,5-Cl2 | O | H | — | CH2 | N | CNH(C═O)Pr-c |
| 4-87 | 3,5-Cl2 | O | H | — | CH2 | N | CNH(C═O)CH2Pr-c |
| 4-88 | 3,5-Cl2 | O | H | — | CH2 | N | CNH(C═O)CH2SMe |
| 4-89 | 3,5-Cl2 | O | H | — | CH2 | N | CNH(C═O)CH2SOMe |
| 4-90 | 3,5-Cl2 | O | H | — | CH2 | N | CNH(C═O)CH2SO2Me |
| 4-91 | 3,5-Cl2 | O | H | — | CH2 | N | CNH(C═O)NHCH2CCH |
| 4-92 | 3,5-Cl2 | O | H | — | CH2 | N | CNH(C═O)OC4H9-tert |
| 4-93 | 3,4,5-Cl3 | O | H | — | CH2 | N | CNH(C═O)C2H5 |
| 4-94 | 3,4,5-Cl3 | O | H | — | CH2 | N | CNH(C═O)CH2CF3 |
| 4-95 | 3,4,5-Cl3 | O | H | — | CH2 | N | CNH(C═O)Pr-c |
| 4-96 | 3,4,5-Cl3 | O | H | — | CH2 | N | CNH(C═O)CH2SO2Me |
| 4-97 | 3,5-Br2 | O | H | — | CH2 | N | CNH(C═O)CH2CF3 |
| 4-98 | 3,4-Cl2, 5-CF3 | O | H | — | CH2 | N | CNH(C═O)CH2CF3 |
| 4-99 | 3,5-(CF3)2 | O | H | — | CH2 | N | CNH(C═O)CH2CF3 |
| 4-100 | 3-CF3 | O | H | — | CH2 | N | CNH(C═O)CH2CF3 |
| 4-101 | 3,5-Cl2 | O | H | — | O | CH2 | CHNH2 |
| 4-102 | 3,5-Cl2 | O | H | — | O | CH2 | CHNH(C═O)CH3 |
| 4-103 | 3,5-Cl2 | O | H | — | O | CH2 | CHNH(C═O)C2H5 |
| 4-104 | 3,5-Cl2 | O | H | — | O | CH2 | CHNH(C═O)CH2CF3 |
| 4-105 | 3,5-Cl2 | O | H | — | O | CH2 | CHNH(C═O)Pr-c |
| 4-106 | 3,5-Cl2 | O | H | — | O | CH2 | CHNH(C═O)CH2Pr-c |
| 4-107 | 3,5-Cl2 | O | H | — | O | CH2 | CHNH(C═O)CH2SMe |
| 4-108 | 3,5-Cl2 | O | H | — | O | CH2 | CHNH(C═O)CH2SOMe |
| 4-109 | 3,5-Cl2 | O | H | — | O | CH2 | CHNH(C═O)CH2SO2Me |
| 4-110 | 3,5-Cl2 | O | H | — | O | CH2 | CHNH(C═O)NHC2H5 |
| 4-111 | 3,5-Cl2 | O | H | — | O | CH2 | CHNH(C═O)NHCH2CCH |
| 4-112 | 3,5-Cl2 | O | H | — | O | CH2 | CHNH(C═O)OC4H9-tert |
| 4-113 | 3,4,5-Cl3 | O | H | — | O | CH2 | CHNH(C═O)C2H5 |
| 4-114 | 3,4,5-Cl3 | O | H | — | O | CH2 | CHO(C═O)CH3 |
| 4-115 | 3,4,5-Cl3 | O | H | — | O | CH2 | CHNH(C═O)CH2CF3 |
| 4-116 | 3,4,5-Cl3 | O | H | — | O | CH2 | CHNH(C═O)Pr-c |
| 4-117 | 3,4,5-Cl3 | O | H | — | O | CH2 | CHNH(C═O)CH2SO2Me |
| 4-118 | 3,5-Br2 | O | H | — | O | CH2 | CHNH(C═O)CH2CF3 |
| 4-119 | 3,4-Cl2, 5-CF3 | O | H | — | O | CH2 | CHNH(C═O)CH2CF3 |
| 4-120 | 3,5-(CF3)2 | O | H | — | O | CH2 | CHNH(C═O)CH2CF3 |
| 4-121 | 3-CF3 | O | H | — | O | CH2 | CHNH(C═O)CH2CF3 |
| 4-122 | 3,4,5-Cl3 | CH2 | H | — | O | CH2 | CHNH2 |
| 4-123 | 3,4,5-Cl3 | CH2 | H | — | O | CH2 | CHNH(C═O)C2H5 |
| 4-124 | 3,4,5-Cl3 | CH2 | H | — | O | CH2 | CHNH(C═O)CH2CF3 |
| 4-125 | 3,4,5-Cl3 | CH2 | H | — | O | CH2 | CHNH(C═O)Pr-c |
| 4-126 | 3,4,5-Cl3 | CH2 | H | — | O | CH2 | CHNH(C═O)NHC2H5 |
| 4-127 | 3,4,5-Cl3 | CH2 | H | — | O | CH2 | CHNH(C═O)—OC4H9-tert |
| 4-128 | 3,4,5-Cl3 | O | H | — | S | CH2 | CHNH2 |
| 4-129 | 3,4,5-Cl3 | O | H | — | S | CH2 | CHNH(C═O)CH3 |
| 4-130 | 3,4,5-Cl3 | O | H | — | S | CH2 | CHNH(C═O)C2H5 |
| 4-131 | 3,4,5-Cl3 | O | H | — | S | CH2 | CHNH(C═O)CH2CF3 |
| 4-132 | 3,4,5-Cl3 | O | H | — | S | CH2 | CHNH(C═O)Pr-c |
| 4-133 | 3,4,5-Cl3 | O | H | — | S | CH2 | CHNH(C═O)CH2Pr-c |
| 4-134 | 3,4,5-Cl3 | O | H | — | S | CH2 | CHNH(C═O)—CH2SMe |
| 4-135 | 3,4,5-Cl3 | O | H | — | S | CH2 | CHNH(C═O)—CH2SOMe |
| 4-136 | 3,4,5-Cl3 | O | H | — | S | CH2 | CHNH(C═O)—CH2SO2Me |
| 4-137 | 3,4,5-Cl3 | O | H | — | S | CH2 | CHNH(C═O)—NHC2H5 |
| 4-138 | 3,4,5-Cl3 | O | H | — | S | CH2 | CHNH(C═O)—NHCH2CCH |
| 4-139 | 3,4,5-Cl3 | O | H | — | S | CH2 | CHNH(C═O)—OC4H9-tert |
| 4-140 | 3,5-Cl2 | O | H | — | S | CH2 | CHNH(C═O)C2H5 |
| 4-141 | 3,5-Cl2 | O | H | — | S | CH2 | CHO(C═O)CH3 |
| 4-142 | 3,5-Cl2 | O | H | — | S | CH2 | CHNH(C═O)CH2CF3 |
| 4-143 | 3,5-Cl2 | O | H | — | S | CH2 | CHNH(C═O)Pr-c |
| 4-144 | 3,5-Cl2 | O | H | — | S | CH2 | CHNH(C═O)CH2SO2Me |
| 4-145 | 3,5-Br2 | O | H | — | S | CH2 | CHNH(C═O)CH2CF3 |
| 4-146 | 3,4-Cl2, 5-CF3 | O | H | — | S | CH2 | CHNH(C═O)CH2CF3 |
| 4-147 | 3,5-(CF3)2 | O | H | — | S | CH2 | CHNH(C═O)CH2CF3 |
| 4-148 | 3-CF3 | O | H | — | S | CH2 | CHNH(C═O)CH2CF3 |
| 4-149 | 3,4,5-Cl3 | CH2 | H | — | S | CH2 | CHNH2 |
| 4-150 | 3,4,5-Cl3 | CH2 | H | — | S | CH2 | CHNH(C═O)C2H5 |
| 4-151 | 3,4,5-Cl3 | CH2 | H | — | S | CH2 | CHNH(C═O)CH2CF3 |
| 4-152 | 3,4,5-Cl3 | CH2 | H | — | S | CH2 | CHNH(C═O)Pr-c |
| 4-153 | 3,4,5-Cl3 | CH2 | H | — | S | CH2 | CHNH(C═O)—NHC2H5 |
| 4-154 | 3,4,5-Cl3 | CH2 | H | — | S | CH2 | CHNH(C═O)—OC4H9-tert |
| 4-155 | 3,5-Cl2 | O | H | — | NCH3 | CH2 | CHNH2 |
| 4-156 | 3,5-Cl2 | O | H | — | NCH3 | CH2 | CHNH(C═O)CH3 |
| 4-157 | 3,5-Cl2 | O | H | — | NCH3 | CH2 | CHNH(C═O)C2H5 |
| 4-158 | 3,5-Cl2 | O | H | — | NCH3 | CH2 | CHNH(C═O)CH2CF3 |
| 4-159 | 3,5-Cl2 | O | H | — | NCH3 | CH2 | CHNH(C═O)Pr-c |
| 4-160 | 3,5-Cl2 | O | H | — | NCH3 | CH2 | CHNH(C═O)CH2Pr-c |
| 4-161 | 3,5-Cl2 | O | H | — | NCH3 | CH2 | CHNH(C═O)CH2—SMe |
| 4-162 | 3,5-Cl2 | O | H | — | NCH3 | CH2 | CHNH(C═O)CH2—SOMe |
| 4-163 | 3,5-Cl2 | O | H | — | NCH3 | CH2 | CHNH(C═O)CH2—SO2Me |
| 4-164 | 3,5-Cl2 | O | H | — | NCH3 | CH2 | CHNH(C═O)NH—C2H5 |
| 4-165 | 3,5-Cl2 | O | H | — | NCH3 | CH2 | CHNH(C═O)NH—CH2CCH |
| 4-166 | 3,5-Cl2 | O | H | — | NCH3 | CH2 | CHNH(C═O)O—C4H9-tert |
| 4-167 | 3,4,5-Cl3 | O | H | — | NCH3 | CH2 | CHNH(C═O)C2H5 |
| 4-168 | 3,4,5-Cl3 | O | H | — | NCH3 | CH2 | CHO(C═O)CH3 |
| 4-169 | 3,4,5-Cl3 | O | H | — | NCH3 | CH2 | CHNH(C═O)CH2CF3 |
| 4-170 | 3,4,5-Cl3 | O | H | — | NCH3 | CH2 | CHNH(C═O)Pr-c |
| 4-171 | 3,4,5-Cl3 | O | H | — | NCH3 | CH2 | CHNH(C═O)—CH2SO2Me |
| 4-172 | 3,5-Br2 | O | H | — | NCH3 | CH2 | CHNH(C═O)CH2CF3 |
| 4-173 | 3,4-Cl2, 5-CF3 | O | H | — | NCH3 | CH2 | CHNH(C═O)CH2CF3 |
| 4-174 | 3,5-(CF3)2 | O | H | — | NCH3 | CH2 | CHNH(C═O)—CH2CF3 |
| 4-175 | 3-CF3 | O | H | — | NCH3 | CH2 | CHNH(C═O)—CH2CF3 |
| 4-176 | 3,4,5-Cl3 | CH2 | H | — | NCH3 | CH2 | CHNH2 |
| 4-177 | 3,4,5-Cl3 | CH2 | H | — | NCH3 | CH2 | CHNH(C═O)C2H5 |
| 4-178 | 3,4,5-Cl3 | CH2 | H | — | NCH3 | CH2 | CHNH(C═O)CH2CF3 |
| 4-179 | 3,4,5-Cl3 | CH2 | H | — | NCH3 | CH2 | CHNH(C═O)Pr-c |
| 4-180 | 3,4,5-Cl3 | CH2 | H | — | NCH3 | CH2 | CHNH(C═O)NH—C2H5 |
| 4-181 | 3,4,5-Cl3 | CH2 | H | — | NCH3 | CH2 | CHNH(C═O)O—C4H9-tert |
| 4-182 | 3,4,5-Cl3 | O | H | — | CH2 | O | C═NH(C═O)—C2H5 |
| 4-183 | 3,4,5-Cl3 | O | H | — | CH2 | O | C═NHCOCH2CF3 |
| 4-184 | 3,4,5-Cl3 | O | H | — | CH2 | O | C═NHC(C═O)—NHC2H5 |
| 4-185 | 3,4,5-Cl3 | O | H | — | CH2 | NCH2(py-2-yl) | C═NCOC2H5 |
| 4-186 | 3,4,5-Cl3 | O | H | — | CH2 | NCH2(py-2-yl) | C═NHCOCH2CF3 |
| 4-187 | 3,4,5-Cl3 | O | H | — | CH2 | NCH2(py-2-yl) | C═NCOPr-c |
| 4-188 | 3,4,5-Cl3 | O | H | — | CH2 | NCH2(py-2-yl) | C═NHC(C═O)—NHC2H5 |
| 4-189 | 3,4,5-Cl3 | O | H | — | CH2 | NCH3 | C═NCOC2H5 |
| 4-190 | 3,4,5-Cl3 | O | H | — | CH2 | NCH3 | C═NHCOCH2CF3 |
| 4-191 | 3,4,5-Cl3 | O | H | — | CH2 | NCH3 | C═NCOPr-c |
| 4-192 | 3,4,5-Cl3 | O | H | — | CH2 | NCH3 | C═NHC(C═O)—NHC2H5 |
| 4-193 | 3,4,5-Cl3 | O | H | — | CH2 | NCH2CF3 | C═NCOC2H5 |
| 4-194 | 3,4,5-Cl3 | O | H | — | CH2 | NCH2CF3 | C═NHCOCH2CF3 |
| 4-195 | 3,4,5-Cl3 | O | H | — | CH2 | NCH2CF3 | C═NCOPr-c |
| 4-196 | 3,4,5-Cl3 | O | H | — | CH2 | NCH2CF3 | C═NHC(C═O)—NHC2H5 |
| 4-197 | 3,5-Cl2 | O | H | — | CH | N | CHNH2 |
| 4-198 | 3,5-Cl2 | O | H | — | CH | N | CHNH(C═O)C2H5 |
| 4-199 | 3,5-(CF3)2 | O | H | — | CH | N | CHNH(C═O)CH2CF3 |
| 4-200 | 3,5-(CF3)2 | O | H | — | CH | N | CHNH(C═O)Pr-c |
| 4-201 | 3,5-(CF3)2 | O | H | — | CH | N | CHNH(C═O)—NHC2H5 |
| 4-202 | 3,4,5-Cl3 | O | H | — | CH | N | CHNH(C═O)—OC4H9-tert |
| 4-203 | 3,5-Cl2 | O | H | CH | CH | N | CNH(C═O)—OC4H9-tert |
| 4-204 | 3,4,5-Cl3 | O | H | CH | CH | N | CNH(C═O)C2H5 |
| 4-205 | 3,4,5-Cl3 | O | H | CH | CH | N | CNH(C═O)CH2CF3 |
| 4-206 | 3,4,5-Cl3 | O | H | CH | CH | N | CNH(C═O)Pr-c |
| 4-207 | 3,4,5-Cl3 | O | H | CH | CH | N | CNH(C═O)—CH2SO2Me |
| 4-208 | 3,4,5-Cl3 | O | H | CH | CH | N | CNH(C═O)CH2CF3 |
| 4-209 | 3,5-Br2 | O | H | CH | CH | N | CNH(C═O)CH2CF3 |
| 4-210 | 3,4-Cl2, 5-CF3 | O | H | CH | CH | N | CNH(C═O)CH2CF3 |
| 4-211 | 3,5-(CF3)2 | O | H | CH | CH | N | CNH(C═O)CH2CF3 |
| 4-212 | 3-CF3 | O | H | CH | CH | N | CNH(C═O)CH2CF3 |
| 4-213 | 3,4,5-Cl3 | CH2 | H | CH | CH | N | CNH2 |
| 4-214 | 3,4,5-Cl3 | CH2 | H | CH | CH | N | CNH(C═O)C2H5 |
| 4-215 | 3,4,5-Cl3 | CH2 | H | CH | CH | N | CNH(C═O)CH2CF3 |
| 4-216 | 3,4,5-Cl3 | CH2 | H | CH | CH | N | CNH(C═O)Pr-c |
| 4-217 | 3,4,5-Cl3 | CH2 | H | CH | CH | N | CNH(C═O)NHC2H5 |
| 4-218 | 3,4,5-Cl3 | CH2 | H | CH | CH | N | CNH(C═O)—OC4H9-tert |
| 4-219 | 3,4,5-Cl3 | O | H | CH | N | CH | CNH2 |
| 4-220 | 3,4,5-Cl3 | O | H | CH | N | CH | CNH(C═O)C2H5 |
| 4-221 | 3,4,5-Cl3 | O | H | CH | N | CH | CNH(C═O)CH2CF3 |
| 4-222 | 3,4,5-Cl3 | O | H | CH | N | CH | CNH(C═O)Pr-c |
| 4-223 | 3,4,5-Cl3 | O | H | CH | N | CH | CNH(C═O)NHC2H5 |
| 4-224 | 3,4,5-Cl3 | O | H | CH | N | CH | CNH(C═O)O—C4H9-tert |
| 4-225 | 3,5-(CF3)2 | CH2 | H | N | CH | N | CNH2 |
| 4-226 | 3,5-(CF3)2 | CH2 | H | N | CH | N | CNH(C═O)C2H5 |
| 4-227 | 3,5-(CF3)2 | CH2 | H | N | CH | N | CNH(C═O)CH2CF3 |
| 4-228 | 3,5-(CF3)2 | CH2 | H | N | CH | N | CNH(C═O)Pr-c |
| 4-229 | 3,5-(CF3)2 | CH2 | H | N | CH | N | CNH(C═O)NHC2H5 |
| 4-230 | 3,5-(CF3)2 | CH2 | H | N | CH | N | CNH(C═O)O—C4H9-tert |
| 4-231 | 3,5-Cl2 | O | H | CH | N | N | CNH2 |
| 4-232 | 3,5-Cl2 | O | H | CH | N | N | CNH(C═O)CH3 |
| 4-233 | 3,5-Cl2 | O | H | CH | N | N | CNH(C═O)C2H5 |
| 4-234 | 3,5-Cl2 | O | H | CH | N | N | CNH(C═O)CH2CF3 |
| 4-235 | 3,5-Cl2 | O | H | CH | N | N | CNH(C═O)Pr-c |
| 4-236 | 3,5-Cl2 | O | H | CH | N | N | CNH(C═O)CH2Pr-c |
| 4-237 | 3,5-Cl2 | O | H | CH | N | N | CNH(C═O)CH2SMe |
| 4-238 | 3,5-Cl2 | O | H | CH | N | N | CNH(C═O)CH2—SOMe |
| 4-239 | 3,5-Cl2 | O | H | CH | N | N | CNH(C═O)—CH2SO2Me |
| 4-240 | 3,5-Cl2 | O | H | CH | N | N | CNH(C═O)NH—CH2CCH |
| 4-241 | 3,5-Cl2 | O | H | CH | N | N | CNH(C═O)NH—C2H5 |
| 4-242 | 3,5-Cl2 | O | H | CH | N | N | CNH(C═O)O—C4H9-tert |
| 4-243 | 3,4,5-Cl3 | O | H | CH | N | N | CNH(C═O)C2H5 |
| 4-244 | 3,4,5-Cl3 | O | H | CH | N | N | CNH(C═O)CH2CF3 |
| 4-245 | 3,4,5-Cl3 | O | H | CH | N | N | CNH(C═O)Pr-c |
| 4-246 | 3,4,5-Cl3 | O | H | CH | N | N | CNH(C═O)—CH2SO2Me |
| 4-247 | 3,4,5-Cl3 | O | H | CH | N | N | CNH(C═O)CH2CF3 |
| 4-248 | 3,5-Br2 | O | H | CH | N | N | CNH(C═O)CH2CF3 |
| 4-249 | 3,4-Cl2, 5-CF3 | O | H | CH | N | N | CNH(C═O)CH2CF3 |
| 4-250 | 3,5-(CF3)2 | O | H | CH | N | N | CNH(C═O)CH2CF3 |
| 4-251 | 3-CF3 | O | H | CH | N | N | CNH(C═O)CH2CF3 |
| 4-252 | 3,4,5-Cl3 | CH2 | H | CH | N | N | CNH2 |
| 4-253 | 3,4,5-Cl3 | CH2 | H | CH | N | N | CNH(C═O)C2H5 |
| 4-254 | 3,4,5-Cl3 | CH2 | H | CH | N | N | CNH(C═O)CH2CF3 |
| 4-255 | 3,4,5-Cl3 | CH2 | H | CH | N | N | CNH(C═O)Pr-c |
| 4-256 | 3,4,5-Cl3 | CH2 | H | CH | N | N | CNH(C═O)NHC2H5 |
| 4-257 | 3,4,5-Cl3 | CH2 | H | CH | N | N | CNH(C═O)O—C4H9-tert |
| 4-258 | 3,5-(CF3)2 | O | H | N | CH | N | CNH2 |
| 4-259 | 3,5-(CF3)2 | O | H | N | CH | N | CNH(C═O)C2H5 |
| 4-260 | 3,5-(CF3)2 | O | H | N | CH | N | CNH(C═O)CH2CF3 |
| 4-261 | 3,5-(CF3)2 | O | H | N | CH | N | CNH(C═O)Pr-c |
| 4-262 | 3,5-(CF3)2 | O | H | N | CH | N | CNH(C═O)NHC2H5 |
| 4-263 | 3,5-(CF3)2 | O | H | N | CH | N | CNH(C═O)O—C4H9-tert |
| 4-264 | 3,5-Cl2 | CH2 | H | N | N | CH | CNH2 |
| 4-265 | 3,5-Cl2 | CH2 | H | N | N | CH | CNH(C═O)C2H5 |
| 4-266 | 3,5-Cl2 | CH2 | H | N | N | CH | CNH(C═O)CH2CF3 |
| 4-267 | 3,5-Cl2 | CH2 | H | N | N | CH | CNH(C═O)Pr-c |
| 4-268 | 3,5-Cl2 | CH2 | H | N | N | CH | CNH(C═O)NHC2H5 |
| 4-269 | 3,5-Cl2 | CH2 | H | N | N | CH | CNH(C═O)O—C4H9-tert |
| 4-270 | 3,4,5-Cl3 | O | H | N | N | N | CNH2 |
| 4-271 | 3,4,5-Cl3 | O | H | N | N | N | CNH(C═O)C2H5 |
| 4-272 | 3,4,5-Cl3 | O | H | N | N | N | CNH(C═O)CH2CF3 |
| 4-273 | 3,4,5-Cl3 | O | H | N | N | N | CNH(C═O)Pr-c |
| 4-274 | 3,4,5-Cl3 | O | H | N | N | N | CNH(C═O)NH—C2H5 |
| 4-275 | 3,4,5-Cl3 | O | H | N | N | N | CNH(C═O)O—C4H9-tert |
| 4-276 | 3,4,5-Cl3 | CH2 | H | N | N | N | CNH2 |
| 4-277 | 3,4,5-Cl3 | CH2 | H | N | N | N | CNH(C═O)C2H5 |
| 4-278 | 3,4,5-Cl3 | CH2 | H | N | N | N | CNH(C═O)CH2CF3 |
| 4-279 | 3,4,5-Cl3 | CH2 | H | N | N | N | CNH(C═O)Pr-c |
| 4-280 | 3,4,5-Cl3 | CH2 | H | N | N | N | CNH(C═O)NHC2H5 |
| 4-281 | 3,4,5-Cl3 | CH2 | H | N | N | N | CNH(C═O)O—C4H9-tert |
| 4-282 | 3,4,5-Cl3 | O | H | CH2 | NCH3 | N | CNH2 |
| 4-283 | 3,4,5-Cl3 | O | H | CH2 | NCH3 | N | CNH(C═O)C2H5 |
| 4-284 | 3,4,5-Cl3 | O | H | CH2 | NCH3 | N | CNH(C═O)CH2CF3 |
| 4-285 | 3,4,5-Cl3 | O | H | CH2 | NCH3 | N | CNH(C═O)Pr-c |
| 4-286 | 3,4,5-Cl3 | O | H | CH2 | NCH3 | N | CNH(C═O)NHC2H5 |
| 4-287 | 3,4,5-Cl3 | O | H | CH2 | NCH3 | N | CNH(C═O)O—C4H9-tert |
| 4-288 | 3,4,5-Cl3 | O | H | CH2 | O | N | CNH2 |
| 4-289 | 3,4,5-Cl3 | O | H | CH2 | O | N | CNH(C═O)C2H5 |
| 4-290 | 3,4,5-Cl3 | O | H | CH2 | O | N | CNH(C═O)CH2CF3 |
| 4-291 | 3,4,5-Cl3 | O | H | CH2 | O | N | CNH(C═O)Pr-c |
| 4-292 | 3,4,5-Cl3 | O | H | CH2 | O | N | CNH(C═O)NHC2H5 |
| 4-293 | 3,4,5-Cl3 | O | H | CH2 | O | N | CNH(C═O)O—C4H9-tert |
| 4-294 | 3,4,5-Cl3 | S | H | CH2 | O | N | CNH2 |
| 4-295 | 3,4,5-Cl3 | S | H | CH2 | O | N | CNH(C═O)C2H5 |
| 4-296 | 3,4,5-Cl3 | S | H | CH2 | O | N | CNH(C═O)CH2CF3 |
| 4-297 | 3,4,5-Cl3 | S | H | CH2 | O | N | CNH(C═O)Pr-c |
| 4-298 | 3,4,5-Cl3 | S | H | CH2 | O | N | CNH(C═O)NHC2H5 |
| 4-299 | 3,4,5-Cl3 | S | H | CH2 | O | N | CNH(C═O)O—C4H9-tert |
| 4-300 | 3,4,5-Cl3 | S | H | — | O | N | CNH2 |
| 4-301 | 3,4,5-Cl3 | S | H | — | O | N | CNH(C═O)C2H5 |
| 4-302 | 3,4,5-Cl3 | S | H | — | O | N | CNH(C═O)CH2CF3 |
| 4-303 | 3,4,5-Cl3 | S | H | — | O | N | CNH(C═O)Pr-c |
| 4-304 | 3,4,5-Cl3 | S | H | — | O | N | CNH(C═O)NHC2H5 |
| 4-305 | 3,4,5-Cl3 | S | H | — | O | N | CNH(C═O)O—C4H9-tert |

TABLE 5

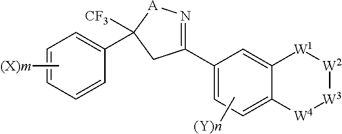

| Example | (X)m | A | (Y)n | W¹ | W² | W³ | W⁴ |
|---|---|---|---|---|---|---|---|
| 5-1 | 3,5-Cl2 | O | H | — | CH2 | CHCH3 | CHNH2 |
| 5-2 | 3,5-Cl2 | O | H | — | CH2 | CHCH3 | CHNH(C═O)CH3 |
| 5-3 | 3,5-Cl2 | O | H | — | CH2 | CHCH3 | CHNH(C═O)C2H5 |
| 5-4 | 3,5-Cl2 | O | H | — | CH2 | CHCH3 | CHNH(C═O)CH2CF3 |
| 5-5 | 3,5-Cl2 | O | H | — | CH2 | CHCH3 | CHNH(C═O)Pr-c |
| 5-6 | 3,5-Cl2 | O | H | — | CH2 | CHCH3 | CHNH(C═O)Pr-i |
| 5-7 | 3,5-Cl2 | O | H | — | CH2 | CHCH3 | CHNH(C═O)CH2Pr-c |
| 5-8 | 3,5-Cl2 | O | H | — | CH2 | CHCH3 | CHNH(C═O)CH2SMe |
| 5-9 | 3,5-Cl2 | O | H | — | CH2 | CHCH3 | CHNH(C═O)CH2S(═O)Me |
| 5-10 | 3,5-Cl2 | O | H | — | CH2 | CHCH3 | CHNH(C═O)CH2S(═O)2Me |
| 5-11 | 3,5-Cl2 | O | H | — | CH2 | CHCH3 | CHNH(C═O)CF3 |
| 5-12 | 3,5-Cl2 | O | H | — | CH2 | CHCH3 | CHNH(C═O)NHCH3 |
| 5-13 | 3,5-Cl2 | O | H | — | CH2 | CHCH3 | CHNH(C═O)NHC2H5 |
| 5-14 | 3,5-Cl2 | O | H | — | CH2 | CHCH3 | CHNH(C═O)NHCH2CCH |
| 5-15 | 3,5-Cl2 | O | H | — | CH2 | CHCH3 | CHNH(C═S)C2H5 |
| 5-16 | 3,5-Cl2 | O | H | — | CH2 | CHCH3 | CHNH(C═S)NHCH3 |
| 5-17 | 3,5-Cl2 | O | H | — | CH2 | CHCH3 | CHNH(C═O)OCH3 |
| 5-18 | 3,5-Cl2 | O | H | — | CH2 | CHCH3 | CHNH(C═O)OC4H9-tert |
| 5-19 | 3,5-Cl2 | O | H | — | CH2 | CHCH3 | CHN((C═O)Pr-i)2 |
| 5-20 | 3,4,5-Cl3 | O | H | — | CH2 | CHCH3 | CHNH2 |
| 5-21 | 3,4,5-Cl3 | O | H | — | CH2 | CHCH3 | CHNH(C═O)C2H5 |
| 5-22 | 3,4,5-Cl3 | O | H | — | CH2 | CHCH3 | CHNH(C═O)CH2CF3 |
| 5-23 | 3,4,5-Cl3 | O | H | — | CH2 | CHCH3 | CHNH(C═O)Pr-c |
| 5-24 | 3,4,5-Cl3 | O | H | — | CH2 | CHCH3 | CHNH(C═O)CH2S(═O)2Me |
| 5-25 | 3,4,5-Cl3 | O | H | — | CH2 | CHCH3 | CHNH(C═O)NHC2H5 |
| 5-26 | 3,4,5-Cl3 | O | H | — | CH2 | CHCH3 | CHNH(C═O)OC4H9-tert |
| 5-27 | 3,4,5-Cl3 | CH2 | H | — | CH2 | CHCH3 | CHNH2 |
| 5-28 | 3,4,5-Cl3 | CH2 | H | — | CH2 | CHCH3 | CHNH(C═O)C2H5 |
| 5-29 | 3,4,5-Cl3 | CH2 | H | — | CH2 | CHCH3 | CHNH(C═O)CH2CF3 |
| 5-30 | 3,4,5-Cl3 | CH2 | H | — | CH2 | CHCH3 | CHNH(C═O)Pr-c |
| 5-31 | 3,4,5-Cl3 | CH2 | H | — | CH2 | CHCH3 | CHNH(C═O)CH2S(═O)2Me |
| 5-32 | 3,4,5-Cl3 | CH2 | H | — | CH2 | CHCH3 | CHNH(C═O)NHC2H5 |
| 5-33 | 3,4,5-Cl3 | CH2 | H | — | CH2 | CHCH3 | CHNH(C═O)OC4H9-tert |
| 5-34 | 3,5-(CF3)2 | O | H | — | CH2 | C(CH3)2 | CHNH2 |
| 5-35 | 3,5-(CF3)2 | O | H | — | CH2 | C(CH3)2 | CHNH(C═O)C2H5 |
| 5-36 | 3,5-(CF3)2 | O | H | — | CH2 | C(CH3)2 | CHNH(C═O)CH2CF3 |
| 5-37 | 3,5-(CF3)2 | O | H | — | CH2 | C(CH3)2 | CHNH(C═O)Pr-c |
| 5-38 | 3,5-(CF3)2 | O | H | — | CH2 | C(CH3)2 | CHNH(C═O)CH2S(═O)2Me |
| 5-39 | 3,5-(CF3)2 | O | H | — | CH2 | C(CH3)2 | CHNH(C═O)NHC2H5 |
| 5-40 | 3,5-(CF3)2 | O | H | — | CH2 | C(CH3)2 | CHNH(C═O)OC4H9-tert |
| 5-41 | 3,4-Cl, 5-CF3 | CH2 | H | — | CH2 | C(CH3)2 | CHNH2 |
| 5-42 | 3,4-Cl, 5-CF3 | CH2 | H | — | CH2 | C(CH3)2 | CHNH(C═O)C2H5 |
| 5-43 | 3,4-Cl, 5-CF3 | CH2 | H | — | CH2 | C(CH3)2 | CHNH(C═O)CH2CF3 |
| 5-44 | 3,4-Cl, 5-CF3 | CH2 | H | — | CH2 | C(CH3)2 | CHNH(C═O)Pr-c |
| 5-45 | 3,4-Cl, 5-CF3 | CH2 | H | — | CH2 | C(CH3)2 | CHNH(C═O)CH2S(═O)2Me |
| 5-46 | 3,4-Cl, 5-CF3 | CH2 | H | — | CH2 | C(CH3)2 | CHNH(C═O)NHC2H5 |
| 5-47 | 3,4-Cl, 5-CF3 | CH2 | H | — | CH2 | C(CH3)2 | CHNH(C═O)OC4H9-tert |
| 5-48 | 3,4,5-Cl3 | O | H | — | CH2 | CHF | CHNH2 |
| 5-49 | 3,4,5-Cl3 | O | H | — | CH2 | CHF | CHNH(C═O)C2H5 |
| 5-50 | 3,4,5-Cl3 | O | H | — | CH2 | CHF | CHNH(C═O)CH2CF3 |
| 5-51 | 3,4,5-Cl3 | O | H | — | CH2 | CHF | CHNH(C═O)Pr-c |
| 5-52 | 3,4,5-Cl3 | O | H | — | CH2 | CHF | CHNH(C═O)CH2S(═O)2Me |
| 5-53 | 3,4,5-Cl3 | O | H | — | CH2 | CHF | CHNH(C═O)NHC2H5 |
| 5-54 | 3,4,5-Cl3 | O | H | — | CH2 | CHF | CHNH(C═O)OC4H9-tert |
| 5-55 | 3,4,5-Cl3 | CH2 | H | — | CH2 | CHF | CHNH2 |
| 5-56 | 3,4,5-Cl3 | CH2 | H | — | CH2 | CHF | CHNH(C═O)C2H5 |
| 5-57 | 3,4,5-Cl3 | CH2 | H | — | CH2 | CHF | CHNH(C═O)CH2CF3 |
| 5-58 | 3,4,5-Cl3 | CH2 | H | — | CH2 | CHF | CHNH(C═O)Pr-c |
| 5-59 | 3,4,5-Cl3 | CH2 | H | — | CH2 | CHF | CHNH(C═O)CH2S(═O)2Me |
| 5-60 | 3,4,5-Cl3 | CH2 | H | — | CH2 | CHF | CHNH(C═O)NHC2H5 |
| 5-61 | 3,4,5-Cl3 | CH2 | H | — | CH2 | CHF | CHNH(C═O)OC4H9-tert |
| 5-62 | 3,4,5-Cl3 | O | H | — | CH2 | CHCl | CHNH2 |
| 5-63 | 3,4,5-Cl3 | O | H | — | CH2 | CHCl | CHNH(C═O)C2H5 |
| 5-64 | 3,4,5-Cl3 | O | H | — | CH2 | CHCl | CHNH(C═O)CH2CF3 |
| 5-65 | 3,4,5-Cl3 | O | H | — | CH2 | CHCl | CHNH(C═O)Pr-c |
| 5-66 | 3,4,5-Cl3 | O | H | — | CH2 | CHCl | CHNH(C═O)CH2S(═O)2Me |
| 5-67 | 3,4,5-Cl3 | O | H | — | CH2 | CHCl | CHNH(C═O)NHC2H5 |
| 5-68 | 3,4,5-Cl3 | O | H | — | CH2 | CHCl | CHNH(C═O)OC4H9-tert |
| 5-69 | 3,4,5-Cl3 | CH2 | H | — | CH2 | CHCl | CHNH2 |
| 5-70 | 3,4,5-Cl3 | CH2 | H | — | CH2 | CHCl | CHNH(C═O)C2H5 |
| 5-71 | 3,4,5-Cl3 | CH2 | H | — | CH2 | CHCl | CHNH(C═O)CH2CF3 |
| 5-72 | 3,4,5-Cl3 | CH2 | H | — | CH2 | CHCl | CHNH(C═O)Pr-c |
| 5-73 | 3,4,5-Cl3 | CH2 | H | — | CH2 | CHCl | CHNH(C═O)CH2S(═O)2Me |
| 5-74 | 3,4,5-Cl3 | CH2 | H | — | CH2 | CHCl | CHNH(C═O)NHC2H5 |
| 5-75 | 3,4,5-Cl3 | CH2 | H | — | CH2 | CHCl | CHNH(C═O)OC4H9-tert |
| 5-76 | 3,5-Cl2 | O | H | — | CH2 | CHCF3 | CHNH2 |
| 5-77 | 3,5-Cl2 | O | H | — | CH2 | CHCF3 | CHNH(C═O)C2H5 |
| 5-78 | 3,5-Cl2 | O | H | — | CH2 | CHCF3 | CHNH(C═O)CH2CF3 |
| 5-79 | 3,5-Cl2 | O | H | — | CH2 | CHCF3 | CHNH(C═O)Pr-c |
| 5-80 | 3,5-Cl2 | O | H | — | CH2 | CHCF3 | CHNH(C═O)CH2S(═O)2Me |
| 5-81 | 3,5-Cl2 | O | H | — | CH2 | CHCF3 | CHNH(C═O)NHC2H5 |
| 5-82 | 3,5-Cl2 | O | H | — | CH2 | CHCF3 | CHNH(C═O)OC4H9-tert |
| 5-83 | 3,4,5-Cl3 | CH2 | H | — | CH2 | CHSCH3 | CHNH2 |
| 5-84 | 3,4,5-Cl3 | CH2 | H | — | CH2 | CHSCH3 | CHNH(C═O)C2H5 |
| 5-85 | 3,4,5-Cl3 | CH2 | H | — | CH2 | CHSCH3 | CHNH(C═O)CH2CF3 |
| 5-86 | 3,4,5-Cl3 | CH2 | H | — | CH2 | CHSCH3 | CHNH(C═O)Pr-c |
| 5-87 | 3,4,5-Cl3 | CH2 | H | — | CH2 | CHSCH3 | CHNH(C═O)CH2S(═O)2Me |
| 5-88 | 3,4,5-Cl3 | CH2 | H | — | CH2 | CHSCH3 | CHNH(C═O)NHC2H5 |
| 5-89 | 3,4,5-Cl3 | CH2 | H | — | CH2 | CHSCH3 | CHNH(C═O)OC4H9-tert |
| 5-90 | 3,5-(CF3)2 | O | H | — | CH2 | CHS(═O)CH3 | CHNH2 |
| 5-91 | 3,5-(CF3)2 | O | H | — | CH2 | CHS(═O)CH3 | CHNH(C═O)C2H5 |
| 5-92 | 3,5-(CF3)2 | O | H | — | CH2 | CHS(═O)CH3 | CHNH(C═O)CH2CF3 |
| 5-93 | 3,5-(CF3)2 | O | H | — | CH2 | CHS(═O)CH3 | CHNH(C═O)Pr-c |
| 5-94 | 3,5-(CF3)2 | O | H | — | CH2 | CHS(═O)CH3 | CHNH(C═O)CH2S(═O)2Me |
| 5-95 | 3,5-(CF3)2 | O | H | — | CH2 | CHS(═O)CH3 | CHNH(C═O)NHC2H5 |
| 5-96 | 3,5-(CF3)2 | O | H | — | CH2 | CHS(═O)CH3 | CHNH(C═O)OC4H9-tert |
| 5-97 | 3,4-Cl2, 5-CF3 | O | H | — | CH2 | CHS(═O)2CH3 | CHNH2 |
| 5-98 | 3,4-Cl2, 5-CF3 | O | H | — | CH2 | CHS(═O)2CH3 | CHNH(C═O)C2H5 |
| 5-99 | 3,4-Cl2, 5-CF3 | O | H | — | CH2 | CHS(═O)2CH3 | CHNH(C═O)CH2CF3 |
| 5-100 | 3,4-Cl2, 5-CF3 | O | H | — | CH2 | CHS(═O)2CH3 | CHNH(C═O)Pr-c |
| 5-101 | 3,4-Cl2, 5-CF3 | O | H | — | CH2 | CHS(═O)2CH3 | CHNH(C═O)CH2S(═O)2Me |
| 5-102 | 3,4-Cl2, 5-CF3 | O | H | — | CH2 | CHS(═O)2CH3 | CHNH(C═O)NHC2H5 |
| 5-103 | 3,4-Cl2, 5-CF3 | O | H | — | CH2 | CHS(═O)2CH3 | CHNH(C═O)OC4H9-tert |
| 5-104 | 3,4,5-Cl3 | CH2 | H | — | CH2 | CHCN | CHNH2 |
| 5-105 | 3,4,5-Cl3 | CH2 | H | — | CH2 | CHCN | CHNH(C═O)C2H5 |
| 5-106 | 3,4,5-Cl3 | CH2 | H | — | CH2 | CHCN | CHNH(C═O)CH2CF3 |
| 5-107 | 3,4,5-Cl3 | CH2 | H | — | CH2 | CHCN | CHNH(C═O)Pr-c |
| 5-108 | 3,4,5-Cl3 | CH2 | H | — | CH2 | CHCN | CHNH(C═O)CH2S(═O)2Me |
| 5-109 | 3,4,5-Cl3 | CH2 | H | — | CH2 | CHCN | CHNH(C═O)NHC2H5 |
| 5-110 | 3,4,5-Cl3 | CH2 | H | — | CH2 | CHCN | CHNH(C═O)OC4H9-tert |
| 5-111 | 3,5-Cl2 | O | H | CH2 | CH2 | CHCH3 | CHNH2 |
| 5-112 | 3,5-Cl2 | O | H | CH2 | CH2 | CHCH3 | CHNH(C═O)CH3 |
| 5-113 | 3,5-Cl2 | O | H | CH2 | CH2 | CHCH3 | CHNH(C═O)C2H5 |
| 5-114 | 3,5-Cl2 | O | H | CH2 | CH2 | CHCH3 | CHNH(C═O)CH2CF3 |
| 5-115 | 3,5-Cl2 | O | H | CH2 | CH2 | CHCH3 | CHNH(C═O)Pr-c |
| 5-116 | 3,5-Cl2 | O | H | CH2 | CH2 | CHCH3 | CHNH(C═O)Pr-i |
| 5-117 | 3,5-Cl2 | O | H | CH2 | CH2 | CHCH3 | CHNH(C═O)CH2Pr-e |
| 5-118 | 3,5-Cl2 | O | H | CH2 | CH2 | CHCH3 | CHNH(C═O)CH2SMe |
| 5-119 | 3,5-Cl2 | O | H | CH2 | CH2 | CHCH3 | CHNH(C═O)CH2S(═O)Me |
| 5-120 | 3,5-Cl2 | O | H | CH2 | CH2 | CHCH3 | CHNH(C═O)CH2S(═O)2Me |
| 5-121 | 3,5-Cl2 | O | H | CH2 | CH2 | CHCH3 | CHNH(C═O)CF3 |
| 5-122 | 3,5-Cl2 | O | H | CH2 | CH2 | CHCH3 | CHNH(C═O)NHCH3 |
| 5-123 | 3,5-Cl2 | O | H | CH2 | CH2 | CHCH3 | CHNH(C═O)NHC2H5 |
| 5-124 | 3,5-Cl2 | O | H | CH2 | CH2 | CHCH3 | CHNH(C═O)NHCH2CCH |
| 5-125 | 3,5-Cl2 | O | H | CH2 | CH2 | CHCH3 | CHNH(C═S)C2H5 |
| 5-126 | 3,5-Cl2 | O | H | CH2 | CH2 | CHCH3 | CHNH(C═S)NHCH3 |
| 5-127 | 3,5-Cl2 | O | H | CH2 | CH2 | CHCH3 | CHNH(C═O)OCH3 |
| 5-128 | 3,5-Cl2 | O | H | CH2 | CH2 | CHCH3 | CHNH(C═O)OC4H9-tert |
| 5-129 | 3,5-Cl2 | O | H | CH2 | CH2 | CHCH3 | CHN((C═O)Pr-i)2 |
| 5-130 | 3,4,5-Cl3 | O | H | CH2 | CH2 | CHCH3 | CHNH2 |
| 5-131 | 3,4,5-Cl3 | O | H | CH2 | CH2 | CHCH3 | CHNH(C═O)C2H5 |
| 5-132 | 3,4,5-Cl3 | O | H | CH2 | CH2 | CHCH3 | CHNH(C═O)CH2CF3 |
| 5-133 | 3,4,5-Cl3 | O | H | CH2 | CH2 | CHCH3 | CHNH(C═O)Pr-c |
| 5-134 | 3,4,5-Cl3 | O | H | CH2 | CH2 | CHCH3 | CHNH(C═O)CH2S(═O)2Me |
| 5-135 | 3,4,5-Cl3 | O | H | CH2 | CH2 | CHCH3 | CHNH(C═O)NHC2H5 |
| 5-136 | 3,4,5-Cl3 | O | H | CH2 | CH2 | CHCH3 | CHNH(C═O)OC4H9-tert |
| 5-137 | 3,4,5-Cl3 | CH2 | H | CH2 | CH2 | CHCH3 | CHNH2 |
| 5-138 | 3,4,5-Cl3 | CH2 | H | CH2 | CH2 | CHCH3 | CHNH(C═O)C2H5 |
| 5-139 | 3,4,5-Cl3 | CH2 | H | CH2 | CH2 | CHCH3 | CHNH(C═O)CH2CF3 |
| 5-140 | 3,4,5-Cl3 | CH2 | H | CH2 | CH2 | CHCH3 | CHNH(C═O)Pr-c |
| 5-141 | 3,4,5-Cl3 | CH2 | H | CH2 | CH2 | CHCH3 | CHNH(C═O)CH2S(═O)2Me |
| 5-142 | 3,4,5-Cl3 | CH2 | H | CH2 | CH2 | CHCH3 | CHNH(C═O)NHC2H5 |
| 5-143 | 3,4,5-Cl3 | CH2 | H | CH2 | CH2 | CHCH3 | CHNH(C═O)OC4H9-tert |
| 5-144 | 3,5-(CF3)2 | O | H | CH2 | CH2 | C(CH3)2 | CHNH2 |
| 5-145 | 3,5-(CF3)2 | O | H | CH2 | CH2 | C(CH3)2 | CHNH(C═O)C2H5 |
| 5-146 | 3,5-(CF3)2 | O | H | CH2 | CH2 | C(CH3)2 | CHNH(C═O)CH2CF3 |
| 5-147 | 3,5-(CF3)2 | O | H | CH2 | CH2 | C(CH3)2 | CHNH(C═O)Pr-c |
| 5-148 | 3,5-(CF3)2 | O | H | CH2 | CH2 | C(CH3)2 | CHNH(C═O)CH2S(═O)2Me |
| 5-149 | 3,5-(CF3)2 | O | H | CH2 | CH2 | C(CH3)2 | CHNH(C═O)NHC2H5 |
| 5-150 | 3,5-(CF3)2 | O | H | CH2 | CH2 | C(CH3)2 | CHNH(C═O)OC4H9-tert |
| 5-151 | 3,4-Cl, 5-CF3 | CH2 | H | CH2 | CH2 | C(CH3)2 | CHNH2 |
| 5-152 | 3,4-Cl, 5-CF3 | CH2 | H | CH2 | CH2 | C(CH3)2 | CHNH(C═O)C2H5 |
| 5-153 | 3,4-Cl, 5-CF3 | CH2 | H | CH2 | CH2 | C(CH3)2 | CHNH(C═O)CH2CF3 |
| 5-154 | 3,4-Cl, 5-CF3 | CH2 | H | CH2 | CH2 | C(CH3)2 | CHNH(C═O)Pr-c |
| 5-155 | 3,4-Cl, 5-CF3 | CH2 | H | CH2 | CH2 | C(CH3)2 | CHNH(C═O)CH2S(═O)2Me |
| 5-156 | 3,4-Cl, 5-CF3 | CH2 | H | CH2 | CH2 | C(CH3)2 | CHNH(C═O)NHC2H5 |
| 5-157 | 3,4-Cl, 5-CF3 | CH2 | H | CH2 | CH2 | C(CH3)2 | CHNH(C═O)OC4H9-tert |
| 5-158 | 3,4,5-Cl3 | O | H | CH2 | CH2 | CHF | CHNH2 |
| 5-159 | 3,4,5-Cl3 | O | H | CH2 | CH2 | CHF | CHNH(C═O)C2H5 |
| 5-160 | 3,4,5-Cl3 | O | H | CH2 | CH2 | CHF | CHNH(C═O)CH2CF3 |
| 5-161 | 3,4,5-Cl3 | O | H | CH2 | CH2 | CHF | CHNH(C═O)Pr-c |
| 5-162 | 3,4,5-Cl3 | O | H | CH2 | CH2 | CHF | CHNH(C═O)CH2S(═O)2Me |
| 5-163 | 3,4,5-Cl3 | O | H | CH2 | CH2 | CHF | CHNH(C═O)NHC2H5 |
| 5-164 | 3,4,5-Cl3 | O | H | CH2 | CH2 | CHF | CHNH(C═O)OC4H9-tert |
| 5-165 | 3,4,5-Cl3 | CH2 | H | CH2 | CH2 | CHF | CHNH2 |
| 5-166 | 3,4,5-Cl3 | CH2 | H | CH2 | CH2 | CHF | CHNH(C═O)C2H5 |
| 5-167 | 3,4,5-Cl3 | CH2 | H | CH2 | CH2 | CHF | CHNH(C═O)CH2CF3 |
| 5-168 | 3,4,5-Cl3 | CH2 | H | CH2 | CH2 | CHF | CHNH(C═O)Pr-c |
| 5-169 | 3,4,5-Cl3 | CH2 | H | CH2 | CH2 | CHF | CHNH(C═O)CH2S(═O)2Me |
| 5-170 | 3,4,5-Cl3 | CH2 | H | CH2 | CH2 | CHF | CHNH(C═O)NHC2H5 |
| 5-171 | 3,4,5-Cl3 | CH2 | H | CH2 | CH2 | CHF | CHNH(C═O)OC4H9-tert |
| 5-172 | 3,4,5-Cl3 | O | H | CH2 | CH2 | CHCl | CHNH2 |
| 5-173 | 3,4,5-Cl3 | O | H | CH2 | CH2 | CHCl | CHNH(C═O)C2H5 |
| 5-174 | 3,4,5-Cl3 | O | H | CH2 | CH2 | CHCl | CHNH(C═O)CH2CF3 |
| 5-175 | 3,4,5-Cl3 | O | H | CH2 | CH2 | CHCl | CHNH(C═O)Pr-c |
| 5-176 | 3,4,5-Cl3 | O | H | CH2 | CH2 | CHCl | CHNH(C═O)CH2S(═O)2Me |
| 5-177 | 3,4,5-Cl3 | O | H | CH2 | CH2 | CHCl | CHNH(C═O)NHC2H5 |
| 5-178 | 3,4,5-Cl3 | O | H | CH2 | CH2 | CHCl | CHNH(C═O)OC4H9-tert |
| 5-179 | 3,4,5-Cl3 | CH2 | H | CH2 | CH2 | CHCl | CHNH2 |
| 5-180 | 3,4,5-Cl3 | CH2 | H | CH2 | CH2 | CHCl | CHNH(C═O)C2H5 |
| 5-181 | 3,4,5-Cl3 | CH2 | H | CH2 | CH2 | CHCl | CHNH(C═O)CH2CF3 |
| 5-182 | 3,4,5-Cl3 | CH2 | H | CH2 | CH2 | CHCl | CHNH(C═O)Pr-c |
| 5-183 | 3,4,5-Cl3 | CH2 | H | CH2 | CH2 | CHCl | CHNH(C═O)CH2S(═O)2Me |
| 5-184 | 3,4,5-Cl3 | CH2 | H | CH2 | CH2 | CHCl | CHNH(C═O)NHC2H5 |
| 5-185 | 3,4,5-Cl3 | CH2 | H | CH2 | CH2 | CHCl | CHNH(C═O)OC4H9-tert |
| 5-186 | 3,5-Cl2 | O | H | CH2 | CH2 | CHCF3 | CHNH2 |
| 5-187 | 3,5-Cl2 | O | H | CH2 | CH2 | CHCF3 | CHNH(C═O)C2H5 |
| 5-188 | 3,5-Cl2 | O | H | CH2 | CH2 | CHCF3 | CHNH(C═O)CH2CF3 |
| 5-189 | 3,5-Cl2 | O | H | CH2 | CH2 | CHCF3 | CHNH(C═O)Pr-c |
| 5-190 | 3,5-Cl2 | O | H | CH2 | CH2 | CHCF3 | CHNH(C═O)CH2S(═O)2Me |
| 5-191 | 3,5-Cl2 | O | H | CH2 | CH2 | CHCF3 | CHNH(C═O)NHC2H5 |
| 5-192 | 3,5-Cl2 | O | H | CH2 | CH2 | CHCF3 | CHNH(C═O)OC4H9-tert |
| 5-193 | 3,4,5-Cl3 | CH2 | H | CH2 | CH2 | CHSCH3 | CHNH2 |
| 5-194 | 3,4,5-Cl3 | CH2 | H | CH2 | CH2 | CHSCH3 | CHNH(C═O)C2H5 |
| 5-195 | 3,4,5-Cl3 | CH2 | H | CH2 | CH2 | CHSCH3 | CHNH(C═O)CH2CF3 |
| 5-196 | 3,4,5-Cl3 | CH2 | H | CH2 | CH2 | CHSCH3 | CHNH(C═O)Pr-c |
| 5-197 | 3,4,5-Cl3 | CH2 | H | CH2 | CH2 | CHSCH3 | CHNH(C═O)CH2S(═O)2Me |
| 5-198 | 3,4,5-Cl3 | CH2 | H | CH2 | CH2 | CHSCH3 | CHNH(C═O)NHC2H5 |
| 5-199 | 3,4,5-Cl3 | CH2 | H | CH2 | CH2 | CHSCH3 | CHNH(C═O)OC4H9-tert |
| 5-200 | 3,5-(CF3)2 | O | H | CH2 | CH2 | CHS(═O)CH3 | CHNH2 |
| 5-201 | 3,5-(CF3)2 | O | H | CH2 | CH2 | CHS(═O)CH3 | CHNH(C═O)C2H5 |
| 5-202 | 3,5-(CF3)2 | O | H | CH2 | CH2 | CHS(═O)CH3 | CHNH(C═O)CH2CF3 |
| 5-203 | 3,5-(CF3)2 | O | H | CH2 | CH2 | CHS(═O)CH3 | CHNH(C═O)Pr-c |
| 5-204 | 3,5-(CF3)2 | O | H | CH2 | CH2 | CHS(═O)CH3 | CHNH(C═O)CH2S(═O)2Me |
| 5-205 | 3,5-(CF3)2 | O | H | CH2 | CH2 | CHS(═O)CH3 | CHNH(C═O)NHC2H5 |
| 5-206 | 3,5-(CF3)2 | O | H | CH2 | CH2 | CHS(═O)CH3 | CHNH(C═O)OC4H9-tert |
| 5-207 | 3,4-Cl2, 5-CF3 | O | H | CH2 | CH2 | CHS(═O)2CH3 | CHNH2 |
| 5-208 | 3,4-Cl2, 5-CF3 | O | H | CH2 | CH2 | CHS(═O)2CH3 | CHNH(C═O)C2H5 |
| 5-209 | 3,4-Cl2, 5-CF3 | O | H | CH2 | CH2 | CHS(═O)2CH3 | CHNH(C═O)CH2CF3 |
| 5-210 | 3,4-Cl2, 5-CF3 | O | H | CH2 | CH2 | CHS(═O)2CH3 | CHNH(C═O)Pr-c |
| 5-211 | 3,4-Cl2, 5-CF3 | O | H | CH2 | CH2 | CHS(═O)2CH3 | CHNH(C═O)CH2S(═O)2Me |
| 5-212 | 3,4-Cl2, 5-CF3 | O | H | CH2 | CH2 | CHS(═O)2CH3 | CHNH(C═O)NHC2H5 |
| 5-213 | 3,4-Cl2, 5-CF3 | O | H | CH2 | CH2 | CHS(═O)2CH3 | CHNH(C═O)OC4H9-tert |
| 5-214 | 3,4,5-Cl3 | CH2 | H | CH2 | CH2 | CHCN | CHNH2 |
| 5-215 | 3,4,5-Cl3 | CH2 | H | CH2 | CH2 | CHCN | CHNH(C═O)C2H5 |
| 5-216 | 3,4,5-Cl3 | CH2 | H | CH2 | CH2 | CHCN | CHNH(C═O)CH2CF3 |
| 5-217 | 3,4,5-Cl3 | CH2 | H | CH2 | CH2 | CHCN | CHNH(C═O)Pr-c |
| 5-218 | 3,4,5-Cl3 | CH2 | H | CH2 | CH2 | CHCN | CHNH(C═O)CH2S(═O)2Me |
| 5-219 | 3,4,5-Cl3 | CH2 | H | CH2 | CH2 | CHCN | CHNH(C═O)NHC2H5 |
| 5-220 | 3,4,5-Cl3 | CH2 | H | CH2 | CH2 | CHCN | CHNH(C═O)OC4H9-tert |

TABLE 6

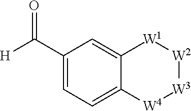

| Example | W¹ | W² | W³ | W⁴ |
|---|---|---|---|---|
| 6-1 | — | CH2 | O | C═O |
| 6-2 | — | CH2 | O | CH2 |
| 6-3 | — | CH2 | CH2 | O |
| 6-4 | — | O | CH2 | O |
| 6-5 | — | O | CF2 | O |
| 6-6 | — | CH | CH | O |
| 6-7 | — | CH2 | CH2 | S |
| 6-8 | — | CH2 | S | CH2 |
| 6-9 | — | CH | CH | S |
| 6-10 | — | CH2 | CH2 | NH |
| 6-11 | — | CH2 | NH | CH2 |
| 6-12 | — | NH | CH2 | CH2 |
| 6-13 | — | CH | CH | NH |
| 6-14 | — | CH | N | NH |
| 6-15 | — | C═O | O | CH2 |
| 6-16 | — | CH2 | S | C═O |
| 6-17 | — | N | CH | O |
| 6-18 | — | N | CH | S |
| 6-19 | — | N | CH | NH |
| 6-20 | — | O | CH | N |
| 6-21 | — | S | CH | N |
| 6-22 | — | CH | N | O |
| 6-23 | — | CH | N | S |
| 6-24 | — | CH2 | CH2 | C═O |
| 6-25 | — | CH | CH | C═O |
| 6-26 | — | CH2 | C═O | CH2 |
| 6-27 | — | C═O | CH2 | CH2 |
| 6-28 | — | CH2 | C═O | NH |
| 6-29 | — | NH | C═O | CH2 |
| 6-30 | — | CH2 | C═O | N(C═O)CH3 |
| 6-31 | — | CH2 | C═O | N(C═O)CH2CF3 |
| 6-32 | — | CH2 | CH2 | N(C═O)CH3 |
| 6-33 | — | CH2 | CH2 | N(C═O)C2H5 |
| 6-34 | — | CH2 | CH2 | N(C═O)CH2CF3 |
| 6-35 | — | CH2 | CH2 | N(C═O)NCH3 |
| 6-36 | — | CH2 | CH2 | N(C═O)NC2H5 |
| 6-37 | — | CH2 | CH2 | N(C═O)NCH2CCH |
| 6-38 | — | CH2 | N(C═O)CH3 | CH2 |
| 6-39 | — | CH2 | N(C═O)C2H5 | CH2 |
| 6-40 | — | CH2 | N(C═O)CH2CF3 | CH2 |
| 6-41 | — | CH2 | N(C═O)Pr-c | CH2 |
| 6-42 | — | CH2 | N(C═O)NCH3 | CH2 |
| 6-43 | — | CH2 | N(C═O)NC2H5 | CH2 |
| 6-44 | — | CH2 | N(C═O)NCH2CCH | CH2 |
| 6-45 | — | CH2 | O | C═NH |
| 6-46 | — | CH2 | S | C═NH |
| 6-47 | — | CH2 | N | C═NH |
| 6-48 | — | CH | CH | N(C═O)CH3 |
| 6-49 | — | CH | CH | N(C═O)C2H5 |
| 6-50 | — | CH | CH | N(C═O)NC2H5 |
| 6-51 | — | CH | CH | N(C═O)NCH2CCH |
| 6-52 | — | CH2 | CH2 | C═NOH |
| 6-53 | — | CH2 | CH2 | C═NOCH3 |
| 6-54 | — | CH2 | CH2 | C═NOCH2CF3 |
| 6-55 | — | CH2 | CH2 | CHNH2 |
| 6-56 | — | CH2 | CH2 | CHNHCOCH3 |
| 6-57 | — | CH2 | CH2 | CHNH(C═O)C2H5 |
| 6-58 | — | CH2 | CH2 | CHNH(C═O)CH2CF3 |
| 6-59 | — | CH2 | CH2 | CHNHCOPr-c |
| 6-60 | — | CH2 | CH2 | CHNH(C═O)CH2Pr-c |
| 6-61 | — | CH2 | CH2 | CHNH(C═O)CH2SMe |
| 6-62 | — | CH2 | CH2 | CHNH(C═O)CH2S(═O)Me |
| 6-63 | — | CH2 | CH2 | CHNH(C═O)CH2S(═O)2Me |
| 6-64 | — | CH2 | CH2 | CHNHC(═O)NHCH3 |
| 6-65 | — | CH2 | CH2 | CHNHC(═O)NHC2H5 |
| 6-66 | — | CH2 | CH2 | CHNHC(═O)NHCH2CCH |
| 6-67 | — | CH2 | CH2 | CHNHCO2C4H9-tert |
| 6-68 | — | CH2 | CH2 | CHNHCO2CH3 |
| 6-69 | — | CH2 | CH2 | CHNHCO2Ph |
| 6-70 | — | CH2 | CH2 | CHOC(═O)CH3 |
| 6-71 | — | CH2 | CH2 | CHN3 |
| 6-72 | — | CH2 | CH2 | CHNHC(═S)NHC2H5 |
| 6-73 | — | CH2 | CH2 | CHNHCSC2H5 |
| 6-74 | — | CH2 | CHNH2 | CH2 |
| 6-75 | — | CH2 | CHNH(C═O)C2H5 | CH2 |
| 6-76 | — | CH2 | CHNHCO2C4H9-tert | CH2 |
| 6-77 | — | CH2 | C═O | CH2 |
| 6-78 | — | CH2 | C═NOH | CH2 |
| 6-79 | — | O | N | CNH2 |
| 6-80 | — | O | N | CNHCO2C4H9-tert |
| 6-81 | — | NCH3 | N | CNH2 |
| 6-82 | — | NCH3 | N | CNHCO2C4H9-tert |
| 6-83 | — | SO2 | N | CNH2 |
| 6-84 | — | SO2 | N | CNHCO2C4H9-tert |
| 6-85 | — | CH | CH | CHNH2 |
| 6-86 | — | CH | CH | CHNHCOCH3 |
| 6-87 | — | CH | CH | CHNH(C═O)C2H5 |
| 6-88 | — | CH | CH | CHNH(C═O)CH2CF3 |
| 6-89 | — | CH | CH | CHNHCOPr-c |
| 6-90 | — | CH | CH | CHNH(C═O)CH2SMe |
| 6-91 | — | CH | CH | CHNH(C═O)CH2S(═0OMe |
| 6-92 | — | CH | CH | CHNH(C═O)CH2S(═O)2Me |
| 6-93 | — | CH | CH | CHNHC(═O)NHC2H5 |
| 6-94 | — | CH | CH | CHNHCO2C4H9-tert |
| 6-95 | — | CH | CH | CHOC(═O)CH3 |
| 6-96 | — | CH | CH | CHN3 |
| 6-97 | — | CH2 | CH2 | NNH2 |
| 6-98 | — | CH2 | CH2 | NNH(C═O)CH3 |
| 6-99 | — | CH2 | CH2 | NNH(C═O)C2H5 |
| 6-100 | — | CH2 | CH2 | NNH(C═O)CH2CF3 |
| 6-101 | — | CH2 | CH2 | NNH(C═O)Pr-c |
| 6-102 | — | CH2 | CH2 | NNH(C═O)CH2SMe |
| 6-103 | — | CH2 | CH2 | NNH(C═O)CH2S(═O)Me |
| 6-104 | — | CH2 | CH2 | NNH(C═O)CH2S(═O)2Me |
| 6-105 | — | CH2 | CH2 | NNHC(═O)NHC2H5 |
| 6-106 | — | CH2 | CH2 | NNHCO2Bu-t |
| 6-107 | — | CH2 | CHCH3 | CHNHCO2C4H9-tert |
| 6-108 | — | CH2 | C(CH3)2 | CHNHCO2C4H9-tert |
| 6-109 | — | CH2 | CHCl | CHNHCO2C4H9-tert |
| 6-110 | — | CH2 | CHF | CHNHCO2C4H9-tert |
| 6-111 | CH2 | CH2 | O | C═O |
| 6-112 | CH2 | CH2 | O | CH2 |
| 6-113 | CH2 | O | CH2 | CH2 |
| 6-114 | CH2 | CH2 | CH2 | O |
| 6-115 | CH2 | CH | CH | N |
| 6-116 | CH2 | CH | N | CH |
| 6-117 | CH2 | N | CH | CH |
| 6-118 | CH2 | CH | CBr | N |
| 6-119 | CH2 | CH | CCl | N |
| 6-120 | CH2 | CH | CCN | N |
| 6-121 | CH2 | CH2 | CH2 | NH |
| 6-122 | CH2 | CH2 | CH2 | N(C═O)CH3 |
| 6-123 | CH2 | CH2 | CH2 | N(C═O)NC2H5 |
| 6-124 | CH2 | CH2 | NH | CH2 |
| 6-125 | CH2 | CH2 | N(C═O)CH3 | CH2 |
| 6-126 | CH2 | CH2 | N(C═O)NC2H5 | CH2 |
| 6-127 | CH2 | CH2 | C═O | N |
| 6-128 | CH2 | C═O | N | CH2 |
| 6-129 | CH2 | N | C═O | CH2 |
| 6-130 | CH2 | NH | NH | C═O |
| 6-131 | CH2 | NCH3 | NCH3 | C═O |
| 6-132 | CH2 | NCH3 | NH | C═O |
| 6-133 | CH | N | NH | C═O |
| 6-134 | CH2 | O | N | C═O |
| 6-135 | CH2 | N | O | C═O |
| 6-136 | CH | N | O | C═O |
| 6-137 | CH | N | CH | N |
| 6-138 | CH | N | N(C═O)CH3 | C═O |
| 6-139 | CH | N | N(C═O)C2H5 | C═O |
| 6-140 | CH | N | N(C═O)Pr-c | C═O |
| 6-141 | CH | N | N(C═O)CH2CF3 | C═O |
| 6-142 | CH | N | NC(═O)NHC2H5 | C═O |
| 6-143 | CH2 | O | N(C═O)CH3 | C═O |
| 6-144 | CH2 | NCH3 | N(C═O)CH3 | C═O |
| 6-145 | CH2 | CH2 | CH2 | N(C═O)C2H5 |
| 6-146 | CH2 | CH2 | CH2 | N(C═O)CH2CF3 |
| 6-147 | CH2 | CH2 | CH2 | N(C═O)NCH3 |
| 6-148 | CH2 | CH2 | CH2 | N(C═O)NCH2CCH |
| 6-149 | CH2 | CH2 | N(C═O)CH3 | CH2 |
| 6-150 | CH2 | CH2 | N(C═O)C2H5 | CH2 |
| 6-151 | CH2 | CH2 | N(C═O)CH2CF3 | CH2 |
| 6-152 | CH2 | CH2 | N(C═O)Pr-c | CH2 |
| 6-153 | CH2 | CH2 | N(C═O)NCH3 | CH2 |
| 6-154 | CH2 | CH2 | N(C═O)NC2H5 | CH2 |
| 6-155 | CH2 | CH2 | N(C═O)NCH2CCH | CH2 |
| 6-156 | CH2 | CH2 | CH2 | C═O |
| 6-157 | CH2 | CH2 | CH2 | C═NOH |
| 6-158 | CH2 | CH2 | CH2 | C═NOCH3 |
| 6-159 | CH2 | CH2 | CH2 | C═NOCH2CF3 |
| 6-160 | CH2 | CH2 | CH2 | CHNH2 |
| 6-161 | CH2 | CH2 | CH2 | CHNHCOCH3 |
| 6-162 | CH2 | CH2 | CH2 | CHNH(C═O)C2H5 |
| 6-163 | CH2 | CH2 | CH2 | CHNH(C═O)CH2CF3 |
| 6-164 | CH2 | CH2 | CH2 | CHNHCOPr-c |
| 6-165 | CH2 | CH2 | CH2 | CHNH(C═O)CH2Pr-c |
| 6-166 | CH2 | CH2 | CH2 | CHNH(C═O)CH2SMe |
| 6-167 | CH2 | CH2 | CH2 | CHNH(C═O)CH2S(═O)Me |
| 6-168 | CH2 | CH2 | CH2 | CHNH(C═O)CH2S(═O)2Me |
| 6-169 | CH2 | CH2 | CH2 | CHNHC(═O)NHCH3 |
| 6-170 | CH2 | CH2 | CH2 | CHNHC(═O)NHC2H5 |
| 6-171 | CH2 | CH2 | CH2 | CHNHC(═O)NHCH2CCH |
| 6-172 | CH2 | CH2 | CH2 | CHNHCO2C4H9-tert |
| 6-173 | CH2 | CH2 | CH2 | CHNHCO2CH3 |
| 6-174 | CH2 | CH2 | CH2 | CHNHCO2Ph |
| 6-175 | CH2 | CH2 | CH2 | CHOC(═O)CH3 |
| 6-176 | CH2 | CH2 | CH2 | CHN3 |
| 6-177 | CH2 | CH2 | CH2 | CHNHC(═S)NHC2H5 |
| 6-178 | CH2 | CH2 | CH2 | CHNHCSC2H5 |
| 6-179 | CH2 | CH2 | CHNH2 | CH2 |
| 6-180 | CH2 | CH2 | CHNH(C═O)C2H5 | CH2 |
| 6-181 | CH2 | CH2 | CHNHCO2C4H9-tert | CH2 |
| 6-182 | CH2 | CH2 | C═O | CH2 |
| 6-183 | CH2 | CH2 | C═NOH | CH2 |
| 6-184 | CH | CH | CH | CNH2 |
| 6-185 | CH | CH | CH | CNH(C═O)CH3 |
| 6-186 | CH | CH | CH | CNH(C═O)C2H5 |
| 6-187 | CH | CH | CH | CNH(C═O)CH2CF3 |
| 6-188 | CH | CH | CH | CNH(C═O)Pr-c |
| 6-189 | CH | CH | CH | CNH(C═O)CH2Pr-c |
| 6-190 | CH | CH | CH | CNH(C═O)CH2SMe |
| 6-191 | CH | CH | CH | CNH(C═O)CH2S(═O)Me |
| 6-192 | CH | CH | CH | CNH(C═O)CH2S(═O)2Me |
| 6-193 | CH | CH | CH | CNHC(═O)NHCH3 |
| 6-194 | CH | CH | CH | CNHC(═O)NHC2H5 |
| 6-195 | CH | CH | CH | CNHC(═O)NHCH2CCH |
| 6-196 | CH | CH | CH | CNHCO2C4H9-tert |
| 6-197 | CH | CH | CH | CNHCO2CH3 |
| 6-198 | CH | CH | CH | CNHCO2Ph |
| 6-199 | CH | CH | CH | CNC(═S)NHC2H5 |
| 6-200 | CH | CH | CH | CHNHCSC2H5 |
| 6-201 | CH | CH | CNH2 | CH |
| 6-202 | CH | CH | CNH(C═O)CH3 | CH |
| 6-203 | CH | CH | CNH(C═O)C2H5 | CH |
| 6-204 | CH | CH | CNH(C═O)Pr-c | CH |
| 6-205 | CH | CH | CNHC(═O)NHC2H5 | CH |
| 6-206 | CH | CH | CNHCO2C4H9-tert | CH |
| 6-207 | CH | N | N | CCl |
| 6-208 | CH | N | N | CNH2 |
| 6-209 | CH | N | N | CNH(C═O)CH3 |
| 6-210 | CH | N | N | CNH(C═O)C2H5 |
| 6-211 | CH | N | N | CNH(C═O)CH2CF3 |
| 6-212 | CH | N | N | CNH(C═O)Pr-c |
| 6-213 | CH | N | N | CNH(C═O)CH2Pr-c |
| 6-214 | CH | N | N | CNH(C═O)CH2SMe |
| 6-215 | CH | N | N | CNH(C═O)CH2S(═O)Me |
| 6-216 | CH | N | N | CNH(C═O)CH2S(═O)2Me |
| 6-217 | CH | N | N | CNHC(═O)NHCH3 |
| 6-218 | CH | N | N | CNHC(═O)NHC2H5 |
| 6-219 | CH | N | N | CNHC(═O)NHCH2CCH |
| 6-220 | CH | N | N | CNC(═S)NHC2H5 |
| 6-221 | CH | N | N | CNHC(═S)C2H5 |
| 6-222 | CH | CH | N | CNH2 |
| 6-223 | CH | CH | N | CNH(C═O)CH3 |
| 6-224 | CH | CH | N | CNH(C═O)C2H5 |
| 6-225 | CH | CH | N | CNH(C═O)CH2CF3 |
| 6-226 | CH | CH | N | CNH(C═O)Pr-c |
| 6-227 | CH | CH | N | CNHC(═O)NHC2H5 |
| 6-228 | N | CH | CH | CNH2 |
| 6-229 | N | CH | CH | CNH(C═O)C2H5 |
| 6-230 | N | CH | CH | CNHC(═O)NHC2H5 |
| 6-231 | CH | N | CH | CNH2 |
| 6-232 | CH | N | CH | CNH(C═O)Pr-c |
| 6-233 | CH | N | CH | CNH(C═O)CH2S(═O)2Me |
| 6-234 | N | N | CH | CNH2 |
| 6-235 | N | N | CH | CNH(C═O)CH2CF3 |
| 6-236 | N | N | CH | CNH(C═O)CH2SMe |
| 6-237 | N | CH | N | CNH2 |
| 6-238 | N | CH | N | CNH(C═O)CH3 |
| 6-239 | N | CH | N | CNH(C═O)CH2S(═O)Me |
| 6-240 | CH | NCH3 | N | CNH2 |
| 6-241 | CH | NCH3 | N | CNH(C═O)C2H5 |
| 6-242 | CH | NCH3 | N | CNHC(═O)NHC2H5 |
| 6-243 | CH2 | O | N | CNH2 |
| 6-244 | CH2 | O | N | CNH(C═O)Pr-c |
| 6-245 | CH2 | O | N | CNH(C═O)CH2S(═O)2Me |
| 6-246 | CH2 | CH2 | CHCH3 | CHNH2 |
| 6-247 | CH2 | CH2 | CHCH3 | CHNHCOCH3 |
| 6-248 | CH2 | CH2 | CHCH3 | CHNH(C═O)C2H5 |
| 6-249 | CH2 | CH2 | CHCH3 | CHNH(C═O)CH2CF3 |
| 6-250 | CH2 | CH2 | CHCH3 | CHNHCOPr-c |
| 6-251 | CH2 | CH2 | CHCH3 | CHNH(C═O)CH2Pr-c |
| 6-252 | CH2 | CH2 | CHCH3 | CHNH(C═O)CH2SMe |
| 6-253 | CH2 | CH2 | CHCH3 | CHNH(C═O)CH2S(═O)Me |
| 6-254 | CH2 | CH2 | CHCH3 | CHNH(C═O)CH2S(═O)2Me |
| 6-255 | CH2 | CH2 | CHCH3 | CHNHC(═O)NHCH3 |
| 6-256 | CH2 | CH2 | CHCH3 | CHNHC(═O)NHC2H5 |
| 6-257 | CH2 | CH2 | CHCH3 | CHNHC(═O)NHCH2CCH |
| 6-258 | CH2 | CH2 | CHCH3 | CHNHCO2C4H9-tert |
| 6-259 | CH2 | CH2 | CHCH3 | CHNHCO2CH3 |
| 6-260 | CH2 | CH2 | CHCH3 | CHNHCO2Ph |
| 6-261 | CH2 | CH2 | CHCH3 | CHNC(═S)NHC2 H5 |
| 6-262 | CH2 | CH2 | CHCH3 | CHHNH(C═S)C2H5 |
| 6-263 | CH2 | CH2 | C(CH3)2 | CHNH2 |
| 6-264 | CH2 | CH2 | C(CH3)2 | CHNHCOCH3 |
| 6-265 | CH2 | CH2 | C(CH3)2 | CHNHC(═O)NHC2H5 |
| 6-266 | CH2 | CH2 | C(CH3)2 | CHNHCO2C4H9-tert |
| 6-267 | CH2 | CH2 | CHF | CHNH2 |
| 6-268 | CH2 | CH2 | CHF | CHNHCOCH3 |
| 6-269 | CH2 | CH2 | CHF | CHNHC(═O)NHC2H5 |
| 6-270 | CH2 | CH2 | CHF | CHNHCO2C4H9-tert |
| 6-271 | CH2 | CH2 | CHCl | CHNH2 |
| 6-272 | CH2 | CH2 | CHCl | CNH(C═O)CH3 |
| 6-273 | CH2 | CH2 | CHCl | CHNHC(═O)NHC2H5 |
| 6-274 | CH2 | CH2 | CHCl | CHNHCO2C4H9-tert |
| 6-275 | — | CH | CH | NCO2C4H9-tert |

TABLE 6-1

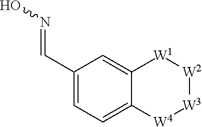

| Example | W¹ | W² | W³ | W⁴ |
|---|---|---|---|---|
| 6-1-1 | — | CH2 | O | C═O |
| 6-1-2 | — | CH2 | O | CH2 |
| 6-1-3 | — | CH2 | CH2 | O |
| 6-1-4 | — | O | CH2 | O |
| 6-1-5 | — | O | CF2 | O |
| 6-1-6 | — | CH | CH | O |
| 6-1-7 | — | CH2 | CH2 | S |
| 6-1-8 | — | CH2 | S | CH2 |
| 6-1-9 | — | CH | CH | S |
| 6-1-10 | — | CH2 | CH2 | NH |
| 6-1-11 | — | CH2 | NH | CH2 |
| 6-1-12 | — | NH | CH2 | CH2 |
| 6-1-13 | — | CH | CH | NH |
| 6-1-14 | — | CH | N | NH |
| 6-1-15 | — | C═O | O | CH2 |
| 6-1-16 | — | CH2 | S | C═O |
| 6-1-17 | — | N | CH | O |
| 6-1-18 | — | N | CH | S |
| 6-1-19 | — | N | CH | NH |
| 6-1-20 | — | O | CH | N |
| 6-1-21 | — | S | CH | N |
| 6-1-22 | — | CH | N | O |
| 6-1-23 | — | CH | N | S |
| 6-1-24 | — | CH2 | CH2 | C═O |
| 6-1-25 | — | CH | CH | C═O |
| 6-1-26 | — | CH2 | C═O | CH2 |
| 6-1-27 | — | C═O | CH2 | CH2 |
| 6-1-28 | — | CH2 | C═O | NH |
| 6-1-29 | — | NH | C═O | CH2 |
| 6-1-30 | — | CH2 | C═O | N(C═O)CH3 |
| 6-1-31 | — | CH2 | C═O | N(C═O)CH2CF3 |
| 6-1-32 | — | CH2 | CH2 | N(C═O)CH3 |
| 6-1-33 | — | CH2 | CH2 | N(C═O)C2H5 |
| 6-1-34 | — | CH2 | CH2 | N(C═O)CH2CF3 |
| 6-1-35 | — | CH2 | CH2 | N(C═O)NCH3 |
| 6-1-36 | — | CH2 | CH2 | N(C═O)NC2H5 |
| 6-1-37 | — | CH2 | CH2 | N(C═O)NCH2CCH |
| 6-1-38 | — | CH2 | N(C═O)CH3 | CH2 |
| 6-1-39 | — | CH2 | N(C═O)C2H5 | CH2 |
| 6-1-40 | — | CH2 | N(C═O)CH2CF3 | CH2 |
| 6-1-41 | — | CH2 | N(C═O)Pr-c | CH2 |
| 6-1-42 | — | CH2 | N(C═O)NCH3 | CH2 |
| 6-1-43 | — | CH2 | N(C═O)NC2H5 | CH2 |
| 6-1-44 | — | CH2 | N(C═O)NCH2CCH | CH2 |
| 6-1-45 | — | CH2 | O | C═NH |
| 6-1-46 | — | CH2 | S | C═NH |
| 6-1-47 | — | CH2 | N | C═NH |
| 6-1-48 | — | CH | CH | N(C═O)CH3 |
| 6-1-49 | — | CH | CH | N(C═O)C2H5 |
| 6-1-50 | — | CH | CH | N(C═O)NC2H5 |
| 6-1-51 | — | CH | CH | N(C═O)NCH2CCH |
| 6-1-52 | — | CH2 | CH2 | C═NOH |
| 6-1-53 | — | CH2 | CH2 | C═NOCH3 |
| 6-1-54 | — | CH2 | CH2 | C═NOCH2CF3 |
| 6-1-55 | — | CH2 | CH2 | CHNH2 |
| 6-1-56 | — | CH2 | CH2 | CHNH(C═O)CH3 |
| 6-1-57 | — | CH2 | CH2 | CHNH(C═O)C2H5 |
| 6-1-58 | — | CH2 | CH2 | CHNH(C═O)CH2CF3 |
| 6-1-59 | — | CH2 | CH2 | CHNH(C═O)Pr-c |
| 6-1-60 | — | CH2 | CH2 | CHNH(C═O)CH2Pr-c |
| 6-1-61 | — | CH2 | CH2 | CHNH(C═O)CH2SMe |
| 6-1-62 | — | CH2 | CH2 | CHNH(C═)OCH2S(═O)Me |
| 6-1-63 | — | CH2 | CH2 | CHNH(C═O)CH2S(═O)2Me |
| 6-1-64 | — | CH2 | CH2 | CHNHC(═O)NHCH3 |
| 6-1-65 | — | CH2 | CH2 | CHNHC(═O)NHC2H5 |
| 6-1-66 | — | CH2 | CH2 | CHNHC(═O)NHCH2CCH |
| 6-1-67 | — | CH2 | CH2 | CHNHCO2Bu-t |
| 6-1-68 | — | CH2 | CH2 | CHNHCO2CH3 |
| 6-1-69 | — | CH2 | CH2 | CHNHCO2Ph |
| 6-1-70 | — | CH2 | CH2 | CHOC(═O)CH3 |
| 6-1-71 | — | CH2 | CH2 | CHN3 |
| 6-1-72 | — | CH2 | CH2 | CHNHC(═S)NHC2H5 |
| 6-1-73 | — | CH2 | CH2 | CHNH(C═S)C2H5 |
| 6-1-74 | — | CH2 | CHNH2 | CH2 |
| 6-1-75 | — | CH2 | CHNH(C═O)C2H5 | CH2 |
| 6-1-76 | — | CH2 | CHNHCO2Bu-t | CH2 |
| 6-1-77 | — | CH2 | C═O | CH2 |
| 6-1-78 | — | CH2 | C═NOH | CH2 |
| 6-1-79 | — | O | N | CNH2 |
| 6-1-80 | — | O | N | CNHCO2Bu-t |
| 6-1-81 | — | NCH3 | N | CNH2 |
| 6-1-82 | — | NCH3 | N | CNHCO2Bu-t |
| 6-1-83 | — | SO2 | N | CNH2 |
| 6-1-84 | — | SO2 | N | CNHCO2Bu-t |
| 6-1-85 | — | CH | CH | CHNH2 |
| 6-1-86 | — | CH | CH | CHNH(C═O)CH3 |
| 6-1-87 | — | CH | CH | CHNH(C═O)C2H5 |
| 6-1-88 | — | CH | CH | CHNH(C═O)CH2CF3 |
| 6-1-89 | — | CH | CH | CHNH(C═O)Pr-c |
| 6-1-90 | — | CH | CH | CHNH(C═O)CH2SMe |
| 6-1-91 | — | CH | CH | CHNH(C═)OCH2S(═O)Me |
| 6-1-92 | — | CH | CH | CHNH(C═O)CH2S(═O)2Me |
| 6-1-93 | — | CH | CH | CHNHC(═O)NHC2H5 |
| 6-1-94 | — | CH | CH | CHNHCO2Bu-t |
| 6-1-95 | — | CH | CH | CHOC(═O)CH3 |
| 6-1-96 | — | CH | CH | CHN3 |
| 6-1-97 | — | CH2 | CH2 | NNH2 |
| 6-1-98 | — | CH2 | CH2 | NNH(C═O)CH3 |
| 6-1-99 | — | CH2 | CH2 | NNH(C═O)C2H5 |
| 6-1-100 | — | CH2 | CH2 | NNH(C═O)CH2CF3 |
| 6-1-101 | — | CH2 | CH2 | NNH(C═O)Pr-c |
| 6-1-102 | — | CH2 | CH2 | NNH(C═O)CH2SMe |
| 6-1-103 | — | CH2 | CH2 | NNH(C═O)CH2S(═O)Me |
| 6-1-104 | — | CH2 | CH2 | NNH(C═O)CH2S(═O)2Me |
| 6-1-105 | — | CH2 | CH2 | NNHC(═O)NHC2H5 |
| 6-1-106 | — | CH2 | CH2 | NNHCO2Bu-t |
| 6-1-107 | — | CH2 | CHCH3 | CHNHCO2Bu-t |
| 6-1-108 | — | CH2 | C(CH3)2 | CHNHCO2Bu-t |
| 6-1-109 | — | CH2 | CHCl | CHNHCO2Bu-t |
| 6-1-110 | — | CH2 | CHF | CHNHCO2Bu-t |
| 6-1-111 | CH2 | CH2 | O | C═O |
| 6-1-112 | CH2 | CH2 | O | CH2 |
| 6-1-113 | CH2 | O | CH2 | CH2 |
| 6-1-114 | CH2 | CH2 | CH2 | O |
| 6-1-115 | CH2 | CH | CH | N |
| 6-1-116 | CH2 | CH | N | CH |
| 6-1-117 | CH2 | N | CH | CH |
| 6-1-118 | CH2 | CH | CBr | N |
| 6-1-119 | CH2 | CH | CCl | N |
| 6-1-120 | CH2 | CH | CCN | N |
| 6-1-121 | CH2 | CH2 | CH2 | NH |
| 6-1-122 | CH2 | CH2 | CH2 | N(C═O)CH3 |
| 6-1-123 | CH2 | CH2 | CH2 | N(C═O)NC2H5 |
| 6-1-124 | CH2 | CH2 | NH | CH2 |
| 6-1-125 | CH2 | CH2 | N(C═O)CH3 | CH2 |
| 6-1-126 | CH2 | CH2 | N(C═O)NC2H5 | CH2 |
| 6-1-127 | CH2 | CH2 | C═O | N |
| 6-1-128 | CH2 | C═O | N | CH2 |
| 6-1-129 | CH2 | N | C═O | CH2 |
| 6-1-130 | CH2 | NH | NH | C═O |
| 6-1-131 | CH2 | NCH3 | NCH3 | C═O |
| 6-1-132 | CH2 | NCH3 | NH | C═O |
| 6-1-133 | CH | N | NH | C═O |
| 6-1-134 | CH2 | O | N | C═O |
| 6-1-135 | CH2 | N | O | C═O |
| 6-1-136 | CH | N | O | C═O |
| 6-1-137 | CH | N | CH | N |
| 6-1-138 | CH | N | N(C═O)CH3 | C═O |
| 6-1-139 | CH | N | N(C═O)C2H5 | C═O |
| 6-1-140 | CH | N | N(C═O)Pr-c | C═O |
| 6-1-141 | CH | N | N(C═O)CH2CF3 | C═O |
| 6-1-142 | CH | N | NC(═O)NHC2H5 | C═O |
| 6-1-143 | CH2 | O | N(C═O)CH3 | C═O |
| 6-1-144 | CH2 | NCH3 | N(C═O)CH3 | C═O |
| 6-1-145 | CH2 | CH2 | CH2 | N(C═O)C2H5 |
| 6-1-146 | CH2 | CH2 | CH2 | N(C═O)CH2CF3 |
| 6-1-147 | CH2 | CH2 | CH2 | N(C═O)NCH3 |
| 6-1-148 | CH2 | CH2 | CH2 | N(C═O)NCH2CCH |
| 6-1-149 | CH2 | CH2 | N(C═O)CH3 | CH2 |
| 6-1-150 | CH2 | CH2 | N(C═O)C2H5 | CH2 |
| 6-1-151 | CH2 | CH2 | N(C═O)CH2CF3 | CH2 |
| 6-1-152 | CH2 | CH2 | N(C═O)Pr-c | CH2 |
| 6-1-153 | CH2 | CH2 | N(C═O)NCH3 | CH2 |
| 6-1-154 | CH2 | CH2 | N(C═O)NC2H5 | CH2 |
| 6-1-155 | CH2 | CH2 | N(C═O)NCH2CCH | CH2 |
| 6-1-156 | CH2 | CH2 | CH2 | C═O |
| 6-1-157 | CH2 | CH2 | CH2 | C═NOH |
| 6-1-158 | CH2 | CH2 | CH2 | C═NOCH3 |
| 6-1-159 | CH2 | CH2 | CH2 | C═NOCH2CF3 |
| 6-1-160 | CH2 | CH2 | CH2 | CHNH2 |
| 6-1-161 | CH2 | CH2 | CH2 | CHNH(C═O)CH3 |
| 6-1-162 | CH2 | CH2 | CH2 | CHNH(C═O)C2H5 |
| 6-1-163 | CH2 | CH2 | CH2 | CHNH(C═O)CH2CF3 |
| 6-1-164 | CH2 | CH2 | CH2 | CHNH(C═O)Pr-c |
| 6-1-165 | CH2 | CH2 | CH2 | CHNH(C═O)CH2Pr-c |
| 6-1-166 | CH2 | CH2 | CH2 | CHNH(C═O)CH2SMe |
| 6-1-167 | CH2 | CH2 | CH2 | CHNH(C═)OCH2S(═O)Me |
| 6-1-168 | CH2 | CH2 | CH2 | CHNH(C═O)CH2S(═O)2Me |
| 6-1-169 | CH2 | CH2 | CH2 | CHNHC(═O)NHCH3 |
| 6-1-170 | CH2 | CH2 | CH2 | CHNHC(═O)NHC2H5 |
| 6-1-171 | CH2 | CH2 | CH2 | CHNHC(═O)NHCH2CCH |
| 6-1-172 | CH2 | CH2 | CH2 | CHNHCO2Bu-t |
| 6-1-173 | CH2 | CH2 | CH2 | CHNHCO2CH3 |
| 6-1-174 | CH2 | CH2 | CH2 | CHNHCO2Ph |
| 6-1-175 | CH2 | CH2 | CH2 | CHOC(═O)CH3 |
| 6-1-176 | CH2 | CH2 | CH2 | CHN3 |
| 6-1-177 | CH2 | CH2 | CH2 | CHNHC(═S)NHC2H5 |
| 6-1-178 | CH2 | CH2 | CH2 | CHNH(C═S)C2H5 |
| 6-1-179 | CH2 | CH2 | CHNH2 | CH2 |
| 6-1-180 | CH2 | CH2 | CHNH(C═O)C2H5 | CH2 |
| 6-1-181 | CH2 | CH2 | CHNHCO2Bu-t | CH2 |
| 6-1-182 | CH2 | CH2 | C═O | CH2 |
| 6-1-183 | CH2 | CH2 | C═NOH | CH2 |
| 6-1-184 | CH | CH | CH | CNH2 |
| 6-1-185 | CH | CH | CH | CNH(C═O)CH3 |
| 6-1-186 | CH | CH | CH | CNH(C═O)C2H5 |
| 6-1-187 | CH | CH | CH | CNH(C═O)CH2CF3 |
| 6-1-188 | CH | CH | CH | CNH(C═O)Pr-c |
| 6-1-189 | CH | CH | CH | CNH(C═O)CH2Pr-c |
| 6-1-190 | CH | CH | CH | CNH(C═O)CH2SMe |
| 6-1-191 | CH | CH | CH | CNH(C═O)CH2S(═O)Me |
| 6-1-192 | CH | CH | CH | CNH(C═O)CH2S(═O)2Me |
| 6-1-193 | CH | CH | CH | CNHC(═O)NHCH3 |
| 6-1-194 | CH | CH | CH | CNH(C═O)NHC2H5 |
| 6-1-195 | CH | CH | CH | CNHC(═O)NHCH2CCH |
| 6-1-196 | CH | CH | CH | CNHCO2Bu-t |
| 6-1-197 | CH | CH | CH | CNHCO2CH3 |
| 6-1-198 | CH | CH | CH | CNHCO2Ph |
| 6-1-199 | CH | CH | CH | CNC(═S)NHC2H5 |
| 6-1-200 | CH | CH | CH | CHNH(C═S)C2H5 |
| 6-1-201 | CH | CH | CNH2 | CH |
| 6-1-202 | CH | CH | CNH(C═O)CH3 | CH |
| 6-1-203 | CH | CH | CNH(C═O)C2H5 | CH |
| 6-1-204 | CH | CH | CNH(C═O)Pr-c | CH |
| 6-1-205 | CH | CH | CNHC(═O)NHC2H5 | CH |
| 6-1-206 | CH | N | N | CCI |
| 6-1-207 | CH | N | N | CNH2 |
| 6-1-208 | CH | N | N | CNH(C═O)CH3 |
| 6-1-209 | CH | N | N | CNH(C═O)C2H5 |
| 6-1-210 | CH | N | N | CNH(C═O)CH2CF3 |
| 6-1-211 | CH | N | N | CNH(C═O)Pr-c |
| 6-1-212 | CH | N | N | CNH(C═O)CH2Pr-c |
| 6-1-213 | CH | N | N | CNH(C═O)CH2SMe |
| 6-1-214 | CH | N | N | CNH(C═O)CH2S(═0)Me |
| 6-1-215 | CH | N | N | CNH(C═O)CH2S(═O)2Me |
| 6-1-216 | CH | N | N | CNHC(═O)NHCH3 |
| 6-1-217 | CH | N | N | CNH(C═O)NHC2H5 |
| 6-1-218 | CH | N | N | CNHC(═O)NHCH2CCH |
| 6-1-219 | CH | N | N | CNC(═S)NHC2H5 |
| 6-1-220 | CH | N | N | CNHC(═S)C2H5 |
| 6-1-221 | CH | CH | N | CNH2 |
| 6-1-222 | CH | CH | N | CNH(C═O)CH3 |
| 6-1-223 | CH | CH | N | CNH(C═O)C2H5 |
| 6-1-224 | CH | CH | N | CNH(C═O)CH2CF3 |
| 6-1-225 | CH | CH | N | CNH(C═0)Pr-c |
| 6-1-226 | CH | CH | N | CNH(C═O)NHC2H5 |
| 6-1-227 | N | CH | CH | CNH2 |
| 6-1-228 | N | CH | CH | CNH(C═O)C2H5 |
| 6-1-229 | N | CH | CH | CNH(C═O)NHC2H5 |
| 6-1-230 | CH | N | CH | CNH2 |
| 6-1-231 | CH | N | CH | CNH(C═O)Pr-c |
| 6-1-232 | CH | N | CH | CNH(C═O)CH2S(═O)2Me |
| 6-1-233 | N | N | CH | CNH2 |
| 6-1-234 | N | N | CH | CNH(C═O)CH2CF3 |
| 6-1-235 | N | N | CH | CNH(C═O)CH2SMe |
| 6-1-236 | N | CH | N | CNH2 |
| 6-1-237 | N | CH | N | CNH(C═O)CH3 |
| 6-1-238 | N | CH | N | CNH(C═O)CH2S(═O)Me |
| 6-1-239 | CH | NCH3 | N | CNH2 |
| 6-1-240 | CH | NCH3 | N | CNH(C═O)C2H5 |
| 6-1-241 | CH | NCH3 | N | CNH(C═O)NHC2H5 |
| 6-1-242 | CH2 | O | N | CNH2 |
| 6-1-243 | CH2 | O | N | CNH(C═O)Pr-c |
| 6-1-244 | CH2 | O | N | CNH(C═O)CH2S(═O)2Me |
| 6-1-245 | CH2 | CH2 | CHCH3 | CNH2 |
| 6-1-246 | CH2 | CH2 | CHCH3 | CNH(C═O)CH3 |
| 6-1-247 | CH2 | CH2 | CHCH3 | CNH(C═O)C2H5 |
| 6-1-248 | CH2 | CH2 | CHCH3 | CNH(C═O)CH2CF3 |
| 6-1-249 | CH2 | CH2 | CHCH3 | CNH(C═O)Pr-c |
| 6-1-250 | CH2 | CH2 | CHCH3 | CNH(C═O)CH2Pr-c |
| 6-1-251 | CH2 | CH2 | CHCH3 | CNH(C═O)CH2SMe |
| 6-1-252 | CH2 | CH2 | CHCH3 | CNH(C═O)CH2S(═O)Me |
| 6-1-253 | CH2 | CH2 | CHCH3 | CNH(C═O)CH2S(═O)2Me |
| 6-1-254 | CH2 | CH2 | CHCH3 | CNHC(═O)NHCH3 |
| 6-1-255 | CH2 | CH2 | CHCH3 | CNH(C═O)NHC2H5 |
| 6-1-256 | CH2 | CH2 | CHCH3 | CNHC(═O)NHCH2CCH |
| 6-1-257 | CH2 | CH2 | CHCH3 | CNHCO2Bu-t |
| 6-1-258 | CH2 | CH2 | CHCH3 | CNHCO2CH3 |
| 6-1-259 | CH2 | CH2 | CHCH3 | CNHCO2Ph |
| 6-1-260 | CH2 | CH2 | CHCH3 | CNC(═S)NHC2H5 |
| 6-1-261 | CH2 | CH2 | CHCH3 | CHNH(C═S)C2H5 |
| 6-1-262 | CH2 | CH2 | C(CH3)2 | CNH2 |
| 6-1-263 | CH2 | CH2 | C(CH3)2 | CNH(C═O)CH3 |
| 6-1-264 | CH2 | CH2 | C(CH3)2 | CNH(C═O)NHC2H5 |
| 6-1-265 | CH2 | CH2 | C(CH3)2 | CNHCO2Bu-t |
| 6-1-266 | CH2 | CH2 | CHF | CNH2 |
| 6-1-267 | CH2 | CH2 | CHF | CNH(C═O)CH3 |
| 6-1-268 | CH2 | CH2 | CHF | CNH(C═O)NHC2H5 |
| 6-1-269 | CH2 | CH2 | CHF | CNHCO2Bu-t |
| 6-1-270 | CH2 | CH2 | CHCl | CNH2 |
| 6-1-271 | CH2 | CH2 | CHCl | CNH(C═O)CH3 |
| 6-1-272 | CH2 | CH2 | CHCl | CNH(C═O)NHC2H5 |
| 6-1-273 | CH2 | CH2 | CHCl | CNHCO2Bu-t |

TABLE 6-2

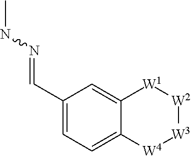

| Example | W¹ | W² | W³ | W⁴ |
|---|---|---|---|---|
| 6-2-1 | — | CH2 | O | C═O |
| 6-2-2 | — | CH2 | O | CH2 |
| 6-2-3 | — | CH2 | CH2 | O |
| 6-2-4 | — | O | CH2 | O |
| 6-2-5 | — | O | CF2 | O |
| 6-2-6 | — | CH | CH | O |
| 6-2-7 | — | CH2 | CH2 | S |
| 6-2-8 | — | CH2 | S | CH2 |
| 6-2-9 | — | CH | CH | S |
| 6-2-10 | — | CH2 | CH2 | NH |
| 6-2-11 | — | CH2 | NH | CH2 |
| 6-2-12 | — | NH | CH2 | CH2 |
| 6-2-13 | — | CH | CH | NH |
| 6-2-14 | — | CH | N | NH |
| 6-2-15 | — | C═O | O | CH2 |
| 6-2-16 | — | CH2 | S | C═O |
| 6-2-17 | — | N | CH | O |
| 6-2-18 | — | N | CII | S |
| 6-2-19 | — | N | CH | NH |
| 6-2-20 | — | O | CH | N |
| 6-2-21 | — | S | CH | N |
| 6-2-22 | — | CH | N | O |
| 6-2-23 | — | CH | N | S |
| 6-2-24 | — | CH2 | CH2 | C═O |
| 6-2-25 | — | CH | CH | C═O |
| 6-2-26 | — | CH2 | C═O | CH2 |
| 6-2-27 | — | C═O | CH2 | CH2 |
| 6-2-28 | — | CH2 | C═O | NH |
| 6-2-29 | — | NH | C═O | CH2 |
| 6-2-30 | — | CH2 | C═O | N(C═O)CH3 |
| 6-2-31 | — | CH2 | C═O | N(C═O)CH2CF3 |
| 6-2-32 | — | CH2 | CH2 | N(C═O)CH3 |
| 6-2-33 | — | CH2 | CH2 | N(C═O)C2H5 |
| 6-2-34 | — | CH2 | CH2 | N(C═O)CH2CF3 |
| 6-2-35 | — | CH2 | CH2 | N(C═O)NCH3 |
| 6-2-36 | — | CH2 | CH2 | N(C═O)NC2H5 |
| 6-2-37 | — | CH2 | CH2 | N(C═O)NCH2CCH |
| 6-2-38 | — | CH2 | N(C═O)CH3 | CH2 |
| 6-2-39 | — | CH2 | N(C═O)C2H5 | CH2 |
| 6-2-40 | — | CH2 | N(C═O)CH2CF3 | CH2 |
| 6-2-41 | — | CH2 | N(C═O)Pr-c | CH2 |
| 6-2-42 | — | CH2 | N(C═O)NCH3 | CH2 |
| 6-2-43 | — | CH2 | N(C═O)NC2H5 | CH2 |
| 6-2-44 | — | CH2 | N(C═O)NCH2CCH | CH2 |
| 6-2-45 | — | CH2 | O | C═NH |
| 6-2-46 | — | CH2 | S | C═NH |
| 6-2-47 | — | CH2 | N | C═NH |
| 6-2-48 | — | CH | CH | N(C═O)CH3 |
| 6-2-49 | — | CH | CH | N(C═O)C2H5 |
| 6-2-50 | — | CH | CH | N(C═O)NC2H5 |
| 6-2-51 | — | CH | CH | N(C═O)NCH2CCH |
| 6-2-52 | — | CH2 | CH2 | C═NOH |
| 6-2-53 | — | CH2 | CH2 | C═NOCH3 |
| 6-2-54 | — | CH2 | CH2 | C═NOCH2CF3 |
| 6-2-55 | — | CH2 | CH2 | CHNH2 |
| 6-2-56 | — | CH2 | CH2 | CHNH(C═O)CH3 |
| 6-2-57 | — | CH2 | CH2 | CHNH(C═O)C2H5 |
| 6-2-58 | — | CH2 | CH2 | CHNH(C═O)CH2CF3 |
| 6-2-59 | — | CH2 | CH2 | CHNH(C═O)Pr-c |
| 6-2-60 | — | CH2 | CH2 | CHNH(C═O)CH2Pr-c |
| 6-2-61 | — | CH2 | CH2 | CHNH(C═O)CH2SMe |
| 6-2-62 | — | CH2 | CH2 | CHNH(C═)OCH2S(═O)Me |
| 6-2-63 | — | CH2 | CH2 | CHNH(C═O)CH2S(═O)2Me |
| 6-2-64 | — | CH2 | CH2 | CHNHC(═O)NHCH3 |
| 6-2-65 | — | CH2 | CH2 | CHNHC(═O)NHC2H5 |
| 6-2-66 | — | CH2 | CH2 | CHNHC(═O)NHCH2CCH |
| 6-2-67 | — | CH2 | CH2 | CHNHCO2Bu-t |
| 6-2-68 | — | CH2 | CH2 | CHNHCO2CH3 |
| 6-2-69 | — | CH2 | CH2 | CHNHCO2Ph |
| 6-2-70 | — | CH2 | CH2 | CHOC(═O)CH3 |
| 6-2-71 | — | CH2 | CH2 | CHN3 |
| 6-2-72 | — | CH2 | CH2 | CHNHC(═S)NHC2H5 |
| 6-2-73 | — | CH2 | CH2 | CHNH(C═S)C2H5 |
| 6-2-74 | — | CH2 | CHNH2 | CH2 |
| 6-2-75 | — | CH2 | CHNH(C═O)C2H5 | CH2 |
| 6-2-76 | — | CH2 | CHNHCO2Bu-t | CH2 |
| 6-2-77 | — | CH2 | C═O | CH2 |
| 6-2-78 | — | CH2 | C═NOH | CH2 |
| 6-2-79 | — | O | N | CNH2 |
| 6-2-80 | — | O | N | CNHCO2Bu-t |
| 6-2-81 | — | NCH3 | N | CNH2 |
| 6-2-82 | — | NCH3 | N | CNHCO2Bu-t |
| 6-2-83 | — | SO2 | N | CNH2 |
| 6-2-84 | — | SO2 | N | CNHCO2Bu-t |
| 6-2-85 | — | CH | CH | CHNH2 |
| 6-2-86 | — | CH | CH | CHNH(C═O)CH3 |
| 6-2-87 | — | CH | CH | CHNH(C═O)C2H5 |
| 6-2-88 | — | CH | CH | CHNH(C═O)CH2CF3 |
| 6-2-89 | — | CH | CH | CHNH(C═O)Pr-c |
| 6-2-90 | — | CH | CH | CHNH(C═O)CH2SMe |
| 6-2-91 | — | CH | CH | CHNH(C═)OCH2S(═O)Me |
| 6-2-92 | — | CH | CH | CHNH(C═O)CH2S(═O)2Me |
| 6-2-93 | — | CH | CH | CHNHC(═O)NHC2H5 |
| 6-2-94 | — | CH | CH | CHNHCO2Bu-t |
| 6-2-95 | — | CH | CH | CHOC(═O)CH3 |
| 6-2-96 | — | CH | CH | CHN3 |
| 6-2-97 | — | CH2 | CH2 | NNH2 |
| 6-2-98 | — | CH2 | CH2 | NM-I(C═O)CH3 |
| 6-2-99 | — | CH2 | CH2 | NNH(C═O)C2H5 |
| 6-2-100 | — | CH2 | CH2 | NNH(C═O)CH2CF3 |
| 6-2-101 | — | CH2 | CH2 | NNH(C═O)Pr-c |
| 6-2-102 | — | CH2 | CH2 | NNH(C═O)CH2SMe |
| 6-2-103 | — | CH2 | CH2 | NNH(C═O)CH2S(═O)Me |
| 6-2-104 | — | CH2 | CH2 | NNH(C═O)CH2S(═O)2Me |
| 6-2-105 | — | CH2 | CH2 | NNHC(═O)NHC2H5 |
| 6-2-106 | — | CH2 | CH2 | NNHCO2Bu-t |
| 6-2-107 | — | CH2 | CHCH3 | CHNHCO2Bu-t |
| 6-2-108 | — | CH2 | C(CH3)2 | CHNHCO2Bu-t |
| 6-2-109 | — | CH2 | CHCl | CHNHCO2Bu-t |
| 6-2-110 | — | CH2 | CHF | CHNHCO2Bu-t |
| 6-2-111 | CH2 | CH2 | O | C═O |
| 6-2-112 | CH2 | CH2 | O | CH2 |
| 6-2-113 | CH2 | O | CH2 | CH2 |
| 6-2-114 | CH2 | CH2 | CH2 | O |
| 6-2-115 | CH2 | CH | CH | N |
| 6-2-116 | CH2 | CH | N | CH |
| 6-2-117 | CH2 | N | CH | CH |
| 6-2-118 | CH2 | CH | CBr | N |
| 6-2-119 | CH2 | CH | CCl | N |
| 6-2-120 | CH2 | CH | CCN | N |
| 6-2-121 | CH2 | CH2 | CH2 | NH |
| 6-2-122 | CH2 | CH2 | CH2 | N(C═O)CH3 |
| 6-2-123 | CH2 | CH2 | CH2 | N(C═O)NC2H5 |
| 6-2-124 | CH2 | CH2 | NH | CH2 |
| 6-2-125 | CH2 | CH2 | N(C═O)CH3 | CH2 |
| 6-2-126 | CH2 | CH2 | N(C═O)NC2H5 | CH2 |
| 6-2-127 | CH2 | CH2 | C═O | N |
| 6-2-128 | CH2 | C═O | N | CH2 |
| 6-2-129 | CH2 | N | C═O | CH2 |
| 6-2-130 | CH2 | NH | NH | C═O |
| 6-2-131 | CH2 | NCH3 | NCH3 | C═O |
| 6-2-132 | CH2 | NCH3 | NH | C═O |
| 6-2-133 | CH | N | NH | C═O |
| 6-2-134 | CH2 | O | N | C═O |
| 6-2-135 | CH2 | N | O | C═O |
| 6-2-136 | CH | N | O | C═O |
| 6-2-137 | CH | N | CH | N |
| 6-2-138 | CH | N | N(C═O)CH3 | C═O |
| 6-2-139 | CH | N | N(C═O)C2H5 | C═O |
| 6-2-140 | CH | N | N(C═O)Pr-c | C═O |
| 6-2-141 | CH | N | N(C═O)CH2CF3 | C═O |
| 6-2-142 | CH | N | NC(═O)NHC2H5 | C═O |
| 6-2-143 | CH2 | O | N(C═O)CH3 | C═O |
| 6-2-144 | CH2 | NCH3 | N(C═O)CH3 | C═O |
| 6-2-145 | CH2 | CH2 | CH2 | N(C═O)C2H5 |
| 6-2-146 | CH2 | CH2 | CH2 | N(C═O)CH2CF3 |
| 6-2-147 | CH2 | CH2 | CH2 | N(C═O)NCH3 |
| 6-2-148 | CH2 | CH2 | CH2 | N(C═O)NCH2CCH |
| 6-2-149 | CH2 | CH2 | N(C═O)CH3 | CH2 |
| 6-2-150 | CH2 | CH2 | N(C═O)C2H5 | CH2 |
| 6-2-151 | CH2 | CH2 | N(C═O)CH2CF3 | CH2 |
| 6-2-152 | CH2 | CH2 | N(C═O)Pr-c | CH2 |
| 6-2-153 | CH2 | CH2 | N(C═O)NCH3 | CH2 |
| 6-2-154 | CH2 | CH2 | N(C═O)NC2H5 | CH2 |
| 6-2-155 | CH2 | CH2 | N(C═O)NCH2CCH | CH2 |
| 6-2-156 | CH2 | CH2 | CH2 | C═O |
| 6-2-157 | CH2 | CH2 | CH2 | C═NOH |
| 6-2-158 | CH2 | CH2 | CH2 | C═NOCH3 |
| 6-2-159 | CH2 | CH2 | CH2 | C═NOCH2CF3 |
| 6-2-160 | CH2 | CH2 | CH2 | CHNH2 |
| 6-2-161 | CH2 | CH2 | CH2 | CHNH(C═O)CH3 |
| 6-2-162 | CH2 | CH2 | CH2 | CHNH(C═O)C2H5 |
| 6-2-163 | CH2 | CH2 | CH2 | CHNH(C═O)CH2CF3 |
| 6-2-164 | CH2 | CH2 | CH2 | CHNH(C═O)Pr-c |
| 6-2-165 | CH2 | CH2 | CH2 | CHNH(C═O)CH2Pr-c |
| 6-2-166 | CH2 | CH2 | CH2 | CHNH(C═O)CH2SMe |
| 6-2-167 | CH2 | CH2 | CH2 | CHNH(C═)OCH2S(═O)Me |
| 6-2-168 | CH2 | CH2 | CH2 | CHNH(C═O)CH2S(═O)2Me |
| 6-2-169 | CH2 | CH2 | CH2 | CHNHC(═O)NHCH3 |
| 6-2-170 | CH2 | CH2 | CH2 | CHNHC(═O)NHC2H5 |
| 6-2-171 | CH2 | CH2 | CH2 | CHNHC(═O)NHCH2CCH |
| 6-2-172 | CH2 | CH2 | CH2 | CHNHCO2Bu-t |
| 6-2-173 | CH2 | CH2 | CH2 | CHNHCO2CH3 |
| 6-2-174 | CH2 | CH2 | CH2 | CHNHCO2Ph |
| 6-2-175 | CH2 | CH2 | CH2 | CHOC(═O)CH3 |
| 6-2-176 | CH2 | CH2 | CH2 | CHN3 |
| 6-2-177 | CH2 | CH2 | CH2 | CHNHC(═S)NHC2H5 |
| 6-2-178 | CH2 | CH2 | CH2 | CHNH(C═S)C2H5 |
| 6-2-179 | CH2 | CH2 | CHNH2 | CH2 |
| 6-2-180 | CH2 | CH2 | CHNH(C═O)C2H5 | CH2 |
| 6-2-181 | CH2 | CH2 | CHNHCO2Bu-t | CH2 |
| 6-2-182 | CH2 | CH2 | C═O | CH2 |
| 6-2-183 | CH2 | CH2 | C═NOH | CH2 |
| 6-2-184 | CH | CH | CH | CNH2 |
| 6-2-185 | CH | CH | CH | CNH(C═O)CH3 |
| 6-2-186 | CH | CH | CH | CNH(C═O)C2H5 |
| 6-2-187 | CH | CH | CH | CNH(C═O)CH2CF3 |
| 6-2-188 | CH | CH | CH | CNH(C═O)Pr-c |
| 6-2-189 | CH | CH | CH | CNH(C═O)CH2Pr-c |
| 6-2-190 | CH | CH | CH | CNH(C═O)CH2SMe |
| 6-2-191 | CH | CH | CH | CNH(C═O)CH2S(═O)Me |
| 6-2-192 | CH | CH | CH | CNH(C═O)CH2S(═O)2Me |
| 6-2-193 | CH | CH | CH | CNHC(═O)NHCH3 |
| 6-2-194 | CH | CH | CH | CNH(C═O)NHC2H5 |
| 6-2-195 | CH | CH | CH | CNHC(═O)NHCH2CCH |
| 6-2-196 | CH | CH | CH | CNHCO2Bu-t |
| 6-2-197 | CH | CH | CH | CNHCO2CH3 |
| 6-2-198 | CH | CH | CH | CNHCO2Ph |
| 6-2-199 | CH | CH | CH | CNC(═S)NHC2H5 |
| 6-2-200 | CH | CH | CH | CHNH(C═S)C2H5 |
| 6-2-201 | CH | CH | CNH2 | CH |
| 6-2-202 | CH | CH | CNH(C═O)CH3 | CH |
| 6-2-203 | CH | CH | CNH(C═O)C2H5 | CH |
| 6-2-204 | CH | CH | CNH(C═O)Pr-c | CH |
| 6-2-205 | CH | CH | CNHC(═O)NHC2H5 | CH |
| 6-2-206 | CH | N | N | CCl |
| 6-2-207 | CH | N | N | CNH2 |
| 6-2-208 | CH | N | N | CNH(C═O)CH3 |
| 6-2-209 | CH | N | N | CNH(C═O)C2H5 |
| 6-2-210 | CH | N | N | CNH(C═O)CH2CF3 |
| 6-2-211 | CH | N | N | CNH(C═O)Pr-c |
| 6-2-212 | CH | N | N | CNH(C═O)CH2Pr-c |
| 6-2-213 | CH | N | N | CNH(C═O)CH2 SMe |
| 6-2-214 | CH | N | N | CNH(C═O)CH2S(═O)Me |
| 6-2-215 | CH | N | N | CNH(C═O)CH2S(═O)2Me |
| 6-2-216 | CH | N | N | CNHC(═O)NHCH3 |
| 6-2-217 | CH | N | N | CNH(C═O)NHC2H5 |
| 6-2-218 | CH | N | N | CNHC(═O)NHCH2CCH |
| 6-2-219 | CH | N | N | CNC(═S)NHC2H5 |
| 6-2-220 | CH | N | N | CNHC(═S)C2H5 |
| 6-2-221 | CH | CH | N | CNH2 |
| 6-2-222 | CH | CH | N | CNH(C═O)CH3 |
| 6-2-223 | CH | CH | N | CNH(C═O)C2H5 |
| 6-2-224 | CH | CH | N | CNH(C═O)CH2CF3 |
| 6-2-225 | CH | CH | N | CNH(C═O)Pr-c |
| 6-2-226 | CH | CH | N | CNH(C═O)NHC2H5 |
| 6-2-227 | N | CH | CH | CNH2 |
| 6-2-228 | N | CH | CH | CNH(C═O)C2H5 |
| 6-2-229 | N | CH | CH | CNH(C═O)NHC2H5 |
| 6-2-230 | CH | N | CH | CNH2 |
| 6-2-231 | CH | N | CH | CNH(C═O)Pr-c |
| 6-2-232 | CH | N | CH | CNH(C═O)CH2S(═O)2Me |
| 6-2-233 | N | N | CH | CNH2 |
| 6-2-234 | N | N | CH | CNH(C═O)CH2CF3 |
| 6-2-235 | N | N | CH | CNH(C═O)CH2SMe |
| 6-2-236 | N | CH | N | CNH2 |
| 6-2-237 | N | CH | N | CNH(C═O)CH3 |
| 6-2-238 | N | CH | N | CNH(C═O)CH2S(═O)Me |
| 6-2-239 | CH | NCH3 | N | CNH2 |
| 6-2-240 | CH | NCH3 | N | CNH(C═O)C2H5 |
| 6-2-241 | CH | NCH3 | N | CNH(C═O)NHC2H5 |
| 6-2-242 | CH2 | O | N | CNH2 |
| 6-2-243 | CH2 | O | N | CNH(C═O)Pr-c |
| 6-2-244 | CH2 | O | N | CNH(C═O)CH2S(═O)2Me |
| 6-2-245 | CH2 | CH2 | CHCH3 | CNH2 |
| 6-2-246 | CH2 | CH2 | CHCH3 | CNH(C═O)CH3 |
| 6-2-247 | CH2 | CH2 | CHCH3 | CNH(C═O)C2H5 |
| 6-2-248 | CH2 | CH2 | CHCH3 | CNH(C═O)CH2CF3 |
| 6-2-249 | CH2 | CH2 | CHCH3 | CNH(C═O)Pr-c |
| 6-2-250 | CH2 | CH2 | CHCH3 | CNH(C═O)CH2Pr-c |
| 6-2-251 | CH2 | CH2 | CHCH3 | CNH(C═O)CH2SMe |
| 6-2-252 | CH2 | CH2 | CHCH3 | CNH(C═O)CH2S(═O)Me |
| 6-2-253 | CH2 | CH2 | CHCH3 | CNH(C═O)CH2S(═O)2Me |
| 6-2-254 | CH2 | CH2 | CHCH3 | CNHC(═O)NHCH3 |
| 6-2-255 | CH2 | CH2 | CHCH3 | CNH(C═O)NHC2H5 |
| 6-2-256 | CH2 | CH2 | CHCH3 | CNHC(═O)NHCH2CCH |
| 6-2-257 | CH2 | CH2 | CHCH3 | CNHCO2Bu-t |
| 6-2-258 | CH2 | CH2 | CHCH3 | CNHCO2CH3 |
| 6-2-259 | CH2 | CH2 | CHCH3 | CNHCO2Ph |
| 6-2-260 | CH2 | CH2 | CHCH3 | CNC(═S)NHC2H5 |
| 6-2-261 | CH2 | CH2 | CHCH3 | CHNH(C═S)C2H5 |
| 6-2-262 | CH2 | CH2 | C(CH3)2 | CNH2 |
| 6-2-263 | CH2 | CH2 | C(CH3)2 | CNH(C═O)CH3 |
| 6-2-264 | CH2 | CH2 | C(CH3)2 | CNH(C═O)NHC2H5 |
| 6-2-265 | CH2 | CH2 | C(CH3)2 | CNHCO2Bu-t |
| 6-2-266 | CH2 | CH2 | CHF | CNH2 |
| 6-2-267 | CH2 | CH2 | CHF | CNH(C═O)CH3 |
| 6-2-268 | CH2 | CH2 | CHF | CNH(C═O)NHC2H5 |
| 6-2-269 | CH2 | CH2 | CHF | CNHCO2Bu-t |
| 6-2-270 | CH2 | CH2 | CHCl | CNH2 |
| 6-2-271 | CH2 | CH2 | CHCl | CNH(C═O)CH3 |
| 6-2-272 | CH2 | CH2 | CHCl | CNH(C═O)NHC2H5 |
| 6-2-273 | CH2 | CH2 | CHCl | CNHCO2Bu-t |

TABLE 7

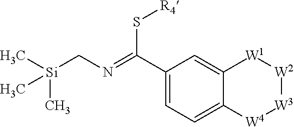

| Example | R₄′ | W¹ | W² | W³ | W⁴ |
|---|---|---|---|---|---|
| 7-1 | Me | — | CH2 | O | C═O |
| 7-2 | Me | — | CH2 | O | CH2 |
| 7-3 | Me | — | CH2 | CH2 | O |
| 7-4 | Me | — | O | CH2 | O |
| 7-5 | Me | — | O | CF2 | O |
| 7-6 | Me | — | CH | CH | O |
| 7-7 | Me | — | CH2 | CH2 | S |
| 7-8 | Me | — | CH2 | S | CH2 |
| 7-9 | Me | — | CH | CH | S |
| 7-10 | Me | — | CH2 | CH2 | NH |
| 7-11 | Me | — | CH2 | NH | CH2 |
| 7-12 | Me | — | NH | CH2 | CH2 |
| 7-13 | Me | — | CH | CH | NH |
| 7-14 | Me | — | CH | N | NH |
| 7-15 | Me | — | C═O | O | CH2 |
| 7-16 | Me | — | CH2 | S | C═O |
| 7-17 | Me | — | N | CH | O |
| 7-18 | Me | — | N | CH | S |
| 7-19 | Me | — | N | CH | NH |
| 7-20 | Me | — | O | CH | N |
| 7-21 | Me | — | S | CH | N |
| 7-22 | Me | — | CH | N | O |
| 7-23 | Me | — | CH | N | S |
| 7-24 | Me | — | CH2 | CH2 | C═O |
| 7-25 | Me | — | CH | CH | C═O |
| 7-26 | Me | — | CH2 | C═O | CH2 |
| 7-27 | Me | — | C═O | CH2 | CH2 |
| 7-28 | Me | — | CH2 | C═O | NH |
| 7-29 | Me | — | NH | C═O | CH2 |
| 7-30 | Me | — | CH2 | C═O | N(C═O)CH3 |
| 7-31 | Me | — | CH2 | C═O | N(C═O)CH2CF3 |
| 7-32 | Me | — | CH2 | CH2 | N(C═O)CH3 |
| 7-33 | Me | — | CH2 | CH2 | N(C═O)C2H5 |
| 7-34 | Me | — | CH2 | CH2 | N(C═O)CH2CF3 |
| 7-35 | Me | — | CH2 | CH2 | N(C═O)NCH3 |
| 7-36 | Me | — | CH2 | CH2 | N(C═O)NC2H5 |
| 7-37 | Me | — | CH2 | CH2 | N(C═O)NCH2CCH |
| 7-38 | Me | — | CH2 | N(C═O)CH3 | CH2 |
| 7-39 | Me | — | CH2 | N(C═O)C2H5 | CH2 |
| 7-40 | Me | — | CH2 | N(C═O)CH2CF3 | CH2 |
| 7-41 | Me | — | CH2 | N(C═O)Pr-c | CH2 |
| 7-42 | Me | — | CH2 | N(C═O)NCH3 | CH2 |
| 7-43 | Me | — | CH2 | N(C═O)NC2H5 | CH2 |
| 7-44 | Me | — | CH2 | N(C═O)NCH2CCH | CH2 |
| 7-45 | Me | — | CH2 | O | C═NH |
| 7-46 | Me | — | CH2 | S | C═NH |
| 7-47 | Me | — | CH2 | N | C═NH |
| 7-48 | Me | — | CH | CH | N(C═O)CH3 |
| 7-49 | Me | — | CH | CH | N(C═O)C2H5 |
| 7-50 | Me | — | CH | CH | N(C═O)NC2H5 |
| 7-51 | Me | — | CH | CH | N(C═O)NCH2CCH |
| 7-52 | Me | — | CH2 | CH2 | C═NOH |
| 7-53 | Me | — | CH2 | CH2 | C═NOCH3 |
| 7-54 | Me | — | CH2 | CH2 | C═NOCH2CF3 |
| 7-55 | Me | — | CH2 | CH2 | CHNH2 |
| 7-56 | Me | — | CH2 | CH2 | CHNH(C═O)CH3 |
| 7-57 | Me | — | CH2 | CH2 | CHNH(C═O)C2H5 |
| 7-58 | Me | — | CH2 | CH2 | CHNH(C═O)CH2CF3 |
| 7-59 | Me | — | CH2 | CH2 | CHNH(C═O)Pr-c |
| 7-60 | Me | — | CH2 | CH2 | CHNH(C═O)CH2Pr-c |
| 7-61 | Me | — | CH2 | CH2 | CHNH(C═O)CH2SMe |
| 7-62 | Me | — | CH2 | CH2 | CHNH(C═O)CH2S(═O)Me |
| 7-63 | Me | — | CH2 | CH2 | CHNH(C═O)CH2S(═O)2Me |
| 7-64 | Me | — | CH2 | CH2 | CHNH(C═O)NHCH3 |
| 7-65 | Me | — | CH2 | CH2 | CHNH(C═O)NHC2H5 |
| 7-66 | Me | — | CH2 | CH2 | CHNH(C═O)NHCH2CCH |
| 7-67 | Me | — | CH2 | CH2 | CHNHCO2C4H9-tert |
| 7-68 | Me | — | CH2 | CH2 | CHNHCO2CH3 |
| 7-69 | Me | — | CH2 | CH2 | CHNHCO2Ph |
| 7-70 | Me | — | CH2 | CH2 | CHO(C═O)CH3 |
| 7-71 | Me | — | CH2 | CH2 | CHN3 |
| 7-72 | Me | — | CH2 | CH2 | CHNH(C═S)NHC2H5 |
| 7-73 | Me | — | CH2 | CH2 | CHNH(C═S)C2H5 |
| 7-74 | Me | — | CH2 | CHNH2 | CH2 |
| 7-75 | Me | — | CH2 | CHNH(C═O)C2H5 | CH2 |
| 7-76 | Me | — | CH2 | CHNHCO2C4H9-tert | CH2 |
| 7-77 | Me | — | CH2 | C═O | CH2 |
| 7-78 | Me | — | CH2 | C═NOH | CH2 |
| 7-79 | Me | — | O | N | CNH2 |
| 7-80 | Me | — | O | N | CNHCO2C4H9-tert |
| 7-81 | Me | — | NCH3 | N | CNH2 |
| 7-82 | Me | — | NCH3 | N | CNHCO2C4H9-tert |
| 7-83 | Me | — | SO2 | N | CNH2 |
| 7-84 | Me | — | SO2 | N | CNHCO2C4H9-tert |
| 7-85 | Me | — | CH | CH | CNH2 |
| 7-86 | Me | — | CH | CH | CNH(C═O)CH3 |
| 7-87 | Me | — | CH | CH | CNH(C═O)C2H5 |
| 7-88 | Me | — | CH | CH | CNH(C═O)CH2CF3 |
| 7-89 | Me | — | CH | CH | CNH(C═O)Pr-c |
| 7-90 | Me | — | CH | CH | CNH(C═O)CH2SMe |
| 7-91 | Me | — | CH | CH | CNH(C═O)CH2S(═O)Me |
| 7-92 | Me | — | CH | CH | CNH(C═O)CH2S(═O)2Me |
| 7-93 | Me | — | CH | CH | CNH(C═O)NHC2H5 |
| 7-94 | Me | — | CH | CH | CNHCO2C4H9-tert |
| 7-95 | Me | — | CH | CH | CO(C═O)CH3 |
| 7-96 | Me | — | CH | CH | CHN3 |
| 7-97 | Me | — | CH2 | CH2 | NNH2 |
| 7-98 | Me | — | CH2 | CH2 | NNH(C═O)CH3 |
| 7-99 | Me | — | CH2 | CH2 | NNH(C═O)C2H5 |
| 7-100 | Me | — | CH2 | CH2 | NNH(C═O)CH2CF3 |
| 7-101 | Me | — | CH2 | CH2 | NNH(C═O)Pr-c |
| 7-102 | Me | — | CH2 | CH2 | NNH(C═O)CH2SMe |
| 7-103 | Me | — | CH2 | CH2 | NNH(C═O)CH2S(═O)Me |
| 7-104 | Me | — | CH2 | CH2 | NNH(C═O)CH2S(═O)2Me |
| 7-105 | Me | — | CH2 | CH2 | NNH(C═O)NHC2H5 |
| 7-106 | Me | — | CH2 | CH2 | NNHCO2Bu-t |
| 7-107 | Me | — | CH2 | CHCH3 | CHNHCO2C4H9-tert |
| 7-108 | Me | — | CH2 | C(CH3)2 | CHNHCO2C4H9-tert |
| 7-109 | Me | — | CH2 | CHCl | CHNHCO2C4H9-tert |
| 7-110 | Me | — | CH2 | CHF | CHNHCO2C4H9-tert |
| 7-111 | Me | CH2 | CH2 | O | C═O |
| 7-112 | Me | CH2 | CH2 | O | CH2 |
| 7-113 | Me | CH2 | O | CH2 | CH2 |
| 7-114 | Me | CH2 | CH2 | CH2 | O |
| 7-115 | Me | CH2 | CH | CH | N |
| 7-116 | Me | CH2 | CH | N | CH |
| 7-117 | Me | CH2 | N | CH | CH |
| 7-118 | Me | CH2 | CH | CBr | N |
| 7-119 | Me | CH2 | CH | CCl | N |
| 7-120 | Me | CH2 | CH | CCN | N |
| 7-121 | Me | CH2 | CH2 | CH2 | NH |
| 7-122 | Me | CH2 | CH2 | CH2 | N(C═O)CH3 |
| 7-123 | Me | CH2 | CH2 | CH2 | N(C═O)NC2H5 |
| 7-124 | Me | CH2 | CH2 | NH | CH2 |
| 7-125 | Me | CH2 | CH2 | N(C═O)CH3 | CH2 |
| 7-126 | Me | CH2 | CH2 | N(C═O)NC2H5 | CH2 |
| 7-127 | Me | CH2 | CH2 | C═O | N |
| 7-128 | Me | CH2 | C═O | N | CH2 |
| 7-129 | Me | CH2 | N | C═O | CH2 |
| 7-130 | Me | CH2 | NH | NH | C═O |
| 7-131 | Me | CH2 | NCH3 | NCH3 | C═O |
| 7-132 | Me | CH2 | NCH3 | NH | C═O |
| 7-133 | Me | CH | N | NH | C═O |
| 7-134 | Me | CH2 | O | N | C═O |
| 7-135 | Me | CH2 | N | O | C═O |
| 7-136 | Me | CH | N | O | C═O |
| 7-137 | Me | CH | N | CH | N |
| 7-138 | Me | CH | N | N(C═O)CH3 | C═O |
| 7-139 | Me | CH | N | N(C═O)C2H5 | C═O |
| 7-140 | Me | CH | N | N(C═O)Pr-c | C═O |
| 7-141 | Me | CH | N | N(C═O)CH2CF3 | C═O |
| 7-142 | Me | CH | N | NC(═O)NHC2H5 | C═O |
| 7-143 | Me | CH2 | O | N(C═O)CH3 | C═O |
| 7-144 | Me | CH2 | NCH3 | N(C═O)CH3 | C═O |
| 7-145 | Me | CH2 | CH2 | CH2 | N(C═O)C2H5 |
| 7-146 | Me | CH2 | CH2 | CH2 | N(C═O)CH2CF3 |
| 7-147 | Me | CH2 | CH2 | CH2 | N(C═O)NCH3 |
| 7-148 | Me | CH2 | CH2 | CH2 | N(C═O)NCH2CCH |
| 7-149 | Me | CH2 | CH2 | N(C═O)CH3 | CH2 |
| 7-150 | Me | CH2 | CH2 | N(C═O)C2H5 | CH2 |
| 7-151 | Me | CH2 | CH2 | N(C═O)CH2CF3 | CH2 |
| 7-152 | Me | CH2 | CH2 | N(C═O)Pr-c | CH2 |
| 7-153 | Me | CH2 | CH2 | N(C═O)NCH3 | CH2 |
| 7-154 | Me | CH2 | CH2 | N(C═O)NC2H5 | CH2 |
| 7-155 | Me | CH2 | CH2 | N(C═O)NCH2CCH | CH2 |
| 7-156 | Me | CH2 | CH2 | CH2 | C═O |
| 7-157 | Me | CH2 | CH2 | CH2 | C═NOH |
| 7-158 | Me | CH2 | CH2 | CH2 | C═NOCH3 |
| 7-159 | Me | CH2 | CH2 | CH2 | C═NOCH2CF3 |
| 7-160 | Me | CH2 | CH2 | CH2 | CHNH2 |
| 7-161 | Me | CH2 | CH2 | CH2 | CHNH(C═O)CH3 |
| 7-162 | Me | CH2 | CH2 | CH2 | CHNH(C═O)C2H5 |
| 7-163 | Me | CH2 | CH2 | CH2 | CHNH(C═O)CH2CF3 |
| 7-164 | Me | CH2 | CH2 | CH2 | CHNH(C═O)Pr-c |
| 7-165 | Me | CH2 | CH2 | CH2 | CHNH(C═O)CH2Pr-c |
| 7-166 | Me | CH2 | CH2 | CH2 | CHNH(C═O)CH2SMe |
| 7-167 | Me | CH2 | CH2 | CH2 | CHNH(C═O)CH2S(═O)Me |
| 7-168 | Me | CH2 | CH2 | CH2 | CHNH(C═O)CH2S(═O)2Me |
| 7-169 | Me | CH2 | CH2 | CH2 | CHNH(C═O)NHCH3 |
| 7-170 | Me | CH2 | CH2 | CH2 | CHNH(C═O)NHC2H5 |
| 7-171 | Me | CH2 | CH2 | CH2 | CHNH(C═O)NHCH2CCH |
| 7-172 | Me | CH2 | CH2 | CH2 | CHNHCO2C4H9-tert |
| 7-173 | Me | CH2 | CH2 | CH2 | CHNHCO2CH3 |
| 7-174 | Me | CH2 | CH2 | CH2 | CHNHCO2Ph |
| 7-175 | Me | CH2 | CH2 | CH2 | CHO(C═O)CH3 |
| 7-176 | Me | CH2 | CH2 | CH2 | CHN3 |
| 7-177 | Me | CH2 | CH2 | CH2 | CHNH(C═S)NHC2H5 |
| 7-178 | Me | CH2 | CH2 | CH2 | CHNH(C═S)C2H5 |
| 7-179 | Me | CH2 | CH2 | CHNH2 | CH2 |
| 7-180 | Me | CH2 | CH2 | CHNH(C═O)C2H5 | CH2 |
| 7-181 | Me | CH2 | CH2 | CHNHCO2C4H9-tert | CH2 |
| 7-182 | Me | CH2 | CH2 | C═O | CH2 |
| 7-183 | Me | CH2 | CH2 | C═NOH | CH2 |
| 7-184 | Me | CH | CH | CH | CNH2 |
| 7-185 | Me | CH | CH | CH | CNH(C═O)CH3 |
| 7-186 | Me | CH | CH | CH | CNH(C═O)C2H5 |
| 7-187 | Me | CH | CH | CH | CNH(C═O)CH2CF3 |
| 7-188 | Me | CH | CH | CH | CNH(C═O)Pr-c |
| 7-189 | Me | CH | CH | CH | CNH(C═O)CH2Pr-c |
| 7-190 | Me | CH | CH | CH | CNH(C═O)CH2SMe |
| 7-191 | Me | CH | CH | CH | CNH(C═O)CH2S(═O)Me |
| 7-192 | Me | CH | CH | CH | CNH(C═O)CH2S(═O)2Me |
| 7-193 | Me | CH | CH | CH | CNH(C═O)NHCH3 |
| 7-194 | Me | CH | CH | CH | CNH(C═O)NHC2H5 |
| 7-195 | Me | CH | CH | CH | CNH(C═O)NHCH2CCH |
| 7-196 | Me | CH | CH | CH | CNHCO2C4H9-tert |
| 7-197 | Me | CH | CH | CH | CNHCO2CH3 |
| 7-198 | Me | CH | CH | CH | CNHCO2Ph |
| 7-199 | Me | CH | CH | CH | CHN(C═S)NHC2H5 |
| 7-200 | Me | CH | CH | CH | CHNH(C═S)C2H5 |
| 7-201 | Me | CH | CH | CNH2 | CH |
| 7-202 | Me | CH | CH | CNH(C═O)CH3 | CH |
| 7-203 | Me | CH | CH | CNH(C═O)C2H5 | CH |
| 7-204 | Me | CH | CH | CNH(C═O)Pr-c | CH |
| 7-205 | Me | CH | CH | CNH(C═O)NHC2H5 | CH |
| 7-206 | Me | CH | CH | CNHCO2C4H9-tert | CH |
| 7-207 | Me | CH | N | N | CCI |
| 7-208 | Me | CH | N | N | CNH2 |
| 7-209 | Me | CH | N | N | CNH(C═O)CH3 |
| 7-210 | Me | CH | N | N | CNH(C═O)C2H5 |
| 7-211 | Me | CH | N | N | CNH(C═O)CH2CF3 |
| 7-212 | Me | CH | N | N | CNH(C═O)Pr-c |
| 7-213 | Me | CH | N | N | CNH(C═O)CH2Pr-c |
| 7-214 | Me | CH | N | N | CNH(C═O)CH2SMe |
| 7-215 | Me | CH | N | N | CNH(C═O)CH2S(═O)Me |
| 7-216 | Me | CH | N | N | CNH(C═O)CH2S(═O)2Me |
| 7-217 | Me | CH | N | N | CNH(C═O)NHCH3 |
| 7-218 | Me | CH | N | N | CNH(C═O)NHC2H5 |
| 7-219 | Me | CH | N | N | CNH(C═O)NHCH2CCH |
| 7-220 | Me | CH | N | N | CHN(C═S)NHC2H5 |
| 7-221 | Me | CH | N | N | CNH(C═S)C2H5 |
| 7-222 | Me | CH | CH | N | CNH2 |
| 7-223 | Me | CH | CH | N | CNH(C═O)CH3 |
| 7-224 | Me | CH | CH | N | CNH(C═O)C2H5 |
| 7-225 | Me | CH | CH | N | CNH(C═O)CH2CF3 |
| 7-226 | Me | CH | CH | N | CNH(C═O)Pr-c |
| 7-227 | Me | CH | CH | N | CNH(C═O)NHC2H5 |
| 7-228 | Me | N | CH | CH | CNH2 |
| 7-229 | Me | N | CH | CH | CNH(C═O)C2H5 |
| 7-230 | Me | N | CH | CH | CNH(C═O)NHC2H5 |
| 7-231 | Me | CH | N | CH | CNH2 |
| 7-232 | Me | CH | N | CH | CNH(C═O)Pr-c |
| 7-233 | Me | CH | N | CH | CNH(C═O)CH2S(═O)2Me |
| 7-234 | Me | N | N | CH | CNH2 |
| 7-235 | Me | N | N | CH | CNH(C═O)CH2CF3 |
| 7-236 | Me | N | N | CH | CNH(C═O)CH2SMe |
| 7-237 | Me | N | CH | N | CNH2 |
| 7-238 | Me | N | CH | N | CNH(C═O)CH3 |
| 7-239 | Me | N | CH | N | CNH(C═O)CH2S(═O)Me |
| 7-240 | Me | CH | NCH3 | N | CNH2 |
| 7-241 | Me | CH | NCH3 | N | CNH(C═O)C2H5 |
| 7-242 | Me | CH | NCH3 | N | CNH(C═O)NHC2H5 |
| 7-243 | Me | CH2 | O | N | CNH2 |
| 7-244 | Me | CH2 | O | N | CNH(C═O)Pr-c |
| 7-245 | Me | CH2 | O | N | CNH(C═O)CH2S(═O)2Me |
| 7-246 | Me | CH2 | CH2 | CHCH3 | CHNH2 |
| 7-247 | Me | CH2 | CH2 | CHCH3 | CHNH(C═O)CH3 |
| 7-248 | Me | CH2 | CH2 | CHCH3 | CHNH(C═O)C2H5 |
| 7-249 | Me | CH2 | CH2 | CHCH3 | CHNH(C═O)CH2CF3 |
| 7-250 | Me | CH2 | CH2 | CHCH3 | CHNH(C═O)Pr-c |
| 7-251 | Me | CH2 | CH2 | CHCH3 | CHNH(C═O)CH2Pr-c |
| 7-252 | Me | CH2 | CH2 | CHCH3 | CHNH(C═O)CH2SMe |
| 7-253 | Me | CH2 | CH2 | CHCH3 | CHNH(C═O)CH2S(═O)Me |
| 7-254 | Me | CH2 | CH2 | CHCH3 | CHNH(C═O)CH2S(═O)2Me |
| 7-255 | Me | CH2 | CH2 | CHCH3 | CHNH(C═O)NHCH3 |
| 7-256 | Me | CH2 | CH2 | CHCH3 | CHNH(C═O)NHC2H5 |
| 7-257 | Me | CH2 | CH2 | CHCH3 | CHNH(C═O)NHCH2CCH |
| 7-258 | Me | CH2 | CH2 | CHCH3 | CHNHCO2C4H9-tert |
| 7-259 | Me | CH2 | CH2 | CHCH3 | CHNHCO2CH3 |
| 7-260 | Me | CH2 | CH2 | CHCH3 | CHNHCO2Ph |
| 7-261 | Me | CH2 | CH2 | CHCH3 | CHN(C═S)NHC2H5 |
| 7-262 | Me | CH2 | CH2 | CHCH3 | CHHNH(C═S)C2H5 |
| 7-263 | Me | CH2 | CH2 | C(CH3)2 | CNH2 |
| 7-264 | Me | CH2 | CH2 | C(CH3)2 | CNH(C═O)CH3 |
| 7-265 | Me | CH2 | CH2 | C(CH3)2 | CNH(C═O)NHC2H5 |
| 7-266 | Me | CH2 | CH2 | C(CH3)2 | CNHCO2C4H9-tert |
| 7-267 | Me | CH2 | CH2 | CHF | CNH2 |
| 7-268 | Me | CH2 | CH2 | CHF | CNH(C═O)CH3 |
| 7-269 | Me | CH2 | CH2 | CHF | CNH(C═O)NHC2H5 |
| 7-270 | Me | CH2 | CH2 | CHF | CNHCO2C4H9-tert |
| 7-271 | Me | CH2 | CH2 | CHCl | CNH2 |
| 7-272 | Me | CH2 | CH2 | CHCl | CNH(C═O)CH3 |
| 7-273 | Me | CH2 | CH2 | CHCl | CNH(C═O)NHC2H5 |
| 7-274 | Me | CH2 | CH2 | CHCl | CNHCO2C4H9-tert |
| 7-275 | H | — | CH2 | O | C═O |
| 7-276 | H | — | CH2 | O | CH2 |
| 7-277 | H | — | CH2 | CH2 | O |
| 7-278 | H | — | O | CH2 | O |
| 7-279 | H | — | O | CF2 | O |
| 7-280 | H | — | CH | CH | O |
| 7-281 | H | — | CH2 | CH2 | S |
| 7-282 | H | — | CH2 | S | CH2 |
| 7-283 | H | — | CH | CH | S |
| 7-284 | H | — | CH2 | CH2 | NH |
| 7-285 | H | — | CH2 | NH | CH2 |
| 7-286 | H | — | NH | CH2 | CH2 |
| 7-287 | H | — | CH | CH | NH |
| 7-288 | H | — | CH | N | NH |
| 7-289 | H | — | C═O | O | CH2 |
| 7-290 | H | — | CH2 | S | C═O |
| 7-291 | H | — | N | CH | O |
| 7-292 | H | — | N | CH | S |
| 7-293 | H | — | N | CH | NH |
| 7-294 | H | — | O | CH | N |
| 7-295 | H | — | S | CH | N |
| 7-296 | H | — | CH | N | O |
| 7-297 | H | — | CH | N | S |
| 7-298 | H | — | CH2 | CH2 | C═O |
| 7-299 | H | — | CH | CH | C═O |
| 7-300 | H | — | CH2 | C═O | CH2 |
| 7-301 | H | — | C═O | CH2 | CH2 |
| 7-302 | H | — | CH2 | C═O | NH |
| 7-303 | H | — | NH | C═O | CH2 |
| 7-304 | H | — | CH2 | C═O | N(C═O)CH3 |
| 7-305 | H | — | CH2 | C═O | N(C═O)CH2CF3 |
| 7-306 | H | — | CH2 | CH2 | N(C═O)CH3 |
| 7-307 | H | — | CH2 | CH2 | N(C═O)C2H5 |
| 7-308 | H | — | CH2 | CH2 | N(C═O)CH2CF3 |
| 7-309 | H | — | CH2 | CH2 | N(C═O)NCH3 |
| 7-310 | H | — | CH2 | CH2 | N(C═O)NC2H5 |
| 7-311 | H | — | CH2 | CH2 | N(C═O)NCH2CCH |
| 7-312 | H | — | CH2 | N(C═O)CH3 | CH2 |
| 7-313 | H | — | CH2 | N(C═O)C2H5 | CH2 |
| 7-314 | H | — | CH2 | N(C═O)CH2CF3 | CH2 |
| 7-315 | H | — | CH2 | N(C═O)Pr-c | CH2 |
| 7-316 | H | — | CH2 | N(C═O)NCH3 | CH2 |
| 7-317 | H | — | CH2 | N(C═O)NC2H5 | CH2 |
| 7-318 | H | — | CH2 | N(C═O)NCH2CCH | CH2 |
| 7-319 | H | — | CH2 | 0 | C═NH |
| 7-320 | H | — | CH2 | S | C═NH |
| 7-321 | H | — | CH2 | N | C═NH |
| 7-322 | H | — | CH | CH | N(C═O)CH3 |
| 7-323 | H | — | CH | CH | N(C═O)C2H5 |
| 7-324 | H | — | CH | CH | N(C═O)NC2H5 |
| 7-325 | H | — | CH | CH | N(C═O)NCH2CCH |
| 7-326 | H | — | CH2 | CH2 | C═NOH |
| 7-327 | H | — | CH2 | CH2 | C═NOCH3 |
| 7-328 | H | — | CH2 | CH2 | C═NOCH2CF3 |
| 7-329 | H | — | CH2 | CH2 | CHNH2 |
| 7-330 | H | — | CH2 | CH2 | CHNH(C═O)CH3 |
| 7-331 | H | — | CH2 | CH2 | CHNH(C═O)C2 H5 |
| 7-332 | H | — | CH2 | CH2 | CHNH(C═O)CH2CF3 |
| 7-333 | H | — | CH2 | CH2 | CHNH(C═O)Pr-c |
| 7-334 | H | — | CH2 | CH2 | CHNH(C═O)CH2Pr-c |
| 7-335 | H | — | CH2 | CH2 | CHNH(C═O)CH2SMe |
| 7-336 | H | — | CH2 | CH2 | CHNH(C═O)CH2S(═O)Me |
| 7-337 | H | — | CH2 | CH2 | CHNH(C═O)CH2S(═O)2Me |
| 7-338 | H | — | CH2 | CH2 | CHNH(C═O)NHCH3 |
| 7-339 | H | — | CH2 | CH2 | CHNH(C═O)NHC2H5 |
| 7-340 | H | — | CH2 | CH2 | CHNH(C═O)NHCH2CCH |
| 7-341 | H | — | CH2 | CH2 | CHNHCO2C4H9-tert |
| 7-342 | H | — | CH2 | CH2 | CHNHCO2CH3 |
| 7-343 | H | — | CH2 | CH2 | CHNHCO2Ph |
| 7-344 | H | — | CH2 | CH2 | CHO(C═O)CH3 |
| 7-345 | H | — | CH2 | CH2 | CHN3 |
| 7-346 | H | — | CH2 | CH2 | CHNH(C═S)NHC2H5 |
| 7-347 | H | — | CH2 | CH2 | CHNH(C═S)C2H5 |
| 7-348 | H | — | CH2 | CHNH2 | CH2 |
| 7-349 | H | — | CH2 | CHNH(C═O)C2H5 | CH2 |
| 7-350 | H | — | CH2 | CHNHCO2C4H9-tert | CH2 |
| 7-351 | H | — | CH2 | C═O | CH2 |
| 7-352 | H | — | CH2 | C═NOH | CH2 |
| 7-353 | H | — | O | N | CNH2 |
| 7-354 | H | — | O | N | CNHCO2C4H9-tert |
| 7-355 | H | — | NCH3 | N | CNH2 |
| 7-356 | H | — | NCH3 | N | CNHCO2C4H9-tert |
| 7-357 | H | — | SO2 | N | CNH2 |
| 7-358 | H | — | SO2 | N | CNHCO2C4H9-tert |
| 7-359 | H | — | CH | CH | CNH2 |
| 7-360 | H | — | CH | CH | CNH(C═O)CH3 |
| 7-361 | H | — | CH | CH | CNH(C═O)C2H5 |
| 7-362 | H | — | CH | CH | CNH(C═O)CH2CF3 |
| 7-363 | H | — | CH | CH | CNH(C═O)Pr-c |
| 7-364 | H | — | CH | CH | CNH(C═O)CH2SMe |
| 7-365 | H | — | CH | CH | CNH(C═O)CH2S(═O)Me |
| 7-366 | H | — | CH | CH | CNH(C═O)CH2S(═O)2Me |
| 7-367 | H | — | CH | CH | CNH(C═O)NHC2H5 |
| 7-368 | H | — | CH | CH | CNHCO2C4H9-tert |
| 7-369 | H | — | CH | CH | CO(C═O)CH3 |
| 7-370 | H | — | CH | CH | CHN3 |
| 7-371 | H | — | CH2 | CH2 | NNH2 |
| 7-372 | H | — | CH2 | CH2 | NNH(C═O)CH3 |
| 7-373 | H | — | CH2 | CH2 | NNH(C═O)C2H5 |
| 7-374 | H | — | CH2 | CH2 | NNH(C═O)CH2CF3 |
| 7-375 | H | — | CH2 | CH2 | NNH(C═O)Pr-c |
| 7-376 | H | — | CH2 | CH2 | NNH(C═O)CH2SMe |
| 7-377 | H | — | CH2 | CH2 | NNH(C═O)CH2S(═O)Me |
| 7-378 | H | — | CH2 | CH2 | NNH(C═O)CH2S(═O)2Me |
| 7-379 | H | — | CH2 | CH2 | NNH(C═O)NHC2H5 |
| 7-380 | H | — | CH2 | CH2 | NNHCO2Bu-t |
| 7-381 | H | — | CH2 | CHCH3 | CHNHCO2C4H9-tert |
| 7-382 | H | — | CH2 | C(CH3)2 | CHNHCO2C4H9-tert |
| 7-383 | H | — | CH2 | CHCI | CHNHCO2C4H9-tert |
| 7-384 | H | — | CH2 | CHF | CHNHCO2C4H9-tert |
| 7-385 | H | CH2 | CH2 | O | C═O |
| 7-386 | H | CH2 | CH2 | O | CH2 |
| 7-387 | H | CH2 | O | CH2 | CH2 |
| 7-388 | H | CH2 | CH2 | CH2 | O |
| 7-389 | H | CH2 | CH | CH | N |
| 7-390 | H | CH2 | CH | N | CH |
| 7-391 | H | CH2 | N | CH | CH |
| 7-392 | H | CH2 | CH | CBr | N |
| 7-393 | H | CH2 | CH | CCl | N |
| 7-394 | H | CH2 | CH | CCN | N |
| 7-395 | H | CH2 | CH2 | CH2 | NH |
| 7-396 | H | CH2 | CH2 | CH2 | N(C═O)CH3 |
| 7-397 | H | CH2 | CH2 | CH2 | N(C═O)NC2H5 |
| 7-398 | H | CH2 | CH2 | NH | CH2 |
| 7-399 | H | CH2 | CH2 | N(C═O)CH3 | CH2 |
| 7-400 | H | CH2 | CH2 | N(C═O)NC2H5 | CH2 |
| 7-401 | H | CH2 | CH2 | C═O | N |
| 7-402 | H | CH2 | C═O | N | CH2 |
| 7-403 | H | CH2 | N | C═O | CH2 |
| 7-404 | H | CH2 | NH | NH | C═O |
| 7-405 | H | CH2 | NCH3 | NCH3 | C═O |
| 7-406 | H | CH2 | NCH3 | NH | C═O |
| 7-407 | H | CH | N | NH | C═O |
| 7-408 | H | CH2 | O | N | C═O |
| 7-409 | H | CH2 | N | O | C═O |
| 7-410 | H | CH | N | O | C═O |
| 7-411 | H | CH | N | CH | N |
| 7-412 | H | CH | N | N(C═O)CH3 | C═O |
| 7-413 | H | CH | N | N(C═O)C2H5 | C═O |
| 7-414 | H | CH | N | N(C═O)Pr-c | C═O |
| 7-415 | H | CH | N | N(C═O)CH2CF3 | C═O |
| 7-416 | H | CH | N | NC(═O)NHC2H5 | C═O |
| 7-417 | H | CH2 | O | N(C═O)CH3 | C═O |
| 7-418 | H | CH2 | NCH3 | N(C═O)CH3 | C═O |
| 7-419 | H | CH2 | CH2 | CH2 | N(C═O)C2H5 |
| 7-420 | H | CH2 | CH2 | CH2 | N(C═O)CH2CF3 |
| 7-421 | H | CH2 | CH2 | CH2 | N(C═O)NCH3 |
| 7-422 | H | CH2 | CH2 | CH2 | N(C═O)NCH2CCH |
| 7-423 | H | CH2 | CH2 | N(C═O)CH3 | CH2 |
| 7-424 | H | CH2 | CH2 | N(C═O)C2H5 | CH2 |
| 7-425 | H | CH2 | CH2 | N(C═O)CH2CF3 | CH2 |
| 7-426 | H | CH2 | CH2 | N(C═O)Pr-c | CH2 |
| 7-427 | H | CH2 | CH2 | N(C═O)NCH3 | CH2 |
| 7-428 | H | CH2 | CH2 | N(C═O)NC2H5 | CH2 |
| 7-429 | H | CH2 | CH2 | N(C═O)NCH2CCH | CH2 |
| 7-430 | H | CH2 | CH2 | CH2 | C═O |
| 7-431 | H | CH2 | CH2 | CH2 | C═NOH |
| 7-432 | H | CH2 | CH2 | CH2 | C═NOCH3 |
| 7-433 | H | CH2 | CH2 | CH2 | C═NOCH2CF3 |
| 7-434 | H | CH2 | CH2 | CH2 | CHNH2 |
| 7-435 | H | CH2 | CH2 | CH2 | CHNH(C═O)CH3 |
| 7-436 | H | CH2 | CH2 | CH2 | CHNH(C═O)C2H5 |
| 7-437 | H | CH2 | CH2 | CH2 | CHNH(C═O)CH2CF3 |
| 7-438 | H | CH2 | CH2 | CH2 | CHNH(C═O)Pr-c |
| 7-439 | H | CH2 | CH2 | CH2 | CHNH(C═O)CH2Pr-c |
| 7-440 | H | CH2 | CH2 | CH2 | CHNH(C═O)CH2SMe |
| 7-441 | H | CH2 | CH2 | CH2 | CHNH(C═O)CH2S(═O)Me |
| 7-442 | H | CH2 | CH2 | CH2 | CHNH(C═O)CH2S(═O)2Me |
| 7-443 | H | CH2 | CH2 | CH2 | CHNH(C═O)NHCH3 |
| 7-444 | H | CH2 | CH2 | CH2 | CHNH(C═O)NHC2H5 |
| 7-445 | H | CH2 | CH2 | CH2 | CHNH(C═O)NHCH2CCH |
| 7-446 | H | CH2 | CH2 | CH2 | CHNHCO2C4H9-tert |
| 7-447 | H | CH2 | CH2 | CH2 | CHNHCO2CH3 |
| 7-448 | H | CH2 | CH2 | CH2 | CHNHCO2Ph |
| 7-449 | H | CH2 | CH2 | CH2 | CHO(C═O)CH3 |
| 7-450 | H | CH2 | CH2 | CH2 | CHN3 |
| 7-451 | H | CH2 | CH2 | CH2 | CHNH(C═S)NHC2H5 |
| 7-452 | H | CH2 | CH2 | CH2 | CHNH(C═S)C2H5 |
| 7-453 | H | CH2 | CH2 | CHNH2 | CH2 |
| 7-454 | H | CH2 | CH2 | CHNH(C═O)C2H5 | CH2 |
| 7-455 | H | CH2 | CH2 | CHNHCO2C4H9-tert | CH2 |
| 7-456 | H | CH2 | CH2 | C═O | CH2 |
| 7-457 | H | CH2 | CH2 | C═NOH | CH2 |
| 7-458 | H | CH | CH | CH | CNH2 |
| 7-459 | H | CH | CH | CH | CNH(C═O)CH3 |
| 7-460 | H | CH | CH | CH | CNH(C═O)C2H5 |
| 7-461 | H | CH | CH | CH | CNH(C═O)CH2CF3 |
| 7-462 | H | CH | CH | CH | CNH(C═O)Pr-c |
| 7-463 | H | CH | CH | CH | CNH(C═O)CH2Pr-c |
| 7-464 | H | CH | CH | CH | CNH(C═O)CH2SMe |
| 7-465 | H | CH | CH | CH | CNH(C═O)CH2S(═O)Me |
| 7-466 | H | CH | CH | CH | CNH(C═O)CH2S(═O)2Me |
| 7-467 | H | CH | CH | CH | CNH(C═O)NHCH3 |
| 7-468 | H | CH | CH | CH | CNH(C═O)NHC2H5 |
| 7-469 | H | CH | CH | CH | CNH(C═O)NHCH2CCH |
| 7-470 | H | CH | CH | CH | CNHCO2C4H9-tert |
| 7-471 | H | CH | CH | CH | CNHCO2CH3 |
| 7-472 | H | CH | CH | CH | CNHCO2Ph |
| 7-473 | H | CH | CH | CH | CHN(C═S)NHC2H5 |
| 7-474 | H | CH | CH | CH | CNH(C═S)C2H5 |
| 7-475 | H | CH | CH | CNH2 | CH |
| 7-476 | H | CH | CH | CNH(C═O)CH3 | CH |
| 7-477 | H | CH | CH | CNH(C═O)C2H5 | CH |
| 7-478 | H | CH | CH | CNH(C═O)Pr-c | CH |
| 7-479 | H | CH | CH | CNH(C═O)NHC2H5 | CH |
| 7-480 | H | CH | CH | CNHCO2C4H9-tert | CH |
| 7-481 | H | CH | N | N | CCl |
| 7-482 | H | CH | N | N | CNH2 |
| 7-483 | H | CH | N | N | CNH(C═O)CH3 |
| 7-484 | H | CH | N | N | CNH(C═O)C2H5 |
| 7-485 | H | CH | N | N | CNH(C═O)CH2CF3 |
| 7-486 | H | CH | N | N | CNH(C═O)Pr-c |
| 7-487 | H | CH | N | N | CNH(C═O)CH2Pr-c |
| 7-488 | H | CH | N | N | CNH(C═O)CH2SMe |
| 7-489 | H | CH | N | N | CNH(C═O)CH2S(═O)Me |
| 7-490 | H | CH | N | N | CNH(C═O)CH2S(═O)2Me |
| 7-491 | H | CH | N | N | CNH(C═O)NHCH3 |
| 7-492 | H | CH | N | N | CNH(C═O)NHC2H5 |
| 7-493 | H | CH | N | N | CNH(C═O)NHCH2CCH |
| 7-494 | H | CH | N | N | CHN(C═S)NHC2H5 |
| 7-495 | H | CH | N | N | CNH(C═S)C2H5 |
| 7-496 | H | CH | CH | N | CNH2 |
| 7-497 | H | CH | CH | N | CNH(C═O)CH3 |
| 7-498 | H | CH | CH | N | CNH(C═O)C2H5 |
| 7-499 | H | CH | CH | N | CNH(C═O)CH2CF3 |
| 7-500 | H | CH | CH | N | CNH(C═O)Pr-c |
| 7-501 | H | CH | CH | N | CNH(C═O)NHC2H5 |
| 7-502 | H | N | CH | CH | CNH2 |
| 7-503 | H | N | CH | CH | CNH(C═O)C2H5 |
| 7-504 | H | N | CH | CH | CNH(C═O)NHC2H5 |
| 7-505 | H | CH | N | CH | CNH2 |
| 7-506 | H | CH | N | CH | CNH(C═O)Pr-c |
| 7-507 | H | CH | N | CH | CNH(C═O)CH2S(═O)2Me |
| 7-508 | H | N | N | CH | CNH2 |
| 7-509 | H | N | N | CH | CNH(C═O)CH2CF3 |
| 7-510 | H | N | N | CH | CNH(C═O)CH2SMe |
| 7-511 | H | N | CH | N | CNH2 |
| 7-512 | H | N | CH | N | CNH(C═O)CH3 |
| 7-513 | H | N | CH | N | CNH(C═O)CH2S(═O)Me |
| 7-514 | H | CH | NCH3 | N | CNH2 |
| 7-515 | H | CH | NCH3 | N | CNH(C═O)C2H5 |
| 7-516 | H | CH | NCH3 | N | CNH(C═O)NHC2H5 |
| 7-517 | H | CH2 | O | N | CNH2 |
| 7-518 | H | CH2 | O | N | CNH(C═O)Pr-c |
| 7-519 | H | CH2 | O | N | CNH(C═O)CH2S(═O)2Me |
| 7-520 | H | CH2 | CH2 | CHCH3 | CHNH2 |
| 7-521 | H | CH2 | CH2 | CHCH3 | CHNH(C═O)CH3 |
| 7-522 | H | CH2 | CH2 | CHCH3 | CHNH(C═O)C2H5 |
| 7-523 | H | CH2 | CH2 | CHCH3 | CHNH(C═O)CH2CF3 |
| 7-524 | H | CH2 | CH2 | CHCH3 | CHNH(C═O)Pr-c |
| 7-525 | H | CH2 | CH2 | CHCH3 | CHNH(C═O)CH2Pr-c |
| 7-526 | H | CH2 | CH2 | CHCH3 | CHNH(C═O)CH2SMe |
| 7-527 | H | CH2 | CH2 | CHCH3 | CHNH(C═O)CH2S(═O)Me |
| 7-528 | H | CH2 | CH2 | CHCH3 | CHNH(C═O)CH2S(═O)2Me |
| 7-529 | H | CH2 | CH2 | CHCH3 | CHNH(C═O)NHCH3 |
| 7-530 | H | CH2 | CH2 | CHCH3 | CHNH(C═O)NHC2H5 |
| 7-531 | H | CH2 | CH2 | CHCH3 | CHNH(C═O)NHCH2CCH |
| 7-532 | H | CH2 | CH2 | CHCH3 | CHNHCO2C4H9-tert |
| 7-533 | H | CH2 | CH2 | CHCH3 | CHNHCO2CH3 |
| 7-534 | H | CH2 | CH2 | CHCH3 | CHNHCO2Ph |
| 7-535 | H | CH2 | CH2 | CHCH3 | CHN(C═S)NHC2H5 |
| 7-536 | H | CH2 | CH2 | CHCH3 | CHNH(C═S)C2H5 |
| 7-537 | H | CH2 | CH2 | C(CH3)2 | CNH2 |
| 7-538 | H | CH2 | CH2 | C(CH3)2 | CNH(C═O)CH3 |
| 7-539 | H | CH2 | CH2 | C(CH3)2 | CNH(C═O)NHC2H5 |
| 7-540 | H | CH2 | CH2 | C(CH3)2 | CNHCO2C4H9-tert |
| 7-541 | H | CH2 | CH2 | CHF | CNH2 |
| 7-542 | H | CH2 | CH2 | CHF | CNH(C═O)CH3 |
| 7-543 | H | CH2 | CH2 | CHF | CNH(C═O)NHC2H5 |
| 7-544 | H | CH2 | CH2 | CHF | CNHCO2C4H9-tert |
| 7-545 | H | CH2 | CH2 | CHCl | CNH2 |
| 7-546 | H | CH2 | CH2 | CHCl | CNH(C═O)CH3 |
| 7-547 | H | CH2 | CH2 | CHCl | CNH(C═O)NHC2H5 |
| 7-548 | H | CH2 | CH2 | CHCl | CNHCO2C4H9-tert |

TABLE 8

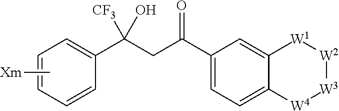

| Example | Xm | W¹ | W² | W³ | W⁴ |
|---|---|---|---|---|---|
| 8-1 | 3,5-Cl2 | — | CH2 | O | C═O |
| 8-2 | 3,5-Cl2 | — | CH2 | O | CH2 |
| 8-3 | 3,5-Cl2 | — | CH2 | CH2 | O |
| 8-4 | 3,5-Cl2 | — | O | CH2 | O |
| 8-5 | 3,5-Cl2 | — | O | CF2 | O |
| 8-6 | 3,5-Cl2 | — | CH | CH | O |
| 8-7 | 3,5-Cl2 | — | C═O | O | CH2 |
| 8-8 | 3,5-Cl2 | — | N | CH | O |
| 8-9 | 3,5-Cl2 | — | N | CH | S |
| 8-10 | 3,5-Cl2 | — | N | CH | NH |
| 8-11 | 3,5-Cl2 | — | O | CH | N |
| 8-12 | 3,5-Cl2 | — | S | CH | N |
| 8-13 | 3,5-Cl2 | — | CH | N | O |
| 8-14 | 3,5-Cl2 | — | CH | N | S |
| 8-15 | 3,5-Cl2 | — | CH2 | CH2 | C═O |
| 8-16 | 3,5-Cl2 | — | CH | CH | C═O |
| 8-17 | 3,5-Cl2 | — | CH2 | C═O | CH2 |
| 8-18 | 3,5-Cl2 | — | C═O | CH2 | CH2 |
| 8-19 | 3,5-Cl2 | — | CH2 | C═O | NCOCH3 |
| 8-20 | 3,5-Cl2 | — | CH2 | C═O | NCOCH2CF3 |
| 8-21 | 3,5-Cl2 | — | CH2 | CH2 | NCOCH3 |
| 8-22 | 3,5-Cl2 | — | CH2 | CH2 | NCOC2H5 |
| 8-23 | 3,5-Cl2 | — | CH2 | CH2 | NCOCH2CF3 |
| 8-24 | 3,5-Cl2 | — | CH2 | CH2 | N(C═O)NCH3 |
| 8-25 | 3,5-Cl2 | — | CH2 | CH2 | N(C═O)NC2H5 |
| 8-26 | 3,5-Cl2 | — | CH2 | CH2 | N(C═O)NCH2CCH |
| 8-27 | 3,5-Cl2 | — | CH2 | NCOCH3 | CH2 |
| 8-28 | 3,5-Cl2 | — | CH2 | NCOC2H5 | CH2 |
| 8-29 | 3,5-Cl2 | — | CH2 | NCOCH2CF3 | CH2 |
| 8-30 | 3,5-Cl2 | — | CH2 | CH2 | C═NOH |
| 8-31 | 3,5-Cl2 | — | CH2 | CH2 | C═NOCH3 |
| 8-32 | 3,5-Cl2 | — | CH2 | CH2 | C═NOCH2CF3 |
| 8-33 | 3,5-Cl2 | — | CH2 | CH2 | CHNHCOCH3 |
| 8-34 | 3,5-Cl2 | — | CH2 | CH2 | CHNHCOC2H5 |
| 8-35 | 3,5-Cl2 | — | CH2 | CH2 | CHNHCOCH2CF3 |
| 8-36 | 3,5-Cl2 | — | CH2 | CH2 | CHNHCOPr-c |
| 8-37 | 3,5-Cl2 | — | CH2 | CH2 | CHNHCOCH2Pr-c |
| 8-38 | 3,5-Cl2 | — | CH2 | CH2 | CHNHCOCH2SMe |
| 8-39 | 3,5-Cl2 | — | CH2 | CH2 | CHNHCOCH2SOMe |
| 8-40 | 3,5-Cl2 | — | CH2 | CH2 | CHNHCOCH2SO2Me |
| 8-41 | 3,5-Cl2 | — | CH2 | CH2 | CHNHC(═O)NHCH3 |
| 8-42 | 3,5-Cl2 | — | CH2 | CH2 | CHNHC(═O)NHC2H5 |
| 8-43 | 3,5-Cl2 | — | CH2 | CH2 | CHNHC(═O)NHCH2CCH |
| 8-44 | 3,5-Cl2 | — | CH2 | CH2 | CHNHCO2C4H9-t |
| 8-45 | 3,5-Cl2 | — | CH2 | CH2 | CHNHCO2CH3 |
| 8-46 | 3,5-Cl2 | — | CH2 | CH2 | CHNHCO2Ph |
| 8-47 | 3,5-Cl2 | — | CH2 | CH2 | CHOC(═O)CH3 |
| 8-48 | 3,5-Cl2 | — | CH2 | CH2 | CHN3 |
| 8-49 | 3,5-Cl2 | — | CH2 | CH2 | CHNHC(═S)NHC2H5 |
| 8-50 | 3,5-Cl2 | — | CH2 | CH2 | CHNHCSC2H5 |
| 8-51 | 3,5-Cl2 | — | CH2 | CHNHCOC2H5 | CH2 |
| 8-52 | 3,5-Cl2 | — | CH2 | CHNHCO2C4H9-t | CH2 |
| 8-53 | 3,5-Cl2 | — | CH2 | C═O | CH2 |
| 8-54 | 3,5-Cl2 | — | CH2 | C═NOH | CH2 |
| 8-55 | 3,5-Cl2 | — | O | N | CHNHCO2C4H9-t |
| 8-56 | 3,5-Cl2 | — | NCH3 | N | CHNHCO2C4H9-t |
| 8-57 | 3,5-Cl2 | — | SO2 | N | CHNHCO2C4H9-t |
| 8-58 | 3,5-Cl2 | — | CH | CH | CHNHCOCH3 |
| 8-59 | 3,5-Cl2 | — | CH | CH | CHNHCOC2H5 |
| 8-60 | 3,5-Cl2 | — | CH | CH | CHNHCOCH2CF3 |
| 8-61 | 3,5-Cl2 | — | CH | CH | CHNHCOPr-c |
| 8-62 | 3,5-Cl2 | — | CH | CH | CHNHCOCH2SMe |
| 8-63 | 3,5-Cl2 | — | CH | CH | CHNHCOCH2SOMe |
| 8-64 | 3,5-Cl2 | — | CH | CH | CHNHCOCH2SO2Me |
| 8-65 | 3,5-Cl2 | — | CH | CH | CHNHC(═O)NHC2H5 |
| 8-66 | 3,5-Cl2 | — | CH | CH | CHNHCO2C4H9-t |
| 8-67 | 3,5-Cl2 | — | CH | CH | CHOC(═O)CH3 |
| 8-68 | 3,5-Cl2 | — | CH | CH | CHN3 |
| 8-69 | 3,5-Cl2 | — | CH2 | CH2 | NNHCOCH3 |
| 8-70 | 3,5-Cl2 | — | CH2 | CH2 | NNHCOC2H5 |
| 8-71 | 3,5-Cl2 | — | CH2 | CH2 | NNHCOCH2CF3 |
| 8-72 | 3,5-Cl2 | — | CH2 | CH2 | NNHCOPr-c |
| 8-73 | 3,5-Cl2 | — | CH2 | CH2 | NNHCOCH2SMe |
| 8-74 | 3,5-Cl2 | — | CH2 | CH2 | NNHCOCH2SOMe |
| 8-75 | 3,5-Cl2 | — | CH2 | CH2 | NNHCOCH2SO2Me |
| 8-76 | 3,5-Cl2 | — | CH2 | CH2 | NNHC(═O)NHC2H5 |
| 8-77 | 3,5-Cl2 | — | CH2 | CH2 | NNHCO2C4H9-t |
| 8-78 | 3,5-Cl2 | — | CH2 | CHCH3 | NNHCO2C4H9-t |
| 8-79 | 3,5-Cl2 | — | CH2 | C(CH3)2 | NNHCO2C4H9-t |
| 8-80 | 3,5-Cl2 | — | CH2 | CHCl | NNHCO2C4H9-t |
| 8-81 | 3,5-CL2 | — | CH2 | CHF | NNHCO2C4H9-t |
| 8-82 | 3,4,5-CL3 | — | CH2 | O | C═O |
| 8-83 | 3,4,5-CL3 | — | CH2 | O | CH2 |
| 8-84 | 3,4,5-CL3 | — | CH2 | CH2 | O |
| 8-85 | 3,4,5-Cl3 | — | O | CH2 | O |
| 8-86 | 3,4,5-Cl3 | — | O | CF2 | O |
| 8-87 | 3,4,5-Cl3 | — | CH | CH | O |
| 8-88 | 3,4,5-Cl3 | — | C═O | O | CH2 |
| 8-89 | 3,4,5-Cl3 | — | N | CH | O |
| 8-90 | 3,4,5-Cl3 | — | N | CH | S |
| 8-91 | 3,4,5-Cl3 | — | N | CH | NH |
| 8-92 | 3,4,5-Cl3 | — | O | CH | N |
| 8-93 | 3,4,5-Cl3 | — | S | CH | N |
| 8-94 | 3,4,5-Cl3 | — | CH | N | O |
| 8-95 | 3,4,5-Cl3 | — | CH | N | S |
| 8-96 | 3,4,5-Cl3 | — | CH2 | CH2 | C═O |
| 8-97 | 3,4,5-Cl3 | — | CH | CH | C═O |
| 8-98 | 3,4,5-Cl3 | — | CH2 | C═O | CH2 |
| 8-99 | 3,4,5-Cl3 | — | C═O | CH2 | CH2 |
| 8-100 | 3,4,5-Cl3 | — | CH2 | C═O | NCOCH3 |
| 8-101 | 3,4,5-Cl3 | — | CH2 | C═O | NCOCH2CF3 |
| 8-102 | 3,4,5-Cl3 | — | CH2 | CH2 | NCOCH3 |
| 8-103 | 3,4,5-Cl3 | — | CH2 | CH2 | NCOC2H5 |
| 8-104 | 3,4,5-Cl3 | — | CH2 | CH2 | NCOCH2CF3 |
| 8-105 | 3,4,5-Cl3 | — | CH2 | CH2 | N(C═O)NCH3 |
| 8-106 | 3,4,5-Cl3 | — | CH2 | CH2 | N(C═O)NC2H5 |
| 8-107 | 3,4,5-Cl3 | — | CH2 | CH2 | N(C═O)NCH2CCH |
| 8-108 | 3,4,5-Cl3 | — | CH2 | NCOCH3 | CH2 |
| 8-109 | 3,4,5-Cl3 | — | CH2 | NCOC2H5 | CH2 |
| 8-110 | 3,4,5-Cl3 | — | CH2 | NCOCH2CF3 | CH2 |
| 8-111 | 3,4,5-Cl3 | — | CH2 | CH2 | C═NOH |
| 8-112 | 3,4,5-Cl3 | — | CH2 | CH2 | C═NOCH3 |
| 8-113 | 3,4,5-Cl3 | — | CH2 | CH2 | C═NOCH2CF3 |
| 8-114 | 3,4,5-Cl3 | — | CH2 | CH2 | CHNHCOCH3 |
| 8-115 | 3,4,5-Cl3 | — | CH2 | CH2 | CHNHCOC2H5 |
| 8-116 | 3,4,5-Cl3 | — | CH2 | CH2 | CHNHCOCH2CF3 |
| 8-117 | 3,4,5-Cl3 | — | CH2 | CH2 | CHNHCOPr-c |
| 8-118 | 3,4,5-Cl3 | — | CH2 | CH2 | CHNHCOCH2Pr-c |
| 8-119 | 3,4,5-Cl3 | — | CH2 | CH2 | CHNHCOCH2SMe |
| 8-120 | 3,4,5-Cl3 | — | CH2 | CH2 | CHNHCOCH2SOMe |
| 8-121 | 3,4,5-Cl3 | — | CH2 | CH2 | CHNHCOCH2SO2Me |
| 8-122 | 3,4,5-Cl3 | — | CH2 | CH2 | CHNHC(═O)NHCH3 |
| 8-123 | 3,4,5-Cl3 | — | CH2 | CH2 | CHNHC(═O)NHC2H5 |
| 8-124 | 3,4,5-Cl3 | — | CH2 | CH2 | CHNHC(═O)NHCH2CCH |
| 8-125 | 3,4,5-Cl3 | — | CH2 | CH2 | CHNHCO2C4H9-t |
| 8-126 | 3,4,5-Cl3 | — | CH2 | CH2 | CHNHCO2CH3 |
| 8-127 | 3,4,5-Cl3 | — | CH2 | CH2 | CHNHCO2Ph |
| 8-128 | 3,4,5-Cl3 | — | CH2 | CH2 | CHOC(═O)CH3 |
| 8-129 | 3,4,5-Cl3 | — | CH2 | CH2 | CHN3 |
| 8-130 | 3,4,5-Cl3 | — | CH2 | CH2 | CHNHC(═S)NHC2H5 |
| 8-131 | 3,4,5-Cl3 | — | CH2 | CH2 | CHNHCSC2H5 |
| 8-132 | 3,4,5-Cl3 | — | CH2 | CHNHCOC2H5 | CH2 |
| 8-133 | 3,4,5-Cl3 | — | CH2 | CHNHCO2C4H9-t | CH2 |
| 8-134 | 3,4,5-Cl3 | — | CH2 | C═O | CH2 |
| 8-135 | 3,4,5-Cl3 | — | CH2 | C═NOH | CH2 |
| 8-136 | 3,4,5-Cl3 | — | O | N | CHNHCO2C4H9-t |
| 8-137 | 3,4,5-Cl3 | — | NCH3 | N | CHNHCO2C4H9-t |
| 8-138 | 3,4,5-Cl3 | — | SO2 | N | CHNHCO2C4H9-t |
| 8-139 | 3,4,5-Cl3 | — | CH | CH | CHNHCOCH3 |
| 8-140 | 3,4,5-Cl3 | — | CH | CH | CHNHCOC2H5 |
| 8-141 | 3,4,5-Cl3 | — | CH | CH | CHNHCOCH2CF3 |
| 8-142 | 3,4,5-Cl3 | — | CH | CH | CHNHCOPr-c |
| 8-143 | 3,4,5-Cl3 | — | CH | CH | CHNHCOCH2SMe |
| 8-144 | 3,4,5-Cl3 | — | CH | CH | CHNHCOCH2SOMe |
| 8-145 | 3,4,5-Cl3 | — | CH | CH | CHNHCOCH2SO2Me |
| 8-146 | 3,4,5-Cl3 | — | CH | CH | CHNHC(═O)NHC2H5 |
| 8-147 | 3,4,5-Cl3 | — | CH | CH | CHNHCO2C4H9-t |
| 8-148 | 3,4,5-Cl3 | — | CH | CH | CHOC(═O)CH3 |
| 8-149 | 3,4,5-Cl3 | — | CH | CH | CHN3 |
| 8-150 | 3,4,5-Cl3 | — | CH2 | CH2 | NNHCOCH3 |
| 8-151 | 3,4,5-Cl3 | — | CH2 | CH2 | NNHCOC2H5 |
| 8-152 | 3,4,5-Cl3 | — | CH2 | CH2 | NNHCOCH2CF3 |
| 8-153 | 3,4,5-Cl3 | — | CH2 | CH2 | NNHCOPr-c |
| 8-154 | 3,4,5-Cl3 | — | CH2 | CH2 | NNHCOCH2SMe |
| 8-155 | 3,4,5-Cl3 | — | CH2 | CH2 | NNHCOCH2SOMe |
| 8-156 | 3,4,5-Cl3 | — | CH2 | CH2 | NNHCOCH2SO2Me |
| 8-157 | 3,4,5-Cl3 | — | CH2 | CH2 | NNHC(═O)NHC2H5 |
| 8-158 | 3,4,5-Cl3 | — | CH2 | CH2 | NNHCO2C4H9-t |
| 8-159 | 3,4,5-Cl3 | — | CH2 | CHCH3 | NNHCO2C4H9-t |
| 8-160 | 3,4,5-Cl3 | — | CH2 | C(CH3)2 | NNHCO2C4H9-t |
| 8-161 | 3,4,5-Cl3 | — | CH2 | CHCl | NNHCO2C4H9-t |
| 8-162 | 3,4,5-Cl3 | — | CH2 | CHF | NNHCO2C4H9-t |
| 8-163 | 3,5-(CF3)2 | — | CH2 | CH2 | CHNHCOCH3 |
| 8-164 | 3,5-(CF3)2 | — | CH2 | CH2 | CHNHCOC2H5 |
| 8-165 | 3,5-(CF3)2 | — | CH2 | CH2 | CHNHCOCH2CF3 |
| 8-166 | 3,5-(CF3)2 | — | CH2 | CH2 | CHNHCOPr-c |
| 8-167 | 3,5-(CF3)2 | — | CH2 | CH2 | CHNHCOCH2SO2Me |
| 8-168 | 3,5-(CF3)2 | — | CH2 | CH2 | CHNHC(═O)NHC2H5 |
| 8-169 | 3,5-(CF3)2 | — | CH2 | CH2 | CHNHCO2C4H9-t |
| 8-170 | 3-CF3 | — | CH2 | CH2 | CHNHCOCH3 |
| 8-171 | 3-CF3 | — | CH2 | CH2 | CHNHCOC2H5 |
| 8-172 | 3-CF3 | — | CH2 | CH2 | CHNHCOCH2CF3 |
| 8-173 | 3-CF3 | — | CH2 | CH2 | CHNHCOPr-c |
| 8-174 | 3-CF3 | — | CH2 | CH2 | CHNHCOCH2SO2Me |
| 8-175 | 3-CF3 | — | CH2 | CH2 | CHNHC(═O)NHC2H5 |
| 8-176 | 3-CF3 | — | CH2 | CH2 | CHNHCO2C4H9-t |
| 8-177 | 3,5-Cl2 | CH2 | CH2 | O | C═O |
| 8-178 | 3,5-Cl2 | CH2 | C═O | O | CH2 |
| 8-179 | 3,5-Cl2 | CH | N | CH | O |
| 8-180 | 3,5-Cl2 | CH | N | CH | S |
| 8-181 | 3,5-Cl2 | CH | N | CH | NH |
| 8-182 | 3,5-Cl2 | CH2 | CH2 | CH2 | C═O |
| 8-183 | 3,5-Cl2 | CH2 | CH2 | C═O | CH2 |
| 8-184 | 3,5-Cl2 | CH2 | C═O | CH2 | CH2 |
| 8-185 | 3,5-Cl2 | CH2 | CH2 | C═O | NCOCH3 |
| 8-186 | 3,5-Cl2 | CH2 | CH2 | C═O | NCOCH2CF3 |
| 8-187 | 3,5-Cl2 | CH2 | CH2 | CH2 | NCOCH3 |
| 8-188 | 3,5-Cl2 | CH2 | CH2 | CH2 | NCOC2H5 |
| 8-189 | 3,5-Cl2 | CH2 | CH2 | CH2 | NCOCH2CF3 |
| 8-190 | 3,5-Cl2 | CH2 | CH2 | CH2 | N(C═O)NCH3 |
| 8-191 | 3,5-Cl2 | CH2 | CH2 | CH2 | N(C═O)NC2H5 |
| 8-192 | 3,5-Cl2 | CH2 | CH2 | CH2 | N(C═O)NCH2CCH |
| 8-193 | 3,5-Cl2 | CH2 | CH2 | NCOCH3 | CH2 |
| 8-194 | 3,5-Cl2 | CH2 | CH2 | NCOC2H5 | CH2 |
| 8-195 | 3,5-Cl2 | CH2 | CH2 | NCOCH2CF3 | CH2 |
| 8-196 | 3,5-Cl2 | CH2 | CH2 | CH2 | C═NOH |
| 8-197 | 3,5-Cl2 | CH2 | CH2 | CH2 | C═NOCH3 |
| 8-198 | 3,5-Cl2 | CH2 | CH2 | CH2 | C═NOCH2CF3 |
| 8-199 | 3,5-Cl2 | CH2 | CH2 | CH2 | CHNHCOCH3 |
| 8-200 | 3,5-Cl2 | CH2 | CH2 | CH2 | CHNHCOC2H5 |
| 8-201 | 3,5-Cl2 | CH2 | CH2 | CH2 | CHNHCOCH2CF3 |
| 8-202 | 3,5-Cl2 | CH2 | CH2 | CH2 | CHNHCOPr-c |
| 8-203 | 3,5-Cl2 | CH2 | CH2 | CH2 | CHNHCOCH2Pr-c |
| 8-204 | 3,5-Cl2 | CH2 | CH2 | CH2 | CHNHCOCH2SMe |
| 8-205 | 3,5-Cl2 | CH2 | CH2 | CH2 | CHNHCOCH2SOMe |
| 8-206 | 3,5-Cl2 | CH2 | CH2 | CH2 | CHNHCOCH2SO2Me |
| 8-207 | 3,5-Cl2 | CH2 | CH2 | CH2 | CHNHC(═O)NHCH3 |
| 8-208 | 3,5-Cl2 | CH2 | CH2 | CH2 | CHNHC(═O)NHC2H5 |
| 8-209 | 3,5-Cl2 | CH2 | CH2 | CH2 | CHNHC(═O)NHCH2CCH |
| 8-210 | 3,5-Cl2 | CH2 | CH2 | CH2 | CHNHCO2C4H9-t |
| 8-211 | 3,5-Cl2 | CH2 | CH2 | CH2 | CHNHCO2CH3 |
| 8-212 | 3,5-Cl2 | CH2 | CH2 | CH2 | CHNHCO2Ph |
| 8-213 | 3,5-Cl2 | CH2 | CH2 | CH2 | CHOC(═O)CH3 |
| 8-214 | 3,5-Cl2 | CH2 | CH2 | CH2 | CHN3 |
| 8-215 | 3,5-Cl2 | CH2 | CH2 | CH2 | CHNHC(═S)NHC2H5 |
| 8-216 | 3,5-Cl2 | CH2 | CH2 | CH2 | CHNHCSC2H5 |
| 8-217 | 3,5-Cl2 | CH2 | CH2 | CHNHCOC2H5 | CH2 |
| 8-218 | 3,5-Cl2 | CH2 | CH2 | CHNHCO2C4H9-t | CH2 |
| 8-219 | 3,5-Cl2 | CH2 | CH2 | C═O | CH2 |
| 8-220 | 3,5-Cl2 | CH2 | CH2 | C═NOH | CH2 |
| 8-221 | 3,5-Cl2 | CH | CH | CH | CNHCOCH3 |
| 8-222 | 3,5-Cl2 | CH | CH | CH | CNHCOC2H5 |
| 8-223 | 3,5-Cl2 | CH | CH | CH | CNHCOCH2CF3 |
| 8-224 | 3,5-Cl2 | CH | CH | CH | CNHCOPr-c |
| 8-225 | 3,5-Cl2 | CH | CH | CH | CNHCOCH2SMe |
| 8-226 | 3,5-Cl2 | CH | CH | CH | CNHCOCH2SOMe |
| 8-227 | 3,5-Cl2 | CH | CH | CH | CNHCOCH2SO2Me |
| 8-228 | 3,5-Cl2 | CH | CH | CH | CNHC(═O)NHC2H5 |
| 8-229 | 3,5-Cl2 | CH | CH | CH | CNHCO2C4H9-t |
| 8-230 | 3,5-Cl2 | CH | CH | CH | COC(═O)CH3 |
| 8-231 | 3,5-Cl2 | CH | CH | CH | CN3 |
| 8-232 | 3,5-Cl2 | CH2 | CH2 | CH2 | NNHCOCH3 |
| 8-233 | 3,5-Cl2 | CH2 | CH2 | CH2 | NNHCOC2H5 |
| 8-234 | 3,5-Cl2 | CH2 | CH2 | CH2 | NNHCOCH2CF3 |
| 8-235 | 3,5-Cl2 | CH2 | CH2 | CH2 | NNHCOPr-c |
| 8-236 | 3,5-Cl2 | CH2 | CH2 | CH2 | NNHCOCH2SMe |
| 8-237 | 3,5-Cl2 | CH2 | CH2 | CH2 | NNHCOCH2SOMe |
| 8-238 | 3,5-Cl2 | CH2 | CH2 | CH2 | NNHCOCH2SO2Me |
| 8-239 | 3,5-Cl2 | CH2 | CH2 | CH2 | NNHC(═O)NHC2H5 |
| 8-240 | 3,5-Cl2 | CH2 | CH2 | CH2 | NNHCO2C4H9-t |
| 8-241 | 3,5-Cl2 | CH2 | CH2 | CHCH3 | NNHCO2C4H9-t |
| 8-242 | 3,5-Cl2 | CH2 | CH2 | C(CH3)2 | NNHCO2C4H9-t |
| 8-243 | 3,5-Cl2 | CH2 | CH2 | CHCl | NNHCO2C4H9-t |
| 8-244 | 3,5-Cl2 | CH2 | CH2 | CHF | NNHCO2C4H9-t |
| 8-245 | 3,4,5-Cl3 | CH2 | CH2 | O | C═O |
| 8-246 | 3,4,5-Cl3 | CH2 | CH2 | O | CH2 |
| 8-247 | 3,4,5-Cl3 | CH2 | CH2 | CH2 | O |
| 8-248 | 3,4,5-Cl3 | CH2 | C═O | O | CH2 |
| 8-249 | 3,4,5-Cl3 | CH2 | CH2 | CH2 | C═O |
| 8-250 | 3,4,5-Cl3 | CH2 | CH | CH | C═O |
| 8-251 | 3,4,5-Cl3 | CH2 | CH2 | C═O | CH2 |
| 8-252 | 3,4,5-Cl3 | CH2 | C═O | CH2 | CH2 |
| 8-253 | 3,4,5-Cl3 | CH2 | CH2 | C═O | NCOCH3 |
| 8-254 | 3,4,5-Cl3 | CH2 | CH2 | C═O | NCOCH2CF3 |
| 8-255 | 3,4,5-Cl3 | CH2 | CH2 | CH2 | NCOCH3 |
| 8-256 | 3,4,5-Cl3 | CH2 | CH2 | CH2 | NCOC2H5 |
| 8-257 | 3,4,5-Cl3 | CH2 | CH2 | CH2 | NCOCH2CF3 |
| 8-258 | 3,4,5-Cl3 | CH2 | CH2 | CH2 | N(C═O)NCH3 |
| 8-259 | 3,4,5-Cl3 | CH2 | CH2 | CH2 | N(C═O)NC2H5 |
| 8-260 | 3,4,5-Cl3 | CH2 | CH2 | CH2 | N(C═O)NCH2CCH |
| 8-261 | 3,4,5-Cl3 | CH2 | CH2 | NCOCH3 | CH2 |
| 8-262 | 3,4,5-Cl3 | CH2 | CH2 | NCOC2H5 | CH2 |
| 8-263 | 3,4,5-Cl3 | CH2 | CH2 | NCOCH2CF3 | CH2 |
| 8-264 | 3,4,5-Cl3 | CH2 | CH2 | CH2 | C═NOH |
| 8-265 | 3,4,5-Cl3 | CH2 | CH2 | CH2 | C═NOCH3 |
| 8-266 | 3,4,5-Cl3 | CH2 | CH2 | CH2 | C═NOCH2CF3 |
| 8-267 | 3,4,5-Cl3 | CH2 | CH2 | CH2 | CHNHCOCH3 |
| 8-268 | 3,4,5-Cl3 | CH2 | CH2 | CH2 | CHNHCOC2H5 |
| 8-269 | 3,4,5-Cl3 | CH2 | CH2 | CH2 | CHNHCOCH2CF3 |
| 8-270 | 3,4,5-Cl3 | CH2 | CH2 | CH2 | CHNHCOPr-c |
| 8-271 | 3,4,5-Cl3 | CH2 | CH2 | CH2 | CHNHCOCH2Pr-c |
| 8-272 | 3,4,5-Cl3 | CH2 | CH2 | CH2 | CHNHCOCH2SMe |
| 8-273 | 3,4,5-Cl3 | CH2 | CH2 | CH2 | CHNHCOCH2SOMe |
| 8-274 | 3,4,5-Cl3 | CH2 | CH2 | CH2 | CHNHCOCH2SO2Me |
| 8-275 | 3,4,5-Cl3 | CH2 | CH2 | CH2 | CHNHC(═O)NHCH3 |
| 8-276 | 3,4,5-Cl3 | CH2 | CH2 | CH2 | CHNHC(═O)NHC2H5 |
| 8-277 | 3,4,5-Cl3 | CH2 | CH2 | CH2 | CHNHC(═O)NHCH2CCH |
| 8-278 | 3,4,5-Cl3 | CH2 | CH2 | CH2 | CHNHCO2C4H9-t |
| 8-279 | 3,4,5-Cl3 | CH2 | CH2 | CH2 | CHNHCO2CH3 |
| 8-280 | 3,4,5-Cl3 | CH2 | CH2 | CH2 | CHNHCO2Ph |
| 8-281 | 3,4,5-Cl3 | CH2 | CH2 | CH2 | CHOC(═O)CH3 |
| 8-282 | 3,4,5-Cl3 | CH2 | CH2 | CH2 | CHN3 |
| 8-283 | 3,4,5-Cl3 | CH2 | CH2 | CH2 | CHNHC(═S)NHC2H5 |
| 8-284 | 3,4,5-Cl3 | CH2 | CH2 | CH2 | CHNHCSC2H5 |
| 8-285 | 3,4,5-Cl3 | CH2 | CH2 | CHNHCOC2H5 | CH2 |
| 8-286 | 3,4,5-Cl3 | CH2 | CH2 | CHNHCO2C4H9-t | CH2 |
| 8-287 | 3,4,5-Cl3 | CH2 | CH2 | C═O | CH2 |
| 8-288 | 3,4,5-Cl3 | CH2 | CH2 | C═NOH | CH2 |
| 8-289 | 3,4,5-Cl3 | CH | CH | CH | CNHCOCH3 |
| 8-290 | 3,4,5-Cl3 | CH | CH | CH | CNHCOC2H5 |
| 8-291 | 3,4,5-Cl3 | CH | CH | CH | CNHCOCH2CF3 |
| 8-292 | 3,4,5-Cl3 | CH | CH | CH | CNHCOPr-c |
| 8-293 | 3,4,5-Cl3 | CH | CH | CH | CNHCOCH2SMe |
| 8-294 | 3,4,5-Cl3 | CH | CH | CH | CNHCOCH2SOMe |
| 8-295 | 3,4,5-Cl3 | CH | CH | CH | CNHCOCH2SO2Me |
| 8-296 | 3,4,5-Cl3 | CH | CH | CH | CNHC(═O)NHC2H5 |
| 8-297 | 3,4,5-Cl3 | CH | CH | CH | CNHCO2C4H9-t |
| 8-298 | 3,4,5-Cl3 | CH | CH | CH | COC(═O)CH3 |
| 8-299 | 3,4,5-Cl3 | CH | CH | CH | CN3 |
| 8-300 | 3,4,5-Cl3 | CH2 | CH2 | CH2 | NNHCOCH3 |
| 8-301 | 3,4,5-Cl3 | CH2 | CH2 | CH2 | NNHCOC2H5 |
| 8-302 | 3,4,5-Cl3 | CH2 | CH2 | CH2 | NNHCOCH2CF3 |
| 8-303 | 3,4,5-Cl3 | CH2 | CH2 | CH2 | NNHCOPr-c |
| 8-304 | 3,4,5-Cl3 | CH2 | CH2 | CH2 | NNHCOCH2SMe |
| 8-305 | 3,4,5-Cl3 | CH2 | CH2 | CH2 | NNHCOCH2SOMe |
| 8-306 | 3,4,5-Cl3 | CH2 | CH2 | CH2 | NNHCOCH2SO2Me |
| 8-307 | 3,4,5-Cl3 | CH2 | CH2 | CH2 | NNHC(═O)NHC2H5 |
| 8-308 | 3,4,5-Cl3 | CH2 | CH2 | CH2 | NNHCO2C4H9-t |
| 8-309 | 3,4,5-Cl3 | CH2 | CH2 | CHCH3 | NNHCO2C4H9-t |
| 8-310 | 3,4,5-Cl3 | CH2 | CH2 | C(CH3)2 | NNHCO2C4H9-t |
| 8-311 | 3,4,5-Cl3 | CH2 | CH2 | CHCL | NNHCO2C4H9-t |
| 8-312 | 3,4,5-Cl3 | CH2 | CH2 | CHF | NNHCO2C4H9-t |
| 8-313 | 3,5-(CF3)2 | CH2 | CH2 | CH2 | CHNHCOCH3 |
| 8-314 | 3,5-(CF3)2 | CH2 | CH2 | CH2 | CHNHCOC2H5 |
| 8-315 | 3,5-(CF3)2 | CH2 | CH2 | CH2 | CHNHCOCH2CF3 |
| 8-316 | 3,5-(CF3)2 | CH2 | CH2 | CH2 | CHNHCOPr-c |
| 8-317 | 3,5-(CF3)2 | CH2 | CH2 | CH2 | CHNHCOCH2SO2Me |
| 8-318 | 3,5-(CF3)2 | CH2 | CH2 | CH2 | CHNHC(═O)NHC2H5 |
| 8-319 | 3,5-(CF3)2 | CH2 | CH2 | CH2 | CHNHCO2C4H9-t |
| 8-320 | 3-CF3 | CH2 | CH2 | CH2 | CHNHCOCH3 |
| 8-321 | 3-CF3 | CH2 | CH2 | CH2 | CHNHCOC2H5 |
| 8-322 | 3-CF3 | CH2 | CH2 | CH2 | CHNHCOCH2CF3 |
| 8-323 | 3-CF3 | CH2 | CH2 | CH2 | CHNHCOPr-c |
| 8-324 | 3-CF3 | CH2 | CH2 | CH2 | CHNHCOCH2SO2Me |
| 8-325 | 3-CF3 | CH2 | CH2 | CH2 | CHNHC(═O)NHC2H5 |
| 8-326 | 3-CF3 | CH2 | CH2 | CH2 | CHNHCO2C4H9-t |

TABLE 9

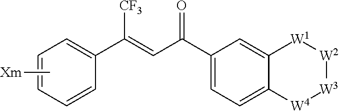

| Example | Xm | W¹ | W² | W³ | W⁴ |
|---|---|---|---|---|---|
| 9-1 | 3,5-Cl2 | — | CH2 | O | C═O |
| 9-2 | 3,5-Cl2 | — | CH2 | O | CH2 |
| 9-3 | 3,5-Cl2 | — | CH2 | CH2 | O |
| 9-4 | 3,5-Cl2 | — | O | CH2 | O |
| 9-5 | 3,5-Cl2 | — | O | CF2 | O |
| 9-6 | 3,5-Cl2 | — | CH | CH | O |
| 9-7 | 3,5-Cl2 | — | C═O | O | CH2 |
| 9-8 | 3,5-Cl2 | — | N | CH | O |
| 9-9 | 3,5-Cl2 | — | N | CH | S |
| 9-10 | 3,5-Cl2 | — | N | CH | NH |
| 9-11 | 3,5-Cl2 | — | O | CH | N |
| 9-12 | 3,5-Cl2 | — | S | CH | N |
| 9-13 | 3,5-Cl2 | — | CH | N | O |
| 9-14 | 3,5-Cl2 | — | CH | N | S |
| 9-15 | 3,5-Cl2 | — | CH2 | CH2 | C═O |
| 9-16 | 3,5-Cl2 | — | CH | CH | C═O |
| 9-17 | 3,5-Cl2 | — | CH2 | C═O | CH2 |
| 9-18 | 3,5-Cl2 | — | C═O | CH2 | CH2 |
| 9-19 | 3,5-Cl2 | — | CH2 | C═O | NCOCH3 |
| 9-20 | 3,5-Cl2 | — | CH2 | C═O | NCOCH2CF3 |
| 9-21 | 3,5-Cl2 | — | CH2 | CH2 | NCOCH3 |
| 9-22 | 3,5-Cl2 | — | CH2 | CH2 | NCOC2H5 |
| 9-23 | 3,5-Cl2 | — | CH2 | CH2 | NCOCH2CF3 |
| 9-24 | 3,5-Cl2 | — | CH2 | CH2 | N(C═O)NCH3 |
| 9-25 | 3,5-Cl2 | — | CH2 | CH2 | N(C═O)NC2H5 |
| 9-26 | 3,5-Cl2 | — | CH2 | CH2 | N(C═O)NCH2CCH |
| 9-27 | 3,5-Cl2 | — | CH2 | NCOCH3 | CH2 |
| 9-28 | 3,5-Cl2 | — | CH2 | NCOC2H5 | CH2 |
| 9-29 | 3,5-Cl2 | — | CH2 | NCOCH2CF3 | CH2 |
| 9-30 | 3,5-Cl2 | — | CH2 | CH2 | C═NOH |
| 9-31 | 3,5-Cl2 | — | CH2 | CH2 | C═NOCH3 |
| 9-32 | 3,5-Cl2 | — | CH2 | CH2 | C═NOCH2CF3 |
| 9-33 | 3,5-Cl2 | — | CH2 | CH2 | CHNHCOCH3 |
| 9-34 | 3,5-Cl2 | — | CH2 | CH2 | CHNHCOC2H5 |
| 9-35 | 3,5-Cl2 | — | CH2 | CH2 | CHNHCOCH2CF3 |
| 9-36 | 3,5-Cl2 | — | CH2 | CH2 | CHNHCOPr-c |
| 9-37 | 3,5-Cl2 | — | CH2 | CH2 | CHNHCOCH2Pr-c |
| 9-38 | 3,5-Cl2 | — | CH2 | CH2 | CHNHCOCH2SMe |
| 9-39 | 3,5-Cl2 | — | CH2 | CH2 | CHNHCOCH2SOMe |
| 9-40 | 3,5-Cl2 | — | CH2 | CH2 | CHNHCOCH2SO2Me |
| 9-41 | 3,5-Cl2 | — | CH2 | CH2 | CHNHC(═O)NHCH3 |
| 9-42 | 3,5-Cl2 | — | CH2 | CH2 | CHNHC(═O)NHC2H5 |
| 9-43 | 3,5-Cl2 | — | CH2 | CH2 | CHNHC(═O)NHCH2CCH |
| 9-44 | 3,5-Cl2 | — | CH2 | CH2 | CHNHCO2C4H9-t |
| 9-45 | 3,5-Cl2 | — | CH2 | CH2 | CHNHCO2CH3 |
| 9-46 | 3,5-Cl2 | — | CH2 | CH2 | CHNHCO2Ph |
| 9-47 | 3,5-Cl2 | — | CH2 | CH2 | CHOC(═O)CH3 |
| 9-48 | 3,5-Cl2 | — | CH2 | CH2 | CHN3 |
| 9-49 | 3,5-Cl2 | — | CH2 | CH2 | CHNHC(═S)NHC2H5 |
| 9-50 | 3,5-Cl2 | — | CH2 | CH2 | CHNHCSC2H5 |
| 9-51 | 3,5-Cl2 | — | CH2 | CHNHCOC2H5 | CH2 |
| 9-52 | 3,5-Cl2 | — | CH2 | CHNHCO2C4H9-t | CH2 |
| 9-53 | 3,5-Cl2 | — | CH2 | C═O | CH2 |
| 9-54 | 3,5-Cl2 | — | CH2 | C═NOH | CH2 |
| 9-55 | 3,5-Cl2 | — | O | N | CHNHCO2C4H9-t |
| 9-56 | 3,5-Cl2 | — | NCH3 | N | CHNHCO2C4H9-t |
| 9-57 | 3,5-Cl2 | — | SO2 | N | CHNHCO2C4H9-t |
| 9-58 | 3,5-Cl2 | — | CH | CH | CHNHCOCH3 |
| 9-59 | 3,5-Cl2 | — | CH | CH | CHNHCOC2H5 |
| 9-60 | 3,5-Cl2 | — | CH | CH | CHNHCOCH2CF3 |
| 9-61 | 3,5-Cl2 | — | CH | CH | CHNHCOPr-c |
| 9-62 | 3,5-Cl2 | — | CH | CH | CHNHCOCH2SMe |
| 9-63 | 3,5-Cl2 | — | CH | CH | CHNHCOCH2SOMe |
| 9-64 | 3,5-Cl2 | — | CH | CH | CHNHCOCH2SO2Me |
| 9-65 | 3,5-Cl2 | — | CH | CH | CHNHC(═O)NHC2H5 |
| 9-66 | 3,5-Cl2 | — | CH | CH | CHNHCO2C4H9-t |
| 9-67 | 3,5-Cl2 | — | CH | CH | CHOC(═O)CH3 |
| 9-68 | 3,5-Cl2 | — | CH | CH | CHN3 |
| 9-69 | 3,5-Cl2 | — | CH2 | CH2 | NNHCOCH3 |
| 9-70 | 3,5-Cl2 | — | CH2 | CH2 | NNHCOC2H5 |
| 9-71 | 3,5-Cl2 | — | CH2 | CH2 | NNHCOCH2CF3 |
| 9-72 | 3,5-Cl2 | — | CH2 | CH2 | NNHCOPr-c |
| 9-73 | 3,5-Cl2 | — | CH2 | CH2 | NNHCOCH2SMe |
| 9-74 | 3,5-Cl2 | — | CH2 | CH2 | NNHCOCH2SOMe |
| 9-75 | 3,5-Cl2 | — | CH2 | CH2 | NNHCOCH2SO2Me |
| 9-76 | 3,5-Cl2 | — | CH2 | CH2 | NNHC(═O)NHC2H5 |
| 9-77 | 3,5-Cl2 | — | CH2 | CH2 | NNHCO2C4H9-t |
| 9-78 | 3,5-Cl2 | — | CH2 | CHCH3 | NNHCO2C4H9-t |
| 9-79 | 3,5-Cl2 | — | CH2 | C(CH3)2 | NNHCO2C4H9-t |
| 9-80 | 3,5-Cl2 | — | CH2 | CHCl | NNHCO2C4H9-t |
| 9-81 | 3,5-Cl2 | — | CH2 | CHF | NNHCO2C4H9-t |
| 9-82 | 3,4,5-Cl3 | — | CH2 | O | C═O |
| 9-83 | 3,4,5-Cl3 | — | CH2 | O | CH2 |
| 9-84 | 3,4,5-Cl3 | — | CH2 | CH2 | O |
| 9-85 | 3,4,5-Cl3 | — | O | CH2 | O |
| 9-86 | 3,4,5-Cl3 | — | O | CF2 | O |
| 9-87 | 3,4,5-Cl3 | — | CH | CH | O |
| 9-88 | 3,4,5-Cl3 | — | C═O | O | CH2 |
| 9-89 | 3,4,5-Cl3 | — | N | CH | O |
| 9-90 | 3,4,5-Cl3 | — | N | CH | S |
| 9-91 | 3,4,5-Cl3 | — | N | CH | NH |
| 9-92 | 3,4,5-Cl3 | — | O | CH | N |
| 9-93 | 3,4,5-Cl3 | — | S | CH | N |
| 9-94 | 3,4,5-Cl3 | — | CH | N | O |
| 9-95 | 3,4,5-Cl3 | — | CH | N | S |
| 9-96 | 3,4,5-Cl3 | — | CH2 | CH2 | C═O |
| 9-97 | 3,4,5-Cl3 | — | CH | CH | C═O |
| 9-98 | 3,4,5-Cl3 | — | CH2 | C═O | CH2 |
| 9-99 | 3,4,5-Cl3 | — | C═O | CH2 | CH2 |
| 9-100 | 3,4,5-Cl3 | — | CH2 | C═O | NCOCH3 |
| 9-101 | 3,4,5-Cl3 | — | CH2 | C═O | NCOCH2CF3 |
| 9-102 | 3,4,5-Cl3 | — | CH2 | CH2 | NCOCH3 |
| 9-103 | 3,4,5-Cl3 | — | CH2 | CH2 | NCOC2H5 |
| 9-104 | 3,4,5-Cl3 | — | CH2 | CH2 | NCOCH2CF3 |
| 9-105 | 3,4,5-Cl3 | — | CH2 | CH2 | N(C═O)NCH3 |
| 9-106 | 3,4,5-Cl3 | — | CH2 | CH2 | N(C═O)NC2H5 |
| 9-107 | 3,4,5-Cl3 | — | CH2 | CH2 | N(C═O)NCH2CCH |
| 9-108 | 3,4,5-Cl3 | — | CH2 | NCOCH3 | CH2 |
| 9-109 | 3,4,5-Cl3 | — | CH2 | NCOC2H5 | CH2 |
| 9-110 | 3,4,5-Cl3 | — | CH2 | NCOCH2CF3 | CH2 |
| 9-111 | 3,4,5-Cl3 | — | CH2 | CH2 | C═NOH |
| 9-112 | 3,4,5-Cl3 | — | CH2 | CH2 | C═NOCH3 |
| 9-113 | 3,4,5-Cl3 | — | CH2 | CH2 | C═NOCH2CF3 |
| 9-114 | 3,4,5-Cl3 | — | CH2 | CH2 | CHNHCOCH3 |
| 9-115 | 3,4,5-Cl3 | — | CH2 | CH2 | CHNHCOC2H5 |
| 9-116 | 3,4,5-Cl3 | — | CH2 | CH2 | CHNHCOCH2CF3 |
| 9-117 | 3,4,5-Cl3 | — | CH2 | CH2 | CHNHCOPr-c |
| 9-118 | 3,4,5-Cl3 | — | CH2 | CH2 | CHNHCOCH2Pr-c |
| 9-119 | 3,4,5-Cl3 | — | CH2 | CH2 | CHNHCOCH2SMe |
| 9-120 | 3,4,5-Cl3 | — | CH2 | CH2 | CHNHCOCH2SOMe |
| 9-121 | 3,4,5-Cl3 | — | CH2 | CH2 | CHNHCOCH2SO2Me |
| 9-122 | 3,4,5-Cl3 | — | CH2 | CH2 | CHNHC(═O)NHCH3 |
| 9-123 | 3,4,5-Cl3 | — | CH2 | CH2 | CHNHC(═O)NHC2H5 |
| 9-124 | 3,4,5-Cl3 | — | CH2 | CH2 | CHNHC(═O)NHCH2CCH |
| 9-125 | 3,4,5-Cl3 | — | CH2 | CH2 | CHNHCO2C4H9-t |
| 9-126 | 3,4,5-Cl3 | — | CH2 | CH2 | CHNHCO2CH3 |
| 9-127 | 3,4,5-Cl3 | — | CH2 | CH2 | CHNHCO2Ph |
| 9-128 | 3,4,5-Cl3 | — | CH2 | CH2 | CHOC(═O)CH3 |
| 9-129 | 3,4,5-Cl3 | — | CH2 | CH2 | CHN3 |
| 9-130 | 3,4,5-Cl3 | — | CH2 | CH2 | CHNHC(═S)NHC2H5 |
| 9-131 | 3,4,5-Cl3 | — | CH2 | CH2 | CHNHCSC2H5 |
| 9-132 | 3,4,5-Cl3 | — | CH2 | CHNHCOC2H5 | CH2 |
| 9-133 | 3,4,5-Cl3 | — | CH2 | CHNHCO2C4H9-t | CH2 |
| 9-134 | 3,4,5-Cl3 | — | CH2 | C═O | CH2 |
| 9-135 | 3,4,5-Cl3 | — | CH2 | C═NOH | CH2 |
| 9-136 | 3,4,5-Cl3 | — | O | N | CHNHCO2C4H9-t |
| 9-137 | 3,4,5-Cl3 | — | NCH3 | N | CHNHCO2C4H9-t |
| 9-138 | 3,4,5-Cl3 | — | SO2 | N | CHNHCO2C4H9-t |
| 9-139 | 3,4,5-Cl3 | — | CH | CH | CHNHCOCH3 |
| 9-140 | 3,4,5-Cl3 | — | CH | CH | CHNHCOC2H5 |
| 9-141 | 3,4,5-Cl3 | — | CH | CH | CHNHCOCH2CF3 |
| 9-142 | 3,4,5-Cl3 | — | CH | CH | CHNHCOPr-c |
| 9-143 | 3,4,5-Cl3 | — | CH | CH | CHNHCOCH2SMe |
| 9-144 | 3,4,5-Cl3 | — | CH | CH | CHNHCOCH2SOMe |
| 9-145 | 3,4,5-Cl3 | — | CH | CH | CHNHCOCH2SO2Me |
| 9-146 | 3,4,5-Cl3 | — | CH | CH | CHNHC(═O)NHC2H5 |
| 9-147 | 3,4,5-Cl3 | — | CH | CH | CHNHCO2C4H9-t |
| 9-148 | 3,4,5-Cl3 | — | CH | CH | CHOC(═O)CH3 |
| 9-149 | 3,4,5-Cl3 | — | CH | CH | CHN3 |
| 9-150 | 3,4,5-Cl3 | — | CH2 | CH2 | NNHCOCH3 |
| 9-151 | 3,4,5-Cl3 | — | CH2 | CH2 | NNHCOC2H5 |
| 9-152 | 3,4,5-Cl3 | — | CH2 | CH2 | NNHCOCH2CF3 |
| 9-153 | 3,4,5-Cl3 | — | CH2 | CH2 | NNHCOPr-c |
| 9-154 | 3,4,5-Cl3 | — | CH2 | CH2 | NNHCOCH2SMe |
| 9-155 | 3,4,5-Cl3 | — | CH2 | CH2 | NNHCOCH2SOMe |
| 9-156 | 3,4,5-Cl3 | — | CH2 | CH2 | NNHCOCH2SO2Me |
| 9-157 | 3,4,5-Cl3 | — | CH2 | CH2 | NNHC(═O)NHC2H5 |
| 9-158 | 3,4,5-Cl3 | — | CH2 | CH2 | NNHCO2C4H9-t |
| 9-159 | 3,4,5-Cl3 | — | CH2 | CHCH3 | NNHCO2C4H9-t |
| 9-160 | 3,4,5-Cl3 | — | CH2 | C(CH3)2 | NNHCO2C4H9-t |
| 9-161 | 3,4,5-Cl3 | — | CH2 | CHCl | NNHCO2C4H9-t |
| 9-162 | 3,4,5-Cl3 | — | CH2 | CHF | NNHCO2C4H9-t |
| 9-163 | 3,5-(CF3)2 | — | CH2 | CH2 | CHNHCOCH3 |
| 9-164 | 3,5-(CF3)2 | — | CH2 | CH2 | CHNHCOC2H5 |
| 9-165 | 3,5-(CF3)2 | — | CH2 | CH2 | CHNHCOCH2CF3 |
| 9-166 | 3,5-(CF3)2 | — | CH2 | CH2 | CHNHCOPr-c |
| 9-167 | 3,5-(CF3)2 | — | CH2 | CH2 | CHNHCOCH2SO2Me |
| 9-168 | 3,5-(CF3)2 | — | CH2 | CH2 | CHNHC(═O)NHC2H5 |
| 9-169 | 3,5-(CF3)2 | — | CH2 | CH2 | CHNHCO2C4H9-t |
| 9-170 | 3-CF3 | — | CH2 | CH2 | CHNHCOCH3 |
| 9-171 | 3-CF3 | — | CH2 | CH2 | CHNHCOC2H5 |
| 9-172 | 3-CF3 | — | CH2 | CH2 | CHNHCOCH2CF3 |
| 9-173 | 3-CF3 | — | CH2 | CH2 | CHNHCOPr-c |
| 9-174 | 3-CF3 | — | CH2 | CH2 | CHNHCOCH2SO2Me |
| 9-175 | 3-CF3 | — | CH2 | CH2 | CHNHC(═O)NHC2H5 |
| 9-176 | 3-CF3 | — | CH2 | CH2 | CHNHCO2C4H9-t |
| 9-177 | 3,5-Cl2 | CH2 | CH2 | O | C═O |
| 9-178 | 3,5-Cl2 | CH2 | C═O | O | CH2 |
| 9-179 | 3,5-Cl2 | CH | N | CH | O |
| 9-180 | 3,5-Cl2 | CH | N | CH | S |
| 9-181 | 3,5-Cl2 | CH | N | CH | NH |
| 9-182 | 3,5-Cl2 | CH2 | CH2 | CH2 | C═O |
| 9-183 | 3,5-Cl2 | CH2 | CH2 | C═O | CH2 |
| 9-184 | 3,5-Cl2 | CH2 | C═O | CH2 | CH2 |
| 9-185 | 3,5-Cl2 | CH2 | CH2 | C═O | NCOCH3 |
| 9-186 | 3,5-Cl2 | CH2 | CH2 | C═O | NCOCH2CF3 |
| 9-187 | 3,5-Cl2 | CH2 | CH2 | CH2 | NCOCH3 |
| 9-188 | 3,5-Cl2 | CH2 | CH2 | CH2 | NCOC2H5 |
| 9-189 | 3,5-Cl2 | CH2 | CH2 | CH2 | NCOCH2CF3 |
| 9-190 | 3,5-Cl2 | CH2 | CH2 | CH2 | N(C═O)NCH3 |
| 9-191 | 3,5-Cl2 | CH2 | CH2 | CH2 | N(C═O)NC2H5 |
| 9-192 | 3,5-Cl2 | CH2 | CH2 | CH2 | N(C═O)NCH2CCH |
| 9-193 | 3,5-Cl2 | CH2 | CH2 | NCOCH3 | CH2 |
| 9-194 | 3,5-Cl2 | CH2 | CH2 | NCOC2H5 | CH2 |
| 9-195 | 3,5-Cl2 | CH2 | CH2 | NCOCH2CF3 | CH2 |
| 9-196 | 3,5-Cl2 | CH2 | CH2 | CH2 | C═NOH |
| 9-197 | 3,5-Cl2 | CH2 | CH2 | CH2 | C═NOCH3 |
| 9-198 | 3,5-Cl2 | CH2 | CH2 | CH2 | C═NOCH2CF3 |
| 9-199 | 3,5-Cl2 | CH2 | CH2 | CH2 | CHNHCOCH3 |
| 9-200 | 3,5-Cl2 | CH2 | CH2 | CH2 | CHNHCOC2H5 |
| 9-201 | 3,5-Cl2 | CH2 | CH2 | CH2 | CHNHCOCH2CF3 |
| 9-202 | 3,5-Cl2 | CH2 | CH2 | CH2 | CHNHCOPr-c |
| 9-203 | 3,5-Cl2 | CH2 | CH2 | CH2 | CHNHCOCH2Pr-c |
| 9-204 | 3,5-Cl2 | CH2 | CH2 | CH2 | CHNHCOCH2SMe |
| 9-205 | 3,5-Cl2 | CH2 | CH2 | CH2 | CHNHCOCH2SOMe |
| 9-206 | 3,5-Cl2 | CH2 | CH2 | CH2 | CHNHCOCH2SO2Me |
| 9-207 | 3,5-Cl2 | CH2 | CH2 | CH2 | CHNHC(═O)NHCH3 |
| 9-208 | 3,5-Cl2 | CH2 | CH2 | CH2 | CHNHC(═O)NHC2H5 |
| 9-209 | 3,5-Cl2 | CH2 | CH2 | CH2 | CHNHC(═O)NHCH2CCH |
| 9-210 | 3,5-Cl2 | CH2 | CH2 | CH2 | CHNHCO2C4H9-t |
| 9-211 | 3,5-Cl2 | CH2 | CH2 | CH2 | CHNHCO2CH3 |
| 9-212 | 3,5-Cl2 | CH2 | CH2 | CH2 | CHNHCO2Ph |
| 9-213 | 3,5-Cl2 | CH2 | CH2 | CH2 | CHOC(═O)CH3 |
| 9-214 | 3,5-Cl2 | CH2 | CH2 | CH2 | CHN3 |
| 9-215 | 3,5-Cl2 | CH2 | CH2 | CH2 | CHNHC(═S)NHC2H5 |
| 9-216 | 3,5-Cl2 | CH2 | CH2 | CH2 | CHNHCSC2H5 |
| 9-217 | 3,5-Cl2 | CH2 | CH2 | CHNHCOC2H5 | CH2 |
| 9-218 | 3,5-Cl2 | CH2 | CH2 | CHNHCO2C4H9-t | CH2 |
| 9-219 | 3,5-Cl2 | CH2 | CH2 | C═O | CH2 |
| 9-220 | 3,5-Cl2 | CH2 | CH2 | C═NOH | CH2 |
| 9-221 | 3,5-Cl2 | CH | CH | CH | CNHCOCH3 |
| 9-222 | 3,5-Cl2 | CH | CH | CH | CNHCOC2H5 |
| 9-223 | 3,5-Cl2 | CH | CH | CH | CNHCOCH2CF3 |
| 9-224 | 3,5-Cl2 | CH | CH | CH | CNHCOPr-c |
| 9-225 | 3,5-Cl2 | CH | CH | CH | CNHCOCH2SMe |
| 9-226 | 3,5-Cl2 | CH | CH | CH | CNHCOCH2SOMe |
| 9-227 | 3,5-Cl2 | CH | CH | CH | CNHCOCH2SO2Me |
| 9-228 | 3,5-Cl2 | CH | CH | CH | CNHC(═O)NHC2H5 |
| 9-229 | 3,5-Cl2 | CH | CH | CH | CNHCO2C4H9-t |
| 9-230 | 3,5-Cl2 | CH | CH | CH | COC(═O)CH3 |
| 9-231 | 3,5-Cl2 | CH | CH | CH | CN3 |
| 9-232 | 3,5-Cl2 | CH2 | CH2 | CH2 | NNHCOCH3 |
| 9-233 | 3,5-Cl2 | CH2 | CH2 | CH2 | NNHCOC2H5 |
| 9-234 | 3,5-Cl2 | CH2 | CH2 | CH2 | NNHCOCH2CF3 |
| 9-235 | 3,5-Cl2 | CH2 | CH2 | CH2 | NNHCOPr-c |
| 9-236 | 3,5-Cl2 | CH2 | CH2 | CH2 | NNHCOCH2SMe |
| 9-237 | 3,5-Cl2 | CH2 | CH2 | CH2 | NNHCOCH2SOMe |
| 9-238 | 3,5-Cl2 | CH2 | CH2 | CH2 | NNHCOCH2SO2Me |
| 9-239 | 3,5-Cl2 | CH2 | CH2 | CH2 | NNHC(═O)NHC2H5 |
| 9-240 | 3,5-Cl2 | CH2 | CH2 | CH2 | NNHCO2C4H9-t |
| 9-241 | 3,5-Cl2 | CH2 | CH2 | CHCH3 | NNHCO2C4H9-t |
| 9-242 | 3,5-Cl2 | CH2 | CH2 | C(CH3)2 | NNHCO2C4H9-t |
| 9-243 | 3,5-Cl2 | CH2 | CH2 | CHCl | NNHCO2C4H9-t |
| 9-244 | 3,5-Cl2 | CH2 | CH2 | CHF | NNHCO2C4H9-t |
| 9-245 | 3,4,5-Cl3 | CH2 | CH2 | O | C═O |
| 9-246 | 3,4,5-Cl3 | CH2 | CH2 | O | CH2 |
| 9-247 | 3,4,5-Cl3 | CH2 | CH2 | CH2 | O |
| 9-248 | 3,4,5-Cl3 | CH2 | C═O | O | CH2 |
| 9-249 | 3,4,5-Cl3 | CH2 | CH2 | CH2 | C═O |
| 9-250 | 3,4,5-Cl3 | CH2 | CH | CH | C═O |
| 9-251 | 3,4,5-Cl3 | CH2 | CH2 | C═O | CH2 |
| 9-252 | 3,4,5-Cl3 | CH2 | C═O | CH2 | CH2 |
| 9-253 | 3,4,5-Cl3 | CH2 | CH2 | C═O | NCOCH3 |
| 9-254 | 3,4,5-Cl3 | CH2 | CH2 | C═O | NCOCH2CF3 |
| 9-255 | 3,4,5-Cl3 | CH2 | CH2 | CH2 | NCOCH3 |
| 9-256 | 3,4,5-Cl3 | CH2 | CH2 | CH2 | NCOC2H5 |
| 9-257 | 3,4,5-Cl3 | CH2 | CH2 | CH2 | NCOCH2CF3 |
| 9-258 | 3,4,5-Cl3 | CH2 | CH2 | CH2 | N(C═O)NCH3 |
| 9-259 | 3,4,5-Cl3 | CH2 | CH2 | CH2 | N(C═O)NC2H5 |
| 9-260 | 3,4,5-Cl3 | CH2 | CH2 | CH2 | N(C═O)NCH2CCH |
| 9-261 | 3,4,5-Cl3 | CH2 | CH2 | NCOCH3 | CH2 |
| 9-262 | 3,4,5-Cl3 | CH2 | CH2 | NCOC2H5 | CH2 |
| 9-263 | 3,4,5-Cl3 | CH2 | CH2 | NCOCH2CF3 | CH2 |
| 9-264 | 3,4,5-Cl3 | CH2 | CH2 | CH2 | C═NOH |
| 9-265 | 3,4,5-Cl3 | CH2 | CH2 | CH2 | C═NOCH3 |
| 9-266 | 3,4,5-Cl3 | CH2 | CH2 | CH2 | C═NOCH2CF3 |
| 9-267 | 3,4,5-Cl3 | CH2 | CH2 | CH2 | CHNHCOCH3 |
| 9-268 | 3,4,5-Cl3 | CH2 | CH2 | CH2 | CHNHCOC2H5 |
| 9-269 | 3,4,5-Cl3 | CH2 | CH2 | CH2 | CHNHCOCH2CF3 |
| 9-270 | 3,4,5-Cl3 | CH2 | CH2 | CH2 | CHNHCOPr-c |
| 9-271 | 3,4,5-Cl3 | CH2 | CH2 | CH2 | CHNHCOCH2Pr-c |
| 9-272 | 3,4,5-Cl3 | CH2 | CH2 | CH2 | CHNHCOCH2SMe |
| 9-273 | 3,4,5-Cl3 | CH2 | CH2 | CH2 | CHNHCOCH2SOMe |
| 9-274 | 3,4,5-Cl3 | CH2 | CH2 | CH2 | CHNHCOCH2SO2Me |
| 9-275 | 3,4,5-Cl3 | CH2 | CH2 | CH2 | CHNHC(═O)NHCH3 |
| 9-276 | 3,4,5-Cl3 | CH2 | CH2 | CH2 | CHNHC(═O)NHC2H5 |
| 9-277 | 3,4,5-Cl3 | CH2 | CH2 | CH2 | CHNHC(═O)NHCH2CCH |
| 9-278 | 3,4,5-Cl3 | CH2 | CH2 | CH2 | CHNHCO2C4H9-t |
| 9-279 | 3,4,5-Cl3 | CH2 | CH2 | CH2 | CHNHCO2CH3 |
| 9-280 | 3,4,5-Cl3 | CH2 | CH2 | CH2 | CHNHCO2Ph |
| 9-281 | 3,4,5-Cl3 | CH2 | CH2 | CH2 | CHOC(═O)CH3 |
| 9-282 | 3,4,5-Cl3 | CH2 | CH2 | CH2 | CHN3 |
| 9-283 | 3,4,5-Cl3 | CH2 | CH2 | CH2 | CHNHC(═S)NHC2H5 |
| 9-284 | 3,4,5-Cl3 | CH2 | CH2 | CH2 | CHNHCSC2H5 |
| 9-285 | 3,4,5-Cl3 | CH2 | CH2 | CHNHCOC2H5 | CH2 |
| 9-286 | 3,4,5-Cl3 | CH2 | CH2 | CHNHCO2C4H9-t | CH2 |
| 9-287 | 3,4,5-Cl3 | CH2 | CH2 | C═O | CH2 |
| 9-288 | 3,4,5-Cl3 | CH2 | CH2 | C═NOH | CH2 |
| 9-289 | 3,4,5-Cl3 | CH | CH | CH | CNHCOCH3 |
| 9-290 | 3,4,5-Cl3 | CH | CH | CH | CNHCOC2H5 |
| 9-291 | 3,4,5-Cl3 | CH | CH | CH | CNHCOCH2CF3 |
| 9-292 | 3,4,5-Cl3 | CH | CH | CH | CNHCOPr-c |
| 9-293 | 3,4,5-Cl3 | CH | CH | CH | CNHCOCH2SMe |
| 9-294 | 3,4,5-Cl3 | CH | CH | CH | CNHCOCH2SOMe |
| 9-295 | 3,4,5-Cl3 | CH | CH | CH | CNHCOCH2SO2Me |
| 9-296 | 3,4,5-Cl3 | CH | CH | CH | CNHC(═O)NHC2H5 |
| 9-297 | 3,4,5-Cl3 | CH | CH | CH | CNHCO2C4H9-t |
| 9-298 | 3,4,5-Cl3 | CH | CH | CH | COC(═O)CH3 |
| 9-299 | 3,4,5-Cl3 | CH | CH | CH | CN3 |
| 9-300 | 3,4,5-Cl3 | CH2 | CH2 | CH2 | NNHCOCH3 |
| 9-301 | 3,4,5-Cl3 | CH2 | CH2 | CH2 | NNHCOC2H5 |
| 9-302 | 3,4,5-Cl3 | CH2 | CH2 | CH2 | NNHCOCH2CF3 |
| 9-303 | 3,4,5-Cl3 | CH2 | CH2 | CH2 | NNHCOPr-c |
| 9-304 | 3,4,5-Cl3 | CH2 | CH2 | CH2 | NNHCOCH2SMe |
| 9-305 | 3,4,5-Cl3 | CH2 | CH2 | CH2 | NNHCOCH2SOMe |
| 9-306 | 3,4,5-Cl3 | CH2 | CH2 | CH2 | NNHCOCH2SO2Me |
| 9-307 | 3,4,5-Cl3 | CH2 | CH2 | CH2 | NNHC(═O)NHC2H5 |
| 9-308 | 3,4,5-Cl3 | CH2 | CH2 | CH2 | NNHCO2C4H9-t |
| 9-309 | 3,4,5-Cl3 | CH2 | CH2 | CHCH3 | NNHCO2C4H9-t |
| 9-310 | 3,4,5-Cl3 | CH2 | CH2 | C(CH3)2 | NNHCO2C4H9-t |
| 9-311 | 3,4,5-Cl3 | CH2 | CH2 | CHCl | NNHCO2C4H9-t |
| 9-312 | 3,4,5-Cl3 | CH2 | CH2 | CHF | NNHCO2C4H9-t |
| 9-313 | 3,5-(CF3)2 | CH2 | CH2 | CH2 | CHNHCOCH3 |
| 9-314 | 3,5-(CF3)2 | CH2 | CH2 | CH2 | CHNHCOC2H5 |
| 9-315 | 3,5-(CF3)2 | CH2 | CH2 | CH2 | CHNHCOCH2CF3 |
| 9-316 | 3,5-(CF3)2 | CH2 | CH2 | CH2 | CHNHCOPr-c |
| 9-317 | 3,5-(CF3)2 | CH2 | CH2 | CH2 | CHNHCOCH2SO2Me |
| 9-318 | 3,5-(CF3)2 | CH2 | CH2 | CH2 | CHNHC(═O)NHC2H5 |
| 9-319 | 3,5-(CF3)2 | CH2 | CH2 | CH2 | CHNHCO2C4H9-t |
| 9-320 | 3-CF3 | CH2 | CH2 | CH2 | CHNHCOCH3 |
| 9-321 | 3-CF3 | CH2 | CH2 | CH2 | CHNHCOC2H5 |
| 9-322 | 3-CF3 | CH2 | CH2 | CH2 | CHNHCOCH2CF3 |
| 9-323 | 3-CF3 | CH2 | CH2 | CH2 | CHNHCOPr-c |
| 9-324 | 3-CF3 | CH2 | CH2 | CH2 | CHNHCOCH2SO2Me |
| 9-325 | 3-CF3 | CH2 | CH2 | CH2 | CHNHC(═O)NHC2H5 |
| 9-326 | 3-CF3 | CH2 | CH2 | CH2 | CHNHCO2C4H9-t |

| No. | 1H-NMR |
|---|---|
| 1-2 | 1H-NMR (CDCl3) δ: 3.94 (2H, m), 5.36 (2H, s), 7.44-7.51 (3H, m), 7.78-8.04 (3H, m). |
| 1-10 | 1H-NMR (CDCl3) δ: 3.81 (1H, d), 4.25 (1H, d), 5.37 (2H, s), 7.80-7.83 (1H, m), 7.87-7.91 (1H, m), 7.99-8.02 (2H, m), 8.07-8.10 (2H, m) |
| 1-38 | 1H-NMR (CDCl3) δ: 3.74 (1H, d), 4.13 (1H, d), 5.36 (2H, s), 7.63-7.66 (2H, m), 7.78-7.79 (1H, m), 7.85-7.88 (1H, m), 7.98-8.01 (1H, m) |
| 1-44 | 1H-NMR (CDCl3) δ: 3.76 (1H, d), 4.19 (1H, d), 5.36 (2H, s), 7.80-7.86 (3H, m), 7.96-8.00 (2H, m) |
| 1-50 | 1H-NMR (CDCl3) δ: 3.75 (1H, d), 4.14 (1H, d), 4.50 (2H, s), 6.48-6.49 (1H, m), 7.44-7.44 (1H, m), 7.51-7.52 (2H, m), 7.71-7.73 (1H, m), |
| | 7.86-7.89 (1H, m), 7.92-7.95 (1H, m) |
| 1-52 | 1H-NMR (CDCl3) δ: 3.24 (3H, s), 3.72-4.16 (2H, m), 4.42 (2H, s), 7.43 (1H, m), 7.52 (2H, m), 7.67 (1H, m), 7.88 (2H, m). |
| 1-54 | 1H-NMR (CDCl3) δ: 1.30 (3H, t), 3.69 (2H, q), 3.94 (2H, m), 4.42 (2H, s), 7.43 (1H, m), 7.52 (2H, m), 7.66 (1H, m), 7.88 (2H, m). |
| 1-56 | 1H-NMR (CDCl3) δ: 3.72-3.78 (1H, m), 4.11-4.16 (1H, m), 4.24 (2H, q, J = 9.0 Hz), 4.60 (1H, s), 7.42-7.46 (1H, m), 7.51 (2H, s), |
| | 7.72 (1H, d, J = 7.9 Hz), 7.88 (1H, s), 7.95 (1H, d, J = 7.9 Hz). |
| 1-58 | 1H-NMR (CDCl3) δ: 3.74 (1H, d), 4.12 (1H, d), 4.52 (2H, s), 4.93 (2H, s), 7.20-7.22 (1H, m), 7.31-7.34 (1H, m), 7.43-7.43 (1H, m), |
| | 7.51-7.51 (2H, m), 7.65-7.70 (2H, m), 7.80-7.83 (1H, m), 7.92-7.95 (1H, m), 8.55-8.57 (1H, m) |
| 1-60 | 1H-NMR (CDCl3) δ: 0.97 (3H, t), 1.68-1.78 (2H, m), 3.60 (2H, t), 3.94 (2H, m), 4.41 (2H, s), 7.44 (1H, m), 7.52 (2H, m), 7.66 (1H, m), |
| | 7.88 (2H, m). |
| 1-61 | 1H-NMR (CDCl3) δ: 1.31 (6H, d), 3.94 (2H, m), 4.37 (2H, s), 4.61-4.75 (1H, m), 7.44 (1H, m), 7.52 (2H, m), 7.64 (1H, m), 7.89 (2H, m). |
| 1-62 | 1H-NMR (CDCl3) δ: 0.92 (4H, m), 2.92-2.99 (1H, m), 3.93 (2H, m), 4.38 (2H, s), 7.43 (1H, m), 7.51 (2H, m), 7.66 (1H, m), 7.86 (2H, m). |
| 1-64 | 1H-NMR (CDCl3) δ: 0.97 (6H, d), 2.03-2.12 (1H, m), 3.44 (2H, d), 3.72-4.16 (2H, m), 4.44 (2H, s), 7.44 (1H, m), 7.52 (2H, m), 7.67 |
| | (1H, m), 7.90 (2H, m). |
| 1-67 | 1H-NMR (CDCl3) δ: 3.94 (2H, m), 4.25 (2H, d), 4.42 (2H, s), 5.25 (2H, m), 5.87 (1H, m), 7.43 (1H, m), 7.52 (2H, m), 7.68 (1H, m), |
| | 7.90 (2H, m). |
| 1-69 | 1H-NMR (CDCl3) δ: 3.35 (3H, s), 3.68 (2H, t), 3.76 (2H, t), 3.94 (2H, m), 4.57 (2H, s), 7.43 (1H, m), 7.52 (2H, m), 7.68 (1H, m), |
| | 7.80-7.94 (2H, m). |
| 1-79 | 1H-NMR (CDCl3) δ: 3.93 (2H, m), 4.32 (2H, s), 4.81 (2H, s), 7.29-7.50 (8H, m), 7.68-7.95 (3H, s) |
| 1-80 | 1H-NMR (acetone-d6) δ: 4.32 (1H, d), 4.48 (1H, d), 4.75 (2H, d), 5.02 (2H, d), 7.35 (1H, t), 7.65-7.61 (1H, m), 7.68 (2H, s), 7.82 |
| | (1H, d), 7.92 (1H, d), 8.01 (1H, s), 8.71 (2H, d) |
| 1-81 | 1H-NMR (CDCl3) δ: 3.77 (1H, d), 4.15 (1H), 4.91 (2H, s), 7.22 (1H, t), 7.42-7.48 (3H, m), 7.53 (2H, s), 7.73 (1H, d), 7.87 (2H, d), |
| | 7.93 (1H, s), 7.99 (1H, d) |
| 1-89 | 1H-NMR (acetone-d6) δ: 4.32 (1H, d), 4.48 (1H, d), 4.75 (2H, d), 5.02 (2H, d), 7.35 (1H, t), 7.65-7.61 (1H, m), 7.68 (2H, s), 7.82 |
| | (1H, d), 7.92 (1H, d), 8.01 (1H, s), 8.71 (2H, d) |
| 1-114 | 1H-NMR (CDCl3) δ: 3.23 (3H, s), 3.93 (2H, m), 5.36 (2H, s), 7.64 (6H, m). |
| 1-115 | 1H-NMR (CDCl3) δ: 3.92 (2H, m), 4.76 (2H, d), 5.24 (2H, d), 7.19-7.72 (7H, m), 7.93 (1H, s), 8.20 (1H, s), 8.54 (1H, d). |
| 1-116 | 1H-NMR (CDCl3) δ: 3.95 (2H, m), 5.38 (2H, s), 7.44-7.61 (4H, m), 8.03 (1H, s), 8.20 (1H, d). |
| 1-117 | 1H-NMR (CDCl3) δ: 3.98 (2H, m), 7.48 (3H, m), 8.19 (3H, m). |
| 1-120 | 1H-NMR (CDCl3) δ: 3.21 (3H, s), 4.00 (2H, m), 7.44-7.51 (3H, m), 7.91 (1H, m), 8.06-8.11 (2H, m). |
| 1-121 | 1H-NMR (CDCl3) δ: 3.57 (2H, s), 3.68 (1H, d), 4.06 (1H, t), 6.93 (1H, d), 7.40-7.50 (4H, m), 7.64 (1H, s), 9.02 (1H, s). |
| 1-122 | 1H-NMR (CDCl3) δ: 2.07 (3H, s), 3.71-3.73 (1H, m), 4.01-4.07 (1H, m), 4.28 (1H, s), 6.93 (1H, d), 7.43 (1H, d, J = 0.9 Hz), |
| | 7.53-7.59 (3H, m), 7.71 (1H, d), 8.09 (1H, s). |
| 1-125 | 1H-NMR (CDCl3) δ: 3.72 (1H, d), 4.10-4.17 (3H, m), 7.15 (1H, s), 7.44 (1H, s), 7.49-7.53 (2H, m), 7.61-7.64 (2H, m), 8.12-8.14 |
| | (1H, m) |
| 1-126 | 1H-NMR (CDCl3) δ: 2.61 (3H, s), 3.74 (1H, d), 4.13 (1H, d), 4.24 (2H, s), 7.43 (1H, s), 7.51 (2H, s), 7.61-7.64 (2H, m), 7.83 |
| | (1H, s), 8.11 (1H, d) |
| 1-127 | 1H-NMR (CDCl3) δ: 2.51 (3H, s), 3.23 (3H, s), 3.72 (1H, d), 4.11 (1H, d), 4.23 (2H, s), 7.43-7.44 (1H, m), 7.51 (2H, s), 7.59-7.60 |
| | (2H, m), 8.10 (1H, d) |
| 1-129 | 1H-NMR (CDCl3) δ: 3.83 (1H, d), 4.20 (1H, d), 7.44-7.44 (1H, m), 7.52-7.53 (2H, m), 7.99-7.99 (1H, m), 8.09-8.12 (1H, m), 8.20-8.24 |
| | (1H, m), 8.47-8.50 (1H, m), 11.25 (1H, br s) |
| 1-131 | 1H-NMR (CDCl3) δ: 3.72 (1H, d), 4.11 (1H, d), 5.11 (4H, s), 7.27-7.30 (1H, m), 7.41-7.41 (1H, m), 7.51-7.57 (4H, m) |
| 1-132 | 1H-NMR (CDCl3) δ: 3.72 (1H, d), 4.11 (1H, d), 5.12 (4H, s), 7.28-7.30 (1H, m), 7.53-7.57 (2H, m), 7.63-7.66 (2H, m) |
| 1-134 | 1H-NMR (CDCl3) δ: 3.64 (1H, d), 4.03 (1H, d), 6.02 (2H, s), 6.83-6.85 (1H, m), 7.01-7.03 (1H, m), 7.41-7.43 (1H, m), 7.49-7.50 (3H, m) |
| 1-135 | 1H-NMR (CDCl3) δ: 3.83 (1H, d), 4.24 (1H, d), 6.04 (2H, s), 6.86 (1H, s), 7.16 (1H, s), 7.42-7.43 (1H, m), 7.48-7.49 (2H, m) |
| 1-136 | 1H-NMR (CDCl3) δ: 3.23 (2H, t), 3.66 (1H, d), 4.06 (1H, d), 4.63 (2H, t), 6.78-6.81 (1H, m), 7.33-7.36 (1H, m), 7.40-7.41 (1H, m), |
| | 7.51-7.51 (2H, m), 7.59-7.60 (1H, m) |
| 1-141 | 1.58 (bs, 1H), 3.05 (t, 2H), 3.57-3.69 (m, 3H), 4.04 (d, 1H), 6.56 (d, 1H), 7.23 (d, 1H), 7.40 (s, 1H), 7.47 (s, 1H), 7.51 (s, 2H) |
| 1-142 | 1H-NMR (CDCl3) δ: 2.81 (s, 3H), 2.98 (t, 2H), 3.43 (t, 2H), 3.63 (d, 1H), 4.03 (d, 1H), 6.37 (d, 1H), 7.26 (d, 1H), 7.40 (s, 1H), |
| | 7.44 (s, 1H), 7.52 (s, 2H) |
| 1-145 | 1H-NMR (CDCl3) δ: 2.25 (s, 3H), 3.23 (t, 2H), 3.68 (d, 1H), 4.02-4.16 (m, 3H), 7.34 (d, 1H), 7.42 (s, 1H), 7.51 (s, 2H), 7.63 (s, 1H), |
| | 8.23 (d, 1H) |
| 1-146 | 1H-NMR (CDCl3) δ: 3.30 (t, 2H), 3.69 (d, 1H), 4.08 (d, 1H), 4.34 (t, 2H), 7.39-7.47 (m, 2H), 7.51 (s, 2H), 7.71 (s, 1H), 8.24 (d, 1H) |
| 1-149 | 1H-NMR (CDCl3) δ: 3.87 (2H, m), 3.91 (2H, s), 3.95 (4H, s), 7.21-7.51 (11H, m). |
| 1-150 | 1H-NMR (CDCl3) δ: 3.89 (2H, m), 4.04 (4H, s), 4.08 (2H, s), 7.19-7.70 (9H, m), 8.59 (1H, d, J = 4.9 Hz). |
| 1-151 | 1H-NMR (CDCl3) δ: 2.18 (3H, s), 3.71 (1H, d), 4.10 (1H, d), 4.82-4.85 (4H, m), 7.32-7.39 (1H, m), 7.43-7.43 (1H, m), 7.52-7.65 (4H, m) |
| 1-155 | 1H-NMR (CDCl3) δ: 3.79 (d, 1H), 4.18 (d, 1H), 6.59 (s, 1H), 7.37-7.85 (m, 7H), 8.34 (bs, 1H) |
| 1-157 | 1H-NMR (CDCl3) δ: 3.77 (d, 1H), 4.17 (d, 1H), 6.59 (d, 1H), 7.42 (s, 1H), 7.54 (s, 2H), 7.61-7.70 (m, 2H), 7.82 (s, 1H), 8.19 (d, 1H) |
| 1-163 | 1H-NMR (CDCl3) δ: 3.85 (1H, d), 4.23 (1H, d), 7.43-7.50 (2H, m), 7.55 (2H, s), 7.87 (1H, d), 8.05-8.14 (2H, m), 8.18 (1H, d), |
| | 9.00-8.93 (1H, m) |
| 1-164 | 1H-NMR (CDCl3) δ: 3.83 (d, 1H), 4.23 (d, 1H), 7.41-7.59 (m, 4H), 7.95 (s, 1H), 8.13-8.22 (m, 3H), 8.95-9.00 (m, 1H) |
| 1-165 | 1H-NMR (CDCl3) δ: 1.86-2.00 (2H, m), 2.68-2.83 (2H, m), 3.24-3.40 (2H, m), 3.62 (1H, d), 3.95 (1H, s), 4.02 (1H, d), 6.73-6.86 |
| | (2H, m), 6.96 (1H, d), 7.41 (1H, s), 7.49 (2H, s) |
| 1-169 | 1H-NMR (CDCl3) δ: 1.55 (bs, 1H), 1.87-1.98 (m, 2H), 2.75 (t, 2H), 3.35 (t, 2H), 3.61 (d, 1H), 4.02 (d, 1H), 6.43 (d, 1H), 7.20-7.29 |
| | (m, 2H), 7.40 (s, 1H), 7.51 (s, 2H) |
| 1-170 | 1H-NMR (CDCl3) δ: 1.90-2.02 (m, 2H), 2.75 (t, 2H), 2.94 (s, 3H), 3.31 (t, 2H), 3.62 (d, 1H), 4.03 (d, 1H), 6.51 (d, 1H), 7.23-7.33 |
| | (m, 2H), 7.39 (s, 1H), 7.52 (s, 2H) |
| 1-171 | 1H-NMR (CDCl3) δ: 1.90-2.02 (m, 2H), 2.26 (s, 3H), 2.77 (t, 2H), 3.68 (d, 1H), 3.77 (t, 2H), 4.07 (d, 1H), 7.37-7.56 (m, 6H) |
| 1-175 | 1H-NMR (CDCl3) δ: 3.87 (1H, d), 4.25 (1H, d), 7.44-7.44 (1H, m), 7.55-7.55 (2H, m), 8.14-8.16 (2H, m), 8.29-8.32 (1H, m), 8.90 |
| | (2H, s). |
| 1-176 | 1H-NMR (CDCl3) δ: 3.39-3.46 (4H, m), 3.60 (1H, d), 4.00 (1H, d), 6.41-6.43 (1H, m), 6.74-6.77 (1H, m), 6.88-6.89 (1H, m), 7.39-7.40 |
| | (1H, m), 7.50-7.50 (2H, m). |
| 1-182 | 1H-NMR (CDCl3) δ: 3.78 (1H, d), 4.17 (1H, d), 7.42-7.43 (1H, m), 7.54-7.54 (2H, m), 7.63-7.66 (1H, m), 7.85-7.87 (1H, m), 8.00-8.00 |
| | (1H, m), 8.16 (1H, s). |
| 1-189 | 1H-NMR (CDCl3) δ: 3.80 (1H, d), 4.16 (1H, d), 7.43-7.44 (1H, m), 7.53-7.53 (2H, m), 7.69-7.71 (1H, m), 7.83-7.86 (1H, m), 7.94-7.94 |
| | (1H, m), 8.19 (1H, s). |
| 1-209 | 1H-NMR (CDCl3) δ: 2.74-2.76 (2H, m), 3.18-3.20 (2H, m), 3.75 (1H, d), 4.13 (1H, d), 7.44-7.44 (1H, m), 7.51-7.52 (2H, m), 7.66-7.69 |
| | (1H, m), 7.76-7.81 (2H, m) |
| 1-211 | 1H-NMR (CDCl3) δ: 2.94-3.18 (5H, m), 3.66-3.77 (1H, m), 4.08-4.13 (1H, m), 7.42-7.43 (1H, m), 7.49-7.57 (3H, m), 7.63 (1H, s), 7.69 |
| | (1H, d, J = 8.1 Hz). |
| 1-213 | 1H-NMR (CDCl3) δ: 2.85-2.96 (2H, m), 3.00-3.12 (2H, m), 3.71 (1H, d, J = 17.2 Hz), 4.01 (3H, s), 4.10 (1H, d, J = 17.2 Hz), |
| | 7.45-7.40 (1H, m), 7.57-7.49 (3H, m), 7.62 (1H, s), 7.73 (1H, d, J = 8.1 Hz). |
| 1-217 | 1H-NMR (CDCl3) δ: 2.59-2.68 (m, 2H), 3.06-3.18 (m, 5H), 3.70 (d, 1H), 4.09 (d, 1H), 7.39-7.55 (m 4H), 7.60 (s, 1H), 7.70 (d, 1H) |
| 1-219 | 1H-NMR (CDCl3) δ: 2.71 (s, 6H), 2.86-2.95 (m, 2H), 3.03-3.15 (m, 2H), 3.70 (d, 1H), 4.10 (d, 1H), 7.39-7.55 (m, 4H), 7.62 (s, 1H), |
| | 7.77 (d, 1H) |
| 1-221 | 1H-NMR (CDCl3) δ: 2.70 (3H, s), 3.76 (1H, d, J = 17.0 Hz), 4.14 (1H, d, J = 17.4 Hz), 4.85 (2H, s), 7.44-7.45 (1H, m), |
| | 7.51-7.52 (2H, m), 7.81-7.83 (2H, m), 7.96-7.99 (1H, m) |
| 1-222 | 1H-NMR (CDCl3) δ: 2.11-2.22 (m, 2H), 2.69 (t, 2H), 3.00 (t, 2H), 3.71 (d, 1H), 4.10 (d, 1H), 7.41-7.59 (m, 5H), 8.08 (d, 1H) |
| 1-223 | 1H-NMR (CDCl3) δ: 1.56 (bs, 1H), 1.83-1.94 (m, 2H), 2.74-2.87 (m, 4H), 3.70 (d, 1H), 4.09 (d, 1H), 7.41-7.56 (m, 5H), 7.97 (d, 1H) |
| 1-224 | 1H-NMR (CDCl3) δ: 1.78-1.90 (m, 2H), 2.68-2.80 (m, 4H), 3.69 (d, 1H), 4.01 (s, 3H), 4.08 (d, 1H), 7.39-7.55 (m, 5H), 8.03 (d, 1H) |
| 1-232 | 1H-NMR (CDCl3) δ: 3.74 (1H, d), 4.12 (1H, d), 5.34 (2H, s), 7.44-7.44 (1H, m), 7.49-7.52 (2H, m), 7.71-7.73 (1H, m), 7.76-7.79 |
| | (1H, m), 7.92-7.95 (1H, m) |
| 1-235 | 1H-NMR (CDCl3) δ: 2.12-2.24 (3H, m), 3.60-3.68 (1H, m), 4.02-4.08 (1H, m), 4.73-4.82 (4H, m), 7.13-7.73 (9H, m), 8.44-8.61 (1H, m) |
| 1-240 | 1H-NMR (CDCl3) δ: 3.07 (t, 2H), 3.54-3.68 (m, 3H), 4.04 (d, 1H), 4.48 (s, 2H), 6.38 (d, 1H), 7.16-7.69 (m, 8H), 8.59 (d, 1H) |
| 1-241 | 1H-NMR (CDCl3) δ: 2.95-3.14 (4H, m), 3.70 (1H, d), 4.09 (1H, d), 5.36 (2H, s), 7.17-7.24 (1H, m), 7.39-7.45 (2H, m), 7.47-7.55 |
| | (3H, m), 7.60-7.77 (3H, m), 8.60 (1H, d) |
| 1-242 | 1H-NMR (CDCl3) δ: 2.39 (s, 3H), 2.73-2.85 (m, 2H), 3.14-3.23 (m, 2H), 3.72 (d, 1H), 4.11 (d, 1H), 7.43 (s, 1H), 7.52 (s, 2H), 7.59 |
| | (d, 1H), 7.63 (s, 1H), 7.78 (d, 1H), 8.70 (s, 1H) |
| 1-243 | 1H-NMR (CDCl3) δ: 2.61-2.74 (m,. 2H), 3.07-3.23 (m, 2H), 3.70 (d, 1H), 3.91 (q, 2H), 4.10 (d, 1H), 5.09 (bs, 1H), 7.42 (s, 1H), |
| | 7.46-7.57 (m, 2H), 7.61 (s, 1H), 7.68 (d, 1H) |
| 1-244 | 1H-NMR (CDCl3) δ: 2.93-3.13 (m, 4H), 3.70 (d, 1H), 4.09 (d, 1H), 4.54 (q, 2H), 7.43 (s, 1H), 7.51 (s, 2H), 7.56 (d, 1H), 7.63 (s, 1H), |
| | 7.72 (d, 1H) |
| 1-245 | 1H-NMR (CDCl3) δ: 3.74 (1H, d), 4.12 (1H, d), 5.34 (2H, s), 7.44-7.44 (1H, m), 7.49-7.52 (2H, m), 7.71-7.73 (1H, m), 7.76-7.79 |
| | (1H, m), 7.92-7.95 (1H, m) |
| 1-246 | 1H-NMR (CDCl3) δ: 3.33 (dd, 1H), 3.73 (d, 1H), 3.81 (dd, 1H), 4.12 (d, 1H), 4.59 (dd, 1H), 7.44 (s, 1H), 7.51 (s, 2H), 7.74 (d, 1H), |
| | 7.78 (s, 1H), 7.88 (d, 1H) |
| 1-247 | 1H-NMR (CDCl3) δ: 3.74 (d, 1H), 4.12 (d, 1H), 7.44 (s, 1H), 7.52 (s, 2H), 7.56 (d, 1H), 7.72 (d, 1H), 8.02 (s, 1H) |
| 1-248 | 1H-NMR (CDCl3) δ: 3.73-3.78 (3H, m), 4.13 (1H, d), 6.99-7.02 (2H, m), 7.29-7.32 (1H, m), 7.43-7.44 (1H, m), 7.51-7.52 (2H, m), |
| | 8.55 (1H, s). |
| 1-249 | 1H-NMR (CDCl3) δ: 1.57 (6H, s), 3.77 (1H, d), 4.14-4.19 (3H, m), 4.36-4.39 (1H, m), 7.16 (1H, s), 7.28-7.29 (1H, m), 7.43-7.43 |
| | (1H, m), 7.51-7.51 (2H, m), 8.03-8.06 (1H, m), 8.11-8.11 (1H, m) |
| 1-250 | 1H-NMR (CDCl3) δ: 3.04 (t, 2H), 3.54-3.64 (m, 2H), 3.72 (d, 1H), 4.10 (d, 1H), 6.18 (bs, 1H), 7.43 (s, 1H), 7.51 (s, 2H), 7.56 |
| | (d, 1H), 7.63 (s, 1H), 8.13 (d, 1H) |
| 1-251 | 1H-NMR (CDCl3) δ: 2.67 (t, 2H), 3.01 (t, 2H), 3.67 (d, 1H), 4.06 (d, 1H), 6.85 (d, 1H), 7.38-7.60 (m, 5H), 8.91 (bs, 1H) |
| 1-252 | 1H-NMR (CDCl3) δ: 1.81-1.93 (m, 2H), 2.72-2.85 (m, 4H), 3.68 (d, 1H), 4.08 (d, 1H), 4.56 (q, 2H), 7.40-7.56 (m, 5H), 8.00 (d, 1H) |
| 1-253 | 1H-NMR (CDCl3) δ: 3.63 (1H, d), 4.03 (1H, d), 4.24-4.31 (4H, m), 6.89-6.92 (1H, m), 7.13-7.21 (2H, m), 7.41-7.41 (1H, m), 7.48-7.52 |
| | (2H, m). |
| 1-255 | 1H-NMR (CDCl3) δ: 3.84 (1H, d), 4.22 (1H, d), 7.44 (1H, t), 7.54-7.54 (2H, m), 7.77 (1H, d), 8.00 (1H, d), 8.21 (1H, d Hz), 8.29-8.33 |
| | (2H, m). |
| 1-256 | 1H-NMR (CDCl3) δ: 3.86 (d, 1H), 4.25 (d, 1H), 7.45 (s, 1H), 7.53-7.60 (m, 3H), 8.16 (d, 1H), 8.24-8.31 (m, 2H), 8.84 (d, 1H) |
| 1-257 | 1H-NMR (CDCl3) δ: 3.83 (d, 1H), 4.21 (d, 1H), 7.32-7.49 (m, 2H), 7.54 (s, 2H), 7.74 (d, 1H), 8.03 (s, 1H), 8.15 (d, 1H), 8.56 |
| | (d, 1H), 8.79 (d, 1H) |
| 1-259 | 1H-NMR (CDCl3) δ: 2.78 (3H, s), 3.81 (1H, d), 4.18 (1H, d), 7.44-7.52 (3H, m), 7.98-7.98 (1H, m), 8.08-8.11 (1H, m), 8.27 (1H, s), |
| | 8.52 (1H, d) |
| 1-260 | 1H-NMR (CDCl3) δ: 3.80 (1H, d), 4.18 (1H), 5.56 (2H, s), 7.18-7.20 (1H, m), 7.33 (1H, d), 7.44-7.44 (1H, m), 7.52-7.52 (2H, m), |
| | 7.64-7.67 (1H, m), 7.94 (1H, s), 8.06-8.09 (1H, m), 8.23 (1H, s), 8.48 (1H, d), 8.56-8.58 (1H, m) |
| 1-269 | 1H-NMR (CDCl3) δ: 3.77 (1H, d), 4.16 (1H, d), 4.94 (2H, s), 7.45 (1H, s), 7.52 (2H, s), 7.64-7.82 (3H, m), 7.92-8.11 (4H, m). |
| 1-270 | 1H-NMR (CDCl3) δ: 3.77 (1H, d), 4.16 (1H, d), 4.94 (2H, s), 7.38-7.65 (5H, m), 7.75 (1H, d), 7.94-8.03 (2H, m), 8.09-8.20 (2H, m). |
| 1-271 | 1H-NMR (CDCl3) δ: 3.76 (d, 1H), 3.94 (td, 2H), 4.14 (d, 1H), 4.59 (s, 2H), 4.70 (td, 2H), 7.44 (t, 1H), 7.52 (d, 1H), 7.71 (d, 1H), |
| | 7.85 (s, 1H), 7.91 (d, 1H) |
| 1-272 | 1H-NMR (CDCl3) δ: 3.75 (d, 1H), 4.13 (d, 1H), 4.28 (t, 2H), 4.02 (s, 2H), 7.43 (t, 1H), 7.51 (d, 1H), 7.73 (d, 1H), 7.88 (s, 1H), |
| | 7.95 (d, 1H) |
| 1-295 | 1H-NMR (CDCl3) δ: 3.82 (1H, d), 4.21 (1H, d), 7.62-7.39 (4H, m), 8.25-7.93 (4H, m). |
| 3-1 | 1H-NMR (CDCl3) δ: 1.64-1.80 (m, 1H), 2.10 (bs, 2H), 2.47-2.61 (m, 1H), 2.75-2.90 (m, 1H), 2.90-3.05 (m, 1H), 3.69 (d, 1H), 4.08 |
| | (d, 1H), 4.38 (t, 1H), 7.35-7.44 (m, 2H), 7.46-7.57 (m, 4H) |
| 3-3 | 1H-NMR (CDCl3) δ: 1.74-1.91 (m, 1H), 2.06 (s, 3H), 2.57-2.70 (m, 1H), 2.82-3.07 (m, 2H), 3.69 (d, 1H), 4.07 (d, 1H), 5.52 (q, 1H), |
| | 5.60-5.67 (d, 1H), 7.34 (d, 1H), 7.40-7.61 (m, 5H) |
| 3-4 | 1H-NMR (CDCl3) δ: 1.21 (t, 3H), 1.71-1.88 (m, 1H), 2.27 (q, 2H), 2.55-2.70 (m, 1H), 2.80-3.06 (m, 2H), 3.69 (d, 1H), 4.08 (d, 1H), |
| | 5.52 (q, 1H), 5.63 (d, 1H), 7.28-7.61 (m, 6H) |
| 3-6 | 1H-NMR (CDCl3) δ: 1.77-1.93 (m, 1H), 2.57-2.73 (m, 1H), 2.76-3.04 (m, 1H), 3.13 (q, 1H), 3.68 (d, 1H), 4.07 (d, 1H), 5.52 (q, 1H), |
| | 6.00 (m, 1H), 7.30 (d, 1H), 7.39-7.61 (m, 5H) |
| 3-7 | 1H-NMR (CDCl3) δ: 0.72-0.85 (m, 2H), 0.98-1.10 (m, 2H), 1.30-1.42 (m, 1H), 1.75-1.92 (m, 1H), 2.55-2.71 (m, 1H), 2.80-3.07 (m, 2H), |
| | 3.69 (d, 1H), 4.08 (d, 1H), 5.53 (q, 1H), 5.83 (d, 1H), 7.32-7.63 (m, 6H) |
| 3-8 | 1H-NMR (CDCl3) δ: 1.67-1.85 (m, 1H), 2.54-2.67 (m, 1H), 2.73-3.01 (m, 5H), 3.69 (d, 1H), 4.07 (d, 1H), 4.37-4.49 (m, 1H), 4.55-4.65 |
| | (m, 1H), 5.24-5.38 (m, 1H), 7.29-7.57 (m, 6H) |
| 3-9 | 1H-NMR (CDCl3) δ: 1.16 (t, 3H), 1.69-1.84 (m, 1H), 2.55-2.67 (m, 1H), 2.75-3.02 (m, 2H), 3.24 (quintet, 1H), 3.68 (d, 1H), 4.07 |
| | (d, 1H), 4.37 (bs, 1H), 4.49-4.57 (m, 1H), 5.33 (q, 1H), 7.35 (d, 1H), 7.40-7.57 (m, 5H) |
| 3-23 | 1H-NMR (CDCl3) δ: 1.81-1.98 (m, 1H), 2.67-2.81 (m, 1H), 2.82-2.98 (m, 2H), 3.00 (s, 3H), 3.69 (d, 1H), 5.85 (bs, 1H), 5.91 (bs, 1H), |
| | 6.10 (bs, 1H), 7.39-7.60 (m, 6H) |
| 3-32 | 1H-NMR (CDCl3) δ: 1.81-1.97 (m, 1H), 2.63-2.77 (m, 1H), 2.81-3.07 (q, 2H), 3.54-3.87 (m, 4H), 4.06 (d, 1H), 4.70 (t, 2H), 5.86 |
| | (bs, 1H), 6.62 (bs, 1H), 6.83 (bs, 1H), 7.39-7.58 (m, 6H) |
| 3-33 | 1H-NMR (CDCl3) δ: 2.42-2.66 (m, 2H), 2.95-3.09 (m, 1H), 3.31-3.43 (m, 1H), 3.68 (d, 1H), 4.07 (m, 1H), 5.89 (t, 1H), 7.15 (s, 1H), |
| | 7.42 (s, 1H), 7.45 (d, 1H), 7.50 (s, 2H), 7.61 (d, 1H), 7.70-7.78 (m, 2H), 7.80-7.90 (m, 2H) |
| 3-34 | 1H-NMR (CDCl3) δ: 1.90-2.03 (m, 1H), 2.48-2.60 (m, 1H), 2.48-2.60 (m, 1H), 2.77-2.90 (m, 1H), 3.00-3.13 (m, 1H), 3.70 (d, 1H), 4.09 |
| | (d, 1H), 5.27 (t, 1H), 7.40-7.59 (m, 6H) |
| 3-39 | 1H-NMR (CDCl3) δ: 1.74-1.89 (m, 1H), 2.07 (s, 3H), 2.55-2.70 (m, 1H), 2.79-3.07 (m, 2H), 3.68 (d, 1H), 4.08 (d, 1H), 5.51 (q, 1H), |
| | 5.61-5.74 (m, 1H), 7.30-7.60 (m, 3H), 7.64 (s, 2H) |
| 3-40 | 1H-NMR (CDCl3) δ: 1.21 (t, 3H), 1.72-1.88 (m, 1H), 2.27 (q, 2H), 2.56-2.72 (m, 1H), 2.80-3.06 (m, 2H), 3.68 (d, 1H), 4.08 (d, 1H), |
| | 5.47-5.68 (m, 2H), 7.28-7.59 (m, 3H), 7.64 (s, 2H) |
| 3-42 | 1H-NMR (CDCl3) δ: 1.75-1.91 (m, 1H), 2.54-2.69 (m, 1H), 2.80-3.04 (m, 2H), 3.11 (q, 2H), 3.68 (d, 1H), 4.07 (d, 1H), 5.49 (q, 1H), |
| | 6.15-6.27 (m, 1H), 7.22-7.58 (m, 3H), 7.64 (s, 2H) |
| 3-43 | 1H-NMR (CDCl3) δ: 0.72-0.82 (m, 1H), 0.86-0.95 (m, 1H), 0.98-1.09 (m, 2H), 1.32-1.43 (m, 1H), 1.76-1.91 (m, 1H), 2.54-2.68 (m, 1H), |
| | 2.78-3.06 (m, 2H), 3.69 (s, 1H), 4.08 (s, 1H), 5.51 (q, 1H), 5.97 (d, 1H), 7.28-7.58 (m, 3H), 7.64 (s, 2H) |
| 3-44 | 1H-NMR (CDCl3) δ: 1.62-1.78 (m, 1H), 2.40-2.61 (m, 1H), 2.65-2.95 (m, 5H), 3.69 (d, 1H), 4.06 (d, 1H), 4.70-5.01 (m, 2H), 5.15-5.32 |
| | (m, 1H), 7.22-7.49M, 3H), 7.65 (s, 2H) |
| 3-45 | 1H-NMR (CDCl3) δ: 1.10 (t, 3H), 1.56-1.76 (m, 1H), 2.43-2.57 (m, 1H), 2.64-2.92 (m, 2H), 3.18 (q, 2H), 3.70 (d, 1H), 4.06 (d, 1H), |
| | 4.81-4.92 (m, 1H), 5.02-5.31 (m, 2H), 7.19-7.45 (m, 3H), 7.65 (s, 2H) |
| 3-48 | 1H-NMR (CDCl3) δ: 0.53-0.62 (m, 2H), 0.68-0.78 (m, 2H), 1.73-1.90 (m, 1H), 2.38-2.50 (m, 1H), 2.59-2.74 (m, 1H), 2.79-3.06 (m, 2H), |
| | 3.69 (d, 1H), 4.08 (d, 1H), 4.87 (bs, 1H), 5.20 (d, 1H), 5.42 (q, 1H), 7.32-7.58 (m, 3H), 7.65 (s, 2H) |
| 3-55 | 1H-NMR (CDCl3) δ: 1.57-1.76 (m, 1H), 2.42-2.58 (m, 1H), 2.63-2.93 (m, 2H), 3.69 (d, 1H), 3.75-3.90 (m, 2H), 4.06 (d, 1H), 5.11-5.36 |
| | (m, 3H), 7.15-7.47 (m, 3H), 7.64 (s, 2H) |
| 3-57 | 1H-NMR (CDCl3) δ: 1.68-1.86 (m, 1H), 2.18-2.25 (m, 1H), 2.51-2.67 (m, 1H), 2.73-3.01 (m, 2H), 3.68 (d, 1H), 3.93-4.07 (m, 3H), |
| | 4.68-4.86 (m, 2H), 5.31 (q, 1H), 7.29-7.54 (m, 3H), 7.64 (s, 2H) |
| 3-61 | 1H-NMR (CDCl3) δ: 1.73-1.86 (m, 1H), 2.57-2.73 (m, 1H), 2.80-3.01 (m, 8H), 3.67 (d, 1H), 4.08 (d, 1H), 4.52 (d, 1H), 5.43 (q, 1H), |
| | 7.34-7.59 (m, 3H), 7.64 (s, 2H) |
| 3-70 | 1H-NMR (CDCl3) δ: 2.07 (s, 3H), 2.08-2.19 (m, 1H), 2.46-2.60 (m, 1H), 2.84-2.96 (m, 1H), 3.06-3.19 (m, 1H), 3.68 (d, 1H), 6.18 |
| | (dd, 1H), 7.46-7.52 (m, 2H), 7.60 (d, 1H), 7.64 (s, 2H) |
| 3-73 | 1H-NMR (CDCl3) δ: 1.20 (t, 3H), 1.73-1.89 (m, 1H), 2.27 (q, 2H), 2.55-2.69 (m, 1H), 2.79-3.06 (m, 2H), 3.70 (d, 1H), 4.14 (d, 1H), |
| | 5.51 (q, 1H), 5.65-5.78 (m, 1H), 7.28-7.60 (m, 3H), 7.84 (s, 1H), 7.95 (s, 1H) |
| 3-74 | 1H-NMR (CDCl3) δ: 1.77-1.93 (m, 1H), 2.57-2.75 (m, 1H), 2.82-3.23 (m, 4H), 3.70 (d, 1H), 4.13 (d, 1H), 5.53 (q, 1H), 5.89-6.09 |
| | (m, 1H), 7.26-7.64 (m, 3H), 7.84 (s, 1H), 7.95 (s, 1H) |
| 3-81 | 1H-NMR (CDCl3) δ: 1.21 (t, 3H), 1.72-1.89 (m, 1H), 2.27 (q, 2H), 2.55-2.70 (m, 1H), 2.81-3.07 (m, 2H), 3.69 (d, 1H), 4.07 (d, 1H), |
| | 5.53 (q, 1H), 5.61-5.72 (m, 1H), 7.27-7.60 (m, 3H), 7.67-7.80 (m, 3H) |
| 3-82 | 1H-NMR (CDCl3) δ: 1.93-2.10 (m, 1H), 2.64-3.11 (m, 3H), 3.69 (d, 1H), 4.08 (d, 1H), 4.87 (d, 1H), 5.12 (q, 1H), 6.28 (t, 1H), |
| | 7.45-7.74 (m, 5H) |
| 3-88 | 1H-NMR (CDCl3) δ: 1.74-1.91 (m, 1H), 2.05 (s, 3H), 2.57-2.70 (m, 1H), 2.81-3.07 (m, 2H), 3.74 (d, 1H), 4.19 (d, 1H), 5.51 (q, 1H), |
| | 5.66-5.74 (m, 1H), 7.30-7.62 (m, 3H), 7.96 (s, 1H), 8.09 (s, 2H) |
| 3-89 | 1H-NMR (CDCl3) δ: 1.20 (t, 3H), 1.72-1.89 (m, 1H), 2.27 (q, 1H), 2.55-2.70 (m, 1H), 2.81-3.06 (m, 2H), 3.75 (d, 1H), 4.20 (d, 1H), |
| | 5.52 (q, 1H), 5.72 (d, 1H), 7.28-7.62 (m, 3H), 7.96 (s, 1H), 8.09 (s, 2H) |
| 3-90 | 1H-NMR (CDCl3) δ: 1.78-1.96 (m, 1H), 2.59-2.75 (m, 1H), 2.83-3.20 (m, 4H), 3.75 (d, 1H), 4.19 (d, 1H), 5.55 (q, 1H), 5.82-5.96 |
| | (m, 1H), 7.17-7.64 (m, 3H), 7.96 (s, 1H), 8.08 (s, 2H) |
| 3-91 | 1H-NMR (CDCl3) δ: 0.73-0.83 (m, 1H), 0.87-0.97 (m, 1H), 1.00-1.10 (m, 2H), 1.30-1.40 (m, 1H), 1.76-1.91 (m, 1H), 2.58-2.70 (m, 1H), |
| | 2.81-3.07 (m, 2H), 3.74 (d, 1H), 4.19 (d, 1H), 5.54 (q, 1H), 5.73-5.84 (m, 1H), 7.32-7.61 (m, 3H), 7.96 (s, 1H), 8.09 (s, 2H) |
| 3-92 | 1H-NMR (CDCl3) δ: 1.58-1.91 (m, 1H), 2.35-2.97 (m, 6H), 3.75 (s, 1H), 4.19 (d, 1H), 4.92-5.30 (m, 3H), 7.18-7.49 (m, 3H), 7.96 |
| | (s, 1H), 8.08 (s, 2H) |
| 3-93 | 1H-NMR (CDCl3) δ: 1.16 (t, 3H), 1.70-1.86 (m, 1H), 2.55-2.71 (m, 1H), 2.77-3.02 (m, 2H), 3.24 (q, 2H), 3.74 (d, 1H), 4.19 (d, 1H), |
| | 4.27-4.36 (m, 1H), 4.43-4.52 (m, 1H), 5.35 (q, 1H), 7.34-7.70 (m, 3H), 7.96 (s, 1H), 8.09 (s, 2H) |
| 3-95 | 1H-NMR (CDCl3) δ: 1.81-2.00 (m, 1H), 2.66-3.13 (m, 6H), 3.74 (d, 1H), 4.19 (d, 1H), 5.64-6.11 (m, 3H), 7.40-7.65 (m, 3H), 7.96 |
| | (s, 1H), 8.09 (s, 2H) |
| 3-97 | 1H-NMR (CDCl3) δ: 1.20 (t, 3H), 1.72-1.88 (m, 1H), 2.27 (q, 2H), 2.54-2.70 (m, 1H), 2.80-3.07 (m, 2H), 3.72 (d, 1H), 4.14 (d, 1H), |
| | 5.52 (q, 1H), 5.63-5.75 (m, 1H), 7.28-7.59 (m, 3H), 7.68 (s, 1H), 7.76 (s, 1H), 7.82 (s, 1H) |
| 3-98 | 1H-NMR (CDCl3) δ: 1.77-1.93 (m, 1H), 2.57-2.73 (m, 1H), 2.82-3.21 (m, 4H), 3.71 (d, 1H), 4.13 (d, 1H), 5.52 (q, 1H), 5.97-6.10 |
| | (m, 1H), 7.27-7.61 (m, 6H), 7.68 (s, 1H), 7.76 (s, 1H), 7.82 (s, 1H) |
| 3-104 | 1H-NMR (CDCl3) δ: 1.85-1.95 (1H, m), 2.51-2.55 (1H, m), 2.82-3.46 (5H, m), 3.77 (1H, d), 4.36-4.52 (2H, m), 4.80-4.85 (1H, m), |
| | 7.24-7.28 (2H, m), 7.36-7.37 (1H, m), 7.45 (1H, d, J = 7.9 Hz), 7.69-7.72 (2H, m) |
| 3-106 | 1H-NMR (CDCl3) δ: 1.82-1.86 (1H, m), 2.04 (3H, s), 2.61-2.65 (1H, m), 2.89-2.97 (2H, m), 3.44 (1H, d), 3.79 (1H, d), 4.42 (1H, d), |
| | 4.87 (1H, d), 5.50-5.53 (1H, m), 5.81-5.84 (1H, m), 7.29-7.34 (4H, m), 7.65-7.76 (2H, m) |
| 3-112 | 1H-NMR (CDCl3) δ: 1.11 (3H, t), 1.65-1.82 (1H, m), 2.51-2.56 (1H, m), 2.77-2.89 (2H, m), 3.17-3.22 (2H, m), 3.42 (1H, d), 3.77 |
| | (1H, d), 4.40 (1H, d), 4.81-4.86 (2H, m), 4.98 (1H, d), 5.25-5.28 (1H, m), 7.27-7.37 (4H, m), 7.62-7.68 (2H, m) |
| 3-130 | 1H-NMR (CDCl3) δ: 1.49 (9H, s), 1.78-1.88 (1H, m), 2.59-2.62 (1H, m), 2.80-3.02 (2H, m), 3.44 (1H, d), 3.79 (1H, d), 4.41 (1H, d), |
| | 4.83-4.89 (2H, m), 5.19-5.22 (1H, m), 7.26-7.29 (2H, m), 7.36-7.39 (2H, m), 7.66-7.73 (2H, m) |
| 3-137 | 1H-NMR (CDCl3) δ: 1.89-2.06 (2H, m), 2.50-2.63 (1H, m), 2.81-2.91 (1H, m), 3.09-3.13 (1H, m), 3.44-3.47 (1H, m), 3.77-3.83 (1H, m), |
| | 4.43 (1H, d), 4.84-4.90 (1H, m), 5.28 (1H, br s), 7.23-7.29 (3H, m), 7.37-7.38 (1H, m), 7.49 (1H, d), 7.72-7.75 (2H, m) |
| 3-138 | 1H-NMR (CDCl3) δ: 2.04-2.19 (4H, m), 2.53-2.57 (1H, m), 2.94-2.96 (1H, m), 3.11-3.16 (1H, m), 3.45 (1H, d), 3.80 (1H, d), 4.44 |
| | (1H, d), 4.85-4.91 (1H, m), 6.20-6.22 (1H, m), 7.26-7.27 (2H, m), 7.37-7.38 (1H, m), 7.48 (1H, d), 7.69-7.72 (1H, m), 7.79-7.80 |
| 3-140 | 1H-NMR (CDCl3) δ: 1.76-1.86 (1H, m), 2.38-2.58 (1H, m), 2.83-3.07 (2H, m), 3.43 (1H, d), 3.79 (1H, d), 4.41-4.43 (3H, m), 4.85 |
| | (1H, d), 7.40-7.44 (3H, m), 7.70-7.75 (2H, m) |
| 3-142 | 1H-NMR (CDCl3) δ: 1.79-1.89 (1H, m), 2.05 (3H, s), 2.58-2.69 (1H, m), 2.90-3.01 (2H, m), 3.43 (1H, d), 3.79 (1H, d), 4.41 (1H, d), |
| | 4.86 (1H, dd), 5.51-5.53 (1H, m), 5.75-5.78 (1H, m), 7.36-7.39 (3H, m), 7.65-7.76 (2H, m) |
| 3-143 | 1H-NMR (CDCl3) δ: 1.21 (3H, t), 1.79-1.89 (1H, m), 2.28 (2H, q), 2.63-2.68 (1H, m), 2.93-2.98 (2H, m), 3.44 (1H, d), 3.79 (1H, d), |
| | 4.42 (1H, d), 4.85-4.88 (1H, m), 5.54-5.56 (1H, m), 5.74-5.77 (1H, m), 7.35-7.39 (3H, m), 7.65-7.75 (2H, m) |
| 3-145 | 1H-NMR (CDCl3) δ: 1.81-1.91 (1H, m), 2.61-2.64 (1H, m), 2.87-3.23 (4H, m), 3.46 (1H, d), 3.80 (1H, d), 4.43 (1H, d), 4.87 (1H, d), |
| | 5.48-5.51 (1H, m), 6.35-6.37 (2H, m), 7.31 (1H, d), 7.40 (2H, s), 7.63-7.73 (2H, m) |
| 3-166 | 1H-NMR (CDCl3) δ: 1.50 (9H, s), 1.79-1.89 (1H, m), 2.61-2.63 (1H, m), 2.82-3.03 (2H, m), 3.43 (1H, d), 3.79 (1H, d), 4.42 (1H, d), |
| | 4.80-4.86 (2H, m), 5.21-5.23 (1H, m), 7.38-7.41 (3H, m), 7.69-7.72 (2H, m) |
| 3-188 | 1H-NMR (CDCl3) δ: 1.83-1.88 (1H, m), 2.06 (3H, s), 2.63-2.67 (1H, m), 2.86-3.07 (2H, m), 3.49-3.53 (1H, m), 3.91 (1H, d), 4.50 |
| | (1H, d), 4.98-5.01 (1H, m), 5.52-5.55 (1H, m), 5.67-5.70 (1H, m), 7.38 (1H, d), 7.69-7.91 (5H, m) |
| 3-191 | 1H-NMR (CDCl3) δ: 0.81-0.91 (4H, m), 1.23-1.41 (1H, m), 1.83-1.89 (1H, m), 2.61-2.69 (1H, m), 2.92-3.00 (2H, m), 3.51 (1H, d), 3.91 |
| | (1H, d), 4.50 (1H, d), 4.99 (1H, d), 5.52-5.62 (1H, m), 5.85-5.88 (1H, m), 7.39 (1H, d), 7.69-7.91 (5H, m) |
| 3-211 | 1H-NMR (CDCl3) δ: 1.47-2.11 (m, 6H), 2.68-2.90 (m, 2H), 3.68 (d, 1H), 3.98 (t, 1H), 4.07 (d, 1H), 7.37 (s, 1H), 7.42 (t, 1H), 7.48 |
| | (s, 2H), 7.51 (s, 2H) |
| 3-213 | 1H-NMR (CDCl3) δ: 1.71-1.92 (m, 3H), 1.99-2.13 (m, 4H), 2.70-2.89 (m, 2H), 3.67 (d, 1H), 4.06 (d, 1H), 5.16-5.28 (m, 1H), 5.68 |
| | (d, 1H), 7.31-7.54 (m, 6H) |
| 3-216 | 1H-NMR (CDCl3) δ: 1.76-1.94 (m, 3H), 2.06-2.19 (m, 1H), 3.67 (d, 1H), 4.06 (d, 1H), 4.13 (s, 2H), 5.16-5.27 (m, 1H), 6.74 (d, 1H), |
| | 7.29-7.53 (m, 6H) |
| 3-217 | 1H-NMR (CDCl3) δ: 1.73-1.93 (m, 3H), 2.01-2.18 (m, 1H), 2.69-2.89 (m, 2H), 3.11 (q, 2H), 3.67 (d, 1H), 4.05 (d, 1H), 5.16-5.28 |
| | (m, 1H), 5.99 (d, 1H), 7.22-7.56 (m, 6H) |
| 3-218 | 1H-NMR (CDCl3) δ: 0.71-0.83 (m, 2H), 0.97-1.09 (m, 2H), 1.28-1.39 (m, 1H), 1.71-1.92 (m, 3H), 1.99-2.16 (m, 1H), 2.70-2.90 (m, 2H), |
| | 3.67 (d, 1H), 4.06 (d, 1H), 5.16-5.28 (m, 1H), 5.84 (d, 1H), 7.31-7.55 (m, 6H) |
| 3-219 | 1H-NMR (CDCl3) δ: 1.85-1.99 (m, 3H), 2.10-2.27 (m, 1H), 2.76-2.91 (m, 2H), 3.68 (d, 1H), 4.07 (d, 1H), 5.37-5.50 (m, 1H), 6.32 |
| | (d, 1H), 7.36-7.61 (m, 9H), 7.80 (d, 2H) |
| 3-220 | 1H-NMR (CDCl3) δ: 1.71-1.91 (m, 3H), 2.01-2.13 (m, 1H), 2.70-2.88 (m, 5H), 3.67 (d, 1H), 4.06 (d, 1H), 4.24-4.36 (m, 1H), 4.49 |
| | (d, 1H), 4.95-5.08 (m, 1H), 7.32-7.56 (m, 6H) |
| 3-221 | 1H-NMR (CDCl3) δ: 1.16 (t, 3H), 1.69-1.91 (m, 3H), 2.03-2.14 (m, 1H), 2.71-2.83 (m, 2H), 3.23 (q, 2H), 3.66 (d, 1H), 4.06 (d, 1H), |
| | 4.21-4.29 (m, 1H), 4.43 (d, 1H), 4.97-5.07 (m, 1H), 7.32-7.54 (m, 6H) |
| 3-230 | 1H-NMR (CDCl3) δ: 1.69-1.91 (m, 3H), 1.99-2.14 (m, 1H), 2.71-2.84 (m, 2H), 3.52-3.71 (m, 5H), 4.06 (d, 1H), 4.60 (d, 1H), 4.71-4.80 |
| | (m, 1H), 4.94-5.5 (m, 1H), 7.32-7.46 (m, 4H), 7.51 (s, 2H) |
| 3-235 | 1H-NMR (CDCl3) δ: 0.60-0.85 (m, 4H), 1.81-1.96 (m, 3H), 2.18-2.31 (m, 1H), 2.38-2.49 (m, 1H), 2.75-2.93 (m, 2H), 3.68 (d, 1H), 4.07 |
| | (d, 1H), 5.75-5.86 (m, 1H), 6.33-6.44 (m, 2H), 7.39-7.59 (m, 6H) |
| 3-236 | 1H-NMR (CDCl3) δ: 1.79-1.93 (m, 3H), 2.12-2.27 (m, 1H), 2.74-2.86 (m, 1H), 2.96 (s, 3H), 3.67 (d, 1H), 4.06 (d, 1H), 5.67 (bs, 1H), |
| | 5.81 (bs, 1H), 6.12 (bs, 1H), 7.35-7.54 (m, 6H) |
| 3-238 | 1H-NMR (CDCl3) δ: 1.71-1.92 (m, 3H), 1.98-2.14 (m, 1H), 2.72-2.84 (m, 2H), 2.92 (s, 6H), 3.68 (d, 1H), 4.06 (d, 1H), 4.56 (d, 1H), |
| | 5.03-5.15 (m, 1H), 7.33-7.56 (m, 6H) |
| 3-239 | 1H-NMR (CDCl3) δ: 1.75-1.91 (m, 3H), 2.03-2.14 (m, 1H), 2.71-2.85 (m, 2H), 3.66 (d, 1H), 3.72 (s, 3H), 4.06 (d, 1H), 4.90 (s, 2H), |
| | 7.34-7.54 (m, 6H) |
| 3-241 | 1H-NMR (CDCl3) δ: 1.79-1.98 (m, 3H), 2.06-2.25 (m, 1H), 2.70-2.90 (m, 2H), 3.08 (s, 3H), 3.67 (d, 1H), 4.06 (d, 1H), 4.50 (d, 1H), |
| | 4.62-4.75 (m, 1H), 7.36-7.61 (m, 6H) |
| 3-251 | 1H-NMR (CDCl3) δ: 1.48-2.11 (m, 6H), 2.66-2.90 (m, 2H), 3.67 (d, 1H), 3.98 (t, 1H), 4.08 (d, 1H), 7.37 (s, 1H), 7.48 (s, 2H), 7.65 |
| | (s, 2H) |
| 3-253 | |
| 3-254 | 1H-NMR (CDCl3) δ: 1.20 (t, 3H), 1.68-1.93 (m, 3H), 2.00-2.13 (m, 1H), 2.26 (q, 2H), 2.70-2.91 (m, 2H), 3.66 (d, 1H), 4.06 (d, 1H), |
| | 5.15-5.29 (m, 1H), 5.58-5.70 (m, 1H), 7.28-7.50 (m, 3H), 7.64 (s, 2H) |
| 3-256 | 1H-NMR (CDCl3) δ: 1.80-1.87 (3H, m), 2.05-2.13 (1H, m), 2.80-2.82 (2H, m), 3.11 (2H, q), 3.66 (1H, d), 4.06 (2H, dd), 5.21-5.26 |
| | (1H, m), 5.95 (1H, d), 7.30 (1H, d), 7.39-7.51 (2H, m), 7.64 (2H, s) |
| 3-257 | 1H-NMR (CDCl3) δ: 0.70-0.83 (m, 2H), 0.94-1.12 (m, 2H), 1.27-1.40 (m, 1H), 1.70-1.93 (m, 3H), 1.99-2.14 (m, 1H), 2.67-2.92 (m, 2H), |
| | 3.66 (d, 1H), 4.06 (d, 1H), 5.15-5.29 (m, 1H), 5.84 (d, 1H), 7.31-7.54 (m, 3H), 7.64 (s, 2H) |
| 3-258 | 1H-NMR (CDCl3) δ: 1.63-1.87 (m, 3H), 1.95-2.10 (m, 1H), 2.64-2.86 (m, 5H), 3.66 (d, 1H), 4.05 (d, 1H), 4.48-4.58 (m, 1H), 4.66-4.77 |
| | (m, 1H), 4.92-5.04 (m, 1H), 7.27-7.48 (m, 3H), 7.64 (s, 2H) |
| 3-259 | 1H-NMR (CDCl3) δ: 1.13 (t, 3H), 1.65-1.88 (m, 3H), 1.95-2.12 (m, 1H), 2.63-2.85 (m, 2H), 3.11-3.28 (m, 2H), 3.66 (d, 1H), 4.06 |
| | (d, 1H), 4.43-4.53 (m, 1H), 4.63-4.74 (m, 1H), 4.91-5.04 (m, 1H), 7.26-7.46 (m, 3H), 7.64 (s, 2H) |
| 3-261 | 1H-NMR (CDCl3) δ: 1.09-1.19 (m, 6H), 1.65-1.88 (m, 3H), 2.00-2.13 (m, 1H), 2.66-2.81 (m, 2H), 3.65 (d, 1H), 3.78-3.93 (m, 1H), 4.05 |
| | (d, 1H), 4.24 (d, 1H), 4.53 (d, 1H), 4.91-5.05 (m, 1H), 7.28-7.46 (m, 3H), 7.64 (s, 2H) |
| 3-270 | 1H-NMR (CDCl3) δ: 1.65-1.88 (m, 3H), 1.96-2.10 (m, 1H), 2.65-2.83 (m, 2H), 3.66 (d, 1H), 3.73-3.84 (m, 2H), 4.05 (d, 1H), 4.57-4.70 |
| | (m, 1H), 4.73-4.83 (m, 1H), 4.91-5.03 (m, 1H), 5.05-5.24 (m, 2H), 5.75-5.93 (m, 1H), 7.27-7.46 (m, 3H), 7.64 (s, 2H) |
| 3-284 | 1H-NMR (CDCl3) δ: 1.76-2.19 (m, 7H), 2.65-2.95 (m, 2H), 3.67 (d, 1H), 4.07 (d, 1H), 5.95-6.03 (m, 1H), 7.30-7.52 (m, 3H), 7.64 |
| | (s, 2H) |
| 3-302 | 1H-NMR (CDCl3) δ: 1.67-1.93 (m, 3H), 1.99-2.14 (m, 4H), 2.72-2.90 (m, 2H), 3.72 (d, 1H), 4.17 (d, 1H), 5.16-5.28 (m, 1H), 5.64 |
| | (d, 1H), 7.31-7.53 (m, 3H), 7.96 (s, 1H), 8.08 (s, 2H) |
| 3-303 | 1H-NMR (CDCl3) δ: 1.20 (t, 3H), 1.68-1.92 (m, 3H), 1.99-2.14 (m, 1H), 2.26 (q, 2H), 2.69-2.90 (m, 2H), 3.72 (d, 1H), 4.18 (d, 1H), |
| | 5.15-5.28 (m, 1H), 5.56-5.69 (m, 1H), 7.29-7.53 (m, 3H), 7.96 (s, 1H), 8.08 (s, 2H) |
| 3-304 | 1H-NMR (CDCl3) δ: 1.72-1.92 (m, 3H), 1.98-2.17 (m, 1H), 2.70-2.89 (m, 2H), 3.11 (q, 2H), 3.72 (d, 1H), 4.17 (d, 1H), 5.16-5.29 |
| | (m, 1H), 5.92-6.06 (m, 1H), 7.25-7.52 (m, 3H), 7.96 (s, 1H), 8.08 (s, 2H) |
| 3-305 | 1H-NMR (CDCl3) δ: 0.70-0.84 (m, 2H), 0.96-1.11 (m, 2H), 1.28-1.39 (m, 1H), 1.72-1.93 (m, 3H), 2.01-2.15 (m, 1H), 2.68-2.93 (m, 2H), |
| | 3.72 (d, 1H), 4.17 (d, 1H), 5.17-5.29 (m, 1H), 5.82 (d, 1H), 7.32-7.54 (m, 3H), 7.96 (s, 1H), 8.09 (s, 2H) |
| 3-306 | 1H-NMR (CDCl3) δ: 1.62-1.90 (m, 3H), 1.98-2.11 (m, 1H), 2.68-2.87 (m, 5H), 3.72 (d, 1H), 4.19 (d, 1H), 4.37-4.52 (m, 1H), 4.63 |
| | (d, 1H), 4.91-5.08 (m, 1H), 7.28-7.51 (m, 3H), 7.96 (s, 1H), 8.08 (s, 2H) |
| 3-307 | 1H-NMR (CDCl3) δ: 1.14 (t, 3H), 1.54-1.87 (m, 3H), 1.98-2.13 (m, 1H), 2.68-2.86 (m, 2H), 3.14-3.30 (m, 2H), 3.72 (d, 1H), 4.18 |
| | (d, 1H), 4.27-4.65 (m, 2H), 4.94-5.09 (m, 1H), 7.29-7.52 (m, 3H), 7.96 (s, 1H), 8.08 (s, 2H) |
| 3-318 | 1H-NMR (CDCl3) δ: 1.91-2.10 (4H, m), 2.80-2.86 (2H, m), 3.40 (1H, d), 3.72 (1H, d), 4.32-4.36 (2H, m), 4.78 (1H, d), 7.27-7.45 |
| | (4H, m), 7.58-7.61 (2H, m). |
| 3-320 | 1H-NMR (CDCl3) δ: 1.66-1.70 (2H, m), 1.78-1.90 (2H, m), 2.08 (3H, s), 2.82-2.84 (2H, m), 3.42 (1H, d), 3.77 (1H, d), 4.42 (1H, d), |
| | 4.86 (1H, d), 5.21-5.24 (1H, m), 5.72 (1H, d), 7.26-7.38 (4H, m), 7.59-7.63 (2H, m). |
| 3-322 | 1H-NMR (CDCl3) δ: 1.90-1.92 (3H, m), 2.12-2.17 (1H, m), 2.87-2.89 (2H, m), 3.43 (1H, d), 3.77 (1H, d), 4.43 (1H, d), 4.88 (1H, d), |
| | 5.26-5.29 (1H, m), 6.46 (1H, d), 7.28-7.36 (4H, m), 7.65-7.67 (2H, m). |
| 3-323 | 1H-NMR (CDCl3) δ: 1.84-1.87 (2H, m), 2.07-2.14 (2H, m), 2.84-2.86 (2H, m), 3.12 (2H, q), 3.43 (1H, d), 3.77 (1H, d), 4.42 (1H, d), |
| | 4.87 (1H, d), 5.25-5.28 (1H, m), 5.93 (1H, d), 7.29-7.36 (4H, m), 7.62-7.65 (2H, m). |
| 3-326 | 1H-NMR (CDCl3) δ: 1.13 (3H, t), 1.71-2.06 (3H, m), 2.79 (2H, s), 3.21 (2H, q), 3.41 (1H, d), 3.76 (1H, d), 4.40 (1H, d), 4.47-4.49 |
| | (1H, m), 4.71 (1H, d), 4.85 (1H, d), 5.00 (1H, d), 7.31-7.38 (4H, m), 7.54-7.58 (2H, m). |
| 3-344 | 1H-NMR (CDCl3) δ: 1.23-1.28 (2H, m), 1.46 (9H, s), 1.82-1.84 (2H, m), 2.79-2.81 (2H, m), 3.42 (1H, d), 3.77 (1H, d), 4.41 (1H, d), |
| | 4.81-4.87 (3H, m), 7.31-7.39 (4H, m), 7.57-7.64 (2H, m). |
| 3-355 | 1H-NMR (CDCl3) δ: 1.79-1.87 (4H, m), 2.05 (3H, s), 2.82-2.84 (2H, m), 3.41 (1H, d), 3.77 (1H, d), 4.41 (1H, d), 4.85 (1H, d), |
| | 5.22-5.24 (1H, m), 5.74 (1H, d), 7.36-7.39 (3H, m), 7.59-7.63 (2H, m). |
| 3-358 | 1H-NMR (CDCl3) δ: 1.83-1.87 (4H, m), 2.83-2.84 (2H, m), 3.12 (2H, q), 3.41 (1H, d), 3.77 (1H, d), 4.41 (1H, d), 4.86 (1H, d), |
| | 5.25-5.27 (1H, m), 5.96 (1H, d), 7.33-7.38 (3H, m), 7.61-7.64 (2H, m). |
| 3-361 | 1H-NMR (CDCl3) δ: 1.11 (3H, t), 1.72-1.80 (4H, m), 2.74-2.76 (2H, m), 3.16-3.21 (2H, m), 3.39 (1H, d), 3.75 (1H, d), 4.39 (1H, d), |
| | 4.62-4.63 (1H, m), 4.82-4.85 (2H, m), 4.96-4.99 (1H, m), 7.39-7.41 (3H, m), 7.51-7.60 (2H, m). |
| 3-922 | 1H-NMR (CDCl3) δ: 1.49 (9H, s), 1.79-1.84 (4H, m), 2.80-2.82 (2H, m), 3.42 (1H, d), 3.78 (1H, d), 4.41 (1H, d), 4.84-4.87 (3H, m), |
| | 7.41-7.45 (3H, m), 7.57-7.64 (2H, m). |
| 3-547 | 1H-NMR (CDCl3) δ: 1.04-1.29 (3H, m), 2.38 (2H, q), 3.56 (1H, d), 3.94 (1H, d), 4.49 (1H, d), 4.94 (1H, d), 7.48-7.58 (4H, m), |
| | 7.74-7.76 (1H, m), 7.89-7.94 (2H, m), 8.10-8.14 (2H, m). |
| 3-549 | 1H-NMR (CDCl3) δ: 0.92-0.95 (2H, m), 1.12-1.17 (2H, m), 1.67-1.70 (1H, m), 3.55 (1H, d), 3.93 (1H, d), 4.48 (1H, d), 4.94 (1H, d), |
| | 7.46-7.51 (4H, m), 7.71-7.74 (1H, m), 7.94-7.99 (2H, m), 8.09-8.12 (2H, m). |
| 3-551 | 1H-NMR (CDCl3) δ: 1.07-1.13 (3H, m), 3.26-3.30 (2H, m), 3.55 (1H, d), 3.92 (1H, d), 4.47 (1H, d), 4.83-4.86 (1H, m), 4.93 (1H, d), |
| | 6.78 (1H, s), 7.44 (2H, s), 7.51 (1H, t), 7.66 (1H, d), 7.77 (1H, d), 8.06 (2H, s), 8.17 (1H, s). |
| 3-635 | 1H-NMR (CDCl3) δ: 3.14 (t, 2H), 3.67 (d, 1H), 3.85 (s, 3H), 3.99-4.12 (m, 3H), 7.33-7.44 (m, 2H), 7.51 (s, 2H), 7.58 (s, 1H), 7.97 |
| | (m, 1H) |
| 3-636 | 1H-NMR (CDCl3) δ: 1.57 (s, 9H), 3.11 (t, 2H), 3.67 (d, 1H), 3.94-4.06 (m, 3H), 7.30-7.59 (m, 6H) |
| 3-637 | 1H-NMR (acetone-d6) δ: 2.78 (3H, d,), 3.19 (2H, t), 4.00 (2H, t), 4.14 (1H, d), 4.31 (1H, d), 6.05 (1H, br s), 7.46 (1H, dd), |
| | 7.56-7.56 (1H, m), 7.61 (1H, t), 7.66 (2H, t), 8.01 (1H, d). |
| 3-638 | 1H-NMR (CDCl3) δ: 1.22 (t, 3H), 3.20 (t, 2H), 3.39 (quintet, 2H), 3.68 (d, 1H), 3.93 (t, 2H), 4.07 (d, 1H), 4.57-4.66 (m, 1H), 7.31 |
| | (d, 1H), 7.41 (s, 1H), 7.51 (s, 2H), 7.58 (s, 1H), 7.98 (d, 1H) |
| 3-640 | 1H-NMR (CDCl3) δ: 1.23 (d, 6H), 3.20 (t, 2H), 3.68 (d, 1H), 3.92 (t, 2H), 4.01-4.15 (m, 2H), 4.40 (d, 1H), 7.30 (d, 1H), 7.41 |
| | (s, 1H), 7.51 (s, 2H), 7.57 (s, 1H), 7.97 (d, 1H) |
| 3-641 | 1H-NMR (CDCl3) δ: 0.54-0.60 (2H, m), 0.81-0.83 (2H, m), 2.71-2.75 (1H, m), 3.20 (2H, t), 3.67 (1H, d), 3.88 (2H), 4.07 (1H, d), |
| | 4.85 (1H, s), 7.31 (1H, dd), 7.41 (1H, t), 7.51 (2H, d), 7.58 (1H, d), 8.00 (1H, d). |
| 3-642 | 1H-NMR (CDCl3) δ: 0.22-0.27 (2H, m), 0.51-0.57 (2H, m), 1.02-1.03 (1H, m), 3.19-3.23 (4H, m), 3.67 (1H, d), 3.97 (2H, t,), 4.07 |
| | (1H, d), 4.71 (1H, t), 7.31 (1H, dd), 7.41 (1H, t), 7.51-7.51 (2H, m), 7.58-7.58 (1H, m), 7.97 (1H, d). |
| 3-643 | 1H-NMR (CDCl3) δ: 3.26 (3H, t), 3.68 (1H, d), 4.01-4.07 (6H, m), 4.89 (1H, t), 7.34 (1H, dd), 7.41-7.42 (1H, m), 7.51-7.51 (2H, m), |
| | 7.61 (1H, d), 7.96 (1H, d). |
| 3-644 | 1H-NMR (CDCl3) δ: 3.23 (2H, t), 3.68 (1H, d), 4.06-4.11 (3H, m), 4.64 (2H, t), 6.21 (1H, d), 7.22 (1H, dd), 7.32-7.33 (2H, m), 7.41 |
| | (1H, t), 7.52-7.52 (2H, m), 7.59-7.59 (1H, m), 7.69 (1H, td), 8.01 (1H, d), 8.55 (1H, dd). |
| 3-645 | 1H-NMR (CDCl3) δ: 3.22 (t, 2H), 3.67 (d, 1H), 3.91-4.02 (m, 4H), 4.07 (d, 1H), 4.64-4.67 (m, 1H), 5.12-5.32 (m, 2H), 5.85-6.01 |
| | (m, 1H), 7.31 (d, 1H), 7.41 (s, 1H), 7.51 (s, 2H), 7.59 (s, 1H), 7.98 (d, 1H) |
| 3-646 | 1H-NMR (CDCl3) δ: 2.27 (1H, t), 3.22 (2H, t), 3.68 (1H, d), 3.97 (2H, t), 4.04-4.16 (3H, m), 4.82 (1H, t), 7.32 (1H, dd), 7.41 |
| | (1H, t), 7.51-7.51 (2H, m), 7.59-7.59 (1H, m), 7.98 (1H, d). |
| 3-647 | 1H-NMR (acetone-d6) δ: 3.34 (2H, t), 4.18 (1H, d), 4.34-4.36 (3H, m), 7.12-7.14 (1H), 7.31-7.36 (1H), 7.44-7.47 (1H), 7.53-7.56 (1H), |
| | 7.62-7.65 (4H), 8.03-8.05 (1H, d), 8.13-8.15 (1H). |
| 3-648 | 1H-NMR (acetone-d6) δ: 3.28 (2H, t), 4.17 (1H, d), 4.24-4.37 (3H, m), 6.99-7.84 (14H, m), 8.05 (1H, d). |
| 3-649 | 1H-NMR (acetone-d6) δ: 3.28 (2H, t), 4.17 (1H, d), 4.27 (2H, t), 4.34 (0H, d), 7.12-7.32 (5H, m), 7.56-7.61 (4H, m), 8.05 (1H, d), |
| | 8.11 (1H, s) |
| 3-650 | 1H-NMR (CDCl3) δ: 3.22 (3H, t), 3.67 (1H, d), 3.96 (2H, t), 4.07 (1H, d), 4.50 (2H, d), 4.92 (1H, t), 7.26-7.34 (5H, m), 7.41 |
| | (1H, t), 7.51-7.52 (2H, m), 7.59-7.60 (1H, m), 7.99 (1H, d). |
| 3-651 | 1H-NMR (CDCl3) δ: 2.95 (s, 6H), 3.06 (t, 2H), 3.66 (d, 1H), 3.95 (t, 2H), 4.06 (d, 1H), 6.94 (d, 1H), 7.36 (d, 1H), 7.41 (s, 1H), |
| | 7.51 (s, 2H), 7.54 (s, 1H) |
| 3-652 | 1H-NMR (CDCl3) δ: 3.13 (t, 2H), 3.24 (s, 3H), 3.67 (d, 1H), 4.06 (d, 1H), 4.32 (t, 2H), 6.05-6.17 (m, 1H), 7.34-7.44 (m, 2H), 7.51 |
| | (s, 2H), 7.61 (s, 1H), 8.08 (d, 1H) |
| 3-656 | 1H-NMR (CDCl3) δ: 1.15 (3H, t), 1.89-1.98 (2H, m), 2.76 (2H, t), 3.28-3.38 (2H, m), 3.64-3.75 (3H, m), 4.06 (1H, d), 5.02 (1H, t), |
| | 7.44-7.48 (7H, m). |
| 3-659 | 1H-NMR (CDCl3) δ: 0.50-0.52 (2H), 0.75-0.77 (2H), 1.92-1.95 (2H), 2.70-2.77 (3H, m), 3.63-3.74 (3H, m), 4.06 (1H, dz), 5.22 (1H, s), |
| | 7.35-7.51 (6H, m). |
| 3-660 | 1H-NMR (CDCl3) δ: 0.18-0.20 (2H, m), 0.49-0.52 (2H, m), 0.95-1.00 (1H, m), 1.90-1.99 (2H, m), 2.77 (2H, t), 3.16 (2H, dd), 3.65-3.76 |
| | (3H, m), 4.07 (1H, d,), 5.15 (1H, t), 7.43-7.50 (6H, m). |
| 3-661A | 1H-NMR (CDCl3) δ: 1.92-2.00 (2H, m), 2.78 (2H, t), 3.65-4.15 (6H, m), 5.38 (1H, t), 7.41-7.46 (6H, m). |
| 3-663 | 1H-NMR (CDCl3) δ: 1.91-1.99 (2H), 2.77 (2H, t), 3.67 (1H, d), 3.75 (2H, t), 3.91-3.93 (2H), 4.06 (1H, d), 5.09-5.20 (3H, m), |
| | 5.82-5.93 (1H, m), 7.43-7.50 (6H). |
| 3-664 | 1H-NMR (CDCl3) δ: 1.95-1.97 (2H), 2.24 (1H, t), 2.77 (2H, t), 3.67 (1H, d), 3.75 (2H, t), 4.06-4.09 (3H), 5.21 (1H), 7.43-7.50 (6H). |
| 3-777 | 1H-NMR (CDCl3) δ: 3.81 (d, 1H), 4.20 (d, 1H), 7.40-7.49 (m, 2H), 7.54 (s, 2H), 7.96 (s, 1H), 8.01-8.22 (m, 3H) |
| 3-193 | 1H-NMR (CDCl3) δ: 1.13 (3H, t), 1.72-1.77 (1H, m), 2.55-2.62 (1H, m), 2.81-2.90 (2H, m), 3.19-3.24 (2H, m), 3.49 (1H, d), 3.90 |
| | (1H, d), 4.48 (1H, d), 4.64-4.66 (1H, m), 4.83 (1H, d), 4.97 (1H, d), 5.29-5.31 (1H, m), 7.36 (1H, d), 7.63-7.72 (2H, m), |
| | 7.82 (2H, s), 7.91 (1H, s) |
| 3-778 | 1H-NMR (CDCl3) δ: 2.21 (3H, s), 3.79 (1H, d, J = 17.2 Hz), 4.16 (2H, d, J = 17.2 Hz), 4.69 (2H, s), 7.41-7.42 (1H, m), |
| | 7.52-7.55 (3H, m), 7.93-7.94 (1H, m), 8.03-8.06 (1H, m) |
| 3-779 | 1H-NMR (CDCl3) δ: 3.76 (d, 1H), 4.15 (d, 1H), 6.78 (d, 1H), 7.41-7.50 (m, 2H), 7.53 (s, 2H), 7.78-7.94 (m, 3H) |
| 3-781 | 1H-NMR (CDCl3) δ: 2.10 (3H, d), 3.67-3.84 (1H, m), 4.08-4.21 (1H, m), 4.56 (1H, d), 6.99-7.07 (2H, m), 7.29-7.54 (4H, m), 8.35 |
| | (1H, s). |
| 3-782 | 1H-NMR (CDCl3) δ: 3.67 (1H, d), 4.05 (1H, d), 7.10-7.13 (1H, m), 7.28-7.31 (1H, m), 7.43-7.44 (1H, m), 7.50-7.51 (3H, m). |
| 3-784 | 1H-NMR (CDCl3) δ: 3.22 (s, 6H), 3.75 (d, 1H), 3.90 (s, 2H), 4.14 (d, 1H), 7.43 (s, 1H), 7.52 (s, 2H), 7.56-7.63 (m, 2H), 7.83 |
| | (s, 1H), 7.87 (d, 1H) |
| 3-785 | 1H-NMR (CDCl3) δ: 3.70-4.18 (m), 5.07 (m), 6.50 (m), 7.27-7.92 (m), 10.11 (s) |
| 3-786 | 1H-NMR (CDCl3) δ: 1.25 (3H, t), 3.42-3.49 (2H, m), 3.74 (1H, d), 4.13 (1H, d), 4.93 (2H, s), 7.44-7.44 (1H, m), 7.51 (2H, s), |
| | 7.66 (1H, d), 7.76 (1H, s), 7.82-7.83 (2H, m), 9.71 (1H, br s) |
| 3-793 | 1H-NMR (CDCl3) δ: 1.13 (t, 3H), 1.60-1.81 (m, 1H), 2.47-2.62 (m, 1H), 2.70-2.97 (m, 2H), 3.14-3.28 (m, 2H), 3.73 (d, 1H), 4.12 |
| | (d, 1H), 4.53-4.64 (m, 1H), 4.71-7.82 (m, 1H), 5.27 (q, 1H), 7.28-7.93 (m, 7H) |
| 3-828 | 1H-NMR (CDCl3) δ: 1.64-1.84 (4H), 1.89-1.97 (2H, m), 2.31-2.38 (2H, m), 2.76 (2H, t), 3.65-3.73 (3H), 4.07 (1H, d), 4.35-4.38 |
| | (1H, m), 5.17 (1H, d), 7.40-7.52 (6H) |
| 3-829 | 1H-NMR (CDCl3) δ: 1.71-1.92 (m, 3H), 1.98-2.14 (m, 1H), 2.72-2.84 (m, 2H), 2.92 (s, 6H), 3.68 (d, 1H), 4.06 (d, 1H), 4.56 (d, 1H), |
| | 5.03-5.15 (m, 1H), 7.33-7.56 (m, 6H) |
| 3-830 | 1H-NMR (CDCl3) δ: 2.02-2.20 (m, 1H), 2.45-2.62 (m, 1H), 2.83-2.98 (m, 1H), 3.06-3.21 (m, 1H), 3.74 (d, 1H), 4.20 (d, 1H), 6.14-6.23 |
| | (m, 1H), 7.42-7.64 (m, 3H), 7.96 (s, 1H), 8.08 (s, 2H) |
| 3-831 | 1H-NMR (CDCl3) δ: 1.58-1.81 (m, 3H), 2.48-2.61 (m, 1H), 2.75-3.06 (m, 2H), 3.75 (d, 1H), 4.20 (d, 1H), 4.38 (t, 1H), 7.34-7.61 |
| | (m, 3H), 7.96 (s, 1H), 8.09 (s, 2H) |
| 3-832 | 1H-NMR (CDCl3) δ: 1.74-1.88 (m, 1H), 2.56-2.73 (m, 1H), 2.78-3.07 (m, 8H), 3.74 (d, 1H), 4.20 (d, 1H), 4.56 (d, 1H), 5.42 (q, 1H), |
| | 7.34-7.61 (m, 3H), 7.96 (s, 1H), 8.09 (s, 2H) |
| 3-833 | 1H-NMR (CDCl3) δ: 1.89-2.06 (m, 1H), 2.66-2.82 (m, 1H), 2.87-3.13 (m, 2H), 3.76 (d, 1H), 4.21 (d, 1H), 5.73 (q, 1H), 6.44 (d, 1H), |
| | 7.26-7.76 (m, 7H), 7.96 (s, 1H), 8.09 (s, 2H) |
| 3-834 | 1H-NMR (CDCl3) δ: 1.93-1.99 (1H, m), 2.66-2.76 (1H, m), 2.94-3.05 (2H, m), 3.76 (1H, dd), 4.20 (1H, dd), 5.67-5.70 (1H, m), 6.44-6.47 |
| | (1H, m), 7.28-7.39 (2H, m), 7.45-7.67 (4H, m), 7.76 (1H, s), 7.97 (1H, s), 8.09 (2H, s) |
| 3-835 | 1H-NMR (CDCl3) δ: 1.72-2.05 (m, 3H), 2.09-2.40 (m, 4H), 2.54-2.70 (m, 1H), 2.79-3.12 (m, 3H), 3.75 (d, 1H), 4.20 (d, 1H), 5.44-5.63 |
| | (m, 2H), 7.21-7.63 (m, 3H), 7.96 (s, 1H), 8.09 (s, 2H) |
| 3-836 | 1H-NMR (CDCl3) δ: 1.69-1.85 (m, 1H), 2.51-2.68 (m, 1H), 2.75-3.02 (m, 2H), 3.74 (d, 1H), 3.81-3.97 (m, 2H), 4.19 (d, 1H), 4.78-4.94 |
| | (m, 1H), 5.23-5.37 (m, 1H), 7.28-7.56 (m, 3H), 7.96 (s, 1H), 8.08 (s, 2H) |
| 3-837 | 1H-NMR (CDCl3) δ: 1.71-1.88 (m, 1H), 2.53-2.68 (m, 1H), 2.75-3.03 (m, 2H), 3.74 (d, 1H), 4.19 (d, 1H), 4.48 (bs, 1H), 4.76-4.90 |
| | (m, 1H), 5.28 (q, 1H), 7.32-7.59 (m, 3H), 7.96 (s, 1H), 8.08 (s, 2H) |
| 3-838 | 1H-NMR (CDCl3) δ: 0.50-0.63 (m, 2H), 0.67-0.79 (m, 2H), 1.74-1.91 (m, 1H), 2.38-2.51 (m, 1H), 2.59-2.74 (m, 1H), 2.78-3.08 (m, 2H), |
| | 3.76 (d, 1H), 4.21 (d, 1H), 4.91 (s, 1H), 5.20 (d, 1H), 5.42 (q, 1H), 7.33-7.65 (m, 3H), 7.96 (s, 1H), 8.09 (s, 2H) |
| 3-839 | 1H-NMR (CDCl3) δ: 1.69-1.86 (m, 1H), 2.20-2.35 (m, 2H), 2.56-2.70 (m, 1H), 2.77-3.04 (m, 2H), 3.74 (d, 1H), 3.98 (t, 4H), 4.14-4.25 |
| | (m, 2H), 5.39 (q, 1H), 7.34-7.60 (m, 3H), 7.96 (s, 1H), 8.09 (s, 2H) |
| 3-840 | 1H-NMR (CDCl3) δ: 1.50 (9H, s), 1.81-1.87 (1H, m), 2.60-2.63 (1H, m), 2.88-2.97 (2H, m), 3.51 (1H, d), 3.92 (1H, d), 4.49 (1H, d), |
| | 4.85-4.88 (1H, m), 4.99 (1H, d), 5.20-5.23 (1H, m), 7.40 (1H, d), 7.70-7.75 (2H, m), 7.84 (2H, s), 7.91 (1H, s) |
| 3-841 | 1H-NMR (CDCl3) δ: 1.67-1.86 (m, 1H), 2.50-2.66 (m, 1H), 2.73-3.01 (m, 2H), 3.68 (d, 1H), 4.08 (d, 1H), 4.54 (bs, 2H), 4.89-5.00 |
| | (m, 1H), 5.18-5.34 (m, 1H), 7.29-7.56 (m, 3H), 7.64 (s, 2H) |
| 3-842 | 1H-NMR (CDCl3) δ: 1.93-2.10 (m, 1H), 2.64-3.11 (m, 3H), 3.69 (d, 1H), 4.08 (d, 1H), 4.87 (d, 1H), 5.12 (q, 1H), 6.28 (t, 1H), |
| | 7.45-7.74 (m, 5H) |
| 3-843 | 1H-NMR (CDCl3) δ: 1.90-2.06 (m, 1H), 2.59-3.12 (m, 3H), 3.68 (d, 1H), 3.82-3.97 (m, 2H), 4.08 (d, 1H), 4.87 (d, 1H), 5.04 (q, 1H), |
| | 7.41-7.68 (m, 5H) |
| 3-844 | 1H-NMR (CDCl3) δ: 1.72-1.99 (m, 5H), 2.57-2.73 (m, 1H), 2.77-3.04 (m, 2H), 3.26-3.45 (m, 4H), 3.69 (d, 1H), 4.08 (d, 1H), 4.38 |
| | (d, 1H), 5.45 (q, 1H), 7.35-7.58 (m, 3H), 7.65 (s, 2H) |
| 3-845 | 1H-NMR (CDCl3) δ: 1.77-1.96 (m, 1H), 2.29 (s, 3H), 2.54-2.70 (m, 1H), 2.80-3.09 (m, 2H), 3.49 (s, 2H), 3.67 (d, 1H), 4.08 (d, 1H), |
| | 5.50 (q, 1H), 7.27-7.59 (m, 3H), 7.65 (s, 2H) |
| 3-846 | 1H-NMR (CDCl3) δ: 1.80-1.98 (m, 1H), 2.57-2.73 (m, 1H), 2.83-3.12 (m, 2H), 3.45 (s, 2H), 3.69 (d, 1H), 4.08 (d, 1H), 5.49 (q, 1H), |
| | 6.35 (d, 1H), 7.28-7.61 (m, 3H), 7.65 (s, 2H) |
| 3-847 | 1H-NMR (CDCl3) δ: 1.79-1.94 (m, 1H), 2.57-2.74 (m, 1H), 2.82-3.08 (m, 2H), 3.68 (d, 1H), 4.08 (d, 1H), 5.61 (q, 1H), 5.67-5.84 |
| | (m, 2H), 6.12 (dd 1H), 6.36 (dd, 1H), 7.30-7.67 (m, 5H) |
| 3-848 | 1H-NMR (CDCl3) δ: 1.86-2.03 (m, 1H), 2.61-2.78 (m, 1H), 2.86-3.13 (m, 2H), 3.69 (d, 1H), 4.08 (d, 1H), 5.69 (q, 1H), 6.46-6.59 |
| | (m, 2H), 7.14-7.20 (m, 1H), 7.39-7.71 (m, 6H) |
| 3-849 | 1H-NMR (CDCl3) δ: 1.62-1.80 (m, 1H), 2.40-2.58 (m, 1H), 2.62-2.92 (m, 2H), 3.69 (d, 1H), 3.99-4.18 (m, 3H), 5.09-5.41 (m, 3H), |
| | 7.15-7.46 (m, 3H), 7.64 (s, 2H) |
| 3-850 | 1H-NMR (CDCl3) δ: 1.76-1.93 (m, 1H), 2.28 (s, 6H), 2.55-2.70 (m, 1H), 2.80-3.09 (m, 5H), 3.68 (d, 1H), 4.08 (d, 1H), 5.54 (q, 1H), |
| | 7.27-7.60 (m, 3H), 7.64 (s, 2H) |
| 3-851 | 1H-NMR (CDCl3) δ: 0.13-0.22 (m, 2H), 0.43-0.54 (m, 2H), 0.86-1.03 (m, 1H), 1.69-1.84 (m, 1H), 2.52-2.67 (m, 1H), 2.73-2.99 (m, 2H), |
| | 3.06 (t, 2H), 3.68 (d, 1H), 4.07 (d, 1H), 4.59-4.76 (m, 2H), 5.32 (q, 1H), 7.31-7.54 (m, 3H), 7.65 (s, 2H) |
| 3-852 | 1H-NMR (CDCl3) δ: 0.98 (t, 3H), 1.63-1.88 (m, 3H), 2.22 (t, 2H), 2.55-2.70 (m, 1H), 2.80-3.06 (m, 2H), 3.68 (d, 1H), 4.08 (d, 1H), |
| | 5.53 (q, 1H), 5.68 (d, 1H), 7.28-7.58 (m, 3H), 7.64 (s, 2H) |
| 3-853 | 1H-NMR (CDCl3) δ: 1.79-1.94 (m, 1H), 2.14 (s, 3H), 2.58-2.74 (m, 1H), 2.83-3.11 (m, 2H), 3.27 (s, 2H), 3.69 (d, 1H), 4.09 (d, 1H), |
| | 5.52 (q, 1H), 7.08 (d, 1H), 7.28-7.72 (m, 5H) |
| 3-854 | 1H-NMR (CDCl3) δ: 1.81-1.98 (m, 1H), 2.57-2.72 (m, 1H), 2.83-3.07 (m, 2H), 3.11 (s, 3H), 3.68 (d, 1H), 4.07 (d, 1H), 5.51 (q, 1H), |
| | 6.57 (d, 1H), 7.32-7.60 (m, 3H), 7.64 (s, 2H) |
| 3-855 | 1H-NMR (CDCl3) δ: 1.63-1.94 (m, 2H), 2.09-2.36 (m, 2H), 2.56-2.72 (m, 1H), 2.81-3.10 (m, 2H), 3.68 (d, 1H), 4.07 (d, 1H), 5.55 |
| | (q, 1H), 5.93 (d, 1H), 7.28-7.69 (m, 5H) |
| 3-856 | 1H-NMR (CDCl3) δ: 0.87-1.06 (m, 1H), 1.23-1.46 (m, 1H), 1.69-1.90 (m, 1H), 2.54-2.72 (m, 1H), 2.76-3.06 (m, 3H), 3.68 (d, 1H), |
| | 4.07 (d, 1H), 4.39-4.69 (m, 1H), 4.77 (s, 1H), 5.04 (d, 1H), 5.38 (q, 1H), 7.30-7.59 (m, 3H), 7.65 (s, 2H) |
| 3-857 | 1H-NMR (CDCl3) δ: 0.29 (s, 9H), 2.33-2.50 (m, 1H), 2.77-2.92 (m, 1H), 2.98-3.31 (m, 2H), 3.71 (d, 1H), 4.09 (d, 1H), 6.30 (t, 1H), |
| | 7.20 (d, 1H), 7.27 (s, 1H), 7.40-7.57 (m, 4H), 7.71 (d, 1H) |
| 3-858 | 1H-NMR (CDCl3) δ: 2.35-2.51 (m, 1H), 2.80-2.95 (m, 1H), 3.01-3.31 (m, 2H), 3.71 (d, 1H), 4.09 (d, 1H), 6.22-6.32 (m, 1H), 7.14-7.77 |
| | (m, 7H) |
| 3-859 | 1H-NMR (CDCl3) δ: 1.64-1.82 (m, 1H), 2.48-2.65 (m, 1H), 2.70-2.96 (m, 2H), 3.68 (d, 1H), 4.06 (d, 1H), 5.12-5.25 (m, 1H), 5.29-5.44 |
| | (m, 1H), 6.60 (m, 1H), 6.97-7.58 (m, 11H) |
| 3-860 | 1H-NMR (CDCl3) δ: 1.76-1.94 (m, 1H), 2.57-2.73 (m, 1H), 2.79-3.06 (m, 2H), 3.69 (d, 1H), 4.07 (d, 1H), 5.09-5.20 (m, 1H), 5.42 |
| | (q, 1H), 6.80 (s, 1H), 6.91-7.03 (m, 1H), 7.18-7.61 (m, 8H), 8.07-8.18 (m, 1H) |
| 3-861 | 1H-NMR (CDCl3) δ: 1.77-1.93 (m, 1H), 2.59-2.74 (m, 1H), 2.80-3.06 (m, 2H), 3.69 (d, 1H), 4.07 (d, 1H), 4.89-5.00 (m, 1H), 5.36-5.48 |
| | (m, 1H), 6.52 (s, 1H), 7.13-7.64 (m, 9H), 7.95-8.03 (m, 1H) |
| 3-862 | 1H-NMR (CDCl3) δ: 1.79-1.97 (m, 1H), 2.58-2.74 (m, 1H), 2.81-3.10 (m, 2H), 3.61-3.78 (m, 4H), 4.08 (d, 1H), 5.43 (q, 1H), 5.89 |
| | (d, 1H), 7.16 (s, 1H), 7.33-7.63 (m, 6H) |
| 3-863 | 1H-NMR (CDCl3) δ: 1.21 (t, 3H), 1.76-1.92 (m, 1H), 2.57-2.71 (m, 1H), 2.79-3.05 (m, 2H), 3.67 (d, 1H), 3.85 (q, 2H), 4.05 (d, 1H), |
| | 5.40 (q, 1H), 5.87 (d, 1H), 7.15 (s, 1H), 7.30-7.62 (m, 6H) |
| 3-864 | 1H-NMR (CDCl3) δ: 1.78-1.94 (m, 1H), 2.57-2.73 (m, 1H), 2.80-3.08 (m, 2H), 3.70 (d, 1H), 4.08 (d, 1H), 4.29 (d, 2H), 5.24-5.51 |
| | (m, 3H), 5.80-6.03 (m, 2H), 7.21 (s, 1H), 7.32-7.65 (m, 6H) |
| 3-865 | 1H-NMR (CDCl3) δ: 1.75-1.93 (m, 1H), 2.55-2.72 (m, 1H), 2.79-3.06 (m, 2H), 3.15 (s, 3H), 3.56-3.77 (m, 4H), 4.07 (d, 1H), 5.38 |
| | (q, 1H), 5.96 (d, 1H), 7.31-7.62 (m, 6H) |
| 3-866 | 1H-NMR (CDCl3) δ: 1.45 (d, 3H), 1.71-1.90 (m, 1H), 2.28 (s, 3H), 2.54-2.70 (m, 1H), 2.80-3.08 (m, 2H), 3.45 (q, 1H), 3.68 (d, 1H), |
| | 4.07 (d, 1H), 5.40-5.55 (m, 1H), 6.40-6.54 (m, 1H), 7.21-7.60 (m, 6H) |
| 3-867 | 1H-NMR (CDCl3) δ: 1.65-1.97 (m, 5H), 2.19-2.43 (m, 1H), 2.54-2.71 (m, 1H), 2.80-3.14 (m, 4H), 3.69 (d, 1H), 4.07 (d, 1H), 5.51 |
| | (q, 1H), 7.20-7.63 (m, 6H) |
| 3-868 | 1H-NMR (CDCl3) δ: 0.17-0.27 (m, 2H), 0.56-0.66 (m, 2H), 0.91-1.06 (m, 1H), 1.74-1.90 (m, 1H), 2.18-2.30 (m, 2H), 2.60-2.73 (m, 1H), |
| | 2.82-3.08 (m, 2H), 3.69 (d, 1H), 4.08 (d, 1H), 5.56 (q, 1H), 6.05 (d, 1H), 7.29-7.61 (m, 6H) |
| 3-869 | 1H-NMR (CDCl3) δ: 0.93 (t, 3H), 1.30-1.45 (m, 2H), 1.58-1.87 (m, 3H), 2.23 (t, 2H), 2.55-2.70 (m, 1H), 2.80-3.06 (m, 2H), 3.69 |
| | (d, 1H), 4.08 (d, 1H), 5.52 (q, 1H), 5.68 (d, 1H), 7.28-7.61 (m, 6H) |
| 3-870 | 1H-NMR (CDCl3) δ: 0.89 (t, 3H), 1.41-1.55 (m, 2H), 1.60-1.77 (m, 1H), 2.42-2.57 (m, 1H), 2.65-2.92 (m, 2H), 3.05-3.18 (m, 2H), 3.69 |
| | (d, 1H), 4.06 (d, 1H), 4.83-5.29 (m, 3H), 7.21-7.47 (m, 4H), 7.51 (s, 2H) |
| 3-871 | 1H-NMR (CDCl3) δ: 2.28-2.54 (m, 2H), 2.65-2.78 (m, 4H), 2.87-3.05 (m, 1H), 3.23-3.40 (m, 1H), 3.67 (d, 1H), 4.07 (d, 1H), 5.68-5.78 |
| | (m, 1H), 7.04-7.13 (m, 1H), 7.37-7.60 (m, 5H) |
| 3-872 | 1H-NMR (CDCl3) δ: 1.79-1.94 (m, 1H), 2.14 (s, 3H), 2.59-2.74 (m, 1H), 2.84-3.10 (m, 2H), 3.27 (s, 2H), 3.70 (d, 1H), 4.08 (d, 1H), |
| | 5.53 (q, 1H), 7.02-7.14 (m, 1H), 7.28-7.64 (m, 6H) |
| 3-873 | 1H-NMR (CDCl3) δ: 1.84-2.01 (m, 1H), 2.56-2.77 (m, 4H), 2.82-3.10 (m, 2H), 3.29 (d, 1H), 3.62-3.81 (m, 2H), 4.07 (d, 1H), 5.53 |
| | (q, 1H), 7.12-7.65 (m, 7H) |
| 3-874 | 1H-NMR (CDCl3) δ: 1.81-1.97 (m, 1H), 2.55-2.70 (m, 1H), 2.81-3.12 (m, 5H), 3.69 (d, 1H), 3.90 (s, 2H), 4.07 (d, 1H), 5.49 (q, 1H), |
| | 6.67 (d, 1H), 7.29-7.61 (m, 6H) |
| 3-875 | 1H-NMR (CDCl3) δ: 1.22-1.32 (m, 3H), 1.77-1.95 (m, 1H), 2.50-2.72 (m, 3H), 2.82-3.11 (m, 2H), 3.22-3.38 (m, 2H), 3.70 (d, 1H), 4.08 |
| | (d, 1H), 5.51 (q, 1H), 7.05-7.16 (m, 1H), 7.26-7.64 (m, 6H) |
| 3-876 | 1H-NMR (CDCl3) δ: 1.71-1.90 (m, 1H), 2.55-2.71 (m, 1H), 2.80-3.14 (m, 4H), 3.69 (d, 1H), 4.08 (d, 1H), 5.18-5.31 (m, 2H), 5.52 |
| | (q, 1H), 5.77 (d, 1H), 5.86-6.04 (m, 1H), 7.24-7.63 (m, 6H) |
| 3-877 | 1H-NMR (CDCl3) δ: 1.68-1.84 (m, 1H), 2.18-2.25 (m, 1H), 2.51-2.66 (m, 1H), 2.72-2.99 (m, 2H), 3.69 (d, 1H), 3.94-4.12 (m, 3H), |
| | 4.69-4.87 (m, 2H), 5.31 (q, 1H), 7.28-7.57 (m, 6H) |
| 3-878 | 1H-NMR (CDCl3) δ: 0.98 (t, 3H), 1.65-1.89 (m, 3H), 2.21 (t, 2H), 2.55-2.70 (m, 1H), 2.81-3.06 (m, 2H), 3.69 (d, 1H), 4.08 (d, 1H), |
| | 5.52 (q, 1H), 5.70 (d, 1H), 7.26-7.62 (m, 6H) |
| 3-879 | 1H-NMR (CDCl3) δ: 2.08-2.23 (m, 1H), 2.42-2.58 (m, 1H), 2.83-3.17 (m, 2H), 3.69 (d, 1H), 4.08 (d, 1H), 4.81-4.93 (m, 1H), 7.37-7.66 |
| | (m, 6H) |
| 3-880 | 1H-NMR (CDCl3) δ: 1.76-1.95 (m, 1H), 2.57-2.78 (m, 1H), 2.82-3.24 (m, 4H), 3.69 (d, 1H), 4.10 (d, 1H), 5.53 (q, 1H), 5.98 (d, 1H), |
| | 7.26-7.62 (m, 3H), 7.68 (s, 2H) |
| 3-881 | 1H-NMR (CDCl3) δ: 1.75-1.94 (m, 1H), 2.56-2.72 (m, 1H), 2.81-3.24 (m, 4H), 3.71 (d, 1H), 4.12 (d, 1H), 5.52 (q, 1H), 5.96-6.14 |
| | (m, 1H), 7.23-7.90 (m, 6H) |
| 3-882 | 1H-NMR (CDCl3) δ: 1.75-1.92 (m, 1H), 2.54-2.73 (m, 1H), 2.79-3.21 (m, 4H), 3.71 (d, 1H), 4.07 (d, 1H), 5.51 (q, 1H), 6.01-6.16 |
| | (m, 1H), 7.21-7.65 (m, 7H) |
| 3-883 | 1H-NMR (CDCl3) δ: 1.77-1.93 (m, 1H), 2.56-2.73 (m, 1H), 2.82-3.21 (m, 4H), 3.68 (d, 1H), 4.06 (d, 1H), 5.53 (q, 1H), 5.96 (d, 1H), |
| | 6.80-6.94 (m, 1H), 7.08-7.63 (m, 5H) |
| 3-884 | 1H-NMR (CDCl3) δ: 1.75-1.92 (m, 1H), 2.55-2.72 (m, 1H), 2.81-3.23 (m, 4H), 3.73 (d, 1H), 4.12 (d, 1H), 5.52 (q, 1H), 5.94-6.13 |
| | (m, 1H), 7.20-7.85 (m, 7H) |
| 3-885 | 1H-NMR (CDCl3) δ: 1.77-1.94 (m, 1H), 2.55-2.71 (m, 1H), 2.84-3.21 (m, 4H), 3.77 (d, 1H), 4.17 (d, 1H), 5.52 (q, 1H), 6.01-6.18 |
| | (m, 1H), 7.24-7.76 (m, 4H), 7.92-8.07 (m, 1H), 8.26-8.56 (m, 2H) |
| 3-886 | 1H-NMR (CDCl3) δ: 1.76-1.93 (m, 1H), 2.53-2.69 (m, 1H), 2.81-3.23 (m, 4H), 3.71 (d, 1H), 4.13 (d, 1H), 5.49 (q, 1H), 6.24-6.37 |
| | (m, 1H), 7.24-7.97 (m, 7H) |
| 3-887 | 1H-NMR (CDCl3) δ: 1.75-1.93 (m, 1H), 2.53-2.73 (m, 1H), 2.81-3.21 (m, 4H), 3.74 (d, 1H), 4.13 (d, 1H), 5.52 (q, 1H), 5.89-6.09 |
| | (m, 1H), 7.21-7.91 (m, 7H) |
| 3-888 | 1H-NMR (CDCl3) δ: 1.77-1.94 (m, 1H), 2.60-2.74 (m, 1H), 2.83-3.21 (m, 4H), 3.71 (d, 1H), 4.11 (d, 1H), 5.55 (q, 1H), 5.82-5.95 |
| | (m, 1H), 7.27-7.82 (m, 6H) |
| 3-889 | 1H-NMR (CDCl3) δ: 1.75-1.93 (m, 1H), 2.52 (s, 3H), 2.57-2.73 (m, 1H), 2.81-3.21 (m, 4H), 3.73 (d, 1H), 4.06 (d, 1H), 5.53 (q, 1H), |
| | 5.89-6.07 (m, 1H), 7.21-7.64 (m, 7H) |
| 3-890 | 1H-NMR (CDCl3) δ: 1.74-1.92 (m, 1H), 2.55-2.70 (m, 1H), 2.79-3.21 (m, 4H), 3.69 (d, 1H), 4.07 (d, 1H), 5.50 (q, 1H), 6.02-6.17 |
| | (m, 1H), 7.23-7.77 (m, 6H) |
| 3-891 | 1H-NMR (CDCl3) δ: 1.76-1.92 (m, 1H), 2.55-2.72 (m, 1H), 2.81-3.21 (m, 4H), 3.66 (d, 1H), 4.06 (d, 1H), 5.52 (q, 1H), 5.96-6.11 |
| | (m, 1H), 7.20-7.61 (m, 5H) |
| 3-892 | 1H-NMR (CDCl3) δ: 1.77-1.93 (m, 1H), 2.54-2.75 (m, 1H), 2.83-3.22 (m, 4H), 3.77 (d, 1H), 4.23 (d, 1H), 5.55 (q, 1H), 5.81-5.95 |
| | (m, 1H), 7.29-8.72 (m, 6H) |
| 3-893 | 1H-NMR (CDCl3) δ: 1.75-1.93 (m, 1H), 2.56-2.71 (m, 1H), 2.81-3.21 (m, 4H), 3.71 (d, 1H), 4.09 (d, 1H), 5.51 (q, 1H), 5.96-6.11 |
| | (m, 1H), 7.21-7.62 (m, 7H) |
| 3-894 | 1H-NMR (CDCl3) δ: 1.75-1.92 (m, 1H), 2.53-2.68 (m, 1H), 2.78-3.20 (m, 4H), 3.73 (d, 1H), 3.84 (s, 3H), 4.04 (d, 1H), 5.48 (q, 1H), |
| | 6.11-6.24 (m, 1H), 6.88-7.60 (m, 6H) |
| 3-895 | 1H-NMR (CDCl3) δ: 1.20 (t, 3H), 1.73-1.90 (m, 1H), 2.27 (q, 2H), 2.54-2.70 (m, 1H), 2.78-3.06 (m, 2H), 3.74 (d, 1H), 4.16 (d, 1H), |
| | 5.52 (q, 1H), 5.63-5.77 (m, 1H), 7.28-7.59 (m, 3H), 7.82 (d, 2H), 8.30 (d, 2H) |
| 3-896 | 1H-NMR (CDCl3) δ: 1.20 (t, 3H), 1.73-1.89 (m, 1H), 2.27 (q, 2H), 2.53 (s, 3H), 2.56-2.70 (m, 1H), 2.81-3.08 (m, 2H), 3.75 (d, 1H), |
| | 4.14 (d, 1H), 5.52 (q, 1H), 5.63-5.75 (m, 1H), 7.29-7.70 (m, 3H), 7.81 (s, 1H), 8.10 (s, 1H), 8.25 (s, 1H) |
| 3-897 | 1H-NMR (CDCl3) δ: 1.21 (t, 3H), 1.72-1.89 (m, 1H), 2.27 (q, 2H), 2.55-2.72 (m, 1H), 2.81-3.08 (m, 2H), 3.69 (d, 1H), 4.11 (d, 1H), |
| | 5.45-5.70 (m, 2H), 7.30-7.60 (m, 3H), 7.69 (s, 2H) |
| 3-898 | 1H-NMR (CDCl3) δ: 1.20 (t, 3H), 1.73-1.90 (m, 1H), 2.27 (q, 2H), 2.54-2.70 (m, 1H), 2.79-3.07 (m, 2H), 3.71 (d, 1H), 4.13 (d, 1H), |
| | 5.52 (q, 1H), 5.62-5.76 (m, 1H), 7.23-7.62 (m, 4H), 7.76-7.91 (m, 2H) |
| 3-899 | 1H-NMR (CDCl3) δ: 1.20 (t, 3H), 1.69-1.89 (m, 1H), 2.27 (q, 2H), 2.54-2.69 (m, 1H), 2.80-3.05 (m, 2H), 3.72 (d, 1H), 4.12 (d, 1H), |
| | 5.51 (q, 1H), 5.69-5.83 (m, 1H), 7.28-7.82 (m, 6H) |
| 3-900 | 1H-NMR (CDCl3) δ: 1.20 (t, 3H), 1.68-1.90 (m, 1H), 2.27 (q, 2H), 2.53-2.68 (m, 1H), 2.78-3.06 (m, 2H), 3.69 (d, 1H), 4.08 (d, 1H), |
| | 5.50 (q, 1H), 5.73-5.88 (m, 1H), 7.26-7.78 (m, 6H) |
| 3-901 | 1H-NMR (CDCl3) δ: 1.20 (t, 3H), 1.73-1.89 (m, 1H), 2.27 (q, 2H), 2.54-2.70 (m, 1H), 2.79-3.07 (m, 2H), 3.71 (d, 1H), 4.13 (d, 1H), |
| | 5.46-5.74 (m, 2H), 7.22-7.96 (m, 7H) |
| 3-902 | 1H-NMR (CDCl3) δ: 1.20 (t, 3H), 1.73-1.89 (m, 1H), 2.27 (q, 2H), 2.53-2.70 (m, 4H), 2.80-3.06 (m, 2H), 3.74 (d, 1H), 4.12 (d, 1H), |
| | 5.52 (q, 1H), 5.69 (d, 1H), 7.28-7.82 (m, 5H), 8.20 (s, 1H) |
| 3-903 | 1H-NMR (CDCl3) δ: 1.20 (t, 3H), 1.73-1.88 (m, 1H), 2.27 (q, 2H), 2.55-2.70 (m, 1H), 2.80-3.07 (m, 2H), 3.77 (d, 1H), 4.17 (d, 1H), |
| | 5.52 (q, 1H), 5.70 (d, 1H), 7.28-7.74 (m, 4H), 7.95-8.51 (m, 3H) |
| 3-904 | 1H-NMR (CDCl3) δ: 1.75-1.91 (m, 4H), 2.54-2.71 (m, 1H), 2.80-3.19 (m, 4H), 3.45 (s, 2H),, 5.51 (q, 1H), 6.03 (d, 1H), 7.17-7.79 |
| | (m, 6H) |
| 3-910 | 1H-NMR (CDCl3) δ: 1.58 (9H, s), 3.53 (1H, d), 3.92 (1H, d), 4.46 (1H, d), 4.91 (1H, d), 6.81 (1H, s), 7.42-7.44 (3H, m), 7.79-7.82 |
| | (2H, m), 8.02-8.08 (3H, m). |
| 3-912 | 1H-NMR (CDCl3) δ: 2.26 (3H, s), 3.55 (1H, d), 3.92 (1H, d), 4.47 (1H, d), 4.92 (1H, d), 7.38 (1H, s), 7.45 (2H, s), 7.52 (1H, d), |
| | 7.85 (2H, d), 8.05 (1H, d), 8.12 (1H, s), 8.23 (1H, s). |
| 3-913 | 1H-NMR (CDCl3) δ: 1.26-1.33 (3H, m), 2.44-2.52 (2H, m), 3.55 (1H, d), 3.92 (1H, d), 4.47 (1H, d), 4.92 (1H, d), 7.34 (1H, s), 7.45 |
| | (2H, s), 7.53 (1H, d), 7.85 (2H, t), 8.04 (1H, d), 8.12 (1H, s), 8.26 (1H, s). |
| 3-914 | 1H-NMR (CDCl3) δ: 0.87-0.90 (2H, m), 1.13-1.15 (2H, m), 1.56-1.61 (1H, m), 3.53 (1H, d), 3.91 (1H, d), 4.46 (1H, d), 4.91 (1H, d), |
| | 7.43-7.56 (3H, m), 7.78-7.83 (3H, m), 8.02 (1H, d), 8.10 (1H, s), 8.23 (1H, s). |
| 3-916 | 1H-NMR (CDCl3) δ: 1.16-1.19 (3H, m), 3.27-3.38 (2H, m), 3.51 (1H, d), 3.90 (1H, d), 4.45 (1H, d), 4.90 (1H, d), 5.05-5.07 (1H, m), |
| | 6.95 (1H, s), 7.41-7.42 (3H, m), 7.73-7.78 (2H, m), 7.90 (1H, s), 7.98-8.00 (1H, m), 8.07 (1H, s). |
| 3-917 | 1H-NMR (CDCl3) δ: 1.76-1.95 (m, 3H), 2.01-2.17 (m, 1H), 2.69-2.94 (m, 2H), 3.44 (s, 2H), 3.67 (d, 1H), 4.06 (d, 1H), 5.12-5.27 |
| | (m, 1H), 6.20-6.34 (m, 1H), 7.23-7.52 (m, 3H), 7.64 (s, 2H) |
| 3-918 | 1H-NMR (CDCl3) δ: 1.76-1.95 (m, 3H), 2.00-2.17 (m, 1H), 2.71-2.95 (m, 2H), 3.44 (s, 2H), 3.74 (d, 1H), 4.19 (d, 1H), 5.14-5.28 |
| | (m, 1H), 6.24-6.38 (m, 1H), 7.25-7.57 (m, 3H), 7.97 (s, 1H), 8.09 (s, 2H) |
| 3-919 | 1H-NMR (CDCl3) δ: 1.73-1.97 (m, 3H), 2.01-2.14 (m, 1H), 2.28 (s, 3H), 2.69-2.95 (m, 2H), 3.49 (s, 2H), 3.72 (d, 1H), 4.18 (d, 1H), |
| | 5.16-5.30 (m, 1H), 7.15-7.53 (m, 4H), 7.96 (s, 1H), 8.08 (s, 2H) |
| 3-920 | 1H-NMR (CDCl3) δ: 1.57 (9H, s), 3.55 (1H, d), 3.93 (1H, d), 4.48 (1H, d), 4.93 (1H, d), 6.94 (1H, s), 7.48-7.51 (3H, m), 7.68 |
| | (1H, d), 7.91-7.98 (2H, m), 8.09-8.13 (2H, m). |
| 3-921 | 1H-NMR (CDCl3) δ: 3.53 (1H, d, J = 17.6 Hz), 3.91 (1H, d, J = 17.6 Hz), 4.00 (2H, br s), 4.45 (1H, d), 4.89 (1H, d), |
| | 6.96-7.00 (2H, m), 7.45 (2H, s), 7.62 (1H, d), 7.72 (1H, d), 7.95 (1H, d), 8.03 (1H, s). |
| 3-922 | 1H-NMR (CDCl3) δ: 1.49 (9H, s), 1.79-1.84 (4H, m), 2.80-2.82 (2H, m), 3.42 (1H, d), 3.78 (1H, d), 4.41 (1H, d), 4.84-4.87 (3H, m), |
| | 7.41-7.45 (3H, m), 7.57-7.64 (2H, m). |
| 3-923 | 1H-NMR (CDCl3) δ: 1.75-1.93 (m, 1H), 2.53-2.73 (m, 1H), 2.81-3.21 (m, 4H), 3.74 (d, 1H), 4.13 (d, 1H), 5.52 (q, 1H), 5.89-6.09 |
| | (m, 1H), 7.21-7.91 (m, 7H) |
| 3-924 | 1H-NMR (CDCl3) δ: 2.07 (s, 3H), 2.08-2.20 (m, 1H), 2.45-2.59 (m, 1H), 2.82-2.97 (m, 1H), 3.04-3.20 (m, 1H), 3.74 (d, 1H), 4.14 |
| | (d, 1H), 6.18 (dd, 1H), 7.41-7.91 (m, 7H) |
| 3-925 | 1H-NMR (CDCl3) δ: 1.56 (bs, 2H), 1.63-1.80 (m, 1H), 2.45-2.60 (m, 1H), 2.74-3.05 (m, 2H), 3.74 (d, 1H), 4.14 (d, 1H), 4.37 (t, 1H), |
| | 7.34-7.94 (m, 7H) |
| 3-926 | 1H-NMR (CDCl3) δ: 1.72-1.89 (m, 1H), 2.04 (s, 3H), 2.53-2.68 (m, 1H), 2.77-3.06 (m, 2H), 3.74 (d, 1H), 4.13 (d, 1H), 5.49 (q, 1H), |
| | 5.70-5.86 (m, 1H), 7.27-7.94 (m, 7H) |
| 3-927 | 1H-NMR (CDCl3) δ: 1.20 (t, 3H), 1.73-1.88 (m, 1H), 2.26 (q, 2H), 2.54-2.69 (m, 1H), 2.78-3.07 (m, 2H), 3.74 (d, 1H), 4.13 (d, 1H), |
| | 5.52 (q, 1H), 5.62-5.76 (m, 1H), 7.27-7.93 (m, 7H) |
| 3-928 | 1H-NMR (CDCl3) δ: 0.69-0.82 (m, 2H), 0.96-1.08 (m, 2H), 1.31-1.43 (m, 1H), 1.75-1.91 (m, 1H), 2.52-2.68 (m, 1H), 2.77-3.06 (m, 2H), |
| | 3.74 (d, 1H), 4.14 (d, 1H), 5.51 (q, 1H), 5.84-5.98 (m, 1H), 7.28-7.92 (m, 7H) |
| 3-929 | 1H-NMR (CDCl3) δ: 0.16-0.29 (m, 2H), 0.54-0.66 (m, 2H), 0.89-1.06 (m, 1H), 1.74-1.90 (m, 1H), 2.22 (d, 2H), 2.58-2.73 (m, 1H), |
| | 2.81-3.09 (m, 2H), 3.74 (d, 1H), 4.14 (d, 1H), 5.55 (q, 1H), 6.01-6.13 (m, 1H), 7.29-7.92 (m, 7H) |
| 3-930 | 1H-NMR (CDCl3) δ: 1.72-1.90 (m, 1H), 2.56-2.70 (m, 1H), 2.80-3.11 (m, 4H), 3.74 (d, 1H), 4.13 (d, 1H), 5.18-5.28 (m, 2H), 5.50 |
| | (q, 1H), 5.78-6.05 (m, 2H), 7.27-7.91 (m, 7H) |
| 3-932 | 1H-NMR (CDCl3) δ: 1.78-1.93 (m, 1H), 2.13 (s, 3H), 2.58-2.72 (m, 1H), 2.83-3.11 (m, 2H), 3.27 (s, 2H), 3.75 (d, 1H), 4.14 (d, 1H), |
| | 5.53 (q, 1H), 7.01-7.12 (m, 1H), 7.29-7.91 (m, 7H) |
| 3-933 | 1H-NMR (CDCl3) δ: 1.79-1.96 (m, 1H), 2.53-2.69 (m, 1H), 2.80-3.15 (m, 5H), 3.73 (d, 1H), 3.90 (s, 2H), 4.12 (d, 1H), 5.47 (q, 1H), |
| | 6.70-6.80 (m, 1H), 7.30-7.91 (m, 7H) |
| 3-934 | 1H-NMR (CDCl3) δ: 0.52-0.62 (m, 2H), 0.67-0.78 (m, 2H), 1.73-1.90 (m, 1H), 2.37-2.49 (m, 1H), 2.59-2.74 (m, 1H), 2.80-3.06 (m, 2H), |
| | 3.74 (d, 1H), 4.14 (d, 1H), 4.83 (s, 1H), 5.12-5.23 (m, 1H), 5.42 (q, 1H), 7.33-7.92 (m, 7H) |
| 4-1 | 1H-NMR ([D6]-DMSO) 4.30-4.43 (2H, AB spectrum); 6.34 (br. s, 2H); 7.61 (d, 2H); 7.66 (dd, 1H); 7.74 (m, 1H); 7.75 (br. s, 1H); 7.91 |
| | (dd, 1H) |
| 4-3 | 1H-NMR (CD3CN) 1.21 (t, 3H); 2.52 (q, 2H); 4.01 (d, 1H); 4.27 (d, 1H); 7.56 (m, 1H); 7.60 (m, 2H); 7.72 (dd, 1H); 7.79 (m, 1H); 8.11 |
| | (dd, 1H); 8.90 (br. s, 1H) |
| 4-6 | 1H-NMR (CD3CN) 1.24 (d, 6H); 2.76 (m, 1H); 4.04 (d, 1H); 4.27 (d, 1H); 7.56 (m, 1H); 7.60 (m, 2H); 7.72 (dd, 1H); 7.80 (m, 1H); 8.09 |
| | (dd, 1H); 8.88 (br. s, 1H) |
| 4-11 | 1H-NMR d ([D₆]-DMSO) 4.34-4.49 (2H, AB spectrum); 7.62 (m, 2H); 7.75 (m, 1H); 7.83 (m, 1H); 8.05-8.07 (m, 2H); 12.5 |
| | (br. s, 1H) |
| 4-17 | 1H-NMR d ([D₆]-DMSO) 3.78 (s, 3H); 4.32-4.64 (2H, AB spectrum); 7.62 (br. s, 2H); 7.74-7.77 (m, 2H); 7.96 (br. s, 1H); 8.13 |
| | (d, 1H); 10.72 (s, 1H) |
| 4-19 | 1H-NMR d (CD₃CN) 1.18 (d, 12 H); 3.06 (m, 2H); 4.03 (d, 1H); 4.29 (d, 1H); 7.57 (m, 1H); 7.61 (m, 2H); 7.64 (dd, 1H); 7.82 |
| | (dd, 1H); 7.96 (m, 1H) |
| 4-41 | 1H-NMR ([D6]-DMSO) 3.96-4.27 (2H, AB spectrum); 5.04 (br. s, 2H); 7.47 (dd, 1H); 7.55 (m, 1H); 7.60 (br. s, 2H); 7.89 (dd, 1H); |
| | 8.03 (m, 1H) |
| 4-43 | 1H-NMR (CD3CN) 1.26 (d, 6H); 2.78 (m, 1H); 3.98 (d, 1H); 4.24 (d, 1H); 7.55 (m, 1H); 7.60-7.63 (m, 3H); 7.96 (dd, 1H); 8.37 (m, 1H); |
| | 8.88 (br. s, 1H) |
| 4-44 | 1H-NMR (CD3CN) 4.03 (d, 1H); 4.27 (d 1H); 7.58 (m, 1H); 7.63 (br. s, 2H); 7.30 (dd, 1H); 8.07 (dd, 1H); 8.27 (m, 1H) |
| 4-47 | 1H-NMR ([D6]-DMSO) 1.18 (d, 6H); 3.9 (m, 1H); 4.28-4.31 (2H, AB spectrum); 6.98 (m, 1H); 7.63 (br. s, 2H); 7.70-7.73 (m, 3H); |
| | 7.98 (dd, 1H); 8.62 (m, 1H) |
| 4-48 | 1H-NMR (CD3CN) 3.83 (s, 3H); 4.01 (d, 1H); 4.27 (d, 1H); 7.58 (m, 1H); 7.62-7.65 (m, 3H); 7.98 (dd, 1H); 8.38 (m, 1H); 8.6 (br. s, 1H) |
| 4-49 | 1H-NMR (CD3CN) 3.95 (d, 1H); 4.22 (d, 1H); 7.26-7.33 (m, 3H); 7.42-7.49 (m, 2H); 7.54 (m, 1H); 7.60 (br. s, 2H); 7.65 (d, 1H); 8.00 |
| | (dd, 1H); 8.38 (m, 1H); 8.88 (br. s, 1H) |
| 4-50 | 1H-NMR ([D6]-DMSO) 3.78 (s, 3H); 4.26-4.43 (2H, AB spectrum); 5.34 (br. s, 2H), 7.32 (dd, 1H); 7.62 (br. s, 2H); 7.65 (s, 1H); |
| | 7.73-7.75 (m, 2H) |
| 4-54 | 1H-NMR (CD3CN) 3.78 (s, 3H); 3.97 (d, 1H); 4.23 (d, 1H); 7.29 (dd, 1H); 7.54 (m, 1H); 7.60 (br. s, 2H); 7.72 (dd, 1H); 7.89 (br. s, 1H) |
| 4-56 | 1H-NMR (CD3CN) 1.25 (d, 6H); 2.71 (m, 1H); 3.94 (s, 3H); 3.97 (d, 1H); 4.23 (d, 1H); 7.46 (d, 1H); 7.54 (m, 1H); 7.62 (br. s, 2H); |
| | 7.79 (dd, 1H); 8.12 (br. s, 1H); 8.4 (br. s, 1H) |
| 4-57 | 1H-NMR (CD3CN) 3.98 (d, 2H); 4.01 (s, 3H); 4.24 (d, 2H); 7.54-7.56 (m, 2H); 7.61 (br. s, 2H); 7.87 (dd, 1H); 8.04 (br. s, 1H); |
| | 9.51 (br. s, 1H) |
| 4-60 | 1H-NMR ([D7]-DMF) 1.23 (d, 6H); 3.98 (m, 1H); 4.01 (s, 3H); 4.36-4.49 (2H, AB spectrum); 7.62 (br. d, 1H); 7.65 (br. d, 1H); 7.74 |
| | (br. s, 2H); 7.79 (m, 1H); 7.89 (dd, 1H); 8.57 (br. s, 1H); 9.48 (br. s, 1H) |
| 4-61 | 1H-NMR ([D7]-DMF) 3.78 (s, 3H); 4.03 (s, 3H); 4.41-4.54 (2H, AB spectrum); 7.71 (br. d, 1H); 7.76 (br. s, 2H); 7.79 (m, 1H); 7.89 |
| | (dd, 1H); 8.29 (br. s, 1H); 10.0 (br. s, 1H) |
| 6-70 | 1H-NMR (CDCl3) δ: 1.99-2.25 (4H, m), 2.50-2.66 (1H, m), 2.86-3.05 (1H, m), 3.09-3.28 (1H, m), 6.22 (1H, dd, J = 7.2, 4.5 Hz), |
| | 7.56 (1H, d), 7.75 (1H, d), 7.79 (1H, s), 10.02 (1H, s) |
| 6-175 | 1H-NMR (CDCl3) δ: 1.76-2.20 (7H, m), 2.73-3.04 (2H, m), 6.03 (1H, t), 7.65 (1H, s), 7.68 (1H, d), 7.67 (1H, s), 9.98 (1H, s) |
| 7-446 | 1H-NMR (CDCl3) δ: 0.17 (9H, s), 1.48 (9H, s), 1.75-1.82 (3H, m), 2.02-2.05 (1H, m), 2.78-2.80 (2H, m), 3.52 (2H, d), 4.74-4.81 |
| | (2H, m), 7.35-7.41 (2H, m), 7.47 (1H, s), 7.59 (1H, br s). |
| 7-470 | 1H-NMR (CDCl3) δ: 0.20 (9H, s), 1.56 (9H, s), 3.56 (2H, d), 6.88 (1H, s), 7.40 (1H, t), 7.55 (1H, d), 7.73-7.79 (3H, m), 7.96 |
| | (1H, s), 8.07 (1H, br s). |
| 7-480 | 1H-NMR (CDCl3) δ: 0.21 (9H, s), 1.56 (9H, s), 3.58 (2H, d), 6.81 (1H, s), 7.36 (1H, d), 7.73-7.76 (4H, m), 8.02 (1H, s), 8.07 |
| | (1H, s). |

### BIOLOGICAL TEST EXAMPLE 1

### Test on Spodoptera litura Larvae

Solvent: Dimethylformamide, 3 parts by weight
Emulsifier: Polyoxyethylene alkylphenyl ether, 1 part by weight

To prepare the test solution 1 part by weight of the active compound is mixed with the above-described amount of the solvent which contains the above-described amount of emulsifier. The resulting mixture is diluted with water to a predetermined concentration.

Leaves of sweet potato were immersed in the test solution. The leaves are air-dried and placed in a petri dish (9 cm diameter). 10 Spodoptera litura third instar larvae were released in the petri dish, which was then stored in a constant temperature room (25° C.). More sweet potato leaves were added after 2 and 4 days. 7 days after the release of the larvae mortality was calculated by counting the number of dead larvae. 100% means that all the larvae have been killed; 0% means that none of the larvae have been killed. In the present test, an average value was taken from the results obtained from 2 petri dishes for 1 group.

Compounds having the example nos. 1-50 and 1-58, known from WO2005/085216, showed a control efficacy with 100% mortality at an active compound concentration of 100 ppm.

The following compounds showed a control efficacy with 100% mortality at an active compound concentration of 100 ppm:

Example Nos.: 1-2, 1-10, 1-38, 1-44, 1-54, 1-56, 1-67, 1-69, 1-79, 1-80, 1-114, 1-121, 1-125, 1-126, 1-127, 1-129, 1-131, 1-132, 1-150, 1-211, 1-213, 1-223, 1-232, 1-235, 1-250, 1-255, 1-259, 1-264, 1-271, 1-272, 1-273, 1-295, 3-1, 3-3, 3-4, 3-6, 3-7, 3-8, 3-9, 3-23, 3-32, 3-39, 3-40, 3-42, 3-43, 3-44, 3-45, 3-48, 3-55, 3-57, 3-61, 3-73, 3-74, 3-81, 3-82, 3-88, 3-89, 3-90, 3-91, 3-92, 3-93, 3-95, 3-97, 3-98, 3-104, 3-106, 3-112, 3-140, 3-142, 3-143, 3-145, 3-166, 3-188, 3-191, 3-193, 3-211, 3-213, 3-217, 3-218, 3-219, 3-220, 3-221, 3-230, 3-235, 3-236, 3-238, 3-239, 3-241, 3-251, 3-253, 3-254, 3-256, 3-257, 3-258, 3-259, 3-261, 3-270, 3-302, 3-303, 3-304, 3-305, 3-306, 3-307, 3-320, 3-322, 3-323, 3-326, 3-355, 3-358, 3-361, 3-547, 3-549, 3-551, 3-637, 3-638, 3-641, 3-642, 3-643, 3-644, 3-645, 3-646, 3-649, 3-650, 3-652, 3-656, 3-659, 3-661, 3-663, 3-664, 3-782, 3-785, 3-829, 3-831, 3-832, 3-833, 3-835, 3-836, 3-837, 3-838, 3-839, 3-840, 3-841, 3-842, 3-843, 3-844, 3-845, 3-846, 3-847, 3-848, 3-849, 3-850, 3-851, 3-852, 3-853, 3-854, 3-855, 3-856, 3-858, 3-859, 3-862, 3-863, 3-864, 3-865, 3-866, 3-867, 3-868, 3-869, 3-870, 3-871, 3-872, 3-873, 3-874, 3-875, 3-876, 3-877, 3-878, 3-879, 3-880, 3-881, 3-882, 3-883, 3-884, 3-885, 3-886, 3-887, 3-888, 3-889, 3-890, 3-891, 3-892, 3-893, 3-894, 3-896, 3-897, 3-898, 3-899, 3-900, 3-902, 3-904, 3-916, 3-917, 3-918, 3-919.

### Biological Test Example 2

### Test on Tetranychus urticae (Spray Test)

The test solution has been prepared as described in biological test example 1. 50 to 100 adult Tetranychus urticae were placed on pinto bean leaves at true leaf stage having two main leaves that had been grown in a pot (6 cm diameter). After 1 day, a generous amount of the test solution which has been diluted with water to the predetermined concentration was sprayed thereto using a spray gun.

After keeping the pot in a green house for 7 days, acaricidal ratio was calculated. 100% means that all the mites have been killed; 0% means that none of the mites have been killed.

The compound having the example no. 1-58, known from WO2005/085216, showed a control efficacy with at least 98% mortality at an active compound concentration of 100 ppm.

The following compounds showed a control efficacy with at least 98% mortality at an active compound concentration of 100 ppm:

Example Nos.: 1-38, 1-44, 1-114, 1-127, 1-129, 1-132, 1-232, 1-235, 1-259, 1-264, 1-273, 3-3, 3-4, 3-6, 3-7, 3-8, 3-9, 3-23, 3-32, 3-39, 3-40, 3-42, 3-43, 3-44, 3-45, 3-48, 3-55, 3-57, 3-61, 3-70, 3-73, 3-74, 3-81, 3-82, 3-88, 3-89, 3-90, 3-91, 3-92, 3-93, 3-95, 3-97, 3-98, 3-106, 3-112, 3-142, 3-143, 3-145, 3-166, 3-188, 3-191, 3-193, 3-213, 3-216, 3-217, 3-218, 3-219, 3-220, 3-221, 3-230, 3-235, 3-236, 3-238, 3-239, 3-253, 3-254, 3-256, 3-257, 3-258, 3-259, 3-261, 3-270, 3-302, 3-303, 3-304, 3-305, 3-306, 3-307, 3-323, 3-326, 3-355, 3-358, 3-361, 3-549, 3-551, 3-642, 3-644, 3-661, 3-829, 3-832, 3-833, 3-835, 3-836, 3-838, 3-839, 3-840, 3-841, 3-842, 3-843, 3-844, 3-845, 3-846, 3-847, 3-848, 3-849, 3-850, 3-851, 3-852, 3-853, 3-854, 3-855, 3-856, 3-859, 3-862, 3-863, 3-864, 3-865, 3-866, 3-867, 3-868, 3-869, 3-870, 3-871, 3-872, 3-873, 3-874, 3-875, 3-876, 3-877, 3-878, 3-880, 3-881, 3-883, 3-884, 3-885, 3-886, 3-887, 3-888, 3-889, 3-890, 3-891, 3-892, 3-893, 3-895, 3-896, 3-897, 3-898, 3-899, 3-900, 3-902, 3-903, 3-917, 3-918, 3-919.

### Biological Test Example 3

### Test on Aulacophora femoralis (Spray Application)

The test solution has been prepared as described in biological test example 1.

Cucumber leaves were immersed in a test solution that had been diluted to a predetermined concentration with water. The leaves were air-dried and then put in a plastic cup containing sterilized black soil. 5 Aulacophora femoralis second instar larvae were released in the cup. 7 days later, mortality was calculated by counting the number of dead larvae. 100% means that all larvae have been killed; 0% means that none of the larvae have been killed.

Compounds having the example nos. 1-50, 1-52, and 1-58, known from WO2005/085216, showed a control efficacy with 100% mortality at an active compound concentration of 100 ppm.

The following compounds showed a control efficacy with 100% mortality at an active compound concentration of 100 ppm:

Example Nos.: 1-2, 1-38, 1-44, 1-54, 1-61, 1-64, 1-80, 1-114, 1-115, 1-125, 1-127, 1-129, 1-131, 1-132, 1-142, 1-150, 1-151, 1-169, 1-221, 1-232, 1-251, 1-259, 1-264, 1-272, 1-273, 3-1, 3-3, 3-4, 3-6, 3-7, 3-8, 3-9, 3-23, 3-32, 3-39, 3-40, 3-42, 3-43, 3-44, 3-45, 3-48, 3-55, 3-57, 3-61, 3-73, 3-74, 3-81, 3-82, 3-88, 3-89, 3-90, 3-91, 3-92, 3-93, 3-95, 3-97, 3-98, 3-106, 3-112, 3-142, 3-143, 3-145, 3-166, 3-188, 3-191, 3-193, 3-211, 3-213, 3-217, 3-218, 3-220, 3-221, 3-230, 3-235, 3-236, 3-238, 3-239, 3-241, 3-253, 3-254, 3-256, 3-257, 3-258, 3-259, 3-261, 3-270, 3-302, 3-303, 3-304, 3-305, 3-306, 3-307, 3-323, 3-326, 3-358, 3-361, 3-547, 3-549, 3-551, 3-637, 3-638, 3-640, 3-642, 3-643, 3-644, 3-645, 3-646, 3-651, 3-652, 3-659, 3-663, 3-664, 3-779, 3-785, 3-829, 3-831, 3-832, 3-833, 3-835, 3-836, 3-837, 3-838, 3-839, 3-841, 3-842, 3-843, 3-844, 3-845, 3-846, 3-847, 3-848, 3-849, 3-850, 3-851, 3-852, 3-853, 3-854, 3-855, 3-856, 3-859, 3-862, 3-863, 3-864, 3-865, 3-866, 3-867, 3-868, 3-869, 3-870, 3-871, 3-872, 3-873, 3-874, 3-875, 3-876, 3-877, 3-878, 3-880, 3-881, 3-882, 3-883, 3-884, 3-885, 3-886, 3-887, 3-888, 3-889, 3-890, 3-891, 3-892, 3-893, 3-894, 3-895, 3-896, 3-897, 3-898, 3-899, 3-900, 3-901, 3-902, 3-903, 3-904, 3-917, 3-918, 3-919.

### Biological Test Example 4

### Musca domestica—Test

Solvent: dimethyl sulfoxide

To produce a suitable preparation of active compound, 10 mg of active compound are dissolved in 0.5 ml solvent, and the concentrate is diluted with water to the desired concentration. Prior to the assay, a piece or kitchen sponge is soaked with a mixture of sugar and compound solution and placed into a container. 10 adults (Musca domestica) are placed into the container and closed with a perforated lid. After 2 days, mortality in % is determined. 100% means that all the flies have been killed; 0% means that none of the flies have been killed.

Compounds having the example nos. 1-50, 1-52, and 1-58, known from WO2005/085216, showed good acitivity of ≧80% at application rate of 100 ppm.

In this test for example, the following compounds from the preparation examples showed good activity of ≧80% at application rate of 100 ppm:

Example Nos.: 1-2, 1-10, 1-38, 1-44, 1-54, 1-56, 1-61, 1-64, 1-80, 1-114, 1-125, 1-126, 1-129, 1-131, 1-132, 1-142, 1-211, 1-213, 1-232, 1-235, 1-255, 1-264, 1-271, 1-272, 3-3, 3-7, 3-9, 3-23, 3-32, 3-39, 3-40, 3-42, 3-43, 3-44, 3-45, 3-48, 3-55, 3-90, 3-91, 3-92, 3-93, 3-95, 3-106, 3-112, 3-213, 3-217, 3-218, 3-221, 3-230, 3-235, 3-236, 3-239, 3-253, 3-256, 3-257, 3-258, 3-259, 3-261, 3-270, 3-302, 3-304, 3-305, 3-320, 3-323, 3-326, 3-637, 3-638, 3-640, 3-641, 3-642, 3-643, 3-644, 3-645, 3-646, 3-651, 3-652, 3-656, 3-785, 3-829, 3-832, 3-833, 3-835, 3-836, 3-838, 3-844, 3-845, 3-846, 3-307, 3-847, 3-848, 3-851, 3-57, 3-849, 3-89, 3-303, 3-852, 3-4, 3-878, 3-8, 3-859, 3-862, 3-863, 3-864, 3-865, 3-74, 3-254, 3-917, 3-918, 3-880, 3-98, 3-881, 3-853, 3-868, 3-73, 3-897, 3-898, 3-97, 3-81, 3-872, 3-361, 3-355, 3-358.

### Biological Test Example 5

### Lucillia cuprina—Test

Solvent: dimethyl sulfoxide

To produce a suitable preparation of active compound, 10 mg of active compound are dissolved in 0.5 ml solvent, and the concentrate is diluted with water to the desired concentration. Approximately 20-30 (Lucilia cuprina larvae) are transferred into a test tube containing 1 cm³ of minced horse meat and 0.5 ml aqueous dilution of test compound. After 2 days, mortality in % is determined. 100% means that all the larvae have been killed; 0% means that none of the larvae have been killed.

Compounds having the example nos. 1-50, 1-52 and 1-58, known from WO2005/085216, showed good activity of ≧80% at application rate of 100 ppm.

In this test for example, the following compounds from the preparation examples showed good activity of ≧80% at application rate of 100 ppm:

Example Nos.: 1-2, 1-10, 1-38, 1-44, 1-54, 1-56, 1-61, 1-64, 1-67, 1-69, 1-79, 1-80, 1-114, 1-116, 1-121, 1-125, 1-126, 1-129, 1-131, 1-132, 1-142, 1-150, 1-151, 1-155, 1-164, 1-211, 1-213, 1-232, 1-235, 1-241, 1-255, 1-264, 1-270, 1-271, 1-272, 3-3, 3-7, 3-9, 3-23, 3-32, 3-39, 3-40, 3-42, 3-43, 3-44, 3-45, 3-48, 3-55, 3-70, 3-90, 3-91, 3-92, 3-93, 3-95, 3-106, 3-112, 3-213, 3-216, 3-217, 3-218, 3-219, 3-221, 3-230, 3-235, 3-236, 3-239, 3-241, 3-253, 3-256, 3-257, 3-258, 3-259, 3-261, 3-270, 3-302, 3-304, 3-305, 3-320, 3-323, 3-326, 3-344, 3-637, 3-638, 3-640, 3-641, 3-642, 3-643, 3-644, 3-645, 3-646, 3-651, 3-652, 3-656, 3-659, 3-660, 3-661A, 3-663, 3-664, 3-785, 3-828, 3-829, 3-831, 3-832, 3-833, 3-835, 3-836, 3-838, 3-844, 3-845, 3-846, 3-307, 3-847, 3-848, 3-851, 3-57, 3-849, 3-89, 3-303, 3-852, 3-4, 3-878, 3-8, 3-859, 3-862, 3-863, 3-864, 3-865, 3-74, 3-254, 3-917, 3-918, 3-880, 3-98, 3-881, 3-853, 3-868, 3-73, 3-897, 3-898, 3-97, 3-81, 3-872, 3-361, 3-355, 3-358.

### Biological Test Example 6

### Ctenocephalides felis—Test

Solvent: dimethyl sulfoxide

To produce a suitable preparation of active compound, 10 mg of active compound are dissolved in 0.5 ml solvent, and the concentrate is diluted with cattle blood to the desired concentration. Approximately 10 to 15 adult unfed (Ctenocepahlides felis) are placed in flea chambers. The blood chamber, are sealed with parafilm on the bottom are filled with cattle blood supplied with compound solution and placed on top of the flea chamber, so that the fleas are able to suck the blood. The blood chamber is heated to 37° C. whereas the flea chamber is kept at room temperature. After 2 days, mortality in % is determined. 100% means that all the fleas have been killed; 0% means that none of the fleas have been killed.

Compounds having the example nos. 1-50, 1-52 and 1-58, known from WO2005/085216, showed good activity of ≧80% at application rate of 100 ppm.

In this test for example, the following compounds from the preparation examples showed good activity of ≧80% at application rate of 100 ppm:

Example Nos.: 1-2, 1-10, 1-38, 1-44, 1-54, 1-56, 1-61, 1-64, 1-67, 1-80, 1-114, 1-115, 1-121, 1-125, 1-126, -129, 1-131, 1-132, 1-150, 1-164, 1-232, 1-235, 1-255, 1-264, 1-271, 1-272, 3-3, 3-7, 3-9, 3-23, 3-32, 3-39, 3-40, 3-42, 3-43, 3-44, 3-45, 3-48, 3-55, 3-90, 3-91, 3-92, 3-93, 3-95, 3-106, 3-112, 3-213, 3-216, 3-217, 3-218, 3-221, 3-230, 3-235, 3-236, 3-239, 3-241, 3-253, 3-256, 3-257, 3-258, 3-259, 3-261, 3-270, 3-302, 3-304, 3-305, 3-320, 3-323, 3-326, 3-637, 3-638, 3-640, 3-641, 3-642, 3-643, 3-644, 3-645, 3-646, 3-651, 3-652, 3-656, 3-663, 3-785, 3-829, 3-831, 3-832, 3-833, 3-835, 3-836, 3-838, 3-844, 3-845, 3-846, 1-69, 3-307, 3-847, 3-848, 3-851, 3-57, 3-849, 3-89, 3-303, 3-852, 3-4, 3-878, 3-8, 3-859, 3-862, 3-863, 3-864, 3-865, 3-74, 3-254, 3-917, 3-918, 3-880, 3-98, 3-881, 3-853, 3-868, 3-73, 3-897, 3-898, 3-97, 3-81, 3-872, 3-361, 3-355, 3-358.

### Biological Test Example 7

### Boophilus microplus—Test (Injection)

Solvent: dimethyl sulfoxide

To produce a suitable preparation of active compound, 10 mg of active compound are dissolved in 0.5 ml solvent, and the concentrate is diluted with solvent to the desired concentration. Five adult engorged female ticks (Boophilus microplus) are injected with compound solution into the abdomen. Ticks are transferred into replica plates and incubated in a climate chamber for a period of time. Egg deposition of fertile eggs is monitored. After 7 days, mortality in % is determined. 100% means that all eggs are infertile; 0% means that all eggs are fertile.

Compounds having the example nos. 1-50, 1-52 and 1-58, known from WO2005/085216, showed good activity of ≧80% at application rate of 20 μg/animal.

In this test for example, the following compounds from the preparation examples showed good activity of ≧80% at application rate of 20 μg/animal:

Example Nos.:1-2, 1-10, 1-38, 1-44, 1-54, 1-56, 1-61, 1-64, 1-67, 1-69, 1-79, 1-80, 1-114, 1-115, 1-116, 1-121, 1-125, 1-126, 1-129, 1-131, 1-132, 1-142, 1-150, 1-151, 1-164, 1-169, 1-175, 1-211, 1-213, 1-232, 1-235, 1-241, 1-255, 1-264, 1-270, 1-271, 1-272, 3-3, 3-7, 3-9, 3-23, 3-32, 3-39, 3-40, 3-42, 3-43, 3-44, 3-45, 3-48, 3-55, 3-70, 3-90, 3-91, 3-92, 3-93, 3-95, 3-106, 3-112, 3-137, 3-138, 3-211, 3-213, 3-216, 3-217, 3-218, 3-219, 3-221, 3-230, 3-235, 3-236, 3-239, 3-241, 3-253, 3-254, 3-256, 3-257, 3-258, 3-259, 3-261, 3-270, 3-302, 3-304, 3-305, 3-320, 3-323, 3-326, 3-344, 3-637, 3-638, 3-640, 3-641, 3-642, 3-643, 3-644, 3-645, 3-646, 3-651, 3-652, 3-656, 3-659, 3-660, 3-661A, 3-663, 3-664, 3-785, 3-828, 3-829, 3-832, 3-833, 3-835, 3-836, 3-838, 3-844, 3-845, 3-846, 3-307, 3-847, 3-848, 3-851, 3-57, 3-849, 3-89, 3-303, 3-852, 3-4, 3-878, 3-8, 3-859, 3-862, 3-863, 3-864, 3-865, 3-74, 3-254, 3-917, 3-918, 3-880, 3-98, 3-881, 3-853, 3-868, 3-73, 3-897, 3-898, 3-97, 3-81, 3-872, 3-361, 3-355, 3-358.

### Biological Test Example 8

### Boophilus microplus—Test (Dip)

Solvent: dimethyl sulfoxide

To produce a suitable preparation of active compound, 10 mg of active compound are dissolved in 0.5 ml solvent, and the concentrate is diluted with water to the desired concentration. Eight to ten adult engorged female Boophilus microplus ticks are placed in perforated plastic beakers and immersed in aqueous compound solution for one minute. Ticks are transferred to a filter paper in a plastic tray. Egg deposition of fertile eggs is monitored after. After 7 days, mortality in % is determined. 100% means that all the ticks have been killed; 0% means that none of the ticks have been killed.

In this test for example, the following compounds from the preparation examples showed good activity of ≧80% at application rate of 100 ppm:

Example Nos.: 1-2, 1-38, 1-114, 3-3, 3-4, 3-7, 3-8, 3-9, 3-23, 3-39, 3-40, 3-42, 3-43, 3-44, 3-45, 3-48, 3-55, 3-57, 3-74, 3-89, 3-90, 3-91, 3-92, 3-93, 3-98, 3-106, 3-112, 3-213, 3-217, 3-218, 3-221, 3-235, 3-239, 3-253, 3-254, 3-256, 3-257, 3-258, 3-259, 3-261, 3-270, 3-305, 3-307, 3-323, 3-326, 3-358, 3-361, 3-785, 3-835, 3-836, 3-838, 3-844, 3-845, 3-846, 3-847, 3-849, 3-851, 3-852, 3-863, 3-864, 3-865, 3-878, 3-880, 3-881, 3-917, 3-307, 3-847, 3-851, 3-57, 3-849, 3-89, 3-852, 3-4, 3-878, 3-8, 3-863, 3-864, 3-865, 3-74, 3-254, 3-917, 3-880, 3-98, 3-881, 3-853, 3-868, 3-73, 3-897, 3-898, 3-97, 3-81, 3-872, 3-361, 3-358, 3-870, 3-887, 3-890, 3-874, 3-875, 3-876, 3-877, 3-142, 3-145.

### Biological Test Example 9

### Amblyomma Hebraeum—Test

Solvent: dimethyl sulfoxide

To produce a suitable preparation of active compound, 10 mg active compound is mixed with 0.5 ml solvent, and the concentrate is diluted with containing solvent to the desired concentration. Nymphs of the tick Amblyomma hebraeum are placed in perforated plastic beakers and immersed in aqueous compound solution for one minute. Ticks are transferred to a filter paper in a Petri dish and incubated in a climate chamber for 42 days. After the specified period of time, mortality in % is determined. 100% means that all the ticks have been killed; 0% means that none of the ticks have been killed. In this test for example, the following compounds from the preparation examples showed good activity of ≧80% at application rate of 100 ppm:

Example Nos.: 3-9, 3-39, 3-40, 3-90, 3-91, 3-93, 3-832, 3-833, 3-835, 3-836, 3-838

### Biological Test Example 10

### Phaedon cochleariae—Test (Spray Application)

Solvent: 78.0 parts by weight of acetone
1.5 parts by weight of dimethylformamide
   Emulsifier: 0.5 parts by weight of alkylaryl polyglycolether

To produce a suitable preparation of active compound, 1 part by weight of active compound is mixed with the stated amount of solvent and emulsifier, and the concentrate is diluted with emulsifier-containing water to the desired concentration. Chinese cabbage (Brassica pekinesis) leaf-disks are sprayed with a preparation of the active ingredient of the desired concentration. Once dry, the leaf disks are infested with mustard beetle larvae (Phaedon cochleariae).

After 7 days, mortality in % is determined. 100% means that all beetle larvae have been killed and 0% means that none of the beetle larvae have been killed. In this test for example, the following compounds from the preparation examples showed good activity of 80% at application rate of 500 g/ha:

Example Nos.: 1-2, 1-209, 1-224, 4-3, 4-6,4-11, 4-17, 4-19, 4-41, 4-44, 4-48, 4-56, 4-57

### BIOLOGICAL TEST EXAMPLE 11

### Spodoptera frugiperda—Test (Spray Application)

Solvent: 78.0 parts by weight acetone
1.5 parts by weight dimethylformamide
   Wetting agent 0.5 parts by weight alkylarylpolyglcolether

To produce a suitable preparation of the active compound, 1 part by weight of active compound is mixed with the stated amount of solvent and emulsifier, and the concentrate is dilutes with emulsifier-containing water to the desired concentration.

Maize (Zea mais) leaf sections are sprayed with a preparation of the active ingredient of the desired concentration. Once dry, the leaf sections are infested with fall armyworm larvae (Spodoptera frugiperda). After 7 days, mortality in % is determined. 100% means that all caterpillars have been killed and 0% means that none of the caterpillars have been killed. In this test for example, the following compounds from the preparation examples showed good activity of ≧80% at application rate of 500 g/ha:

Example Nos.: 1-2, 1-224, 4-3, 4-6, 4-11, 4-17, 4-19, 4-48

### PREPARATION EXAMPLE 1

### Granule Formulation

To a mixture including the compound of the present invention (Compound No. 1-2; 10 parts by weight), bentonite (montmorilonite; 30 parts by weight), talc (58 parts by weight) and lignin sulfonate (2 parts by weight), water (25 parts by weight) is added and the resulting mixture is kneaded well. By using an extrusive granulator, granules of 10 to 40 mesh are formed and a granule formulation is obtained after drying at 40 to 50° C.

### PREPARATION EXAMPLE 2

### Granule Formulation

Clay mineral having a size distribution in the range of 0.2-2 mm (95 parts by weight) is added to a rotary mixer. By spraying the compound of the present invention (Compound No. 1-2; 5 parts by weight) together with a liquid diluent under rotation, the clay is moistened followed by drying at 40 to 50° C. to obtain a granule formulation.

### PREPARATION EXAMPLE 3

### Emulsion

By mixing the compound of the present invention (Compound No. 1-2; 30 parts by weight), xylene (55 parts by weight), polyoxyethylene alkylphenyl ether (8 parts by weight) and calcium alkylbenzene slufonate (7 parts by weight) with stirring, an emulsion is obtained.

### PREPARATION EXAMPLE 4

### Wettable Agent

By mixing the compound of the present invention (Compound No. 1-2; 15 parts by weight), a mixture including white carbon (fine powders of hydrous non-crystalline silicon oxide) and powder clay (1:5 mixture; 80 parts by weight), and a condensate of sodium alkylnaphthalene sulfonate formalin (3 parts by weight) and sodium alkylbenzene slufonate (2 parts by weight) with pulverization, a wettable agent is obtained.

### PREPARATION EXAMPLE 5

### Wettable Granules

The compound of the present invention (Compound No. 1-2; 20 parts by weight), lignin sodium sulfonate (30 parts by weight), bentonite (15 parts by weight) and calcined diatomaceous earth powder (35 parts by weight) are thoroughly mixed. After adding water thereto, the mixture is extruded through 0.3 mm screen followed by drying to obtain wettable granules.

The novel pesticidal condensed-ring aryl derivatives of the present invention have an excellent pesticidal activity as shown in the biological examples.

1. A compound of formula (I) 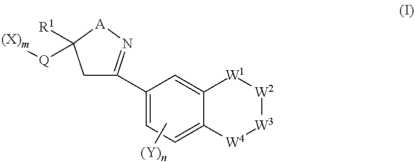
wherein
X is halogen, nitro, cyano, hydroxy, thio, amino, C₁₋₁₂ alkyl, C₁₋₁₂ haloalkyl, C₁₋₁₂ alkoxy, C₁₋₁₂ haloalkoxy, C₁₋₁₂ alkylsulfenyl, C₁₋₁₂ alkylsulfinyl, C₁₋₁₂ alkylsulfonyl, C₁₋₁₂haloalkyl-sulfenyl, C₁₋₁₂ haloalkylsulfinyl, C₁₋₁₂ haloalkylsulfonyl, C₁₋₁₂ alkylamino, C₂₋₂₄ dialkylamino, C₁₋₁₂ acylamino, C₁₋₁₂ alkoxy-carbonylamino, C₁₋₁₂ haloalkoxy-carbonylamino, C₁₋₁₂ alkylsulfonylamino, or C₁₋₁₂haloalkylsulfonylamino;
Q is optionally substituted phenyl, naphthyl, or a optionally substituted 5- or 6-membered heterocyclic group;
Y is halogen, nitro, cyano, hydroxy, thiol, amino, C₁₋₁₂ alkyl, C₁₋₁₂ haloalkyl, C₃₋₈ cycloalkyl, C₃₋₈ cyclo-haloalkyl, C₂₋₁₂ alkenyl, C₂₋₁₂ haloalkenyl, C₁₋₁₂ alkoxy, C₁₋₁₂ haloalkoxy, C₁₋₁₂ alkylsulfenyl, C₁₋₁₂ alkylsulfinyl, C₁₋₁₂ alkylsulfonyl, C₁₋₁₂ haloalkylsulfenyl, C₁₋₁₂ haloalkylsulfonyl, C₁₋₁₂ haloalkylsulfonyl, C₁₋₁₂ alkylamino, C₂₋₂₄ dialkylamino, C₁₋₁₂ aminocarbonyl, C₁₋₁₂ alkylamino-carbonyl, C₂₋₂₄ dialkylamino-carbonyl, C₁₋₄₂ acylamino, C₁₋₁₂ alkoxy-carbonyl-amino, benzyloxy-carbonylamino, C₁₋₁₂ haloalkoxy-carbonylamino, C₁₋₁₂ alkyl-sulfonylamino, C₁₋₁₂ haloalkylsulfonylamino, or C₃₋₃₆ trialkylsilyl;
R¹ is cyano, C₁₋₁₂ alkyl, C₃₋₈ cycloalkyl, C₄₋₂₀ alkyl-cycloalkyl, C₄₋₂₀ cycloalkylalkyl, C₂₋₁₂ alkenyl, C₂₋₁₂ alkynyl, C₁₋₁₂ haloalkyl, or C₃₋₈ halocycloalkyl;
m is 0, 1, 2, 3, 4, or 5;
n is 0, 1, 2, or 3;
A is O, S, CH₂, or N—R²;
R² is hydrogen, cyano, formyl, C₁₋₁₂ alkyl, C₂₋₁₂ alkenyl, C₂₋₁₂ alkynyl, C₃₋₈ cycloalkyl, C₄₋₂₀ alkylcycloalkyl, C₄₋₂₀ cyclo-alkylalkyl, C₁₋₁₂ haloalkyl, C₁₋₁₂ alkylsulfonyl, C₁₋₁₂ haloalkylsulfonyl, phenyl, C₁₋₁₂ alkyl-carbonyl, C₁₋₁₂ alkoxy-carbonyl, C₁₋₁₂ alkylamino-carbonyl, or C₂₋₂₄ dialkylamino-carbonyl;
W¹, W², W³, and W⁴ each independently is a single bond, CH₂, CH, N, —N⁺(O⁻)—, —S(O)—, —S(O)₂—, —O—S(O)—, O, S, C(R³)—R³, C—R³, C—R⁴, C(R³)—R⁴, C(R⁴)—R⁴, C—N(R³)—R³, C(R³)—N(R³)—N(R³)—R³, C—N(R³)—N(R³)—R³, C(R³)—N(R⁴)—N(R³)—R³, C—N(R⁴)—N(R³)—R³, C(R³)—N(R³)—OR³, C—N(R³)—OR³, C(R³)—OR³, C—OR³, C(R³)—SR³, C—SR³, C—N₃, N—R³, N—OR³, N—N(R³)—R³, N—R⁴, or C═U with the proviso that
(i) not more than two of W¹, W², W³, and W⁴ are a single bond; and/or
(ii) not more than two of W¹, W², W³, and W⁴ are O, S, N—R³, N—R⁴, C—N(R³)—R³, C—N(R³)—N(R³)—R³, C—N(R⁴)—N(R³)—R³, C(R³)—N(R³)—OR³, C—N(R³)—OR³, C—SR³, N—R³, N—OR³, or N—N(R³)—R³; and/or
(iii) not more than two of W¹, W², W³, and W⁴ are C═U; and/or
(iv) if two of W¹, W², W³, and W⁴ are O or S then at least one carbon atom is present between them; and/or
(v) when one of W¹, W², W³, and W⁴ is CH, N, C—R³, C—R⁴, C—N(R³)—R³, C—N(R³)—N(R³)—R³, C—N(R⁴)—N(R³)—R³, C—N(R³)—OR³, C—OR³, C—SR³, N—R³, N—OR³, or N—N(R³)—R³, a double bond is formed within the condensed ring;
U is CH₂, O, S, or N—R³, or N—R⁴;
R³ each independently is hydrogen, hydroxy, thiol, amino, cyano, formyl, halogen, nitro C₁₋₆ alkyl, C₂₋₁₂ alkoxyalkyl, C₂₋₁₂ haloalkoxyalkyl, C₂₋₆ alkenyl, C₂₋₁₂ alkynyl, C₃₋₈ cycloalkyl, C₄₋₁₂ alkylcycloalkyl, C₄₋₁₂ cycloalkylalkyl, C₁₋₆haloalkyl, C₁₋₆ alkylcarbonyl, C₁₋₆ alkylcarbonyl-C₁₋₆ alkyl, C₁₋₆ alkyl-carbonyl-C₁₋₆ alkylcarbonyl, C₁₋₆ haloalkylcarbonyl, C₁₋₆ alkoxycarbonyl, C₁₋₆ alkylsulfenylcarbonyl, C₁₋₆haloalkylsulfenylcarbonyl, aminocarbonyl, C₁₋₆ alkylaminocarbonyl, C₁₋₆haloalkylaminocarbonyl, C₁₋₆ hydroxyalkylamino-carbonyl, C₂₋₁₂ dialkylamino-carbonyl, C₂₋₆ di(haloalkyl)aminocarbonyl, C₂₋₆ alkenylaminocarbonyl, C₂₋₆ alkynylaminocarbonyl, C₁₋₆alkyl-thiocarbonyl, C₃₋₆ cycloalkylcarbonyl, C₄₋₁₂ cycloalkylalkyl-carbonyl, C₃₋₆ cycloalkyl-thiocarbonyl, C₄₋₁₂ cycloalkylalkyl-thiocarbonyl, C₁₋₆haloalkyl-thiocarbonyl, C₁₋₆ alkylamino-thiocarbonyl, C₃₋₆ cycloalkylamino-carbonyl, C₄₋₁₂ cycloalkyl-alkylamino-carbonyl, C₃₋₆ cycloalkylamino-thiocarbonyl, C₄₋₁₂ cycloalkylalkyl-aminothiocarbonyl, C₁₋₆haloalkylamino-thiocarbonyl, C₂₋₁₂ dialkylamino-thiocarbonyl, C₃₋₆ cycloalkyloxy-carbonyl, C₄₋₁₂ cycloalkylalkyloxy-carbonyl, C₁₋₆ haloalkoxy-carbonyl, C₁₋₆ alkylsulfonyl, C₁₋₆haloalkylsulfonyl, phenyl-sulfonyl, R⁴—C₁₋₆ alkyl, R⁴-carbonyl; 4-thiocarbonyl, R⁴—C₁₋₆ alkylcarbonyl, R⁴—C₁₋₆ alkyl-thiocarbonyl, R⁴-oxycarbonyl, R⁴—C₁₋₆ alkyloxy-carbonyl, R⁴-amino-carbonyl, R⁴-amino-thiocarbonyl, R⁴—C₁₋₆ alkylamino-carbonyl, or R⁴—C₁₋₆ alkyl-amino-thiocarbonyl; and
R⁴ represents is phenyl or a 5- or 6-membered saturated or unsaturated heterocyclic ring.

2. The compound according to claim 1, wherein
Q is selected from optionally substituted Q-1 to Q-54 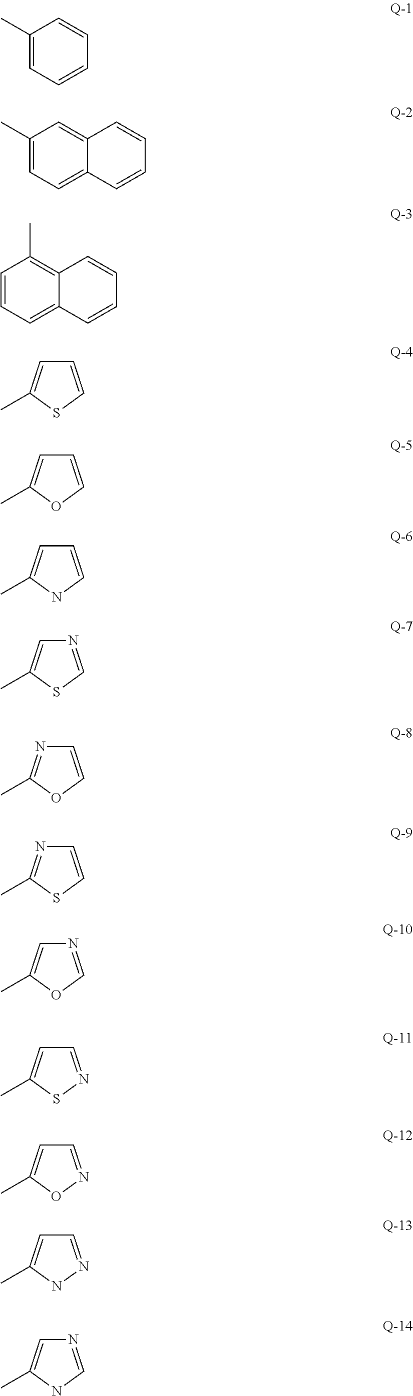 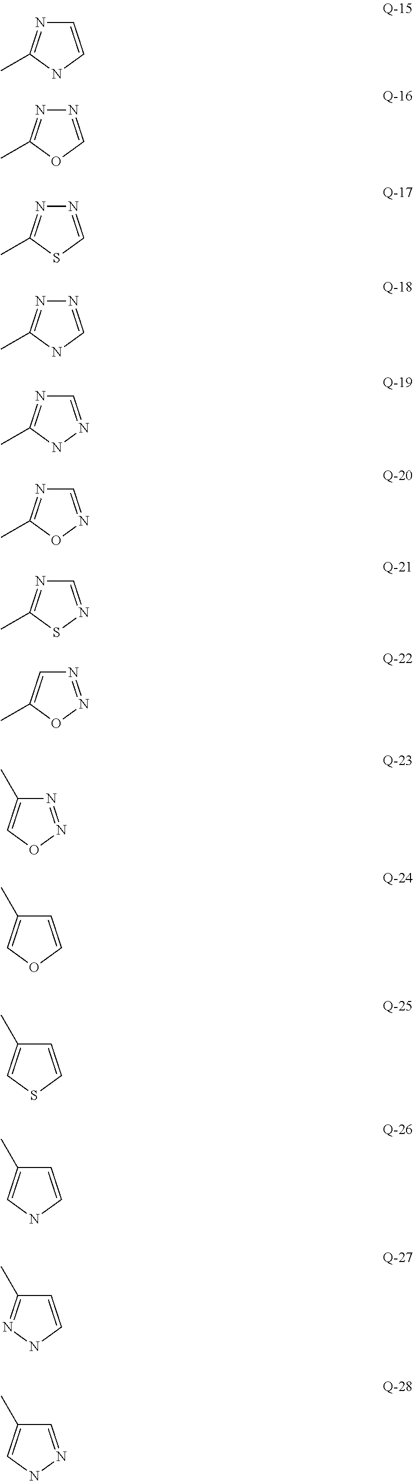 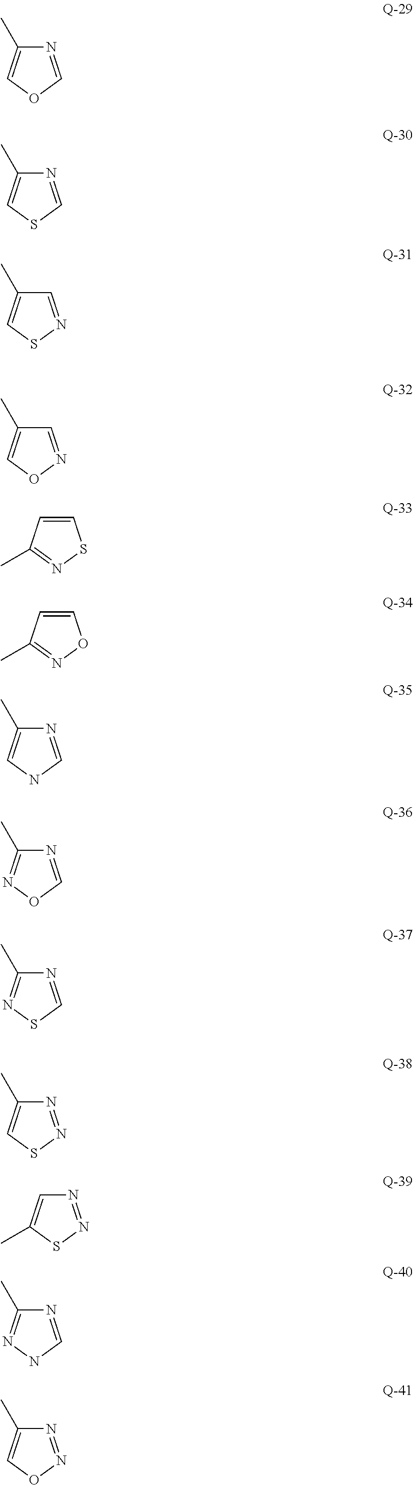 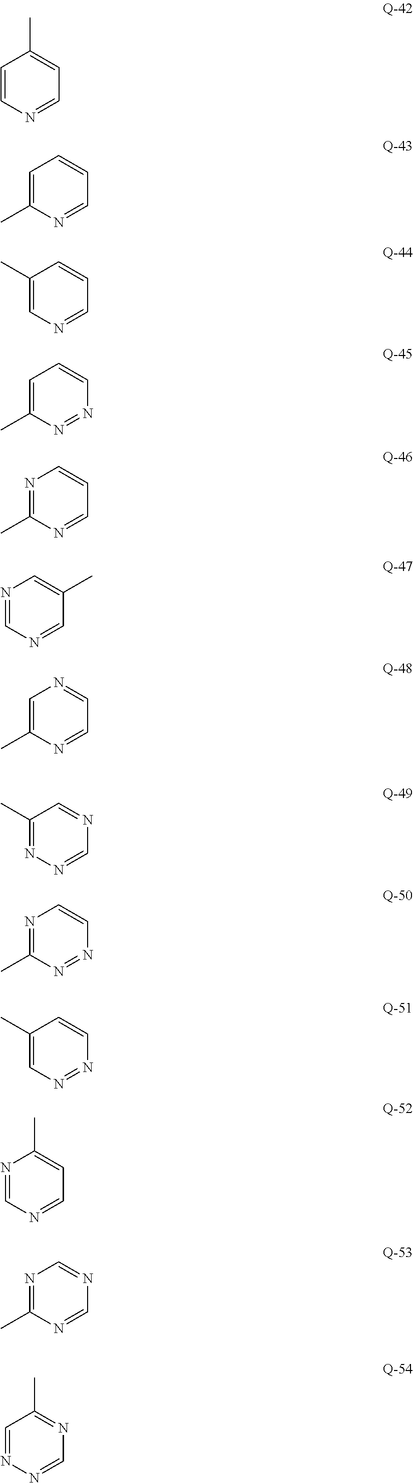
and
the grouping —W¹—W²—W³—W⁴—is selected from W-1 to W-580 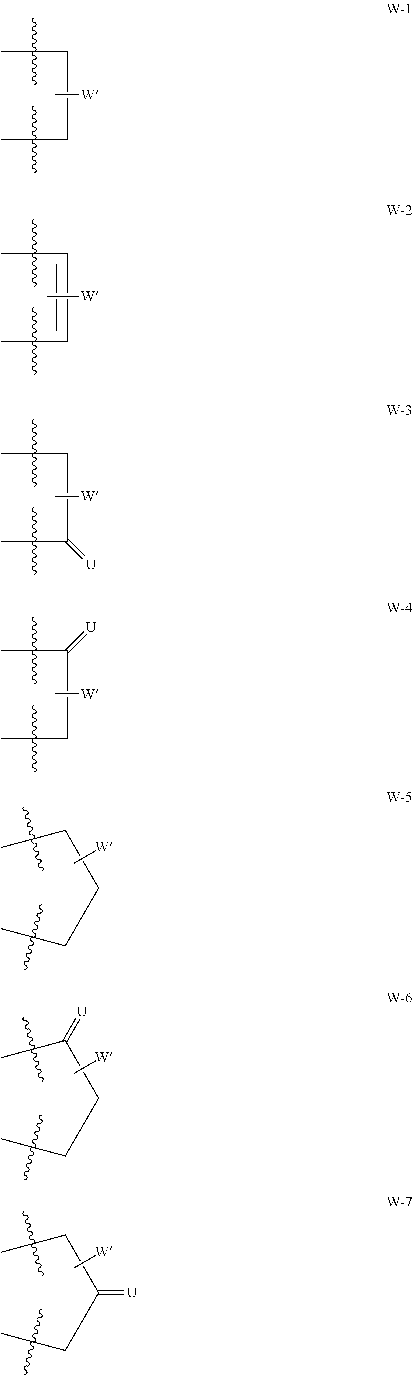 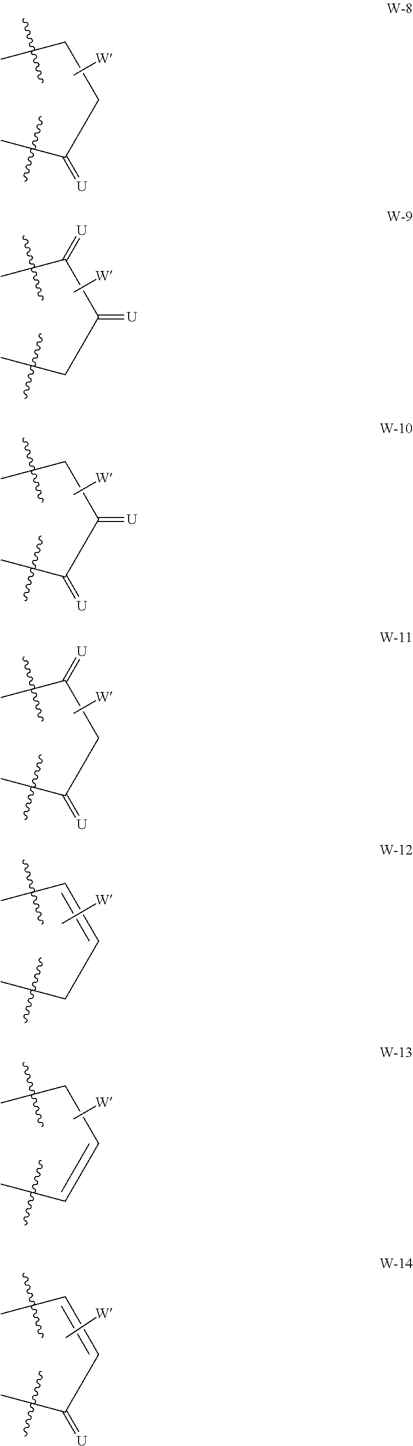 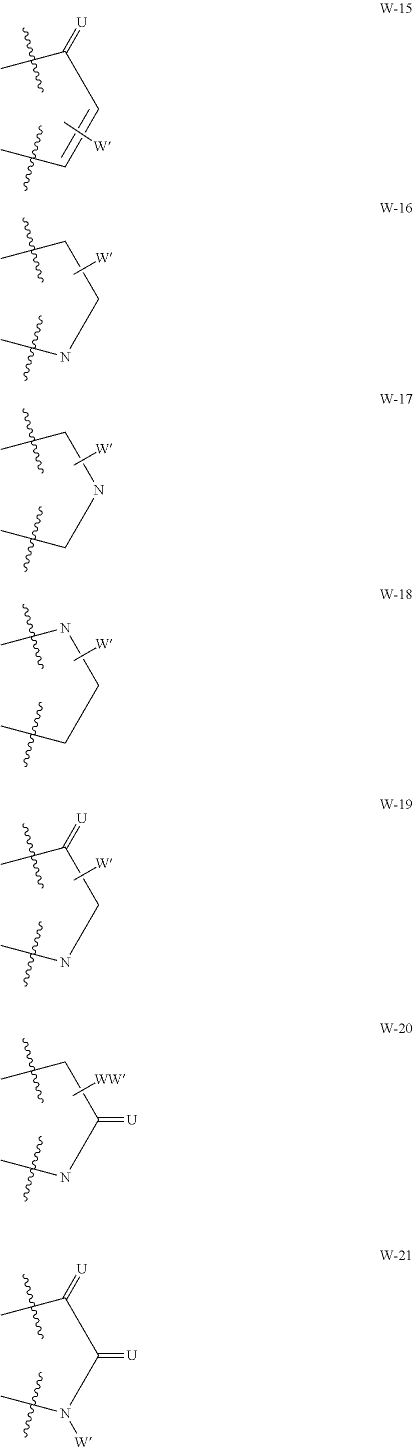 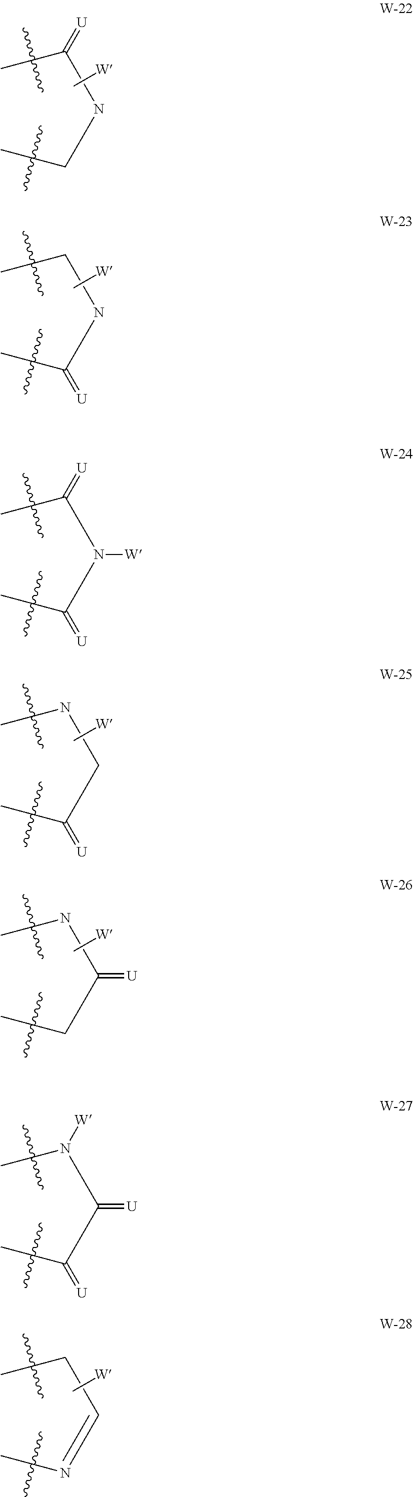 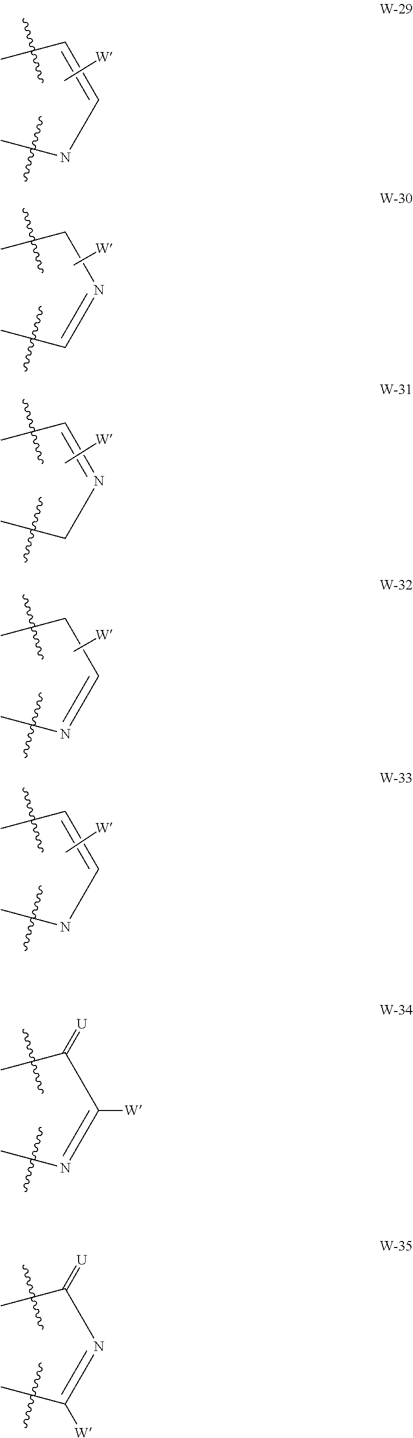 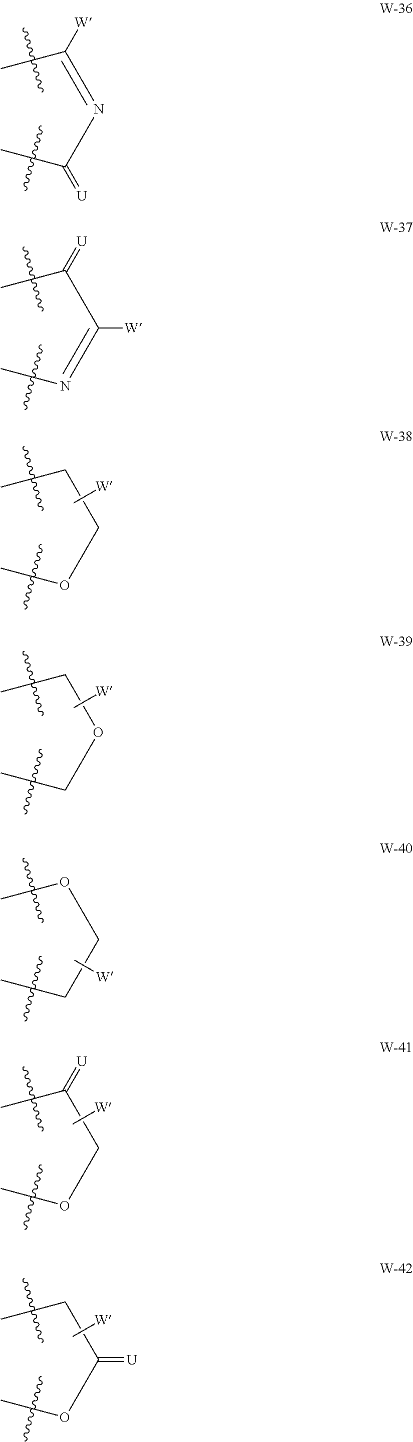 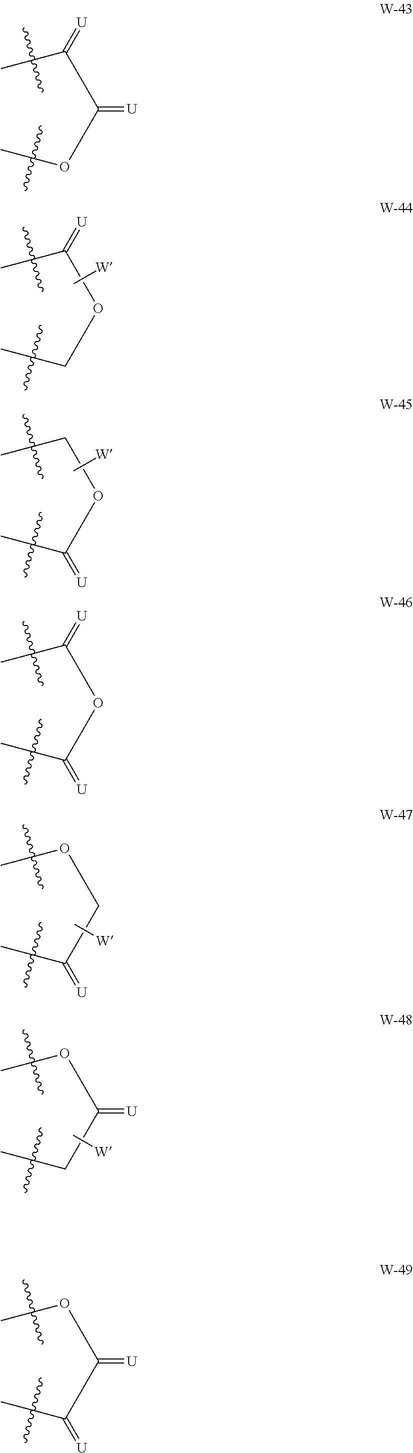 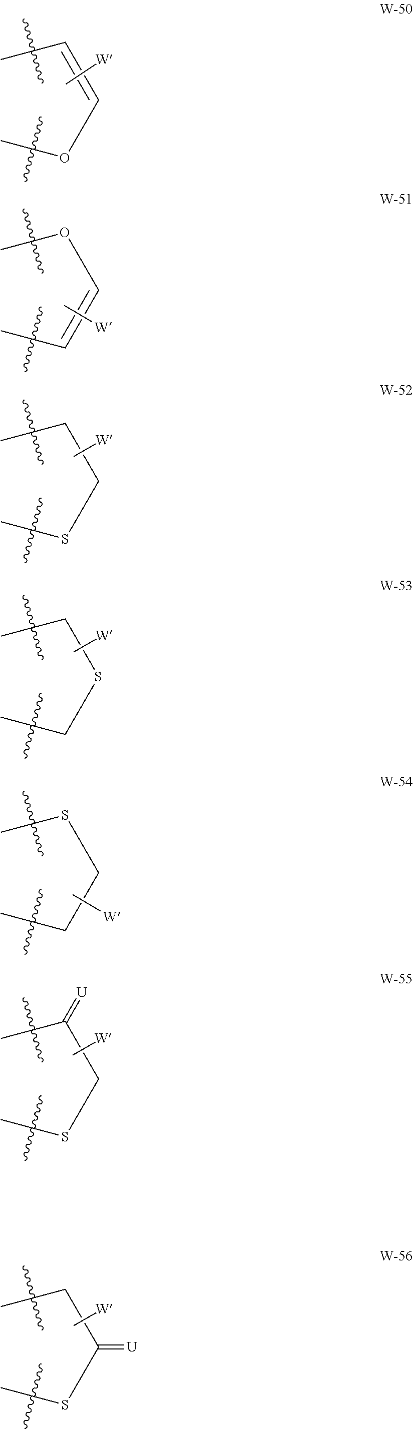 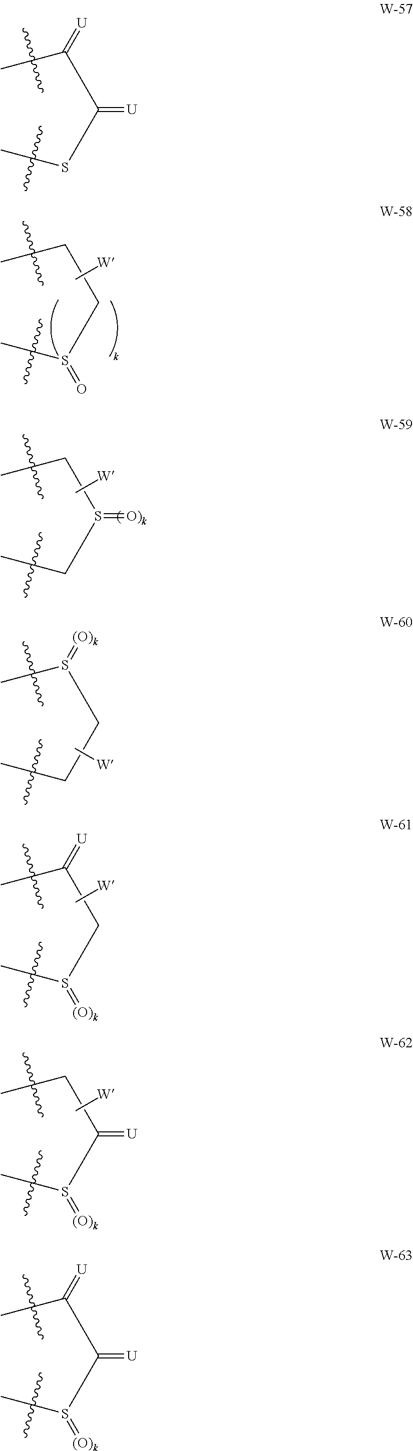 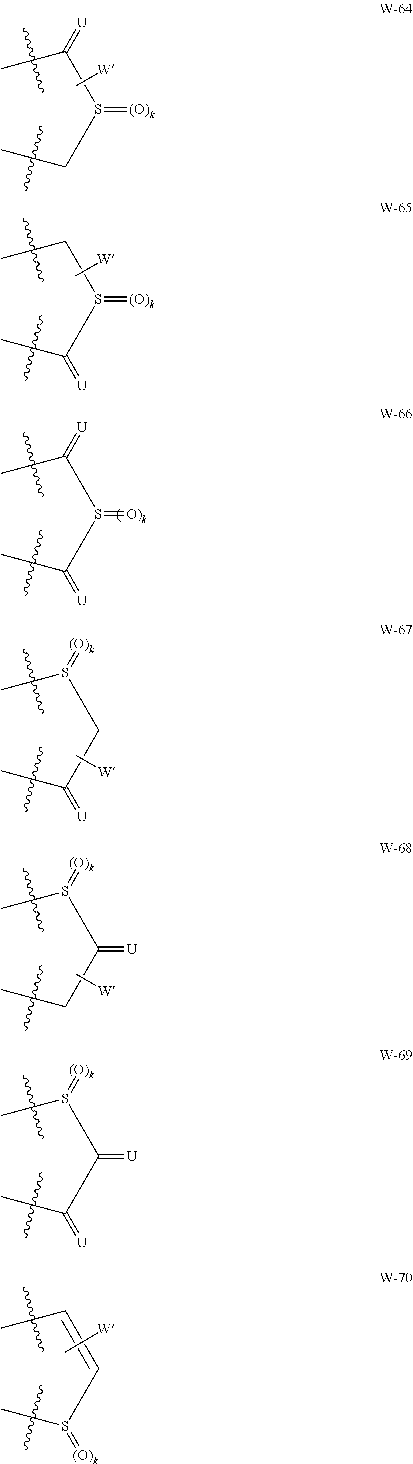 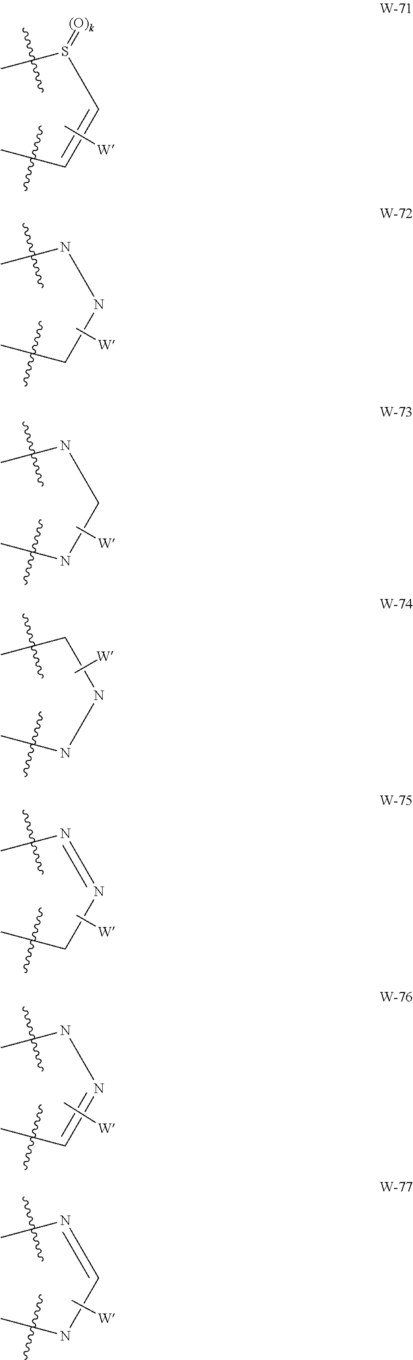 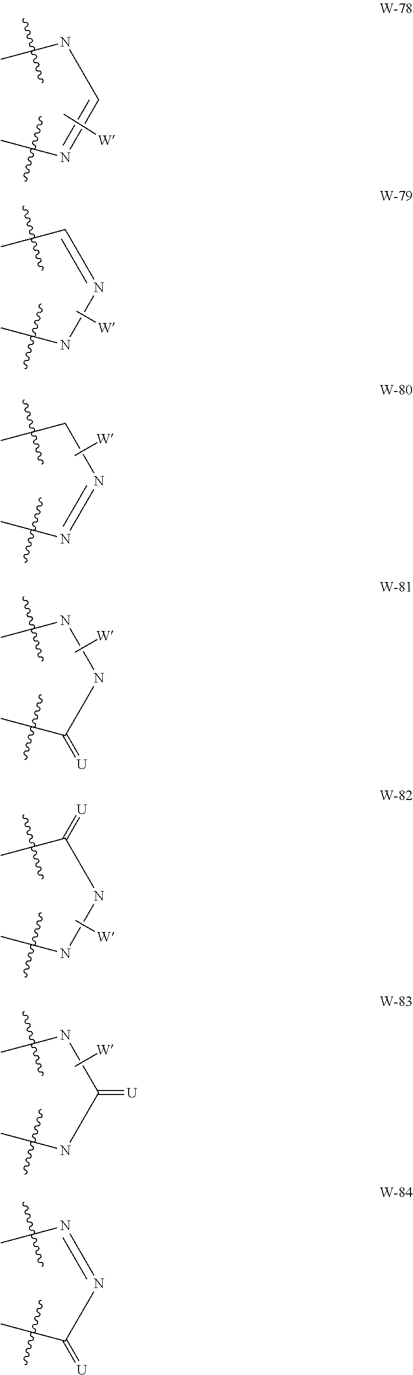 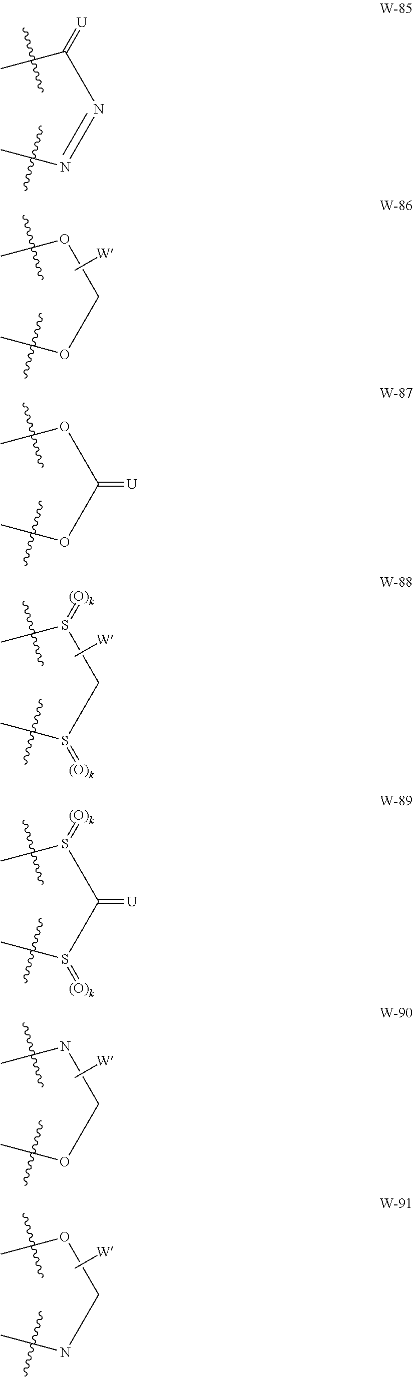 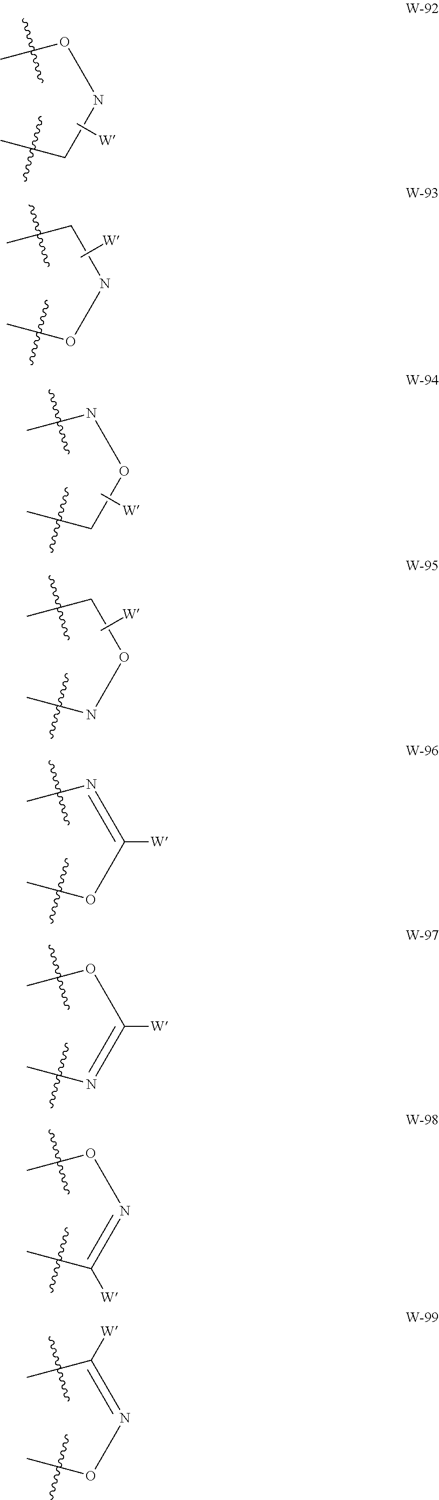 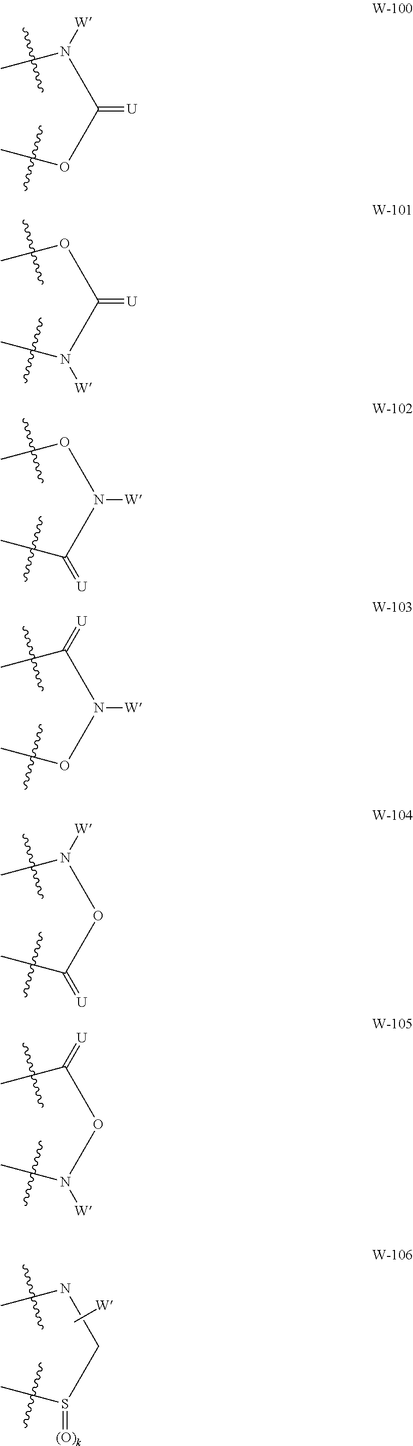 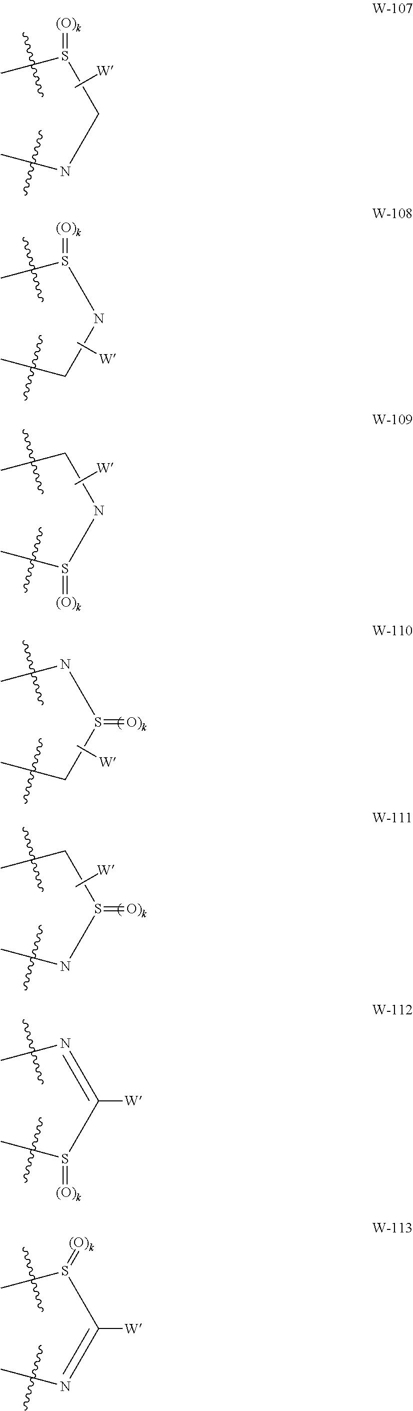 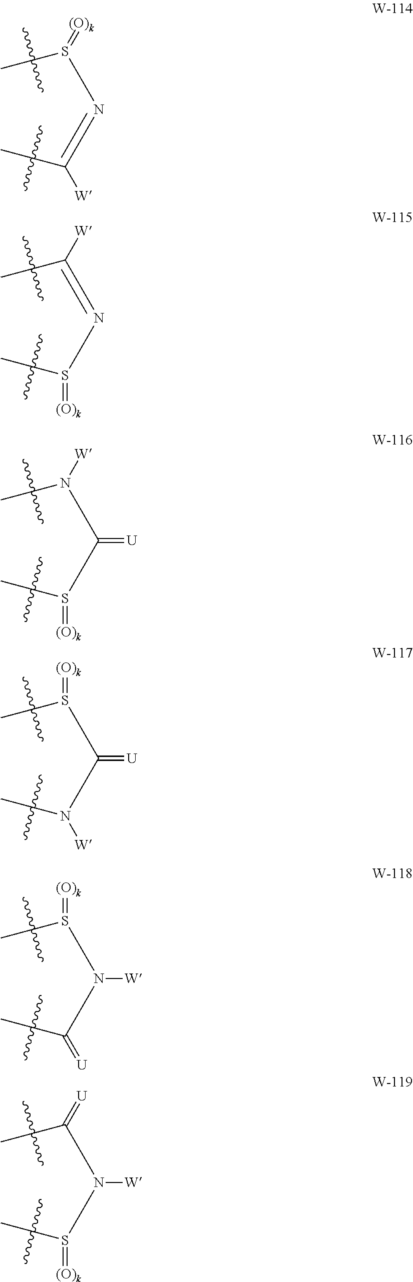 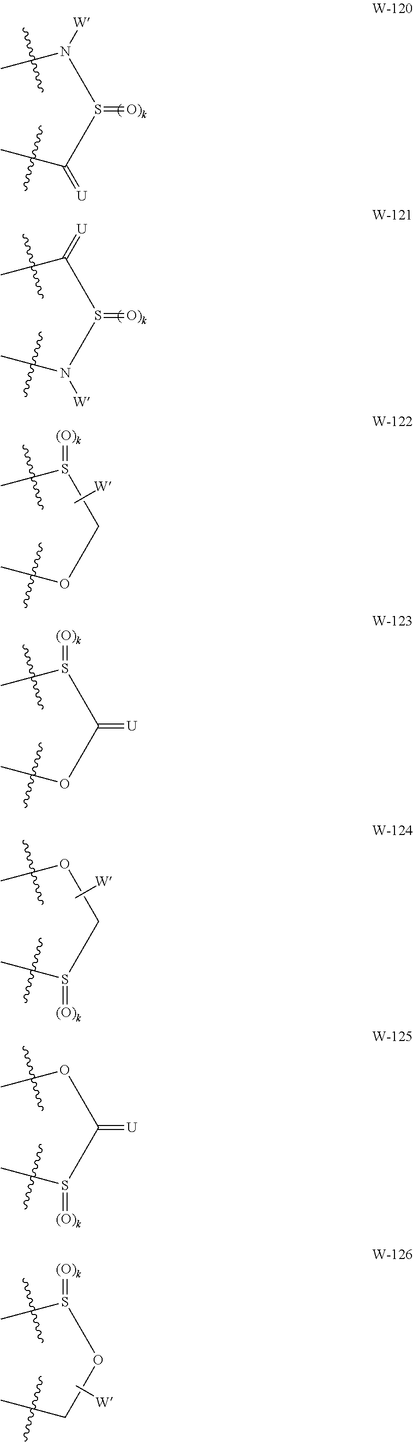 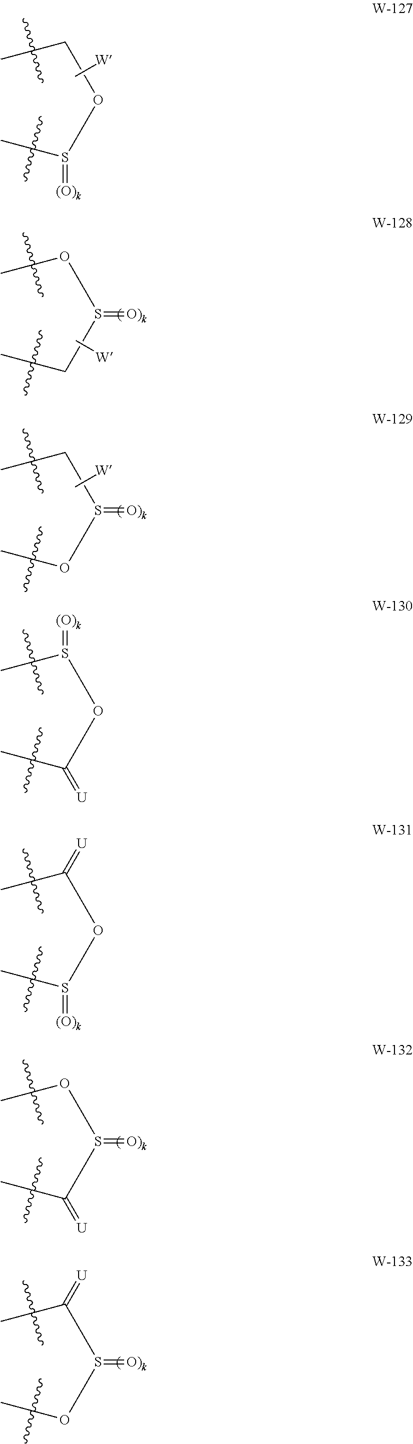 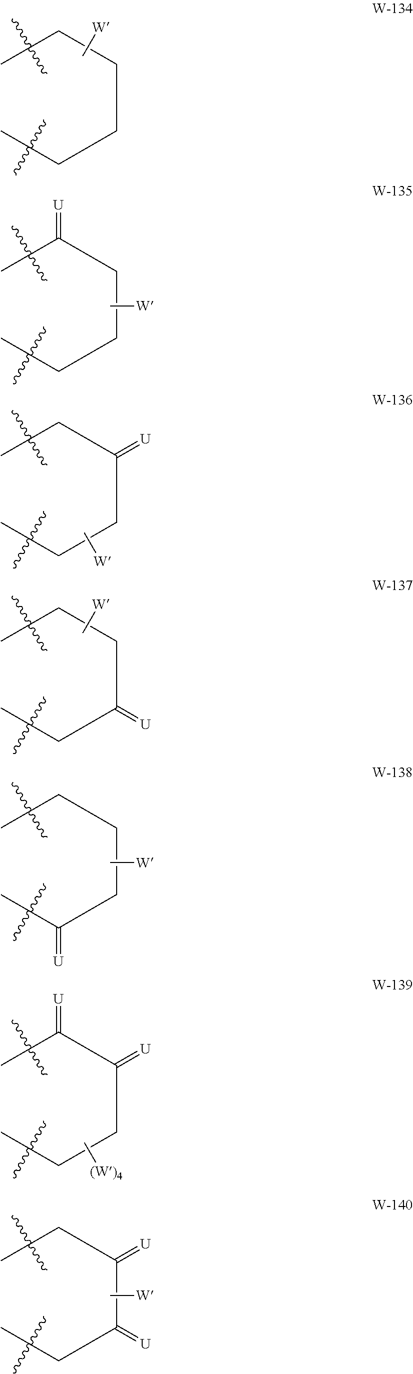 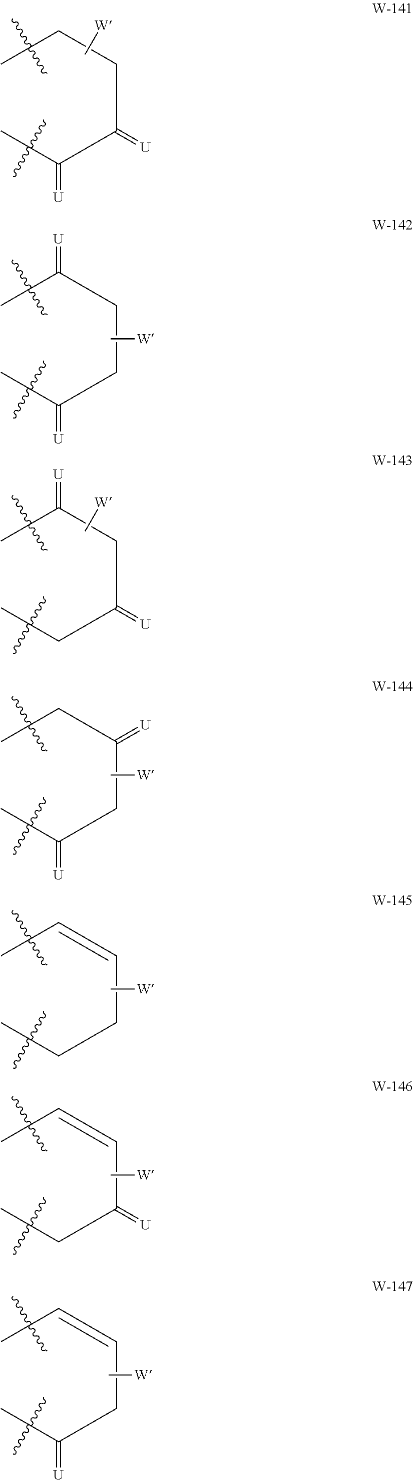 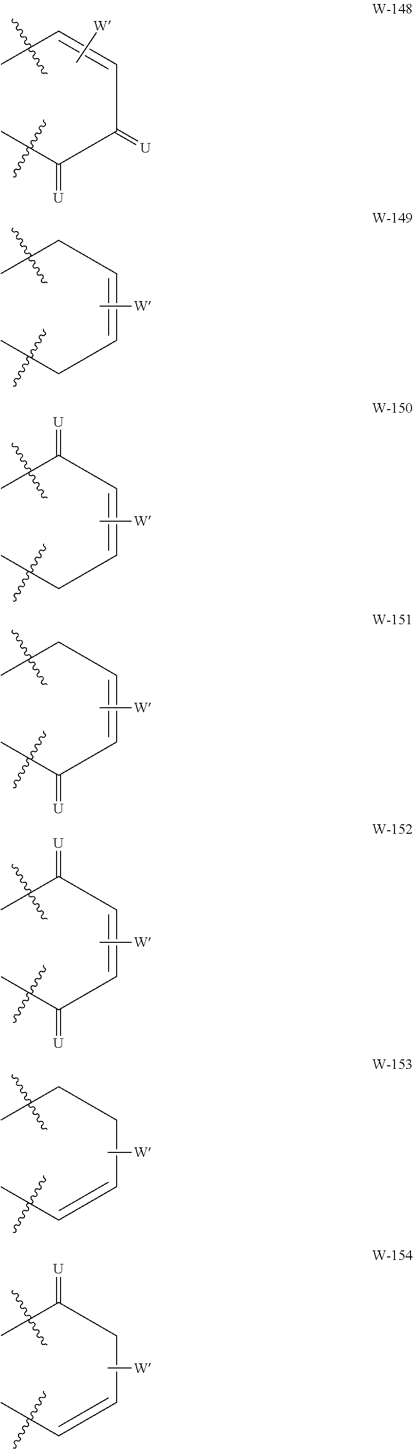 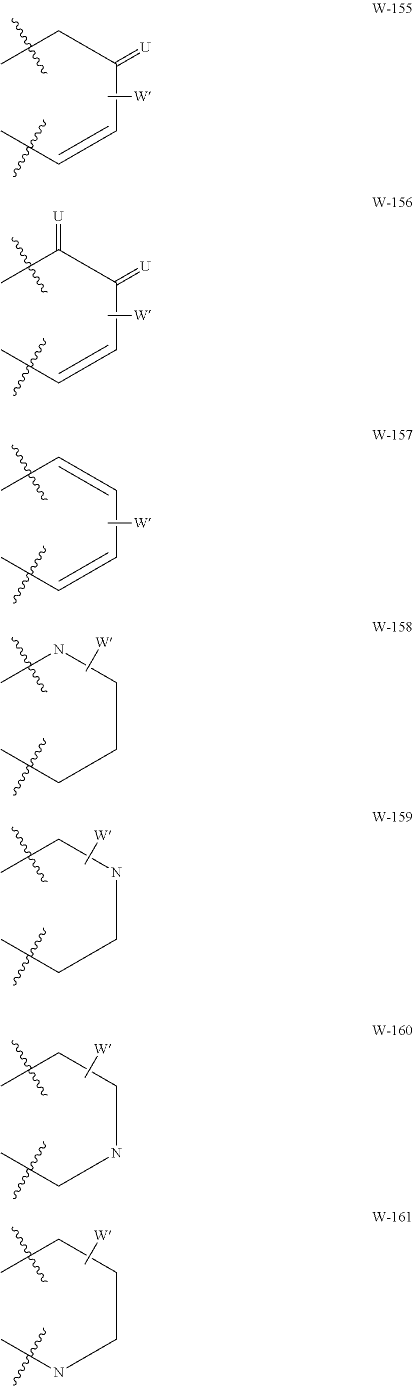 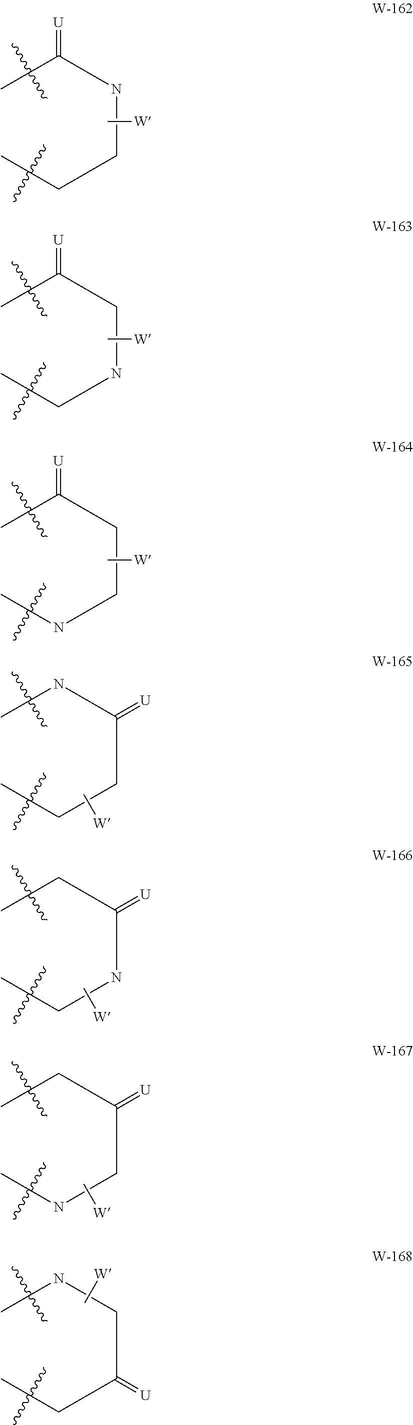 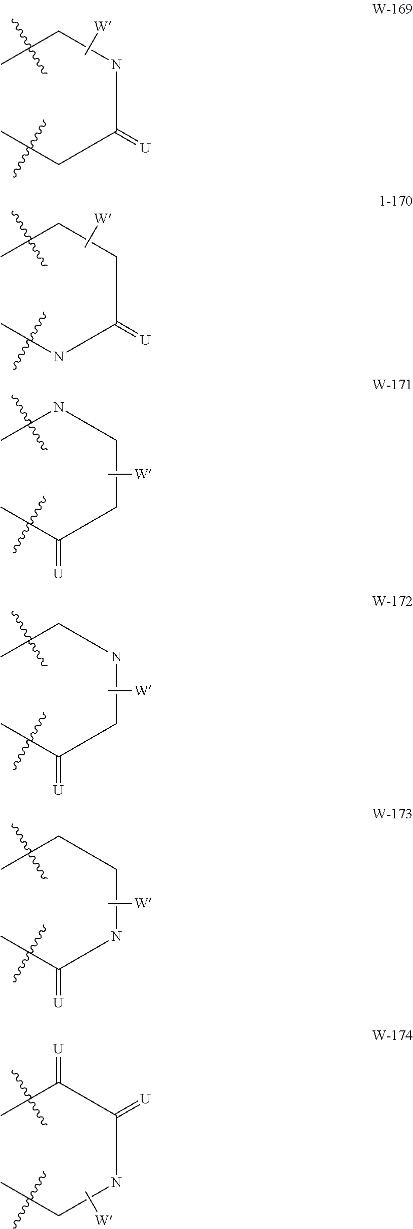 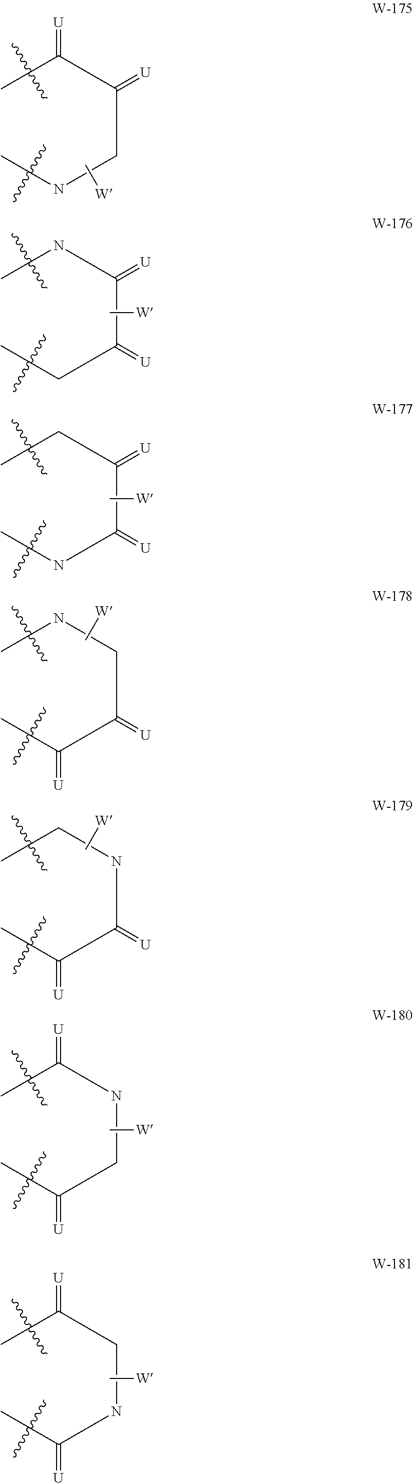 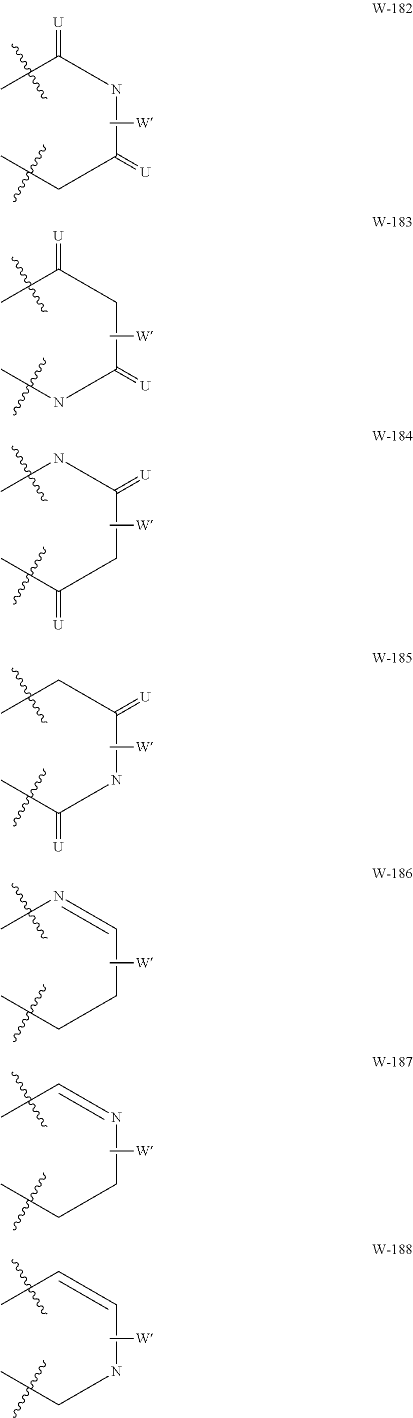 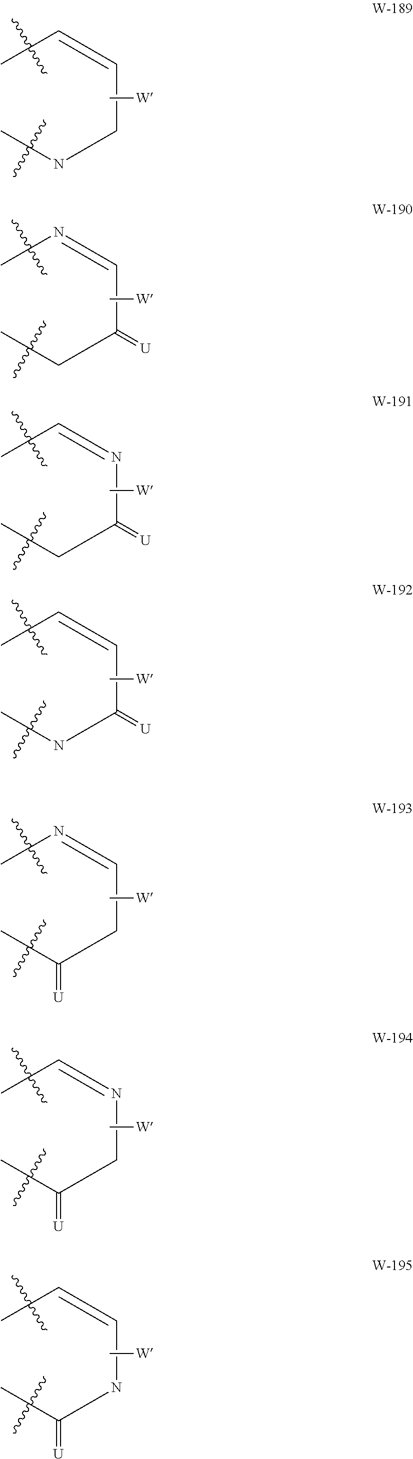 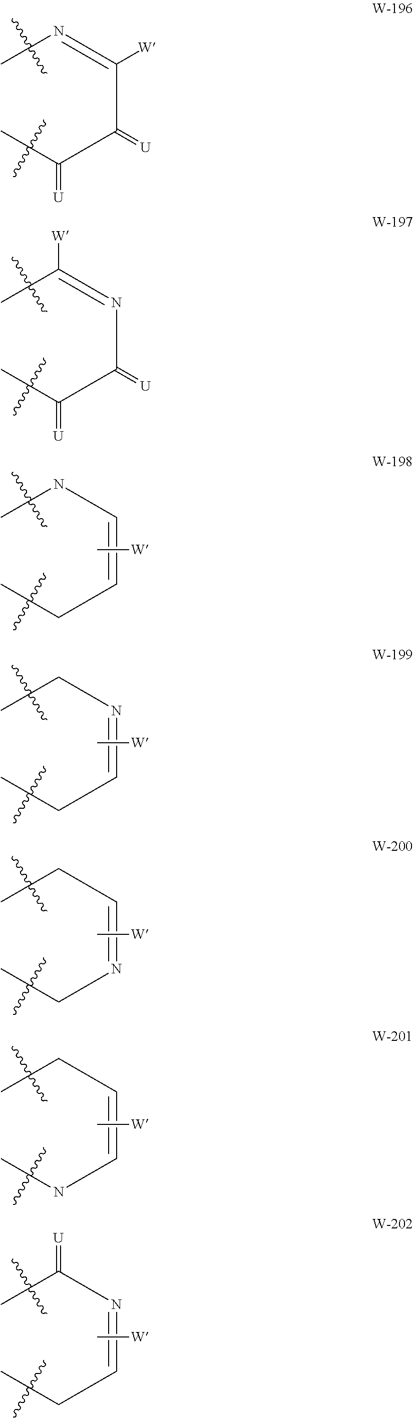 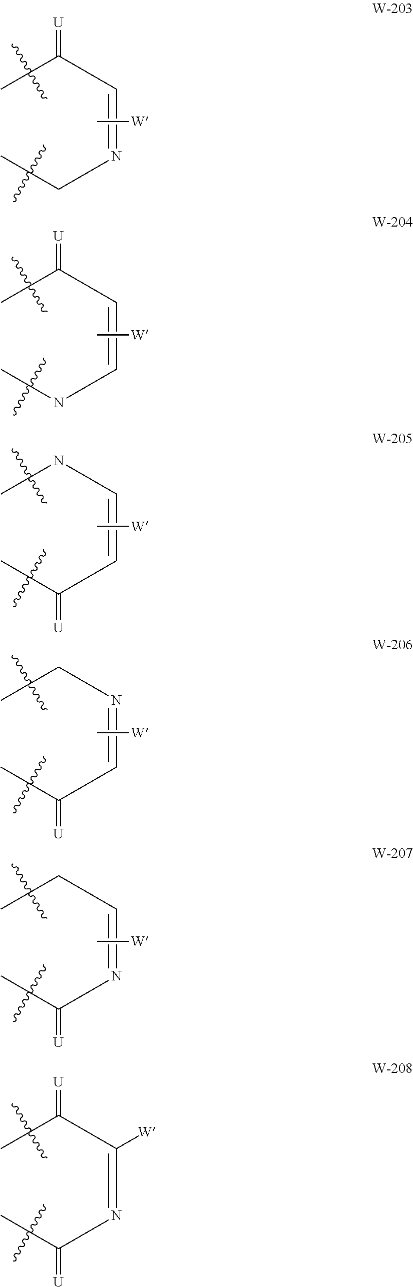 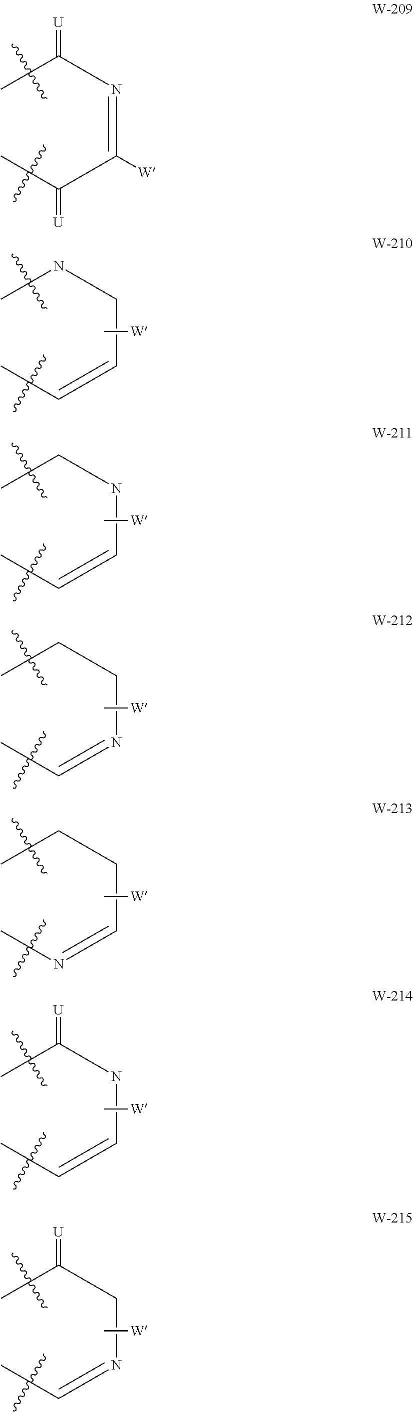 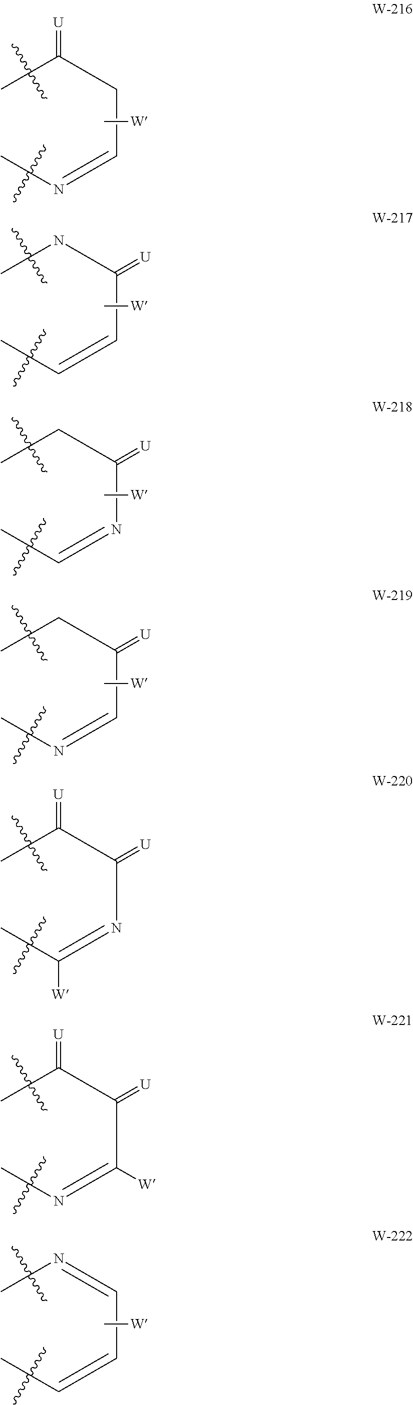 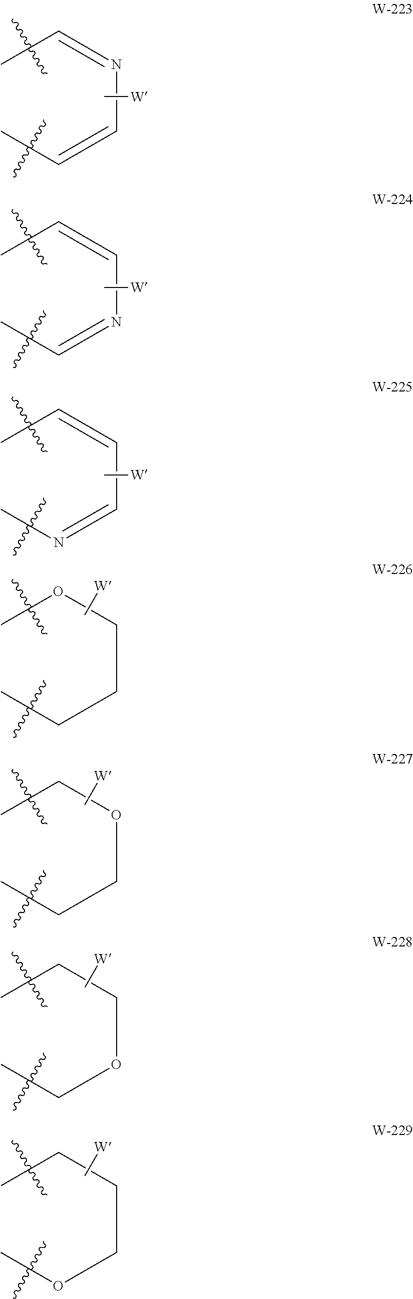 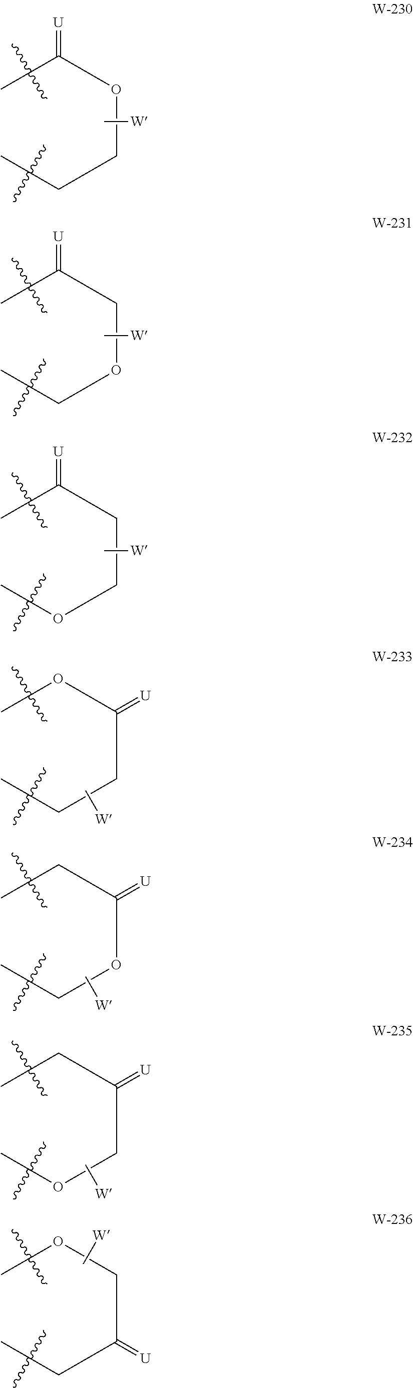 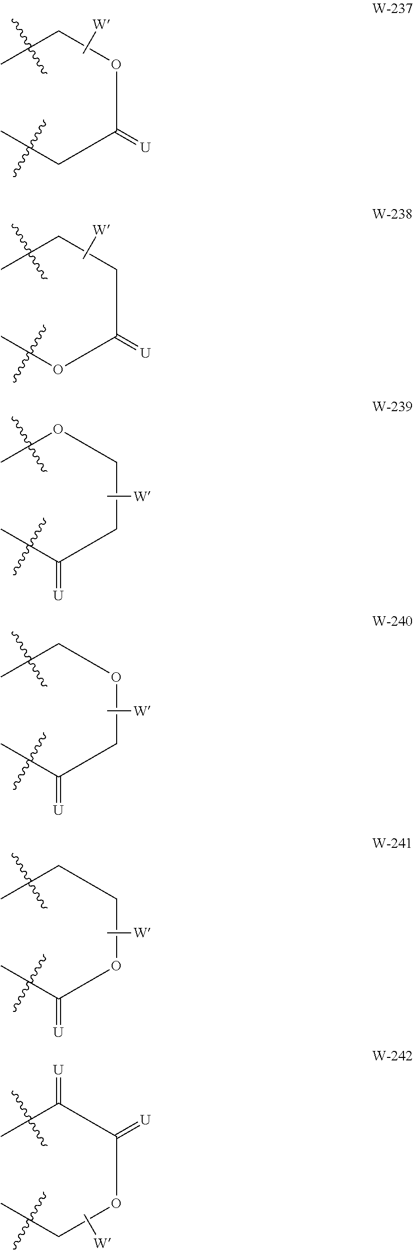 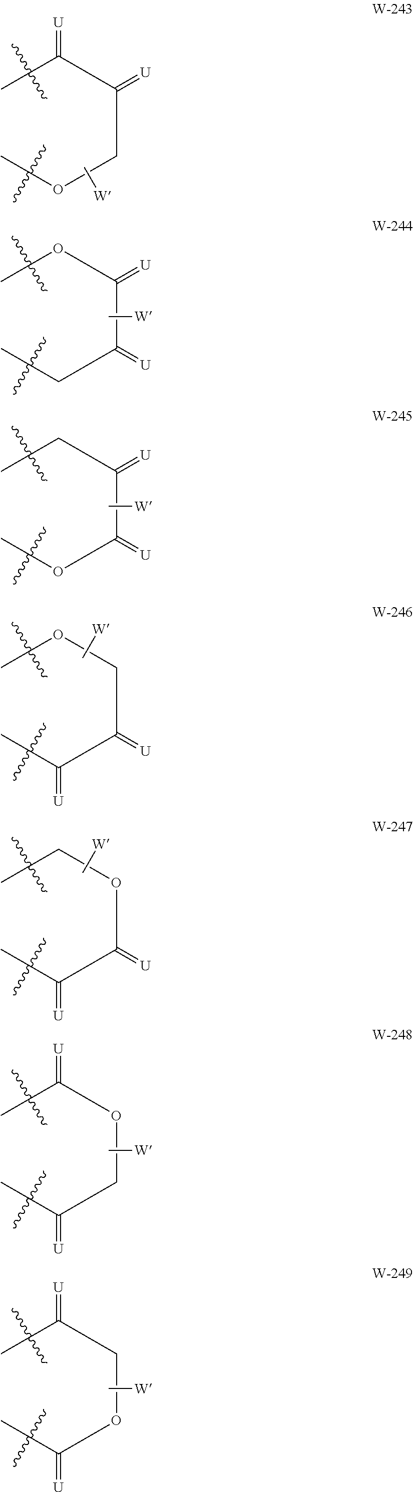 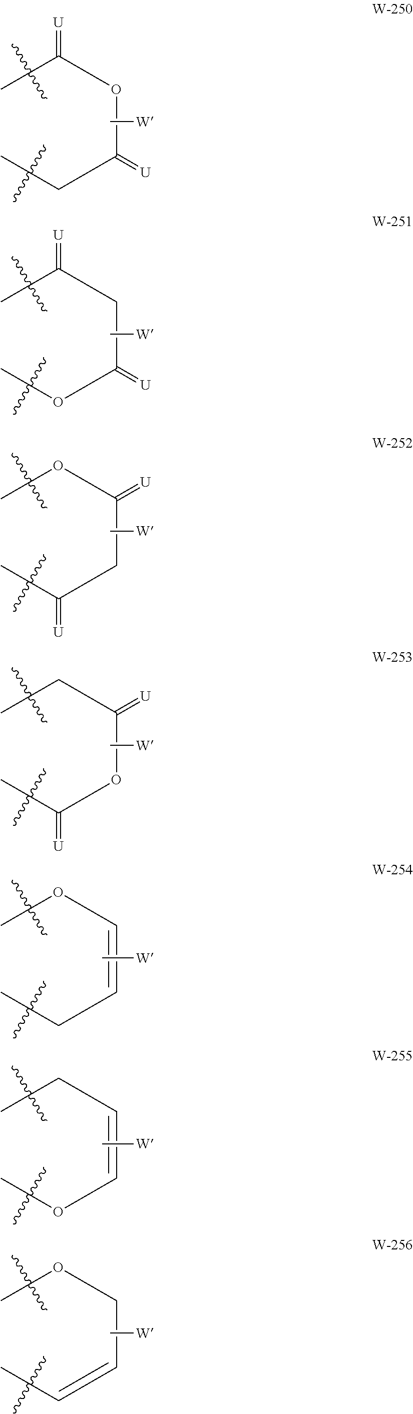 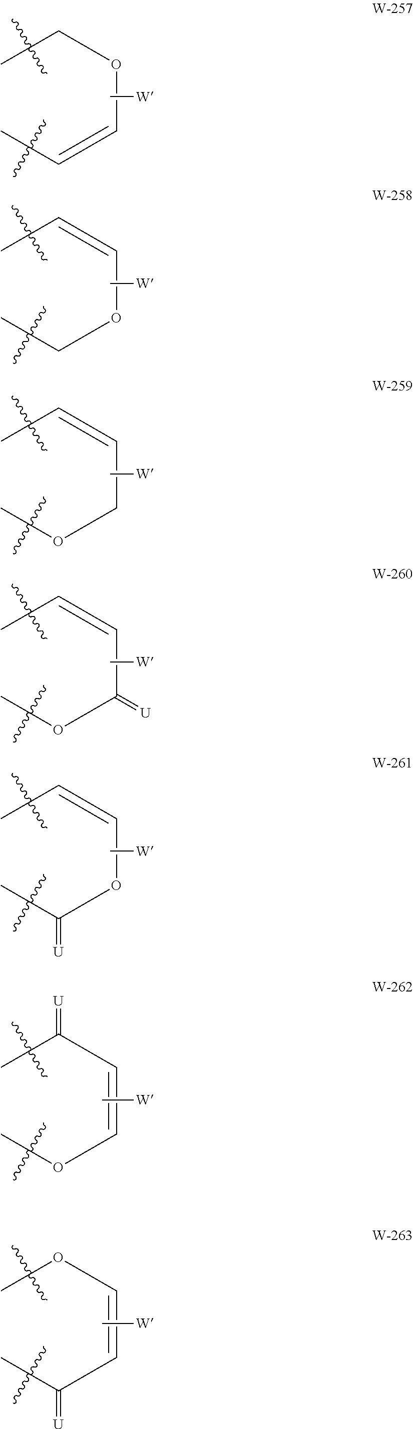 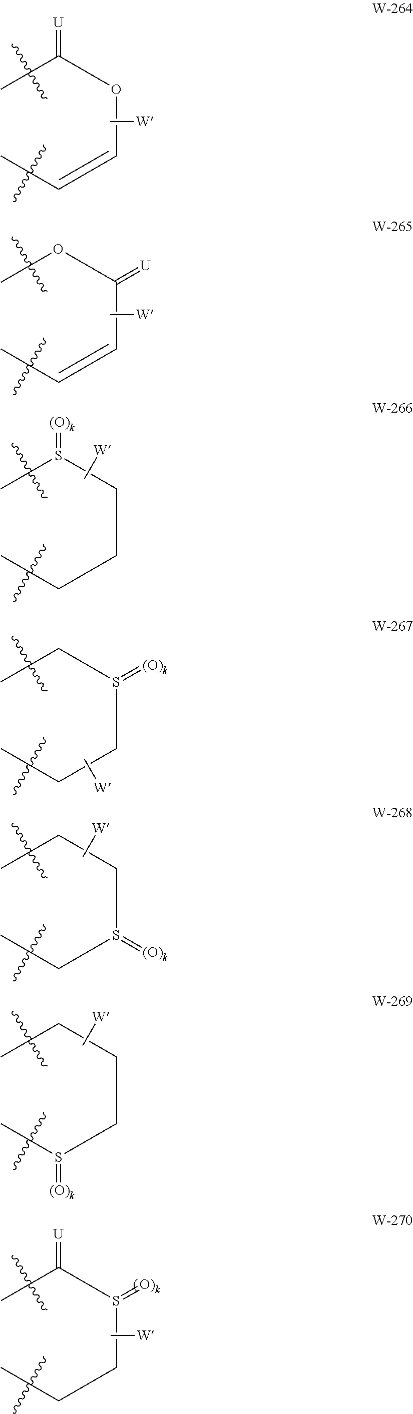 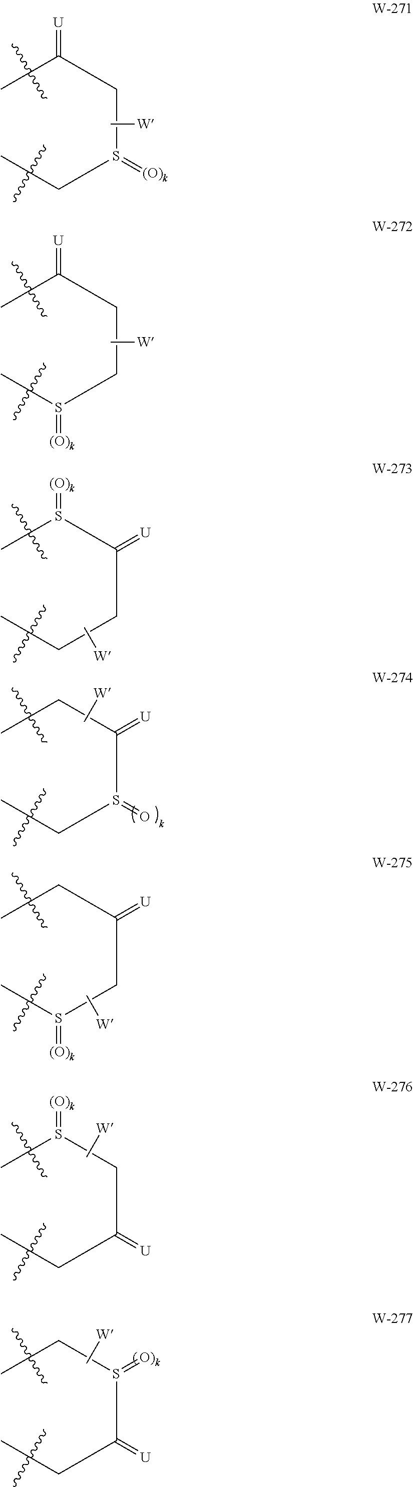 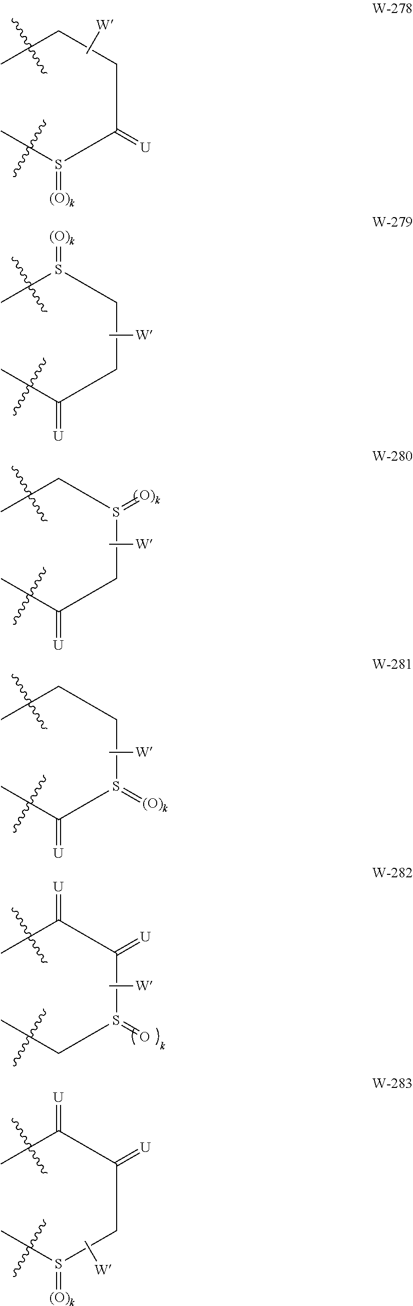 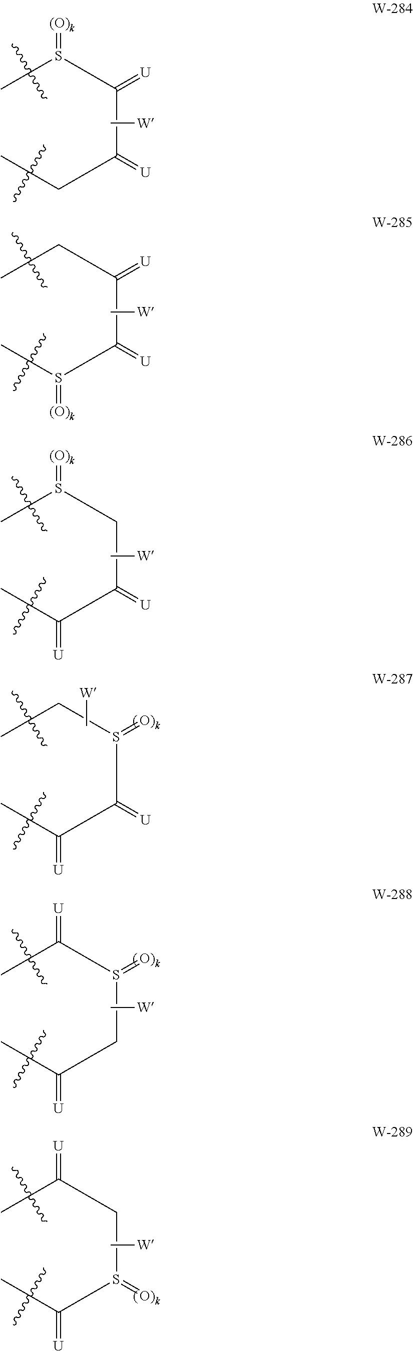 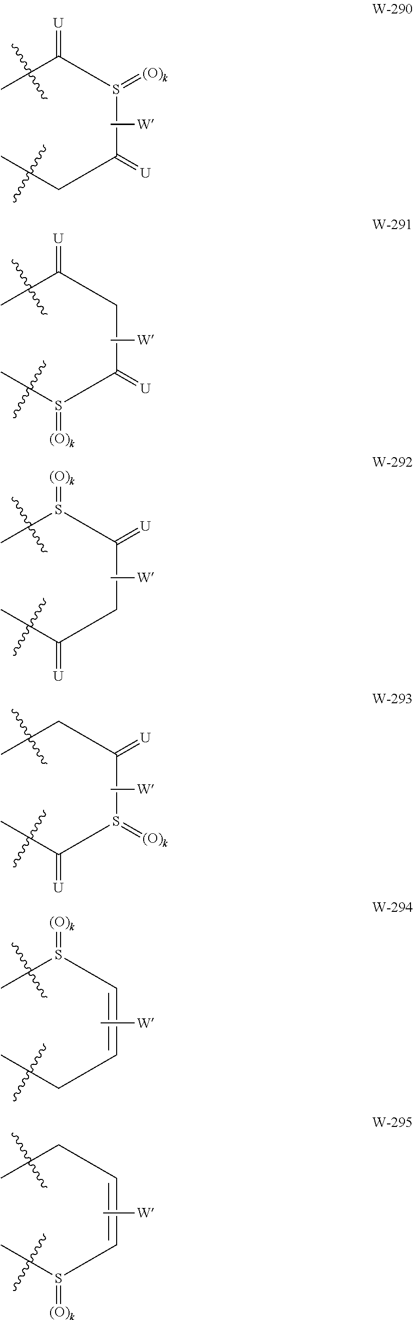 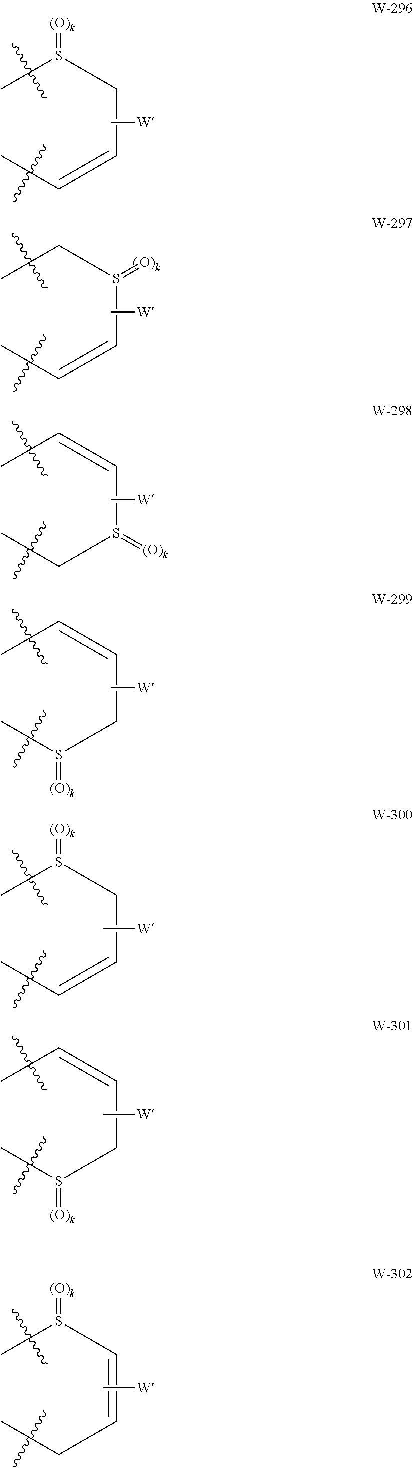 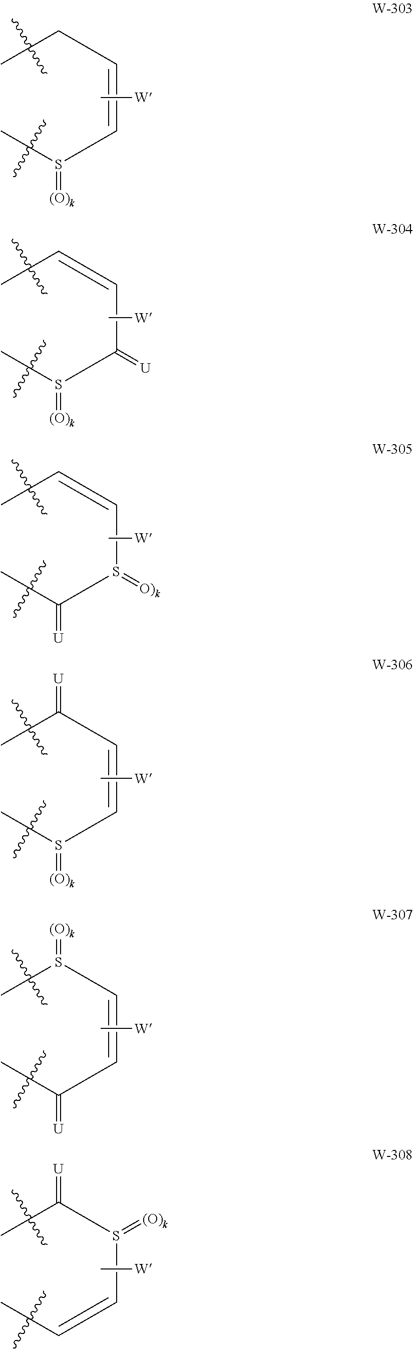 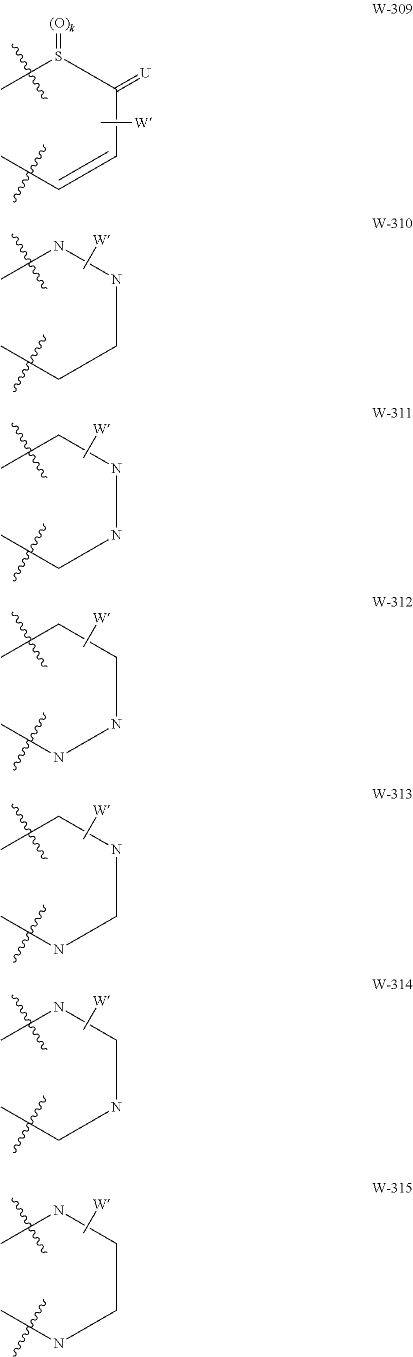 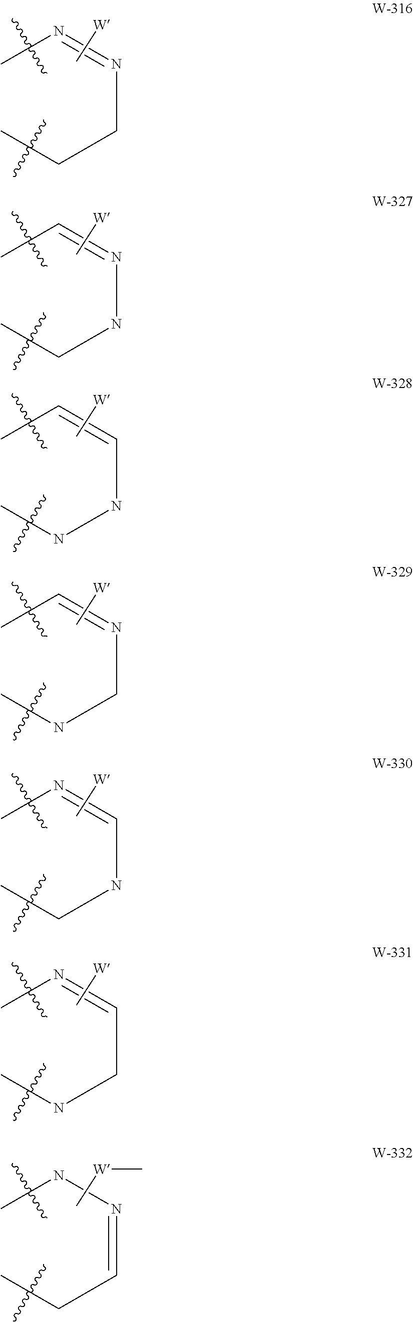 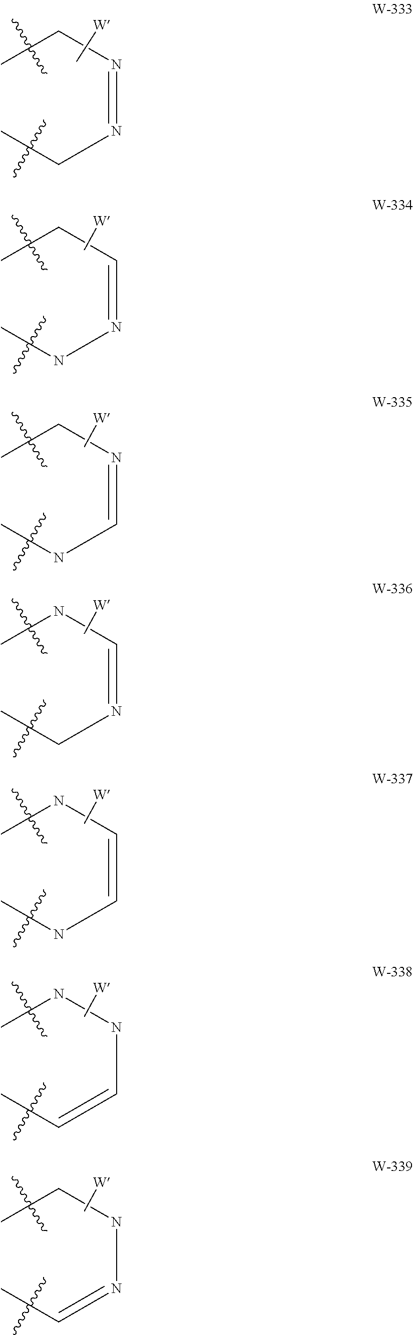 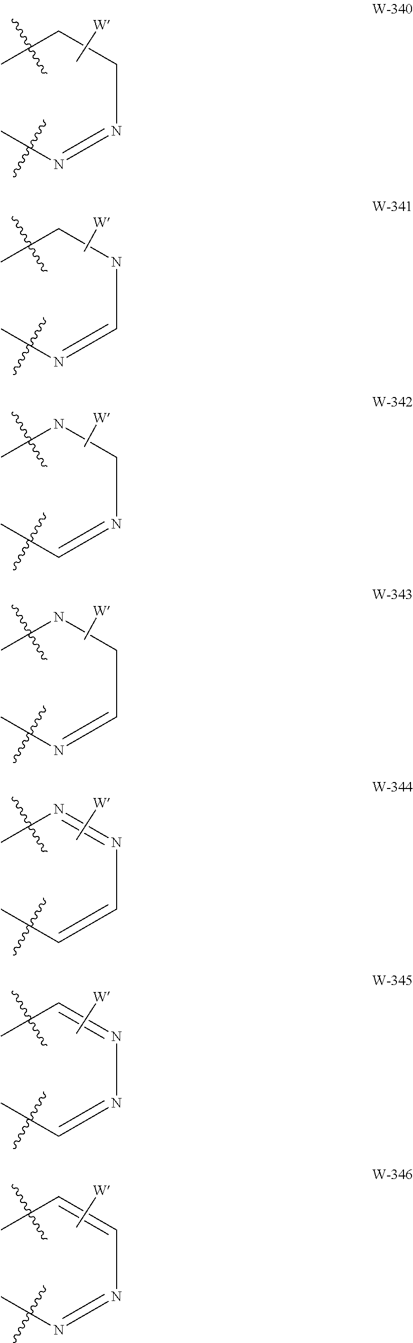 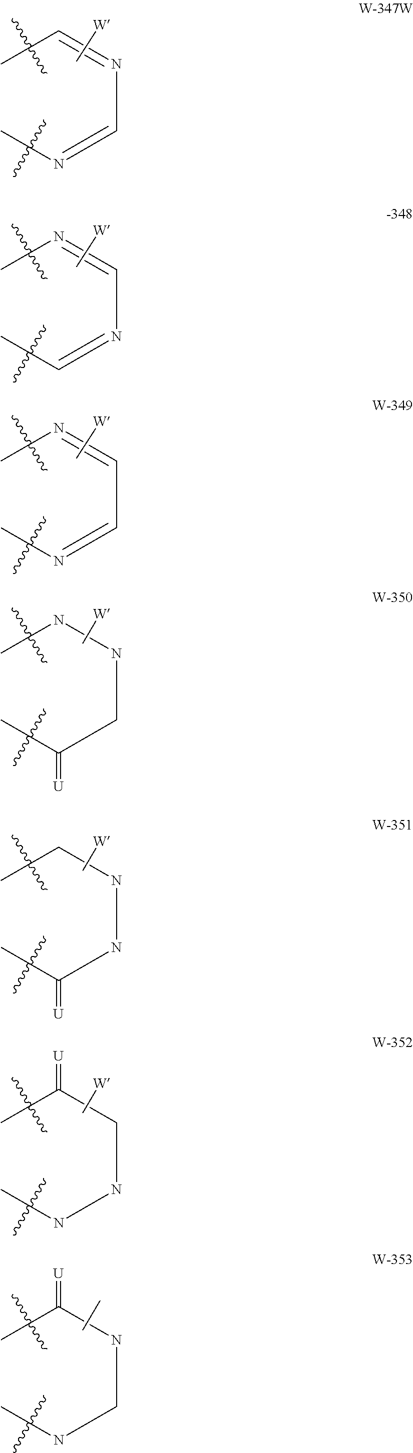 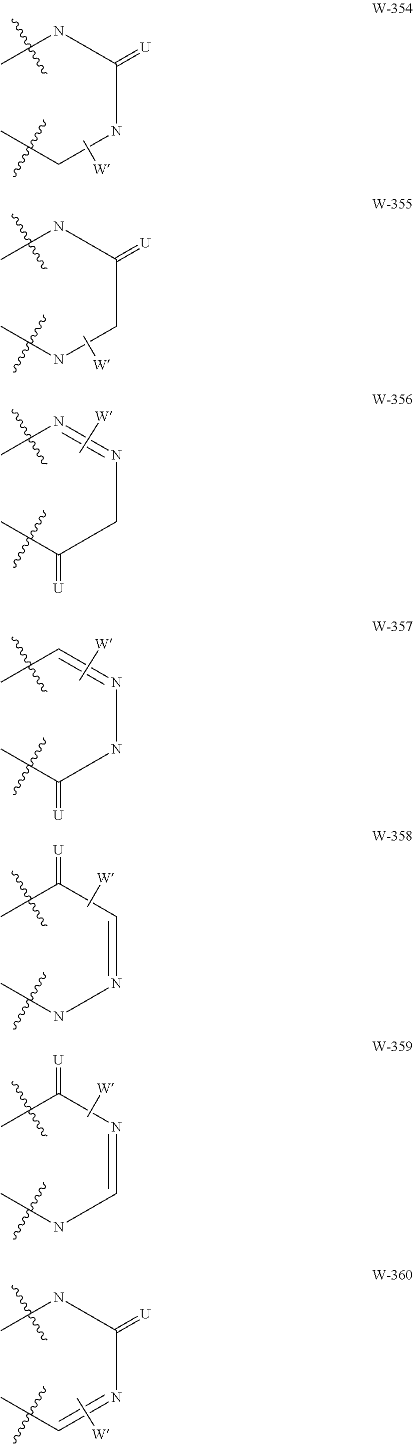 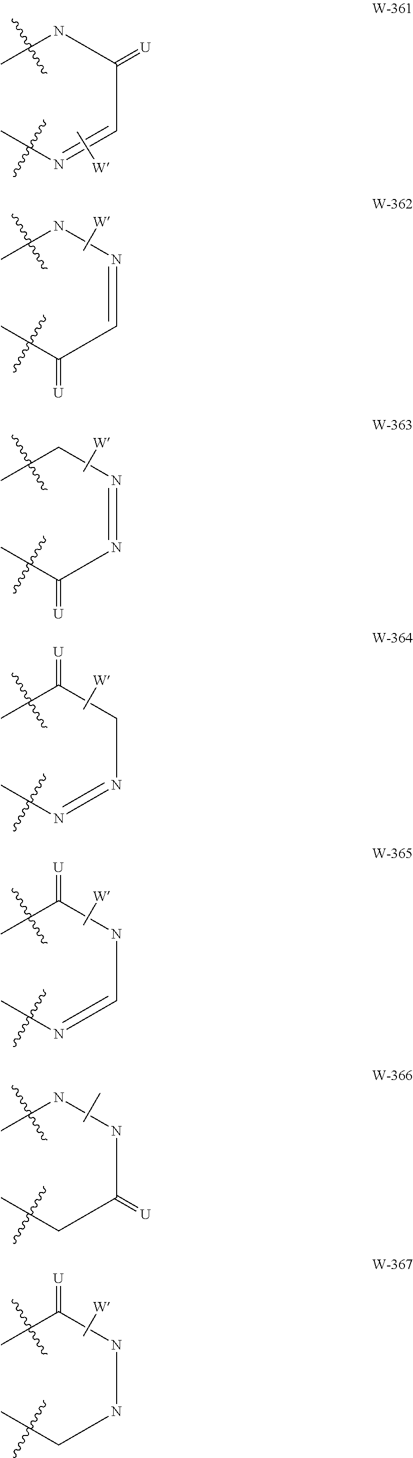 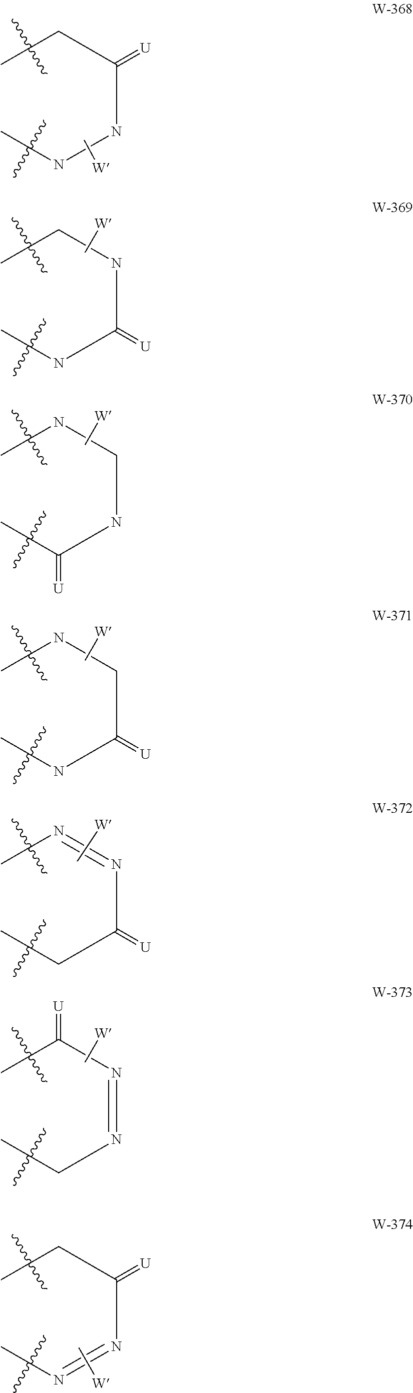 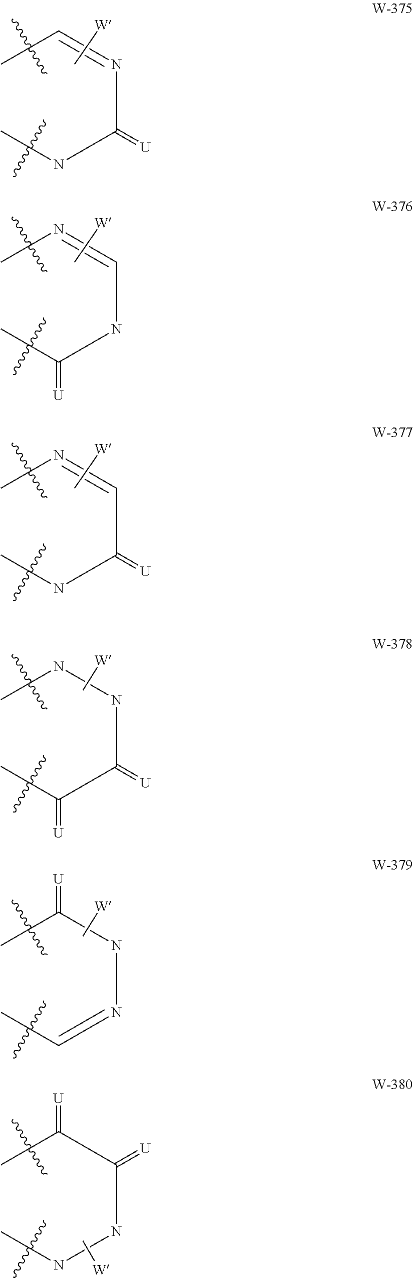 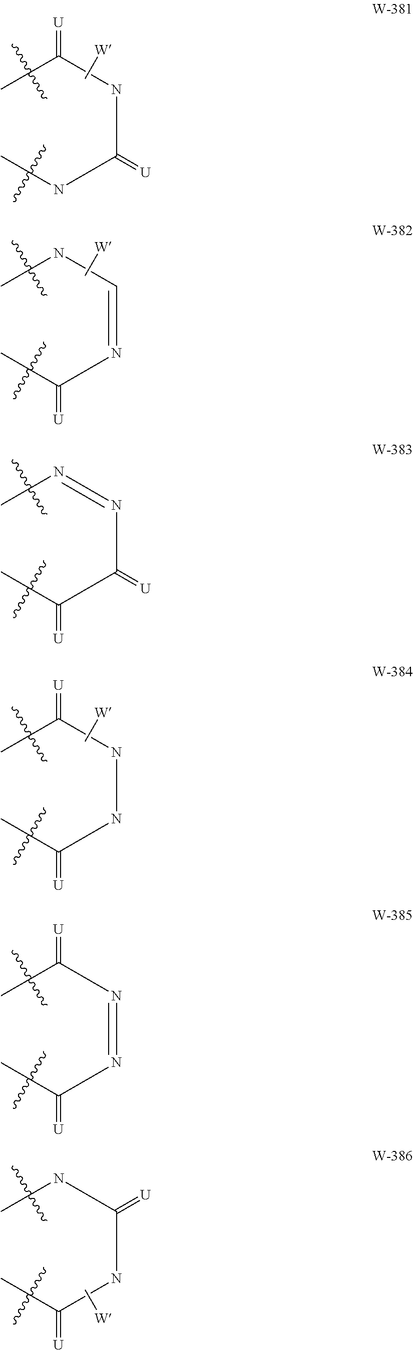 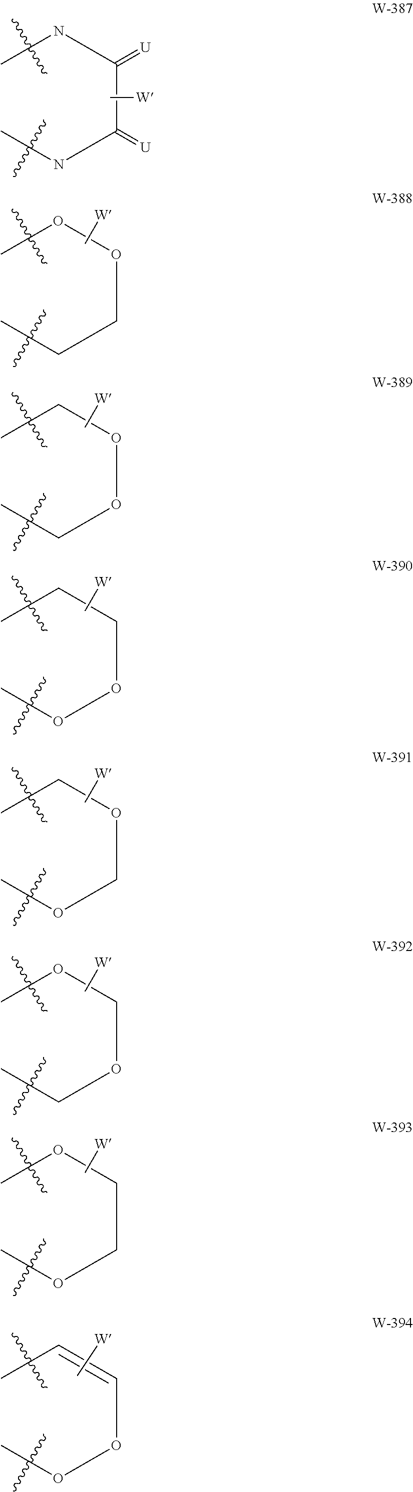 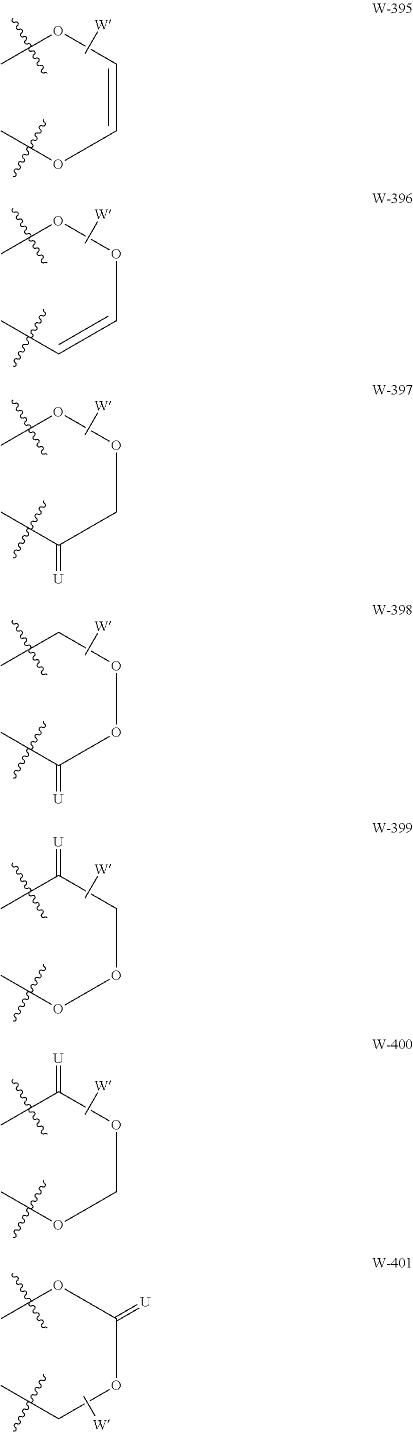 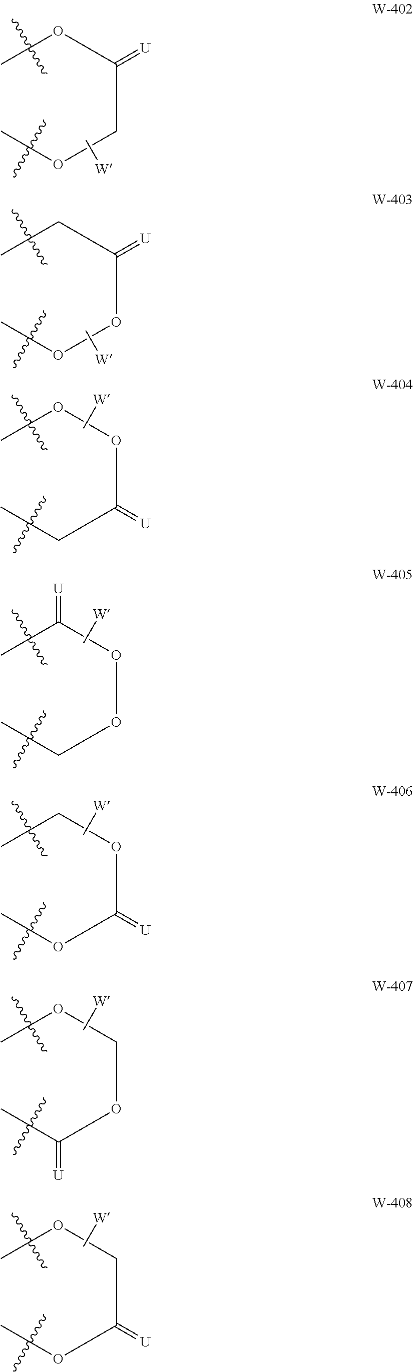 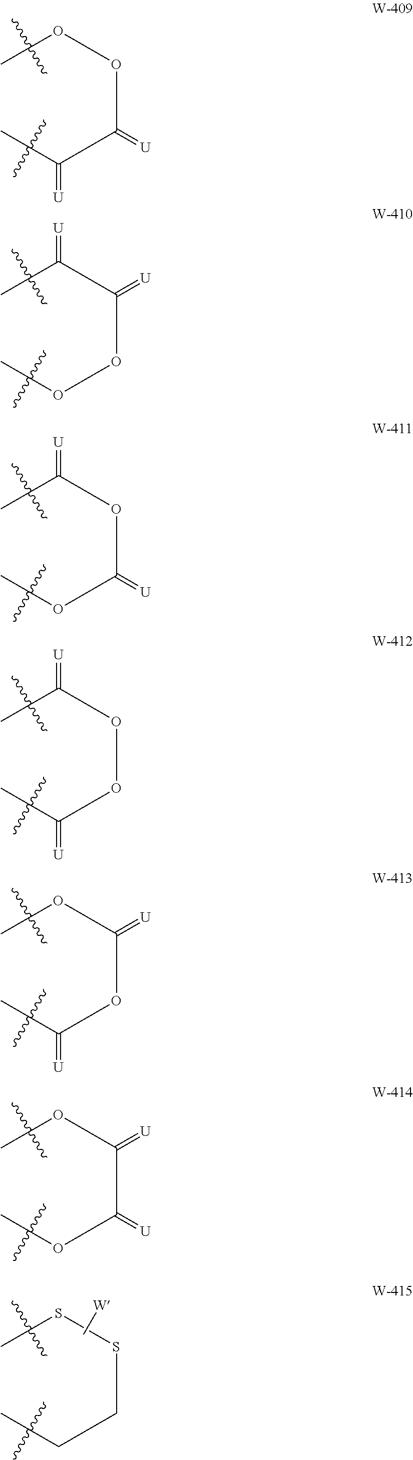 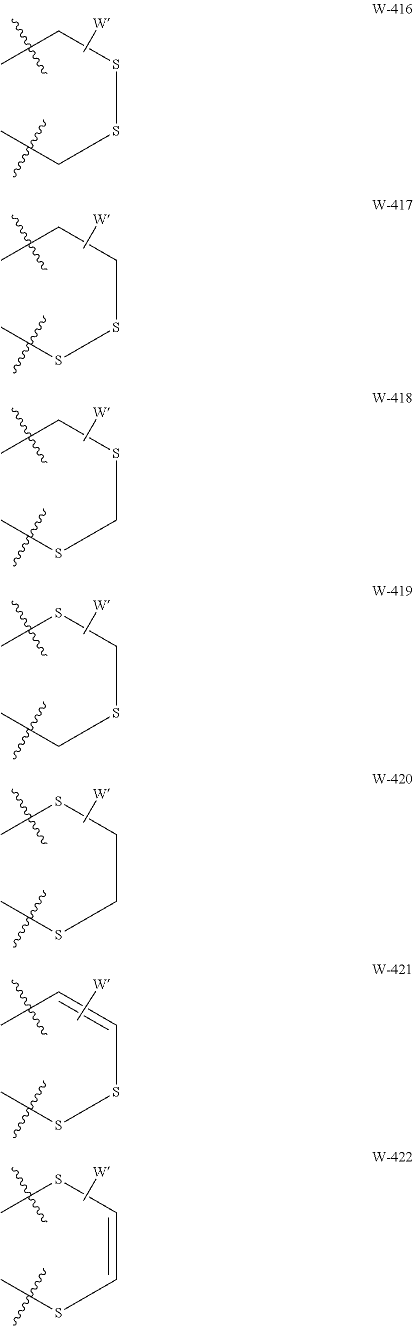 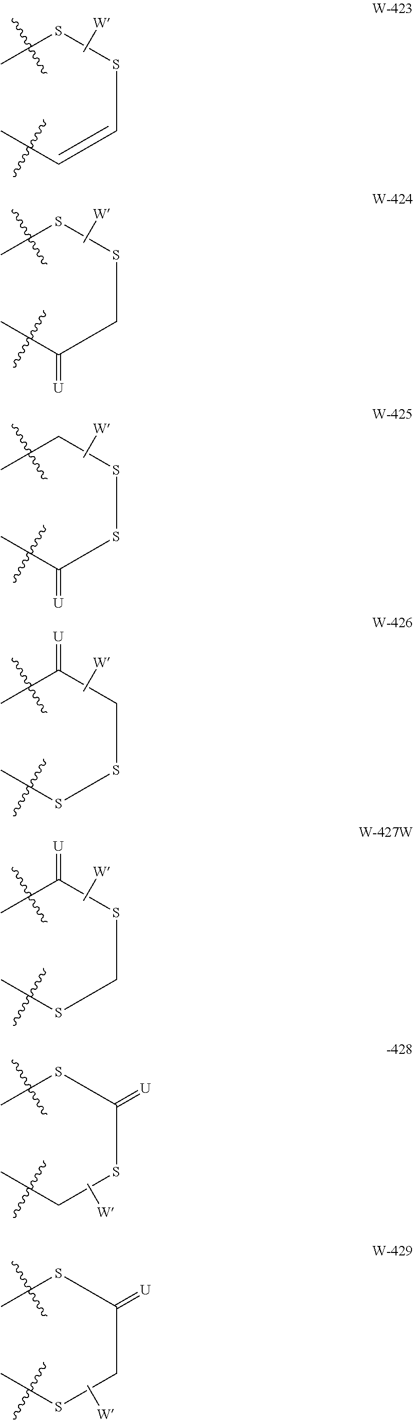 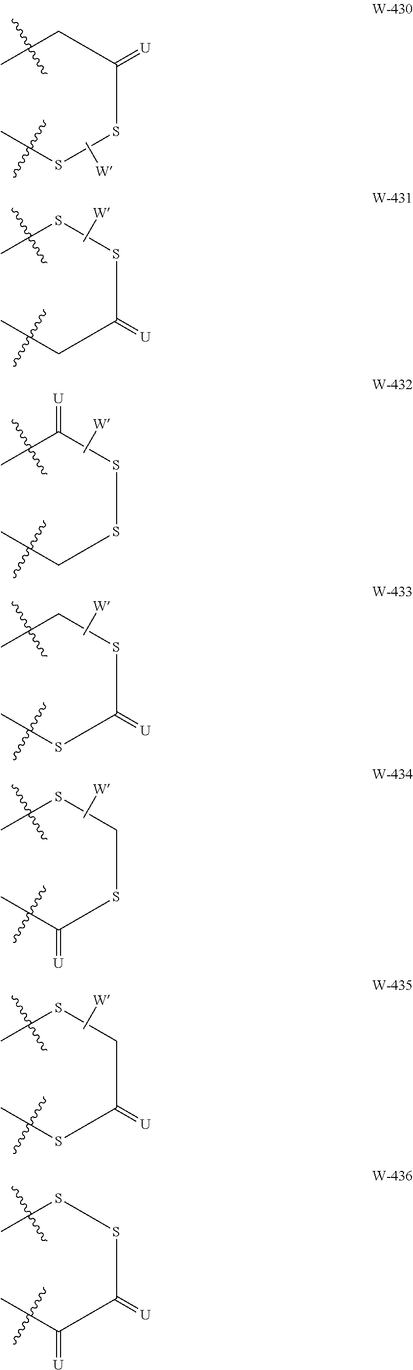 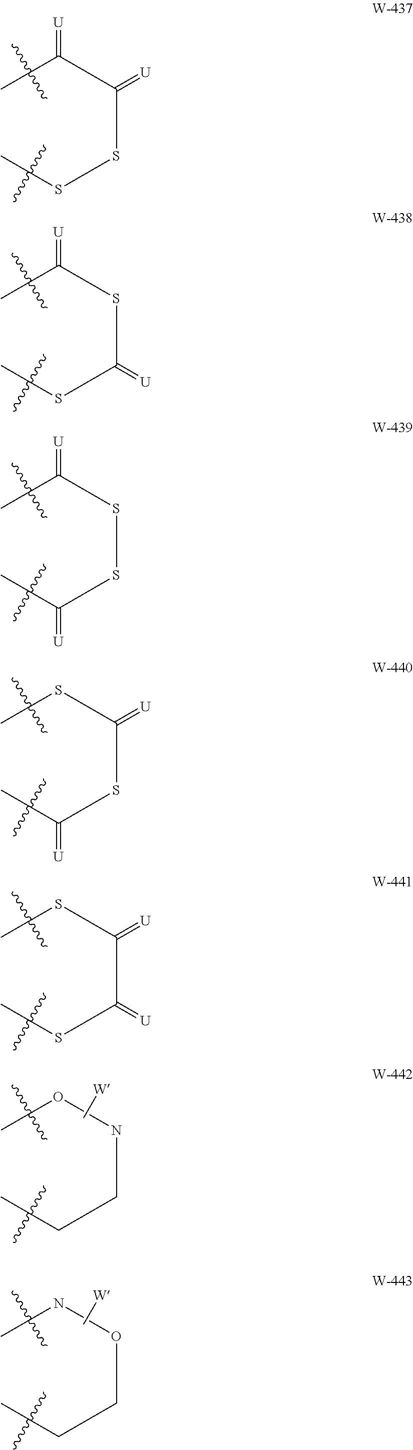 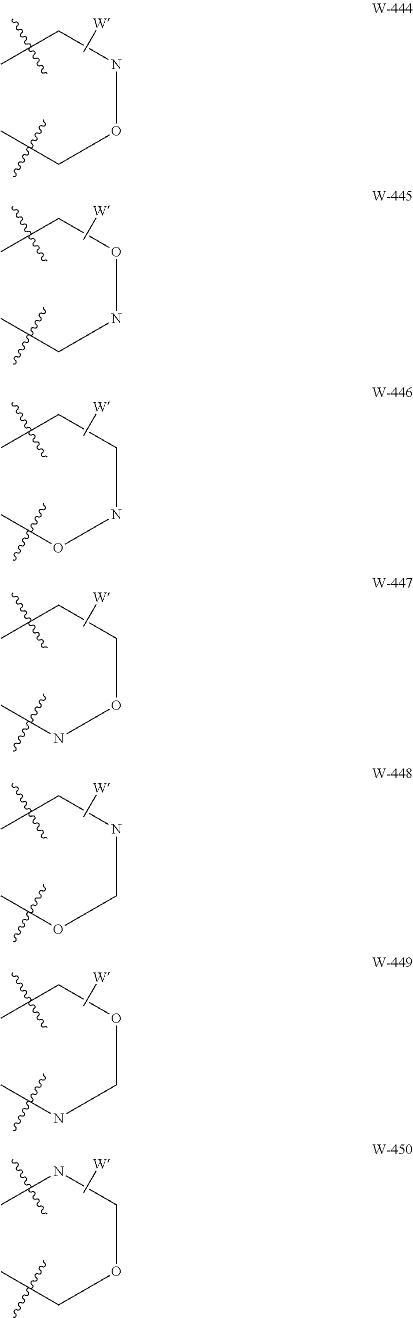 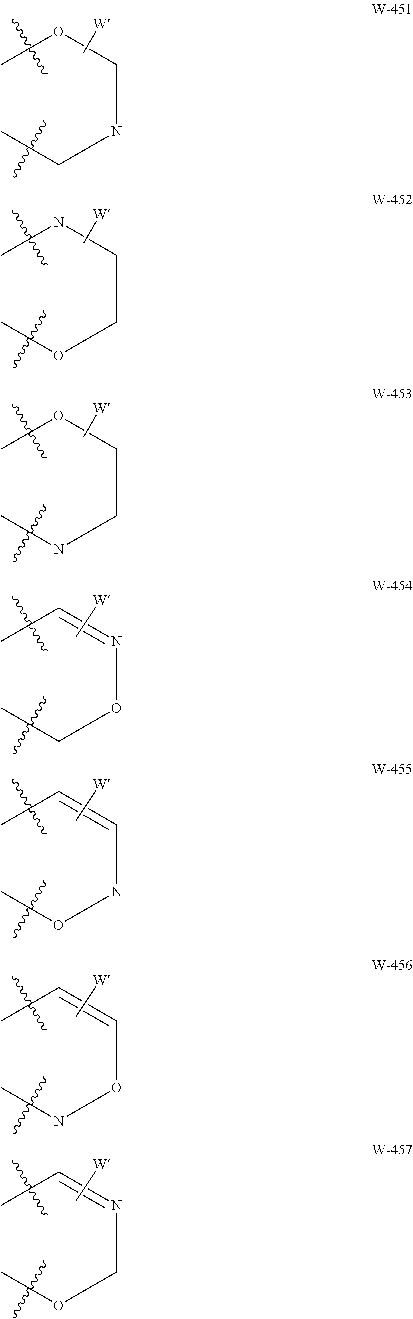 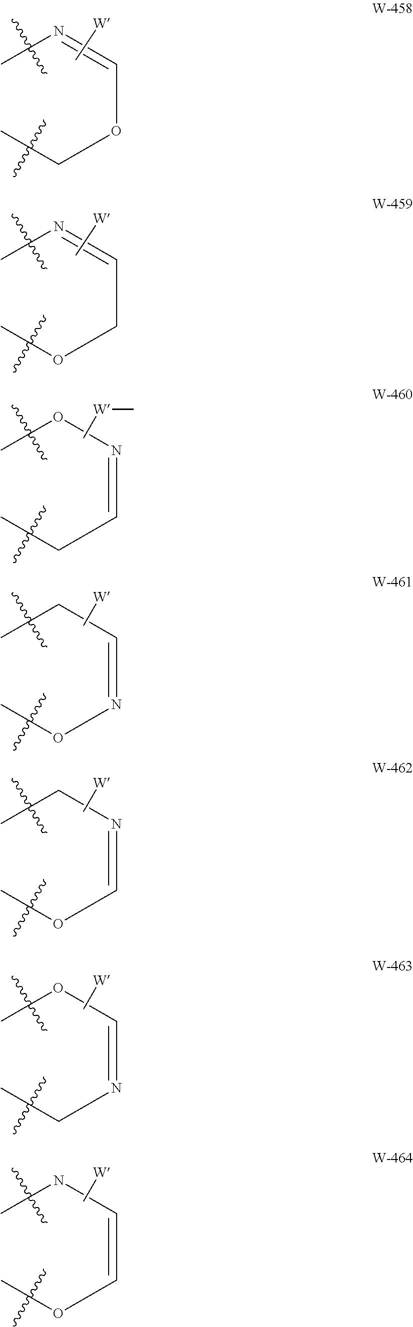 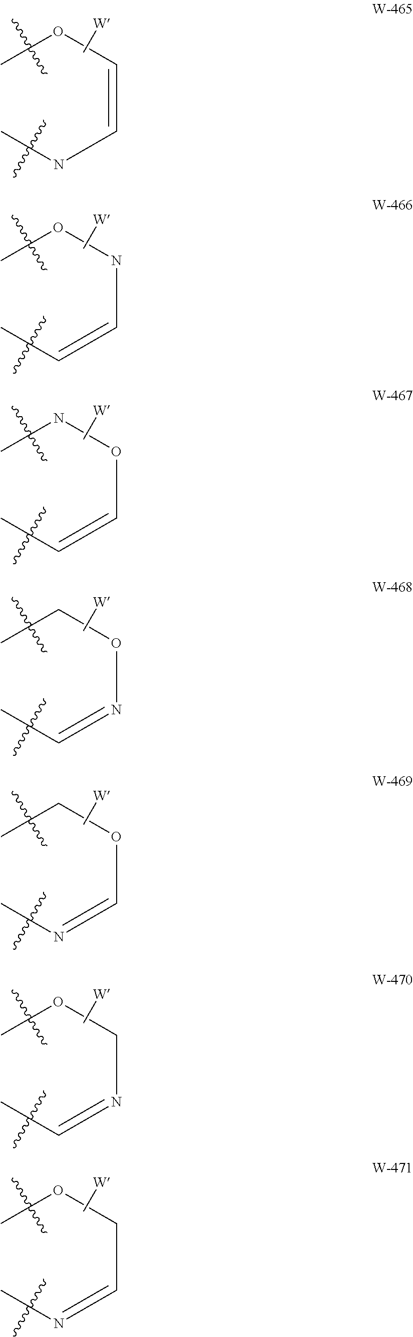 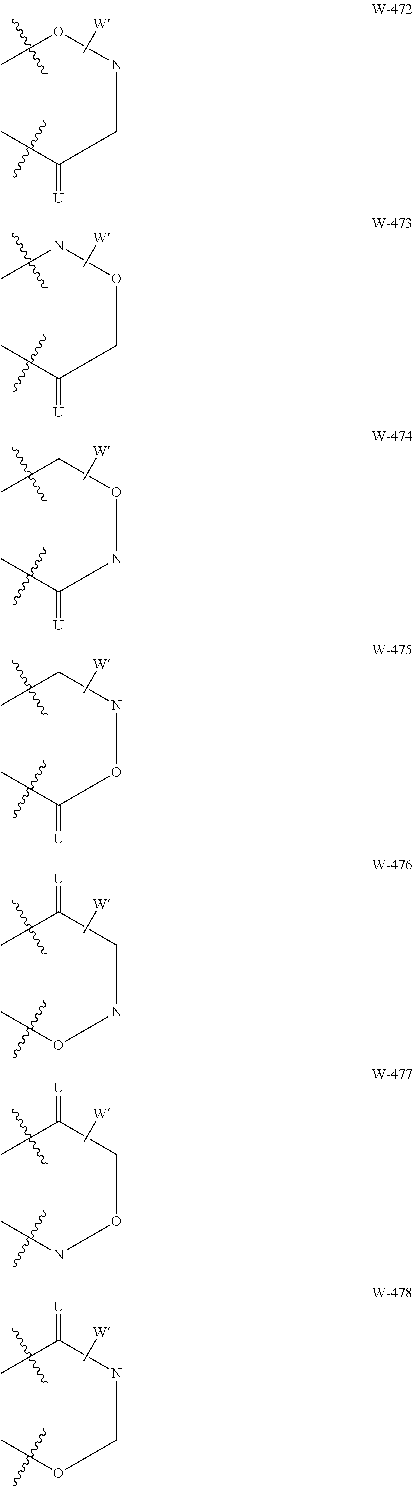 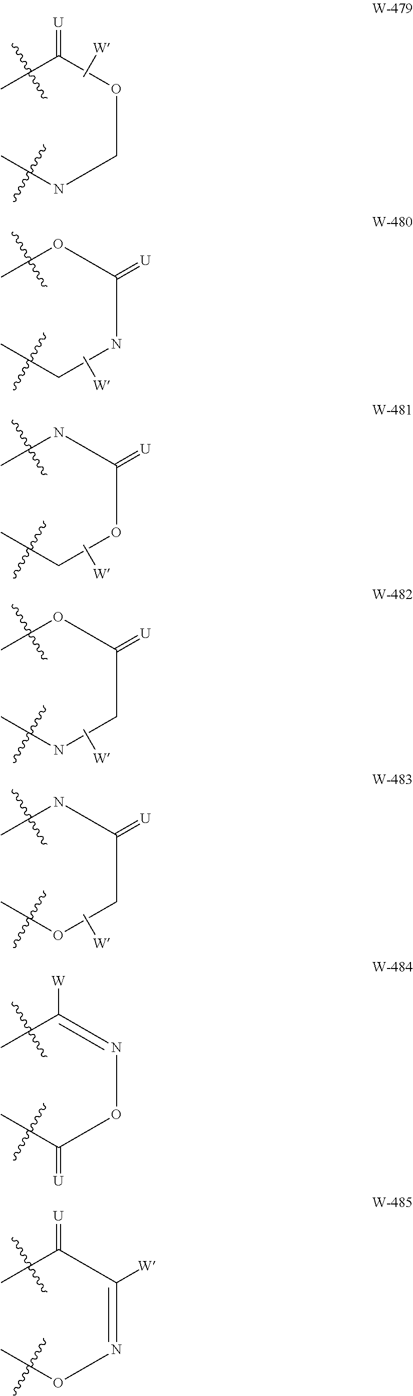 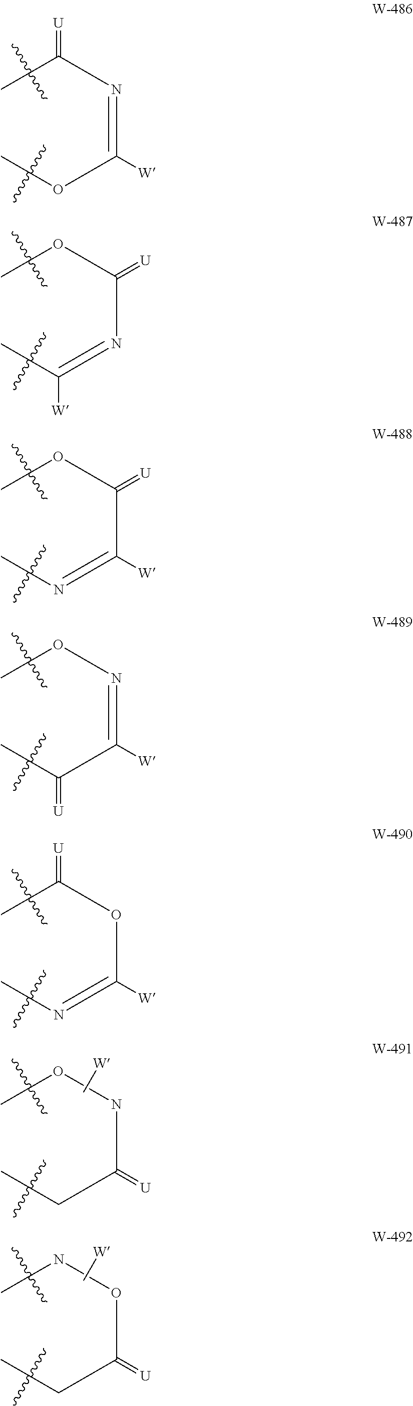 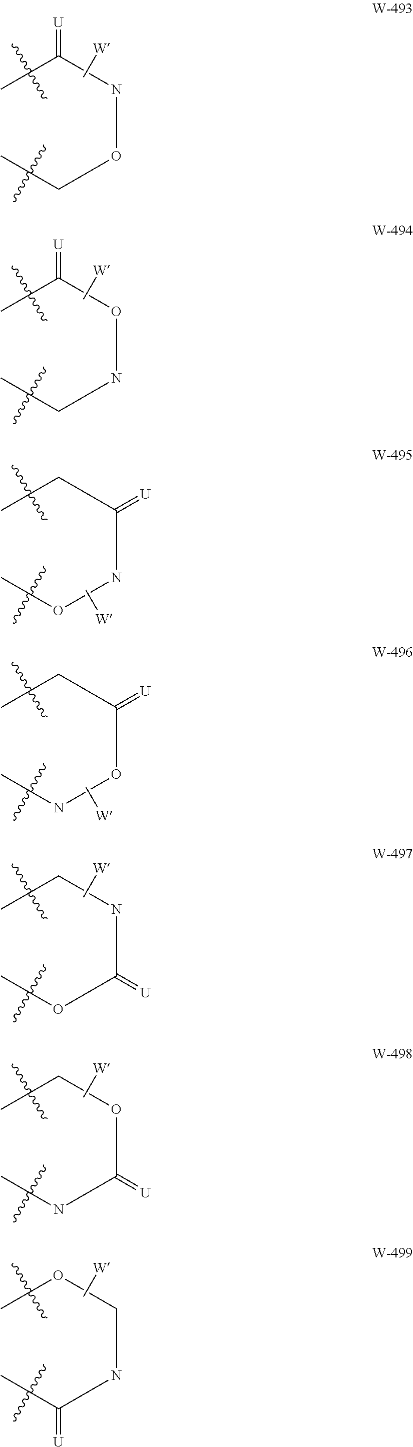 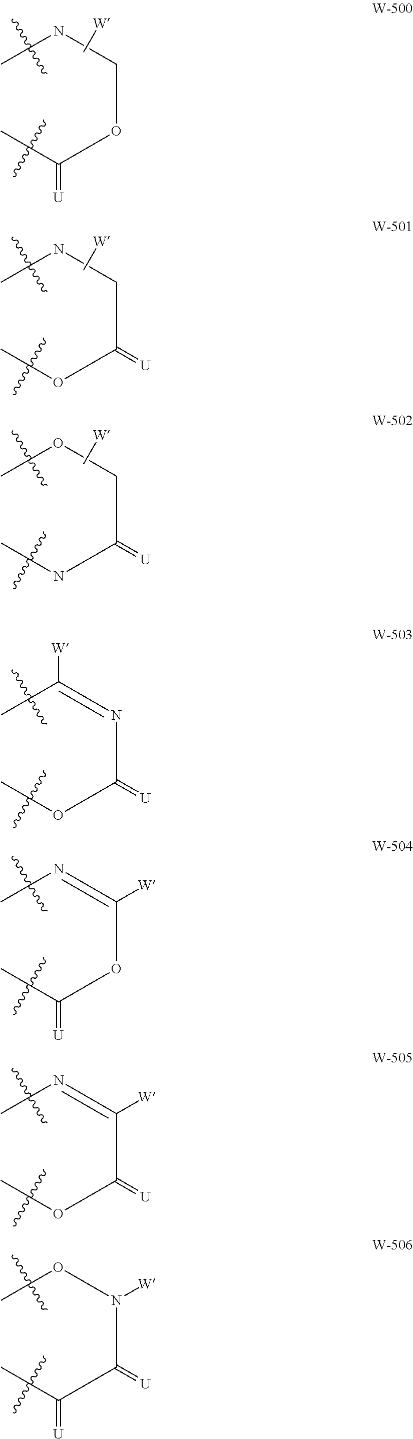 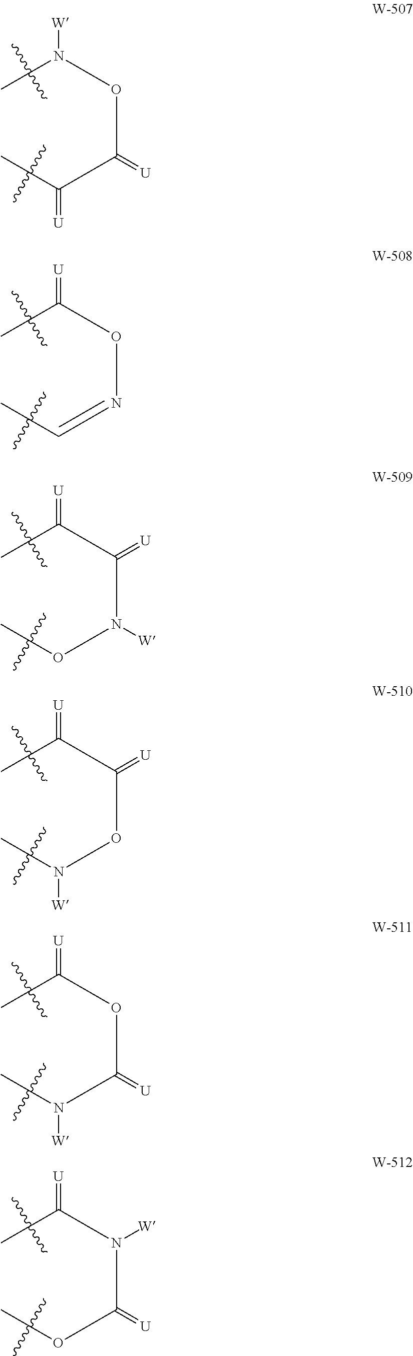 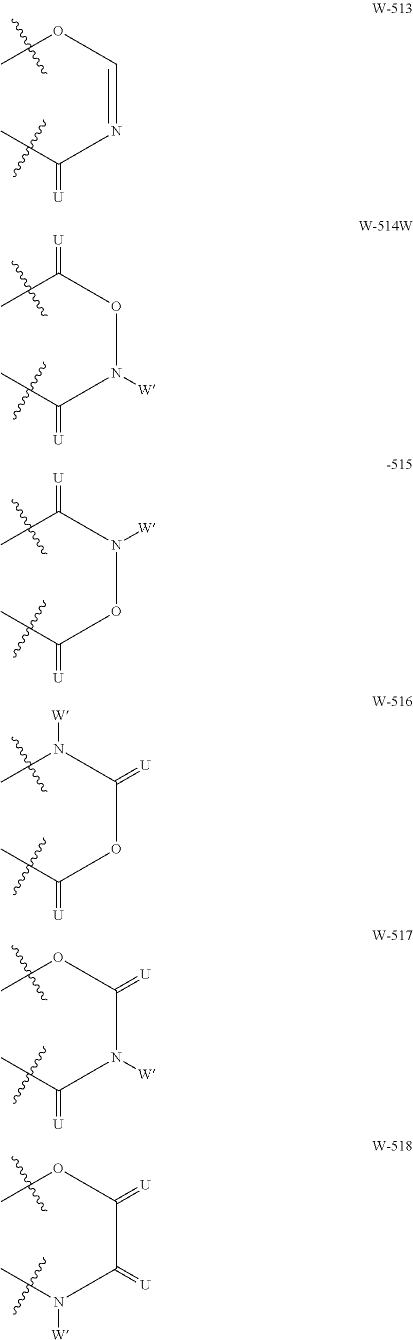 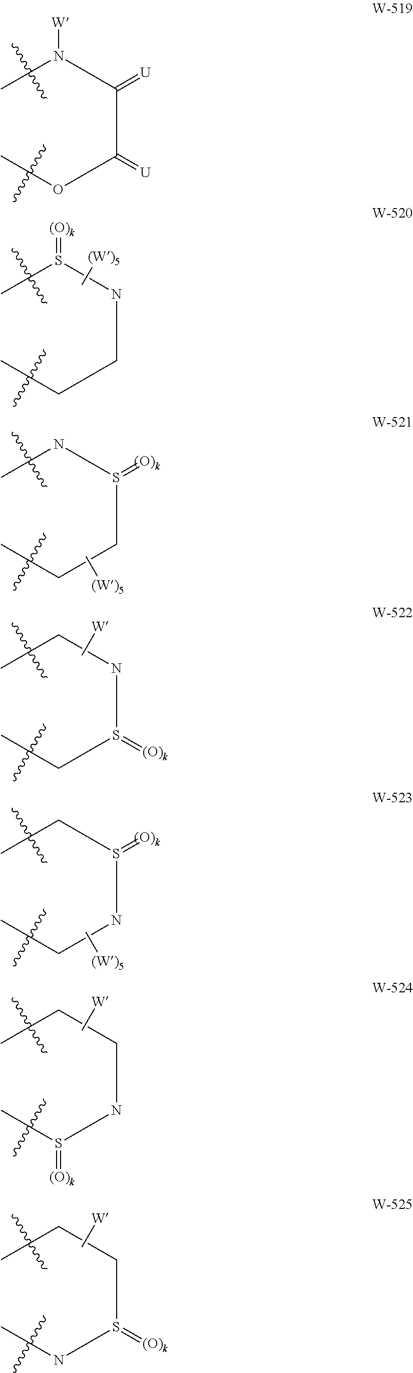 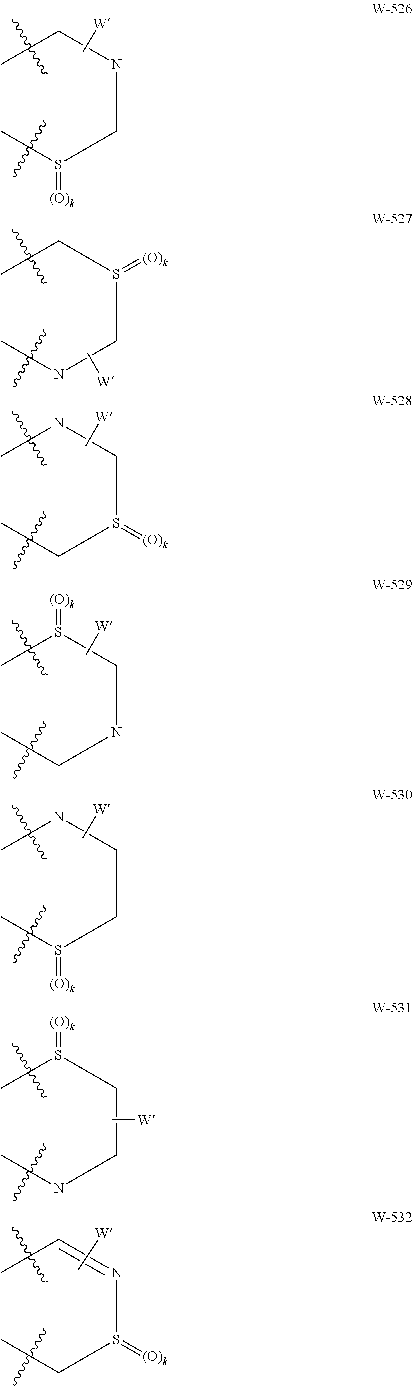 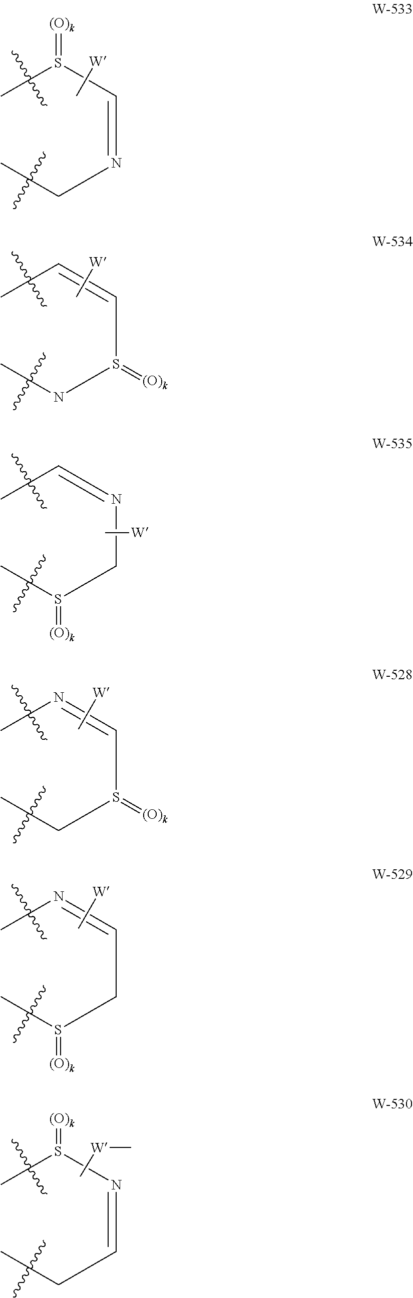 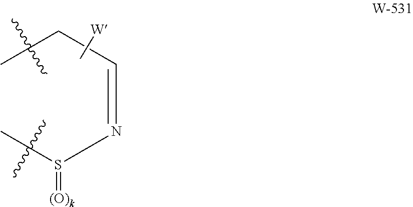 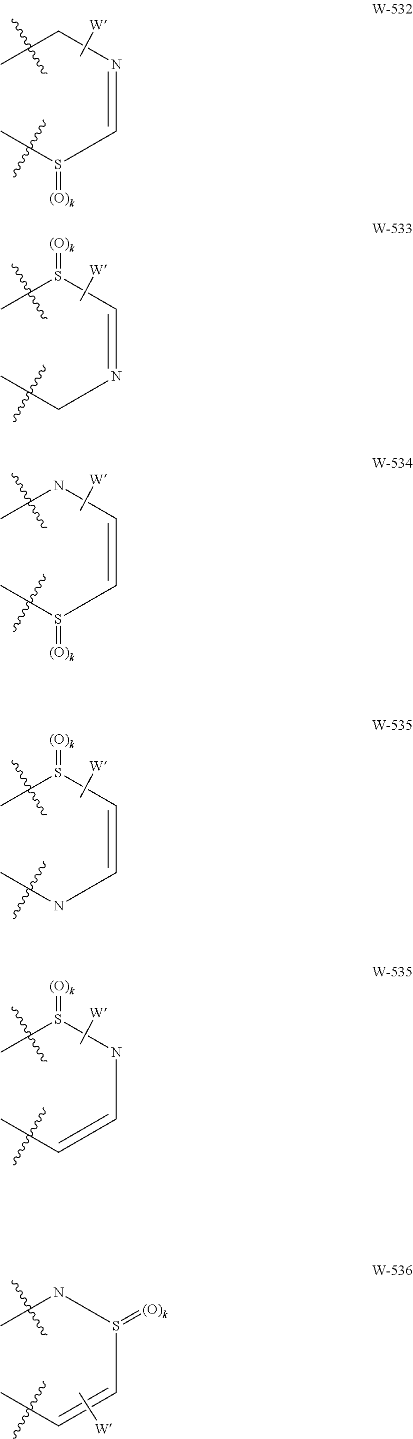 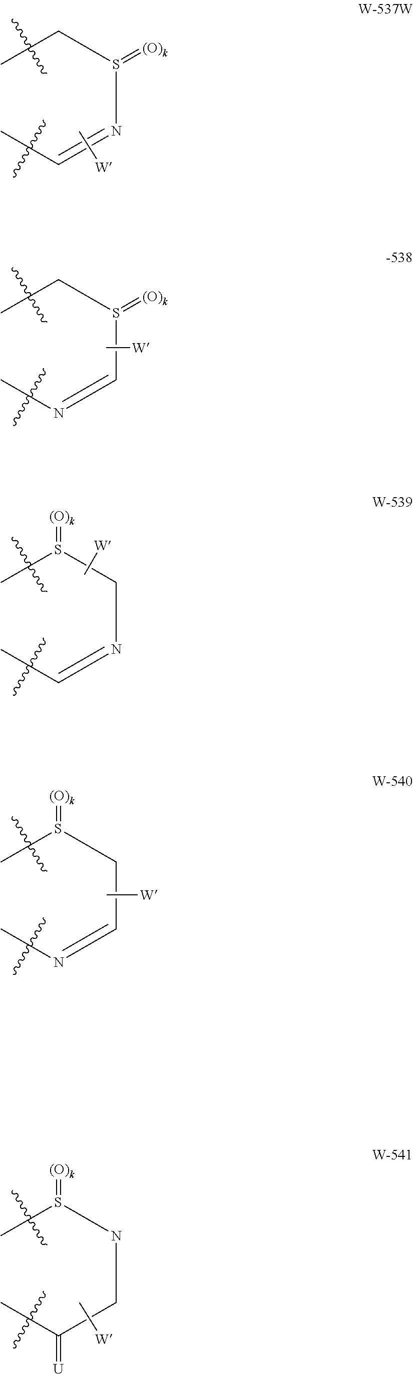 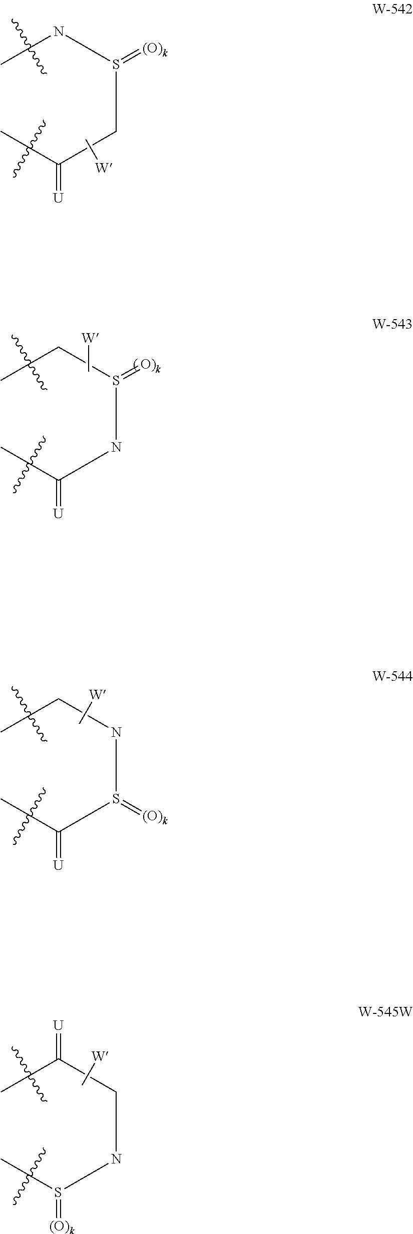 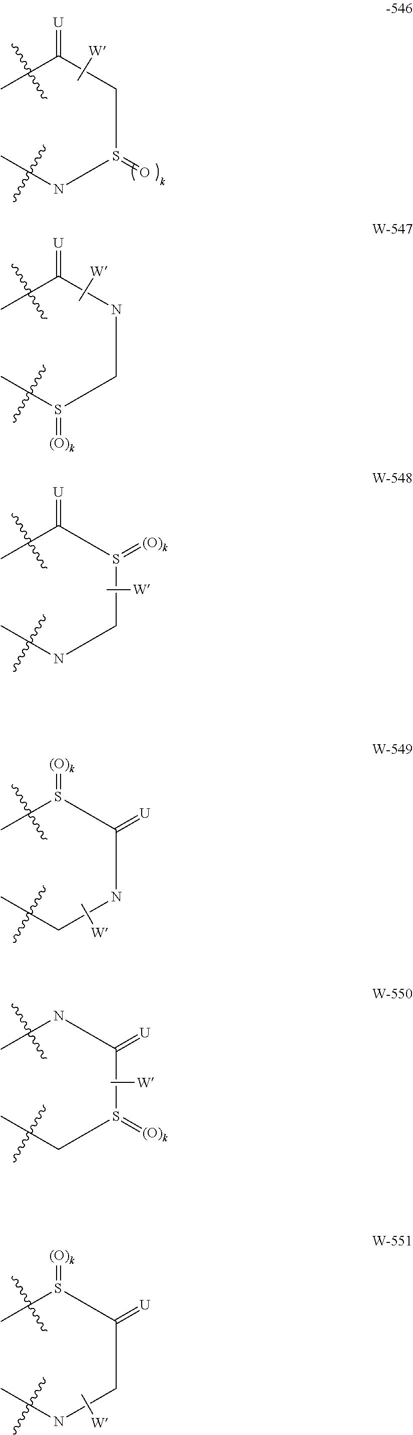 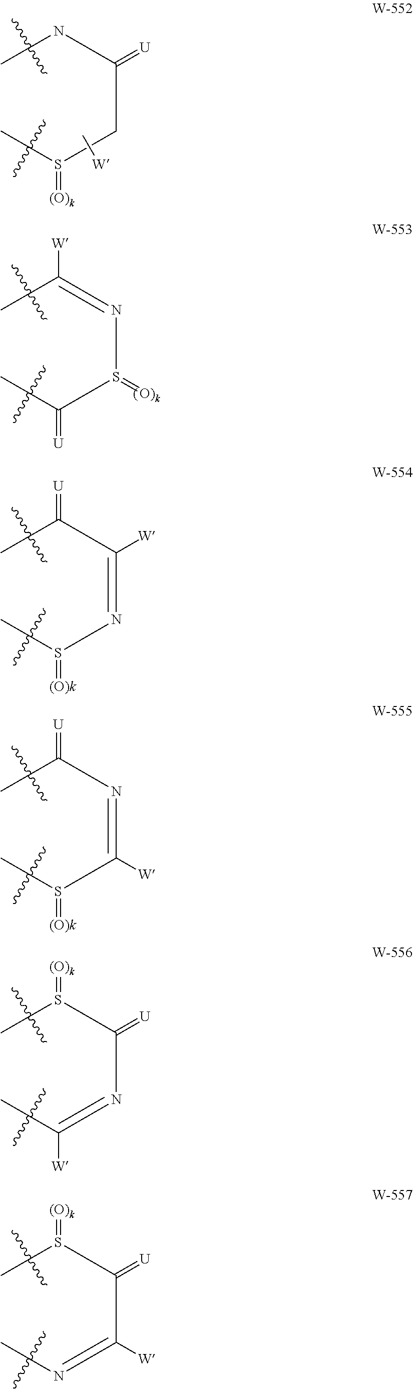 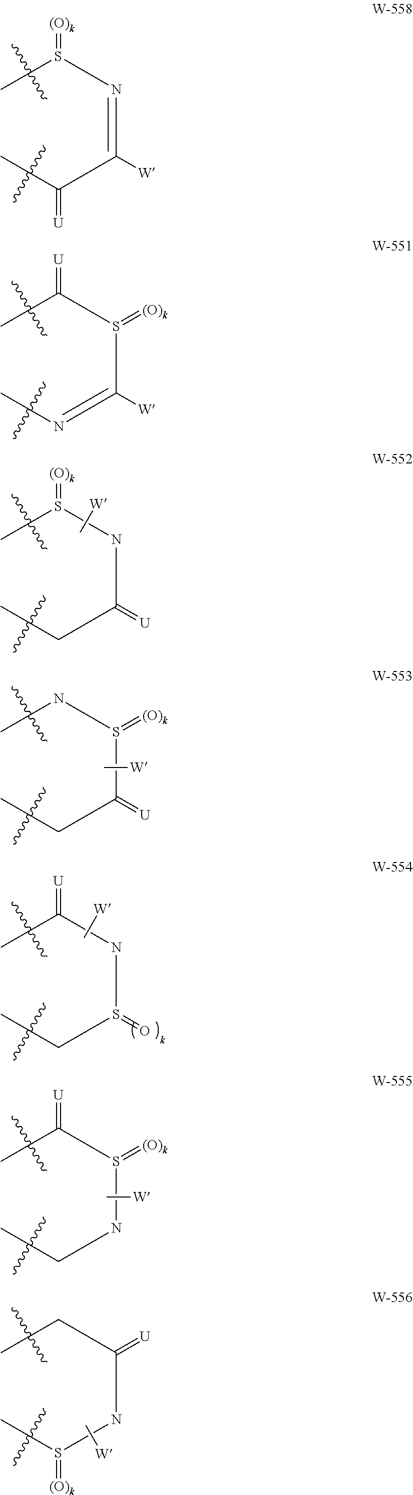 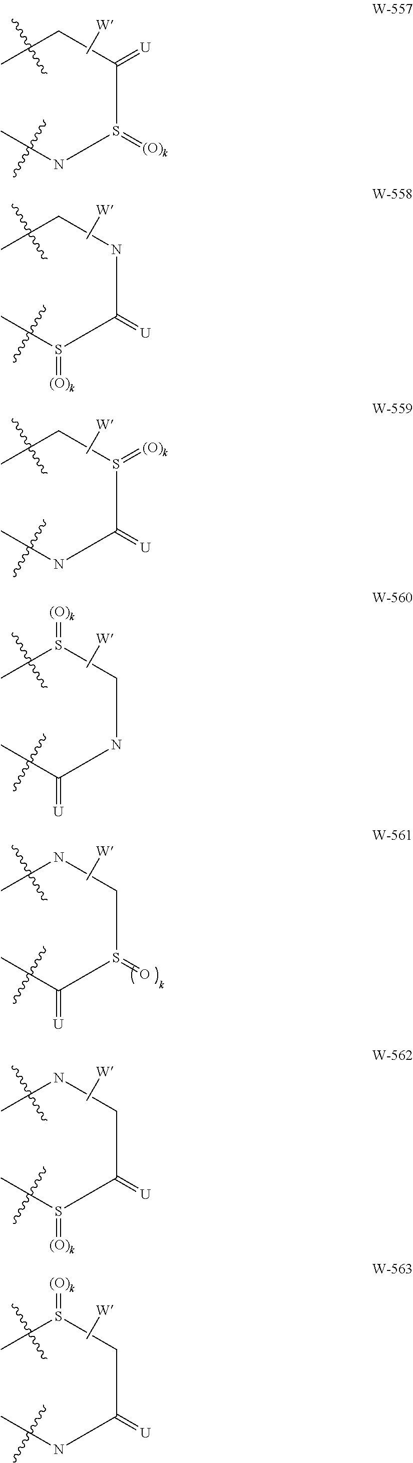 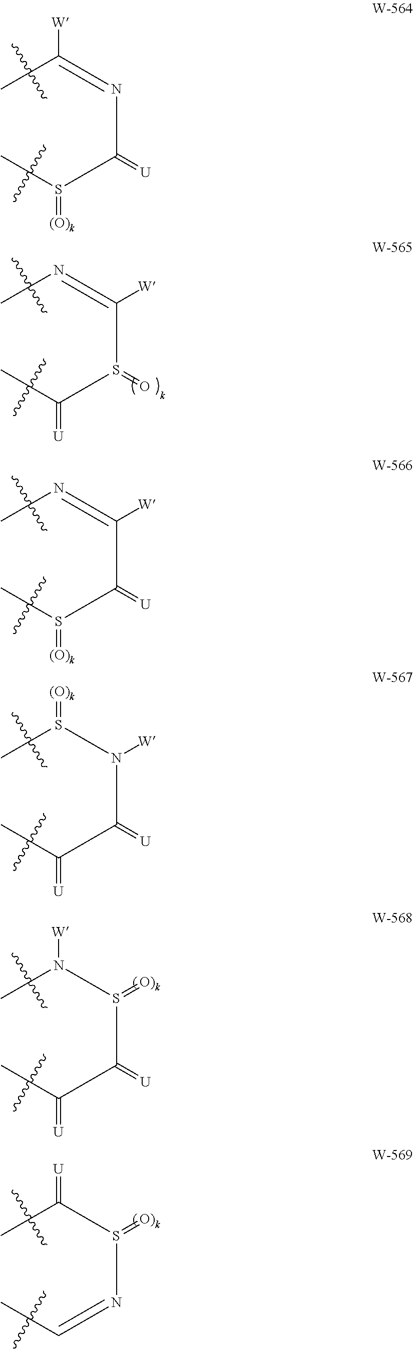 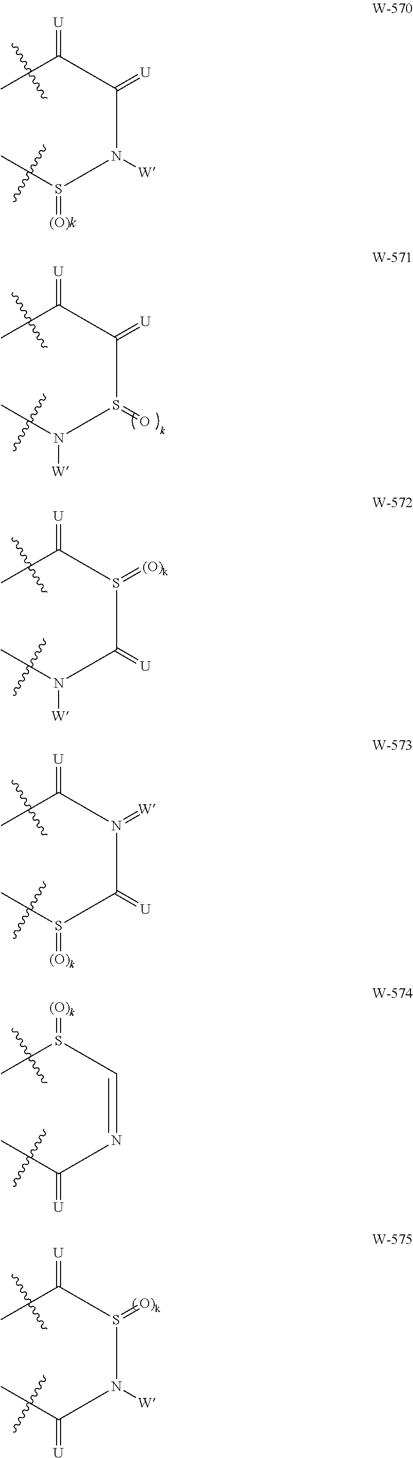 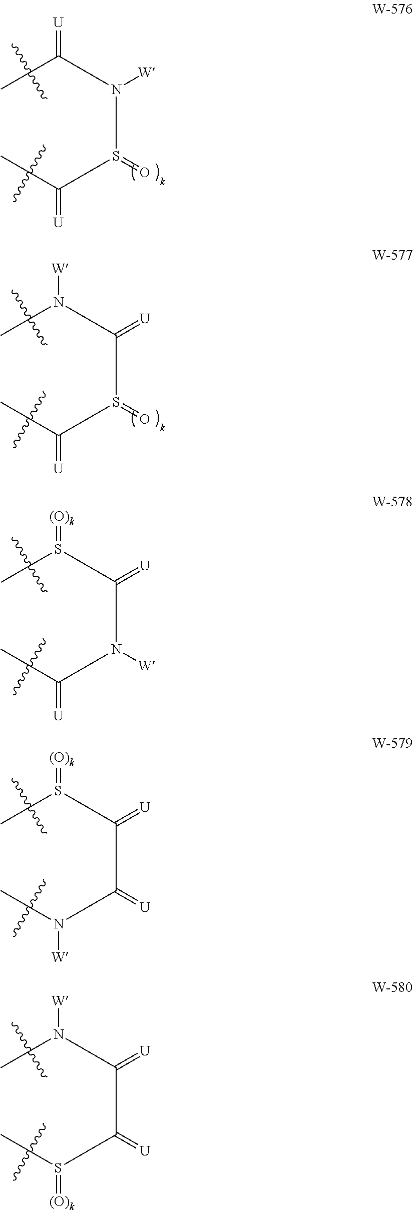
wherein
U is CH₂, O, S, N—R³, or N—R⁴;
k is 0, 1 or, 2;
W′ is O⁻, R³, OR³, SR³, NHR³, N(R³)₂, N(R³)N(R³)R³, N(R⁴)N(R³)R³, N(R³)OR³, R⁴, NR⁴, or N₃;
R³ each independently is hydrogen, hydroxy thiol, amino, cyano formyl, halogen, nitro, C₁₋₆ alkyl, C₂₋₁₂ alkoxyalkyl, C₂₋₁₂ haloalkoxyalkyl, C₂₋₆ alkenyl, C₂₋₁₂ alkynyl, C₃₋₈ cyclo-alkyl, C₄₋₁₂ alkylcycloalkyl, C₄₋₁₂ cycloalkylalkyl, C₁₋₆ haloalkyl, C₁₋₆ alkyl-carbonyl, C₁₋₆ alkylcarbonyl-C₁₋₆ alkyl, C₁₋₆ alkylcarbonyl-C₁₋₆ alkylcarbonyl, C₁₋₆ haloalkylcarbonyl, C₁₋₆ alkoxycarbonyl, C₁₋₆ alkylsulfenylcarbonyl, C₁₋₆ haloalkylsulfenylcarbonyl, aminocarbonyl, C₁₋₆ alkylaminocarbonyl, C₁₋₆ haloalkylaminocarbonyl, C₁₋₆ hydroxyalkylaminocarbonyl, C₂₋₁₂ dialkyl-amino-carbonyl, C₂₋₆ di(haloalkyl)aminocarbonyl, C₂₋₆ alkenylaminocarbonyl, C₂₋₆ alkynylaminocarbonyl, C₁₋₆ alkyl-thiocarbonyl, C₃₋₆ cycloalkylcarbonyl, C₄₋₁₂ cycloalkylalkyl-carbonyl, C₃₋₆ cycloalkyl-thiocarbonyl, C₄₋₁₂ cycloalkyl-alkyl-thiocarbonyl, C₁₋₆ haloalkyl-thiocarbonyl, C₁₋₆ alkylamino-thiocarbonyl, C₃₋₆ cycloalkylamino-carbonyl, C₄₋₁₂ cycloalkylalkylamino-carbonyl, C₃₋₆ cyclo-alkylamino-thiocarbonyl, C₄₋₁₂ cycloalkylalkylaminothiocarbonyl, C₁₋₆ haloalkylamino-thiocarbonyl, C₂₋₁₂ dialkylamino-thiocarbonyl, C₃₋₆ cyclo-alkyloxy-carbonyl, C₄₋₁₂ cycloalkylalkyloxy-carbonyl, C₁₋₆ haloalkoxy-carbonyl, C₁₋₆ alkylsulfonyl, C₁₋₆ haloalkylsulfonyl, phenylsulfonyl, R⁴—C₁₋₆ alkyl, R⁴-carbonyl, R⁴-thiocarbonyl, R⁴—C₁₋₆ alkylcarbonyl, R⁴—C₁₋₆alkyl-thiocarbonyl, R⁴-oxycarbonyl, R⁴—C₁₋₆alkyloxy-carbonyl, R⁴-aminocarbonyl, R⁴-amino-thiocarbonyl, R⁴—C₁₋₆ alkylamino-carbonyl, or R⁴—C₁₋₆ alkylamino-thiocarbonyl; and
R⁴ is phenyl or a 5- or 6-membered saturated or unsaturated heterocyclic ring;

3. The compound according to claim 2, wherein
R⁴ is selected from R⁴-1 to R⁴-83 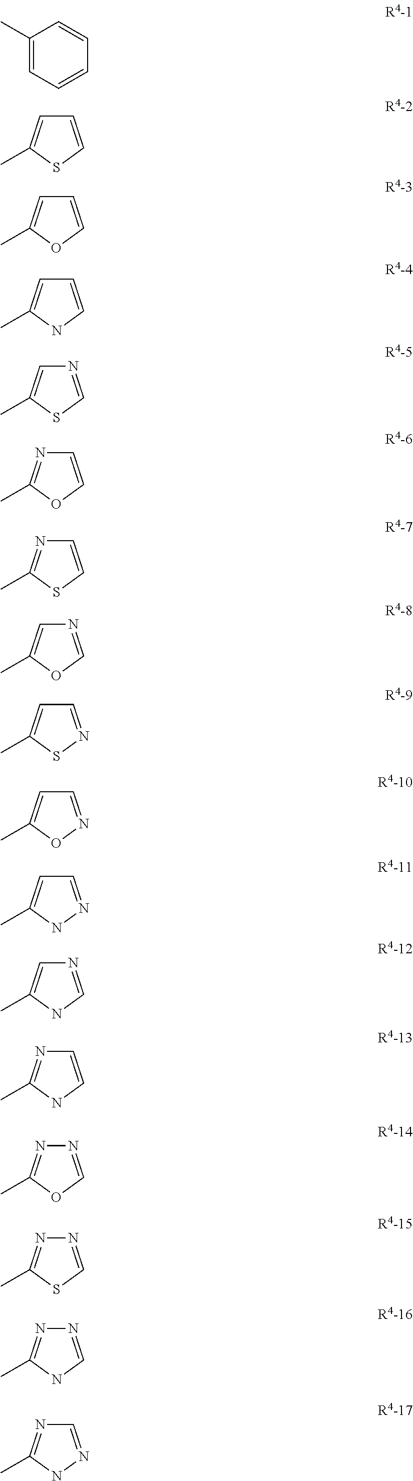 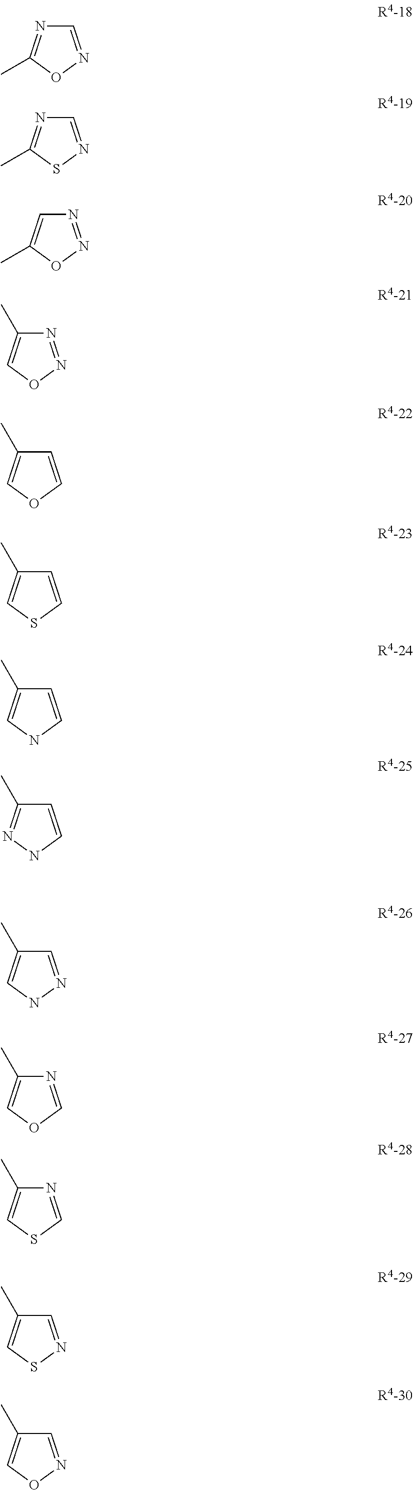 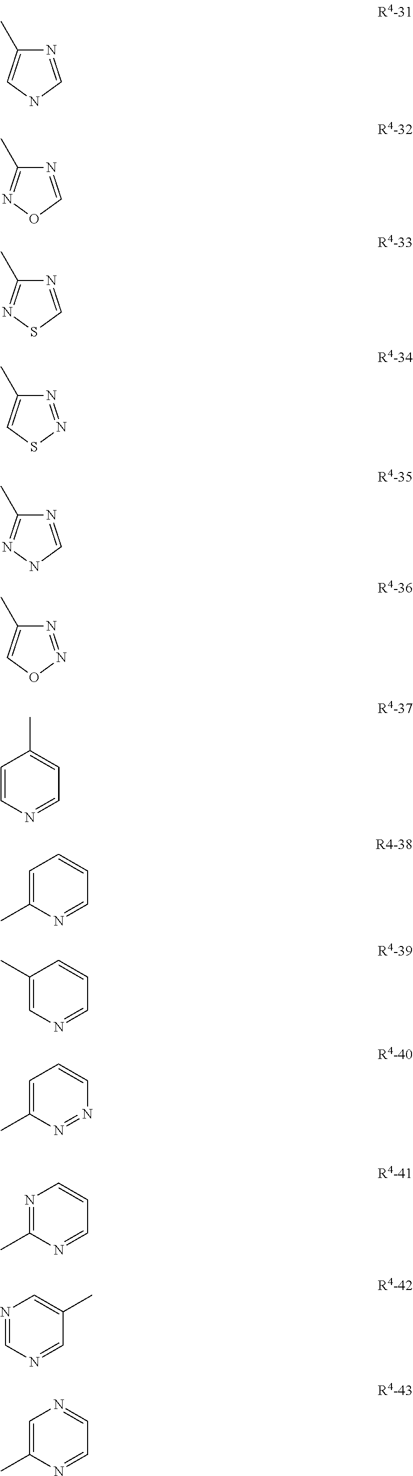 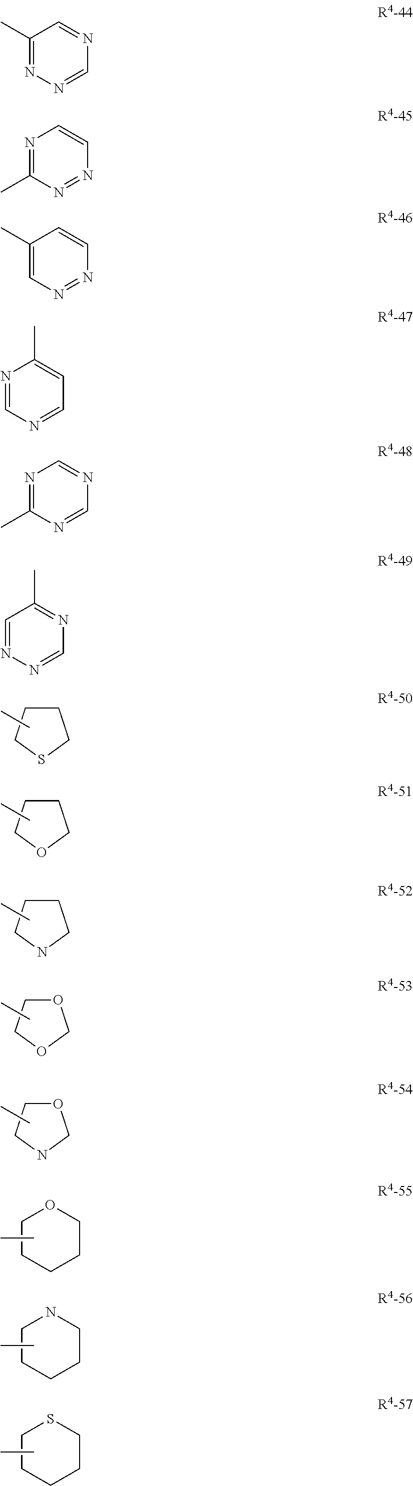 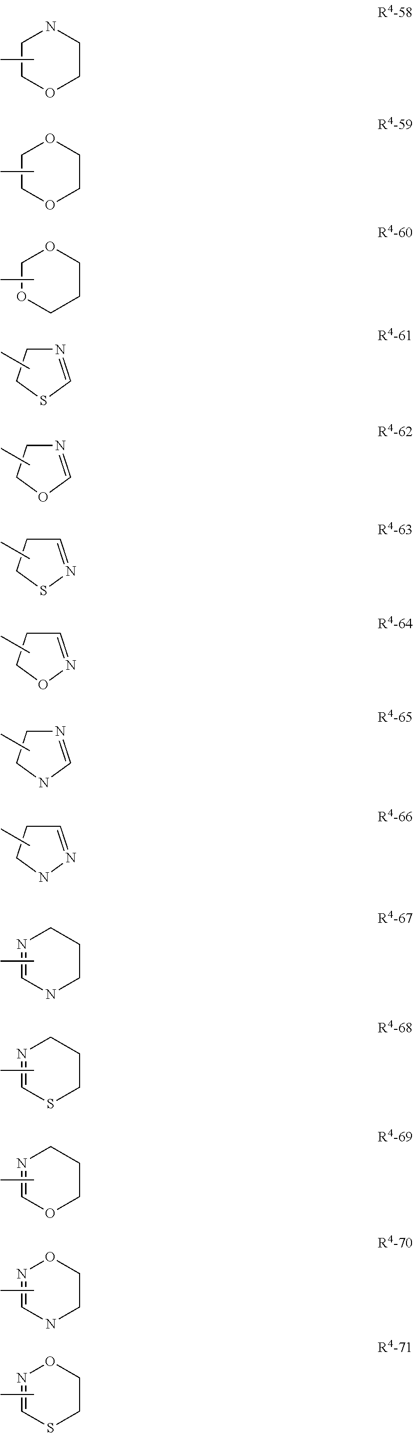 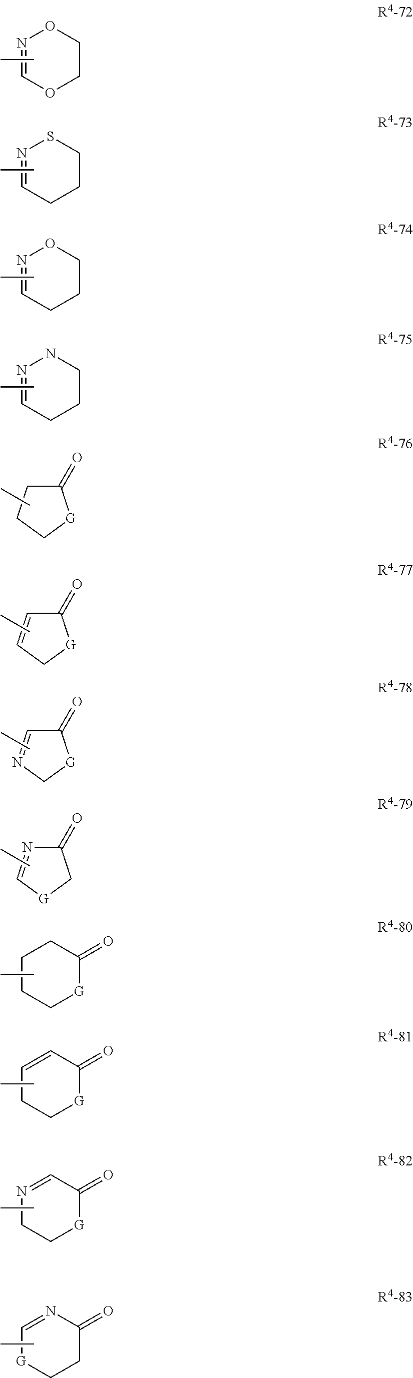
wherein
G represents is O, S, or N, and
R⁴-1 to R⁴-83 are optionally substituted with with one or more substituents selected among from hydrogen, halogen, cyano, nitro, C₁₋₆ alkyl, C₁₋₆ haloalkyl, C₃₋₆ cycloalkyl, C₃₋₆ halocycloalkyl, C₁₋₆ alkoxy, C₁₋₆haloalkoxy, C₁₋₆ alkylsulfenyl, C₁₋₆haloalkylsulfenyl, C₁₋₆ alkyl-sulfinyl, C₁₋₆ haloalkylsulfinyl, C₁₋₆ alkylsulfonyl, C₁₋₆haloalkylsulfonyl, C₁₋₆alkylamino, C₁₋₆haloalkylamino, aminocarbonyl, C₁₋₆ alkylamino-carbonyl, C₂₋₁₂ dialkylamino-carbonyl, C₁₋₆alkoxycarbonyl, phenyl, or pyridyl.

4. A pesticide comprising at least one compound according to claim 1.

5. A method for controlling animal pests comprising applying at least one compound according to claim 1 to animal pests, their habitat, or combinations thereof.

6. A method for treating seed comprising applying at least one compound according to claim 1 to seed of a conventional or transgenic plant.

7. A pharmaceutical composition comprising at least one compound according to claim 1.

8. A method for controlling parasites comprising applying at least one compound according to claim 1 to a parasite in or on an animal.

9. The compound of claim 1, wherein X is chloro, bromo, iodo, fluoro, nitro, cyano, hydroxy, thiol, amino, C₁₋₆ alkyl, C₁₋₆ haloalkyl, C₁₋₆ alkoxy, C₁₋₆ haloalkoxy, C₁₋₆ alkylsulfenyl, C₁₋₆ alkylsulfenyl, C₁₋₆ alkylsulfonyl, C₁₋₆haloalkylsulfenyl, C₁₋₆ haloalkylsulfinyl, C₁₋₆haloalkylsulfonyl, C₁₋₆ alkyl-amino, C₂₋₁₂ dialkylamino, C₁₋₆ acylamino, C₁₋₆ alkoxy-carbonylamino, C₁₋₆haloalkoxy-carbonylamino, C₁₋₆alkylsulfonylamino, or C₁₋₆haloalkyl-sulfonylamino.

10. The compound of claim 1, wherein W¹, W², W³, and W⁴ each independently is a single bond, CH₂, CH, N⁺(O⁻)—, —S(O)—, —S(O)₂—, —O—S(O)—, O, S, C(R³)—R³, C—R³, C—R⁴, C(R³)—R⁴, C(R⁴)—R⁴, C—N(R³)—R³, C(R³)—N(R³)—N(R³)—R³, C—N(R³)—N(R³)—R³, C(R³)—N(R⁴)—N(R³)—R³, C—N(R⁴)—N(R³)—R³, C(R³)—N(R³)—OR³, C—N(R³)—OR³, C(R³)—OR³, C—OR³, C(R³)—SR³, C—SR³, C—N₃, N—R³, N—OR³, N—N(R³)—R³, N—R⁴, or C=U with the proviso that:
(i) not more than two of W¹, W², W³, and W⁴ are a single bond; or
(ii) not more than two of W¹, W², W³, and W⁴ are O, S, N—R³, N—R⁴, C—N(R³)—R³, C—N(R³)—N(R³)—R³, C—N(R⁴)—N(R³)—R³, C(R³)—N(R³)—OR³, C—N(R³)—OR³, C—SR³, N—R³, N—OR³ or N—N(R³)—R³; or
(iii) not more than two of W¹, W², W³, and W⁴ are C=U; or
(iv) if two of W¹, W², W³, and W⁴ is O or S then at least one carbon atom is present between them; or
(v) when one of W¹, W², W³, and W⁴ is CH, N, C—R³, C—R⁴, C—N(R³)—R³, C—N(R³)—N(R³)—R³, C—N(R⁴)—N(R³)—R³, C—N(R³)—OR³, C—OR³, C—SR³, N—R³, N—OR³, or N—N(R³)—R³, a double bond is formed within the condensed ring.
